(19) United States
(12) Patent Application Publication
(10) Pub. No.: US 2019/0345557 A1
(43) Pub. Date: Nov. 14, 2019

(54) ATRIAL FIBRILLATION POLYGENIC RISK SCORE

(71) Applicant: THE GENERAL HOSPITAL CORPORATION, BOSTON, MA (US)

(72) Inventors: AMIT V. KHERA, BOSTON, MA (US); DEREK KLARIN, BOSTON, MA (US); SEKAR KATHIRESAN, BOSTON, MA (US)

(21) Appl. No.: 16/510,766

(22) Filed: Jul. 12, 2019

Related U.S. Application Data
(63) Continuation-in-part of application No. 16/034,260, filed on Jul. 12, 2018.
(60) Provisional application No. 62/718,352, filed on Aug. 13, 2018.
(60) Provisional application No. 62/585,378, filed on Nov. 13, 2017.
(60) Provisional application No. 62/583,997, filed on Nov. 9, 2017.
(60) Provisional application No. 62/531,762, filed on Jul. 12, 2017.

(51) Int. Cl. C12Q 1/6883; C12Q 1/6827

(52) U.S. Cl. CPC C12Q 1/6883; C12Q 1/6827; G01N 33/6893; C12Q 2600/158; C12Q 2600/106; C12Q 2600/156

(57) ABSTRACT

The present disclosure relates to a method of determining a risk of developing atrial fibrillation in a subject, the method comprising identifying whether at least 95 single nucleotide polymorphisms (SNPs) from Table A is present in a biological sample from the subject, wherein the presence of a risk allele of a SNP from Table A indicates that the subject has an increased risk of atrial fibrillation, and wherein the presence of an alternative allele indicates that the subject has a decreased risk of atrial fibrillation.

### CROSS-REFERENCE TO RELATED APPLICATIONS

This application is a continuation-in-part of prior U.S. patent application Ser. No. 16/034,260, filed Jul. 12, 2018, which claims the benefit of U.S. Provisional Application No. 62/531,762, filed Jul. 12, 2017, U.S. Provisional Application No. 62/583,997, filed Nov. 9, 2017, and U.S. Provisional Application No. 62/585,378, filed Nov. 13, 2017. This application claims the benefit of U.S. Provisional Application No. 62/718,352, filed Aug. 13, 2018. The entire contents of the above-identified applications are hereby fully incorporated herein by reference.

### STATEMENT REGARDING FEDERALLY SPONSORED RESEARCH

This invention was made with government support under Grant Nos. HL127564 and HG008895 awarded by the National Institutes of Health. The government has certain rights in the invention.

### REFERENCE TO AN ELECTRONIC SEQUENCE LISTING

The contents of the electronic sequence listing (“BROD-3780US_ST25.txt”; Size is 4,382 bytes and it was created on Jul. 12, 2019) is herein incorporated by reference in its entirety.

### TECHNICAL FIELD

The subject matter disclosed herein is generally directed to identifying individuals with a genetic redisposition to atrial fibrillation. In particular, the disclosure relates to a method for determining a risk of developing atrial fibrillation in a subject, and in some instances, providing a treatment to those determined to have an increased genetic risk.

### BACKGROUND

An increased risk of myocardial infarction in those with a parental history was first documented in 1951 (see Gertler et al., J. Am. Med. Ass., 1951; 147(7):621-25), catalyzing efforts to identify the discrete DNA-based drivers of heritable risk. A molecular defect in the gene encoding the LDL receptor (LDLR) was identified as a driver of hypercholesterolemia and coronary risk in 1985. (See Lehrman et al., Science, 1985; 227(4683):140-46). Subsequent genome-wide association studies (GWAS) were performed based on arrays designed to capture variants common in the population. The first such analyses for coronary disease uncovered multiple risk variants in the chromosomal 9p21 locus in 2007. (See Samani et al., N. Eng. J. Med., 2007; 357:443-53; Helgadottir et al., Science, 2007; 316:1491-1493; McPherson et al., Science, 2007; 316:1488-1491). Since then, more than 60 common genetic variants have been identified in progressively larger GWAS studies. (See Myocardial Infarction Genetics Consortium, Kathiresan S, Voight B F, et al., Nat Genet., 2009; 41(3):334-41; CARDIoGRAMplusC4D Consortium, Deloukas P, Kanoni S, et al., Nat Genet., 2013; 45:25-33; Nikpay et al., Nat Genet. 2015; 47(10):1121-30; Myocardial Infarction Genetics and CARDIoGRAM Exome Consortia Investigators, Stitziel N O, Stirrups K E, et al., N Engl J Med., 2016; 374(12):1134-44; Webb et al., J Am Coll Cardiol, 2017; 69(7):823-836). Furthermore, candidate gene analysis and whole exome sequencing, which captures variation in the 1% of the genome that encodes proteins, have associated a cumulative burden of rare, damaging variants in at least 9 genes with coronary risk. (See Do et al., Nature, 2015; 518(7537):102-6; Cohen et al., N Engl J Med., 2006; 354(12):1264-72; Myocardial Infarction Genetics Consortium Investigators, Stitziel N O, Won H H, et al., N Engl J Med., 2014; 371(22):2072-82; Nioi et al., N Engl J Med., 2016; 374(22):2131-41; Jorgensen et al., N Engl J Med., 2014 Jul. 3; 371(1):32-41; Crosby et al., Loss-of-function mutations in APOC3, triglycerides, and coronary disease, N Engl J Med., 2014; 371:22-31; Dewey et al., N Engl J Med., 2016; 374(12):1123-33; Khera et al., JAMA, 2017; 317(9):937-946).

Atrial fibrillation is an underdiagnosed and often asymptomatic disorder in which an irregular heart rhythm predisposes to blood clots and is a leading cause of ischemic stroke. The polygenic predictor identified 6.1% of the population at ≥3-fold risk and the top 1% had 4.63-fold risk. Screening for atrial fibrillation has become increasingly feasible owing to the development of ‘wearable’ device technology; these efforts to increase detection may have maximal utility in those with high GPS_{AF}.

Citation or identification of any document in this application is not an admission that such document is available as prior art to the present invention.

### SUMMARY

In one aspect, the disclosure relates to a method of determining a risk of developing atrial fibrillation in a subject, the method comprising: identifying whether at least 95 single nucleotide polymorphisms (SNPs) from Table A is present in a biological sample from the subject; wherein the presence of a risk allele of a SNP from Table A indicates that the subject has an increased risk of atrial fibrillation, and wherein the presence of an alternative allele indicates that the subject has a decreased risk of atrial fibrillation. In another aspect, the invention relates to a method of determining the risk of developing atrial fibrillation comprising odds ratios that are improved over method in the prior art. In some embodiments, the method further comprises calculating a polygenic risk score (PRS). In some embodiments, the PRS is calculated by summing the weighted risk score associated with each SNP identified. In some embodiments, identifying comprises measuring the presence of the at least 95 SNPs in the biological sample.

The invention relates to a method of determining a polygenic risk score for (PRS) developing atrial fibrillation in a subject, the method comprising selecting at least 50 single nucleotide polymorphisms (SNPs) from Table A; identifying whether the at least 50 SNPs are present in a biological sample from the subject; and calculating the polygenic risk score (PRS) based on the presence of the SNPs.

In some embodiments, the method further comprises assigning the subject to a risk group based on the PRS. In some embodiments, the method further comprises an initial step of obtaining a biological sample from the subject. In some embodiments, at least 100 SNPs are identified. In some embodiments, at least 200 SNPs, or at least 500 SNPs, or at least 1000 SNPs, or at least 2000 SNPs, or at least 5000 SNPs, or at least 10,000 SNPs, or at least 20,000 SNPs, or at least 50,000 SNPs, or at least 75,000 SNPs, or at least 100,000 SNPs, or at least 500,000 SNPs, or at least 1,000,000 SNPs, or at least 2,000,000 SNPs, or at least 3,000,000 SNPs, or at least 4,000,000 SNPs, or at least 5,000,000 SNPs, or at least 6,000,000 SNPs are identified. In some embodiments, the identified SNPs comprise the highest risk SNPs. In some embodiments, the identified SNPs comprise one or more of rs10841443, rs2244608, rs7500448, rs2972146, rs2972146, and rs11057401. In some embodiments, the method further comprises initiating a treatment to the subject. In some embodiments, the treatment is determined or adjusted according to the risk of atrial fibrillation. In some embodiments, the treatment comprises statins, ezetimibe, beta-blocking agents, angiotensin-converting-enzyme inhibitors, aspirin, anticoagulants, antiplatelet agents, angiotensin II receptor blockers, angiotensin receptor neprilysin inhibitors, calcium channel blockers, cholesterol-lowering medications, vasodilators, antidiuretics, renin-angiotensin system agents, lipid-modifying medicines, anti-inflammatory agents, nitrates, antiarrhythmic medicines, steroidal or non-steroidal anti-inflammatory drugs, DNA methyltransferase inhibitors and/or histone deacetylase inhibitors. In some embodiments, identifying whether the SNP is present comprises sequencing at least part of a genome of one or more cells from the subject. In some embodiments, the DNA methyltransferase inhibitors comprise 5-aza-2′-deoxycytidine or 5-azacytidine. In some embodiments, the histone deacetylase inhibitors comprise varinostat, romidepsin, panobinostat, belinostat or entinostat. In some embodiments, the lipid-modifying medicines comprise an antagonist of PCSK9, an antisense oligonucleotide targeting apolipoprotein C-III, and an antisense oligonucleotide to lower lipoprotein(a). In some embodiments, the statins comprise atorvastatin, fluvastatin, lovastatin, pravastatin, rosuvastatin, and simvastatin. In some embodiments, the subject is a human. In some embodiments, sequencing comprises whole genome sequencing.

The invention relates to a method of identifying a risk of developing atrial fibrillation, in a subject and providing a treatment to the subject, the method comprising obtaining a biological sample from the subject; identifying whether at least one single nucleotide polymorphism (SNP) from Table A is present in the biological sample; wherein the presence of a risk allele of a SNP from Table A indicates that the subject has an increased risk of atrial fibrillation; and initiating a treatment to the subject, wherein the treatment comprises statins, ezetimibe, beta-blocking agents, angiotensin-converting-enzyme inhibitors, aspirin, anticoagulants, antiplatelet agents, angiotensin II receptor blockers, angiotensin receptor neprilysin inhibitors, calcium channel blockers, cholesterol-lowering medications, vasodilators, antidiuretics, renin-angiotensin system agents, lipid-modifying medicines, anti-inflammatory agents, nitrates, antiarrhythmic medicines, steroidal or non-steroidal anti-inflammatory drugs, DNA methyltransferase inhibitors and/or histone deacetylase inhibitors.

The invention relates to a method of reducing a risk of atrial fibrillation, in a subject comprising administering to the subject a treatment which comprises one or more statins, beta-blocking agents, angiotensin-converting-enzyme inhibitors, aspirin, anticoagulants, antiplatelet agents, angiotensin II receptor blockers, angiotensin receptor neprilysin inhibitors, calcium channel blockers, cholesterol-lowering medications, vasodilators, antidiuretics, renin-angiotensin system agents, lipid-modifying medicines, anti-inflammatory agents, nitrates, antiarrhythmic medicines, steroidal or non-steroidal anti-inflammatory drugs, DNA methyltransferase inhibitors and/or histone deacetylase inhibitors, wherein the subject has a polygenic risk score that corresponds to a high risk group, and wherein the polygenic risk score is calculated by a method comprising selecting at least 95 single nucleotide polymorphisms (SNPs) from Table A; identifying whether the at least 95 SNPs are present in a biological sample from the subject; and calculating the polygenic risk score (PRS) based on the presence of the SNPs.

In another aspect, the invention relates to a method of detecting single nucleotide polymorphisms in a subject, said method comprising: detecting whether at least 95 single nucleotide polymorphisms (SNPS) from Table A are present in a biological sample from a subject by contacting the biological sample with a set of probes to each SNP and detecting binding of the probes, by amplifying genome regions comprising the SNPs using a set of amplification primers, or by sequencing genomic regions comprising or enriched for the SNPs. In some embodiments, at least 100 SNPs are detected. In some embodiments, at least 200 SNPs, or at least 500 SNPs, or at least 1000 SNPs, or at least 2000 SNPs, or at least 5000 SNPs, or at least 10,000 SNPs, or at least 20,000 SNPs, or at least 50,000 SNPs, or at least 75,000 SNPs, or at least 100,000 SNPs, or at least 500,000 SNPs, or at least 1,000,000 SNPs, or at least 2,000,000 SNPs, or at least 3,000,000 SNPs, or at least 4,000,000 SNPs, or at least 5,000,000 SNPs, or at least 6,000,000 SNPs are detected.

The invention relates to a method of determining a risk of developing atrial fibrillation in a subject, the method comprising identifying whether at least 95 single nucleotide polymorphisms (SNPs) from Table A is present in a biological sample from the subject and calculating a polygenic risk score (PRS); wherein the presence of a risk allele of a SNP from Table A indicates that the subject has an increased risk of atrial fibrillation, and wherein the presence of an alternative allele indicates that the subject has a decreased risk of atrial fibrillation.

The invention relates to a method of determining a risk of developing atrial fibrillation in a subject, the method comprising obtaining a biological sample from the subject; identifying whether at least 95 single nucleotide polymorphisms (SNPs) from Table A is present in the biological sample from the subject and, optionally, calculating a polygenic risk score (PRS); wherein the presence of a risk allele of a SNP from Table A indicates that the subject has an increased risk of atrial fibrillation, and wherein the presence of an alternative allele indicates that the subject has a decreased risk of atrial fibrillation.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIGS. 1A-1B. FIG. 1A: Stage 1 consisted of a genome-wide association study for the coronary artery disease phenotype performed in UK Biobank; variants below a threshold P value <0.05 moving forward to meta-analysis with CARDIoGRAM Exome (Stage 2) or CARDIoGRAMplusC4D summary statistics (Stage 3). Abbreviations: 1000G, 1000 Genomes; CARDIoGRAMplusC4D, Coronary ARtery Disease Genome-wide Replication and Meta-analysis; MIGen, Myocardial Infarction Genetics. FIG. 1B: An expanded genome-wide polygenic score can identify individuals with 2.5-fold increased risk.

FIG. 2. Phenome-wide association results for 15 novel loci. For the 15 novel CAD risk variants identified in our study, Z-scores (aligned to the CAD risk allele) were obtained from the Genomics plc Platform and UK Biobank. A positive Z-score indicates a positive association between the CAD risk allele and the disease/trait, while a negative Z-score indicates an inverse association. Boxes are outlined in green if the variant is significantly (P<0.00013) associated with the given trait. Abbreviations: Adj, Adjusted; BMI, Body Mass Index; BP, Blood Pressure; crea, Creatinine; cys, cystatin-c; COPD, chronic obstructive pulmonary disease; eGFR, estimated Glomerular Filtration Rate; HDL, High Density Lipoprotein; LDL, Low Density Lipoprotein.

FIG. 3. Biological pathways underlying genetic loci associated with coronary artery disease. CAD GWAS loci identified to date are depicted along with the plausible relationship to the underling biological pathway. The 15 new loci described in this paper are shown in bold. Loci names are based on the nearest genes. Adapted from Ref (Khera, A. V. & Kathiresan, Nat Rev Genet 18, 331-344 (2017)).

FIGS. 4A-4C. Functional assessment of ARHGEF26 p.Val29Leu in vitro. FIG. 4A: ARHGEF26-29Leu increases leukocyte transendothelial migration. HAEC were transfected with non-targeting siRNA and empty vector (control), siRNA against ARHGEF26 3′-UTR and empty vector, siRNA and ARHGEF26-WT, or siRNA and ARHGEF26-29Leu. Transfected HAEC were plated on transwell inserts and treated with 10 ng/mL TNF-α. Differentiated HL60 cells were loaded on the upper chambers of transwells and allowed to transmigrate across HAEC towards vehicle (blue) or 50 ng/mL SDF-1 (red). The migrated cells were quantified as percentage of input cells per well (n=5 or 6; mean+s.d.; F=11.89, DF=3 by two-way ANOVA within vehicle and SDF-1 subgroups with Fisher's LSD test; variance among vehicle subgroups non-significant; NS, not significant; representative of 3 independent experiments). FIG. 4B: ARHGEF26-29Leu increases leukocyte adhesion on endothelial cells. HAEC were transfected as 2a) and cultured on 96-well plates until confluent and treated with 10 ng/mL TNF-α. Calcein-AM-labeled THP-1 cells were incubated with HAEC and washed to remove non-adherent cells. The adherent cells were lysed, quantified by Calcein-AM fluorescence and compared to siRNA+WT (n=25, 17, 20, and 17; mean+s.d.; F=14.53, DF=3 by one-way ANOVA; NS, not significant; * P<0.0001 compared to siRNA+WT; representative of 3 independent experiments).

FIG. 4C: ARHGEF26-29Leu increases vascular smooth muscle cell proliferation. HCASMC were transfected as 2a) and made quiescent by serum starvation for 48 h, followed by 72-h proliferation in normal serum medium. Cell proliferation was quantified by a luminescent assay and compared to siRNA+WT (n=20; mean+s.d.; F=197.5, DF=3 by one-way ANOVA; NS, not significant; * P<0.0001 compared to siRNA+WT; representative of 3 independent experiments).

FIG. 5 depicts quantile-quantile plot for the Stage 1 CAD GWAS. The expected association P values versus the observed distribution of P values for CAD association is displayed. Significant systemic inflation is not observed (λ_{GC}=1.05).

FIG. 6 depicts Manhattan plot for the Stage 1 CAD GWAS. Plot of −log₁₀(P) for association of imputed variants by chromosomal position for all autosomal polymorphisms analyzed in the UK Biobank, Stage 1 CAD GWAS. The genes nearest to the top associated variants are displayed. Abbreviations: CAD, coronary artery disease; GWAS, genome-wide association study.

FIG. 7 depicts risk allele effect estimates in the literature and in UK biobank for a set of previously reported CAD variants. Plot of the effect estimates for 56 CAD associated DNA sequence variants as reported in the 1000G imputed CARDIoGRAMplusC4D analysis¹ and in our UK Biobank GWAS analysis. 3=0.92, 95% CI: 0.77-1.06; P=1.8×10¹⁷.

FIGS. 8A-8D depicts Stage 2 regional association plots for novel CAD loci LOC646736 (FIG. 8A), CCDC92 (FIG. 8B), ARHGEF26 (FIG. 8C) and LOX (FIG. 8D). These regional association plots demonstrate the strength of association, by −log₁₀(p-value), for four of the novel CAD loci in Stage 2, within a window of +/−400 kilobases.

FIGS. 9A-9F depicts regional association plots for novel CAD loci FN1 (FIG. 9A), UMPS-ITGB5 (FIG. 9B), FGD5 (FIG. 9C), RHOA (FIG. 9D), FGF5 (FIG. 9E), and MAD2L1 (FIG. 9F). These regional association plots demonstrate the strength of association, by −log₁₀(p-value), for six novel CAD loci in Stage 3, within a window of +/−400 kilobases.

FIGS. 10A-10E depicts stage 3 regional association plots for novel CAD loci RP11-664H17.1 (FIG. 10A), HNF1A (FIG. 10B), CFDP1 (FIG. 10C), CDH13 (FIG. 10D), and TGFB1 (FIG. 10E). These regional association plots demonstrate the strength of association, by −log₁₀(p-value), for five novel CAD loci in Stage 3, within a window of 400 kilobases.

FIGS. 11A-11B illustrates the analyses of gene expression associated with the rs12493885 alleles. FIG. 11A: eQTL analysis. In 133 coronary artery samples obtained by GTEx, eQTL analysis does not demonstrate evidence of altered expression associated with the ARHGEF26 p.Val29Leu (rs12493885) variant. 3=0.22, P=0.16. No other variants in the region demonstrate significant eQTL effects at an FDR <0.05 threshold in coronary artery. FIG. 11A: Allele specific expression analysis. In 20 coronary artery samples obtained from the GTEx Consortium heterozygous for the ARHGEF26 p.Val29Leu (rs12493885) variant, no individual demonstrated significant evidence of allele imbalance in coronary artery at an FDR <0.05 threshold (n.s.: two-sided binomial test non-significant). REF refers to the reference (G) and ALT to the alternative (C) allele.

FIG. 12 illustrates ARHGEF26 promoter activity luciferase assay. The −2516 to +2 region 5′ of ARHGEF26 gene were cloned for haplotypes of rs12493885 G (reference) and C (alternative) alleles, respectively. The reference and alternative haplotypes were coupled with a firefly luciferase reporter and co-transfected with a Renilla luciferase co-reporter in HEK293 cells, HAEC, and HUVEC. Promoter-less firefly luciferase reporter was included as negative control. Firefly luciferase activity relative to Renilla luciferase was measured 48 hours post-transfection, and expressed as fold changes over promoterless vectors (HEK293 n=4, HAEC n=6, and HUVEC n=6; mean+s.d.; separate one-way ANOVA with Tukey's multiple comparisons tests and multiplicity adjusted P values for each cell type; F=23.88, DF=2 for HEK293; F=0.8038, DF=2 in HAEC; F=0.02397, DF=2 in HUVEC).

FIG. 13 shows western blots of transfected vascular cells. HAEC or HCASMC were transfected with non-targeting siRNA plus empty vector (Control), siRNA against ARHGEF26 3′ UTR and empty vector (siRNA+empty vector), siRNA and a wild-type FLAG-ARHGEF26 vector (siRNA+WT), or siRNA and a mutant vector (siRNA+29Leu). Transfected HAEC or HCASMC was harvested 72-hour post-transfection. Normalized cell lysates (20 μg/lane) were resolved by SDS-PAGE and probed for ARHGEF26, FLAG, and actin by respective antibodies and imaged by enhanced chemiluminescence.

FIG. 14 shows the effects of p. Val29Leu mutant on ARHGEF26 protein quality. Evaluation of ARHGEF26 wild-type and 29Leu nucleotide exchange activity. Full-length, N-terminal His-SUMO-tagged wild-type and 29Leu ARHGEF26 and full-length RhoG were expressed in E. coli. Nucleotide exchange assay was prepared with equal amount of recombinant ARHGEF26-WT (blue) and ARHGEF26-29Leu (red) in reaction buffer containing MANT-GTP. Just prior to reading, recombinant RhoG protein, pre-loaded with GDP, was added to the reaction buffer at a final concentration of 0.4 μM. MANT-GTP fluorescence was monitored for 60 minutes on a SpectraMax M2 at 37° C. using an excitation wavelength of 280 nm and an emissions wavelength of 440 nm with a 435 nm cutoff. No significant difference in nucleotide exchange activity was observed between ARHGEF26-WT (blue) and ARHGEF26-29Leu (red) in the presence of RhoG.

FIG. 15 depicts evaluation of ARHGEF26 protein stability in cells. Wild-type (WT) or 29Leu FLAG-ARHGEF26 were overexpressed in HEK293 cells for 48 hours followed by treatment of 50 μg/mL and 100 μg/mL cycloheximide. Cells were harvested at indicated time points post treatment, and normalized lysate (20 μg/lane) were probed for FLAG by Western blot. For each cycloheximide dose, 2 blot sections (WT and 29Leu) from the same membrane simultaneously imaged are shown in juxtaposition for contrast.

FIG. 16 depicts the principal components of ancestry according to myocardial infarction status and race. Principal components of ancestry were calculated based on approximately 16,000 ancestry-informative markers. Display of the first two principal components by myocardial infarction case status and race demonstrates confirms similar ancestral background across studies.

FIGS. 17A-17C shows a spectrum of consequences and allelic frequency of identified genetic variants. Observed variants were annotated using the Ensembl Variant Effect Predictor⁴⁰ ‘Consequence’ field. FIG. 17A: The percent of all observed variants that fall into each category of annotation is displayed. FIG. 17B: The percent of observed protein-coding variant (1.2% of overall sample) that fall into each annotation category is displayed. FIG. 17C: The percent of observed variants that fall into various categories of allele frequency is displayed, including 54.9% that were observed in only a single individual (Singleton), 22.7% with 2-7 observed alleles, 12.3% with allele frequency up to 0.5%, 5.4% with allele frequency >0.5% but less than 5%, and 4.7% with frequency >5%.

FIG. 18 illustrates the monogenic risk pathways and risk of early-onset myocardial infarction. Ascertainment of rare, damaging mutations in genes related to familial hypercholesterolemia (LDLR, APOB) or impaired clearance of triglycerides (LPL, APOA5) was performed. Individuals with at least two variants at the LPA genetic locus previously shown to relate to increased lipoprotein(a) and risk of coronary artery disease (rs10455872 and rs3798220) were also included. (See Clarke et al., N Engl J Med., 2009; 361(26):2518-28).

FIG. 19 shows a comparison of new polygenic risk score to previously published scores in the whole-genome sequencing dataset. Individuals were stratified into high (top quintile of polygenic score), intermediate (quintiles 2-4), and low (lowest quintile of polygenic score). Relationship of these strata to odds of myocardial infarction was compared among for two previously published scores and the new expanded polygenic score. The expanded score had improved predictive ability as compared to either previous score (P<0.0001 for each by likelihood ratio test).

FIG. 20 shows a comparison of polygenic risk score association with myocardial infarction within racial subgroups. The association of polygenic risk score categories was assessed within each racial subgroup using logistic regression adjusted for principal components of ancestry. Stronger associations were noted in White as compared to non-White individuals (p-interaction=0.001).

FIGS. 21A-21D illustrates the sequencing quality metrics according to case-control status. FIG. 21A. As expected based on target mean coverage of >30× for the MESA cohort and >20× for the VIRGO and TAICHI studies, mean depth was slightly lower in myocardial infarction cases as compared to controls (32.8 versus 29.5 respectively). Despite this, sequencing quality metrics were similar across case and control individuals in race-stratified analyses: FIG. 21B. Total number of single nucleotide polymorphisms (SNPs); FIG. 21C. Transition to Tranversion Ratios; D. Ratio of heterozygote/homozygote genotype calls.

FIGS. 22A-22D shows the common and rare variant genetic association analyses. Quantile-quantile plots demonstrating observed versus expected p-value distributions are provided for relationship with early-onset myocardial infarction in analyses adjusted for principal components of ancestry, including FIG. 22A. common (allele frequency >0.01) single nucleotide polymorphisms; FIG. 22B. common insertion-deletion variants; FIG. 22C. rare coding variant (allele frequency <0.01) gene burden tests; FIG. 22D. rare noncoding variants in aortic tissue regulatory region burden tests.

FIG. 23 shows a heatmap of area under the curve for polygenic risk score association with coronary artery disease in the UK Biobank. Model discrimination for coronary artery disease (CAD) as assessed by area under the curve (AUC) using 24 potential polygenic risk scores (PRS). Scores were derived across a range of p-value and r² thresholds using the --clump procedure in PLINK 1.90b based on 1000 Genomes imputed GWAS statistics and LD from 1000 Genomes Phase 1 version 3. Each score was assessed using logistic regression on 4831 CAD cases and 115,455 controls of European Ancestry in the UK Biobank, adjusting for the first four PCs of ancestry. Shading represents the magnitude of the AUC with darker shades representing better model discrimination.

FIG. 24. Study design. Score derivation was performed using summary association statistics from the previously published CARDIOGRAMplusC4D genome-wide association study.¹⁶ The correlation of these variants were assessed in 503 European individuals from 1000 Genomes phase 3 version 5.¹⁷ The testing dataset to choose the optimal score included 120,286 individuals of European ancestry from the UK Biobank Phase I genotype release, of whom 4,831 had CAD.¹⁸ Validation datasets included a multiethnic case-control cohort of early-onset (age <60 years) CAD and disease free controls. Cases were derived from the VIRGO (Variation in Recovery: Role of Gender on Outcomes of Young AMI Patients) and TAICHI consortium and controls from the MESA (Multi-Ethnic Study of Atherosclerosis) cohort and TAICHI consortium. Additional validation of prevalent CAD was performed in individuals of European ancestry from the UK Biobank Phase II genotype release—inclusive of 8,676 individuals with CAD and 280,304 controls. The association of the polygenic score with incident CAD events was assessed in the 280,304 individuals of the UK Biobank Phase II genotype release free of CAD at baseline and 7,318 individuals of European ancestry from the ARIC (Atherosclerosis Risk in Communities) prospective cohort.

FIGS. 25A-25B. Polygenic score distribution and association with CAD in the testing dataset. FIG. 25A. The distribution of the 6,630,150 variant polygenic score in the testing dataset derived from the UK Biobank Phase I genotype release. The x-axis represents the polygenic score, with values scaled to a mean of 0 and standard deviation of 1 to facilitate interpretation. The y-axis corresponds to the frequency among 120,286 individuals of the testing dataset. FIG. 25B. The population was divided into low (bottom quintile), intermediate (quintile 2-4), and high (top quintile) of polygenic risk. The association of the polygenic score with CAD in the testing dataset was assessed using logistic regression adjusting for the first four principal components of ancestry. This score had improved discrimination as compared to a previously published score restricted to 50 variants that had achieved genome-wide significance (p<0.001).

FIG. 26. Association of the polygenic score with early-onset CAD in a multiethnic population. The relationship of low (bottom quintile), intermediate (quintile 2-4), and high (top quintile) of polygenic risk with early-onset CAD was determined in a case-control cohort derived from the VIRGO-MESA-TAICHI) studies, with quintiles determined in a race-specific fashion. The odds of early-onset CAD in those with intermediate or high polygenic risk was compared to a reference group with low polygenic risk using logistic regression adjusted for the first four principal components of ancestry. The polygenic score categories were more strongly associated with early-onset CAD in white as compared to non-white participants (p-value for heterogeneity by race <0.001).

FIGS. 27A-27C. Association of the polygenic score with prevalent and incident CAD in the UK Biobank. Within the UK Biobank Phase II genotype release validation cohort, individuals were stratified into low (bottom quintile of polygenic score), intermediate (quintiles 2-4), and high (top quintile of polygenic score) polygenic risk. FIG. 27A. The relationship of these risk categories to prevalent disease among 288,980 individuals (8,676 individuals with CAD and 280,304 controls) was tested using logistic regression adjusted for the first four principal components of ancestry and a dummy variable representing genotyping array. FIG. 27B. Incident CAD events among 280,304 individuals free of CAD at time of recruitment. Cumulative hazard survival curves displayed according to polygenic risk category. FIG. 27C. Multivariable model for the association of polygenic score categories with incident CAD events including adjustment for traditional cardiovascular risk factors. Hazard ratios represent effect estimates from a multivariable model including all displayed variables, as well as the first four principal components of ancestry and a dummy variable representing genotyping array.

FIGS. 28A-28C. Association of the polygenic score with incident CAD in the Atherosclerosis Risk in Communities Study. Within the Atherosclerosis Risk in Communities validation cohort of 7,318 white individuals, participants were stratified into low (bottom quintile of polygenic score), intermediate (quintiles 2-4), and high (top quintile of polygenic score) polygenic risk. FIG. 28A. Cumulative hazard survival curves displayed according to polygenic risk category. FIG. 28B. The relationship of polygenic scores with 10-year risk of coronary events according to predicted risk as assessed by the ACC/AHA Pooled Cohorts Equation. Adjusted 10-year risk was calculated using Cox regression, standardized to mean of covariates age, sex, and the first four principal components of ancestry. FIG. 28C. Multivariable model for the association of polygenic score categories with incident CAD events including adjustment for traditional cardiovascular risk factors. Hazard ratios represent effect estimates from a multivariable model including all displayed variables, as well as the first four principal components of ancestry.

FIG. 29. Relationship of the Polygenic Score to the ACC/AHA Pooled Cohorts Equation Ten-Year Risk in the Atherosclerosis Risk in Communities Study. The polygenic score was standardized (set to mean of 0 and standard deviation of 1) to facilitate interpretation. Minimal correlation was noted between this score and individuals 10-year risk of atherosclerotic cardiovascular disease as assessed by the ACC/AHA Pooled Cohorts Equations (Spearman r=0.03).

FIGS. 30A-30D. Sequencing Quality Metrics According to Case-Control Status in the VIRGO-MESA-TAICHI Validation Cohort. FIG. 30A. Based on target mean coverage of >30× for the MESA cohort and >20× for the VIRGO and TAICHI studies, mean depth was slightly lower in myocardial infarction cases as compared to controls (32.8 versus 29.5 respectively). Despite this, sequencing quality metrics were similar across case and control individuals in race-stratified analyses: FIG. 30B. Total number of single nucleotide polymorphisms (SNPs); FIG. 30C. Transition to Tranversion Ratios; FIG. 30D. Ratio of heterozygote/homozygote genotype calls.

FIGS. 31A-31B. A new genome wide polygenic score (PS_{GW}) identifies individuals with significantly increased risk of coronary disease. A near normal distribution of the PS_{GW} was noted in the UK Biobank validation cohort (FIG. 31A). The x-axis represents PS_{GW}, with values scaled to a mean of 0 and standard deviation of 1 to facilitate interpretation. Individuals were binned into 40 groups based on PS_{GW}, with each grouping representing 2.5% of the population (˜7225 individuals). The high polygenic risk group displayed in red (top 2.5% of the distribution) had a significantly higher prevalence of coronary disease (FIG. 31B).

FIG. 32. 157,897 female participants of the UK Biobank validation dataset were binned into 40 groups based on the PS_{GW} for breast cancer with each grouping representing 2.5% of the population (˜3947 individuals). The high polygenic risk group displayed in red (top 2.5% of the distribution) had a significantly higher prevalence of breast cancer (p<0.0001).

FIG. 33. 288,180 individuals of the UK Biobank validation dataset were binned into 40 groups based on the PS_{GW} for body-mass index, with each grouping representing 2.5% of the population (˜7200 individuals). The high polygenic risk group displayed in red (top 2.5% of the distribution) had a significantly higher prevalence of severe obesity (p<0.0001).

FIGS. 34A-34B. FIG. 34A. Polygenic score distribution of 6.6 million common variants and corresponding odds ratio to the high polygenic score definition. FIG. 34B. Odds ratio for top 20% of the score distribution according to race.

FIGS. 35A-35C. FIG. 35A. Polygenic score distribution of 6.6 million common variants for high polygenic score definition of top 20%, top 10%, top 2.5%, top 1% and top 0.25%. FIG. 35B. Prevalence of coronary artery disease (CAD) across polygenic score percentiles. FIG. 35C. Incident CAD events across polygenic score percentiles.

FIG. 36. Standardized coronary events rates, according to genetic and lifestyle risk in the prospective cohorts. Within each cohort, the percentages in black font refer to the number of individuals in each category of lifestyle risk. For each lifestyle risk category, the percentage of individuals in each genetic risk category is displayed in white font. P-values for association between genetic and lifestyle risk categories 0.41, 0.95, 0.832, and 0.30 in ARIC WGHS, MDCS, and BioImage cohorts respectively.

FIG. 37. Risk of coronary events, according to genetic and lifestyle risk in the prospective cohorts. Average (Range) genetic risk scores were 3.53 (2.15-4.87) in ARIC, 3.66 (2.33-5.41) in WGHS, 3.82 (2.20-5.71) in MDCS and 3.54 (2.07-4.90) in the BioImage Study. Variation in scores across cohorts was related to slight differences in number of available component SNPs as noted in Table 12.

FIGS. 38A-38C. Standardized Coronary Events Rates, According to Genetic and Lifestyle Risk in the Prospective Cohorts. Shown are the standardized rates of coronary events, according to the genetic risk and lifestyle risk of participants in (FIG. 38A) the Atherosclerosis Risk in Communities (ARIC) cohort, (FIG. 38B) the Women's Genome Health Study (WGHS) cohort, and (FIG. 38C) the Malmö Diet and Cancer Study (MDCS) cohort. The 95% confidence intervals for the hazard ratios are provided in parentheses. Cox regression models were adjusted for age, sex (in ARIC and MDCS), randomization to receive vitamin E or aspirin (in WGHS), education level, and principal components of ancestry (in ARIC and WGHS). Standardization was performed to cohort-specific population averages for each covariate.

FIG. 39. Unadjusted cumulative hazard plots by genetic and lifestyle risk category. Unadjusted incidence rates per 1000 person-years of follow-up are displayed for each category of genetic and lifestyle risk.

FIG. 40. Risk of Coronary Events, According to Genetic and Lifestyle Risk in the Prospective Cohorts. Shown are adjusted hazard ratios for coronary events in each of the three prospective cohorts, according to genetic risk and lifestyle risk. In these comparisons, participants at low genetic risk with a favorable lifestyle served as the reference group. There was no evidence of a significant interaction between genetic and lifestyle risk factors (P=0.38 for interaction in the Atherosclerosis Risk in Communities (ARIC) cohort, P=0.31 in the Women's Genome Health Study (WGHS) cohort, and P=0.24 in the Malmö Diet and Cancer Study (MDCS) cohort). Unadjusted incidence rates are reported per 1000 person-years of follow-up. A random-effects meta-analysis was used to combine cohort-specific results.

FIGS. 41A-41C. 10-Year Coronary Event Rates, According to Lifestyle and Genetic Risk in the Prospective Cohorts. Shown are standardized 10-year cumulative incidence rates for coronary events in the three prospective cohorts ((FIG. 41A) the Atherosclerosis Risk in Communities (ARIC) cohort, (FIG. 41B) the Women's Genome Health Study (WGHS) cohort, and (FIG. 41C) the Malmö Diet and Cancer Study (MDCS) cohort), according to lifestyle and genetic risk. Standardization was performed to cohort-specific population averages for each covariate. The I bars represent 95% confidence intervals.

FIG. 42. Sensitivity analysis: risk of myocardial infarction or death from coronary causes according to genetic and lifestyle category in prospective cohorts. Cox regression models were adjusted for age, gender (in ARIC and MDCS), randomization to Vitamin E or aspirin (in WGHS), education level, and principal components of ancestry (in ARIC and WGHS).

FIG. 43. Sensitivity analysis: risk of coronary events according to genetic and lifestyle category adjusted for traditional risk factors. Cox regression models were adjusted for age, gender (in ARIC and MDCS), randomization to Vitamin E or aspirin (in WGHS), education level, principal components of ancestry (in ARIC and WGHS), presence of diabetes mellitus, hypertension, family history of coronary artery disease, LDL cholesterol levels (apoliproprotein in B in MDCS), and HDL cholesterol levels (apoliproprotein A-I in MDCS).

FIG. 44. Risk of coronary events according to genetic and lifestyle category among black participants. Cox regression model was adjusted for age, gender, education level, and principal components of ancestry. 2,269 black participants of the ARIC study had genotype and covariate data available for analysis. 350 incident coronary events were observed during follow-up. Those at high genetic risk were at increased risk of coronary events (HR 1.65; 95% Cl 1.16-1.34; p=0.006) compared to those at low genetic risk. Furthermore, an unfavorable lifestyle was associated with a 70% increased coronary risk (HR 1.70; 95% Cl 1.20-2.39; p=0.003). As with white participants, risk of coronary events tended to decrease with adherence to a more favorable lifestyle within categories of low and intermediate genetic risk. This pattern was not apparent among those with a high genetic risk, potentially related to decreased power due to a small number of incident events.

FIG. 45. Coronary-Artery Calcification Score in the BioImage Study, According to Lifestyle and Genetic Risk. Among the participants in the BioImage Study, a standardized score for coronary-artery calcification was determined by means of linear regression after adjustment for age, sex, education level, and principal components of ancestry. Standardization was performed on the basis of study averages for each covariate. Average standardized coronary-artery calcification scores are expressed in Agatston units, with higher scores indicating an increased burden of coronary atherosclerosis. The I bars represent 95% confidence intervals.

FIG. 46 is a flow chart showing study design and workflow. A genome-wide polygenic score (GPS) for each disease was derived by combining summary association statistics from a recent large GWAS and a linkage disequilibrium reference panel of 503 Europeans. 31 candidate GPS were derived using two strategies: 1. ‘pruning and thresholding’—aggregation of independent polymorphisms that exceed a specified level of significance in the discovery GWAS and 2. LDPred computational algorithm, a Bayesian approach to calculate a posterior mean effect for all variants based on a prior (effect size in the prior GWAS) and subsequent shrinkage based on linkage disequilibrium. The seven candidate LDPred scores vary with respect to the tuning parameter p, the proportion of variants assumed to be causal, as previously recommended. The optimal GPS for each disease was chosen based on area under the receiver-operator curve (AUC) in the UK Biobank Phase I validation dataset (N=120,280 Europeans) and subsequently calculated in an independent UK Biobank Phase II testing dataset (N=288,978 Europeans).

FIGS. 47A-47C depict risk for coronary artery disease according to genome-wide polygenic score. FIG. 47A is a diagram showing distribution of genome-wide polygenic score for CAD (GPS_{CAD}) in the UK biobank testing dataset (N=288,978). The x-axis represents GPS_{CAD}, with values scaled to a mean of 0 and standard deviation of 1 to facilitate interpretation. Shading reflects proportion of population with 3, 4, and 5-fold increased risk versus remainder of the population. Odds ratio assessed in a logistic regression model adjusted for age, sex, genotyping array, and the first four principal components of ancestry. FIG. 47B is a graph showing GPS_{CAD} percentile among CAD cases versus controls in the UK biobank validation cohort. Within each boxplot, the horizontal lines reflect the median, the top and bottom of the box reflects the interquartile range, and the whiskers reflect the maximum and minimum value within each grouping. FIG. 47C is a graph showing prevalence of CAD according to 100 groups of the validation cohort binned according to percentile of the GPS_{CAD}.

FIGS. 48A-48C depict risk gradient for coronary artery disease across the distribution of the genome-wide polygenic score and two previously published scores. Three polygenic scores for coronary artery disease were calculated within the UK Biobank testing dataset of 288,978 participants. FIG. 48A is a graph showing a previously published score comprised of 50 variants that had achieved genome-wide levels of statistical significance in previous studies (Tada H, et al. Eur Heart J. 37, 561-7, 2016). FIG. 48B is a graph showing a previously published score comprised of 49,310 variants derived from a Metabochip GWAS study (Abraham G., et al. Eur Heart J. 37, 3267-3278, 2016). FIG. 48C is a graph showing the newly derived genome-wide polygenic score comprised of 6,630,150 variants. For each score, the population was divided into 100 bins according to percentile of the score and prevalence of coronary artery disease within each bin plotted. The prevalence of coronary artery disease across score percentiles ranged from 1.4 to 5.9% for the 50-variant score, 1.0 to 7.2% for the 49,310 variant score, and 0.8 to 11.1% for the 6,630,150-variant genome-wide polygenic score.

FIGS. 49A-49D depict risk gradient for disease according to genome-wide polygenic score percentile. 100 groups of the validation cohort were derived according to percentile of the disease-specific GPS. FIG. 49A is a graph showing prevalence of disease displayed for risk of atrial fibrillation according to GPS percentile. FIG. 49B is a graph showing prevalence of disease displayed for risk of type 2 diabetes according to GPS percentile. FIG. 49C is a graph showing prevalence of disease displayed for risk of inflammatory bowel disease according to GPS percentile. FIG. 49D is a graph showing prevalence of disease displayed for risk of breast cancer according to GPS percentile

FIG. 50 is a graph depicting predicted versus observed prevalence of coronary artery disease according to genome-wide polygenic score percentile. For each individual within the UK Biobank testing dataset, the predicted probability of disease was calculated using a logistic regression model with only the genome-wide polygenic score (GPS) as a predictor. The predicted prevalence of disease within each percentile bin of the GPS distribution was calculated as the average predicted probability of all individuals within that bin. The shape of the predicted risk gradient was consistent with the empirically observed risk gradient, reflected by black and blue dots, respectively.

FIGS. 51A-51D depict predicted versus observed prevalence of four disease according to genome-wide polygenic score percentile. For each individual within the UK Biobank testing dataset, the predicted probability of disease was calculated using a logistic regression model with only the genome-wide polygenic score (GPS) as a predictor. The predicted prevalence of disease within each percentile bin of the GPS distribution was calculated as the average predicted probability of all individuals within that bin. The shape of the predicted risk gradient was consistent with the empirically observed risk gradient, reflected by black and blue dots, respectively, for each of four diseases. FIG. 51A is a graph showing the predicted risk gradient and the observed risk gradient for atrial fibrillation. FIG. 51B is a graph showing the predicted risk gradient and the observed risk gradient for type 2 diabetes. FIG. 51C is a graph showing the predicted risk gradient and the observed risk gradient for inflammatory bowel disease. FIG. 51D is a graph showing the predicted risk gradient and the observed risk gradient for breast cancer. Breast cancer analysis was restricted to female participants.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2d edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

The present disclosure relates to Applicant's findings that lead to the development of a genetic predictor that can identify a subset of the population at more than 4-fold higher risk for atrial fibrillation. This is among the strongest predictors ever developed such application. In certain embodiments, determination of the presence or absence of risk alleles is followed by calculating the polygenic risk score for the subject, wherein a high polygenic score indicates a higher risk for developing atrial fibrillation.

In one aspect, the present disclosure provides methods of determining a risk of developing atrial fibrillation in a subject. In general the method may comprise identifying whether a group of SNPs are present in a biological sample from the subject. In some embodiments, the group SNPs comprises at least 95 SNPs from Table A, which includes a list of variants and weighs comprising polygenic risk scores for atrial fibrillation, disclosed in Amit V. Khera, et al., Genome-wide polygenic scores for common diseases identify individuals with risk equivalent to monogenic mutations, Nature Genetics, 2018, 50:1219-1224 (doi.org/10.1038/s41588-018-0183-z) (“Khera”), which is incorporated herein by reference by its entirety. In regards to Table A, Applicant specifically references the data referred to on the seventh page of Khera under “Data Availability” as available at www.broadcvdi.org/informational/data (“Polygenic Risk Score Variant Weights”). Table A refers specifically to the Polygenic Risk Score Variant Weights table named “Atrial fibrillation” and having a size of 297.3 MB.

With the group of SNPs, a polygenic risk score (PRS) for developing atrial fibrillation may be calculated. In some embodiments, the method further comprising administering a treatment (e.g., a treatment of atrial fibrillation) to the subject. The treatment may be designed or planned based on the PSR.

### Methods of Diagnosis and Risk Determination

The present disclosure provides methods for diagnosing a disease or condition (e.g., atrial fibrillation or related diseases), and/or or determining the risk of developing the disease or condition. According to the invention, genomic sequences associated with disease risk are identified by single nucleotide polymorphisms (SNPs). The SNPs are linked to the genomic sequences of interest, i.e., close to or within the genomic sequences of interest, and may or may not be causative of the risk variation. That is, functional differences between alleles distinguished by the SNPs may result from sequence variation of an SNP or from one or more differences between alleles located near to the location of the SNP. In either case, the invention provides for gene editing in order to reduce disease risk. In general, a higher risk allele would be edited, for example, to a lower risk allele. Often such editing would involve individual base changes, but can also involve insertions and deletions. For example, trinucleotide repeat regions may be edited to change the number of trinucleotide repeats.

Risk assessments using large numbers of SNPs offers the advantage of increased predictive power. In certain embodiments, the invention includes in the risk assessment large numbers of alleles, for example, at least 500,000, at least 1,000,000, at least 2,000,000, at least 3,000,000, at least 4,000,000, at least 5,000,000, or at least 6,000,000 SNPs from Table A.

In some embodiments, the present disclosure provides to a method of determining a risk of developing atrial fibrillation, in a subject, the method comprising identifying whether at least 50, at least 95, at least 100, at least 200, at least 500, at least 1000, at least 2000, at least 5000, at least 10,000, at least 20,000, at least 50,000, at least 75,000, or at least 100,000 SNPs single nucleotide polymorphisms (SNPs) from Table A is present in a biological sample from the subject; wherein the presence of a risk allele of a SNP from Table A indicates that the subject has an increased risk of the disease, and wherein the presence of an alternative allele indicates that the subject has a decreased risk of atrial fibrillation.

In an embodiment, the invention provides a method of determining a risk of developing atrial fibrillation in a subject comprising identifying whether the SNPs from Table A is present in a biological sample from the subject and calculating a polygenic risk score (PRS) for the subject based on the identified SNPs. The number of identified SNPs can be at least 50, at least 95, at least 100, at least 200, at least 500, at least 1000, at least 2000, at least 5000, at least 10,000, at least 20,000, at least 50,000, at least 75,000, or at least 100,000.

In an embodiment, the invention provides a method of determining a risk of developing atrial fibrillation in a subject, the method comprising identifying whether at least 50, at least 95, at least 100, at least 200, at least 500, at least 1000, at least 2000, at least 5000, at least 10,000, at least 20,000, at least 50,000, at least 75,000, or at least 100,000 single nucleotide polymorphisms (SNPs) from Table A is present in a biological sample from the subject and calculating a polygenic risk score (PRS); wherein the presence of a risk allele of a SNP from Table A indicates that the subject has an increased risk of atrial fibrillation, and wherein the presence of an alternative allele indicates that the subject has a decreased risk of atrial fibrillation.

In an embodiment, the invention provides a method of determining a risk of developing atrial fibrillation in a subject comprising identifying whether the SNPs from Table A is present in a biological sample from the subject and calculating a polygenic risk score (PRS) for the subject based on the identified SNPs, wherein the PRS is calculated by summing the weighted risk score associated with each SNP identified. The number of identified SNPs can be at least 50, at least 95, at least 100, at least 200, at least 500, at least 1000, at least 2000, at least 5000, at least 10,000, at least 20,000, at least 50,000, at least 75,000, or at least 100,000.

In an of the embodiment, the invention provides a method of determining a risk of developing atrial fibrillation in a subject comprising identifying whether the SNPs from Table A is present in a biological sample from the subject, wherein identifying comprises measuring the presence of the at least 95 SNPs in the biological sample. The number of identified SNPs can be at least 50, at least 95, at least 100, at least 200, at least 500, at least 1000, at least 2000, at least 5000, at least 10,000, at least 20,000, at least 50,000, at least 75,000, or at least 100,000.

The invention provides a method of determining a polygenic risk score for (PRS) developing atrial fibrillation in a subject, the method comprising selecting at least 50, at least 95, at least 100, at least 200, at least 500, at least 1000, at least 2000, at least 5000, at least 10,000, at least 20,000, at least 50,000, at least 75,000, or at least 100,000 single nucleotide polymorphisms (SNPs) from Table A; identifying whether the SNPs are present in a biological sample from the subject; and calculating the polygenic risk score (PRS) based on the presence of the SNPs.

In an embodiment, the invention provides a method of determining a risk of developing atrial fibrillation in a subject comprising identifying whether the SNPs from Table A is present in a biological sample from the subject, calculating a polygenic risk score (PRS) for the subject based on the identified SNPs, and assigning the subject to a risk group based on the PRS. The PRS may be divided into quintiles, e.g., top quintile, intermediate quintile, and bottom quintile, wherein the top quintile of polygenic scores correspond the highest genetic risk group and the bottom quintile of polygenic scores correspond to the lowest genetic risk group. The number of identified SNPs can be at least 50, at least 95, at least 100, at least 200, at least 500, at least 1000, at least 2000, at least 5000, at least 10,000, at least 20,000, at least 50,000, at least 75,000, or at least 100,000.

In an embodiment, the invention provides a method for selecting subjects or candidates with a risk for developing atrial fibrillation comprising identifying whether at least 50, at least 95, at least 100, at least 200, at least 500, at least 1000, at least 2000, at least 5000, at least 10,000, at least 20,000, at least 50,000, at least 75,000, or at least 100,000 SNPs single nucleotide polymorphisms (SNPs) from Table A is present in a biological sample from each subject or candidate; calculating a polygenic risk score (PRS) for each subject or candidate based on the identified SNPs; and selecting the subjects or candidates with a desired risk group.

For all atrial fibrillation risk assessments, incorporation of large numbers of SNPs offers the advantage of increased predictive power. The invention further provides risk assessments outlined above incorporating for example, at least 500,000, at least 1,000,000, at least 2,000,000, at least 3,000,000, at least 4,000,000, at least 5,000,000, or at least 6,000,000 SNPs from Table A.

In certain embodiments of the invention, risk assessments comprise the highest weighted polymorphisms, including, but not limited to the top 50%, 55%, 60%, 70%, 80%, 90%, or 95% of SNPs from Table A.

In an embodiment, the method is used to select a population of subjects or candidates for clinical trials, e.g., a clinical trial to determine whether a particular treatment or treatment plan is effective against atrial fibrillation. In an embodiment, the desired risk group is a population comprising high risk subjects or candidates. In an embodiment, the selected population of subjects or candidates are responders, i.e., the subjects or candidates are responsive to the treatment or treatment plan.

In an embodiment, the invention provides a method for selecting a population of subjects or candidates with a high risk for developing atrial fibrillation comprising identifying whether at least 50, at least 95, at least 100, at least 200, at least 500, at least 1000, at least 2000, at least 5000, at least 10,000, at least 20,000, at least 50,000, at least 75,000, or at least 100,000 SNPs single nucleotide polymorphisms (SNPs) from Table A is present in a biological sample from each subject or candidate; calculating a polygenic risk score (PRS) for each subject or candidate based on the identified SNPs; and selecting the subjects or candidates in the high risk group. In an embodiment, the method is used to select a population of subjects or candidates for clinical trials, e.g., a clinical trial to determine whether a particular treatment or treatment plan is effective against atrial fibrillation. In an embodiment, the selected candidates or subjects are divided into subgroups based on the identified SNPs for each subject or candidate, and the method is used to determine whether a particular treatment or treatment plan is effective against a particular SNP or a particular group of SNPs. In other word, the method can be employed to determine susceptibility of a population of subjects to a particular treatment or treatment plan, wherein the population of subjects is selected based on the SNPs identified in the subjects.

In any of the above embodiment, the method may further comprise an initial step of obtaining a biological sample from the subject.

In any of the above embodiment, the number of identified SNPs is at least 100 SNPs.

In any of the above embodiment, the number of identified SNPs is at least 200 SNPs.

In any of the above embodiment, the number of identified SNPs is at least 500 SNPs.

In any of the above embodiment, the number of identified SNPs is at least 1,000 SNPs.

In any of the above embodiment, the number of identified SNPs is at least 2,000 SNPs.

In any of the above embodiment, the number of identified SNPs is at least 5,000 SNPs.

In any of the above embodiment, the number of identified SNPs is at least 10,000 SNPs.

In any of the above embodiment, the number of identified SNPs is at least 20,000 SNPs.

In any of the above embodiment, the number of identified SNPs is at least 50,000 SNPs.

In any of the above embodiment, the number of identified SNPs is at least 75,000 SNPs.

In any of the above embodiment, the number of identified SNPs is at least 100,000 SNPs.

In any of the above embodiment, the identified SNPs comprise the highest risk SNPs or SNPs with a weight risk score in the top 10%, top 20%, top 30%, top 40%, or top 50% in Table A.

In any of the above embodiments, the identified SNPs comprise one or more of rs17517928, rs2972146, rs17843797, rs748431, rs7623687, rs12493885, rs10857147, rs7678555, rs1800449, rs10841443, rs2244608, rs11057401, rs3851738, rs2972146, rs7500448, and rs8108632.

In any of the above embodiments, identifying whether the SNP is present includes obtaining information regarding the identity (i.e., of a specific nucleotide), presence or absence of one or more specific SNPs in a subject. Determining the presence of an SNP can, but need not, include obtaining a sample comprising DNA from a subject. The individual or organization who determines the presence of an SNP need not actually carry out the physical analysis of a sample from a subject; the methods can include using information obtained by analysis of the sample by a third party. Thus the methods can include steps that occur at more than one site. For example, a sample can be obtained from a subject at a first site, such as at a health care provider, or at the subject's home in the case of a self-testing kit. The sample can be analyzed at the same or a second site, e.g., at a laboratory or other testing facility. Identifying the presence of a SNP can be done by any DNA detection method known in the art, including sequencing at least part of a genome of one or more cells from the subject.

### SNP Detection

Sequencing can be, for example, whole genome sequencing. SNPs may be detected through hybridization-based methods, including dynamic allele-specific hybridization (DASH), molecular beacons, and SNP microarrays, enzyme-based methods including RFLP, PCR-based, e.g., allelic-specific polymerase chain reaction (AS-PCR), polymerase chain reaction—restriction fragment length polymorphism (PCR-RFLP), multiplex PCR real-time invader assay (mPCR-RETINA), (amplification refractory mutation system (ARMS), Flap endonuclease, primer extension, 5′ nuclease, e.g., Taqman or 5′nuclease allelic discrimination assay, and oligonucleotide ligation assay, and methods such as single strand conformation polymorphism, temperature gradient gel electrophoresis, denaturing high performance liquid chromatography, high-resolution melting of the entire amplicon, use of DNA mismatch-binding proteins, SNPlex, and Surveyor nuclease assay.

Sequencing can be, for example, whole genome sequencing. In certain embodiments, the invention involves plate based single cell RNA sequencing (see, e.g., Picelli, S. et al., 2014, “Full-length RNA-seq from single cells using Smart-seq2” Nature protocols 9, 171-181, doi:10.1038/nprot.2014.006). In certain embodiments, the invention involves high-throughput single-cell RNA-seq and/or targeted nucleic acid profiling (for example, sequencing, quantitative reverse transcription polymerase chain reaction, and the like) where the RNAs from different cells are tagged individually, allowing a single library to be created while retaining the cell identity of each read. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. January; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; and Gierahn et al., “Seq-Well: portable, low-cost RNA sequencing of single cells at high throughput” Nature Methods 14, 395-398 (2017), all the contents and disclosure of each of which are herein incorporated by reference in their entirety. In certain embodiments, the invention involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14(10):955-958; and International patent application number PCT/US2016/059239, published as WO2017164936 on Sep. 28, 2017, which are herein incorporated by reference in their entirety.

In certain example embodiments, target genomic regions of interest may be enriched from single cell sequencing libraries prior to sequencing analysis. Example enrichment methods are described, for example, in U.S. Provisional Application No. 62/576,031 entitled “Single Cell Cellular Component Enrichment from Barcoded Sequencing Libraries” filed Oct. 23, 2017.

Also disclosed herein are methods for detecting SNPs in a subject. In some cases, the method may include detecting whether one or more SNPs from Table A are present in a biological sample from subject. The detecting may include contacting the biological sample with a set of probes to each SNP, detecting binding other probes, amplifying genome regions comprising the SNPs using a set of amplification primers, sequencing genomic regions comprising or enriched for the SNPs, or any combination of these steps. In some cases, the method may detect whether at least 95 SNPs, at least 100 SNPs, at least 200 SNPs, or at least 500 SNPs, or at least 1000 SNPs, or at least 2000 SNPs, or at least 5000 SNPs are present in the biological sample.

### Methods of Treatment

In any of the above embodiments, the method may further comprises initiating a treatment to the subject. The treatment can be determined or adjusted according to the risk of atrial fibrillation or related diseases such as coronary artery disease or myocardial infarction. The treatment can comprise statins, ezetimibe, beta-blocking agents, angiotensin-converting-enzyme inhibitors, aspirin, anticoagulants, antiplatelet agents, angiotensin II receptor blockers, angiotensin receptor neprilysin inhibitors, calcium channel blockers, cholesterol-lowering medications, vasodilators, antidiuretics, renin-angiotensin system agents, lipid-modifying medicines, anti-inflammatory agents, nitrates, antiarrhythmic medicines, steroidal or non-steroidal anti-inflammatory drugs, DNA methyltransferase inhibitors and/or histone deacetylase inhibitors. The DNA methyltransferase inhibitors can be any DNA methyltransferase known in the art, e.g., 5-aza-2′-deoxycytidine or 5-azacytidine. The histone deacetylase inhibitors can be any histone deacetylase inhibitors known in the art, e.g., varinostat, romidepsin, panobinostat, belinostat or entinostat. The lipid-modifying medicines can be any lipid-modifying compounds known in the art, e.g., an antagonist of PCSK9, an antisense oligonucleotide targeting apolipoprotein C-III, and an antisense oligonucleotide to lower lipoprotein(a). The statins can be any statins known in the art, e.g., atorvastatin, fluvastatin, lovastatin, pravastatin, rosuvastatin, and simvastatin. Initiating a treatment can include devising a treatment plan based on the risk group, which corresponds to the PRS calculated for the subject.

In one embodiment, a treatment or a method of treatment can include gene therapy/genome editing and/or the nucleic acid vector used in a gene therapy vector known in the art. In one embodiment, one or more target locus within the subject's genomic DNA is targeted and modified. A treatment method comprises gene editing tools available in the art, e.g., CRISPR, zinc finger nucleases, meganucleases (Clustered Regularly Interspaced Short Palindromic Repeats), where a target DNA locus, e.g., a gene of interest, is modified to create a mutation in the gene product, e.g., a protein or enzyme, with reduced activity or no activity (loss-of-function mutation). In some embodiment, vectors can comprise viral vector, e.g., retroviruses, adenoviruses, adeno-associated viruses, and lentiviruses. Examples of a target locus of interest include the genes PCSK9, APOC3, ANGPTL8, LPL, CD36, HBB and NPC1L1.

The invention provides methods and models to establish causation of elements of alleles (e.g., chromosomal regions, genetic loci) identified as associated with increased disease risk. In an embodiment of the invention, a model animal, for example but not limited to a rat, a mouse, a dog, a pig, a non-human primate, or a chimeric animal comprising human cells can be employed. In an embodiment of the invention, an organ or organoid can be employed, which can be characterized as from a human or a non-human mammal. In an embodiment of the invention, a cell line from a human or non-human mammal can be employed.

The invention provides for modifying, for example mutating or modulating expression of, one or more genetic elements of a model. Such modifications can be made in a model organism singly, or in combination. In certain embodiments, a CRISPR system may be employed to mutate or regulate genetic elements singly or in combination in the organism. Thus by varying one or more genetic elements in a model organism, the invention provides a means for establishing or confirming causality between genetic changes and phenotypic effects. The genetic changes can be the SNPs or any variation in linkage disequilibrium with the SNP.

Similarly, the model organisms can be used to test effectiveness of therapeutic intervention. In an embodiment, the invention is used to define or establish subgroups of individuals (or models) at elevated risk for atrial fibrillation on the basis of different risk factors or combinations of risk factors. In one embodiment, the separate subgroups are used to characterize susceptibility to therapeutic interventions that may vary from subgroup to subgroup. In another embodiment, therapies are selected according the SNPs identified in a subject.

In an aspect of the invention, there is targeted genomic editing to modify one or more genomic sequences of interest to reduce disease risk. One or more targets may be selected, depending on the genotypic and/or phenotypic outcome. For instance, one or more therapeutic targets may be selected, depending on (genetic) disease etiology or the desired therapeutic outcome. The (therapeutic) target(s) may be a single gene, locus, or other genomic site, or may be multiple genes, loci or other genomic sites. As is known in the art, a single gene, locus, or other genomic site may be targeted more than once, such as by use of multiple gRNAs.

According to the invention, genomic sequences associated with disease risk are identified by single nucleotide polymorphisms (SNPs). The SNPs are linked to the genomic sequences of interest, i.e., close to or within the genomic sequences of interest, and may or may not be causative of the risk variation. That is, functional differences between alleles distinguished by the SNPs may result from sequence variation of an SNP or from one or more differences between alleles located near to the location of the SNP. In either case, the invention provides for gene editing in order to reduce disease risk. In general, a higher risk allele would be edited to resemble more closely a lower risk allele. Often such editing would involve individual base changes, but can also involve insertions and deletions. For example, trinucleotide repeat regions may be edited to change the number of trinucleotide repeats. In any of the above embodiment, the subject can be animal which include mammal, human and non-human mammal.

In an embodiment, the invention provides a method of identifying a risk of developing atrial fibrillation in a subject and providing a treatment to the subject, the method comprising obtaining a biological sample from the subject; identifying whether at least one single nucleotide polymorphism (SNP) from Table A is present in the biological sample; wherein the presence of a risk allele of a SNP from Table A indicates that the subject has an increased risk of atrial fibrillation; and initiating a treatment to the subject, wherein the treatment comprises statins, ezetimibe, beta-blocking agents, angiotensin-converting-enzyme inhibitors, aspirin, anticoagulants, antiplatelet agents, angiotensin II receptor blockers, angiotensin receptor neprilysin inhibitors, calcium channel blockers, cholesterol-lowering medications, vasodilators, antidiuretics, renin-angiotensin system agents, lipid-modifying medicines, anti-inflammatory agents, nitrates, antiarrhythmic medicines, steroidal or non-steroidal anti-inflammatory drugs, DNA methyltransferase inhibitors and/or histone deacetylase inhibitors.

In an embodiment, the invention provides a method of reducing a risk of atrial fibrillation in a subject comprising administering to the subject a treatment which comprises one or more statins, beta-blocking agents, angiotensin-converting-enzyme inhibitors, aspirin, anticoagulants, antiplatelet agents, angiotensin II receptor blockers, angiotensin receptor neprilysin inhibitors, calcium channel blockers, cholesterol-lowering medications, vasodilators, antidiuretics, renin-angiotensin system agents, lipid-modifying medicines, anti-inflammatory agents, nitrates, antiarrhythmic medicines, steroidal or non-steroidal anti-inflammatory drugs, DNA methyltransferase inhibitors and/or histone deacetylase inhibitors, wherein the subject has a polygenic risk score that corresponds to a high risk group. The polygenic risk score may be calculated by selecting at least 50, at least 95, at least 100, at least 200, at least 500, at least 1000, at least 2000, at least 5000, at least 10,000, at least 20,000, at least 50,000, at least 75,000, or at least 100,000 single nucleotide polymorphisms (SNPs) from Table A; identifying whether the at least 50, at least 95, at least 100, at least 200, at least 500, at least 1000, at least 2000, at least 5000, at least 10,000, at least 20,000, at least 50,000, at least 75,000, or at least 100,000, at least 500,000, at least 1,000,000, at least 2,000,000, at least 3,000,000, at least 4,000,000, at least 5,000,000, or at least 6,000,000 SNPs are present in a biological sample from the subject; and calculating the polygenic risk score (PRS) based on the presence of the SNPs.

Although the present invention and its advantages have been described in detail, it should be understood that various changes, substitutions and alterations can be made herein without departing from the spirit and scope of the invention as defined in the appended claims.

As used herein, the term “biological sample” is used in its broadest sense. A biological sample may be obtained from a subject (e.g., a human) or from components (e.g., tissues) of a subject. The sample may be of any biological tissue or fluid with which biomarkers of the present invention may be assayed. Frequently, the sample will be a “clinical sample”, i.e., a sample derived from a patient. Such samples include, but are not limited to, bodily fluids, e.g., urine, whole blood, blood plasma, saliva; tissue or fine needle biopsy samples; and archival samples with known diagnosis, treatment and/or outcome history. The term biological sample also encompasses any material derived by processing the biological sample. Derived materials include, but are not limited to, cells (or their progeny) isolated from the sample, proteins or nucleic acid molecules extracted from the sample. Processing of the biological sample may involve one or more of, filtration, distillation, extraction, concentration, inactivation of interfering components, addition of reagents, and the like. In some embodiments, the biological sample is a whole blood sample. In some embodiments, the biological sample includes peripheral blood mononuclear cells (PBMCs) obtained from a subject. PBMCs can be extracted from whole blood using ficoll, a hydrophilic polysaccharide that separates layers of blood, and gradient centrifugation, which will separate the blood into a top layer of plasma, followed by a layer of PBMCs and a bottom fraction of polymorphonuclear cells (such as neutrophils and eosinophils) and erythrocytes.

As used herein, an “allele” is one of a pair or series of genetic variants of a polymorphism at a specific genomic location. A “response allele” is an allele that is associated with altered response to a treatment. Where a SNP is biallelic, both alleles will be response alleles (e.g., one will be associated with a positive response, while the other allele is associated with no or a negative response, or some variation thereof).

As used herein, “genotype” refers to the diploid combination of alleles for a given genetic polymorphism. A homozygous subject carries two copies of the same allele and a heterozygous subject carries two different alleles.

As used herein, a “haplotype” is one or a set of signature genetic changes (polymorphisms) that are normally grouped closely together on the DNA strand, and are usually inherited as a group; the polymorphisms are also referred to herein as “markers.” A “haplotype” as used herein is information regarding the presence or absence of one or more genetic markers in a given chromosomal region in a subject. A haplotype can comprise a variety of genetic markers, including indels (insertions or deletions of the DNA at particular locations on the chromosome); single nucleotide polymorphisms (SNPs) in which a particular nucleotide is changed; microsatellites; and minis satellites.

The term “chromosome” as used herein refers to a gene carrier of a cell that is derived from chromatin and comprises DNA and protein components (e.g., histones). The conventional internationally recognized individual human genome chromosome numbering identification system is employed herein. The size of an individual chromosome can vary from one type to another with a given multi-chromosomal genome and from one genome to another. In the case of the human genome, the entire DNA mass of a given chromosome is usually greater than about 100,000,000 base pairs.

The term “gene” refers to a DNA sequence in a chromosome that codes for a product (either RNA or its translation product, a polypeptide). A gene contains a coding region and includes regions preceding and following the coding region (termed respectively “leader” and “trailer”). The coding region is comprised of a plurality of coding segments (“exons”) and intervening sequences (“introns”) between individual coding segments.

As used herein, the terms “protein”, “polypeptide”, and “peptide” are used herein interchangeably, and refer to amino acid sequences of a variety of lengths, either in their neutral (uncharged) forms or as salts, and either unmodified or modified by glycosylation, side chain oxidation, or phosphorylation, or modified by deletion, insertion, or change in one or more amino acids.

As used herein, the terms “nucleic acid molecule” and “polynucleotide” are used herein interchangeably. They refer to a deoxyribonucleotide or ribonucleotide polymer in either single- or double-stranded form, and unless otherwise stated, encompass known analogs of natural nucleotides that can function in a similar manner as naturally occurring nucleotides. The terms encompass nucleic acid-like structures with synthetic backbones, as well as amplification products.

As used herein, the term “hybridizing” refers to the binding of two single stranded nucleic acids via complementary base pairing. The term “specific hybridization” refers to a process in which a nucleic acid molecule preferentially binds, duplexes, or hybridizes to a particular nucleic acid sequence under stringent conditions (e.g., in the presence of competitor nucleic acids with a lower degree of complementarity to the hybridizing strand). In certain embodiments of the present invention, these terms more specifically refer to a process in which a nucleic acid fragment (or segment) from a test sample preferentially binds to a particular probe and to a lesser extent or not at all, to other probes, for example, when these probes are immobilized on an array.

The term “probe” refers to an oligonucleotide. A probe can be single stranded at the time of hybridization to a target. As used herein, probes include primers, i.e., oligonucleotides that can be used to prime a reaction, e.g., a PCR reaction.

The term “label” or “label containing moiety” refers in a moiety capable of detection, such as a radioactive isotope or group containing same, and nonisotopic labels, such as enzymes, biotin, avidin, streptavidin, digoxygenin, luminescent agents, dyes, haptens, and the like. Luminescent agents, depending upon the source of exciting energy, can be classified as radioluminescent, chemiluminescent, bioluminescent, and photoluminescent (including fluorescent and phosphorescent). A probe described herein can be bound, e.g., chemically bound to label-containing moieties or can be suitable to be so bound. The probe can be directly or indirectly labeled.

The term “direct label probe” (or “directly labeled probe”) refers to a nucleic acid probe whose label after hybrid formation with a target is detectable without further reactive processing of hybrid. The term “indirect label probe” (or “indirectly labeled probe”) refers to a nucleic acid probe whose label after hybrid formation with a target is further reacted in subsequent processing with one or more reagents to associate therewith one or more moieties that finally result in a detectable entity.

The terms “target,” “DNA target,” or “DNA target locus” refers to a nucleotide sequence that occurs at a specific chromosomal location. Each such sequence or portion is preferably at least partially, single stranded (e.g., denatured) at the time of hybridization. When the target nucleotide sequences are located only in a single region or fraction of a given chromosome, the term “target region” is sometimes used. Targets for hybridization can be derived from specimens which include, but are not limited to, chromosomes or regions of chromosomes in normal, diseased or malignant human cells, either interphase or at any state of meiosis or mitosis, and either extracted or derived from living or postmortem tissues, organs or fluids; germinal cells including sperm and egg cells, or cells from zygotes, fetuses, or embryos, or chorionic or amniotic cells, or cells from any other germinating body; cells grown in vitro, from either long-term or short-term culture, and either normal, immortalized or transformed; inter- or intraspecific hybrids of different types of cells or differentiation states of these cells; individual chromosomes or portions of chromosomes, or translocated, deleted or other damaged chromosomes, isolated by any of a number of means known to those with skill in the art, including libraries of such chromosomes cloned and propagated in prokaryotic or other cloning vectors, or amplified in vitro by means well known to those with skill; or any forensic material, including but not limited to blood, or other samples.

As used herein, the terms “array”, “micro-array”, and “biochip” are used herein interchangeably. They refer to an arrangement, on a substrate surface, of hybridizable array elements, preferably, multiple nucleic acid molecules of known sequences. Each nucleic acid molecule is immobilized to a discrete spot (i.e., a defined location or assigned position) on the substrate surface. The term “micro-array” more specifically refers to an array that is miniaturized so as to require microscopic examination for visual evaluation.

### Nucleases and Related Systems

The treatment may include administering one or more genetic modifying agents. In some embodiments, the genetic modifying agents may be nucleases or related systems. The genetic modifying agents may also be used to make one or more genetic modifications in a model organism. In certain example embodiments, one or more genetic elements in the model organism may be modified using a nuclease. The term “nuclease” as used herein broadly refers to an agent, for example a protein or a small molecule, capable of cleaving a phosphodiester bond connecting nucleotide residues in a nucleic acid molecule. In some embodiments, a nuclease may be a protein, e.g., an enzyme that can bind a nucleic acid molecule and cleave a phosphodiester bond connecting nucleotide residues within the nucleic acid molecule. A nuclease may be an endonuclease, cleaving a phosphodiester bonds within a polynucleotide chain, or an exonuclease, cleaving a phosphodiester bond at the end of the polynucleotide chain. Preferably, the nuclease is an endonuclease. Preferably, the nuclease is a site-specific nuclease, binding and/or cleaving a specific phosphodiester bond within a specific nucleotide sequence, which may be referred to as “recognition sequence”, “nuclease target site”, or “target site”. In some embodiments, a nuclease may recognize a single stranded target site, in other embodiments a nuclease may recognize a double-stranded target site, for example a double-stranded DNA target site. Some endonucleases cut a double-stranded nucleic acid target site symmetrically, i.e., cutting both strands at the same position so that the ends comprise base-paired nucleotides, also known as blunt ends. Other endonucleases cut a double-stranded nucleic acid target sites asymmetrically, i.e., cutting each strand at a different position so that the ends comprise unpaired nucleotides. Unpaired nucleotides at the end of a double-stranded DNA molecule are also referred to as “overhangs”, e.g., “5′-overhang” or “3′-overhang”, depending on whether the unpaired nucleotide(s) form(s) the 5′ or the 5′ end of the respective DNA strand.

The nuclease may introduce one or more single-strand nicks and/or double-strand breaks in the endogenous gene, whereupon the sequence of the endogenous gene may be modified or mutated via non-homologous end joining (NHEJ) or homology-directed repair (HDR).

In certain embodiments, the nuclease may comprise (i) a DNA-binding portion configured to specifically bind to the endogenous gene and (ii) a DNA cleavage portion. Generally, the DNA cleavage portion will cleave the nucleic acid within or in the vicinity of the sequence to which the DNA-binding portion is configured to bind.

In certain embodiments, the nuclease may be employed to mutate or regulate genetic elements singly or in combination in the organism. Thus by varying one or more genetic elements in a model organism, the invention provides a means for establishing or confirming causality between genetic changes and phenotypic effects. The genetic changes can be the SNPs or any variation in linkage disequilibrium with the SNP.

Similarly, the model organisms can be used to test effectiveness of therapeutic intervention. In an embodiment, the invention is used to define or establish subgroups of individuals (or models) at elevated risk for coronary artery disease on the basis of different risk factors or combinations of risk factors. In one embodiment, the separate subgroups are used to characterize susceptibility to therapeutic interventions that may vary from subgroup to subgroup. In another embodiment, therapies are selected according the SNPs identified in a subject.

In an aspect of the invention, there is targeted genomic editing to modify one or more genomic sequences of interest to reduce disease risk. One or more targets may be selected, depending on the genotypic and/or phenotypic outcome. For instance, one or more therapeutic targets may be selected, depending on (genetic) disease etiology or the desired therapeutic outcome. The (therapeutic) target(s) may be a single gene, locus, or other genomic site, or may be multiple genes, loci or other genomic sites. As is known in the art, a single gene, locus, or other genomic site may be targeted more than once, such as by use of multiple gRNAs.

In certain embodiments, the nuclease is used for gene editing. Nuclease based therapy or therapeutics may involve target disruption, such as target mutation, such as leading to gene knockout. Nuclease activity, such as CRISPR-Cas system based therapy or therapeutics may involve replacement of particular target sites, such as leading to target correction. Nuclease based therapy or therapeutics may involve removal of particular target sites, such as leading to target deletion. Nuclease activity, such as CRISPR-Cas system based therapy or therapeutics may involve modulation of target site functionality, such as target site activity or accessibility, leading for instance to (transcriptional and/or epigenetic) gene or genomic region activation or gene or genomic region silencing. The skilled person will understand that modulation of target site functionality may involve nuclease mutation (such as for instance generation of a catalytically inactive CRISPR effector) and/or functionalization (such as for instance fusion of the CRISPR effector with a heterologous functional domain, such as a transcriptional activator or repressor), as described herein elsewhere.

Accordingly, in an aspect, the invention relates to a method as described herein, comprising selection of one or more (therapeutic) target, selecting one or more nuclease function, and optimization of selected parameters or variables associated with the nuclease system and/or its functionality. In a related aspect, the invention relates to a method as described herein, comprising (a) selecting one or more (therapeutic) target loci, (b) selecting one or more nuclease system functionalities, (c) optionally selecting one or more modes of delivery, and preparing, developing, or designing a CRISPR-Cas system selected based on steps (a)-(c). Method for selecting optimal Cas9 and Cas12 based systems are disclosed, for example, in International Patent Application Publication Nos. WO/2018/035388 and WO/2018/035387.

In certain embodiments, nuclease system functionality comprises genomic mutation. In certain embodiments, nuclease system functionality comprises single genomic mutation. In certain embodiments, nuclease system functionality comprises multiple genomic mutations. In certain embodiments, nuclease system functionality comprises gene knockout. In certain embodiments, nuclease system functionality comprises single gene knockout. In certain embodiments, nuclease system functionality comprises multiple gene knockout. In certain embodiments, nuclease system functionality comprises gene correction. In certain embodiments, nuclease system functionality comprises single gene correction. In certain embodiments, nuclease system functionality comprises multiple gene correction. In certain embodiments, nuclease system functionality comprises genomic region correction. In certain embodiments, nuclease system functionality comprises single genomic region correction. In certain embodiments, nuclease system functionality comprises multiple genomic region correction. In certain embodiments, nuclease system functionality comprises gene deletion. In certain embodiments, nuclease system functionality comprises single gene deletion. In certain embodiments, nuclease system functionality comprises multiple gene deletion. In certain embodiments, nuclease system functionality comprises genomic region deletion. In certain embodiments, nuclease system functionality comprises single genomic region deletion. In certain embodiments, nuclease system functionality comprises multiple genomic region deletion. In certain embodiments, nuclease system functionality comprises modulation of gene or genomic region functionality. In certain embodiments, nuclease system functionality comprises modulation of single gene or genomic region functionality. In certain embodiments, nuclease system functionality comprises modulation of multiple gene or genomic region functionality. In certain embodiments, nuclease system functionality comprises gene or genomic region functionality, such as gene or genomic region activity. In certain embodiments, nuclease system functionality comprises single gene or genomic region functionality, such as gene or genomic region activity. In certain embodiments, nuclease system functionality comprises multiple gene or genomic region functionality, such as gene or genomic region activity. In certain embodiments, nuclease system functionality comprises modulation gene activity or accessibility optionally leading to transcriptional and/or epigenetic gene or genomic region activation or gene or genomic region silencing. In certain embodiments, nuclease system functionality comprises modulation single gene activity or accessibility optionally leading to transcriptional and/or epigenetic gene or genomic region activation or gene or genomic region silencing. In certain embodiments, nuclease system functionality comprises modulation multiple gene activity or accessibility optionally leading to transcriptional and/or epigenetic gene or genomic region activation or gene or genomic region silencing.

Accordingly, in an aspect, the invention relates to a method as described herein, comprising selection of one or more (therapeutic) target, selecting nuclease system functionality, selecting nuclease system mode of delivery, and optimization of selected parameters or variables associated with the nuclease system and/or its functionality.

### Exemplary Genetic Modifying Agents

The genetic modifying agents may be programmable nucleic acid-modifying agents, which may be used to modify endogenous cell DNA or RNA sequences, including DNA and/or RNA sequences encoding the target genes and target gene products disclosed herein. In certain example embodiments, the programmable nucleic acid-modifying agents may be used to edit a target sequence to restore native or wild-type functionality. In certain other embodiments, the programmable nucleic-acid modifying agents may be used to insert a new gene or gene product to modify the phenotype of target cells. In certain other example embodiments, the programmable nucleic-acid modifying agents may be used to delete or otherwise silence the expression of a target gene or gene product. Programmable nucleic-acid modifying agents may be used in both in vivo an ex vivo applications disclosed herein.

Examples of genetic modifying agents are described below.

### CRISPR/Cas Systems

In certain embodiments, the genetic modifying agents may be a CRISPR-Cas system or one or more components thereof. CRISPR-Cas system activity, such as CRISPR-Cas system based therapy or therapeutics may involve target disruption, such as target mutation, such as leading to gene knockout. CRISPR-Cas system activity, such as CRISPR-Cas system based therapy or therapeutics may involve replacement of particular target sites, such as leading to target correction. CRISPR-Cas system based therapy or therapeutics may involve removal of particular target sites, such as leading to target deletion. CRISPR-Cas system activity, such as CRISPR-Cas system based therapy or therapeutics may involve modulation of target site functionality, such as target site activity or accessibility, leading for instance to (transcriptional and/or epigenetic) gene or genomic region activation or gene or genomic region silencing. The skilled person will understand that modulation of target site functionality may involve CRISPR effector mutation (such as for instance generation of a catalytically inactive CRISPR effector) and/or functionalization (such as for instance fusion of the CRISPR effector with a heterologous functional domain, such as a transcriptional activator or repressor), as described herein elsewhere.

Accordingly, in an aspect, the invention relates to a method as described herein, comprising selection of one or more (therapeutic) target, selecting one or more CRISPR-Cas system functionality, and optimization of selected parameters or variables associated with the CRISPR-Cas system and/or its functionality. In a related aspect, the invention relates to a method as described herein, comprising (a) selecting one or more (therapeutic) target loci, (b) selecting one or more CRISPR-Cas system functionalities, (c) optionally selecting one or more modes of delivery, and preparing, developing, or designing a CRISPR-Cas system selected based on steps (a)-(c).

In certain embodiments, CRISPR-Cas system functionality comprises genomic mutation. In certain embodiments, CRISPR-Cas system functionality comprises single genomic mutation. In certain embodiments, CRISPR-Cas system functionality comprises multiple genomic mutations. In certain embodiments, CRISPR-Cas system functionality comprises gene knockout. In certain embodiments, CRISPR-Cas system functionality comprises single gene knockout. In certain embodiments, CRISPR-Cas system functionality comprises multiple gene knockout. In certain embodiments, CRISPR-Cas system functionality comprises gene correction. In certain embodiments, CRISPR-Cas system functionality comprises single gene correction. In certain embodiments, CRISPR-Cas system functionality comprises multiple gene correction. In certain embodiments, CRISPR-Cas system functionality comprises genomic region correction. In certain embodiments, CRISPR-Cas system functionality comprises single genomic region correction. In certain embodiments, CRISPR-Cas system functionality comprises multiple genomic region correction. In certain embodiments, CRISPR-Cas system functionality comprises gene deletion. In certain embodiments, CRISPR-Cas system functionality comprises single gene deletion. In certain embodiments, CRISPR-Cas system functionality comprises multiple gene deletion. In certain embodiments, CRISPR-Cas system functionality comprises genomic region deletion. In certain embodiments, CRISPR-Cas system functionality comprises single genomic region deletion. In certain embodiments, CRISPR-Cas system functionality comprises multiple genomic region deletion. In certain embodiments, CRISPR-Cas system functionality comprises modulation of gene or genomic region functionality. In certain embodiments, CRISPR-Cas system functionality comprises modulation of single gene or genomic region functionality. In certain embodiments, CRISPR-Cas system functionality comprises modulation of multiple gene or genomic region functionality. In certain embodiments, CRISPR-Cas system functionality comprises gene or genomic region functionality, such as gene or genomic region activity. In certain embodiments, CRISPR-Cas system functionality comprises single gene or genomic region functionality, such as gene or genomic region activity. In certain embodiments, CRISPR-Cas system functionality comprises multiple gene or genomic region functionality, such as gene or genomic region activity. In certain embodiments, CRISPR-Cas system functionality comprises modulation gene activity or accessibility optionally leading to transcriptional and/or epigenetic gene or genomic region activation or gene or genomic region silencing. In certain embodiments, CRISPR-Cas system functionality comprises modulation single gene activity or accessibility optionally leading to transcriptional and/or epigenetic gene or genomic region activation or gene or genomic region silencing. In certain embodiments, CRISPR-Cas system functionality comprises modulation multiple gene activity or accessibility optionally leading to transcriptional and/or epigenetic gene or genomic region activation or gene or genomic region silencing.

The methods as described herein may further involve selection of the CRISPR-Cas system mode of delivery. In certain embodiments, gRNA (and tracr, if and where needed, optionally provided as a sgRNA) and/or CRISPR effector protein are or are to be delivered. In certain embodiments, gRNA (and tracr, if and where needed, optionally provided as a sgRNA) and/or CRISPR effector mRNA are or are to be delivered. In certain embodiments, gRNA (and tracr, if and where needed, optionally provided as a sgRNA) and/or CRISPR effector provided in a DNA-based expression system are or are to be delivered. In certain embodiments, delivery of the individual CRISPR-Cas system components comprises a combination of the above modes of delivery. In certain embodiments, delivery comprises delivering gRNA and/or CRISPR effector protein, delivering gRNA and/or CRISPR effector mRNA, or delivering gRNA and/or CRISPR effector as a DNA based expression system.

Accordingly, in an aspect, the invention relates to a method as described herein, comprising selection of one or more (therapeutic) target, selecting CRISPR-Cas system functionality, selecting CRISPR-Cas system mode of delivery, and optimization of selected parameters or variables associated with the CRISPR-Cas system and/or its functionality.

The methods as described herein may further involve selection of the CRISPR-Cas system delivery vehicle and/or expression system. Delivery vehicles and expression systems are described herein elsewhere. By means of example, delivery vehicles of nucleic acids and/or proteins include nanoparticles, liposomes, etc. Delivery vehicles for DNA, such as DNA-based expression systems include for instance biolistics, viral based vector systems (e.g. adenoviral, AAV, lentiviral), etc. the skilled person will understand that selection of the mode of delivery, as well as delivery vehicle or expression system may depend on for instance the cell or tissues to be targeted. In certain embodiments, the delivery vehicle and/or expression system for delivering the CRISPR-Cas systems or components thereof comprises liposomes, lipid particles, nanoparticles, biolistics, or viral-based expression/delivery systems.

Optimization of selected parameters or variables in the methods as described herein may result in optimized or improved nuclease system, such as CRISPR-Cas system based therapy or therapeutic, specificity, efficacy, and/or safety. In certain embodiments, one or more of the following parameters or variables are taken into account, are selected, or are optimized in the methods of the invention as described herein: CRISPR effector specificity, gRNA specificity, CRISPR-Cas complex specificity, PAM restrictiveness, PAM type (natural or modified), PAM nucleotide content, PAM length, CRISPR effector activity, gRNA activity, CRISPR-Cas complex activity, target cleavage efficiency, target site selection, target sequence length, ability of effector protein to access regions of high chromatin accessibility, degree of uniform enzyme activity across genomic targets, epigenetic tolerance, mismatch/budge tolerance, CRISPR effector stability, CRISPR effector mRNA stability, gRNA stability, CRISPR-Cas complex stability, CRISPR effector protein or mRNA immunogenicity or toxicity, gRNA immunogenicity or toxicity, CRISPR-Cas complex immunogenicity or toxicity, CRISPR effector protein or mRNA dose or titer, gRNA dose or titer, CRISPR-Cas complex dose or titer, CRISPR effector protein size, CRISPR effector expression level, gRNA expression level, CRISPR-Cas complex expression level, CRISPR effector spatiotemporal expression, gRNA spatiotemporal expression, CRISPR-Cas complex spatiotemporal expression.

In certain embodiments, selecting one or more CRISPR-Cas system functionalities comprises selecting one or more of an optimal effector protein, an optimal guide RNA, or both.

In an exemplary method for modifying a target polynucleotide by integrating an exogenous polynucleotide template, a double stranded break is introduced into the genome sequence by the CRISPR complex, the break is repaired via homologous recombination an exogenous polynucleotide template such that the template is integrated into the genome. The presence of a double-stranded break facilitates integration of the template.

In an exemplary method for modifying a target polynucleotide by integrating an exogenous polynucleotide template, a single stranded break is introduced into the genome sequence by the CRISPR complex, for example wherein the CRISPR-Cas protein is a nickase. The break is repaired via homologous recombination an exogenous polynucleotide template such that the template is integrated into the genome. The presence of a single-stranded break facilitates integration of the template.

In certain embodiments, the therapeutic CRISPR system is multiplexed for targeting multiple loci. In certain embodiments, this can be established by using multiple (tandem or multiplex) guide RNA (gRNA) sequences. In certain embodiments, said gRNA sequences are separated by a nucleotide sequence, such as a direct repeat (DR). In certain embodiments, said gRNA sequences are separated by a sequence cleavable by a host enzyme. In certain embodiments, a “self-inactivating” gRNA includes which targets an element of the CRISPR system.

In certain embodiments, selecting an optimal effector protein comprises optimizing one or more of effector protein type, size, PAM specificity, effector protein stability, immunogenicity or toxicity, functional specificity, and efficacy, or other CRISPR effector associated parameters or variables as described herein elsewhere.

The invention further provides for targeted delivery whereby a CRISPR system is preferably delivered to a cell type of interest. In one embodiment, it may be preferable for a CRISPR system engineered to target certain genetic loci to a particular cell type wherein those loci are expressed and active. According to the invention, a CRISPR system can be preferentially targeted to, without limitation, to a liver cell, an epithelial cell, a hematopoietic cell, or an immune cell. In an embodiment of the invention, a cell type of interest is preferentially targeted by using viral vectors of a particular serotypes. In an embodiment of the invention, a cell type of interest is preferentially targeted by a vector particle displaying a target-specific ligand.

In general, a CRISPR-Cas or CRISPR system as used herein and in documents, such as WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g., Shmakov et al. (2015) “Discovery and Functional Characterization of Diverse Class 2 CRISPR-Cas Systems”, Molecular Cell, DOI: dx.doi.org/10.1016/j.molcel.2015.10.008.

In certain embodiments, a protospacer adjacent motif (PAM) or PAM-like motif directs binding of the effector protein complex as disclosed herein to the target locus of interest. In some embodiments, the PAM may be a 5′ PAM (i.e., located upstream of the 5′ end of the protospacer). In other embodiments, the PAM may be a 3′ PAM (i.e., located downstream of the 5′ end of the protospacer). The term “PAM” may be used interchangeably with the term “PFS” or “protospacer flanking site” or “protospacer flanking sequence”.

In a preferred embodiment, the CRISPR effector protein may recognize a 3′ PAM. In certain embodiments, the CRISPR effector protein may recognize a 3′ PAM which is 5′H, wherein H is A, C or U.

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to a RNA polynucleotide being or comprising the target sequence. In other words, the target RNA may be a RNA polynucleotide or a part of a RNA polynucleotide to which a part of the gRNA, i.e. the guide sequence, is designed to have complementarity and to which the effector function mediated by the complex comprising CRISPR effector protein and a gRNA is to be directed. In some embodiments, a target sequence is located in the nucleus or cytoplasm of a cell.

In certain example embodiments, the CRISPR effector protein may be delivered using a nucleic acid molecule encoding the CRISPR effector protein. The nucleic acid molecule encoding a CRISPR effector protein, may advantageously be a codon optimized CRISPR effector protein. An example of a codon optimized sequence, is in this instance a sequence optimized for expression in eukaryote, e.g., humans (i.e. being optimized for expression in humans), or for another eukaryote, animal or mammal as herein discussed; see, e.g., SaCas9 human codon optimized sequence in WO 2014/093622 (PCT/US2013/074667). Whilst this is preferred, it will be appreciated that other examples are possible and codon optimization for a host species other than human, or for codon optimization for specific organs is known. In some embodiments, an enzyme coding sequence encoding a CRISPR effector protein is a codon optimized for expression in particular cells, such as eukaryotic cells. The eukaryotic cells may be those of or derived from a particular organism, such as a plant or a mammal, including but not limited to human, or non-human eukaryote or animal or mammal as herein discussed, e.g., mouse, rat, rabbit, dog, livestock, or non-human mammal or primate. In some embodiments, processes for modifying the germ line genetic identity of human beings and/or processes for modifying the genetic identity of animals which are likely to cause them suffering without any substantial medical benefit to man or animal, and also animals resulting from such processes, may be excluded. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at kazusa.orjp/codon/ and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding a Cas correspond to the most frequently used codon for a particular amino acid.

In certain embodiments, the methods as described herein may comprise providing a Cas transgenic cell in which one or more nucleic acids encoding one or more guide RNAs are provided or introduced operably connected in the cell with a regulatory element comprising a promoter of one or more gene of interest. As used herein, the term “Cas transgenic cell” refers to a cell, such as a eukaryotic cell, in which a Cas gene has been genomically integrated. The nature, type, or origin of the cell are not particularly limiting according to the present invention. Also the way the Cas transgene is introduced in the cell may vary and can be any method as is known in the art. In certain embodiments, the Cas transgenic cell is obtained by introducing the Cas transgene in an isolated cell. In certain other embodiments, the Cas transgenic cell is obtained by isolating cells from a Cas transgenic organism. By means of example, and without limitation, the Cas transgenic cell as referred to herein may be derived from a Cas transgenic eukaryote, such as a Cas knock-in eukaryote. Reference is made to WO 2014/093622 (PCT/US13/74667), incorporated herein by reference. Methods of US Patent Publication Nos. 20120017290 and 20110265198 assigned to Sangamo BioSciences, Inc. directed to targeting the Rosa locus may be modified to utilize the CRISPR Cas system of the present invention. Methods of US Patent Publication No. 20130236946 assigned to Cellectis directed to targeting the Rosa locus may also be modified to utilize the CRISPR Cas system of the present invention. By means of further example reference is made to Platt et. al. (Cell; 159(2):440-455 (2014)), describing a Cas9 knock-in mouse, which is incorporated herein by reference. The Cas transgene can further comprise a Lox-Stop-polyA-Lox (LSL) cassette thereby rendering Cas expression inducible by Cre recombinase. Alternatively, the Cas transgenic cell may be obtained by introducing the Cas transgene in an isolated cell. Delivery systems for transgenes are well known in the art. By means of example, the Cas transgene may be delivered in for instance eukaryotic cell by means of vector (e.g., AAV, adenovirus, lentivirus) and/or particle and/or nanoparticle delivery, as also described herein elsewhere.

It will be understood by the skilled person that the cell, such as the Cas transgenic cell, as referred to herein may comprise further genomic alterations besides having an integrated Cas gene or the mutations arising from the sequence specific action of Cas when complexed with RNA capable of guiding Cas to a target locus.

In certain aspects the invention involves vectors, e.g. for delivering or introducing in a cell Cas and/or RNA capable of guiding Cas to a target locus (i.e. guide RNA), but also for propagating these components (e.g. in prokaryotic cells). A used herein, a “vector” is a tool that allows or facilitates the transfer of an entity from one environment to another. It is a replicon, such as a plasmid, phage, or cosmid, into which another DNA segment may be inserted so as to bring about the replication of the inserted segment. Generally, a vector is capable of replication when associated with the proper control elements. In general, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell). With regards to recombination and cloning methods, mention is made of U.S. patent application Ser. No. 10/815,730, published Sep. 2, 2004 as US 2004-0171156 A1, the contents of which are herein incorporated by reference in their entirety. Thus, the embodiments disclosed herein may also comprise transgenic cells comprising the CRISPR effector system. In certain example embodiments, the transgenic cell may function as an individual discrete volume. In other words samples comprising a masking construct may be delivered to a cell, for example in a suitable delivery vesicle and if the target is present in the delivery vesicle the CRISPR effector is activated and a detectable signal generated.

The vector(s) can include the regulatory element(s), e.g., promoter(s). The vector(s) can comprise Cas encoding sequences, and/or a single, but possibly also can comprise at least 3 or 8 or 16 or 32 or 48 or 50 guide RNA(s) (e.g., sgRNAs) encoding sequences, such as 1-2, 1-3, 1-4 1-5, 3-6, 3-7, 3-8, 3-9, 3-10, 3-8, 3-16, 3-30, 3-32, 3-48, 3-50 RNA(s) (e.g., sgRNAs). In a single vector there can be a promoter for each RNA (e.g., sgRNA), advantageously when there are up to about 16 RNA(s); and, when a single vector provides for more than 16 RNA(s), one or more promoter(s) can drive expression of more than one of the RNA(s), e.g., when there are 32 RNA(s), each promoter can drive expression of two RNA(s), and when there are 48 RNA(s), each promoter can drive expression of three RNA(s). By simple arithmetic and well established cloning protocols and the teachings in this disclosure one skilled in the art can readily practice the invention as to the RNA(s) for a suitable exemplary vector such as AAV, and a suitable promoter such as the U6 promoter. For example, the packaging limit of AAV is ˜4.7 kb. The length of a single U6-gRNA (plus restriction sites for cloning) is 361 bp. Therefore, the skilled person can readily fit about 12-16, e.g., 13 U6-gRNA cassettes in a single vector. This can be assembled by any suitable means, such as a golden gate strategy used for TALE assembly (genome-engineering.org/taleffectors/). The skilled person can also use a tandem guide strategy to increase the number of U6-gRNAs by approximately 1.5 times, e.g., to increase from 12-16, e.g., 13 to approximately 18-24, e.g., about 19 U6-gRNAs. Therefore, one skilled in the art can readily reach approximately 18-24, e.g., about 19 promoter-RNAs, e.g., U6-gRNAs in a single vector, e.g., an AAV vector. A further means for increasing the number of promoters and RNAs in a vector is to use a single promoter (e.g., U6) to express an array of RNAs separated by cleavable sequences. And an even further means for increasing the number of promoter-RNAs in a vector, is to express an array of promoter-RNAs separated by cleavable sequences in the intron of a coding sequence or gene; and, in this instance it is advantageous to use a polymerase II promoter, which can have increased expression and enable the transcription of long RNA in a tissue specific manner. (see, e.g., nar.oxfordjournals.org/content/34/7/e53.short and nature.com/mt/journal/v16/n9/abs/mt2008144a.html). In an advantageous embodiment, AAV may package U6 tandem gRNA targeting up to about 50 genes. Accordingly, from the knowledge in the art and the teachings in this disclosure the skilled person can readily make and use vector(s), e.g., a single vector, expressing multiple RNAs or guides under the control or operatively or functionally linked to one or more promoters-especially as to the numbers of RNAs or guides discussed herein, without any undue experimentation.

The guide RNA(s) encoding sequences and/or Cas encoding sequences, can be functionally or operatively linked to regulatory element(s) and hence the regulatory element(s) drive expression. The promoter(s) can be constitutive promoter(s) and/or conditional promoter(s) and/or inducible promoter(s) and/or tissue specific promoter(s). The promoter can be selected from the group consisting of RNA polymerases, pol I, pol II, pol III, T7, U6, H1, retroviral Rous sarcoma virus (RSV) LTR promoter, the cytomegalovirus (CMV) promoter, the SV40 promoter, the dihydrofolate reductase promoter, the (3-actin promoter, the phosphoglycerol kinase (PGK) promoter, and the EF1α promoter. An advantageous promoter is the promoter is U6.

Additional effectors for use according to the invention can be identified by their proximity to cas1 genes, for example, though not limited to, within the region 20 kb from the start of the cas1 gene and 20 kb from the end of the cas1 gene. In certain embodiments, the effector protein comprises at least one HEPN domain and at least 500 amino acids, and wherein the C2c2 effector protein is naturally present in a prokaryotic genome within 20 kb upstream or downstream of a Cas gene or a CRISPR array. Examples of Cas proteins include those of Class 1 (e.g., Type I, Type III, and Type IV) and Class 2 (e.g., Type II, Type V, and Type VI) Cas proteins, e.g., Cas9, Cas12 (e.g., Cas12a, Cas12b, Cas12c, Cas12d), Cas13 (e.g., Cas13a, Cas13b, Cas13c, Cas13d,), CasX, CasY, Cas14, variants thereof (e.g., mutated forms, truncated forms), homologs thereof, and orthologs thereof. In some examples, the Cas effector protein is Cas9. In some examples, the Cas effector protein is Cas12. In some examples, the Cas effector protein is Cas13. Additional non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified versions thereof. In certain example embodiments, the C2c2 effector protein is naturally present in a prokaryotic genome within 20 kb upstream or downstream of a Cas 1 gene. The terms “orthologue” (also referred to as “ortholog” herein) and “homologue” (also referred to as “homolog” herein) are well known in the art. By means of further guidance, a “homologue” of a protein as used herein is a protein of the same species which performs the same or a similar function as the protein it is a homologue of. Homologous proteins may but need not be structurally related, or are only partially structurally related. An “orthologue” of a protein as used herein is a protein of a different species which performs the same or a similar function as the protein it is an orthologue of. Orthologous proteins may but need not be structurally related, or are only partially structurally related.

The methods as described herein may further involve selection of the nuclease system mode of delivery. In certain embodiments, gRNA (and tracr, if and where needed, optionally provided as a sgRNA) and/or CRISPR effector protein are or are to be delivered. In certain embodiments, gRNA (and tracr, if and where needed, optionally provided as a sgRNA) and/or CRISPR effector mRNA are or are to be delivered. In certain embodiments, gRNA (and tracr, if and where needed, optionally provided as a sgRNA) and/or CRISPR effector provided in a DNA-based expression system are or are to be delivered. In certain embodiments, delivery of the individual CRISPR-Cas system components comprises a combination of the above modes of delivery. In certain embodiments, delivery comprises delivering gRNA and/or CRISPR effector protein, delivering gRNA and/or CRISPR effector mRNA, or delivering gRNA and/or CRISPR effector as a DNA based expression system.

### DNA Repair and NHEJ

In certain embodiments, nuclease-induced non-homologous end-joining (NHEJ) can be used to target gene-specific knockouts. Nuclease-induced NHEJ can also be used to remove (e.g., delete) sequence in a gene of interest. Generally, NHEJ repairs a double-strand break in the DNA by joining together the two ends; however, generally, the original sequence is restored only if two compatible ends, exactly as they were formed by the double-strand break, are perfectly ligated. The DNA ends of the double-strand break are frequently the subject of enzymatic processing, resulting in the addition or removal of nucleotides, at one or both strands, prior to rejoining of the ends. This results in the presence of insertion and/or deletion (indel) mutations in the DNA sequence at the site of the NHEJ repair. Two-thirds of these mutations typically alter the reading frame and, therefore, produce a non-functional protein. Additionally, mutations that maintain the reading frame, but which insert or delete a significant amount of sequence, can destroy functionality of the protein. This is locus dependent as mutations in critical functional domains are likely less tolerable than mutations in non-critical regions of the protein. The indel mutations generated by NHEJ are unpredictable in nature; however, at a given break site certain indel sequences are favored and are over represented in the population, likely due to small regions of microhomology. The lengths of deletions can vary widely; most commonly in the 1-50 bp range, but they can easily be greater than 50 bp, e.g., they can easily reach greater than about 100-200 bp. Insertions tend to be shorter and often include short duplications of the sequence immediately surrounding the break site. However, it is possible to obtain large insertions, and in these cases, the inserted sequence has often been traced to other regions of the genome or to plasmid DNA present in the cells.

Because NHEJ is a mutagenic process, it may also be used to delete small sequence motifs as long as the generation of a specific final sequence is not required. If a double-strand break is targeted near to a short target sequence, the deletion mutations caused by the NHEJ repair often span, and therefore remove, the unwanted nucleotides. For the deletion of larger DNA segments, introducing two double-strand breaks, one on each side of the sequence, can result in NHEJ between the ends with removal of the entire intervening sequence. Both of these approaches can be used to delete specific DNA sequences; however, the error-prone nature of NHEJ may still produce indel mutations at the site of repair.

Both double strand cleaving by the CRISPR/Cas system can be used in the methods and compositions described herein to generate NHEJ-mediated indels. NHEJ-mediated indels targeted to the gene, e.g., a coding region, e.g., an early coding region of a gene of interest can be used to knockout (i.e., eliminate expression of) a gene of interest. For example, early coding region of a gene of interest includes sequence immediately following a transcription start site, within a first exon of the coding sequence, or within 500 bp of the transcription start site (e.g., less than 500, 450, 400, 350, 300, 250, 200, 150, 100 or 50 bp).

In an embodiment, in which the CRISPR/Cas system generates a double strand break for the purpose of inducing NHEJ-mediated indels, a guide RNA may be configured to position one double-strand break in close proximity to a nucleotide of the target position. In an embodiment, the cleavage site may be between 0-500 bp away from the target position (e.g., less than 500, 400, 300, 200, 100, 50, 40, 30, 25, 20, 15, 10, 9, 8, 7, 6, 5, 4, 3, 2 or 1 bp from the target position).

In an embodiment, in which two guide RNAs complexing with CRISPR/Cas system nickases induce two single strand breaks for the purpose of inducing NHEJ-mediated indels, two guide RNAs may be configured to position two single-strand breaks to provide for NHEJ repair a nucleotide of the target position.

dCas and Functional Effectors

Unlike CRISPR-Cas-mediated gene knockout, which permanently eliminates expression by mutating the gene at the DNA level, CRISPR-Cas knockdown allows for temporary reduction of gene expression through the use of artificial transcription factors. Mutating key residues in cleavage domains of the Cas protein results in the generation of a catalytically inactive Cas protein. A catalytically inactive Cas protein complexes with a guide RNA and localizes to the DNA sequence specified by that guide RNA's targeting domain, however, it does not cleave the target DNA. Fusion of the inactive Cas protein to an effector domain also referred to herein as a functional domain, e.g., a transcription repression domain, enables recruitment of the effector to any DNA site specified by the guide RNA.

In general, the positioning of the one or more functional domain on the inactivated CRISPR/Cas protein is one which allows for correct spatial orientation for the functional domain to affect the target with the attributed functional effect. For example, if the functional domain is a transcription activator (e.g., VP64 or p65), the transcription activator is placed in a spatial orientation which allows it to affect the transcription of the target. Likewise, a transcription repressor will be advantageously positioned to affect the transcription of the target, and a nuclease (e.g., FokI) will be advantageously positioned to cleave or partially cleave the target. This may include positions other than the N-/C-terminus of the CRISPR protein.

In certain embodiments, Cas protein may be fused to a transcriptional repression domain and recruited to the promoter region of a gene. Especially for gene repression, it is contemplated herein that blocking the binding site of an endogenous transcription factor would aid in downregulating gene expression.

In an embodiment, a guide RNA molecule can be targeted to a known transcription response elements (e.g., promoters, enhancers, etc.), a known upstream activating sequences, and/or sequences of unknown or known function that are suspected of being able to control expression of the target DNA. Idem: adapt to refer to regions with the motifs of interest

In some methods, a target polynucleotide can be inactivated to affect the modification of the expression in a cell. For example, upon the binding of a CRISPR complex to a target sequence in a cell, the target polynucleotide is inactivated such that the sequence is not transcribed, the coded protein is not produced, or the sequence does not function as the wild-type sequence does. For example, a protein or microRNA coding sequence may be inactivated such that the protein is not produced.

### Base Editing

The genetic modifying agents may be one or more components of a base editing system. In general, a base editor comprises a Cas protein or a variant thereof (e.g., an inactive or nuclease form of Cas protein) fused with a deaminase or a variant thereof. In some embodiments, compositions herein comprise nucleotide sequence comprising encoding sequences for one or more components of a base editing system. A base-editing system may comprise a deaminase (e.g., an adenosine deaminase or cytidine deaminase) fused with a Cas protein. The Cas protein may be a dead Cas protein or a Cas nickase protein. In certain examples, the system comprises a mutated form of an adenosine deaminase fused with a dead CRISPR-Cas or CRISPR-Cas nickase. The mutated form of the adenosine deaminase may have both adenosine deaminase and cytidine deaminase activities. In certain example embodiments, a dCas13b can be fused with an adenosine deaminase or cytidine deaminase for base editing purposes. In some cases, the dCas13b is dCas13b-t1, dCas13b-t2, or dCas13b-t3.

For example, the CRISPR-Cas system may comprise a dead Cas (dCas) fused or otherwise linked to a nucleotide deaminase. The nucleotide deaminase may be capable of nucleic acid editing, e.g., DNA editing or RNA editing. In certain examples, the nucleotide deaminase is capable of altering mRNA splicing by editing mRNA. In some cases, the nucleotide deaminase may be a cytidine deaminase. In certain cases, the nucleotide deaminase may be an adenosine deaminase. The dead Cas protein may be dCas9, dCas12, or dCas13. The nucleotide sequences may comprise encoding sequences for the nucleotide deaminase. The nucleotide sequences may comprise coding sequences for the dead Cas proteins.

In one aspect, the present disclosure provides an engineered adenosine deaminase. The engineered adenosine deaminase may comprise one or more mutations herein. In some embodiments, the engineered adenosine deaminase has cytidine deaminase activity. In certain examples, the engineered adenosine deaminase has both cytidine deaminase activity and adenosine deaminase.

### Adenosine Deaminase

The term “adenosine deaminase” or “adenosine deaminase protein” as used herein refers to a protein, a polypeptide, or one or more functional domain(s) of a protein or a polypeptide that is capable of catalyzing a hydrolytic deamination reaction that converts an adenine (or an adenine moiety of a molecule) to a hypoxanthine (or a hypoxanthine moiety of a molecule), as shown below. In some embodiments, the adenine-containing molecule is an adenosine (A), and the hypoxanthine-containing molecule is an inosine (I). The adenine-containing molecule can be deoxyribonucleic acid (DNA) or ribonucleic acid (RNA).

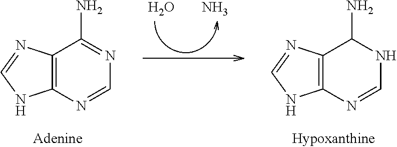

According to the present disclosure, adenosine deaminases that can be used in connection with the present disclosure include, but are not limited to, members of the enzyme family known as adenosine deaminases that act on RNA (ADARs), members of the enzyme family known as adenosine deaminases that act on tRNA (ADATs), and other adenosine deaminase domain-containing (ADAD) family members. According to the present disclosure, the adenosine deaminase is capable of targeting adenine in a RNA/DNA and RNA duplexes. Indeed, Zheng et al. (Nucleic Acids Res. 2017, 45(6): 3369-3377) demonstrate that ADARs can carry out adenosine to inosine editing reactions on RNA/DNA and RNA/RNA duplexes. In particular embodiments, the adenosine deaminase has been modified to increase its ability to edit DNA in a RNA/DNA heteroduplex of in an RNA duplex as detailed herein below.

In some embodiments, the adenosine deaminase is derived from one or more metazoa species, including but not limited to, mammals, birds, frogs, squids, fish, flies and worms. In some embodiments, the adenosine deaminase is a human, squid or Drosophila adenosine deaminase.

In some embodiments, the adenosine deaminase is a human ADAR, including hADAR1, hADAR2, hADAR3. In some embodiments, the adenosine deaminase is a Caenorhabditis elegans ADAR protein, including ADR-1 and ADR-2. In some embodiments, the adenosine deaminase is a Drosophila ADAR protein, including dAdar. In some embodiments, the adenosine deaminase is a squid Loligo pealeii ADAR protein, including sqADAR2a and sqADAR2b. In some embodiments, the adenosine deaminase is a human ADAT protein. In some embodiments, the adenosine deaminase is a Drosophila ADAT protein. In some embodiments, the adenosine deaminase is a human ADAD protein, including TENR (hADAD1) and TENRL (hADAD2).

In some embodiments, the adenosine deaminase is a TadA protein such as E. coli TadA. See Kim et al., Biochemistry 45:6407-6416 (2006); Wolf et al., EMBO J. 21:3841-3851 (2002). In some embodiments, the adenosine deaminase is mouse ADA. See Grunebaum et al., Curr. Opin. Allergy Clin. Immunol. 13:630-638 (2013). In some embodiments, the adenosine deaminase is human ADAT2. See Fukui et al., J. Nucleic Acids 2010:260512 (2010). In some embodiments, the deaminase (e.g., adenosine or cytidine deaminase) is one or more of those described in Cox et al., Science. 2017, Nov. 24; 358(6366): 1019-1027; Komore et al., Nature. 2016 May 19; 533(7603):420-4; and Gaudelli et al., Nature. 2017 Nov. 23; 551(7681):464-471.

In some embodiments, the adenosine deaminase protein recognizes and converts one or more target adenosine residue(s) in a double-stranded nucleic acid substrate into inosine residues (s). In some embodiments, the double-stranded nucleic acid substrate is a RNA-DNA hybrid duplex. In some embodiments, the adenosine deaminase protein recognizes a binding window on the double-stranded substrate. In some embodiments, the binding window contains at least one target adenosine residue(s). In some embodiments, the binding window is in the range of about 3 bp to about 100 bp. In some embodiments, the binding window is in the range of about 5 bp to about 50 bp. In some embodiments, the binding window is in the range of about 10 bp to about 30 bp. In some embodiments, the binding window is about 1 bp, 2 bp, 3 bp, 5 bp, 7 bp, 10 bp, 15 bp, 20 bp, 25 bp, 30 bp, 40 bp, 45 bp, 50 bp, 55 bp, 60 bp, 65 bp, 70 bp, 75 bp, 80 bp, 85 bp, 90 bp, 95 bp, or 100 bp.

In some embodiments, the adenosine deaminase protein comprises one or more deaminase domains. Not intended to be bound by a particular theory, it is contemplated that the deaminase domain functions to recognize and convert one or more target adenosine (A) residue(s) contained in a double-stranded nucleic acid substrate into inosine (I) residue(s). In some embodiments, the deaminase domain comprises an active center. In some embodiments, the active center comprises a zinc ion. In some embodiments, during the A-to-I editing process, base pairing at the target adenosine residue is disrupted, and the target adenosine residue is “flipped” out of the double helix to become accessible by the adenosine deaminase. In some embodiments, amino acid residues in or near the active center interact with one or more nucleotide(s) 5′ to a target adenosine residue. In some embodiments, amino acid residues in or near the active center interact with one or more nucleotide(s) 3′ to a target adenosine residue. In some embodiments, amino acid residues in or near the active center further interact with the nucleotide complementary to the target adenosine residue on the opposite strand. In some embodiments, the amino acid residues form hydrogen bonds with the 2′ hydroxyl group of the nucleotides.

In some embodiments, the adenosine deaminase comprises human ADAR2 full protein (hADAR2) or the deaminase domain thereof (hADAR2-D). In some embodiments, the adenosine deaminase is an ADAR family member that is homologous to hADAR2 or hADAR2-D.

Particularly, in some embodiments, the homologous ADAR protein is human ADAR1 (hADAR1) or the deaminase domain thereof (hADAR1-D). In some embodiments, glycine 1007 of hADAR1-D corresponds to glycine 487 hADAR2-D, and glutamic Acid 1008 of hADAR1-D corresponds to glutamic acid 488 of hADAR2-D.

In some embodiments, the adenosine deaminase comprises the wild-type amino acid sequence of hADAR2-D. In some embodiments, the adenosine deaminase comprises one or more mutations in the hADAR2-D sequence, such that the editing efficiency, and/or substrate editing preference of hADAR2-D is changed according to specific needs. The engineered adenosine deaminase may be fused with a Cas protein, e.g., Cas9, Cas 12 (e.g., Cas12a, Cas12b, Cas12c, Cas12d, etc.), Cas13 (e.g., Cas13a, Cas13b (such as Cas13b-t1, Cas13b-t2, Cas13b-t3), Cas13c, Cas13d, etc.), Cas14, CasX, CasY, or an engineered form of the Cas protein (e.g., an invective, dead form, a nickase form). In some examples, provided herein include an engineered adenosine deaminase fused with a dead Cas13b protein or Cas13 nickase.

Certain mutations of hADAR1 and hADAR2 proteins have been described in Kuttan et al., Proc Natl Acad Sci USA. (2012) 109(48):E3295-304; Want et al. ACS Chem Biol. (2015) 10(11):2512-9; and Zheng et al. Nucleic Acids Res. (2017) 45(6):3369-337, each of which is incorporated herein by reference in its entirety.

In some embodiments, the adenosine deaminase comprises a mutation at glycine336 of the hADAR2-D amino acid sequence, or a corresponding position in a homologous ADAR protein. In some embodiments, the glycine residue at position 336 is replaced by an aspartic acid residue (G336D).

In some embodiments, the adenosine deaminase comprises a mutation at Glycine487 of the hADAR2-D amino acid sequence, or a corresponding position in a homologous ADAR protein. In some embodiments, the glycine residue at position 487 is replaced by a non-polar amino acid residue with relatively small side chains. For example, in some embodiments, the glycine residue at position 487 is replaced by an alanine residue (G487A). In some embodiments, the glycine residue at position 487 is replaced by a valine residue (G487V). In some embodiments, the glycine residue at position 487 is replaced by an amino acid residue with relatively large side chains. In some embodiments, the glycine residue at position 487 is replaced by a arginine residue (G487R). In some embodiments, the glycine residue at position 487 is replaced by a lysine residue (G487K). In some embodiments, the glycine residue at position 487 is replaced by a tryptophan residue (G487W). In some embodiments, the glycine residue at position 487 is replaced by a tyrosine residue (G487Y).

In some embodiments, the adenosine deaminase comprises a mutation at glutamic acid488 of the hADAR2-D amino acid sequence, or a corresponding position in a homologous ADAR protein. In some embodiments, the glutamic acid residue at position 488 is replaced by a glutamine residue (E488Q). In some embodiments, the glutamic acid residue at position 488 is replaced by a histidine residue (E488H). In some embodiments, the glutamic acid residue at position 488 is replace by an arginine residue (E488R). In some embodiments, the glutamic acid residue at position 488 is replace by a lysine residue (E488K). In some embodiments, the glutamic acid residue at position 488 is replace by an asparagine residue (E488N). In some embodiments, the glutamic acid residue at position 488 is replace by an alanine residue (E488A). In some embodiments, the glutamic acid residue at position 488 is replace by a Methionine residue (E488M). In some embodiments, the glutamic acid residue at position 488 is replace by a serine residue (E488S). In some embodiments, the glutamic acid residue at position 488 is replace by a phenylalanine residue (E488F). In some embodiments, the glutamic acid residue at position 488 is replace by a lysine residue (E488L). In some embodiments, the glutamic acid residue at position 488 is replace by a tryptophan residue (E488W).

In some embodiments, the adenosine deaminase comprises a mutation at threonine490 of the hADAR2-D amino acid sequence, or a corresponding position in a homologous ADAR protein. In some embodiments, the threonine residue at position 490 is replaced by a cysteine residue (T490C). In some embodiments, the threonine residue at position 490 is replaced by a serine residue (T490S). In some embodiments, the threonine residue at position 490 is replaced by an alanine residue (T490A). In some embodiments, the threonine residue at position 490 is replaced by a phenylalanine residue (T490F). In some embodiments, the threonine residue at position 490 is replaced by a tyrosine residue (T490Y). In some embodiments, the threonine residue at position 490 is replaced by a serine residue (T490R). In some embodiments, the threonine residue at position 490 is replaced by an alanine residue (T490K). In some embodiments, the threonine residue at position 490 is replaced by a phenylalanine residue (T490P). In some embodiments, the threonine residue at position 490 is replaced by a tyrosine residue (T490E).

In some embodiments, the adenosine deaminase comprises a mutation at valine493 of the hADAR2-D amino acid sequence, or a corresponding position in a homologous ADAR protein. In some embodiments, the valine residue at position 493 is replaced by an alanine residue (V493A). In some embodiments, the valine residue at position 493 is replaced by a serine residue (V493S). In some embodiments, the valine residue at position 493 is replaced by a threonine residue (V493T). In some embodiments, the valine residue at position 493 is replaced by an arginine residue (V493R). In some embodiments, the valine residue at position 493 is replaced by an aspartic acid residue (V493D). In some embodiments, the valine residue at position 493 is replaced by a proline residue (V493P). In some embodiments, the valine residue at position 493 is replaced by a glycine residue (V493G).

In some embodiments, the adenosine deaminase comprises a mutation at alanine589 of the hADAR2-D amino acid sequence, or a corresponding position in a homologous ADAR protein. In some embodiments, the alanine residue at position 589 is replaced by a valine residue (A589V).

In some embodiments, the adenosine deaminase comprises a mutation at asparagine597 of the hADAR2-D amino acid sequence, or a corresponding position in a homologous ADAR protein. In some embodiments, the asparagine residue at position 597 is replaced by a lysine residue (N597K). In some embodiments, the adenosine deaminase comprises a mutation at position 597 of the amino acid sequence, which has an asparagine residue in the wild type sequence. In some embodiments, the asparagine residue at position 597 is replaced by an arginine residue (N597R). In some embodiments, the adenosine deaminase comprises a mutation at position 597 of the amino acid sequence, which has an asparagine residue in the wild type sequence. In some embodiments, the asparagine residue at position 597 is replaced by an alanine residue (N597A). In some embodiments, the adenosine deaminase comprises a mutation at position 597 of the amino acid sequence, which has an asparagine residue in the wild type sequence. In some embodiments, the asparagine residue at position 597 is replaced by a glutamic acid residue (N597E). In some embodiments, the adenosine deaminase comprises a mutation at position 597 of the amino acid sequence, which has an asparagine residue in the wild type sequence. In some embodiments, the asparagine residue at position 597 is replaced by a histidine residue (N597H). In some embodiments, the adenosine deaminase comprises a mutation at position 597 of the amino acid sequence, which has an asparagine residue in the wild type sequence. In some embodiments, the asparagine residue at position 597 is replaced by a glycine residue (N597G). In some embodiments, the adenosine deaminase comprises a mutation at position 597 of the amino acid sequence, which has an asparagine residue in the wild type sequence. In some embodiments, the asparagine residue at position 597 is replaced by a tyrosine residue (N597Y). In some embodiments, the asparagine residue at position 597 is replaced by a phenylalanine residue (N597F). In some embodiments, the adenosine deaminase comprises mutation N597I. In some embodiments, the adenosine deaminase comprises mutation N597L. In some embodiments, the adenosine deaminase comprises mutation N597V. In some embodiments, the adenosine deaminase comprises mutation N597M. In some embodiments, the adenosine deaminase comprises mutation N597C. In some embodiments, the adenosine deaminase comprises mutation N597P. In some embodiments, the adenosine deaminase comprises mutation N597T. In some embodiments, the adenosine deaminase comprises mutation N597S. In some embodiments, the adenosine deaminase comprises mutation N597W. In some embodiments, the adenosine deaminase comprises mutation N597Q. In some embodiments, the adenosine deaminase comprises mutation N597D. In certain example embodiments, the mutations at N597 described above are further made in the context of an E488Q background

In some embodiments, the adenosine deaminase comprises a mutation at serine599 of the hADAR2-D amino acid sequence, or a corresponding position in a homologous ADAR protein. In some embodiments, the serine residue at position 599 is replaced by a threonine residue (S599T).

In some embodiments, the adenosine deaminase comprises a mutation at asparagine613 of the hADAR2-D amino acid sequence, or a corresponding position in a homologous ADAR protein. In some embodiments, the asparagine residue at position 613 is replaced by a lysine residue (N613K). In some embodiments, the adenosine deaminase comprises a mutation at position 613 of the amino acid sequence, which has an asparagine residue in the wild type sequence. In some embodiments, the asparagine residue at position 613 is replaced by an arginine residue (N613R). In some embodiments, the adenosine deaminase comprises a mutation at position 613 of the amino acid sequence, which has an asparagine residue in the wild type sequence. In some embodiments, the asparagine residue at position 613 is replaced by an alanine residue (N613A) In some embodiments, the adenosine deaminase comprises a mutation at position 613 of the amino acid sequence, which has an asparagine residue in the wild type sequence. In some embodiments, the asparagine residue at position 613 is replaced by a glutamic acid residue (N613E). In some embodiments, the adenosine deaminase comprises mutation N613I. In some embodiments, the adenosine deaminase comprises mutation N613L. In some embodiments, the adenosine deaminase comprises mutation N613V. In some embodiments, the adenosine deaminase comprises mutation N613F. In some embodiments, the adenosine deaminase comprises mutation N613M. In some embodiments, the adenosine deaminase comprises mutation N613C. In some embodiments, the adenosine deaminase comprises mutation N613G. In some embodiments, the adenosine deaminase comprises mutation N613P. In some embodiments, the adenosine deaminase comprises mutation N613T. In some embodiments, the adenosine deaminase comprises mutation N613S. In some embodiments, the adenosine deaminase comprises mutation N613Y. In some embodiments, the adenosine deaminase comprises mutation N613W. In some embodiments, the adenosine deaminase comprises mutation N613Q. In some embodiments, the adenosine deaminase comprises mutation N613H. In some embodiments, the adenosine deaminase comprises mutation N613D. In some embodiments, the mutations at N613 described above are further made in combination with a E488Q mutation.

In some embodiments, to improve editing efficiency, the adenosine deaminase may comprise one or more of the mutations: G336D, G487A, G487V, E488Q, E488H, E488R, E488N, E488A, E488S, E488M, T490C, T490S, V493T, V493S, V493A, V493R, V493D, V493P, V493G, N597K, N597R, N597A, N597E, N597H, N597G, N597Y, A589V, S599T, N613K, N613R, N613A, N613E, based on amino acid sequence positions of hADAR2-D, and mutations in a homologous ADAR protein corresponding to the above.

In some embodiments, to reduce editing efficiency, the adenosine deaminase may comprise one or more of the mutations: E488F, E488L, E488W, T490A, T490F, T490Y, T490R, T490K, T490P, T490E, N597F, based on amino acid sequence positions of hADAR2-D, and mutations in a homologous ADAR protein corresponding to the above. In particular embodiments, it can be of interest to use an adenosine deaminase enzyme with reduced efficacy to reduce off-target effects.

In some embodiments, to reduce off-target effects, the adenosine deaminase comprises one or more of mutations at R348, V351, T375, K376, E396, C451, R455, N473, R474, K475, R477, R481, S486, E488, T490, S495, R510, based on amino acid sequence positions of hADAR2-D, and mutations in a homologous ADAR protein corresponding to the above. In some embodiments, the adenosine deaminase comprises mutation at E488 and one or more additional positions selected from R348, V351, T375, K376, E396, C451, R455, N473, R474, K475, R477, R481, S486, T490, S495, R510. In some embodiments, the adenosine deaminase comprises mutation at T375, and optionally at one or more additional positions. In some embodiments, the adenosine deaminase comprises mutation at N473, and optionally at one or more additional positions. In some embodiments, the adenosine deaminase comprises mutation at V351, and optionally at one or more additional positions. In some embodiments, the adenosine deaminase comprises mutation at E488 and T375, and optionally at one or more additional positions. In some embodiments, the adenosine deaminase comprises mutation at E488 and N473, and optionally at one or more additional positions. In some embodiments, the adenosine deaminase comprises mutation E488 and V351, and optionally at one or more additional positions. In some embodiments, the adenosine deaminase comprises mutation at E488 and one or more of T375, N473, and V351.

In some embodiments, to reduce off-target effects, the adenosine deaminase comprises one or more of mutations selected from R348E, V351L, T375G, T375S, R455G, R455S, R455E, N473D, R474E, K475Q, R477E, R481E, S486T, E488Q, T490A, T490S, S495T, and R510E, based on amino acid sequence positions of hADAR2-D, and mutations in a homologous ADAR protein corresponding to the above. In some embodiments, the adenosine deaminase comprises mutation E488Q and one or more additional mutations selected from R348E, V351L, T375G, T375S, R455G, R455S, R455E, N473D, R474E, K475Q, R477E, R481E, S486T, T490A, T490S, S495T, and R510E. In some embodiments, the adenosine deaminase comprises mutation T375G or T375S, and optionally one or more additional mutations. In some embodiments, the adenosine deaminase comprises mutation N473D, and optionally one or more additional mutations. In some embodiments, the adenosine deaminase comprises mutation V351L, and optionally one or more additional mutations. In some embodiments, the adenosine deaminase comprises mutation E488Q, and T375G or T375G, and optionally one or more additional mutations. In some embodiments, the adenosine deaminase comprises mutation E488Q and N473D, and optionally one or more additional mutations. In some embodiments, the adenosine deaminase comprises mutation E488Q and V351L, and optionally one or more additional mutations. In some embodiments, the adenosine deaminase comprises mutation E488Q and one or more of T375G/S, N473D and V351L.

In certain examples, the adenosine deaminase protein or catalytic domain thereof has been modified to comprise a mutation at E488, preferably E488Q, of the hADAR2-D amino acid sequence, or a corresponding position in a homologous ADAR protein and/or wherein the adenosine deaminase protein or catalytic domain thereof has been modified to comprise a mutation at T375, preferably T375G of the hADAR2-D amino acid sequence, or a corresponding position in a homologous ADAR protein. In certain examples, the adenosine deaminase protein or catalytic domain thereof has been modified to comprise a mutation at E1008, preferably E1008Q, of the hADAR1d amino acid sequence, or a corresponding position in a homologous ADAR protein.

Crystal structures of the human ADAR2 deaminase domain bound to duplex RNA reveal a protein loop that binds the RNA on the 5′ side of the modification site. This 5′ binding loop is one contributor to substrate specificity differences between ADAR family members. See Wang et al., Nucleic Acids Res., 44(20):9872-9880 (2016), the content of which is incorporated herein by reference in its entirety. In addition, an ADAR2-specific RNA-binding loop was identified near the enzyme active site. See Mathews et al., Nat. Struct. Mol. Biol., 23(5):426-33 (2016), the content of which is incorporated herein by reference in its entirety. In some embodiments, the adenosine deaminase comprises one or more mutations in the RNA binding loop to improve editing specificity and/or efficiency.

In some embodiments, the adenosine deaminase comprises a mutation at alanine454 of the hADAR2-D amino acid sequence, or a corresponding position in a homologous ADAR protein. In some embodiments, the alanine residue at position 454 is replaced by a serine residue (A454S). In some embodiments, the alanine residue at position 454 is replaced by a cysteine residue (A454C). In some embodiments, the alanine residue at position 454 is replaced by an aspartic acid residue (A454D).

In some embodiments, the adenosine deaminase comprises a mutation at arginine455 of the hADAR2-D amino acid sequence, or a corresponding position in a homologous ADAR protein. In some embodiments, the arginine residue at position 455 is replaced by an alanine residue (R455A). In some embodiments, the arginine residue at position 455 is replaced by a valine residue (R455V). In some embodiments, the arginine residue at position 455 is replaced by a histidine residue (R455H). In some embodiments, the arginine residue at position 455 is replaced by a glycine residue (R455G). In some embodiments, the arginine residue at position 455 is replaced by a serine residue (R455S). In some embodiments, the arginine residue at position 455 is replaced by a glutamic acid residue (R455E). In some embodiments, the adenosine deaminase comprises mutation R455C. In some embodiments, the adenosine deaminase comprises mutation R455I. In some embodiments, the adenosine deaminase comprises mutation R455K. In some embodiments, the adenosine deaminase comprises mutation R455L. In some embodiments, the adenosine deaminase comprises mutation R455M. In some embodiments, the adenosine deaminase comprises mutation R455N. In some embodiments, the adenosine deaminase comprises mutation R455Q. In some embodiments, the adenosine deaminase comprises mutation R455F. In some embodiments, the adenosine deaminase comprises mutation R455W. In some embodiments, the adenosine deaminase comprises mutation R455P. In some embodiments, the adenosine deaminase comprises mutation R455Y. In some embodiments, the adenosine deaminase comprises mutation R455E. In some embodiments, the adenosine deaminase comprises mutation R455D. In some embodiments, the mutations at R455 described above are further made in combination with a E488Q mutation.

In some embodiments, the adenosine deaminase comprises a mutation at isoleucine456 of the hADAR2-D amino acid sequence, or a corresponding position in a homologous ADAR protein. In some embodiments, the isoleucine residue at position 456 is replaced by a valine residue (I456V). In some embodiments, the isoleucine residue at position 456 is replaced by a leucine residue (I456L). In some embodiments, the isoleucine residue at position 456 is replaced by an aspartic acid residue (I456D).

In some embodiments, the adenosine deaminase comprises a mutation at phenylalanine457 of the hADAR2-D amino acid sequence, or a corresponding position in a homologous ADAR protein. In some embodiments, the phenylalanine residue at position 457 is replaced by a tyrosine residue (F457Y). In some embodiments, the phenylalanine residue at position 457 is replaced by an arginine residue (F457R). In some embodiments, the phenylalanine residue at position 457 is replaced by a glutamic acid residue (F457E).

In some embodiments, the adenosine deaminase comprises a mutation at serine458 of the hADAR2-D amino acid sequence, or a corresponding position in a homologous ADAR protein. In some embodiments, the serine residue at position 458 is replaced by a valine residue (S458V). In some embodiments, the serine residue at position 458 is replaced by a phenylalanine residue (S458F). In some embodiments, the serine residue at position 458 is replaced by a proline residue (S458P). In some embodiments, the adenosine deaminase comprises mutation S458I. In some embodiments, the adenosine deaminase comprises mutation S458L. In some embodiments, the adenosine deaminase comprises mutation S458M. In some embodiments, the adenosine deaminase comprises mutation S458C. In some embodiments, the adenosine deaminase comprises mutation S458A. In some embodiments, the adenosine deaminase comprises mutation S458G. In some embodiments, the adenosine deaminase comprises mutation S458T. In some embodiments, the adenosine deaminase comprises mutation S458Y. In some embodiments, the adenosine deaminase comprises mutation S458W. In some embodiments, the adenosine deaminase comprises mutation S458Q. In some embodiments, the adenosine deaminase comprises mutation S458N. In some embodiments, the adenosine deaminase comprises mutation S458H. In some embodiments, the adenosine deaminase comprises mutation S458E. In some embodiments, the adenosine deaminase comprises mutation S458D. In some embodiments, the adenosine deaminase comprises mutation S458K. In some embodiments, the adenosine deaminase comprises mutation S458R. In some embodiments, the mutations at S458 described above are further made in combination with a E488Q mutation.

In some embodiments, the adenosine deaminase comprises a mutation at proline459 of the hADAR2-D amino acid sequence, or a corresponding position in a homologous ADAR protein. In some embodiments, the proline residue at position 459 is replaced by a cysteine residue (P459C). In some embodiments, the proline residue at position 459 is replaced by a histidine residue (P459H). In some embodiments, the proline residue at position 459 is replaced by a tryptophan residue (P459W).

In some embodiments, the adenosine deaminase comprises a mutation at histidine460 of the hADAR2-D amino acid sequence, or a corresponding position in a homologous ADAR protein. In some embodiments, the histidine residue at position 460 is replaced by an arginine residue (H460R). In some embodiments, the histidine residue at position 460 is replaced by an isoleucine residue (H460I). In some embodiments, the histidine residue at position 460 is replaced by a proline residue (H460P). In some embodiments, the adenosine deaminase comprises mutation H460L. In some embodiments, the adenosine deaminase comprises mutation H460V. In some embodiments, the adenosine deaminase comprises mutation H460F. In some embodiments, the adenosine deaminase comprises mutation H460M. In some embodiments, the adenosine deaminase comprises mutation H460C. In some embodiments, the adenosine deaminase comprises mutation H460A. In some embodiments, the adenosine deaminase comprises mutation H460G. In some embodiments, the adenosine deaminase comprises mutation H460T. In some embodiments, the adenosine deaminase comprises mutation H460S. In some embodiments, the adenosine deaminase comprises mutation H460Y. In some embodiments, the adenosine deaminase comprises mutation H460W. In some embodiments, the adenosine deaminase comprises mutation H460Q. In some embodiments, the adenosine deaminase comprises mutation H460N. In some embodiments, the adenosine deaminase comprises mutation H460E. In some embodiments, the adenosine deaminase comprises mutation H460D. In some embodiments, the adenosine deaminase comprises mutation H460K. In some embodiments, the mutations at H460 described above are further made in combination with a E488Q mutation.

In some embodiments, the adenosine deaminase comprises a mutation at proline462 of the hADAR2-D amino acid sequence, or a corresponding position in a homologous ADAR protein. In some embodiments, the proline residue at position 462 is replaced by a serine residue (P462S). In some embodiments, the proline residue at position 462 is replaced by a tryptophan residue (P462W). In some embodiments, the proline residue at position 462 is replaced by a glutamic acid residue (P462E).

In some embodiments, the adenosine deaminase comprises a mutation at aspartic acid469 of the hADAR2-D amino acid sequence, or a corresponding position in a homologous ADAR protein. In some embodiments, the aspartic acid residue at position 469 is replaced by a glutamine residue (D469Q). In some embodiments, the aspartic acid residue at position 469 is replaced by a serine residue (D469S). In some embodiments, the aspartic acid residue at position 469 is replaced by a tyrosine residue (D469Y).

In some embodiments, the adenosine deaminase comprises a mutation at arginine470 of the hADAR2-D amino acid sequence, or a corresponding position in a homologous ADAR protein. In some embodiments, the arginine residue at position 470 is replaced by an alanine residue (R470A). In some embodiments, the arginine residue at position 470 is replaced by an isoleucine residue (R470I). In some embodiments, the arginine residue at position 470 is replaced by an aspartic acid residue (R470D).

In some embodiments, the adenosine deaminase comprises a mutation at histidine471 of the hADAR2-D amino acid sequence, or a corresponding position in a homologous ADAR protein. In some embodiments, the histidine residue at position 471 is replaced by a lysine residue (H471K). In some embodiments, the histidine residue at position 471 is replaced by a threonine residue (H471T). In some embodiments, the histidine residue at position 471 is replaced by a valine residue (H471V).

In some embodiments, the adenosine deaminase comprises a mutation at proline472 of the hADAR2-D amino acid sequence, or a corresponding position in a homologous ADAR protein. In some embodiments, the proline residue at position 472 is replaced by a lysine residue (P472K). In some embodiments, the proline residue at position 472 is replaced by a threonine residue (P472T). In some embodiments, the proline residue at position 472 is replaced by an aspartic acid residue (P472D).

In some embodiments, the adenosine deaminase comprises a mutation at asparagine473 of the hADAR2-D amino acid sequence, or a corresponding position in a homologous ADAR protein. In some embodiments, the asparagine residue at position 473 is replaced by an arginine residue (N473R). In some embodiments, the asparagine residue at position 473 is replaced by a tryptophan residue (N473W). In some embodiments, the asparagine residue at position 473 is replaced by a proline residue (N473P). In some embodiments, the asparagine residue at position 473 is replaced by an aspartic acid residue (N473D).

In some embodiments, the adenosine deaminase comprises a mutation at arginine 474 of the hADAR2-D amino acid sequence, or a corresponding position in a homologous ADAR protein. In some embodiments, the arginine residue at position 474 is replaced by a lysine residue (R474K). In some embodiments, the arginine residue at position 474 is replaced by a glycine residue (R474G). In some embodiments, the arginine residue at position 474 is replaced by an aspartic acid residue (R474D). In some embodiments, the arginine residue at position 474 is replaced by a glutamic acid residue (R474E).

In some embodiments, the adenosine deaminase comprises a mutation at lysine475 of the hADAR2-D amino acid sequence, or a corresponding position in a homologous ADAR protein. In some embodiments, the lysine residue at position 475 is replaced by a glutamine residue (K475Q). In some embodiments, the lysine residue at position 475 is replaced by an asparagine residue (K475N). In some embodiments, the lysine residue at position 475 is replaced by an aspartic acid residue (K475D).

In some embodiments, the adenosine deaminase comprises a mutation at alanine476 of the hADAR2-D amino acid sequence, or a corresponding position in a homologous ADAR protein. In some embodiments, the alanine residue at position 476 is replaced by a serine residue (A476S). In some embodiments, the alanine residue at position 476 is replaced by an arginine residue (A476R). In some embodiments, the alanine residue at position 476 is replaced by a glutamic acid residue (A476E).

In some embodiments, the adenosine deaminase comprises a mutation at arginine477 of the hADAR2-D amino acid sequence, or a corresponding position in a homologous ADAR protein. In some embodiments, the arginine residue at position 477 is replaced by a lysine residue (R477K). In some embodiments, the arginine residue at position 477 is replaced by a threonine residue (R477T). In some embodiments, the arginine residue at position 477 is replaced by a phenylalanine residue (R477F). In some embodiments, the arginine residue at position 474 is replaced by a glutamic acid residue (R477E).

In some embodiments, the adenosine deaminase comprises a mutation at glycine478 of the hADAR2-D amino acid sequence, or a corresponding position in a homologous ADAR protein. In some embodiments, the glycine residue at position 478 is replaced by an alanine residue (G478A). In some embodiments, the glycine residue at position 478 is replaced by an arginine residue (G478R). In some embodiments, the glycine residue at position 478 is replaced by a tyrosine residue (G478Y). In some embodiments, the adenosine deaminase comprises mutation G478I. In some embodiments, the adenosine deaminase comprises mutation G478L. In some embodiments, the adenosine deaminase comprises mutation G478V. In some embodiments, the adenosine deaminase comprises mutation G478F. In some embodiments, the adenosine deaminase comprises mutation G478M. In some embodiments, the adenosine deaminase comprises mutation G478C. In some embodiments, the adenosine deaminase comprises mutation G478P. In some embodiments, the adenosine deaminase comprises mutation G478T. In some embodiments, the adenosine deaminase comprises mutation G478S. In some embodiments, the adenosine deaminase comprises mutation G478W. In some embodiments, the adenosine deaminase comprises mutation G478Q. In some embodiments, the adenosine deaminase comprises mutation G478N. In some embodiments, the adenosine deaminase comprises mutation G478H. In some embodiments, the adenosine deaminase comprises mutation G478E. In some embodiments, the adenosine deaminase comprises mutation G478D. In some embodiments, the adenosine deaminase comprises mutation G478K. In some embodiments, the mutations at G478 described above are further made in combination with a E488Q mutation.

In some embodiments, the adenosine deaminase comprises a mutation at glutamine479 of the hADAR2-D amino acid sequence, or a corresponding position in a homologous ADAR protein. In some embodiments, the glutamine residue at position 479 is replaced by an asparagine residue (Q479N). In some embodiments, the glutamine residue at position 479 is replaced by a serine residue (Q479S). In some embodiments, the glutamine residue at position 479 is replaced by a proline residue (Q479P).

In some embodiments, the adenosine deaminase comprises a mutation at arginine348 of the hADAR2-D amino acid sequence, or a corresponding position in a homologous ADAR protein. In some embodiments, the arginine residue at position 348 is replaced by an alanine residue (R348A). In some embodiments, the arginine residue at position 348 is replaced by a glutamic acid residue (R348E).

In some embodiments, the adenosine deaminase comprises a mutation at valine351 of the hADAR2-D amino acid sequence, or a corresponding position in a homologous ADAR protein. In some embodiments, the valine residue at position 351 is replaced by a leucine residue (V351L). In some embodiments, the adenosine deaminase comprises mutation V351Y. In some embodiments, the adenosine deaminase comprises mutation V351M. In some embodiments, the adenosine deaminase comprises mutation V351T. In some embodiments, the adenosine deaminase comprises mutation V351G. In some embodiments, the adenosine deaminase comprises mutation V351A. In some embodiments, the adenosine deaminase comprises mutation V351F. In some embodiments, the adenosine deaminase comprises mutation V351E. In some embodiments, the adenosine deaminase comprises mutation V351I. In some embodiments, the adenosine deaminase comprises mutation V351C. In some embodiments, the adenosine deaminase comprises mutation V351H. In some embodiments, the adenosine deaminase comprises mutation V351P. In some embodiments, the adenosine deaminase comprises mutation V351S. In some embodiments, the adenosine deaminase comprises mutation V351K. In some embodiments, the adenosine deaminase comprises mutation V351N. In some embodiments, the adenosine deaminase comprises mutation V351W. In some embodiments, the adenosine deaminase comprises mutation V351Q. In some embodiments, the adenosine deaminase comprises mutation V351D. In some embodiments, the adenosine deaminase comprises mutation V351R. In some embodiments, the mutations at V351 described above are further made in combination with a E488Q mutation.

In some embodiments, the adenosine deaminase comprises a mutation at threonine375 of the hADAR2-D amino acid sequence, or a corresponding position in a homologous ADAR protein. In some embodiments, the threonine residue at position 375 is replaced by a glycine residue (T375G). In some embodiments, the threonine residue at position 375 is replaced by a serine residue (T375S). In some embodiments, the adenosine deaminase comprises mutation T375H. In some embodiments, the adenosine deaminase comprises mutation T375Q. In some embodiments, the adenosine deaminase comprises mutation T375C. In some embodiments, the adenosine deaminase comprises mutation T375N. In some embodiments, the adenosine deaminase comprises mutation T375M. In some embodiments, the adenosine deaminase comprises mutation T375A. In some embodiments, the adenosine deaminase comprises mutation T375W. In some embodiments, the adenosine deaminase comprises mutation T375V. In some embodiments, the adenosine deaminase comprises mutation T375R. In some embodiments, the adenosine deaminase comprises mutation T375E. In some embodiments, the adenosine deaminase comprises mutation T375K. In some embodiments, the adenosine deaminase comprises mutation T375F. In some embodiments, the adenosine deaminase comprises mutation T375I. In some embodiments, the adenosine deaminase comprises mutation T375D. In some embodiments, the adenosine deaminase comprises mutation T375P. In some embodiments, the adenosine deaminase comprises mutation T375L. In some embodiments, the adenosine deaminase comprises mutation T375Y. In some embodiments, the mutations at T375Y described above are further made in combination with an E488Q mutation.

In some embodiments, the adenosine deaminase comprises a mutation at Arg481 of the hADAR2-D amino acid sequence, or a corresponding position in a homologous ADAR protein. In some embodiments, the arginine residue at position 481 is replaced by a glutamic acid residue (R481E).

In some embodiments, the adenosine deaminase comprises a mutation at Ser486 of the hADAR2-D amino acid sequence, or a corresponding position in a homologous ADAR protein. In some embodiments, the serine residue at position 486 is replaced by a threonine residue (S486T).

In some embodiments, the adenosine deaminase comprises a mutation at Thr490 of the hADAR2-D amino acid sequence, or a corresponding position in a homologous ADAR protein. In some embodiments, the threonine residue at position 490 is replaced by an alanine residue (T490A). In some embodiments, the threonine residue at position 490 is replaced by a serine residue (T490S).

In some embodiments, the adenosine deaminase comprises a mutation at Ser495 of the hADAR2-D amino acid sequence, or a corresponding position in a homologous ADAR protein. In some embodiments, the serine residue at position 495 is replaced by a threonine residue (S495T).

In some embodiments, the adenosine deaminase comprises a mutation at Arg510 of the hADAR2-D amino acid sequence, or a corresponding position in a homologous ADAR protein. In some embodiments, the arginine residue at position 510 is replaced by a glutamine residue (R510Q). In some embodiments, the arginine residue at position 510 is replaced by an alanine residue (R510A). In some embodiments, the arginine residue at position 510 is replaced by a glutamic acid residue (R510E).

In some embodiments, the adenosine deaminase comprises a mutation at Gly593 of the hADAR2-D amino acid sequence, or a corresponding position in a homologous ADAR protein. In some embodiments, the glycine residue at position 593 is replaced by an alanine residue (G593A). In some embodiments, the glycine residue at position 593 is replaced by a glutamic acid residue (G593E).

In some embodiments, the adenosine deaminase comprises a mutation at Lys594 of the hADAR2-D amino acid sequence, or a corresponding position in a homologous ADAR protein. In some embodiments, the lysine residue at position 594 is replaced by an alanine residue (K594A).

In some embodiments, the adenosine deaminase comprises a mutation at any one or more of positions A454, R455, 1456, F457, S458, P459, H460, P462, D469, R470, H471, P472, N473, R474, K475, A476, R477, G478, Q479, R348, R510, G593, K594 of the hADAR2-D amino acid sequence, or a corresponding position in a homologous ADAR protein.

In some embodiments, the adenosine deaminase comprises any one or more of mutations A454S, A454C, A454D, R455A, R455V, R455H, I456V, I456L, I456D, F457Y, F457R, F457E, S458V, S458F, S458P, P459C, P459H, P459W, H460R, H460I, H460P, P462S, P462W, P462E, D469Q, D469S, D469Y, R470A, R470I, R470D, H471K, H471T, H471V, P472K, P472T, P472D, N473R, N473W, N473P, R474K, R474G, R474D, K475Q, K475N, K475D, A476S, A476R, A476E, R477K, R477T, R477F, G478A, G478R, G478Y, Q479N, Q479S, Q479P, R348A, R510Q, R510A, G593A, G593E, K594A of the hADAR2-D amino acid sequence, or a corresponding position in a homologous ADAR protein.

In some embodiments, the adenosine deaminase comprises a mutation at any one or more of positions T375, V351, G478, S458, H460 of the hADAR2-D amino acid sequence, or a corresponding position in a homologous ADAR protein, optionally in combination a mutation at E488. In some embodiments, the adenosine deaminase comprises one or more of mutations selected from T375G, T375C, T375H, T375Q, V351M, V351T, V351Y, G478R, S458F, H460I, optionally in combination with E488Q.

In some embodiments, the adenosine deaminase comprises one or more of mutations selected from T375H, T375Q, V351M, V351Y, H460P, optionally in combination with E488Q.

In some embodiments, the adenosine deaminase comprises mutations T375S and S458F, optionally in combination with E488Q.

In some embodiments, the adenosine deaminase comprises a mutation at two or more of positions T375, N473, R474, G478, S458, P459, V351, R455, R455, T490, R348, Q479 of the hADAR2-D amino acid sequence, or a corresponding position in a homologous ADAR protein, optionally in combination a mutation at E488. In some embodiments, the adenosine deaminase comprises two or more of mutations selected from T375G, T375S, N473D, R474E, G478R, S458F, P459W, V351L, R455G, R455S, T490A, R348E, Q479P, optionally in combination with E488Q.

In some embodiments, the adenosine deaminase comprises mutations T375G and V351L. In some embodiments, the adenosine deaminase comprises mutations T375G and R455G. In some embodiments, the adenosine deaminase comprises mutations T375G and R455S. In some embodiments, the adenosine deaminase comprises mutations T375G and T490A. In some embodiments, the adenosine deaminase comprises mutations T375G and R348E. In some embodiments, the adenosine deaminase comprises mutations T375S and V351L. In some embodiments, the adenosine deaminase comprises mutations T375S and R455G. In some embodiments, the adenosine deaminase comprises mutations T375S and R455S. In some embodiments, the adenosine deaminase comprises mutations T375S and T490A. In some embodiments, the adenosine deaminase comprises mutations T375S and R348E. In some embodiments, the adenosine deaminase comprises mutations N473D and V351L. In some embodiments, the adenosine deaminase comprises mutations N473D and R455G. In some embodiments, the adenosine deaminase comprises mutations N473D and R455S. In some embodiments, the adenosine deaminase comprises mutations N473D and T490A. In some embodiments, the adenosine deaminase comprises mutations N473D and R348E. In some embodiments, the adenosine deaminase comprises mutations R474E and V351L. In some embodiments, the adenosine deaminase comprises mutations R474E and R455G. In some embodiments, the adenosine deaminase comprises mutations R474E and R455S. In some embodiments, the adenosine deaminase comprises mutations R474E and T490A. In some embodiments, the adenosine deaminase comprises mutations R474E and R348E. In some embodiments, the adenosine deaminase comprises mutations S458F and T375G. In some embodiments, the adenosine deaminase comprises mutations S458F and T375S. In some embodiments, the adenosine deaminase comprises mutations S458F and N473D. In some embodiments, the adenosine deaminase comprises mutations S458F and R474E. In some embodiments, the adenosine deaminase comprises mutations S458F and G478R. In some embodiments, the adenosine deaminase comprises mutations G478R and T375G. In some embodiments, the adenosine deaminase comprises mutations G478R and T375S. In some embodiments, the adenosine deaminase comprises mutations G478R and N473D. In some embodiments, the adenosine deaminase comprises mutations G478R and R474E. In some embodiments, the adenosine deaminase comprises mutations P459W and T375G. In some embodiments, the adenosine deaminase comprises mutations P459W and T375S. In some embodiments, the adenosine deaminase comprises mutations P459W and N473D. In some embodiments, the adenosine deaminase comprises mutations P459W and R474E. In some embodiments, the adenosine deaminase comprises mutations P459W and G478R. In some embodiments, the adenosine deaminase comprises mutations P459W and S458F. In some embodiments, the adenosine deaminase comprises mutations Q479P and T375G. In some embodiments, the adenosine deaminase comprises mutations Q479P and T375S. In some embodiments, the adenosine deaminase comprises mutations Q479P and N473D. In some embodiments, the adenosine deaminase comprises mutations Q479P and R474E. In some embodiments, the adenosine deaminase comprises mutations Q479P and G478R. In some embodiments, the adenosine deaminase comprises mutations Q479P and S458F. In some embodiments, the adenosine deaminase comprises mutations Q479P and P459W. All mutations described in this paragraph may also further be made in combination with a E488Q mutations.

In some embodiments, the adenosine deaminase comprises a mutation at any one or more of positions K475, Q479, P459, G478, S458 of the hADAR2-D amino acid sequence, or a corresponding position in a homologous ADAR protein, optionally in combination a mutation at E488. In some embodiments, the adenosine deaminase comprises one or more of mutations selected from K475N, Q479N, P459W, G478R, S458P, S458F, optionally in combination with E488Q.

In some embodiments, the adenosine deaminase comprises a mutation at any one or more of positions T375, V351, R455, H460, A476 of the hADAR2-D amino acid sequence, or a corresponding position in a homologous ADAR protein, optionally in combination a mutation at E488. In some embodiments, the adenosine deaminase comprises one or more of mutations selected from T375G, T375C, T375H, T375Q, V351M, V351T, V351Y, R455H, H460P, H460I, A476E, optionally in combination with E488Q.

In certain embodiments, improvement of editing and reduction of off-target modification is achieved by chemical modification of gRNAs. gRNAs which are chemically modified as exemplified in Vogel et al. (2014), Angew Chem Int Ed, 53:6267-6271, doi:10.1002/anie.201402634 (incorporated herein by reference in its entirety) reduce off-target activity and improve on-target efficiency. 2′-O-methyl and phosphothioate modified guide RNAs in general improve editing efficiency in cells.

ADAR has been known to demonstrate a preference for neighboring nucleotides on either side of the edited A (www.nature.com/nsmb/journal/v23/n5/full/nsmb.3203.html, Matthews et al. (2017), Nature Structural Mol Biol, 23(5): 426-433, incorporated herein by reference in its entirety). Accordingly, in certain embodiments, the gRNA, target, and/or ADAR is selected optimized for motif preference.

Intentional mismatches have been demonstrated in vitro to allow for editing of non-preferred motifs (https://academic.oup.com/nar/article-lookup/doi/10.1093/nar/gku272; Schneider et al (2014), Nucleic Acid Res, 42(10):e87); Fukuda et al. (2017), Scienticic Reports, 7, doi:10.1038/srep41478, incorporated herein by reference in its entirety). Accordingly, in certain embodiments, to enhance RNA editing efficiency on non-preferred 5′ or 3′ neighboring bases, intentional mismatches in neighboring bases are introduced.

In some embodiments, the adenosine deaminase may be a tRNA-specific adenosine deaminase or a variant thereof. In some embodiments, the adenosine deaminase may comprise one or more of the mutations: W23L, W23R, R26G, H36L, N37S, P48S, P48T, P48A, I49V, R51L, N72D, L84F, S97C, A106V, D108N, H123Y, G125A, A142N, S146C, D147Y, R152H, R152P, E155V, I156F, K157N, K161T, based on amino acid sequence positions of E. coli TadA, and mutations in a homologous deaminase protein corresponding to the above. In some embodiments, the adenosine deaminase may comprise one or more of the mutations: D108N based on amino acid sequence positions of E. coli TadA, and mutations in a homologous deaminase protein corresponding to the above. In some embodiments, the adenosine deaminase may comprise one or more of the mutations: A106V, D108N, based on amino acid sequence positions of E. coli TadA, and mutations in a homologous deaminase protein corresponding to the above. In some embodiments, the adenosine deaminase may comprise one or more of the mutations: A106V, D108N, D147Y, E155V, based on amino acid sequence positions of E. coli TadA, and mutations in a homologous deaminase protein corresponding to the above. In some embodiments, the adenosine deaminase may comprise one or more of the mutations: A106V, D108N, based on amino acid sequence positions of E. coli TadA, and mutations in a homologous deaminase protein corresponding to the above. In some embodiments, the adenosine deaminase may comprise one or more of the mutations: A106V, D108N, D147Y, E155V, L84F, H123Y, I156F, based on amino acid sequence positions of E. coli TadA, and mutations in a homologous deaminase protein corresponding to the above. In some embodiments, the adenosine deaminase may comprise one or more of the mutations: A106V, D108N, D147Y, E155V, L84F, H123Y, I156F, A142N, based on amino acid sequence positions of E. coli TadA, and mutations in a homologous deaminase protein corresponding to the above. In some embodiments, the adenosine deaminase may comprise one or more of the mutations: A106V, D108N, D147Y, E155V, L84F, H123Y, I156F, H36L, R51L, S146C, K157N, based on amino acid sequence positions of E. coli TadA, and mutations in a homologous deaminase protein corresponding to the above. In some embodiments, the adenosine deaminase may comprise one or more of the mutations: A106V, D108N, D147Y, E155V, L84F, H123Y, I156F, H36L, R51L, S146C, K157N, P48S, based on amino acid sequence positions of E. coli TadA, and mutations in a homologous deaminase protein corresponding to the above. In some embodiments, the adenosine deaminase may comprise one or more of the mutations: A106V, D108N, D147Y, E155V, L84F, H123Y, I156F, H36L, R51L, S146C, K157N, P48S, A142N, based on amino acid sequence positions of E. coli TadA, and mutations in a homologous deaminase protein corresponding to the above. In some embodiments, the adenosine deaminase may comprise one or more of the mutations: A106V, D108N, D147Y, E155V, L84F, H123Y, I156F, H36L, R51L, S146C, K157N, P48S, W23R, P48A, based on amino acid sequence positions of E. coli TadA, and mutations in a homologous deaminase protein corresponding to the above. In some embodiments, the adenosine deaminase may comprise one or more of the mutations: A106V, D108N, D147Y, E155V, L84F, H123Y, I156F, H36L, R51L, S146C, K157N, P48S, W23R, P48A, A142N, based on amino acid sequence positions of E. coli TadA, and mutations in a homologous deaminase protein corresponding to the above. In some embodiments, the adenosine deaminase may comprise one or more of the mutations: A106V, D108N, D147Y, E155V, L84F, H123Y, I156F, H36L, R51L, S146C, K157N, P48S, W23R, P48A, R152P, based on amino acid sequence positions of E. coli TadA, and mutations in a homologous deaminase protein corresponding to the above. In some embodiments, the adenosine deaminase may comprise one or more of the mutations: A106V, D108N, D147Y, E155V, L84F, H123Y, I156F, H36L, R51L, S146C, K157N, P48S, W23R, P48A, R152P, A142N, based on amino acid sequence positions of E. coli TadA, and mutations in a homologous deaminase protein corresponding to the above.

Results suggest that A's opposite C's in the targeting window of the ADAR deaminase domain are preferentially edited over other bases. Additionally, A's base-paired with U's within a few bases of the targeted base show low levels of editing by CRISPR-Cas-ADAR fusions, suggesting that there is flexibility for the enzyme to edit multiple A's. These two observations suggest that multiple A's in the activity window of CRISPR-Cas-ADAR fusions could be specified for editing by mismatching all A's to be edited with C's. Accordingly, in certain embodiments, multiple A:C mismatches in the activity window are designed to create multiple A:I edits. In certain embodiments, to suppress potential off-target editing in the activity window, non-target A's are paired with A's or G's.

The terms “editing specificity” and “editing preference” are used interchangeably herein to refer to the extent of A-to-I editing at a particular adenosine site in a double-stranded substrate. In some embodiment, the substrate editing preference is determined by the 5′ nearest neighbor and/or the 3′ nearest neighbor of the target adenosine residue. In some embodiments, the adenosine deaminase has preference for the 5′ nearest neighbor of the substrate ranked as U>A>C>G (“>” indicates greater preference). In some embodiments, the adenosine deaminase has preference for the 3′ nearest neighbor of the substrate ranked as G>C˜A>U (“>” indicates greater preference; “˜” indicates similar preference). In some embodiments, the adenosine deaminase has preference for the 3′ nearest neighbor of the substrate ranked as G>C>U˜A (“>” indicates greater preference; “˜” indicates similar preference). In some embodiments, the adenosine deaminase has preference for the 3′ nearest neighbor of the substrate ranked as G>C>A>U (“>” indicates greater preference). In some embodiments, the adenosine deaminase has preference for the 3′ nearest neighbor of the substrate ranked as C˜G˜A>U (“>” indicates greater preference; “˜” indicates similar preference). In some embodiments, the adenosine deaminase has preference for a triplet sequence containing the target adenosine residue ranked as TAG>AAG>CAC>AAT>GAA>GAC (“>” indicates greater preference), the center A being the target adenosine residue.

In some embodiments, the substrate editing preference of an adenosine deaminase is affected by the presence or absence of a nucleic acid binding domain in the adenosine deaminase protein. In some embodiments, to modify substrate editing preference, the deaminase domain is connected with a double-strand RNA binding domain (dsRBD) or a double-strand RNA binding motif (dsRBM). In some embodiments, the dsRBD or dsRBM may be derived from an ADAR protein, such as hADAR1 or hADAR2. In some embodiments, a full length ADAR protein that comprises at least one dsRBD and a deaminase domain is used. In some embodiments, the one or more dsRBM or dsRBD is at the N-terminus of the deaminase domain. In other embodiments, the one or more dsRBM or dsRBD is at the C-terminus of the deaminase domain.

In some embodiments, the substrate editing preference of an adenosine deaminase is affected by amino acid residues near or in the active center of the enzyme. In some embodiments, to modify substrate editing preference, the adenosine deaminase may comprise one or more of the mutations: G336D, G487R, G487K, G487W, G487Y, E488Q, E488N, T490A, V493A, V493T, V493S, N597K, N597R, A589V, S599T, N613K, N613R, based on amino acid sequence positions of hADAR2-D, and mutations in a homologous ADAR protein corresponding to the above.

Particularly, in some embodiments, to reduce editing specificity, the adenosine deaminase can comprise one or more of mutations E488Q, V493A, N597K, N613K, based on amino acid sequence positions of hADAR2-D, and mutations in a homologous ADAR protein corresponding to the above. In some embodiments, to increase editing specificity, the adenosine deaminase can comprise mutation T490A.

In some embodiments, to increase editing preference for target adenosine (A) with an immediate 5′ G, such as substrates comprising the triplet sequence GAC, the center A being the target adenosine residue, the adenosine deaminase can comprise one or more of mutations G336D, E488Q, E488N, V493T, V493S, V493A, A589V, N597K, N597R, S599T, N613K, N613R, based on amino acid sequence positions of hADAR2-D, and mutations in a homologous ADAR protein corresponding to the above.

Particularly, in some embodiments, the adenosine deaminase comprises mutation E488Q or a corresponding mutation in a homologous ADAR protein for editing substrates comprising the following triplet sequences: GAC, GAA, GAU, GAG, CAU, AAU, UAC, the center A being the target adenosine residue.

In some embodiments, the adenosine deaminase comprises the wild-type amino acid sequence of hADAR1-D. In some embodiments, the adenosine deaminase comprises one or more mutations in the hADAR1-D sequence, such that the editing efficiency, and/or substrate editing preference of hADAR1-D is changed according to specific needs.

In some embodiments, the adenosine deaminase comprises a mutation at Glycine1007 of the hADAR1-D amino acid sequence, or a corresponding position in a homologous ADAR protein. In some embodiments, the glycine residue at position 1007 is replaced by a non-polar amino acid residue with relatively small side chains. For example, in some embodiments, the glycine residue at position 1007 is replaced by an alanine residue (G1007A). In some embodiments, the glycine residue at position 1007 is replaced by a valine residue (G1007V). In some embodiments, the glycine residue at position 1007 is replaced by an amino acid residue with relatively large side chains. In some embodiments, the glycine residue at position 1007 is replaced by an arginine residue (G1007R). In some embodiments, the glycine residue at position 1007 is replaced by a lysine residue (G1007K). In some embodiments, the glycine residue at position 1007 is replaced by a tryptophan residue (G1007W). In some embodiments, the glycine residue at position 1007 is replaced by a tyrosine residue (G1007Y). Additionally, in other embodiments, the glycine residue at position 1007 is replaced by a leucine residue (G1007L). In other embodiments, the glycine residue at position 1007 is replaced by a threonine residue (G1007T). In other embodiments, the glycine residue at position 1007 is replaced by a serine residue (G1007S).

In some embodiments, the adenosine deaminase comprises a mutation at glutamic acid1008 of the hADAR1-D amino acid sequence, or a corresponding position in a homologous ADAR protein. In some embodiments, the glutamic acid residue at position 1008 is replaced by a polar amino acid residue having a relatively large side chain. In some embodiments, the glutamic acid residue at position 1008 is replaced by a glutamine residue (E1008Q). In some embodiments, the glutamic acid residue at position 1008 is replaced by a histidine residue (E1008H). In some embodiments, the glutamic acid residue at position 1008 is replaced by an arginine residue (E1008R). In some embodiments, the glutamic acid residue at position 1008 is replaced by a lysine residue (E1008K). In some embodiments, the glutamic acid residue at position 1008 is replaced by a nonpolar or small polar amino acid residue. In some embodiments, the glutamic acid residue at position 1008 is replaced by a phenylalanine residue (E1008F). In some embodiments, the glutamic acid residue at position 1008 is replaced by a tryptophan residue (E1008W). In some embodiments, the glutamic acid residue at position 1008 is replaced by a glycine residue (E1008G). In some embodiments, the glutamic acid residue at position 1008 is replaced by an isoleucine residue (E1008I). In some embodiments, the glutamic acid residue at position 1008 is replaced by a valine residue (E1008V). In some embodiments, the glutamic acid residue at position 1008 is replaced by a proline residue (E1008P). In some embodiments, the glutamic acid residue at position 1008 is replaced by a serine residue (E1008S). In other embodiments, the glutamic acid residue at position 1008 is replaced by an asparagine residue (E1008N). In other embodiments, the glutamic acid residue at position 1008 is replaced by an alanine residue (E1008A). In other embodiments, the glutamic acid residue at position 1008 is replaced by a Methionine residue (E1008M). In some embodiments, the glutamic acid residue at position 1008 is replaced by a leucine residue (E1008L).

In some embodiments, to improve editing efficiency, the adenosine deaminase may comprise one or more of the mutations: E1007S, E1007A, E1007V, E1008Q, E1008R, E1008H, E1008M, E1008N, E1008K, based on amino acid sequence positions of hADAR1-D, and mutations in a homologous ADAR protein corresponding to the above.

In some embodiments, to reduce editing efficiency, the adenosine deaminase may comprise one or more of the mutations: E1007R, E1007K, E1007Y, E1007L, E1007T, E1008G, E1008I, E1008P, E1008V, E1008F, E1008W, E1008S, E1008N, E1008K, based on amino acid sequence positions of hADAR1-D, and mutations in a homologous ADAR protein corresponding to the above.

In some embodiments, the substrate editing preference, efficiency and/or selectivity of an adenosine deaminase is affected by amino acid residues near or in the active center of the enzyme. In some embodiments, the adenosine deaminase comprises a mutation at the glutamic acid 1008 position in hADAR1-D sequence, or a corresponding position in a homologous ADAR protein. In some embodiments, the mutation is E1008R, or a corresponding mutation in a homologous ADAR protein. In some embodiments, the E1008R mutant has an increased editing efficiency for target adenosine residue that has a mismatched G residue on the opposite strand.

In some embodiments, the adenosine deaminase protein further comprises or is connected to one or more double-stranded RNA (dsRNA) binding motifs (dsRBMs) or domains (dsRBDs) for recognizing and binding to double-stranded nucleic acid substrates. In some embodiments, the interaction between the adenosine deaminase and the double-stranded substrate is mediated by one or more additional protein factor(s), including a CRISPR/CAS protein factor. In some embodiments, the interaction between the adenosine deaminase and the double-stranded substrate is further mediated by one or more nucleic acid component(s), including a guide RNA.

In certain example embodiments, directed evolution may be used to design modified ADAR proteins capable of catalyzing additional reactions besides deamination of a adenine to a hypoxanthine.

### Modified Adenosine Deaminase Having C to U Deamination Activity

In certain example embodiments, directed evolution may be used to design modified ADAR proteins capable of catalyzing additional reactions besides deamination of an adenine to a hypoxanthine. For example, the modified ADAR protein may be capable of catalyzing deamination of a cytidine to a uracil. While not bound by a particular theory, mutations that improve C to U activity may alter the shape of the binding pocket to be more amenable to the smaller cytidine base.

In certain embodiments the adenosine deaminase is engineered to convert the activity to cytidine deaminase. Such engineered adenosine deaminase may also retain its adenosine deaminase activity, i.e., such mutated adenosine deaminase may have both adenosine deaminase and cytidine deaminase activities. Accordingly in some embodiments, the adenosine deaminase comprises one or more mutations in positions selected from E396, C451, V351, R455, T375, K376, S486, Q488, R510, K594, R348, G593, S397, H443, L444, Y445, F442, E438, T448, A353, V355, T339, P539, T339, P539, V525 I520, P462 and N579. In particular embodiments, the adenosine deaminase comprises one or more mutations in a position selected from V351, L444, V355, V525 and 1520. In some embodiments, the adenosine deaminase may comprise one or more of mutations at E488, V351, S486, T375, S370, P462, N597, based on amino acid sequence positions of hADAR2-D, and mutations in a homologous ADAR protein corresponding to the above.

In some embodiments, the adenosine deaminase may comprise one or more of the mutations: E488Q based on amino acid sequence positions of hADAR2-D, and mutations in a homologous ADAR protein corresponding to the above. In some embodiments, the adenosine deaminase may comprise one or more of the mutations: E488Q, V351G, based on amino acid sequence positions of hADAR2-D, and mutations in a homologous ADAR protein corresponding to the above. In some embodiments, the adenosine deaminase may comprise one or more of the mutations: E488Q, V351G, S486A, based on amino acid sequence positions of hADAR2-D, and mutations in a homologous ADAR protein corresponding to the above. In some embodiments, the adenosine deaminase may comprise one or more of the mutations: E488Q, V351G, S486A, T375S, based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, P462A, based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, P462A, N597I, based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, P462A, N597I, L332I, based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, P462A, N597I, L332I, I398V, based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, P462A, N597I, L332I, I398V, K350I, based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, P462A, N597I, L332I, I398V, K350I, M383L, based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, P462A, N597I, L332I, I398V, K350I, M383L, D619G, based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, P462A, N597I, L332I, I398V, K350I, M383L, D619G, S582T, based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, P462A, N597I, L332I, I398V, K350I, M383L, D619G, S582T, V440I based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, P462A, N597I, L332I, I398V, K350I, M383L, D619G, S582T, V440I, S495N based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, P462A, N597I, L332I, I398V, K350I, M383L, D619G, S582T, V440I, S495N, K418E based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, P462A, N597I, L332I, I398V, K350I, M383L, D619G, S582T, V440I, S495N, K418E, S661T based on amino acid sequence positions of hADAR2-D, and mutations in a homologous ADAR protein corresponding to the above. In some examples, provided herein includes a mutated adenosine deaminase e.g., an adenosine deaminase comprising one or more mutations of E488Q, V351G, S486A, T375S, S370C, P462A, N597I, L332I, 1398V, K350I, M383L, D619G, S582T, V440I, S495N, K418E, S661T, fused with a dead CRISPR-Cas protein or CRISPR-Cas nickase. In a particular example, provided herein includes a mutated adenosine deaminase e.g., an adenosine deaminase comprising E488Q, V351G, S486A, T375S, S370C, P462A, N597I, L332I, I398V, K350I, M383L, D619G, S582T, V440I, S495N, K418E, and S661T, fused with a dead CRISPR-Cas protein or a CRISPR-Cas nickase.

In some embodiments, the modified adenosine deaminase having C-to-U deamination activity comprises a mutation at any one or more of positions V351, T375, R455, and E488 of the hADAR2-D amino acid sequence, or a corresponding position in a homologous ADAR protein. In some embodiments, the adenosine deaminase comprises mutation E488Q. In some embodiments, the adenosine deaminase comprises one or more of mutations selected from V351I, V351L, V351F, V351M, V351C, V351A, V351G, V351P, V351T, V351S, V351Y, V351W, V351Q, V351N, V351H, V351E, V351D, V351K, V351R, T375I, T375L, T375V, T375F, T375M, T375C, T375A, T375G, T375P, T375S, T375Y, T375W, T375Q, T375N, T375H, T375E, T375D, T375K, T375R, R455I, R455L, R455V, R455F, R455M, R455C, R455A, R455G, R455P, R455T, R455S, R455Y, R455W, R455Q, R455N, R455H, R455E, R455D, R455K. In some embodiments, the adenosine deaminase comprises mutation E488Q, and further comprises one or more of mutations selected from V351I, V351L, V351F, V351M, V351C, V351A, V351G, V351P, V351T, V351S, V351Y, V351W, V351Q, V351N, V351H, V351E, V351D, V351K, V351R, T375I, T375L, T375V, T375F, T375M, T375C, T375A, T375G, T375P, T375S, T375Y, T375W, T375Q, T375N, T375H, T375E, T375D, T375K, T375R, R455I, R455L, R455V, R455F, R455M, R455C, R455A, R455G, R455P, R455T, R455S, R455Y, R455W, R455Q, R455N, R455H, R455E, R455D, R455K.

In connection with the aforementioned modified ADAR protein having C-to-U deamination activity, the invention described herein also relates to a method for deaminating a C in a target RNA sequence of interest, comprising delivering to a target RNA or DNA an AD-functionalized composition disclosed herein.

In certain example embodiments, the method for deaminating a C in a target RNA sequence comprising delivering to said target RNA: (a) a catalytically inactive (dead) Cas; (b) a guide molecule which comprises a guide sequence linked to a direct repeat sequence; and (c) a modified ADAR protein having C-to-U deamination activity or catalytic domain thereof; wherein said modified ADAR protein or catalytic domain thereof is covalently or non-covalently linked to said dead Cas protein or said guide molecule or is adapted to link thereto after delivery; wherein guide molecule forms a complex with said dead Cas protein and directs said complex to bind said target RNA sequence of interest; wherein said guide sequence is capable of hybridizing with a target sequence comprising said C to form an RNA duplex; wherein, optionally, said guide sequence comprises a non-pairing A or U at a position corresponding to said C resulting in a mismatch in the RNA duplex formed; and wherein said modified ADAR protein or catalytic domain thereof deaminates said C in said RNA duplex.

In connection with the aforementioned modified ADAR protein having C-to-U deamination activity, the invention described herein further relates to an engineered, non-naturally occurring system suitable for deaminating a C in a target locus of interest, comprising: (a) a guide molecule which comprises a guide sequence linked to a direct repeat sequence, or a nucleotide sequence encoding said guide molecule; (b) a catalytically inactive CRISPR-Cas protein, or a nucleotide sequence encoding said catalytically inactive CRISPR-Cas protein; (c) a modified ADAR protein having C-to-U deamination activity or catalytic domain thereof, or a nucleotide sequence encoding said modified ADAR protein or catalytic domain thereof; wherein said modified ADAR protein or catalytic domain thereof is covalently or non-covalently linked to said CRISPR-Cas protein or said guide molecule or is adapted to link thereto after delivery; wherein said guide sequence is capable of hybridizing with a target RNA sequence comprising a C to form an RNA duplex; wherein, optionally, said guide sequence comprises a non-pairing A or U at a position corresponding to said C resulting in a mismatch in the RNA duplex formed; wherein, optionally, the system is a vector system comprising one or more vectors comprising: (a) a first regulatory element operably linked to a nucleotide sequence encoding said guide molecule which comprises said guide sequence, (b) a second regulatory element operably linked to a nucleotide sequence encoding said catalytically inactive CRISPR-Cas protein; and (c) a nucleotide sequence encoding a modified ADAR protein having C-to-U deamination activity or catalytic domain thereof which is under control of said first or second regulatory element or operably linked to a third regulatory element; wherein, if said nucleotide sequence encoding a modified ADAR protein or catalytic domain thereof is operably linked to a third regulatory element, said modified ADAR protein or catalytic domain thereof is adapted to link to said guide molecule or said CRISPR-Cas protein after expression; wherein components (a), (b) and (c) are located on the same or different vectors of the system, optionally wherein said first, second, and/or third regulatory element is an inducible promoter.

In an embodiment, the substrate of the adenosine deaminase is an RNA/DNA heteroduplex formed upon binding of the guide molecule to its DNA target which then forms the CRISPR-Cas complex with the CRISPR-Cas enzyme. The RNA/DNA or DNA/RNA heteroduplex is also referred to herein as the “RNA/DNA hybrid”, “DNA/RNA hybrid” or “double-stranded substrate”.

According to the present disclosure, the substrate of the adenosine deaminase is an RNA/DNAn RNA duplex formed upon binding of the guide molecule to its DNA target which then forms the CRISPR-Cas complex with the CRISPR-Cas enzyme. The substrate of the adenosine deaminase can also be an RNA/RNA duplex formed upon binding of the guide molecule to its RNA target which then forms the CRISPR-Cas complex with the CRISPR-Cas enzyme. The RNA/DNA or DNA/RNAn RNA duplex is also referred to herein as the “RNA/DNA hybrid”, “DNA/RNA hybrid” or “double-stranded substrate”. The particular features of the guide molecule and CRISPR-Cas enzyme are detailed below.

The term “editing selectivity” as used herein refers to the fraction of all sites on a double-stranded substrate that is edited by an adenosine deaminase. Without being bound by theory, it is contemplated that editing selectivity of an adenosine deaminase is affected by the double-stranded substrate's length and secondary structures, such as the presence of mismatched bases, bulges and/or internal loops.

In some embodiments, when the substrate is a perfectly base-paired duplex longer than 50 bp, the adenosine deaminase may be able to deaminate multiple adenosine residues within the duplex (e.g., 50% of all adenosine residues). In some embodiments, when the substrate is shorter than 50 bp, the editing selectivity of an adenosine deaminase is affected by the presence of a mismatch at the target adenosine site. Particularly, in some embodiments, adenosine (A) residue having a mismatched cytidine (C) residue on the opposite strand is deaminated with high efficiency. In some embodiments, adenosine (A) residue having a mismatched guanosine (G) residue on the opposite strand is skipped without editing.

In particular embodiments, the adenosine deaminase protein or catalytic domain thereof is delivered to the cell or expressed within the cell as a separate protein, but is modified so as to be able to link to either the Cas protein or the guide molecule. In particular embodiments, this is ensured by the use of orthogonal RNA-binding protein or adaptor protein/aptamer combinations that exist within the diversity of bacteriophage coat proteins. Examples of such coat proteins include but are not limited to: MS2, QP, F2, GA, fr, JP501, M12, R17, BZ13, JP34, JP500, KU1, M11, MX1, TW18, VK, SP, FI, ID2, NL95, TW19, AP205, QCb5, QCb8r, Cb12r, QCb23r, 7s and PRR1. Aptamers can be naturally occurring or synthetic oligonucleotides that have been engineered through repeated rounds of in vitro selection or SELEX (systematic evolution of ligands by exponential enrichment) to bind to a specific target.

In particular embodiments, the guide molecule is provided with one or more distinct RNA loop(s) or distinct sequence(s) that can recruit an adaptor protein. A guide molecule may be extended, without colliding with the Cas protein by the insertion of distinct RNA loop(s) or distinct sequence(s) that may recruit adaptor proteins that can bind to the distinct RNA loop(s) or distinct sequence(s). Examples of modified guides and their use in recruiting effector domains to the Cas complex are provided in Konermann (Nature 2015, 517(7536): 583-588). In particular embodiments, the aptamer is a minimal hairpin aptamer which selectively binds dimerized MS2 bacteriophage coat proteins in mammalian cells and is introduced into the guide molecule, such as in the stemloop and/or in a tetraloop. In these embodiments, the adenosine deaminase protein is fused to MS2. The adenosine deaminase protein is then co-delivered together with the Cas protein and corresponding guide RNA.

In some embodiments, the Cas-ADAR base editing system described herein comprises (a) a Cas protein, which is catalytically inactive or a nickase; (b) a guide molecule which comprises a guide sequence; and (c) an adenosine deaminase protein or catalytic domain thereof, wherein the adenosine deaminase protein or catalytic domain thereof is covalently or non-covalently linked to the Cas protein or the guide molecule or is adapted to link thereto after delivery; wherein the guide sequence is substantially complementary to the target sequence but comprises a non-pairing C corresponding to the A being targeted for deamination, resulting in a A-C mismatch in a DNA-RNA or RNA-RNA duplex formed by the guide sequence and the target sequence. For application in eukaryotic cells, the Cas protein and/or the adenosine deaminase are preferably NLS-tagged.

In some embodiments, the components (a), (b) and (c) are delivered to the cell as a ribonucleoprotein complex. The ribonucleoprotein complex can be delivered via one or more lipid nanoparticles.

In some embodiments, the components (a), (b) and (c) are delivered to the cell as one or more RNA molecules, such as one or more guide RNAs and one or more mRNA molecules encoding the Cas protein, the adenosine deaminase protein, and optionally the adaptor protein. The RNA molecules can be delivered via one or more lipid nanoparticles.

In some embodiments, the components (a), (b) and (c) are delivered to the cell as one or more DNA molecules. In some embodiments, the one or more DNA molecules are comprised within one or more vectors such as viral vectors (e.g., AAV). In some embodiments, the one or more DNA molecules comprise one or more regulatory elements operably configured to express the Cas protein, the guide molecule, and the adenosine deaminase protein or catalytic domain thereof, optionally wherein the one or more regulatory elements comprise inducible promoters.

In some embodiments of the guide molecule is capable of hybridizing with a target sequence comprising the Adenine to be deaminated within a first DNA strand or a RNA strand at the target locus to form a DNA-RNA or RNA-RNA duplex which comprises a non-pairing Cytosine opposite to said Adenine. Upon duplex formation, the guide molecule forms a complex with the Cas protein and directs the complex to bind said first DNA strand or said RNA strand at the target locus of interest. Details on the aspect of the guide of the Cas-ADAR base editing system are provided herein below.

In some embodiments, a Cas guide RNA having a canonical length (e.g., about 20 nt for AacCas) is used to form a DNA-RNA or RNA-RNA duplex with the target DNA or RNA. In some embodiments, a Cas guide molecule longer than the canonical length (e.g., >20 nt for AacCas) is used to form a DNA-RNA or RNA-RNA duplex with the target DNA or RNA including outside of the Cas-guide RNA-target DNA complex. In certain example embodiments, the guide sequence has a length of about 29-53 nt capable of forming a DNA-RNA or RNA-RNA duplex with said target sequence. In certain other example embodiments, the guide sequence has a length of about 40-50 nt capable of forming a DNA-RNA or RNA-RNA duplex duplex with said target sequence. In certain example embodiments, the distance between said non-pairing C and the 5′ end of said guide sequence is 20-30 nucleotides. In certain example embodiments, the distance between said non-pairing C and the 3′ end of said guide sequence is 20-30 nucleotides.

In at least a first design, the Cas-ADAR system comprises (a) an adenosine deaminase fused or linked to a Cas protein, wherein the Cas protein is catalytically inactive or a nickase, and (b) a guide molecule comprising a guide sequence designed to introduce a A-C mismatch in a DNA-RNA or RNA-RNA duplex formed between the guide sequence and the target sequence. In some embodiments, the Cas protein and/or the adenosine deaminase are NLS-tagged, on either the N- or C-terminus or both.

In at least a second design, the Cas-ADAR system comprises (a) a Cas protein that is catalytically inactive or a nickase, (b) a guide molecule comprising a guide sequence designed to introduce a A-C mismatch in a DNA-RNA or RNA-RNA duplex formed between the guide sequence and the target sequence, and an aptamer sequence (e.g., MS2 RNA motif or PP7 RNA motif) capable of binding to an adaptor protein (e.g., MS2 coating protein or PP7 coat protein), and (c) an adenosine deaminase fused or linked to an adaptor protein, wherein the binding of the aptamer and the adaptor protein recruits the adenosine deaminase to the DNA-RNA or RNA-RNA duplex formed between the guide sequence and the target sequence for targeted deamination at the A of the A-C mismatch. In some embodiments, the adaptor protein and/or the adenosine deaminase are NLS-tagged, on either the N- or C-terminus or both. The Cas protein can also be NLS-tagged.

The use of different aptamers and corresponding adaptor proteins also allows orthogonal gene editing to be implemented. In one example in which adenosine deaminase are used in combination with cytidine deaminase for orthogonal gene editing/deamination, sgRNA targeting different loci are modified with distinct RNA loops in order to recruit MS2-adenosine deaminase and PP7-cytidine deaminase (or PP7-adenosine deaminase and MS2-cytidine deaminase), respectively, resulting in orthogonal deamination of A or C at the target loci of interested, respectively. PP7 is the RNA-binding coat protein of the bacteriophage Pseudomonas. Like MS2, it binds a specific RNA sequence and secondary structure. The PP7 RNA-recognition motif is distinct from that of MS2. Consequently, PP7 and MS2 can be multiplexed to mediate distinct effects at different genomic loci simultaneously. For example, an sgRNA targeting locus A can be modified with MS2 loops, recruiting MS2-adenosine deaminase, while another sgRNA targeting locus B can be modified with PP7 loops, recruiting PP7-cytidine deaminase. In the same cell, orthogonal, locus-specific modifications are thus realized. This principle can be extended to incorporate other orthogonal RNA-binding proteins.

In at least a third design, the Cas-ADAR CRISPR system comprises (a) an adenosine deaminase inserted into an internal loop or unstructured region of a Cas protein, wherein the Cas protein is catalytically inactive or a nickase, and (b) a guide molecule comprising a guide sequence designed to introduce a A-C mismatch in a DNA-RNA or RNA-RNA duplex formed between the guide sequence and the target sequence.

Cas protein split sites that are suitable for insertion of adenosine deaminase can be identified with the help of a crystal structure. For example, with respect to AacCas mutants, it should be readily apparent what the corresponding position for, for example, a sequence alignment. For other Cas protein one can use the crystal structure of an ortholog if a relatively high degree of homology exists between the ortholog and the intended Cas protein.

The split position may be located within a region or loop. Preferably, the split position occurs where an interruption of the amino acid sequence does not result in the partial or full destruction of a structural feature (e.g. alpha-helixes or β-sheets). Unstructured regions (regions that did not show up in the crystal structure because these regions are not structured enough to be “frozen” in a crystal) are often preferred options. Splits in all unstructured regions that are exposed on the surface of Cas are envisioned in the practice of the invention. The positions within the unstructured regions or outside loops may not need to be exactly the numbers provided above, but may vary by, for example 1, 2, 3, 4, 5, 6, 7, 8, 9, or even 10 amino acids either side of the position given above, depending on the size of the loop, so long as the split position still falls within an unstructured region of outside loop.

The Cas-ADAR system described herein can be used to target a specific Adenine within a DNA sequence for deamination. For example, the guide molecule can form a complex with the Cas protein and directs the complex to bind a target sequence at the target locus of interest. Because the guide sequence is designed to have a non-pairing C, the heteroduplex formed between the guide sequence and the target sequence comprises a A-C mismatch, which directs the adenosine deaminase to contact and deaminate the A opposite to the non-pairing C, converting it to a Inosine (I). Since Inosine (I) base pairs with C and functions like G in cellular process, the targeted deamination of A described herein are useful for correction of undesirable G-A and C-T mutations, as well as for obtaining desirable A-G and T-C mutations.

### Base Excision Repair Inhibitor

In some embodiments, the AD-functionalized CRISPR system further comprises a base excision repair (BER) inhibitor. Without wishing to be bound by any particular theory, cellular DNA-repair response to the presence of I:T pairing may be responsible for a decrease in nucleobase editing efficiency in cells. Alkyladenine DNA glycosylase (also known as DNA-3-methyladenine glycosylase, 3-alkyladenine DNA glycosylase, or N-methylpurine DNA glycosylase) catalyzes removal of hypoxanthine from DNA in cells, which may initiate base excision repair, with reversion of the I:T pair to a A:T pair as outcome.

In some embodiments, the BER inhibitor is an inhibitor of alkyladenine DNA glycosylase. In some embodiments, the BER inhibitor is an inhibitor of human alkyladenine DNA glycosylase. In some embodiments, the BER inhibitor is a polypeptide inhibitor. In some embodiments, the BER inhibitor is a protein that binds hypoxanthine. In some embodiments, the BER inhibitor is a protein that binds hypoxanthine in DNA. In some embodiments, the BER inhibitor is a catalytically inactive alkyladenine DNA glycosylase protein or binding domain thereof. In some embodiments, the BER inhibitor is a catalytically inactive alkyladenine DNA glycosylase protein or binding domain thereof that does not excise hypoxanthine from the DNA. Other proteins that are capable of inhibiting (e.g., sterically blocking) an alkyladenine DNA glycosylase base-excision repair enzyme are within the scope of this disclosure. Additionally, any proteins that block or inhibit base-excision repair as also within the scope of this disclosure.

Without wishing to be bound by any particular theory, base excision repair may be inhibited by molecules that bind the edited strand, block the edited base, inhibit alkyladenine DNA glycosylase, inhibit base excision repair, protect the edited base, and/or promote fixing of the non-edited strand. It is believed that the use of the BER inhibitor described herein can increase the editing efficiency of an adenosine deaminase that is capable of catalyzing a A to I change.

Accordingly, in the first design of the AD-functionalized CRISPR system discussed above, the CRISPR-Cas protein or the adenosine deaminase can be fused to or linked to a BER inhibitor (e.g., an inhibitor of alkyladenine DNA glycosylase). In some embodiments, the BER inhibitor can be comprised in one of the following structures (nCas=Cas nickase; dCas=dead Cas): [AD]-[optional linker]-[nCas/dCas]-[optional linker]-[BER inhibitor]; [AD]-[optional linker]-[BER inhibitor]-[optional linker]-[nCas/dCas]; [BER inhibitor]-[optional linker]-[AD]-[optional linker]-[nCas/dCas]; [BER inhibitor]-[optional linker]-[nCas/dCas]-[optional linker]-[AD]; [nCas/dCas]-[optional linker]-[AD]-[optional linker]-[BER inhibitor]; [nCas/dCas]-[optional linker]-[BER inhibitor]-[optional linker]-[AD].

Similarly, in the second design of the AD-functionalized CRISPR system discussed above, the CRISPR-Cas protein, the adenosine deaminase, or the adaptor protein can be fused to or linked to a BER inhibitor (e.g., an inhibitor of alkyladenine DNA glycosylase). In some embodiments, the BER inhibitor can be comprised in one of the following structures (nCas=Cas nickase; dCas=dead Cas): [nCas/dCas]-[optional linker]-[BER inhibitor]; [BER inhibitor]-[optional linker]-[nCas/dCas]; [AD]-[optional linker]-[Adaptor]-[optional linker]-[BER inhibitor]; [AD]-[optional linker]-[BER inhibitor]-[optional linker]-[Adaptor]; [BER inhibitor]-[optional linker]-[AD]-[optional linker]-[Adaptor]; [BER inhibitor]-[optional linker]-[Adaptor]-[optional linker]-[AD]; [Adaptor]-[optional linker]-[AD]-[optional linker]-[BER inhibitor]; [Adaptor]-[optional linker]-[BER inhibitor]-[optional linker]-[AD].

In the third design of the AD-functionalized CRISPR system discussed above, the BER inhibitor can be inserted into an internal loop or unstructured region of a CRISPR-Cas protein.

### Cytidine Deaminase

In some embodiments, the deaminase is a cytidine deaminase. The term “cytidine deaminase” or “cytidine deaminase protein” or “cytidine deaminase activity” as used herein refers to a protein, a polypeptide, or one or more functional domain(s) of a protein or a polypeptide that is capable of catalyzing a hydrolytic deamination reaction that converts an cytosine (or an cytosine moiety of a molecule) to an uracil (or a uracil moiety of a molecule), as shown below. In some embodiments, the cytosine-containing molecule is an cytidine (C), and the uracil-containing molecule is an uridine (U). The cytosine-containing molecule can be deoxyribonucleic acid (DNA) or ribonucleic acid (RNA). In certain examples, a cytidine deaminase may be a cytidine deaminase acting on RNA (CDAR).

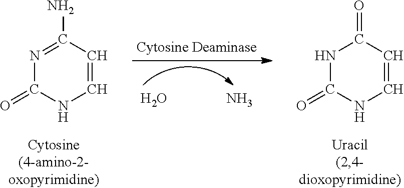

According to the present disclosure, cytidine deaminases that can be used in connection with the present disclosure include, but are not limited to, members of the enzyme family known as apolipoprotein B mRNA-editing complex (APOBEC) family deaminase, an activation-induced deaminase (AID), or a cytidine deaminase 1 (CDA1). In particular embodiments, the deaminase in an APOBEC1 deaminase, an APOBEC2 deaminase, an APOBEC3A deaminase, an APOBEC3B deaminase, an APOBEC3C deaminase, and APOBEC3D deaminase, an APOBEC3E deaminase, an APOBEC3F deaminase an APOBEC3G deaminase, an APOBEC3H deaminase, or an APOBEC4 deaminase.

In the methods and systems of the present invention, the cytidine deaminase or engineered adenosine deaminase with cytidine deaminase activity is capable of targeting Cytosine in a DNA single strand. In certain example embodiments the cytidine deaminase activity may edit on a single strand present outside of the binding component e.g. bound CRISPR-Cas. In other example embodiments, the cytidine deaminase may edit at a localized bubble, such as a localized bubble formed by a mismatch at the target edit site but the guide sequence. In certain example embodiments the cytidine deaminase may contain mutations that help focus the area of activity such as those disclosed in Kim et al., Nature Biotechnology (2017) 35(4):371-377 (doi:10.1038/nbt.3803.

In some embodiments, the cytidine deaminase is derived from one or more metazoa species, including but not limited to, mammals, birds, frogs, squids, fish, flies and worms. In some embodiments, the cytidine deaminase is a human, primate, cow, dog rat or mouse cytidine deaminase.

In some embodiments, the cytidine deaminase is a human APOBEC, including hAPOBEC1 or hAPOBEC3. In some embodiments, the cytidine deaminase is a human AID.

In some embodiments, the cytidine deaminase protein recognizes and converts one or more target cytosine residue(s) in a single-stranded bubble of a RNA duplex into uracil residues (s). In some embodiments, the cytidine deaminase protein recognizes a binding window on the single-stranded bubble of a RNA duplex. In some embodiments, the binding window contains at least one target cytosine residue(s). In some embodiments, the binding window is in the range of about 3 bp to about 100 bp. In some embodiments, the binding window is in the range of about 5 bp to about 50 bp. In some embodiments, the binding window is in the range of about 10 bp to about 30 bp. In some embodiments, the binding window is about 1 bp, 2 bp, 3 bp, 5 bp, 7 bp, 10 bp, 15 bp, 20 bp, 25 bp, 30 bp, 40 bp, 45 bp, 50 bp, 55 bp, 60 bp, 65 bp, 70 bp, 75 bp, 80 bp, 85 bp, 90 bp, 95 bp, or 100 bp.

In some embodiments, the cytidine deaminase protein comprises one or more deaminase domains. Not intended to be bound by theory, it is contemplated that the deaminase domain functions to recognize and convert one or more target cytosine (C) residue(s) contained in a single-stranded bubble of a RNA duplex into (an) uracil (U) residue (s). In some embodiments, the deaminase domain comprises an active center. In some embodiments, the active center comprises a zinc ion. In some embodiments, amino acid residues in or near the active center interact with one or more nucleotide(s) 5′ to a target cytosine residue. In some embodiments, amino acid residues in or near the active center interact with one or more nucleotide(s) 3′ to a target cytosine residue.

In some embodiments, the cytidine deaminase comprises human APOBEC1 full protein (hAPOBEC1) or the deaminase domain thereof (hAPOBEC1-D) or a C-terminally truncated version thereof (hAPOBEC-T). In some embodiments, the cytidine deaminase is an APOBEC family member that is homologous to hAPOBEC1, hAPOBEC-D or hAPOBEC-T. In some embodiments, the cytidine deaminase comprises human AID1 full protein (hAID) or the deaminase domain thereof (hAID-D) or a C-terminally truncated version thereof (hAID-T). In some embodiments, the cytidine deaminase is an AID family member that is homologous to hAID, hAID-D or hAID-T. In some embodiments, the hAID-T is a hAID which is C-terminally truncated by about 20 amino acids.

In some embodiments, the cytidine deaminase comprises the wild-type amino acid sequence of a cytosine deaminase. In some embodiments, the cytidine deaminase comprises one or more mutations in the cytosine deaminase sequence, such that the editing efficiency, and/or substrate editing preference of the cytosine deaminase is changed according to specific needs.

Certain mutations of APOBEC1 and APOBEC3 proteins have been described in Kim et al., Nature Biotechnology (2017) 35(4):371-377 (doi:10.1038/nbt.3803); and Harris et al. Mol. Cell (2002) 10:1247-1253, each of which is incorporated herein by reference in its entirety.

In some embodiments, the cytidine deaminase is an APOBEC1 deaminase comprising one or more mutations at amino acid positions corresponding to W90, R118, H121, H122, R126, or R132 in rat APOBEC1, or an APOBEC3G deaminase comprising one or more mutations at amino acid positions corresponding to W285, R313, D316, D317X, R320, or R326 in human APOBEC3G.

In some embodiments, the cytidine deaminase comprises a mutation at tryptophane90 of the rat APOBEC1 amino acid sequence, or a corresponding position in a homologous APOBEC protein, such as tryptophane285 of APOBEC3G. In some embodiments, the tryptophan residue at position 90 is replaced by an tyrosine or phenylalanine residue (W90Y or W90F).

In some embodiments, the cytidine deaminase comprises a mutation at Arginine118 of the rat APOBEC1 amino acid sequence, or a corresponding position in a homologous APOBEC protein. In some embodiments, the arginine residue at position 118 is replaced by an alanine residue (R118A).

In some embodiments, the cytidine deaminase comprises a mutation at Histidine121 of the rat APOBEC1 amino acid sequence, or a corresponding position in a homologous APOBEC protein. In some embodiments, the histidine residue at position 121 is replaced by an arginine residue (H121R).

In some embodiments, the cytidine deaminase comprises a mutation at Histidine122 of the rat APOBEC1 amino acid sequence, or a corresponding position in a homologous APOBEC protein. In some embodiments, the histidine residue at position 122 is replaced by an arginine residue (H122R).

In some embodiments, the cytidine deaminase comprises a mutation at Arginine126 of the rat APOBEC1 amino acid sequence, or a corresponding position in a homologous APOBEC protein, such as Arginine320 of APOBEC3G. In some embodiments, the arginine residue at position 126 is replaced by an alanine residue (R126A) or by a glutamic acid (R126E).

In some embodiments, the cytidine deaminase comprises a mutation at arginine132 of the APOBEC1 amino acid sequence, or a corresponding position in a homologous APOBEC protein. In some embodiments, the arginine residue at position 132 is replaced by a glutamic acid residue (R132E).

In some embodiments, to narrow the width of the editing window, the cytidine deaminase may comprise one or more of the mutations: W90Y, W90F, R126E and R132E, based on amino acid sequence positions of rat APOBEC1, and mutations in a homologous APOBEC protein corresponding to the above.

In some embodiments, to reduce editing efficiency, the cytidine deaminase may comprise one or more of the mutations: W90A, R118A, R132E, based on amino acid sequence positions of rat APOBEC1, and mutations in a homologous APOBEC protein corresponding to the above. In particular embodiments, it can be of interest to use a cytidine deaminase enzyme with reduced efficacy to reduce off-target effects.

In some embodiments, the cytidine deaminase is wild-type rat APOBEC1 (rAPOBEC1, or a catalytic domain thereof. In some embodiments, the cytidine deaminase comprises one or more mutations in the rAPOBEC1 sequence, such that the editing efficiency, and/or substrate editing preference of rAPOBEC1 is changed according to specific needs.

In some embodiments, the cytidine deaminase is wild-type human APOBEC1 (hAPOBEC1) or a catalytic domain thereof. In some embodiments, the cytidine deaminase comprises one or more mutations in the hAPOBEC1 sequence, such that the editing efficiency, and/or substrate editing preference of hAPOBEC 1 is changed according to specific needs.

In some embodiments, the cytidine deaminase is wild-type human APOBEC3G (hAPOBEC3G) or a catalytic domain thereof. In some embodiments, the cytidine deaminase comprises one or more mutations in the hAPOBEC3G sequence, such that the editing efficiency, and/or substrate editing preference of hAPOBEC3G is changed according to specific needs.

In some embodiments, the cytidine deaminase is wild-type Petromyzon marinus CDA1 (pmCDA1) or a catalytic domain thereof. In some embodiments, the cytidine deaminase comprises one or more mutations in the pmCDA1 sequence, such that the editing efficiency, and/or substrate editing preference of pmCDA1 is changed according to specific needs.

In some embodiments, the cytidine deaminase is wild-type human AID (hAID) or a catalytic domain thereof. In some embodiments, the cytidine deaminase comprises one or more mutations in the pmCDA1 sequence, such that the editing efficiency, and/or substrate editing preference of pmCDA1 is changed according to specific needs.

In some embodiments, the cytidine deaminase is truncated version of hAID (hAID-DC) or a catalytic domain thereof. In some embodiments, the cytidine deaminase comprises one or more mutations in the hAID-DC sequence, such that the editing efficiency, and/or substrate editing preference of hAID-DC is changed according to specific needs.

Additional embodiments of the cytidine deaminase are disclosed in WO WO2017/070632, titled “Nucleobase Editor and Uses Thereof,” which is incorporated herein by reference in its entirety.

In some embodiments, the cytidine deaminase has an efficient deamination window that encloses the nucleotides susceptible to deamination editing. Accordingly, in some embodiments, the “editing window width” refers to the number of nucleotide positions at a given target site for which editing efficiency of the cytidine deaminase exceeds the half-maximal value for that target site. In some embodiments, the cytidine deaminase has an editing window width in the range of about 1 to about 6 nucleotides. In some embodiments, the editing window width of the cytidine deaminase is 1, 2, 3, 4, 5, or 6 nucleotides.

Not intended to be bound by theory, it is contemplated that in some embodiments, the length of the linker sequence affects the editing window width. In some embodiments, the editing window width increases (e.g., from about 3 to about 6 nucleotides) as the linker length extends (e.g., from about 3 to about 21 amino acids). In a non-limiting example, a 16-residue linker offers an efficient deamination window of about 5 nucleotides. In some embodiments, the length of the guide RNA affects the editing window width. In some embodiments, shortening the guide RNA leads to a narrowed efficient deamination window of the cytidine deaminase.

In some embodiments, mutations to the cytidine deaminase affect the editing window width. In some embodiments, the cytidine deaminase component of the CD-functionalized CRISPR system comprises one or more mutations that reduce the catalytic efficiency of the cytidine deaminase, such that the deaminase is prevented from deamination of multiple cytidines per DNA binding event. In some embodiments, tryptophan at residue 90 (W90) of APOBEC1 or a corresponding tryptophan residue in a homologous sequence is mutated. In some embodiments, the catalytically inactive CRISPR-Cas is fused to or linked to an APOBEC1 mutant that comprises a W90Y or W90F mutation. In some embodiments, tryptophan at residue 285 (W285) of APOBEC3G, or a corresponding tryptophan residue in a homologous sequence is mutated. In some embodiments, the catalytically inactive CRISPR-Cas is fused to or linked to an APOBEC3G mutant that comprises a W285Y or W285F mutation.

In some embodiments, the cytidine deaminase component of CD-functionalized CRISPR system comprises one or more mutations that reduce tolerance for non-optimal presentation of a cytidine to the deaminase active site. In some embodiments, the cytidine deaminase comprises one or more mutations that alter substrate binding activity of the deaminase active site. In some embodiments, the cytidine deaminase comprises one or more mutations that alter the conformation of DNA to be recognized and bound by the deaminase active site. In some embodiments, the cytidine deaminase comprises one or more mutations that alter the substrate accessibility to the deaminase active site. In some embodiments, arginine at residue 126 (R126) of APOBEC1 or a corresponding arginine residue in a homologous sequence is mutated. In some embodiments, the catalytically inactive CRISPR-Cas is fused to or linked to an APOBEC1 that comprises a R126A or R126E mutation. In some embodiments, tryptophan at residue 320 (R320) of APOBEC3G, or a corresponding arginine residue in a homologous sequence is mutated. In some embodiments, the catalytically inactive CRISPR-Cas is fused to or linked to an APOBEC3G mutant that comprises a R320A or R320E mutation. In some embodiments, arginine at residue 132 (R132) of APOBEC1 or a corresponding arginine residue in a homologous sequence is mutated. In some embodiments, the catalytically inactive CRISPR-Cas is fused to or linked to an APOBEC1 mutant that comprises a R132E mutation.

In some embodiments, the APOBEC1 domain of the CD-functionalized CRISPR system comprises one, two, or three mutations selected from W90Y, W90F, R126A, R126E, and R132E. In some embodiments, the APOBEC1 domain comprises double mutations of W90Y and R126E. In some embodiments, the APOBEC1 domain comprises double mutations of W90Y and R132E. In some embodiments, the APOBEC1 domain comprises double mutations of R126E and R132E. In some embodiments, the APOBEC1 domain comprises three mutations of W90Y, R126E and R132E.

In some embodiments, one or more mutations in the cytidine deaminase as disclosed herein reduce the editing window width to about 2 nucleotides. In some embodiments, one or more mutations in the cytidine deaminase as disclosed herein reduce the editing window width to about 1 nucleotide. In some embodiments, one or more mutations in the cytidine deaminase as disclosed herein reduce the editing window width while only minimally or modestly affecting the editing efficiency of the enzyme. In some embodiments, one or more mutations in the cytidine deaminase as disclosed herein reduce the editing window width without reducing the editing efficiency of the enzyme. In some embodiments, one or more mutations in the cytidine deaminase as disclosed herein enable discrimination of neighboring cytidine nucleotides, which would be otherwise edited with similar efficiency by the cytidine deaminase.

In some embodiments, the cytidine deaminase protein further comprises or is connected to one or more double-stranded RNA (dsRNA) binding motifs (dsRBMs) or domains (dsRBDs) for recognizing and binding to double-stranded nucleic acid substrates. In some embodiments, the interaction between the cytidine deaminase and the substrate is mediated by one or more additional protein factor(s), including a CRISPR/CAS protein factor. In some embodiments, the interaction between the cytidine deaminase and the substrate is further mediated by one or more nucleic acid component(s), including a guide RNA.

According to the present invention, the substrate of the cytidine deaminase is an DNA single strand bubble of a RNA duplex comprising a Cytosine of interest, made accessible to the cytidine deaminase upon binding of the guide molecule to its DNA target which then forms the CRISPR-Cas complex with the CRISPR-Cas enzyme, whereby the cytosine deaminase is fused to or is capable of binding to one or more components of the CRISPR-Cas complex, i.e. the CRISPR-Cas enzyme and/or the guide molecule. The particular features of the guide molecule and CRISPR-Cas enzyme are detailed below.

The cytidine deaminase or catalytic domain thereof may be a human, a rat, or a lamprey cytidine deaminase protein or catalytic domain thereof.

The cytidine deaminase protein or catalytic domain thereof may be an apolipoprotein B mRNA-editing complex (APOBEC) family deaminase. The cytidine deaminase protein or catalytic domain thereof may be an activation-induced deaminase (AID). The cytidine deaminase protein or catalytic domain thereof may be a cytidine deaminase 1 (CDA1).

The cytidine deaminase protein or catalytic domain thereof may be an APOBEC1 deaminase. The APOBEC1 deaminase may comprise one or more mutations corresponding to W90A, W90Y, R118A, H121R, H122R, R126A, R126E, or R132E in rat APOBEC1, or an APOBEC3G deaminase comprising one or more mutations corresponding to W285A, W285Y, R313A, D316R, D317R, R320A, R320E, or R326E in human APOBEC3G.

The system may further comprise a uracil glycosylase inhibitor (UGI). Inn some embodiments, the cytidine deaminase protein or catalytic domain thereof is delivered together with a uracil glycosylase inhibitor (UGI). The GI may be linked (e.g., covalently linked) to the cytidine deaminase protein or catalytic domain thereof and/or a catalytically inactive CRISPR-Cas protein.

### Regulation of Post-Translational Modification of Gene Products

In some cases, base editing may be used for regulating post-translational modification of a gene products. In some cases, an amino acid residue that is a post-translational modification site may be mutated by base editing to an amino residue that cannot be modified. Examples of such post-translational modifications include disulfide bond formation, glycosylation, lipidation, acetylation, phosphorylation, methylation, ubiquitination, sumoylation, or any combinations thereof.

### Base Editing Guide Molecule Design Considerations

In some embodiments, the guide sequence is an RNA sequence of between 10 to 50 nt in length, but more particularly of about 20-30 nt advantageously about 20 nt, 23-25 nt or 24 nt. In base editing embodiments, the guide sequence is selected so as to ensure that it hybridizes to the target sequence comprising the adenosine to be deaminated. This is described more in detail below. Selection can encompass further steps which increase efficacy and specificity of deamination.

In some embodiments, the guide sequence is about 20 nt to about 30 nt long and hybridizes to the target DNA strand to form an almost perfectly matched duplex, except for having a dA-C mismatch at the target adenosine site. Particularly, in some embodiments, the dA-C mismatch is located close to the center of the target sequence (and thus the center of the duplex upon hybridization of the guide sequence to the target sequence), thereby restricting the adenosine deaminase to a narrow editing window (e.g., about 4 bp wide). In some embodiments, the target sequence may comprise more than one target adenosine to be deaminated. In further embodiments the target sequence may further comprise one or more dA-C mismatch 3′ to the target adenosine site. In some embodiments, to avoid off-target editing at an unintended Adenine site in the target sequence, the guide sequence can be designed to comprise a non-pairing Guanine at a position corresponding to said unintended Adenine to introduce a dA-G mismatch, which is catalytically unfavorable for certain adenosine deaminases such as ADAR1 and ADAR2. See Wong et al., RNA 7:846-858 (2001), which is incorporated herein by reference in its entirety.

In some embodiments, a CRISPR-Cas guide sequence having a canonical length (e.g., about 20 nt for AacC2c1) is used to form a heteroduplex with the target DNA. In some embodiments, a CRISPR-Cas guide molecule longer than the canonical length (e.g., >20 nt for AacC2c1) is used to form a heteroduplex with the target DNA including outside of the CRISPR-Cas-guide RNA-target DNA complex. This can be of interest where deamination of more than one adenine within a given stretch of nucleotides is of interest. In alternative embodiments, it is of interest to maintain the limitation of the canonical guide sequence length. In some embodiments, the guide sequence is designed to introduce a dA-C mismatch outside of the canonical length of CRISPR-Cas guide, which may decrease steric hindrance by CRISPR-Cas and increase the frequency of contact between the adenosine deaminase and the dA-C mismatch.

In some base editing embodiments, the position of the mismatched nucleobase (e.g., cytidine) is calculated from where the PAM would be on a DNA target. In some embodiments, the mismatched nucleobase is positioned 12-21 nt from the PAM, or 13-21 nt from the PAM, or 14-21 nt from the PAM, or 14-20 nt from the PAM, or 15-20 nt from the PAM, or 16-20 nt from the PAM, or 14-19 nt from the PAM, or 15-19 nt from the PAM, or 16-19 nt from the PAM, or 17-19 nt from the PAM, or about 20 nt from the PAM, or about 19 nt from the PAM, or about 18 nt from the PAM, or about 17 nt from the PAM, or about 16 nt from the PAM, or about 15 nt from the PAM, or about 14 nt from the PAM. In a preferred embodiment, the mismatched nucleobase is positioned 17-19 nt or 18 nt from the PAM.

Mismatch distance is the number of bases between the 3′ end of the CRISPR-Cas spacer and the mismatched nucleobase (e.g., cytidine), wherein the mismatched base is included as part of the mismatch distance calculation. In some embodiment, the mismatch distance is 1-10 nt, or 1-9 nt, or 1-8 nt, or 2-8 nt, or 2-7 nt, or 2-6 nt, or 3-8 nt, or 3-7 nt, or 3-6 nt, or 3-5 nt, or about 2 nt, or about 3 nt, or about 4 nt, or about 5 nt, or about 6 nt, or about 7 nt, or about 8 nt. In a preferred embodiment, the mismatch distance is 3-5 nt or 4 nt.

In some embodiment, the editing window of a CRISPR-Cas-ADAR system described herein is 12-21 nt from the PAM, or 13-21 nt from the PAM, or 14-21 nt from the PAM, or 14-20 nt from the PAM, or 15-20 nt from the PAM, or 16-20 nt from the PAM, or 14-19 nt from the PAM, or 15-19 nt from the PAM, or 16-19 nt from the PAM, or 17-19 nt from the PAM, or about 20 nt from the PAM, or about 19 nt from the PAM, or about 18 nt from the PAM, or about 17 nt from the PAM, or about 16 nt from the PAM, or about 15 nt from the PAM, or about 14 nt from the PAM. In some embodiment, the editing window of the CRISPR-Cas-ADAR system described herein is 1-10 nt from the 3′ end of the CRISPR-Cas spacer, or 1-9 nt from the 3′ end of the CRISPR-Cas spacer, or 1-8 nt from the 3′ end of the CRISPR-Cas spacer, or 2-8 nt from the 3′ end of the Cas spacer, or 2-7 nt from the 3′ end of the CRISPR-Cas spacer, or 2-6 nt from the 3′ end of the CRISPR-Cas spacer, or 3-8 nt from the 3′ end of the CRISPR-Cas spacer, or 3-7 nt from the 3′ end of the CRISPR-Cas spacer, or 3-6 nt from the 3′ end of the CRISPR-Cas spacer, or 3-5 nt from the 3′ end of the CRISPR-Cas spacer, or about 2 nt from the 3′ end of the CRISPR-Cas spacer, or about 3 nt from the 3′ end of the CRISPR-Cas spacer, or about 4 nt from the 3′ end of the CRISPR-Cas spacer, or about 5 nt from the 3′ end of the CRISPR-Cas spacer, or about 6 nt from the 3′ end of the CRISPR-Cas spacer, or about 7 nt from the 3′ end of the CRISPR-Cas spacer, or about 8 nt from the 3′ end of the CRISPR-Cas spacer.

### Linkers

The deaminase herein may be fused to a Cas protein via a linker. It is further envisaged that RNA adenosine methylase (N(6)-methyladenosine) can be fused to the RNA targeting effector proteins of the invention and targeted to a transcript of interest. This methylase causes reversible methylation, has regulatory roles and may affect gene expression and cell fate decisions by modulating multiple RNA-related cellular pathways (Fu et al Nat Rev Genet. 2014; 15(5):293-306).

ADAR or other RNA modification enzymes may be linked (e.g., fused) to CRISPR-Cas or a dead CRISPR-Cas protein via a linker, e.g., to the C terminus or the N-terminus of CRISPR-Cas or dead CRISPR-Cas.

The term “linker” as used in reference to a fusion protein refers to a molecule which joins the proteins to form a fusion protein. Generally, such molecules have no specific biological activity other than to join or to preserve some minimum distance or other spatial relationship between the proteins. However, in certain embodiments, the linker may be selected to influence some property of the linker and/or the fusion protein such as the folding, net charge, or hydrophobicity of the linker.

Suitable linkers for use in the methods of the present invention are well known to those of skill in the art and include, but are not limited to, straight or branched-chain carbon linkers, heterocyclic carbon linkers, or peptide linkers. However, as used herein the linker may also be a covalent bond (carbon-carbon bond or carbon-heteroatom bond). In particular embodiments, the linker is used to separate the CRISPR-Cas protein and the nucleotide deaminase by a distance sufficient to ensure that each protein retains its required functional property. Preferred peptide linker sequences adopt a flexible extended conformation and do not exhibit a propensity for developing an ordered secondary structure. In certain embodiments, the linker can be a chemical moiety which can be monomeric, dimeric, multimeric or polymeric. Preferably, the linker comprises amino acids. Typical amino acids in flexible linkers include Gly, Asn and Ser. Accordingly, in particular embodiments, the linker comprises a combination of one or more of Gly, Asn and Ser amino acids. Other near neutral amino acids, such as Thr and Ala, also may be used in the linker sequence. Exemplary linkers are disclosed in Maratea et al. (1985), Gene 40: 39-46; Murphy et al. (1986) Proc. Nat'l. Acad. Sci. USA 83: 8258-62; U.S. Pat. Nos. 4,935,233; 4,751,180; WO2019126709.

A nucleotide deaminase or other RNA modification enzyme may be linked to CRISPR-Cas or a dead CRISPR-Cas via one or more amino acids. In some cases, the nucleotide deaminase may be linked to the CRISPR-Cas or a dead CRISPR-Cas via one or more amino acids 411-429, 114-124, 197-241, and 607-624. The amino acid position may correspond to a CRISPR-Cas ortholog disclosed herein. In certain examples, the nucleotide deaminase may be is linked to the dead CRISPR-Cas via one or more amino acids corresponding to amino 411-429, 114-124, 197-241, and 607-624 of Prevotella buccae CRISPR-Cas.

### Guide Molecules

As used herein, the term “guide sequence” and “guide molecule” in the context of a CRISPR-Cas system, comprises any polynucleotide sequence having sufficient complementarity with a target nucleic acid sequence to hybridize with the target nucleic acid sequence and direct sequence-specific binding of a nucleic acid-targeting complex to the target nucleic acid sequence. The guide sequences made using the methods disclosed herein may be a full-length guide sequence, a truncated guide sequence, a full-length sgRNA sequence, a truncated sgRNA sequence, or an E+F sgRNA sequence. In some embodiments, the degree of complementarity of the guide sequence to a given target sequence, when optimally aligned using a suitable alignment algorithm, is about or more than about 50%, 60%, 75%, 80%, 85%, 90%, 95%, 97.5%, 99%, or more. In certain example embodiments, the guide molecule comprises a guide sequence that may be designed to have at least one mismatch with the target sequence, such that a RNA duplex formed between the guide sequence and the target sequence. Accordingly, the degree of complementarity is preferably less than 99%. For instance, where the guide sequence consists of 24 nucleotides, the degree of complementarity is more particularly about 96% or less. In particular embodiments, the guide sequence is designed to have a stretch of two or more adjacent mismatching nucleotides, such that the degree of complementarity over the entire guide sequence is further reduced. For instance, where the guide sequence consists of 24 nucleotides, the degree of complementarity is more particularly about 96% or less, more particularly, about 92% or less, more particularly about 88% or less, more particularly about 84% or less, more particularly about 80% or less, more particularly about 76% or less, more particularly about 72% or less, depending on whether the stretch of two or more mismatching nucleotides encompasses 2, 3, 4, 5, 6 or 7 nucleotides, etc. In some embodiments, aside from the stretch of one or more mismatching nucleotides, the degree of complementarity, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net). The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay as described herein. Similarly, cleavage of a target nucleic acid sequence (or a sequence in the vicinity thereof) may be evaluated in a test tube by providing the target nucleic acid sequence, components of a nucleic acid-targeting complex, including the guide sequence to be tested and a control guide sequence different from the test guide sequence, and comparing binding or rate of cleavage at or in the vicinity of the target sequence between the test and control guide sequence reactions. Other assays are possible, and will occur to those skilled in the art. A guide sequence, and hence a nucleic acid-targeting guide RNA may be selected to target any target nucleic acid sequence.

In certain embodiments, the guide sequence or spacer length of the guide molecules is from 15 to 50 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27-30 nt, e.g., 27, 28, 29, or 30 nt, from 30-35 nt, e.g., 30, 31, 32, 33, 34, or 35 nt, or 35 nt or longer. In certain example embodiment, the guide sequence is 15, 16, 17, 18, 19, 20, 21, 22, 23, 24, 25, 26, 27, 28, 29, 30, 31, 32, 33, 34, 35, 36, 37, 38, 39 40, 41, 42, 43, 44, 45, 46, 47 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nt.

In some embodiments, the guide sequence is an RNA sequence of between 10 to 50 nt in length, but more particularly of about 20-30 nt advantageously about 20 nt, 23-25 nt or 24 nt. The guide sequence is selected so as to ensure that it hybridizes to the target sequence. This is described more in detail below. Selection can encompass further steps which increase efficacy and specificity.

In some embodiments, the guide sequence has a canonical length (e.g., about 15-30 nt) is used to hybridize with the target RNA or DNA. In some embodiments, a guide molecule is longer than the canonical length (e.g., >30 nt) is used to hybridize with the target RNA or DNA, such that a region of the guide sequence hybridizes with a region of the RNA or DNA strand outside of the Cas-guide target complex. This can be of interest where additional modifications, such deamination of nucleotides is of interest. In alternative embodiments, it is of interest to maintain the limitation of the canonical guide sequence length.

In some embodiments, the sequence of the guide molecule (direct repeat and/or spacer) is selected to reduce the degree secondary structure within the guide molecule. In some embodiments, about or less than about 75%, 50%, 40%, 30%, 25%, 20%, 15%, 10%, 5%, 1%, or fewer of the nucleotides of the nucleic acid-targeting guide RNA participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and GM Church, 2009, Nature Biotechnology 27(12): 1151-62).

In some embodiments, it is of interest to reduce the susceptibility of the guide molecule to RNA cleavage, such as to cleavage by Cas13. Accordingly, in particular embodiments, the guide molecule is adjusted to avoid cleavage by Cas13 or other RNA-cleaving enzymes.

In certain embodiments, the guide molecule comprises non-naturally occurring nucleic acids and/or non-naturally occurring nucleotides and/or nucleotide analogs, and/or chemically modifications. Preferably, these non-naturally occurring nucleic acids and non-naturally occurring nucleotides are located outside the guide sequence. Non-naturally occurring nucleic acids can include, for example, mixtures of naturally and non-naturally occurring nucleotides. Non-naturally occurring nucleotides and/or nucleotide analogs may be modified at the ribose, phosphate, and/or base moiety. In an embodiment of the invention, a guide nucleic acid comprises ribonucleotides and non-ribonucleotides. In one such embodiment, a guide comprises one or more ribonucleotides and one or more deoxyribonucleotides. In an embodiment of the invention, the guide comprises one or more non-naturally occurring nucleotide or nucleotide analog such as a nucleotide with phosphorothioate linkage, a locked nucleic acid (LNA) nucleotides comprising a methylene bridge between the 2′ and 4′ carbons of the ribose ring, or bridged nucleic acids (BNA). Other examples of modified nucleotides include 2′-O-methyl analogs, 2′-deoxy analogs, or 2′-fluoro analogs. Further examples of modified bases include, but are not limited to, 2-aminopurine, 5-bromo-uridine, pseudouridine, inosine, 7-methylguanosine. Examples of guide RNA chemical modifications include, without limitation, incorporation of 2′-O-methyl (M), 2′-O-methyl 3′ phosphorothioate (MS), S-constrained ethyl(cEt), or 2′-O-methyl 3′ thioPACE (MSP) at one or more terminal nucleotides. Such chemically modified guides can comprise increased stability and increased activity as compared to unmodified guides, though on-target vs. off-target specificity is not predictable. (See, Hendel, 2015, Nat Biotechnol. 33(9):985-9, doi: 10.1038/nbt.3290, published online 29 Jun. 2015 Ragdarm et al., 0215, PNAS, E7110-E7111; Allerson et al., J. Med. Chem. 2005, 48:901-904; Bramsen et al., Front. Genet., 2012, 3:154; Deng et al., PNAS, 2015, 112:11870-11875; Sharma et al., Med Chem Comm., 2014, 5:1454-1471; Hendel et al., Nat. Biotechnol. (2015) 33(9): 985-989; Li et al., Nature Biomedical Engineering, 2017, 1, 0066 DOI:10.1038/s41551-017-0066). In some embodiments, the 5′ and/or 3′ end of a guide RNA is modified by a variety of functional moieties including fluorescent dyes, polyethylene glycol, cholesterol, proteins, or detection tags. (See Kelly et al., 2016, J. Biotech. 233:74-83). In certain embodiments, a guide comprises ribonucleotides in a region that binds to a target RNA and one or more deoxyribonucletides and/or nucleotide analogs in a region that binds to Cas13. In an embodiment of the invention, deoxyribonucleotides and/or nucleotide analogs are incorporated in engineered guide structures, such as, without limitation, stem-loop regions, and the seed region. For Cas13 guide, in certain embodiments, the modification is not in the 5′-handle of the stem-loop regions. Chemical modification in the 5′-handle of the stem-loop region of a guide may abolish its function (see Li, et al., Nature Biomedical Engineering, 2017, 1:0066). In certain embodiments, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or 75 nucleotides of a guide is chemically modified. In some embodiments, 3-5 nucleotides at either the 3′ or the 5′ end of a guide is chemically modified. In some embodiments, only minor modifications are introduced in the seed region, such as 2′-F modifications. In some embodiments, 2′-F modification is introduced at the 3′ end of a guide. In certain embodiments, three to five nucleotides at the 5′ and/or the 3′ end of the guide are chemically modified with 2′-O-methyl (M), 2′-O-methyl 3′ phosphorothioate (MS), S-constrained ethyl(cEt), or 2′-O-methyl 3′ thioPACE (MSP). Such modification can enhance genome editing efficiency (see Hendel et al., Nat. Biotechnol. (2015) 33(9): 985-989). In certain embodiments, all of the phosphodiester bonds of a guide are substituted with phosphorothioates (PS) for enhancing levels of gene disruption. In certain embodiments, more than five nucleotides at the 5′ and/or the 3′ end of the guide are chemically modified with 2′-O-Me, 2′-F or S-constrained ethyl(cEt). Such chemically modified guide can mediate enhanced levels of gene disruption (see Ragdarm et al., 0215, PNAS, E7110-E7111). In an embodiment of the invention, a guide is modified to comprise a chemical moiety at its 3′ and/or 5′ end. Such moieties include, but are not limited to amine, azide, alkyne, thio, dibenzocyclooctyne (DBCO), or Rhodamine. In certain embodiment, the chemical moiety is conjugated to the guide by a linker, such as an alkyl chain. In certain embodiments, the chemical moiety of the modified guide can be used to attach the guide to another molecule, such as DNA, RNA, protein, or nanoparticles. Such chemically modified guide can be used to identify or enrich cells generically edited by a CRISPR system (see Lee et al., eLife, 2017, 6:e25312, DOI:10.7554).

In some embodiments, the modification to the guide is a chemical modification, an insertion, a deletion or a split. In some embodiments, the chemical modification includes, but is not limited to, incorporation of 2′-O-methyl (M) analogs, 2′-deoxy analogs, 2-thiouridine analogs, N6-methyladenosine analogs, 2′-fluoro analogs, 2-aminopurine, 5-bromo-uridine, pseudouridine (Ψ), N1-methylpseudouridine (melΨ), 5-methoxyuridine (5moU), inosine, 7-methylguanosine, 2′-O-methyl 3′phosphorothioate (MS), S-constrained ethyl(cEt), phosphorothioate (PS), or 2′-O-methyl 3′thioPACE (MSP). In some embodiments, the guide comprises one or more of phosphorothioate modifications. In certain embodiments, at least 1, 2, 3, 4, 5, 6, 7, 8, 9, 10, 11, 12, 13, 14, 15, 16, 17, 18, 19, 20, or 25 nucleotides of the guide are chemically modified. In certain embodiments, one or more nucleotides in the seed region are chemically modified. In certain embodiments, one or more nucleotides in the 3′-terminus are chemically modified. In certain embodiments, none of the nucleotides in the 5′-handle is chemically modified. In some embodiments, the chemical modification in the seed region is a minor modification, such as incorporation of a 2′-fluoro analog. In a specific embodiment, one nucleotide of the seed region is replaced with a 2′-fluoro analog. In some embodiments, 5 to 10 nucleotides in the 3′-terminus are chemically modified. Such chemical modifications at the 3′-terminus of the Cas13 CrRNA may improve Cas13 activity. In a specific embodiment, 1, 2, 3, 4, 5, 6, 7, 8, 9 or 10 nucleotides in the 3′-terminus are replaced with 2′-fluoro analogues. In a specific embodiment, 1, 2, 3, 4, 5, 6, 7, 8, 9 or 10 nucleotides in the 3′-terminus are replaced with 2′-O-methyl (M) analogs.

In some embodiments, the loop of the 5′-handle of the guide is modified. In some embodiments, the loop of the 5′-handle of the guide is modified to have a deletion, an insertion, a split, or chemical modifications. In certain embodiments, the modified loop comprises 3, 4, or 5 nucleotides. In certain embodiments, the loop comprises the sequence of UCUU, UUUU, UAUU, or UGUU (SEQ. I.D. Nos. 1-4).

In some embodiments, the guide molecule forms a stemloop with a separate non-covalently linked sequence, which can be DNA or RNA. In particular embodiments, the sequences forming the guide are first synthesized using the standard phosphoramidite synthetic protocol (Herdewijn, P., ed., Methods in Molecular Biology Col 288, Oligonucleotide Synthesis: Methods and Applications, Humana Press, New Jersey (2012)). In some embodiments, these sequences can be functionalized to contain an appropriate functional group for ligation using the standard protocol known in the art (Hermanson, G. T., Bioconjugate Techniques, Academic Press (2013)). Examples of functional groups include, but are not limited to, hydroxyl, amine, carboxylic acid, carboxylic acid halide, carboxylic acid active ester, aldehyde, carbonyl, chlorocarbonyl, imidazolylcarbonyl, hydrozide, semicarbazide, thio semicarbazide, thiol, maleimide, haloalkyl, sufonyl, ally, propargyl, diene, alkyne, and azide. Once this sequence is functionalized, a covalent chemical bond or linkage can be formed between this sequence and the direct repeat sequence. Examples of chemical bonds include, but are not limited to, those based on carbamates, ethers, esters, amides, imines, amidines, aminotrizines, hydrozone, disulfides, thioethers, thioesters, phosphorothioates, phosphorodithioates, sulfonamides, sulfonates, fulfones, sulfoxides, ureas, thioureas, hydrazide, oxime, triazole, photolabile linkages, C—C bond forming groups such as Diels-Alder cyclo-addition pairs or ring-closing metathesis pairs, and Michael reaction pairs.

In some embodiments, these stem-loop forming sequences can be chemically synthesized. In some embodiments, the chemical synthesis uses automated, solid-phase oligonucleotide synthesis machines with 2′-acetoxyethyl orthoester (2′-ACE) (Scaringe et al., J. Am. Chem. Soc. (1998) 120: 11820-11821; Scaringe, Methods Enzymol. (2000) 317: 3-18) or 2′-thionocarbamate (2′-TC) chemistry (Dellinger et al., J. Am. Chem. Soc. (2011) 133: 11540-11546; Hendel et al., Nat. Biotechnol. (2015) 33:985-989).

In certain embodiments, the guide molecule comprises (1) a guide sequence capable of hybridizing to a target locus and (2) a tracr mate or direct repeat sequence whereby the direct repeat sequence is located upstream (i.e., 5′) from the guide sequence. In a particular embodiment the seed sequence (i.e. the sequence essential critical for recognition and/or hybridization to the sequence at the target locus) of th guide sequence is approximately within the first 10 nucleotides of the guide sequence.

In a particular embodiment the guide molecule comprises a guide sequence linked to a direct repeat sequence, wherein the direct repeat sequence comprises one or more stem loops or optimized secondary structures. In particular embodiments, the direct repeat has a minimum length of 16 nts and a single stem loop. In further embodiments the direct repeat has a length longer than 16 nts, preferably more than 17 nts, and has more than one stem loops or optimized secondary structures. In particular embodiments the guide molecule comprises or consists of the guide sequence linked to all or part of the natural direct repeat sequence. A typical Type V or Type VI CRISPR-cas guide molecule comprises (in 3′ to 5′ direction or in 5′ to 3′ direction): a guide sequence a first complimentary stretch (the “repeat”), a loop (which is typically 4 or 5 nucleotides long), a second complimentary stretch (the “anti-repeat” being complimentary to the repeat), and a poly A (often poly U in RNA) tail (terminator). In certain embodiments, the direct repeat sequence retains its natural architecture and forms a single stem loop. In particular embodiments, certain aspects of the guide architecture can be modified, for example by addition, subtraction, or substitution of features, whereas certain other aspects of guide architecture are maintained. Preferred locations for engineered guide molecule modifications, including but not limited to insertions, deletions, and substitutions include guide termini and regions of the guide molecule that are exposed when complexed with the CRISPR-Cas protein and/or target, for example the stemloop of the direct repeat sequence.

In particular embodiments, the stem comprises at least about 4 bp comprising complementary X and Y sequences, although stems of more, e.g., 5, 6, 7, 8, 9, 10, 11 or 12 or fewer, e.g., 3, 2, base pairs are also contemplated. Thus, for example X2-10 and Y2-10 (wherein X and Y represent any complementary set of nucleotides) may be contemplated. In one aspect, the stem made of the X and Y nucleotides, together with the loop will form a complete hairpin in the overall secondary structure; and, this may be advantageous and the amount of base pairs can be any amount that forms a complete hairpin. In one aspect, any complementary X:Y basepairing sequence (e.g., as to length) is tolerated, so long as the secondary structure of the entire guide molecule is preserved. In one aspect, the loop that connects the stem made of X:Y basepairs can be any sequence of the same length (e.g., 4 or 5 nucleotides) or longer that does not interrupt the overall secondary structure of the guide molecule. In one aspect, the stemloop can further comprise, e.g. an MS2 aptamer. In one aspect, the stem comprises about 5-7 bp comprising complementary X and Y sequences, although stems of more or fewer basepairs are also contemplated. In one aspect, non-Watson Crick basepairing is contemplated, where such pairing otherwise generally preserves the architecture of the stemloop at that position.

In particular embodiments the natural hairpin or stemloop structure of the guide molecule is extended or replaced by an extended stemloop. It has been demonstrated that extension of the stem can enhance the assembly of the guide molecule with the CRISPR-Cas protein (Chen et al. Cell. (2013); 155(7): 1479-1491). In particular embodiments the stem of the stemloop is extended by at least 1, 2, 3, 4, 5 or more complementary basepairs (i.e. corresponding to the addition of 2, 4, 6, 8, 10 or more nucleotides in the guide molecule). In particular embodiments these are located at the end of the stem, adjacent to the loop of the stemloop.

In particular embodiments, the susceptibility of the guide molecule to RNases or to decreased expression can be reduced by slight modifications of the sequence of the guide molecule which do not affect its function. For instance, in particular embodiments, premature termination of transcription, such as premature transcription of U6 Pol-III, can be removed by modifying a putative Pol-III terminator (4 consecutive U's) in the guide molecules sequence. Where such sequence modification is required in the stemloop of the guide molecule, it is preferably ensured by a basepair flip.

In a particular embodiment the direct repeat may be modified to comprise one or more protein-binding RNA aptamers. In a particular embodiment, one or more aptamers may be included such as part of optimized secondary structure. Such aptamers may be capable of binding a bacteriophage coat protein as detailed further herein.

In some embodiments, the guide molecule forms a duplex with a target RNA comprising at least one target cytosine residue to be edited. Upon hybridization of the guide RNA molecule to the target RNA, the cytidine deaminase binds to the single strand RNA in the duplex made accessible by the mismatch in the guide sequence and catalyzes deamination of one or more target cytosine residues comprised within the stretch of mismatching nucleotides.

A guide sequence, and hence a nucleic acid-targeting guide RNA may be selected to target any target nucleic acid sequence. The target sequence may be mRNA.

In certain embodiments, the target sequence should be associated with a PAM (protospacer adjacent motif) or PFS (protospacer flanking sequence or site); that is, a short sequence recognized by the CRISPR complex. Depending on the nature of the CRISPR-Cas protein, the target sequence should be selected such that its complementary sequence in the DNA duplex (also referred to herein as the non-target sequence) is upstream or downstream of the PAM. In the embodiments of the present invention where the CRISPR-Cas protein is a Cas13 protein, the complementary sequence of the target sequence is downstream or 3′ of the PAM or upstream or 5′ of the PAM. The precise sequence and length requirements for the PAM differ depending on the Cas13 protein used, but PAMs are typically 2-5 base pair sequences adjacent the protospacer (that is, the target sequence). Examples of the natural PAM sequences for different Cas13 orthologues are provided herein below and the skilled person will be able to identify further PAM sequences for use with a given Cas13 protein.

Further, engineering of the PAM Interacting (PI) domain may allow programming of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10.1038/nature14592. As further detailed herein, the skilled person will understand that Cas13 proteins may be modified analogously.

In particular embodiment, the guide is an escorted guide. By “escorted” is meant that the CRISPR-Cas system or complex or guide is delivered to a selected time or place within a cell, so that activity of the CRISPR-Cas system or complex or guide is spatially or temporally controlled. For example, the activity and destination of the 3 CRISPR-Cas system or complex or guide may be controlled by an escort RNA aptamer sequence that has binding affinity for an aptamer ligand, such as a cell surface protein or other localized cellular component. Alternatively, the escort aptamer may for example be responsive to an aptamer effector on or in the cell, such as a transient effector, such as an external energy source that is applied to the cell at a particular time.

The escorted CRISPR-Cas systems or complexes have a guide molecule with a functional structure designed to improve guide molecule structure, architecture, stability, genetic expression, or any combination thereof. Such a structure can include an aptamer.

Aptamers are biomolecules that can be designed or selected to bind tightly to other ligands, for example using a technique called systematic evolution of ligands by exponential enrichment (SELEX; Tuerk C, Gold L: “Systematic evolution of ligands by exponential enrichment: RNA ligands to bacteriophage T4 DNA polymerase.” Science 1990, 249:505-510). Nucleic acid aptamers can for example be selected from pools of random-sequence oligonucleotides, with high binding affinities and specificities for a wide range of biomedically relevant targets, suggesting a wide range of therapeutic utilities for aptamers (Keefe, Anthony D., Supriya Pai, and Andrew Ellington. “Aptamers as therapeutics.” Nature Reviews Drug Discovery 9.7 (2010): 537-550). These characteristics also suggest a wide range of uses for aptamers as drug delivery vehicles (Levy-Nissenbaum, Etgar, et al. “Nanotechnology and aptamers: applications in drug delivery.” Trends in biotechnology 26.8 (2008): 442-449; and, Hicke B J, Stephens A W. “Escort aptamers: a delivery service for diagnosis and therapy.” J Clin Invest 2000, 106:923-928.). Aptamers may also be constructed that function as molecular switches, responding to a que by changing properties, such as RNA aptamers that bind fluorophores to mimic the activity of green fluorescent protein (Paige, Jeremy S., Karen Y. Wu, and Samie R. Jaffrey. “RNA mimics of green fluorescent protein.” Science 333.6042 (2011): 642-646). It has also been suggested that aptamers may be used as components of targeted siRNA therapeutic delivery systems, for example targeting cell surface proteins (Zhou, Jiehua, and John J. Rossi. “Aptamer-targeted cell-specific RNA interference.” Silence 1.1 (2010): 4).

Accordingly, in particular embodiments, the guide molecule is modified, e.g., by one or more aptamer(s) designed to improve guide molecule delivery, including delivery across the cellular membrane, to intracellular compartments, or into the nucleus. Such a structure can include, either in addition to the one or more aptamer(s) or without such one or more aptamer(s), moiety(ies) so as to render the guide molecule deliverable, inducible or responsive to a selected effector. The invention accordingly comprehends an guide molecule that responds to normal or pathological physiological conditions, including without limitation pH, hypoxia, O2 concentration, temperature, protein concentration, enzymatic concentration, lipid structure, light exposure, mechanical disruption (e.g. ultrasound waves), magnetic fields, electric fields, or electromagnetic radiation.

Light responsiveness of an inducible system may be achieved via the activation and binding of cryptochrome-2 and CIB1. Blue light stimulation induces an activating conformational change in cryptochrome-2, resulting in recruitment of its binding partner CIB1. This binding is fast and reversible, achieving saturation in <15 sec following pulsed stimulation and returning to baseline <15 min after the end of stimulation. These rapid binding kinetics result in a system temporally bound only by the speed of transcription/translation and transcript/protein degradation, rather than uptake and clearance of inducing agents. Cryptochrome-2 activation is also highly sensitive, allowing for the use of low light intensity stimulation and mitigating the risks of phototoxicity. Further, in a context such as the intact mammalian brain, variable light intensity may be used to control the size of a stimulated region, allowing for greater precision than vector delivery alone may offer.

The invention contemplates energy sources such as electromagnetic radiation, sound energy or thermal energy to induce the guide. Advantageously, the electromagnetic radiation is a component of visible light. In a preferred embodiment, the light is a blue light with a wavelength of about 450 to about 495 nm. In an especially preferred embodiment, the wavelength is about 488 nm. In another preferred embodiment, the light stimulation is via pulses. The light power may range from about 0-9 mW/cm2. In a preferred embodiment, a stimulation paradigm of as low as 0.25 sec every 15 sec should result in maximal activation.

The chemical or energy sensitive guide may undergo a conformational change upon induction by the binding of a chemical source or by the energy allowing it act as a guide and have the Cas13 CRISPR-Cas system or complex function. The invention can involve applying the chemical source or energy so as to have the guide function and the Cas13 CRISPR-Cas system or complex function; and optionally further determining that the expression of the genomic locus is altered.

There are several different designs of this chemical inducible system: 1. ABI-PYL based system inducible by Abscisic Acid (ABA) (see, e.g., stke.sciencemag.org/cgi/content/abstract/sigtrans; 4/164/rs2), 2. FKBP-FRB based system inducible by rapamycin (or related chemicals based on rapamycin) (see, e.g., www.nature.com/nmeth/journal/v2/n6/full/nmeth763.html), 3. GID1-GAI based system inducible by Gibberellin (GA) (see, e.g., www.nature.com/nchembio/journal/v8/n5/full/nchembio.922.html).

A chemical inducible system can be an estrogen receptor (ER) based system inducible by 4-hydroxytamoxifen (4OHT) (see, e.g., www.pnas.org/content/104/3/1027.abstract). A mutated ligand-binding domain of the estrogen receptor called ERT2 translocates into the nucleus of cells upon binding of 4-hydroxytamoxifen. In further embodiments of the invention any naturally occurring or engineered derivative of any nuclear receptor, thyroid hormone receptor, retinoic acid receptor, estrogen receptor, estrogen-related receptor, glucocorticoid receptor, progesterone receptor, androgen receptor may be used in inducible systems analogous to the ER based inducible system.

Another inducible system is based on the design using Transient receptor potential (TRP) ion channel based system inducible by energy, heat or radio-wave (see, e.g., www.sciencemag.org/content/336/6081/604). These TRP family proteins respond to different stimuli, including light and heat. When this protein is activated by light or heat, the ion channel will open and allow the entering of ions such as calcium into the plasma membrane. This influx of ions will bind to intracellular ion interacting partners linked to a polypeptide including the guide and the other components of the Cas13 CRISPR-Cas complex or system, and the binding will induce the change of sub-cellular localization of the polypeptide, leading to the entire polypeptide entering the nucleus of cells. Once inside the nucleus, the guide protein and the other components of the Cas13 CRISPR-Cas complex will be active and modulating target gene expression in cells.

While light activation may be an advantageous embodiment, sometimes it may be disadvantageous especially for in vivo applications in which the light may not penetrate the skin or other organs. In this instance, other methods of energy activation are contemplated, in particular, electric field energy and/or ultrasound which have a similar effect.

Electric field energy is preferably administered substantially as described in the art, using one or more electric pulses of from about 1 Volt/cm to about 10 kVolts/cm under in vivo conditions. Instead of or in addition to the pulses, the electric field may be delivered in a continuous manner. The electric pulse may be applied for between 1 s and 500 milliseconds, preferably between 1 s and 100 milliseconds. The electric field may be applied continuously or in a pulsed manner for 5 about minutes.

As used herein, ‘electric field energy’ is the electrical energy to which a cell is exposed. Preferably the electric field has a strength of from about 1 Volt/cm to about 10 kVolts/cm or more under in vivo conditions (see WO97/49450).

As used herein, the term “electric field” includes one or more pulses at variable capacitance and voltage and including exponential and/or square wave and/or modulated wave and/or modulated square wave forms. References to electric fields and electricity should be taken to include reference the presence of an electric potential difference in the environment of a cell. Such an environment may be set up by way of static electricity, alternating current (AC), direct current (DC), etc, as known in the art. The electric field may be uniform, non-uniform or otherwise, and may vary in strength and/or direction in a time dependent manner.

Single or multiple applications of electric field, as well as single or multiple applications of ultrasound are also possible, in any order and in any combination. The ultrasound and/or the electric field may be delivered as single or multiple continuous applications, or as pulses (pulsatile delivery).

Electroporation has been used in both in vitro and in vivo procedures to introduce foreign material into living cells. With in vitro applications, a sample of live cells is first mixed with the agent of interest and placed between electrodes such as parallel plates. Then, the electrodes apply an electrical field to the cell/implant mixture. Examples of systems that perform in vitro electroporation include the Electro Cell Manipulator ECM600 product, and the Electro Square Porator T820, both made by the BTX Division of Genetronics, Inc (see U.S. Pat. No. 5,869,326).

The known electroporation techniques (both in vitro and in vivo) function by applying a brief high voltage pulse to electrodes positioned around the treatment region. The electric field generated between the electrodes causes the cell membranes to temporarily become porous, whereupon molecules of the agent of interest enter the cells. In known electroporation applications, this electric field comprises a single square wave pulse on the order of 1000 V/cm, of about 100 .mu.s duration. Such a pulse may be generated, for example, in known applications of the Electro Square Porator T820.

Preferably, the electric field has a strength of from about 1 V/cm to about 10 kV/cm under in vitro conditions. Thus, the electric field may have a strength of 1 V/cm, 2 V/cm, 3 V/cm, 4 V/cm, 5 V/cm, 6 V/cm, 7 V/cm, 8 V/cm, 9 V/cm, 10 V/cm, 20 V/cm, 50 V/cm, 100 V/cm, 200 V/cm, 300 V/cm, 400 V/cm, 500 V/cm, 600 V/cm, 700 V/cm, 800 V/cm, 900 V/cm, 1 kV/cm, 2 kV/cm, 5 kV/cm, 10 kV/cm, 20 kV/cm, 50 kV/cm or more. More preferably from about 0.5 kV/cm to about 4.0 kV/cm under in vitro conditions. Preferably the electric field has a strength of from about 1 V/cm to about 10 kV/cm under in vivo conditions. However, the electric field strengths may be lowered where the number of pulses delivered to the target site are increased. Thus, pulsatile delivery of electric fields at lower field strengths is envisaged.

Preferably the application of the electric field is in the form of multiple pulses such as double pulses of the same strength and capacitance or sequential pulses of varying strength and/or capacitance. As used herein, the term “pulse” includes one or more electric pulses at variable capacitance and voltage and including exponential and/or square wave and/or modulated wave/square wave forms.

Preferably the electric pulse is delivered as a waveform selected from an exponential wave form, a square wave form, a modulated wave form and a modulated square wave form.

A preferred embodiment employs direct current at low voltage. Thus, Applicants disclose the use of an electric field which is applied to the cell, tissue or tissue mass at a field strength of between 1V/cm and 20V/cm, for a period of 100 milliseconds or more, preferably 15 minutes or more.

Ultrasound is advantageously administered at a power level of from about 0.05 W/cm2 to about 100 W/cm2. Diagnostic or therapeutic ultrasound may be used, or combinations thereof.

As used herein, the term “ultrasound” refers to a form of energy which consists of mechanical vibrations the frequencies of which are so high they are above the range of human hearing. Lower frequency limit of the ultrasonic spectrum may generally be taken as about 20 kHz. Most diagnostic applications of ultrasound employ frequencies in the range 1 and 15 MHz’ (From Ultrasonics in Clinical Diagnosis, P. N. T. Wells, ed., 2nd. Edition, Publ. Churchill Livingstone [Edinburgh, London & NY, 1977]).

Ultrasound has been used in both diagnostic and therapeutic applications. When used as a diagnostic tool (“diagnostic ultrasound”), ultrasound is typically used in an energy density range of up to about 100 mW/cm2 (FDA recommendation), although energy densities of up to 750 mW/cm2 have been used. In physiotherapy, ultrasound is typically used as an energy source in a range up to about 3 to 4 W/cm2 (WHO recommendation). In other therapeutic applications, higher intensities of ultrasound may be employed, for example, HIFU at 100 W/cm up to 1 kW/cm2 (or even higher) for short periods of time. The term “ultrasound” as used in this specification is intended to encompass diagnostic, therapeutic and focused ultrasound.

Focused ultrasound (FUS) allows thermal energy to be delivered without an invasive probe (see Morocz et al 1998 Journal of Magnetic Resonance Imaging Vol. 8, No. 1, pp. 136-142. Another form of focused ultrasound is high intensity focused ultrasound (HIFU) which is reviewed by Moussatov et al in Ultrasonics (1998) Vol. 36, No. 8, pp.893-900 and TranHuuHue et al in Acustica (1997) Vol. 83, No. 6, pp. 1103-1106.

Preferably, a combination of diagnostic ultrasound and a therapeutic ultrasound is employed. This combination is not intended to be limiting, however, and the skilled reader will appreciate that any variety of combinations of ultrasound may be used. Additionally, the energy density, frequency of ultrasound, and period of exposure may be varied.

Preferably the exposure to an ultrasound energy source is at a power density of from about 0.05 to about 100 Wcm-². Even more preferably, the exposure to an ultrasound energy source is at a power density of from about 1 to about 15 Wcm-².

Preferably the exposure to an ultrasound energy source is at a frequency of from about 0.015 to about 10.0 MHz. More preferably the exposure to an ultrasound energy source is at a frequency of from about 0.02 to about 5.0 MHz or about 6.0 MHz. Most preferably, the ultrasound is applied at a frequency of 3 MHz.

Preferably the exposure is for periods of from about 10 milliseconds to about 60 minutes. Preferably the exposure is for periods of from about 1 second to about 5 minutes. More preferably, the ultrasound is applied for about 2 minutes. Depending on the particular target cell to be disrupted, however, the exposure may be for a longer duration, for example, for 15 minutes.

Advantageously, the target tissue is exposed to an ultrasound energy source at an acoustic power density of from about 0.05 Wcm-2 to about 10 Wcm-2 with a frequency ranging from about 0.015 to about 10 MHz (see WO 98/52609). However, alternatives are also possible, for example, exposure to an ultrasound energy source at an acoustic power density of above 100 Wcm-2, but for reduced periods of time, for example, 1000 Wcm-2 for periods in the millisecond range or less.

Preferably the application of the ultrasound is in the form of multiple pulses; thus, both continuous wave and pulsed wave (pulsatile delivery of ultrasound) may be employed in any combination. For example, continuous wave ultrasound may be applied, followed by pulsed wave ultrasound, or vice versa. This may be repeated any number of times, in any order and combination. The pulsed wave ultrasound may be applied against a background of continuous wave ultrasound, and any number of pulses may be used in any number of groups.

Preferably, the ultrasound may comprise pulsed wave ultrasound. In a highly preferred embodiment, the ultrasound is applied at a power density of 0.7 Wcm-2 or 1.25 Wcm-2 as a continuous wave. Higher power densities may be employed if pulsed wave ultrasound is used.

Use of ultrasound is advantageous as, like light, it may be focused accurately on a target. Moreover, ultrasound is advantageous as it may be focused more deeply into tissues unlike light. It is therefore better suited to whole-tissue penetration (such as but not limited to a lobe of the liver) or whole organ (such as but not limited to the entire liver or an entire muscle, such as the heart) therapy. Another important advantage is that ultrasound is a non-invasive stimulus which is used in a wide variety of diagnostic and therapeutic applications. By way of example, ultrasound is well known in medical imaging techniques and, additionally, in orthopedic therapy. Furthermore, instruments suitable for the application of ultrasound to a subject vertebrate are widely available and their use is well known in the art.

In particular embodiments, the guide molecule is modified by a secondary structure to increase the specificity of the CRISPR-Cas system and the secondary structure can protect against exonuclease activity and allow for 5′ additions to the guide sequence also referred to herein as a protected guide molecule.

In one aspect, the invention provides for hybridizing a “protector RNA” to a sequence of the guide molecule, wherein the “protector RNA” is an RNA strand complementary to the 3′ end of the guide molecule to thereby generate a partially double-stranded guide RNA. In an embodiment of the invention, protecting mismatched bases (i.e. the bases of the guide molecule which do not form part of the guide sequence) with a perfectly complementary protector sequence decreases the likelihood of target RNA binding to the mismatched basepairs at the 3′ end. In particular embodiments of the invention, additional sequences comprising an extended length may also be present within the guide molecule such that the guide comprises a protector sequence within the guide molecule. This “protector sequence” ensures that the guide molecule comprises a “protected sequence” in addition to an “exposed sequence” (comprising the part of the guide sequence hybridizing to the target sequence). In particular embodiments, the guide molecule is modified by the presence of the protector guide to comprise a secondary structure such as a hairpin. Advantageously there are three or four to thirty or more, e.g., about 10 or more, contiguous base pairs having complementarity to the protected sequence, the guide sequence or both. It is advantageous that the protected portion does not impede thermodynamics of the CRISPR-Cas system interacting with its target. By providing such an extension including a partially double stranded guide molecule, the guide molecule is considered protected and results in improved specific binding of the CRISPR-Cas complex, while maintaining specific activity.

In particular embodiments, use is made of a truncated guide (tru-guide), i.e. a guide molecule which comprises a guide sequence which is truncated in length with respect to the canonical guide sequence length. As described by Nowak et al. (Nucleic Acids Res (2016) 44 (20): 9555-9564), such guides may allow catalytically active CRISPR-Cas enzyme to bind its target without cleaving the target RNA. In particular embodiments, a truncated guide is used which allows the binding of the target but retains only nickase activity of the CRISPR-Cas enzyme.

The present invention may be further illustrated and extended based on aspects of CRISPR-Cas development and use as set forth in the following articles and particularly as relates to delivery of a CRISPR protein complex and uses of an RNA guided endonuclease in cells and organisms:
Multiplex genome engineering using CRISPR-Cas systems. Cong, L., Ran, F. A., Cox, D., Lin, S., Barretto, R., Habib, N., Hsu, P. D., Wu, X., Jiang, W., Marraffini, L. A., & Zhang, F. Science February 15; 339(6121):819-23 (2013);
RNA-guided editing of bacterial genomes using CRISPR-Cas systems. Jiang W., Bikard D., Cox D., Zhang F, Marraffini L A. Nat Biotechnol March; 31(3):233-9 (2013);
One-Step Generation of Mice Carrying Mutations in Multiple Genes by CRISPR-Cas-Mediated Genome Engineering. Wang H., Yang H., Shivalila C S., Dawlaty M M., Cheng A W., Zhang F., Jaenisch R. Cell May 9; 153(4):910-8 (2013);
Optical control of mammalian endogenous transcription and epigenetic states. Konermann S, Brigham M D, Trevino A E, Hsu P D, Heidenreich M, Cong L, Platt R J, Scott D A, Church G M, Zhang F. Nature. August 22; 500(7463):472-6. doi: 10.1038/Nature12466. Epub 2013 Aug. 23 (2013);
Double Nicking by RNA-Guided CRISPR Cas9 for Enhanced Genome Editing Specificity. Ran, F A., Hsu, P D., Lin, C Y., Gootenberg, J S., Konermann, S., Trevino, A E., Scott, D A., Inoue, A., Matoba, S., Zhang, Y., & Zhang, F. Cell August 28. pii: S0092-8674(13)01015-5 (2013-A);
DNA targeting specificity of RNA-guided Cas9 nucleases. Hsu, P., Scott, D., Weinstein, J., Ran, F A., Konermann, S., Agarwala, V., Li, Y., Fine, E., Wu, X., Shalem, O., Cradick, T J., Marraffini, L A., Bao, G., & Zhang, F. Nat Biotechnol doi:10.1038/nbt.2647 (2013);
Genome engineering using the CRISPR-Cas9 system. Ran, F A., Hsu, P D., Wright, J., Agarwala, V., Scott, D A., Zhang, F. Nature Protocols November; 8(11):2281-308 (2013-B);
Genome-Scale CRISPR-Cas9 Knockout Screening in Human Cells. Shalem, O., Sanjana, N E., Hartenian, E., Shi, X., Scott, D A., Mikkelson, T., Heckl, D., Ebert, B L., Root, D E., Doench, J G., Zhang, F. Science December 12. (2013);
Crystal structure of cas9 in complex with guide RNA and target DNA. Nishimasu, H., Ran, F A., Hsu, P D., Konermann, S., Shehata, S I., Dohmae, N., Ishitani, R., Zhang, F., Nureki, O. Cell February 27, 156(5):935-49 (2014);
Genome-wide binding of the CRISPR endonuclease Cas9 in mammalian cells. Wu X., Scott D A., Kriz A J., Chiu A C., Hsu P D., Dadon D B., Cheng A W., Trevino A E., Konermann S., Chen S., Jaenisch R., Zhang F., Sharp P A. Nat Biotechnol. April 20. doi: 10.1038/nbt.2889 (2014);
CRISPR-Cas9 Knockin Mice for Genome Editing and Cancer Modeling. Platt R J, Chen S, Zhou Y, Yim M J, Swiech L, Kempton H R, Dahlman J E, Parnas O, Eisenhaure T M, Jovanovic M, Graham D B, Jhunjhunwala S, Heidenreich M, Xavier R J, Langer R, Anderson D G, Hacohen N, Regev A, Feng G, Sharp P A, Zhang F. Cell 159(2): 440-455 DOI: 10.1016/j.cell.2014.09.014(2014);
Development and Applications of CRISPR-Cas9 for Genome Engineering, Hsu P D, Lander E S, Zhang F., Cell. June 5; 157(6):1262-78 (2014).
Genetic screens in human cells using the CRISPR-Cas9 system, Wang T, Wei J J, Sabatini D M, Lander E S., Science. January 3; 343(6166): 80-84. doi:10.1126/science.1246981 (2014);
Rational design of highly active sgRNAs for CRISPR-Cas9-mediated gene inactivation, Doench J G, Hartenian E, Graham D B, Tothova Z, Hegde M, Smith I, Sullender M, Ebert B L, Xavier R J, Root D E., (published online 3 Sep. 2014) Nat Biotechnol. December; 32(12):1262-7 (2014);
In vivo interrogation of gene function in the mammalian brain using CRISPR-Cas9, Swiech L, Heidenreich M, Banerjee A, Habib N, Li Y, Trombetta J, Sur M, Zhang F., (published online 19 Oct. 2014) Nat Biotechnol. January; 33(1):102-6 (2015);
Genome-scale transcriptional activation by an engineered CRISPR-Cas9 complex, Konermann S, Brigham M D, Trevino A E, Joung J, Abudayyeh O O, Barcena C, Hsu P D, Habib N, Gootenberg J S, Nishimasu H, Nureki O, Zhang F., Nature. January 29; 517(7536):583-8 (2015).
A split-Cas9 architecture for inducible genome editing and transcription modulation, Zetsche B, Volz S E, Zhang F., (published online 2 Feb. 2015) Nat Biotechnol. February; 33(2):139-42 (2015);
Genome-wide CRISPR Screen in a Mouse Model of Tumor Growth and Metastasis, Chen S, Sanjana N E, Zheng K, Shalem O, Lee K, Shi X, Scott D A, Song J, Pan J Q, Weissleder R, Lee H, Zhang F, Sharp P A. Cell 160, 1246-1260, Mar. 12, 2015 (multiplex screen in mouse), and
In vivo genome editing using Staphylococcus aureus Cas9, Ran F A, Cong L, Yan W X, Scott D A, Gootenberg J S, Kriz A J, Zetsche B, Shalem O, Wu X, Makarova K S, Koonin E V, Sharp P A, Zhang F., (published online 1 Apr. 2015), Nature. April 9; 520(7546):186-91 (2015).
Shalem et al., “High-throughput functional genomics using CRISPR-Cas9,” Nature Reviews Genetics 16, 299-311 (May 2015).
Xu et al., “Sequence determinants of improved CRISPR sgRNA design,” Genome Research 25, 1147-1157 (August 2015).
Parnas et al., “A Genome-wide CRISPR Screen in Primary Immune Cells to Dissect Regulatory Networks,” Cell 162, 675-686 (Jul. 30, 2015).
Ramanan et al., CRISPR-Cas9 cleavage of viral DNA efficiently suppresses hepatitis B virus,” Scientific Reports 5:10833. doi: 10.1038/srep10833 (Jun. 2, 2015)
Nishimasu et al., Crystal Structure of Staphylococcus aureus Cas9,” Cell 162, 1113-1126 (Aug. 27, 2015)
BCL11A enhancer dissection by Cas9-mediated in situ saturating mutagenesis, Canver et al., Nature 527(7577):192-7 (Nov. 12, 2015) doi: 10.1038/nature15521. Epub 2015 Sep. 16.
Cpf1 Is a Single RNA-Guided Endonuclease of a Class 2 CRISPR-Cas System, Zetsche et al., Cell 163, 759-71 (Sep. 25, 2015).
Discovery and Functional Characterization of Diverse Class 2 CRISPR-Cas Systems, Shmakov et al., Molecular Cell, 60(3), 385-397 doi: 10.1016/j.molcel.2015.10.008 Epub Oct. 22, 2015.
Rationally engineered Cas9 nucleases with improved specificity, Slaymaker et al., Science 2016 Jan. 1 351(6268): 84-88 doi: 10.1126/science.aad5227. Epub 2015 Dec. 1.
Gao et al, “Engineered Cpf1 Enzymes with Altered PAM Specificities,” bioRxiv 091611; doi: dx.doi.org/10.1101/091611 (Dec. 4, 2016).
   each of which is incorporated herein by reference, may be considered in the practice of the instant invention, and discussed briefly below:
   Cong et al. engineered type II CRISPR-Cas systems for use in eukaryotic cells based on both Streptococcus thermophilus Cas9 and also Streptococcus pyogenes Cas9 and demonstrated that Cas9 nucleases can be directed by short RNAs to induce precise cleavage of DNA in human and mouse cells. Their study further showed that Cas9 as converted into a nicking enzyme can be used to facilitate homology-directed repair in eukaryotic cells with minimal mutagenic activity. Additionally, their study demonstrated that multiple guide sequences can be encoded into a single CRISPR array to enable simultaneous editing of several at endogenous genomic loci sites within the mammalian genome, demonstrating easy programmability and wide applicability of the RNA-guided nuclease technology. This ability to use RNA to program sequence specific DNA cleavage in cells defined a new class of genome engineering tools. These studies further showed that other CRISPR loci are likely to be transplantable into mammalian cells and can also mediate mammalian genome cleavage. Importantly, it can be envisaged that several aspects of the CRISPR-Cas system can be further improved to increase its efficiency and versatility.
   Jiang et al. used the clustered, regularly interspaced, short palindromic repeats (CRISPR)-associated Cas9 endonuclease complexed with dual-RNAs to introduce precise mutations in the genomes of Streptococcus pneumoniae and Escherichia coli. The approach relied on dual-RNA:Cas9-directed cleavage at the targeted genomic site to kill unmutated cells and circumvents the need for selectable markers or counter-selection systems. The study reported reprogramming dual-RNA:Cas9 specificity by changing the sequence of short CRISPR RNA (crRNA) to make single- and multinucleotide changes carried on editing templates. The study showed that simultaneous use of two crRNAs enabled multiplex mutagenesis. Furthermore, when the approach was used in combination with recombineering, in S. pneumoniae, nearly 100% of cells that were recovered using the described approach contained the desired mutation, and in E. coli, 65% that were recovered contained the mutation.
   Wang et al. (2013) used the CRISPR-Cas system for the one-step generation of mice carrying mutations in multiple genes which were traditionally generated in multiple steps by sequential recombination in embryonic stem cells and/or time-consuming intercrossing of mice with a single mutation. The CRISPR-Cas system will greatly accelerate the in vivo study of functionally redundant genes and of epistatic gene interactions.
   Konermann et al. (2013) addressed the need in the art for versatile and robust technologies that enable optical and chemical modulation of DNA-binding domains based CRISPR Cas9 enzyme and also Transcriptional Activator Like Effectors
   Ran et al. (2013-A) described an approach that combined a Cas9 nickase mutant with paired guide RNAs to introduce targeted double-strand breaks. This addresses the issue of the Cas9 nuclease from the microbial CRISPR-Cas system being targeted to specific genomic loci by a guide sequence, which can tolerate certain mismatches to the DNA target and thereby promote undesired off-target mutagenesis. Because individual nicks in the genome are repaired with high fidelity, simultaneous nicking via appropriately offset guide RNAs is required for double-stranded breaks and extends the number of specifically recognized bases for target cleavage. The authors demonstrated that using paired nicking can reduce off-target activity by 50- to 1,500-fold in cell lines and to facilitate gene knockout in mouse zygotes without sacrificing on-target cleavage efficiency. This versatile strategy enables a wide variety of genome editing applications that require high specificity.
   Hsu et al. (2013) characterized SpCas9 targeting specificity in human cells to inform the selection of target sites and avoid off-target effects. The study evaluated >700 guide RNA variants and SpCas9-induced indel mutation levels at >100 predicted genomic off-target loci in 293T and 293FT cells. The authors that SpCas9 tolerates mismatches between guide RNA and target DNA at different positions in a sequence-dependent manner, sensitive to the number, position and distribution of mismatches. The authors further showed that SpCas9-mediated cleavage is unaffected by DNA methylation and that the dosage of SpCas9 and guide RNA can be titrated to minimize off-target modification. Additionally, to facilitate mammalian genome engineering applications, the authors reported providing a web-based software tool to guide the selection and validation of target sequences as well as off-target analyses.
   Ran et al. (2013-B) described a set of tools for Cas9-mediated genome editing via non-homologous end joining (NHEJ) or homology-directed repair (HDR) in mammalian cells, as well as generation of modified cell lines for downstream functional studies. To minimize off-target cleavage, the authors further described a double-nicking strategy using the Cas9 nickase mutant with paired guide RNAs. The protocol provided by the authors experimentally derived guidelines for the selection of target sites, evaluation of cleavage efficiency and analysis of off-target activity. The studies showed that beginning with target design, gene modifications can be achieved within as little as 1-2 weeks, and modified clonal cell lines can be derived within 2-3 weeks.
   Shalem et al. described a new way to interrogate gene function on a genome-wide scale. Their studies showed that delivery of a genome-scale CRISPR-Cas9 knockout (GeCKO) library targeted 18,080 genes with 64,751 unique guide sequences enabled both negative and positive selection screening in human cells. First, the authors showed use of the GeCKO library to identify genes essential for cell viability in cancer and pluripotent stem cells. Next, in a melanoma model, the authors screened for genes whose loss is involved in resistance to vemurafenib, a therapeutic that inhibits mutant protein kinase BRAF. Their studies showed that the highest-ranking candidates included previously validated genes NF1 and MED12 as well as novel hits NF2, CUL3, TADA2B, and TADA1. The authors observed a high level of consistency between independent guide RNAs targeting the same gene and a high rate of hit confirmation, and thus demonstrated the promise of genome-scale screening with Cas9.
   Nishimasu et al. reported the crystal structure of Streptococcus pyogenes Cas9 in complex with sgRNA and its target DNA at 2.5 A° resolution. The structure revealed a bilobed architecture composed of target recognition and nuclease lobes, accommodating the sgRNA:DNA heteroduplex in a positively charged groove at their interface. Whereas the recognition lobe is essential for binding sgRNA and DNA, the nuclease lobe contains the HNH and RuvC nuclease domains, which are properly positioned for cleavage of the complementary and non-complementary strands of the target DNA, respectively. The nuclease lobe also contains a carboxyl-terminal domain responsible for the interaction with the protospacer adjacent motif (PAM). This high-resolution structure and accompanying functional analyses have revealed the molecular mechanism of RNA-guided DNA targeting by Cas9, thus paving the way for the rational design of new, versatile genome-editing technologies.
   Wu et al. mapped genome-wide binding sites of a catalytically inactive Cas9 (dCas9) from Streptococcus pyogenes loaded with single guide RNAs (sgRNAs) in mouse embryonic stem cells (mESCs). The authors showed that each of the four sgRNAs tested targets dCas9 to between tens and thousands of genomic sites, frequently characterized by a 5-nucleotide seed region in the sgRNA and an NGG protospacer adjacent motif (PAM). Chromatin inaccessibility decreases dCas9 binding to other sites with matching seed sequences; thus 70% of off-target sites are associated with genes. The authors showed that targeted sequencing of 295 dCas9 binding sites in mESCs transfected with catalytically active Cas9 identified only one site mutated above background levels. The authors proposed a two-state model for Cas9 binding and cleavage, in which a seed match triggers binding but extensive pairing with target DNA is required for cleavage.
   Platt et al. established a Cre-dependent Cas9 knockin mouse. The authors demonstrated in vivo as well as ex vivo genome editing using adeno-associated virus (AAV)-, lentivirus-, or particle-mediated delivery of guide RNA in neurons, immune cells, and endothelial cells.
   Hsu et al. (2014) is a review article that discusses generally CRISPR-Cas9 history from yogurt to genome editing, including genetic screening of cells.
   Wang et al. (2014) relates to a pooled, loss-of-function genetic screening approach suitable for both positive and negative selection that uses a genome-scale lentiviral single guide RNA (sgRNA) library.
   Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.
   Swiech et al. demonstrate that AAV-mediated SpCas9 genome editing can enable reverse genetic studies of gene function in the brain.
   Konermann et al. (2015) discusses the ability to attach multiple effector domains, e.g., transcriptional activator, functional and epigenomic regulators at appropriate positions on the guide such as stem or tetraloop with and without linkers.
   Zetsche et al. demonstrates that the Cas9 enzyme can be split into two and hence the assembly of Cas9 for activation can be controlled.
   Chen et al. relates to multiplex screening by demonstrating that a genome-wide in vivo CRISPR-Cas9 screen in mice reveals genes regulating lung metastasis.
   Ran et al. (2015) relates to SaCas9 and its ability to edit genomes and demonstrates that one cannot extrapolate from biochemical assays.
   Shalem et al. (2015) described ways in which catalytically inactive Cas9 (dCas9) fusions are used to synthetically repress (CRISPRi) or activate (CRISPRa) expression, showing. advances using Cas9 for genome-scale screens, including arrayed and pooled screens, knockout approaches that inactivate genomic loci and strategies that modulate transcriptional activity.
   Xu et al. (2015) assessed the DNA sequence features that contribute to single guide RNA (sgRNA) efficiency in CRISPR-based screens. The authors explored efficiency of CRISPR-Cas9 knockout and nucleotide preference at the cleavage site. The authors also found that the sequence preference for CRISPRi/a is substantially different from that for CRISPR-Cas9 knockout.
   Parnas et al. (2015) introduced genome-wide pooled CRISPR-Cas9 libraries into dendritic cells (DCs) to identify genes that control the induction of tumor necrosis factor (Tnf) by bacterial lipopolysaccharide (LPS). Known regulators of Tlr4 signaling and previously unknown candidates were identified and classified into three functional modules with distinct effects on the canonical responses to LPS.
   Ramanan et al (2015) demonstrated cleavage of viral episomal DNA (cccDNA) in infected cells. The HBV genome exists in the nuclei of infected hepatocytes as a 3.2 kb double-stranded episomal DNA species called covalently closed circular DNA (cccDNA), which is a key component in the HBV life cycle whose replication is not inhibited by current therapies. The authors showed that sgRNAs specifically targeting highly conserved regions of HBV robustly suppresses viral replication and depleted cccDNA.
   Nishimasu et al. (2015) reported the crystal structures of SaCas9 in complex with a single guide RNA (sgRNA) and its double-stranded DNA targets, containing the 5′-TTGAAT-3′ PAM and the 5′-TTGGGT-3′ PAM. A structural comparison of SaCas9 with SpCas9 highlighted both structural conservation and divergence, explaining their distinct PAM specificities and orthologous sgRNA recognition.
   Canver et al. (2015) demonstrated a CRISPR-Cas9-based functional investigation of non-coding genomic elements. The authors developed pooled CRISPR-Cas9 guide RNA libraries to perform in situ saturating mutagenesis of the human and mouse BCL11A enhancers which revealed critical features of the enhancers.
   Zetsche et al. (2015) reported characterization of Cpf1, a class 2 CRISPR nuclease from Francisella novicida U112 having features distinct from Cas9. Cpf1 is a single RNA-guided endonuclease lacking tracrRNA, utilizes a T-rich protospacer-adjacent motif, and cleaves DNA via a staggered DNA double-stranded break.
   Shmakov et al. (2015) reported three distinct Class 2 CRISPR-Cas systems. Two system CRISPR enzymes (C2c1 and C2c3) contain RuvC-like endonuclease domains distantly related to Cpf1. Unlike Cpf1, C2c1 depends on both crRNA and tracrRNA for DNA cleavage. The third enzyme (C2c2) contains two predicted HEPN RNase domains and is tracrRNA independent.
   Slaymaker et al (2016) reported the use of structure-guided protein engineering to improve the specificity of Streptococcus pyogenes Cas9 (SpCas9). The authors developed “enhanced specificity” SpCas9 (eSpCas9) variants which maintained robust on-target cleavage with reduced off-target effects.

The methods and tools provided herein are may be designed for use with or Cas13, a type II nuclease that does not make use of tracrRNA. Orthologs of Cas13 have been identified in different bacterial species as described herein. Further type II nucleases with similar properties can be identified using methods described in the art (Shmakov et al. 2015, 60:385-397; Abudayeh et al. 2016, Science, 5; 353(6299)). In particular embodiments, such methods for identifying novel CRISPR effector proteins may comprise the steps of selecting sequences from the database encoding a seed which identifies the presence of a CRISPR Cas locus, identifying loci located within 10 kb of the seed comprising Open Reading Frames (ORFs) in the selected sequences, selecting therefrom loci comprising ORFs of which only a single ORF encodes a novel CRISPR effector having greater than 700 amino acids and no more than 90% homology to a known CRISPR effector. In particular embodiments, the seed is a protein that is common to the CRISPR-Cas system, such as Cas1. In further embodiments, the CRISPR array is used as a seed to identify new effector proteins.

Also, “Dimeric CRISPR RNA-guided FokI nucleases for highly specific genome editing”, Shengdar Q. Tsai, Nicolas Wyvekens, Cyd Khayter, Jennifer A. Foden, Vishal Thapar, Deepak Reyon, Mathew J. Goodwin, Martin J. Aryee, J. Keith Joung Nature Biotechnology 32(6): 569-77 (2014), relates to dimeric RNA-guided FokI Nucleases that recognize extended sequences and can edit endogenous genes with high efficiencies in human cells.

With respect to general information on CRISPR/Cas Systems, components thereof, and delivery of such components, including methods, materials, delivery vehicles, vectors, particles, and making and using thereof, including as to amounts and formulations, as well as CRISPR-Cas-expressing eukaryotic cells, CRISPR-Cas expressing eukaryotes, such as a mouse, reference is made to: U.S. Pat. Nos. 8,999,641, 8,993,233, 8,697,359, 8,771,945, 8,795,965, 8,865,406, 8,871,445, 8,889,356, 8,889,418, 8,895,308, 8,906,616, 8,932,814, and 8,945,839; US Patent Publications US 2014-0310830 (U.S. application Ser. No. 14/105,031), US 2014-0287938 A1 (U.S. application Ser. No. 14/213,991), US 2014-0273234 A1 (U.S. application Ser. No. 14/293,674), US2014-0273232 A1 (U.S. application Ser. No. 14/290,575), US 2014-0273231 (U.S. application Ser. No. 14/259,420), US 2014-0256046 A1 (U.S. application Ser. No. 14/226,274), US 2014-0248702 A1 (U.S. application Ser. No. 14/258,458), US 2014-0242700 A1 (U.S. application Ser. No. 14/222,930), US 2014-0242699 A1 (U.S. application Ser. No. 14/183,512), US 2014-0242664 A1 (U.S. application Ser. No. 14/104,990), US 2014-0234972 A1 (U.S. application Ser. No. 14/183,471), US 2014-0227787 A1 (U.S. application Ser. No. 14/256,912), US 2014-0189896 A1 (U.S. application Ser. No. 14/105,035), US 2014-0186958 (U.S. application Ser. No. 14/105,017), US 2014-0186919 A1 (U.S. application Ser. No. 14/104,977), US 2014-0186843 A1 (U.S. application Ser. No. 14/104,900), US 2014-0179770 A1 (U.S. application Ser. No. 14/104,837) and US 2014-0179006 A1 (U.S. application Ser. No. 14/183,486), US 2014-0170753 (U.S. application Ser. No. 14/183,429); US 2015-0184139 (U.S. application Ser. No. 14/324,960); Ser. No. 14/054,414 European Patent Applications EP 2 771 468 (EP13818570.7), EP 2 764 103 (EP13824232.6), and EP 2 784 162 (EP14170383.5); and PCT Patent Publications WO2014/093661 (PCT/US2013/074743), WO2014/093694 (PCT/US2013/074790), WO2014/093595 (PCT/US2013/074611), WO2014/093718 (PCT/US2013/074825), WO2014/093709 (PCT/US2013/074812), WO2014/093622 (PCT/US2013/074667), WO2014/093635 (PCT/US2013/074691), WO2014/093655 (PCT/US2013/074736), WO2014/093712 (PCT/US2013/074819), WO2014/093701 (PCT/US2013/074800), WO2014/018423 (PCT/US2013/051418), WO2014/204723 (PCT/US2014/041790), WO2014/204724 (PCT/US2014/041800), WO2014/204725 (PCT/US2014/041803), WO2014/204726 (PCT/US2014/041804), WO2014/204727 (PCT/US2014/041806), WO2014/204728 (PCT/US2014/041808), WO2014/204729 (PCT/US2014/041809), WO2015/089351 (PCT/US2014/069897), WO2015/089354 (PCT/US2014/069902), WO2015/089364 (PCT/US2014/069925), WO2015/089427 (PCT/US2014/070068), WO2015/089462 (PCT/US2014/070127), WO2015/089419 (PCT/US2014/070057), WO2015/089465 (PCT/US2014/070135), WO2015/089486 (PCT/US2014/070175), WO2015/058052 (PCT/US2014/061077), WO2015/070083 (PCT/US2014/064663), WO2015/089354 (PCT/US2014/069902), WO2015/089351 (PCT/US2014/069897), WO2015/089364 (PCT/US2014/069925), WO2015/089427 (PCT/US2014/070068), WO2015/089473 (PCT/US2014/070152), WO2015/089486 (PCT/US2014/070175), WO2016/049258 (PCT/US2015/051830), WO2016/094867 (PCT/US2015/065385), WO2016/094872 (PCT/US2015/065393), WO2016/094874 (PCT/US2015/065396), WO2016/106244 (PCT/US2015/067177).

Mention is also made of U.S. application 62/180,709, 17 Jun. 2015, PROTECTED GUIDE RNAS (PGRNAS); U.S. application 62/091,455, filed, 12 Dec. 2014, PROTECTED GUIDE RNAS (PGRNAS); U.S. application 62/096,708, 24 Dec. 2014, PROTECTED GUIDE RNAS (PGRNAS); U.S. applications 62/091,462, 12 Dec. 2014, 62/096,324, 23 Dec. 2014, 62/180,681, 17 Jun. 2015, and 62/237,496, 5 Oct. 2015, DEAD GUIDES FOR CRISPR TRANSCRIPTION FACTORS; U.S. application 62/091,456, 12 Dec. 2014 and 62/180,692, 17 Jun. 2015, ESCORTED AND FUNCTIONALIZED GUIDES FOR CRISPR-CAS SYSTEMS; U.S. application 62/091,461, 12 Dec. 2014, DELIVERY, USE AND THERAPEUTIC APPLICATIONS OF THE CRISPR-CAS SYSTEMS AND COMPOSITIONS FOR GENOME EDITING AS TO HEMATOPOETIC STEM CELLS (HSCs); U.S. application 62/094,903, 19 Dec. 2014, UNBIASED IDENTIFICATION OF DOUBLE-STRAND BREAKS AND GENOMIC REARRANGEMENT BY GENOME-WISE INSERT CAPTURE SEQUENCING; U.S. application 62/096,761, 24 Dec. 2014, ENGINEERING OF SYSTEMS, METHODS AND OPTIMIZED ENZYME AND GUIDE SCAFFOLDS FOR SEQUENCE MANIPULATION; U.S. application 62/098,059, 30 Dec. 2014, 62/181,641, 18 Jun. 2015, and 62/181,667, 18 Jun. 2015, RNA-TARGETING SYSTEM; U.S. application 62/096,656, 24 Dec. 2014 and 62/181,151, 17 Jun. 2015, CRISPR HAVING OR ASSOCIATED WITH DESTABILIZATION DOMAINS; U.S. application 62/096,697, 24 Dec. 2014, CRISPR HAVING OR ASSOCIATED WITH AAV; U.S. application 62/098,158, 30 Dec. 2014, ENGINEERED CRISPR COMPLEX INSERTIONAL TARGETING SYSTEMS; U.S. application 62/151,052, 22 Apr. 2015, CELLULAR TARGETING FOR EXTRACELLULAR EXOSOMAL REPORTING; U.S. application 62/054,490, 24 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING PARTICLE DELIVERY COMPONENTS; U.S. application 61/939,154, 12-F EB-14, SYSTEMS, METHODS AND COMPOSITIONS FOR SEQUENCE MANIPULATION WITH OPTIMIZED FUNCTIONAL CRISPR-CAS SYSTEMS; U.S. application 62/055,484, 25 Sep. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/087,537, 4 Dec. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/054,651, 24 Sep. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. application 62/067,886, 23 Oct. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. applications 62/054,675, 24 Sep. 2014 and 62/181,002, 17 Jun. 2015, DELIVERY, USE AND THERAPEUTIC APPLICATIONS OF THE CRISPR-CAS SYSTEMS AND COMPOSITIONS IN NEURONAL CELLS/TISSUES; U.S. application 62/054,528, 24 Sep. 2014, DELIVERY, USE AND THERAPEUTIC APPLICATIONS OF THE CRISPR-CAS SYSTEMS AND COMPOSITIONS IN IMMUNE DISEASES OR DISORDERS; U.S. application 62/055,454, 25 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING CELL PENETRATION PEPTIDES (CPP); U.S. application 62/055,460, 25 Sep. 2014, MULTIFUNCTIONAL-CRISPR COMPLEXES AND/OR OPTIMIZED ENZYME LINKED FUNCTIONAL-CRISPR COMPLEXES; U.S. application 62/087,475, 4 Dec. 2014 and 62/181,690, 18 Jun. 2015, FUNCTIONAL SCREENING WITH OPTIMIZED FUNCTIONAL CRISPR-CAS SYSTEMS; U.S. application 62/055,487, 25 Sep. 2014, FUNCTIONAL SCREENING WITH OPTIMIZED FUNCTIONAL CRISPR-CAS SYSTEMS; U.S. application 62/087,546, 4 Dec. 2014 and 62/181,687, 18 Jun. 2015, MULTIFUNCTIONAL CRISPR COMPLEXES AND/OR OPTIMIZED ENZYME LINKED FUNCTIONAL-CRISPR COMPLEXES; and U.S. application 62/098,285, 30 Dec. 2014, CRISPR MEDIATED IN VIVO MODELING AND GENETIC SCREENING OF TUMOR GROWTH AND METASTASIS.

Mention is made of U.S. applications 62/181,659, 18 Jun. 2015 and 62/207,318, 19 Aug. 2015, ENGINEERING AND OPTIMIZATION OF SYSTEMS, METHODS, ENZYME AND GUIDE SCAFFOLDS OF CAS9 ORTHOLOGS AND VARIANTS FOR SEQUENCE MANIPULATION. Mention is made of U.S. applications 62/181,663, 18 Jun. 2015 and 62/245,264, 22 Oct. 2015, NOVEL CRISPR ENZYMES AND SYSTEMS, U.S. applications 62/181,675, 18 Jun. 2015, 62/285,349, 22 Oct. 2015, 62/296,522, 17 Feb. 2016, and 62/320,231, 8 Apr. 2016, NOVEL CRISPR ENZYMES AND SYSTEMS, U.S. application 62/232,067, 24 Sep. 2015, U.S. application Ser. No. 14/975,085, 18 Dec. 2015, European application No. 16150428.7, U.S. application 62/205,733, 16 Aug. 2015, U.S. application 62/201,542, 5 Aug. 2015, U.S. application 62/193,507, 16 Jul. 2015, and U.S. application 62/181,739, 18 Jun. 2015, each entitled NOVEL CRISPR ENZYMES AND SYSTEMS and of U.S. application 62/245,270, 22 Oct. 2015, NOVEL CRISPR ENZYMES AND SYSTEMS. Mention is also made of U.S. application 61/939,256, 12 Feb. 2014, and WO 2015/089473 (PCT/US2014/070152), 12 Dec. 2014, each entitled ENGINEERING OF SYSTEMS, METHODS AND OPTIMIZED GUIDE COMPOSITIONS WITH NEW ARCHITECTURES FOR SEQUENCE MANIPULATION. Mention is also made of PCT/US2015/045504, 15 Aug. 2015, U.S. application 62/180,699, 17 Jun. 2015, and U.S. application 62/038,358, 17 Aug. 2014, each entitled GENOME EDITING USING CAS9 NICKASES.

### Tale Systems

As disclosed herein editing can be made by way of the transcription activator-like effector nucleases (TALENs) system. Transcription activator-like effectors (TALEs) can be engineered to bind practically any desired DNA sequence. Exemplary methods of genome editing using the TALEN system can be found for example in Cermak T. Doyle E L. Christian M. Wang L. Zhang Y. Schmidt C, et al. Efficient design and assembly of custom TALEN and other TAL effector-based constructs for DNA targeting. Nucleic Acids Res. 2011; 39:e82; Zhang F. Cong L. Lodato S. Kosuri S. Church G M. Arlotta P Efficient construction of sequence-specific TAL effectors for modulating mammalian transcription. Nat Biotechnol. 2011; 29:149-153 and U.S. Pat. Nos. 8,450,471, 8,440,431 and 8,440,432, all of which are specifically incorporated by reference.

In advantageous embodiments of the invention, the methods provided herein use isolated, non-naturally occurring, recombinant or engineered DNA binding proteins that comprise TALE monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, or “TALE monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X1-11-(X12X13)-X14-33 or 34 or 35, where the subscript indicates the amino acid position and X represents any amino acid. X12X13 indicate the RVDs. In some polypeptide monomers, the variable amino acid at position 13 is missing or absent and in such polypeptide monomers, the RVD consists of a single amino acid. In such cases the RVD may be alternatively represented as X*, where X represents X12 and (*) indicates that X13 is absent. The DNA binding domain comprises several repeats of TALE monomers and this may be represented as (X1-11-(X12X13)-X14-33 or 34 or 35)z, where in an advantageous embodiment, z is at least 5 to 40. In a further advantageous embodiment, z is at least 10 to 26.

The TALE monomers have a nucleotide binding affinity that is determined by the identity of the amino acids in its RVD. For example, polypeptide monomers with an RVD of NI preferentially bind to adenine (A), polypeptide monomers with an RVD of NG preferentially bind to thymine (T), polypeptide monomers with an RVD of HD preferentially bind to cytosine (C) and polypeptide monomers with an RVD of NN preferentially bind to both adenine (A) and guanine (G). In yet another embodiment of the invention, polypeptide monomers with an RVD of IG preferentially bind to T. Thus, the number and order of the polypeptide monomer repeats in the nucleic acid binding domain of a TALE determines its nucleic acid target specificity. In still further embodiments of the invention, polypeptide monomers with an RVD of NS recognize all four base pairs and may bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011), each of which is incorporated by reference in its entirety.

The TALE polypeptides used in methods of the invention are isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In a preferred embodiment of the invention, polypeptide monomers having RVDs RN, NN, NK, SN, NH, KN, HN, NQ, HH, RG, KH, RH and SS preferentially bind to guanine. In a much more advantageous embodiment of the invention, polypeptide monomers having RVDs RN, NK, NQ, HH, KH, RH, SS and SN preferentially bind to guanine and thereby allow the generation of TALE polypeptides with high binding specificity for guanine containing target nucleic acid sequences. In an even more advantageous embodiment of the invention, polypeptide monomers having RVDs HH, KH, NH, NK, NQ, RH, RN and SS preferentially bind to guanine and thereby allow the generation of TALE polypeptides with high binding specificity for guanine containing target nucleic acid sequences. In a further advantageous embodiment, the RVDs that have high binding specificity for guanine are RN, NH RH and KH. Furthermore, polypeptide monomers having an RVD of NV preferentially bind to adenine and guanine. In more preferred embodiments of the invention, polypeptide monomers having RVDs of H*, HA, KA, N*, NA, NC, NS, RA, and S* bind to adenine, guanine, cytosine and thymine with comparable affinity.

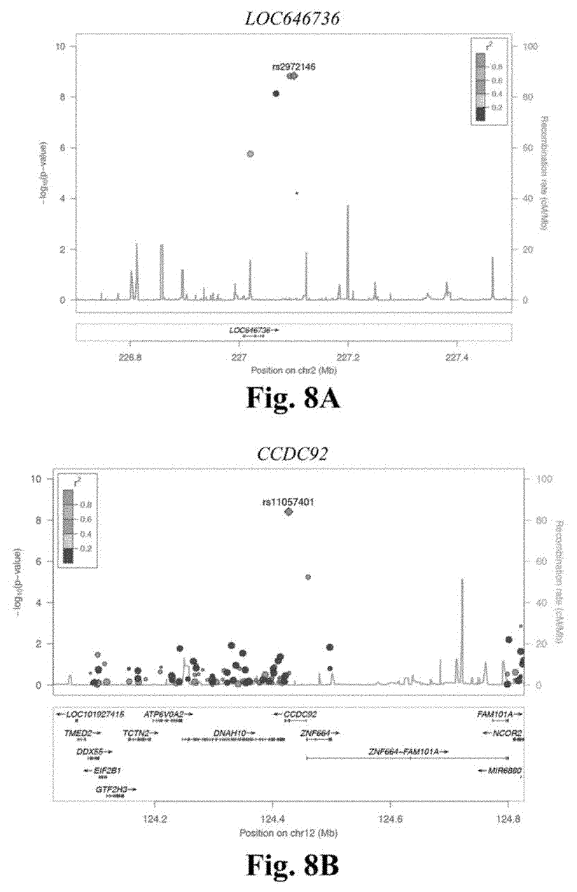

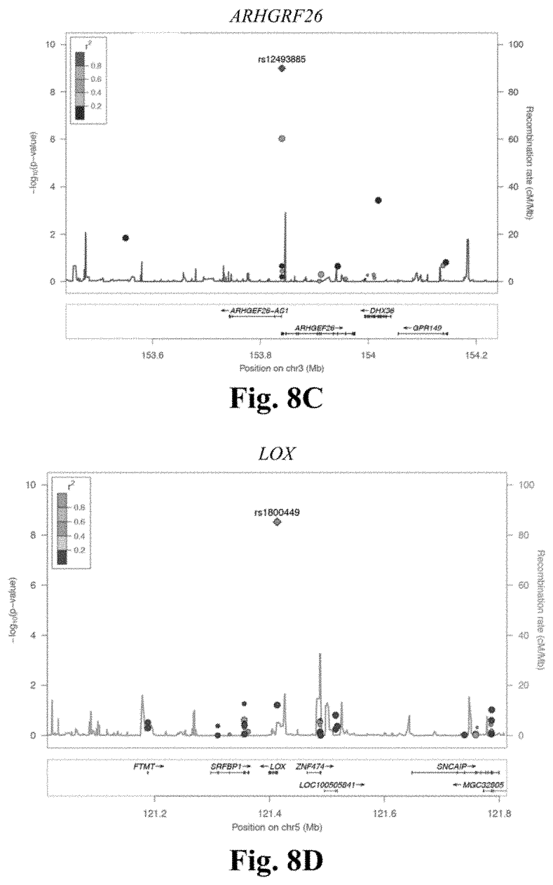

The predetermined N-terminal to C-terminal order of the one or more polypeptide monomers of the nucleic acid or DNA binding domain determines the corresponding predetermined target nucleic acid sequence to which the TALE polypeptides will bind. As used herein the polypeptide monomers and at least one or more half polypeptide monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases this region may be referred to as repeat 0. In animal genomes, TALE binding sites do not necessarily have to begin with a thymine (T) and TALE polypeptides may target DNA sequences that begin with T, A, G or C. The tandem repeat of TALE monomers always ends with a half-length repeat or a stretch of sequence that may share identity with only the first 20 amino acids of a repetitive full length TALE monomer and this half repeat may be referred to as a half-monomer (FIG. 8), which is included in the term “TALE monomer”. Therefore, it follows that the length of the nucleic acid or DNA being targeted is equal to the number of full polypeptide monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

An exemplary amino acid sequence of a N-terminal capping region is:

(SEQ ID NO: 1)
MDPIRSRTPSPARELLSGPQPDGVQPTADRGVSPPAGGPLDGLPARRTMSR
TRLPSPPAPSPAFSADSFSDLLRQFDPSLFNTSLFDSLPPFGAHHTEAATG
EWDEVQSGLRAADAPPPTMRVAVTAARPPRAKPAPRRRAAQPSDASPAAQV
DLRTLGYSQQQQEKIKPKVRSTVAQHHEALVGHGFTHAHTVALSQHPAALG
TVAVKYQDMIAALPEATHEAIVGVGKQWSGARALEALLTVAGELRGPPLQL
DTGQLLKIAKRGGVTAVEAVHAWRNALTGAPLN

An exemplary amino acid sequence of a C-terminal capping region is:

(SEQ ID NO: 2)
RPALESIVAQLSRPDPALAALTNDHLVALACLGGRPALDAVKKGLPHAPAL
IKRTNRRIPERTSHRVADHAQVVRVLGFFQCHSHPAQAFDDAMTQFGMSRH
GLLQLFRRVGVTELEARSGTLPPASQRWDRILQASGMKRAKPSPTSTQTPD
QASLHAFADSLERDLDAPSPMHEGDQTRAS

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full length capping region, while fragments that include the C-terminal 20 amino acids retain greater than 50% of the efficacy of the full length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies may be generated by any of a number of computer programs known in the art, which include but are not limited to BLAST or FASTA. Suitable computer program for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In advantageous embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4X domain or a Kruppel-associated box (KRAB) or fragments of the KRAB domain. In some embodiments the effector domain is an enhancer of transcription (i.e. an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination the activities described herein.

### 3. ZN-Finger Nucleases

Other preferred tools for genome editing for use in the context of this invention include zinc finger systems and TALE systems. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

ZFPs can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

### 4. Meganucleases

As disclosed herein editing can be made by way of meganucleases, which are endodeoxyribonucleases characterized by a large recognition site (double-stranded DNA sequences of 12 to 40 base pairs). Exemplary method for using meganucleases can be found in U.S. Pat. Nos. 8,163,514; 8,133,697; 8,021,867; 8,119,361; 8,119,381; 8,124,369; and 8,129,134, which are specifically incorporated by reference.

The present invention will be further illustrated in the following Examples which are given for illustration purposes only and are not intended to limit the invention in any way.

### EXAMPLES

### Example 1

Coronary artery disease (CAD) is a leading cause of disability and mortality worldwide (GBD 2015 Mortality and Causes of Death Collaborators, Global, regional, and national life expectancy, all-cause mortality, and cause-specific mortality for 249 causes of death, 1980-2015: a systematic analysis for the Global Burden of Disease Study 2015. Lancet 388, 1459-1544 (2016)). Genome-wide association studies (GWAS) have provided new clues to the pathophysiology for this common, complex disease. Largely using a case-control design with cases ascertained based on CAD status, published studies have highlighted at least 80 loci reaching genome-wide significance (Schunkert, H. et al., Nat Genet 43, 333-8 (2011); Deloukas, P. et al., Nat Genet 45, 25-33 (2013); CARDIoGRAMplusC4D Consortium. A comprehensive 1000 Genomes-based genome-wide association meta-analysis of coronary artery disease. Nat Genet 47, 1121-30 (2015); Myocardial Infarction Genetics and CARDIoGRAM Exome Consortia Investigators. Coding Variation in ANGPTL4, LPL, and SVEP1 and the Risk of Coronary Disease. N Engl J Med 374, 1134-44 (2016); Nioi, P. et al., N Engl J Med 374, 2131-41 (2016); Webb, T. R. et al., J Am Coll Cardiol 69, 823-836 (2017); Howson, J. M. M. et al., Nature Genetics (2017)).

Population-based biobanks such as UK Biobank offer new potential for genetic analysis of common complex diseases. New opportunities include scale, a diverse range of traits, and the ability to explore a fuller spectrum of phenotypic consequences for identified DNA variants. Leveraging the UK Biobank resource, Applicants sought to: 1) perform a genetic discovery analysis; 2) explore the phenotypic consequences and tissue-specific effects associated with CAD risk alleles; and 3) characterize the functional consequences of a risk mutation in a promising pathway.

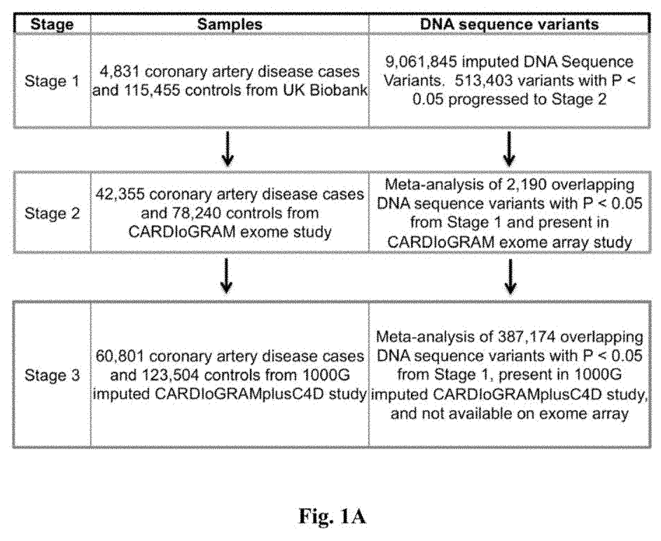

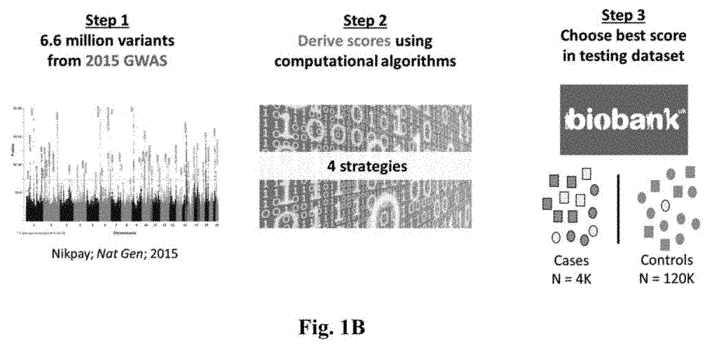

Applicants designed a three-stage GWAS (FIG. 1). In Stage 1, Applicants tested the association of DNA sequence variants with CAD in UK Biobank. In Stage 2, Applicants took forward 2,190 variants that reached nominal significance in Stage 1 (P<0.05) for meta-analysis with results from an exome-focused-array analysis in 42,355 cases and 78,240 controls (Myocardial Infarction Genetics and CARDIoGRAM Exome Consortia Investigators, Coding Variation in ANGPTL4, LPL, and SVEP1 and the Risk of Coronary Disease, N Engl J Med 374, 1134-44 (2016)). In Stage 3, Applicants took forward 387,174 variants that reached nominal significance in Stage 1 and not tested in Stage 2 for meta-analysis with results from a genome-wide imputation study in 60,801 cases and 123,504 controls (CARDIoGRAMplusC4D Consortium, A comprehensive 1000 Genomes-based genome-wide association meta-analysis of coronary artery disease, Nat Genet 47, 1121-30 (2015)). For each variant, Applicants combined statistical evidence across Stages 1 and 2 (or Stages 1 and 3) and set a statistical threshold of P<5×10-8 for genome-wide significance.

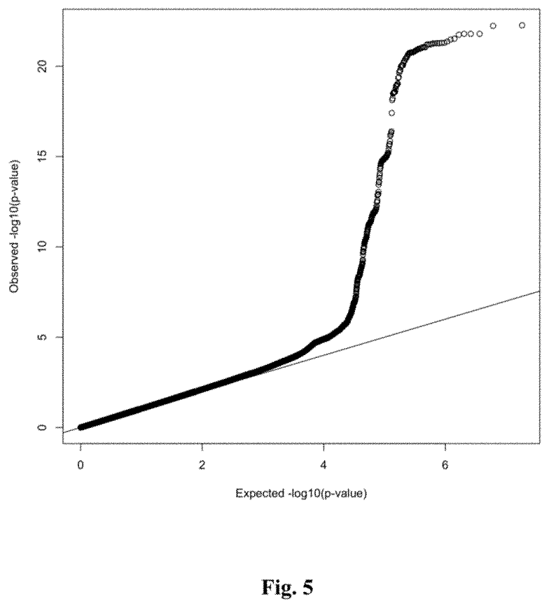

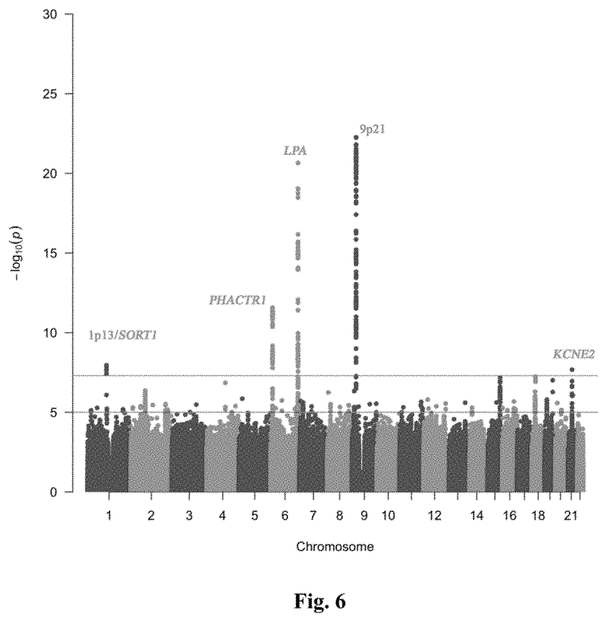

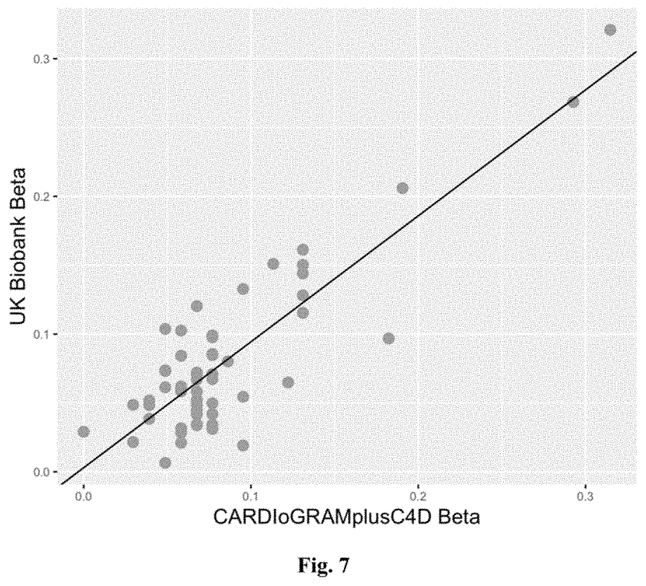

Characteristics of UK Biobank participants stratified by presence of CAD are presented in Table 1. CAD cases were more likely to be older, male, on lipid-lowering therapy, have a history of smoking, and affected with type 2 diabetes. After quality control, 9,061,845 DNA sequence variants were tested for association in 4,831 CAD patients and 115,455 controls in UK Biobank (Stage 1). A total of 269 variants at five distinct loci met the genome-wide significance threshold (P<5×10-8) (FIGS. 5 and 6). All five have been previously reported (CARDIoGRAMplusC4D Consortium. A comprehensive 1000 Genomes-based genome-wide association meta-analysis of coronary artery disease. Nat Genet 47, 1121-30 (2015); Musunuru, K. et al., Nature 466, 714-9 (2010); Myocardial Infarction Genetics Consortium et al., Nat Genet 41, 334-41 (2009); Tregouet, D. A. et al., Nat Genet 41, 283-5 (2009); Samani, N. J. et al., N Engl J Med 357, 443-53 (2007)). In UK Biobank, the 9p21/CDKN2B-AS1 variant rs4977575 (NC_000009.12:g.22124745C>G) was the top association result (49% frequency for G allele; OR=1.24; 95% CI: 1.19-1.29; P=5.40×10-23); the other four loci were 1p13/SORT1, PHACTR1, LPA, and KCNE2 (Table 2). For a set of previously reported CAD loci (CARDIoGRAMplusC4D Consortium. A comprehensive 1000 Genomes-based genome-wide association meta-analysis of coronary artery disease. Nat Genet 47, 1121-30 (2015)), Applicants compared the effect estimates from the published literature with that from the current analysis in UK Biobank and found strong positive correlation in effect sizes (13=0.92, 95% CI: 0.77-1.06; P=1.8×10-17, FIG. 7); these results validate our CAD phenotype definition in UK Biobank. A total of 513,403 variants exceeded nominal significance (P<0.05) and were taken forward to Stages 2 or 3.

TABLE 1
Characteristics of coronary artery disease
cases and controls in UK Biobank

| | Cases | Controls |
|---|---|---|
| N Individuals | 4831 | 115,455 |
| Age ± SD, years | 62.1 ± 5.9 | 56.7 ± 7.9 |
| Male, n (%) | 3908 (80%) | 53,028 (45.9%) |
| Lipid Lowering Therapy, n (%) | 3998 (82.8%) | 18,482 (16.0%) |
| Ever Smoker, n (%) | 2528 (52.3%) | 52,629 (45.6%) |
| Hypertension, n (%) | 3373 (69.8%) | 22,809 (19.6%) |
| Diabetes Mellitus, n (%) | 880 (18.2%) | 5524 (4.8%) |
| Body Mass Index ± SD, kg/m² | 29.3 ± 4.8 | 27.5 ± 4.8 |

TABLE 2
UK Biobank Stage 1 Analysis - Genome Wide Significant Loci

| SNP | Chr | Gene | Description | EA | EAF | OR | 95% CI | P |
|---|---|---|---|---|---|---|---|---|
| rs646776 | 1 | (1P13/SORT1) | downstream | T | 0.78 | 1.17 | 1.11-1.23 | 1.3 × 10⁻⁸ |
| rs9349379 | 6 | PHACTR1 | intronic | G | 0.41 | 1.15 | 1.11-1.20 | 3.4 × 10⁻¹¹ |
| rs140570886 | 6 | LPA | intronic | C | 0.02 | 1.92 | 1.68-2.20 | 2.2 × 10⁻²¹ |
| rs4977575 | 9 | (9p21/ | intergenic | G | 0.49 | 1.24 | 1.19-1.29 | 5.4 × 10⁻²³ |
| | | CDKN2B-AS1) | | | | | | |
| rs28451064 | 21 | (KCNE2) | intergenic | A | 0.13 | 1.18 | 1.11-1.25 | 2.1 × 10⁻⁸ |
| | | Gene Desert | | | | | | |

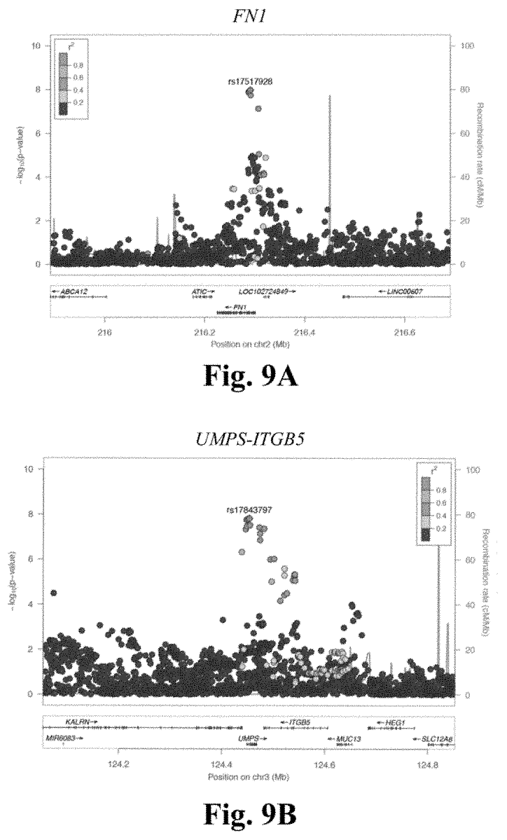

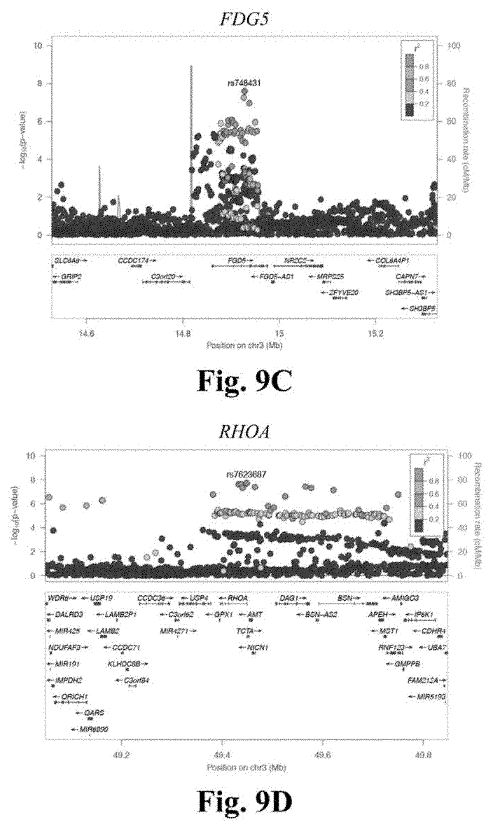

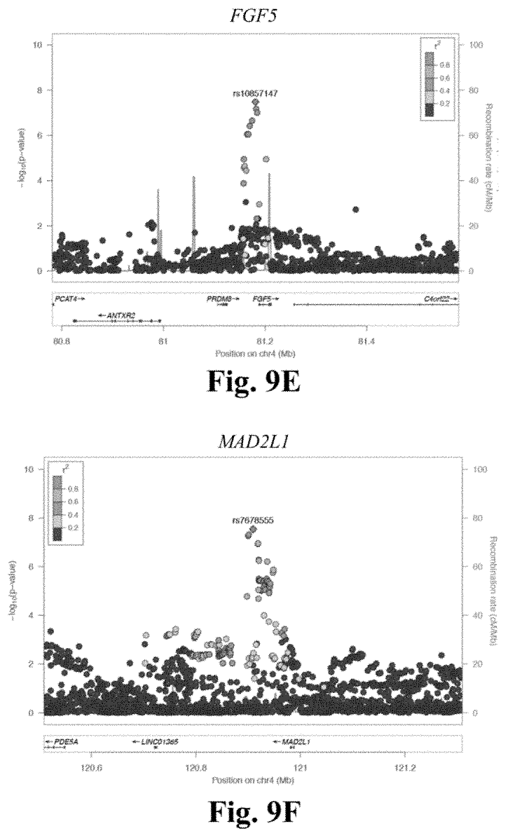

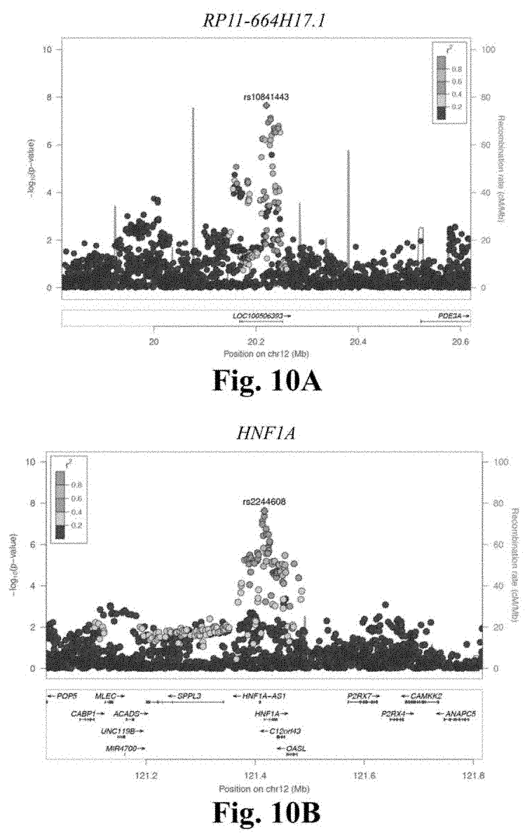

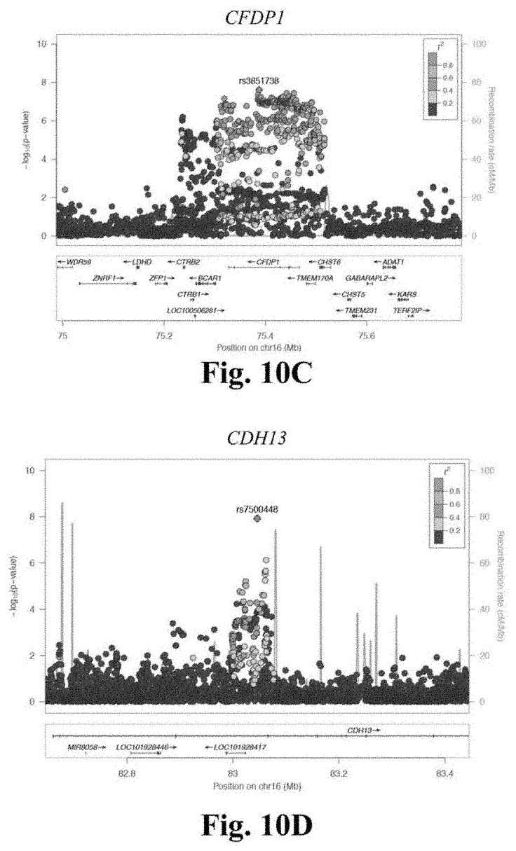

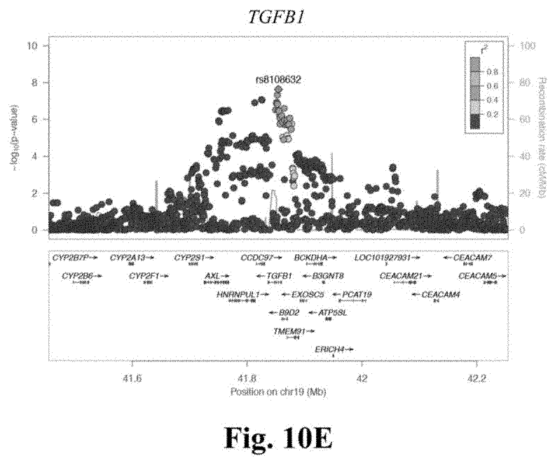

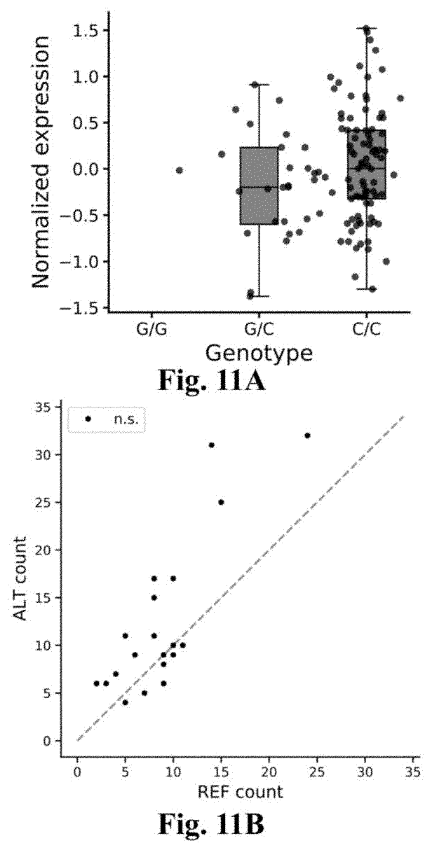

After meta-analysis, 15 new loci exceeded genome-wide significance (Tables 3-4), bringing the total number of established CAD loci to 95. One of the 15 loci (HNF1A) has since been reported in Howson, J. M. M. et al., Nature Genetics (2017). Effect allele frequencies of the 15 newly identified loci ranged from 13% to 86%, with effect sizes ranging from 1.05 to 1.08. Descriptions of relevant loci appear in Table 5, and regional association plots for novel CAD loci are shown in FIGS. 8-10.

TABLE 3
Table 3 - New loci from analysis of UK Biobank and CARDIoGRAM exome study.

| | | | | | | | | Stage 2 | | | | |
|---|---|---|---|---|---|---|---|---|---|---|---|---|
| UK Biobank | | | | | | | | Exome Study | | Combined | | |
| Lead Variant | Chr | Gene | Description | EA | EAF | OR | P | OR | P | OR | 95% CI | P |
| rs2972146 | 2 | (LOC646736) | intergenic | T | 0.65 | 1.07 | 0.0011 | 1.05 | 2.01 × 10⁻⁷ | 1.06 | 1.04-1.07 | 1.46 × 10⁻⁹ |
| rs12493885 | 3 | ARHGEF26 | missense | C | 0.85 | 1.07 | 0.039 | 1.09 | 8.28 × 10⁻⁹ | 1.08 | 1.06-1.11 | 1.02 × 10⁻⁹ |
| (p.Val29Leu) | | | | | | | | | | | | |
| rs1800449 | 5 | LOX | missense | T | 0.17 | 1.09 | 0.0039 | 1.07 | 1.72 × 10⁻⁷ | 1.07 | 1.05-1.09 | 2.99 × 10⁻⁹ |
| rs11057401 | 12 | CCDC92 | missense | T | 0.69 | 1.08 | 0.001 | 1.05 | 4.32 × 10⁻⁷ | 1.06 | 1.04-1.08 | 3.88 × 10⁻⁹ |
| (p.Ser70Cys) | | | | | | | | | | | | |

*Genes for variants that are outside the transcript boundary of the protein-coding gene are shown in parentheses [eg, (LOC646736)].
Chr = Chromosome, CI = Confidence Interval, EA = Effect Allele, EAF = Effect Allele Frequency, OR = Odds Ratio.

TABLE 4
Table 4 - New Loci from analysis of UK Biobank and CARDIoGRAMplusC4D 1000G imputation study.

| | | | | | | | | Stage 3 | | | | |
|---|---|---|---|---|---|---|---|---|---|---|---|---|
| | | | | | | | | 1000G | | | | |
| UK Biobank | | | | | | | | Imputed Study | | Combined | | |
| Lead Variant | Chr | Gene | Description | EA | EAF | OR | P | OR | P | OR | 95% CI | P |
| rs17517928 | 2 | FN1 | intronic | C | 0.75 | 1.08 | 0.0026 | 1.06 | 5.14 × 10⁻⁷ | 1.06 | 1.04-1.08 | 1.06 × 10⁻⁸ |
| rs17843797 | 3 | UMPS- | intronic | G | 0.13 | 1.11 | 0.00019 | 1.07 | 2.43 × 10⁻⁶ | 1.07 | 1.05-1.10 | 1.52 × 10⁻⁸ |
| | | ITGB5 | | | | | | | | | | |
| rs748431 | 3 | FGD5 | intronic | G | 0.36 | 1.04 | 0.042 | 1.05 | 2.14 × 10⁻⁷ | 1.05 | 1.03-1.07 | 2.63 × 10⁻⁸ |
| rs7623687 | 3 | RHOA | intronic | A | 0.86 | 1.09 | 0.0073 | 1.07 | 5.22 × 10⁻⁷ | 1.08 | 1.05-1.10 | 2.00 × 10⁻⁸ |
| rs10857147 | 4 | (FGF5) | regulatory | T | 0.29 | 1.06 | 0.014 | 1.06 | 5.83 × 10⁻⁷ | 1.06 | 1.04-1.08 | 3.39 × 10⁻⁸ |
| | | | region | | | | | | | | | |
| rs7678555 | 4 | (MAD2L1) | intergenic | C | 0.29 | 1.06 | 0.027 | 1.06 | 3.26 × 10⁻⁷ | 1.06 | 1.04-1.08 | 2.91 × 10⁻⁸ |
| rs10841443 | 12 | RP11-664H17.1 | intronic | G | 0.67 | 1.06 | 0.0073 | 1.05 | 5.81 × 10⁻⁷ | 1.05 | 1.03-1.07 | 2.23 × 10⁻⁸ |
| rs2244608 | 12 | HNF1A | intronic | G | 0.32 | 1.07 | 0.003 | 1.05 | 1.02 × 10⁻⁶ | 1.05 | 1.03-1.07 | 2.41 × 10⁻⁸ |
| rs3851738 | 16 | CFDP1 | intronic | C | 0.6 | 1.07 | 0.00089 | 1.05 | 1.88 × 10⁻⁶ | 1.05 | 1.03-1.07 | 2.43 × 10⁻⁸ |
| rs7500448 | 16 | CDH13 | intronic | A | 0.75 | 1.1 | 0.00016 | 1.06 | 2.11 × 10⁻⁶ | 1.06 | 1.04-1.09 | 1.20 × 10⁻⁸ |
| rs8108632 | 19 | TGFB1 | intronic | T | 0.41 | 1.06 | 0.011 | 1.05 | 4.76 × 10⁻⁷ | 1.05 | 1.03-1.07 | 2.35 × 10⁻⁸ |

* Genes for variants that are outside the transcript boundary of the protein-coding gene are shown in parentheses [e.g., (FGF5)].
1000G = 1000 Genomes, Chr = Chromosome, CI = Confidence Interval, EA = Effect Allele, EAF = Effect Allele Frequency, OR = Odds Ratio.

TABLE 5
Descriptions of novel loci and supportive evidence suggesting causal
genes. *Genes located within 500 Kb window of lead variant. **GTEx cis-eQTLs are taken from
gtexportal.org and are limited to those with P < 5 × 10⁻⁸. ***Phenotypes were declared to be
significantly associated with the risk variant if they met a Bonferroni corrected P value of <
0.00013; PMID references denote whether the association has been previously reported at the
time of analysis. Abbreviations: BMI, Body Mass Index; CAD, Coronary Artery Disease; eGFR,
Estimated Glomerular Filtration Rate; crea, Creatinine; HDL, High Density Lipoprotein
Cholesterol; LDL, Low Density Lipoprotein Cholesterol; MI, Myocardial Infarction.

| | | Prior | | Significant | |
|---|---|---|---|---|---|
| | | Murine/Functional | GTEx cis- | PheWAS | Candidate |
| | | Evidence | eQTLs across | Associations | Causal |
| Variant | Genes at Locus* | [Reference] | all Tissues** | [Reference]*** | Gene(s) |
| rs17517928 | FN1, ATIC, | FN1-null mice | | Height [PMID: | |
| | LOC102724849, | demonstrate larger | | 25282103] | |
| | ABCA12, | infarction areas | | | |
| | LINC00607 | following transient | | | |
| | | focal cerebral | | | |
| | | ischemia [PMID: | | | |
| | | 11231631]. | | | |
| rs2972146 | LOC646736, | Islets from IRS-1 | IRS1 | Fasting Insulin | IRS1 |
| | IRS1, MIR5702 | knockout mice | | Adjusted for BMI | |
| | | exhibit marked | | [PMID: 22581228], | |
| | | insulin secretory | | Body Fat Percentage | |
| | | defects [PMID: | | [PMID: 26833246], | |
| | | 10606633]. | | Adiponectin [PMID: | |
| | | | | 22479202], Type 2 | |
| | | | | Diabetes [PMID: | |
| | | | | 22885922], HDL | |
| | | | | Cholesterol [PMID: | |
| | | | | 24097068], | |
| | | | | Triglycerides [PMID: | |
| | | | | 24097068] | |
| rs17843797 | UMPS, ITGB5, | | | Body Fat Percentage | |
| | KALRN, | | | | |
| | MIR6083, | | | | |
| | MUC13, HEG1, | | | | |
| | SLC12A8, | | | | |
| | MIR5092 | | | | |
| rs748431 | FGD5, FGD5- | | | | |
| | AS1, NR2C2, | | | | |
| | ZFYVE20, | | | | |
| | COL6A4P1, | | | | |
| | CAPN7, | | | | |
| | SH3BP5, | | | | |
| | SH3BP5-AS1 | | | | |
| rs7623687 | RHOA, | | | Inflammatory Bowel | |
| | ARIH2OS, | | | Disease [PMID: | |
| | ARIH2, P4HTM | | | 26192919] | |
| | WDR6, | | | | |
| | DALRD3, | | | | |
| | MIR425, | | | | |
| | NDUFAF3, | | | | |
| | MIR191, | | | | |
| | IMPDH2, | | | | |
| | QRICH1, QARS, | | | | |
| | MIR6890, | | | | |
| | USP19, LAMB2, | | | | |
| | LAMB2P1, | | | | |
| | CCDC71, | | | | |
| | KLHDC8B, | | | | |
| | C3or184, | | | | |
| | CCDC36, | | | | |
| | C3orf62, | | | | |
| | MIR4271, USP4, | | | | |
| | GPX1, TCTA, | | | | |
| | AMT, NICNL | | | | |
| | DAG1, | | | | |
| | BSN-AS2, BSN, | | | | |
| | APEH, MST1, | | | | |
| | RNF123, | | | | |
| | AMIGO3, | | | | |
| | GMPPB, IP6K1, | | | | |
| | CDHR4, | | | | |
| | FAM212A, | | | | |
| | UBA7, MIR5193, | | | | |
| | TRAIP, CAMKV, | | | | |
| | MST1R, MON1A | | | | |
| rs12493885 | ARHGEF26, | ARHGEF26 −/− mice | ARHGEF26- | | ARHGEF26 |
| (p.V29L) | ARHGEF26- | when crossed with | AS1, | | |
| | AS1, DHX36, | atherosclerosis- | ARHGEF26, | | |
| | GPR149 | prone APOE null | DHX36 | | |
| | | mice, display less | | | |
| | | aortic | | | |
| | | atherosclerosis | | | |
| | | [PMID: 23372835]. | | | |
| rs10857147 | FGF5, PRDM8, | | | Systolic Blood Pressure | |
| | PCAT4 ANTXR2, | | | [PMID: 21909115], | |
| | C4orf22 | | | Diastolic Blood | |
| | | | | Pressure [PMID: | |
| | | | | 21909115], eGFRcrea | |
| | | | | [PMID: 26831199] | |
| rs7678555 | MAD2L1, | Family-based | | | |
| | LOC645513, | exome sequencing | | | |
| | PDE5A | and luciferase-based | | | |
| | LINC01365 | in vitro analysis | | | |
| | | suggests that | | | |
| | | missense mutations | | | |
| | | in PDE5A may | | | |
| | | confer CAD risk | | | |
| | | through a gain of | | | |
| | | PDE5A function | | | |
| | | [PMID: 24213632, | | | |
| | | PMCID: | | | |
| | | PMC4565074]. | | | |
| rs1800449 | LOX FTMT, | Induction of MI in | | | LOX |
| | SRFBP1, | C57BL/6 mice by | | | |
| | ANF474, | ligation of the left | | | |
| | LOC100505841, | anterior descending | | | |
| | SNCAIP, | coronary artery | | | |
| | MGC32805 | resulted in strongly | | | |
| | | increased LOX | | | |
| | | expression and | | | |
| | | resulted in a | | | |
| | | significant | | | |
| | | accumulation of | | | |
| | | mature collagen | | | |
| | | fibers in the | | | |
| | | infarcted area | | | |
| | | [PMID: 16642001, | | | |
| | | 26260798]. | | | |
| rs10841443 | RP11-664H17.1, | Missense mutations | | Diastolic Blood | PDE3A |
| | PDE3A | in PDE3A have been | | Pressure [PMID: | |
| | | demonstrated to | | 26390057] | |
| | | cause an autosomal | | | |
| | | dominant form of | | | |
| | | hypertension and | | | |
| | | induction of thse | | | |
| | | mutations resulted | | | |
| | | in alterations in | | | |
| | | vascular remodeling | | | |
| | | phenotypes in | | | |
| | | vascular smooth | | | |
| | | muscle cells in vitro | | | |
| | | [PMID: 25961942]. | | | |
| rs2244608 | HNF1A, | | | LDL Cholesterol | |
| | DYNLL1, | | | [PMID: 24097068], | |
| | DYNLL1-AS1, | | | Total Cholesterol | |
| | COQ5, RNF10, | | | [PMID: 24097068] | |
| | POP5, CABP1, | | | | |
| | MLEC, | | | | |
| | UNC119B, | | | | |
| | MIR4700, | | | | |
| | ACADS, SPPL3, | | | | |
| | HNF1A-AS1, | | | | |
| | C12orf43, OASL, | | | | |
| | P2RX7, P2RX4, | | | | |
| | CAMKK2, | | | | |
| | ANAPC5, | | | | |
| | RNF34, KDM2B, | | | | |
| | MIR7107 | | | | |
| rs11057401 | CCDC92, | siRNA knockdown | CCDC92, | Body Fat Percentage, | CCDC92, |
| (p.S70C) | SNRNP35, | of CCCD92 and | DNAH10OS, | Waist Hip Ratio | DNAH10 |
| | RILPL1, | DNAH10 in | RP11- | Adjusted for BMI | |
| | MIR3908, | adipocytes, genes | 380L11.4 | [PMID: 25673412], | |
| | LOC101927415, | implicated across | | Adiponectin [PMID: | |
| | TMED2, | variety of | | 22479202], HDL | |
| | DDX55, EIF2B1, | cardiometabolic | | Cholesterol [PMID: | |
| | GTF2H3, | phenotypes | | 24097068], | |
| | TCTN2, | associated with | | Triglycerides [PMID: | |
| | ATP6V0A2, | insulin resistance, | | 24097068] | |
| | DNAH10, | resulted in a | | | |
| | ZNF664, | decreased capacity | | | |
| | ZNF664- | for lipid | | | |
| | FAM101A, | accumulation | | | |
| | FAH101A, | [PMID: 27841877, | | | |
| | NCOR2, | 25673412]. | | | |
| | MIR6880 | | | | |
| rs3851738 | CFDP1, | | BCAR1, | Height [PMID: | |
| | WDR59, ZNRF1, | | CFDP1, | 25282103], Systolic | |
| | LDHD, ZFP1, | | RP11- | Blood Pressure [PMID: | |
| | CTRB2, CTRB1, | | 252K23.2 | 27841878] | |
| | LOC100506281, | | | | |
| | BCAR1, | | | | |
| | TMEM170A, | | | | |
| | CHST6, CHST5, | | | | |
| | TMEM231, | | | | |
| | GABARAPL2, | | | | |
| | ADAT1, KARS, | | | | |
| | TERF2IP | | | | |
| rs7500448 | CDH13, | CDH13 deficient | CDH13 | Adiponectin [PMID: | CDH13 |
| | MIR8058, | mice demonstrated | | 22479202] | |
| | LOC101928446, | increased infarct | | | |
| | LOC101928417 | size following left | | | |
| | | anterior descending | | | |
| | | artery ligtation, | | | |
| | | similar to that in | | | |
| | | seen adiponectin- | | | |
| | | null mice [PMID: | | | |
| | | 21041950]. | | | |
| rs8108632 | TGFB1, | | | | |
| | CYP2A7, | | | | |
| | CYP2G1P, | | | | |
| | CYP2B7P, | | | | |
| | CYP2B6, | | | | |
| | CYP2A13, | | | | |
| | CYP2F1, | | | | |
| | CYP2S1, AXL, | | | | |
| | HNRNPUL1, | | | | |
| | CCDC97, B9D2, | | | | |
| | TMEM91, | | | | |
| | EXOSC5, | | | | |
| | BCKDHA, | | | | |
| | B3GNT8, | | | | |
| | ATP5SL, | | | | |
| | ERICH4, | | | | |
| | PCAT19, | | | | |
| | LOC101927931, | | | | |
| | CEACAM21, | | | | |
| | CEACAM4, | | | | |
| | CEACAM7, | | | | |
| | CEACAM5, | | | | |
| | CEACAM6, | | | | |
| | CEACAM3, | | | | |
| | LYPD4, | | | | |
| | DMRTC2 | | | | |

To move from these 15 DNA sequence variants to biologic insights, Applicants took two approaches: phenome-wide association scanning and functional analysis. Understanding the full spectrum of phenotypic consequences of a given DNA sequence variant may shed light on the mechanism by which a variant/gene leads to disease. Termed a ‘phenome-wide association study’ or “PheWAS”, this approach tests the association of a mapped disease variant with a broad range of human phenotypes (Denny, J. C. et al., Nat Biotechnol 31, 1102-10 (2013)). In collaboration with Genomics plc, Applicants conducted a PheWAS combining UK Biobank data, mRNA transcript phenotypes in the Genotype-Tissue Expression Project (GTEx) dataset (Aguet, F. et al. Local genetic effects on gene expression across 44 human tissues. bioRxiv (2016)), and an integrated set of GWAS results from a variety of publicly available sources (Global Lipids Genetics Consortium et al., Nat Genet 45, 1274-83 (2013); Manning, A. K. et al., Nat Genet 44, 659-69 (2012); Prokopenko, I. et al., PLoS Genet 10, e1004235 (2014); Wood, A. R. et al., Nat Genet 46, 1173-86 (2014); Berndt, S. I. et al., Nat Genet 45, 501-12 (2013); Pattaro, C. et al., Nat Commun 7, 10023 (2016); Liu, J. Z. et al., Nat Genet 47, 979-86 (2015); Dastani, Z. et al., PLoS Genet 8, e1002607 (2012); Morris, A. P. et al., Nat Genet 44, 981-90 (2012)).

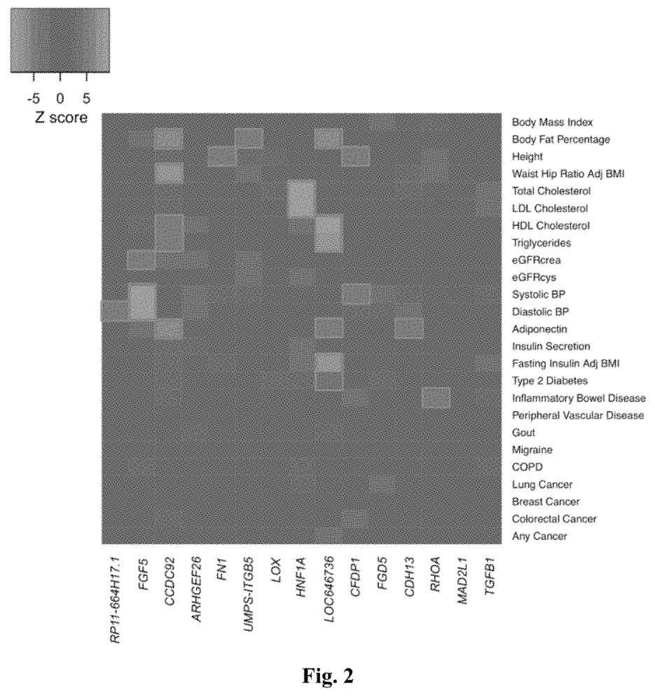

Applicants found that several of the newly identified DNA sequence variants correlated with a range of human traits (FIG. 2, Tables 6-7). For example, the intronic variant rs10841443 within RP11-664H17.1 is in close proximity to PDE3A, a phosphodiesterase previously implicated in an autosomal dominant form of hypertension (Maass, P. G. et al., Nat Genet 47, 647-53 (2015)). PheWAS showed an association for this variant with diastolic blood pressure (Kato, N. et al., Nat Genet 47, 1282-93 (2015)), suggesting that this locus may be acting through hypertension. The variant rs2244608 within HNFIA has been previously associated with LDL cholesterol, a causal path to atherosclerosis (Global Lipids Genetics Consortium et al., Nat Genet 45, 1274-83 (2013)). The variant rs7500448 within CDH13 (encoding Cadherin 13 or T-Cadherin), a vascular adiponectin receptor implicated in hypertensive and insulin resistance biology (Chung, C. M. et al., Diabetes 60, 2417-23 (2011)), associates with plasma adiponectin levels. Variant rs2972146 is downstream of IRS1 (encoding the insulin receptor substrate-1 gene (Morris, A. P. et al., Nat Genet 44, 981-90 (2012))) and is a cis-eQTL for IRS1 expression in adipose tissue. rs2972146 associates with a range of phenotypes seen in the setting of insulin resistance including HDL cholesterol, triglycerides, adiponectin, fasting insulin, and type 2 diabetes.

TABLE 6
Table 6 - Genome-wide significant variant-gene cis-eQTL pairs for 15 novel CAD risk
variants queried in GTEx Consortium Project Data, aligned to the CAD risk allele.

| | | Alleles | | cis-eQTL | P | Effect | |
|---|---|---|---|---|---|---|---|
| Variant | Chr. | Effect/Other | Gencode ID | Gene | value | Size | Tissue |
| rs2972146 | 2 | T/G | ENSG00000169047.5 | IRS1 | 2.40E−08 | −0.3 | Adipose - |
| | | | | | | | Subcutaneous |
| rs12493885 | 3 | C/G | ENSG00000243069.3 | ARHGEF26- | 1.30E−15 | 0.73 | Thyroid |
| | | | | AS1 | | | |
| rs12493885 | 3 | C/G | ENSG00000114790.8 | ARHGEF26 | 2.20E−11 | 0.45 | Artery - Tibial |
| rs12493885 | 3 | C/G | ENSG00000243069.3 | ARHGEF26- | 1.30E−09 | −0.43 | Nerve - Tibial |
| | | | | AS1 | | | |
| rs12493885 | 3 | C/G | ENSG00000174953.9 | DHX36 | 1.80E−09 | −0.29 | Heart - Left |
| | | | | | | | Ventricle |
| rs12493885 | 3 | C/G | ENSG00000114790.8 | ARHGEF26 | 1.70E−08 | 0.32 | Adipose - |
| | | | | | | | Subcutaneous |
| rs12493885 | 3 | C/G | ENSG00000174953.9 | DHX36 | 2.40E−08 | −0.39 | Esophagus - |
| | | | | | | | Gastroesophagea |
| | | | | | | | 1 Junction |
| rs11057401 | 12 | T/A | ENSG00000119242.4 | CCDC92 | 7.10E−17 | −0.53 | Heart - Left |
| | | | | | | | Ventricle |
| rs11057401 | 12 | T/A | ENSG00000250091.2 | DNAH10OS | 1.50E−14 | −0.51 | Esophagus - |
| | | | | | | | Muscularis |
| rs11057401 | 12 | T/A | ENSG00000270028.1 | RP11- | 5.90E−14 | −0.55 | Esophagus - |
| | | | | 380L11.4 | | | Muscularis |
| rs11057401 | 12 | T/A | ENSG00000250091.2 | DNAH10OS | 4.00E−12 | −0.32 | Artery - Tibial |
| rs11057401 | 12 | T/A | ENSG00000179195.11 | ZNF664 | 3.20E−11 | 0.29 | Thyroid |
| rs11057401 | 12 | T/A | ENSG00000270028.1 | RP11- | 6.10E−10 | −0.4 | Artery - Tibial |
| | | | | 380L11.4 | | | |
| rs11057401 | 12 | T/A | ENSG00000250091.2 | DNAH10OS | 8.60E−10 | −0.49 | Heart - Left |
| | | | | | | | Ventricle |
| rs11057401 | 12 | T/A | ENSG00000119242.4 | CCDC92 | 1.10E−09 | −0.34 | Adipose - |
| | | | | | | | Subcutaneous |
| rs11057401 | 12 | T/A | ENSG00000119242.4 | CCDC92 | 2.70E−08 | −0.4 | Adipose - |
| | | | | | | | Visceral |
| | | | | | | | (Omentum) |
| rs3851738 | 16 | C/G | ENSG00000261783.1 | RP11- | 7.60E−20 | −0.66 | Thyroid |
| | | | | 252K23.2 | | | |
| rs3851738 | 16 | C/G | ENSG00000261783.1 | RP11- | 1.10E−19 | −0.71 | Cells - |
| | | | | 252K23.2 | | | Transformed |
| | | | | | | | fibroblasts |
| rs3851738 | 16 | C/G | ENSG00000261783.1 | RP11- | 1.70E−19 | −0.87 | Adipose - |
| | | | | 252K23.2 | | | Visceral |
| | | | | | | | (Omentum) |
| rs3851738 | 16 | C/G | ENSG00000050820.12 | BCAR1 | 1.70E−16 | −0.48 | Esophagus - |
| | | | | | | | Mucosa |
| rs3851738 | 16 | C/G | ENSG00000261783.1 | RP11- | 2.60E−15 | −0.62 | Esophagus - |
| | | | | 252K23.2 | | | Mucosa |
| rs3851738 | 16 | C/G | ENSG00000153774.4 | CFDP1 | 5.10E−15 | −0.34 | Cells - |
| | | | | | | | Transformed |
| | | | | | | | fibroblasts |
| rs3851738 | 16 | C/G | ENSG00000261783.1 | RP11- | 1.70E−14 | −0.56 | Lung |
| | | | | 252K23.2 | | | |
| rs3851738 | 16 | C/G | ENSG00000261783.1 | RP11- | 5.00E−13 | −0.66 | Artery - Aorta |
| | | | | 252K23.2 | | | |
| rs3851738 | 16 | C/G | ENSG00000261783.1 | RP11- | 5.60E−13 | −0.54 | Artery - Tibial |
| | | | | 252K23.2 | | | |
| rs3851738 | 16 | C/G | ENSG00000261783.1 | RP11- | 7.60E−13 | −0.54 | Nerve - Tibial |
| | | | | 252K23.2 | | | |
| rs3851738 | 16 | C/G | ENSG00000261783.1 | RP11- | 1.50E−12 | −0.5 | Adipose - |
| | | | | 252K23.2 | | | Subcutaneous |
| rs3851738 | 16 | C/G | ENSG00000050820.12 | BCAR1 | 8.30E−10 | 0.2 | Artery - Tibial |
| rs3851738 | 16 | C/G | ENSG00000261783.1 | RP11- | 1.10E−09 | −0.45 | Skin - Sun |
| | | | | 252K23.2 | | | Exposed (Lower |
| | | | | | | | leg) |
| rs3851738 | 16 | C/G | ENSG00000261783.1 | RP11- | 1.30E−09 | −0.56 | Esophagus - |
| | | | | 252K23.2 | | | Muscularis |
| rs3851738 | 16 | C/G | ENSG00000050820.12 | BCAR1 | 7.70E−09 | 0.24 | Artery - Aorta |
| rs3851738 | 16 | C/G | ENSG00000261783.1 | RP11- | 1.20E−08 | −0.43 | Whole Blood |
| | | | | 252K23.2 | | | |
| rs3851738 | 16 | C/G | ENSG00000261783.1 | RP11- | 2.80E−08 | −0.65 | Adrenal Gland |
| | | | | 252K23.2 | | | |
| rs3851738 | 16 | C/G | ENSG00000261783.1 | RP11- | 4.80E−08 | −0.5 | Breast - |
| | | | | 252K23.2 | | | Mammary Tissue |
| rs7500448 | 16 | A/G | ENSG00000140945.11 | CDH13 | 9.60E−11 | 0.46 | Artery - Aorta |

Abbreviations: Chr, chromosome; eQTL, expression quantitative trait locus; GTEx, genotype-tissue expression.

TABLE 7
Table 7 - Phenome-wide association results for the 15 novel CAD variants.

| | | | | | UK | | | | | | |
|---|---|---|---|---|---|---|---|---|---|---|---|
| | | | | | Biobank | | | | | | |
| | | | | | Allele 1 | | | P | | | Beta |
| Variant | Gene | Chr | Allele1 | Allele2 | Frequency | Beta | SE | Value | Phenotype | Consortium | Units |
| rs17517928 | FN1 | 2 | C | T | 0.75 | 0.018 | 0.007 | 0.009 | Fasting Insulin Adj | MAGIC | Std Dev |
| | | | | | | | | | BMI | | |
| rs17517928 | FN1 | 2 | C | T | 0.75 | 0.007 | 0.005 | 0.169 | Body Fat Percentage | UK | Std Dev |
| | | | | | | | | | | Biobank | |
| rs17517928 | FN1 | 2 | C | T | 0.75 | 0.007 | 0.005 | 0.147 | Waist Hip Ratio Adj | UK | Std Dev |
| | | | | | | | | | BMI | Biobank | |
| rs17517928 | FN1 | 2 | C | T | 0.75 | 0.016 | 0.003 | 1.19E−06 | Height | GIANT | Std Dev |
| rs17517928 | FN1 | 2 | C | T | 0.75 | 0.000 | 0.010 | 0.974 | Adiponectin | ADIPOGen | Std Dev |
| rs17517928 | FN1 | 2 | C | T | 0.75 | −0.003 | 0.010 | 0.767 | Insulin Secretion | MAGIC | Std Dev |
| rs17517928 | FN1 | 2 | C | T | 0.75 | −0.005 | 0.006 | 0.325 | Low Density | GLGC | Std Dev |
| | | | | | | | | | Lipoprotein | | |
| | | | | | | | | | Cholesterol | | |
| rs17517928 | FN1 | 2 | C | T | 0.75 | 0.014 | 0.019 | 0.460 | Inflammatory Bowel | IIBDGC | ln(OR) |
| | | | | | | | | | Disease | | |
| rs17517928 | FN1 | 2 | C | T | 0.75 | −0.017 | 0.009 | 0.056 | eGFRcys | CKDGen | mL/min/ |
| | | | | | | | | | | | 1.73 m2 |
| rs17517928 | FN1 | 2 | C | T | 0.75 | −0.006 | 0.005 | 0.250 | Total Cholesterol | GLGC | Std Dev |
| rs17517928 | FN1 | 2 | C | T | 0.75 | 0.020 | 0.023 | 0.382 | Type 2 Diabetes | DIAGRAM | ln(OR) |
| rs17517928 | FN1 | 2 | C | T | 0.75 | 0.001 | 0.005 | 0.915 | High Density | GLGC | Std Dev |
| | | | | | | | | | Lipoprotein | | |
| | | | | | | | | | Cholesterol | | |
| rs17517928 | FN1 | 2 | C | T | 0.75 | −0.004 | 0.005 | 0.456 | Triglycerides | GLGC | Std Dev |
| rs17517928 | FN1 | 2 | C | T | 0.75 | 0.003 | 0.004 | 0.549 | eGFRcrea | CKDGen | mL/min/ |
| | | | | | | | | | | | 1.73 m2 |
| rs17517928 | FN1 | 2 | C | T | 0.75 | −0.059 | 0.032 | 0.065 | Body Mass Index | GIANT | ln(OR) |
| rs17517928 | FN1 | 2 | C | T | 0.75 | 0.303 | 0.096 | 0.002 | Systolic BP | UK | mmHg |
| | | | | | | | | | | Biobank | |
| rs17517928 | FN1 | 2 | C | T | 0.75 | 0.005 | 0.054 | 0.922 | Diastolic BP | UK | mmHg |
| | | | | | | | | | | Biobank | |
| rs17517928 | FN1 | 2 | C | T | 0.75 | 0.048 | 0.065 | 0.460 | Peripheral Vascular | UK | ln(OR) |
| | | | | | | | | | Disease | Biobank | |
| rs17517928 | FN1 | 2 | C | T | 0.75 | −0.030 | 0.042 | 0.481 | Gout | UK | ln(OR) |
| | | | | | | | | | | Biobank | |
| rs17517928 | FN1 | 2 | C | T | 0.75 | −0.025 | 0.030 | 0.417 | Migraine | UK | ln(OR) |
| | | | | | | | | | | Biobank | |
| rs17517928 | FN1 | 2 | C | T | 0.75 | 0.031 | 0.035 | 0.385 | COPD | UK | ln(OR) |
| | | | | | | | | | | Biobank | |
| rs17517928 | FN1 | 2 | C | T | 0.75 | −0.078 | 0.152 | 0.607 | Lung Cancer | UK | ln(OR) |
| | | | | | | | | | | Biobank | |
| rs17517928 | FN1 | 2 | C | T | 0.75 | −0.045 | 0.035 | 0.203 | Breast Cancer | UK | ln(OR) |
| | | | | | | | | | | Biobank | |
| rs17517928 | FN1 | 2 | C | T | 0.75 | 0.101 | 0.071 | 0.151 | Colorectal Cancer | UK | ln(OR) |
| | | | | | | | | | | Biobank | |
| rs17517928 | FN1 | 2 | C | T | 0.75 | 0.015 | 0.018 | 0.409 | Any Cancer | UK | ln(OR) |
| | | | | | | | | | | Biobank | |
| rs2972146 | LOC646736 | 2 | T | G | 0.65 | 0.045 | 0.006 | 6.39E−14 | Fasting Insulin Adj | MAGIC | Std Dev |
| | | | | | | | | | BMI | | |
| rs2972146 | LOC646736 | 2 | T | G | 0.65 | −0.030 | 0.004 | 1.24E−11 | Body Fat Percentage | UK | Std Dev |
| | | | | | | | | | | Biobank | |
| rs2972146 | LOC646736 | 2 | T | G | 0.65 | 0.007 | 0.004 | 0.100 | Waist Hip Ratio Adj | UK | Std Dev |
| | | | | | | | | | BMI | Biobank | |
| rs2972146 | LOC646736 | 2 | T | G | 0.65 | 0.002 | 0.003 | 0.424 | Height | GIANT | Std Dev |
| rs2972146 | LOC646736 | 2 | T | G | 0.65 | −0.040 | 0.008 | 2.26E−06 | Adiponectin | ADIPOGen | Std Dev |
| rs2972146 | LOC646736 | 2 | T | G | 0.65 | 0.010 | 0.009 | 0.230 | Insulin Secretion | MAGIC | Std Dev |
| rs2972146 | LOC646736 | 2 | T | G | 0.65 | 0.006 | 0.003 | 0.074 | Low Density | GLGC | Std Dev |
| | | | | | | | | | Lipoprotein | | |
| | | | | | | | | | Cholesterol | | |
| rs2972146 | LOC646736 | 2 | T | G | 0.65 | −0.010 | 0.017 | 0.562 | Inflammatory Bowel | IIBDGC | ln(OR) |
| | | | | | | | | | Disease | | |
| rs2972146 | LOC646736 | 2 | T | G | 0.65 | 0.010 | 0.008 | 0.226 | eGFRcys | CKDGen | mL/min/ |
| | | | | | | | | | | | 1.73 m2 |
| rs2972146 | LOC646736 | 2 | T | G | 0.65 | 0.001 | 0.003 | 0.781 | Total Cholesterol | GLGC | Std Dev |
| rs2972146 | LOC646736 | 2 | T | G | 0.65 | 0.077 | 0.019 | 4.68E−05 | Type 2 Diabetes | DIAGAM | ln(OR) |
| rs2972146 | LOC646736 | 2 | T | G | 0.65 | −0.031 | 0.003 | 2.73E−20 | High Density | GLGC | Std Dev |
| | | | | | | | | | Lipoprotein | | |
| | | | | | | | | | Cholesterol | | |
| rs2972146 | LOC646736 | 2 | T | G | 0.65 | 0.028 | 0.003 | 1.41E−16 | Triglycerides | GLGC | Std Dev |
| rs2972146 | LOC646736 | 2 | T | G | 0.65 | −0.002 | 0.004 | 0.664 | eGFRcrea | CKDGen | mL/min/ |
| | | | | | | | | | | | 1.73 m2 |
| rs2972146 | LOC646736 | 2 | T | G | 0.65 | −0.040 | 0.027 | 0.138 | Body Mass Index | GIANT | ln(OR) |
| rs2972146 | LOC646736 | 2 | T | G | 0.65 | 0.128 | 0.086 | 0.137 | Systolic BP | UK | mmHg |
| | | | | | | | | | | Biobank | |
| rs2972146 | LOC646736 | 2 | T | G | 0.65 | 0.059 | 0.048 | 0.220 | Diastolic BP | UK | mmHg |
| | | | | | | | | | | Biobank | |
| rs2972146 | LOC646736 | 2 | T | G | 0.65 | 0.019 | 0.058 | 0.742 | Peripheral Vascular | UK | ln(OR) |
| | | | | | | | | | Disease | Biobank | |
| rs2972146 | LOC646736 | 2 | T | G | 0.65 | 0.093 | 0.039 | 0.017 | Gout | UK | ln(OR) |
| | | | | | | | | | | Biobank | |
| rs2972146 | LOC646736 | 2 | T | G | 0.65 | −0.017 | 0.028 | 0.531 | Migraine | UK | ln(OR) |
| | | | | | | | | | | Biobank | |
| rs2972146 | LOC646736 | 2 | T | G | 0.65 | −0.002 | 0.032 | 0.951 | COPD | UK | ln(OR) |
| | | | | | | | | | | Biobank | |
| rs2972146 | LOC646736 | 2 | T | G | 0.65 | −0.247 | 0.135 | 0.068 | Lung Cancer | UK | ln(OR) |
| | | | | | | | | | | Biobank | |
| rs2972146 | LOC646736 | 2 | T | G | 0.65 | −0.058 | 0.032 | 0.069 | Breast Cancer | UK | ln(OR) |
| | | | | | | | | | | Biobank | |
| rs2972146 | LOC646736 | 2 | T | G | 0.65 | 0.019 | 0.062 | 0.764 | Colorectal Cancer | UK | ln(OR) |
| | | | | | | | | | | Biobank | |
| rs2972146 | LOC646736 | 2 | T | G | 0.65 | −0.035 | 0.016 | 0.030 | Any Cancer | UK | ln(OR) |
| | | | | | | | | | | Biobank | |
| rs17843797 | UMPS- | 3 | G | T | 0.13 | −0.001 | 0.006 | 0.853 | Fasting Insulin Adj | MAGIC | Std Dev |
| | ITGB5 | | | | | | | | BMI | | |
| rs17843797 | UMPS- | 3 | G | T | 0.13 | 0.029 | 0.006 | 2.94E−06 | Body Fat Percentage | UK | Std Dev |
| | ITGB5 | | | | | | | | | Biobank | |
| rs17843797 | UMPS- | 3 | G | T | 0.13 | −0.013 | 0.006 | 0.037 | Waist Hip Ratio Adj | UK | Std Dev |
| | ITGB5 | | | | | | | | BMI | Biobank | |
| rs17843797 | UMPS- | 3 | G | T | 0.13 | 0.011 | 0.004 | 0.009 | Height | GIANT | Std Dev |
| | ITGB5 | | | | | | | | | | |
| rs17843797 | UMPS- | 3 | G | T | 0.13 | −0.007 | 0.013 | 0.579 | Adiponectin | ADIPOGen | Std Dev |
| | ITGB5 | | | | | | | | | | |
| rs17843797 | UMPS- | 3 | G | T | 0.13 | 0.008 | 0.013 | 0.547 | Insulin Secretion | MAGIC | Std Dev |
| | ITGB5 | | | | | | | | | | |
| rs17843797 | UMPS- | 3 | G | T | 0.13 | 0.006 | 0.007 | 0.357 | Low Density | GLGC | Std Dev |
| | ITGB5 | | | | | | | | Lipoprotein | | |
| | | | | | | | | | Cholesterol | | |
| rs17843797 | UMPS- | 3 | G | T | 0.13 | −0.026 | 0.025 | 0.300 | Inflammatory Bowel | IIBDGC | ln(OR) |
| | ITGB5 | | | | | | | | Disease | | |
| rs17843797 | UMPS- | 3 | G | T | 0.13 | −0.029 | 0.012 | 0.015 | eGFRcys | CKDGen | mL/min/ |
| | ITGB5 | | | | | | | | | | 1.73 m2 |
| rs17843797 | UMPS- | 3 | G | T | 0.13 | −0.001 | 0.006 | 0.845 | Total Cholesterol | GLGC | Std Dev |
| | ITGB5 | | | | | | | | | | |
| rs17843797 | UMPS- | 3 | G | T | 0.13 | −0.014 | 0.023 | 0.530 | Type 2 Diabetes | DIAGRAM | ln(OR) |
| | ITGB5 | | | | | | | | | | |
| rs17843797 | UMPS- | 3 | G | T | 0.13 | −0.007 | 0.006 | 0.255 | High Density | GLGC | Std Dev |
| | ITGB5 | | | | | | | | Lipoprotein | | |
| | | | | | | | | | Cholesterol | | |
| rs17843797 | UMPS- | 3 | G | T | 0.13 | 0.005 | 0.007 | 0.429 | Triglycerides | GLGC | Std Dev |
| | ITGB5 | | | | | | | | | | |
| rs17843797 | UMPS- | 3 | G | T | 0.13 | −0.012 | 0.006 | 0.028 | eGFRcrea | CKDGen | mL/min/ |
| | ITGB5 | | | | | | | | | | 1.73 m2 |
| rs17843797 | UMPS- | 3 | G | T | 0.13 | −0.059 | 0.044 | 0.181 | Body Mass Index | GIANT | ln(OR) |
| | ITGB5 | | | | | | | | | | |
| rs17843797 | UMPS- | 3 | G | T | 0.13 | 0.251 | 0.122 | 0.040 | Systolic BP | UK | mmHg |
| | ITGB5 | | | | | | | | | Biobank | |
| rs17843797 | UMPS- | 3 | G | T | 0.13 | 0.033 | 0.068 | 0.631 | Diastolic BP | UK | mmHg |
| | ITGB5 | | | | | | | | | Biobank | |
| rs17843797 | UMPS- | 3 | G | T | 0.13 | 0.034 | 0.084 | 0.687 | Peripheral Vascular | UK | ln(OR) |
| | ITGB5 | | | | | | | | Disease | Biobank | |
| rs17843797 | UMPS- | 3 | G | T | 0.13 | 0.001 | 0.056 | 0.985 | Gout | UK | ln(OR) |
| | ITGB5 | | | | | | | | | Biobank | |
| rs17843797 | UMPS- | 3 | G | T | 0.13 | 0.039 | 0.040 | 0.326 | Migraine | UK | ln(OR) |
| | ITGB5 | | | | | | | | | Biobank | |
| rs17843797 | UMPS- | 3 | G | T | 0.13 | 0.073 | 0.045 | 0.109 | COPD | UK | ln(OR) |
| | ITGB5 | | | | | | | | | Biobank | |
| rs17843797 | UMPS- | 3 | G | T | 0.13 | 0.156 | 0.195 | 0.423 | Lung Cancer | UK | ln(OR) |
| | ITGB5 | | | | | | | | | Biobank | |
| rs17843797 | UMPS- | 3 | G | T | 0.13 | 0.059 | 0.046 | 0.203 | Breast Cancer | UK | ln(OR) |
| | ITGB5 | | | | | | | | | Biobank | |
| rs17843797 | UMPS- | 3 | G | T | 0.13 | 0.077 | 0.088 | 0.381 | Colorectal Cancer | UK | ln(OR) |
| | ITGB5 | | | | | | | | | Biobank | |
| rs17843797 | UMPS- | 3 | G | T | 0.13 | 0.006 | 0.024 | 0.806 | Any Cancer | UK | ln(OR) |
| | ITGB5 | | | | | | | | | Biobank | |
| rs748431 | FGD5 | 3 | G | T | 0.36 | 0.005 | 0.006 | 0.391 | Fasting Insulin Adj | MAGIC | Std Dev |
| | | | | | | | | | BMI | | |
| rs748431 | FGD5 | 3 | G | T | 0.36 | −0.002 | 0.004 | 0.601 | Body Fat Percentage | UK | Std Dev |
| | | | | | | | | | | Biobank | |
| rs748431 | FGD5 | 3 | G | T | 0.36 | 0.005 | 0.004 | 0.236 | Waist Hip Ratio Adj | UK | Std Dev |
| | | | | | | | | | BMI | Biobank | |
| rs748431 | FGD5 | 3 | G | T | 0.36 | −0.003 | 0.003 | 0.301 | Height | GIANT | Std Dev |
| rs748431 | FGD5 | 3 | G | T | 0.36 | 0.001 | 0.008 | 0.893 | Adiponectin | ADIPOGen | Std Dev |
| rs748431 | FGD5 | 3 | G | T | 0.36 | −0.002 | 0.009 | 0.830 | Insulin Secretion | MAGIC | Std Dev |
| rs748431 | FGD5 | 3 | G | T | 0.36 | −0.005 | 0.003 | 0.108 | Low Density | GLGC | Std Dev |
| | | | | | | | | | Lipoprotein | | |
| | | | | | | | | | Cholesterol | | |
| rs748431 | FGD5 | 3 | G | T | 0.36 | −0.004 | 0.017 | 0.799 | Inflammatory Bowel | IIBDGC | ln(OR) |
| | | | | | | | | | Disease | | |
| rs748431 | FGD5 | 3 | G | T | 0.36 | 0.010 | 0.008 | 0.250 | eGFRcys | CKDGen | mL/min/ |
| | | | | | | | | | | | 1.73 m2 |
| rs748431 | FGD5 | 3 | G | T | 0.36 | −0.005 | 0.003 | 0.127 | Total Cholesterol | GLGC | Std Dev |
| rs748431 | FGD5 | 3 | G | T | 0.36 | 0.058 | 0.019 | 0.002 | Type 2 Diabetes | DIAGRAM | ln(OR) |
| rs748431 | FGD5 | 3 | G | T | 0.36 | 0.004 | 0.003 | 0.265 | High Density | GLGC | Std Dev |
| | | | | | | | | | Lipoprotein | | |
| | | | | | | | | | Cholesterol | | |
| rs748431 | FGD5 | 3 | G | T | 0.36 | −0.001 | 0.003 | 0.814 | Triglycerides | GLGC | Std Dev |
| rs748431 | FGD5 | 3 | G | T | 0.36 | −0.002 | 0.004 | 0.664 | eGFRcrea | CKDGen | mL/min/ |
| | | | | | | | | | | | 1.73 m2 |
| rs748431 | FGD5 | 3 | G | T | 0.36 | −0.051 | 0.026 | 0.050 | Body Mass Index | GIANT | ln(OR) |
| rs748431 | FGD5 | 3 | G | T | 0.36 | 0.295 | 0.086 | 0.001 | Systolic BP | UK | mmHg |
| | | | | | | | | | | Biobank | |
| rs748431 | FGD5 | 3 | G | T | 0.36 | 0.109 | 0.048 | 0.023 | Diastolic BP | UK | mmHg |
| | | | | | | | | | | Biobank | |
| rs748431 | FGD5 | 3 | G | T | 0.36 | 0.055 | 0.057 | 0.331 | Peripheral Vascular | UK | ln(OR) |
| | | | | | | | | | Disease | Biobank | |
| rs748431 | FGD5 | 3 | G | T | 0.36 | −0.074 | 0.039 | 0.054 | Gout | UK | ln(OR) |
| | | | | | | | | | | Biobank | |
| rs748431 | FGD5 | 3 | G | T | 0.36 | −0.034 | 0.027 | 0.216 | Migraine | UK | ln(OR) |
| | | | | | | | | | | Biobank | |
| rs748431 | FGD5 | 3 | G | T | 0.36 | −0.007 | 0.032 | 0.820 | COPD | UK | ln(OR) |
| | | | | | | | | | | Biobank | |
| rs748431 | FGD5 | 3 | G | T | 0.36 | −0.311 | 0.146 | 0.033 | Lung Cancer | UK | ln(OR) |
| | | | | | | | | | | Biobank | |
| rs748431 | FGD5 | 3 | G | T | 0.36 | −0.044 | 0.032 | 0.172 | Breast Cancer | UK | ln(OR) |
| | | | | | | | | | | Biobank | |
| rs748431 | FGD5 | 3 | G | T | 0.36 | −0.028 | 0.062 | 0.654 | Colorectal Cancer | UK | ln(OR) |
| | | | | | | | | | | Biobank | |
| rs748431 | FGD5 | 3 | G | T | 0.36 | 0.018 | 0.016 | 0.279 | Any Cancer | UK | ln(OR) |
| | | | | | | | | | | Biobank | |
| rs7623687 | RHOA | 3 | A | C | 0.86 | 0.008 | 0.008 | 0.343 | Fasting Insulin Adj | MAGIC | Std Dev |
| | | | | | | | | | BMI | | |
| rs7623687 | RHOA | 3 | A | C | 0.86 | 0.000 | 0.006 | 0.991 | Body Fat Percentage | UK | Std Dev |
| | | | | | | | | | | Biobank | |
| rs7623687 | RHOA | 3 | A | C | 0.86 | −0.017 | 0.006 | 0.006 | Waist Hip Ratio Adi | UK | Std Dev |
| | | | | | | | | | BMI | Biobank | |
| rs7623687 | RHOA | 3 | A | C | 0.86 | −0.010 | 0.004 | 0.011 | Height | GIANT | Std Dev |
| rs7623687 | RHOA | 3 | A | C | 0.86 | 0.000 | 0.004 | 0.983 | Adiponectin | ADIPOGen | Std Dev |
| rs7623687 | RHOA | 3 | A | C | 0.86 | −0.017 | 0.013 | 0.180 | Insulin Secretion | MAGIC | Std Dev |
| rs7623687 | RHOA | 3 | A | C | 0.86 | 0.002 | 0.007 | 0.753 | Low Density | GLGC | Std Dev |
| | | | | | | | | | Lipoprotein | | |
| | | | | | | | | | Cholesterol | | |
| rs7623687 | RHOA | 3 | A | C | 0.86 | −0.115 | 0.024 | 2.30E−06 | Inflammatory Bowel | IIBDGC | ln(OR) |
| | | | | | | | | | Disease | | |
| rs7623687 | RHOA | 3 | A | C | 0.86 | 0.006 | 0.018 | 0.749 | eGFRcys | CKDGen | mL/min/ |
| | | | | | | | | | | | 1.73 m2 |
| rs7623687 | RHOA | 3 | A | C | 0.86 | 0.003 | 0.005 | 0.593 | Total Cholesterol | GLGC | Std Dev |
| rs7623687 | RHOA | 3 | A | C | 0.86 | 0.015 | 0.024 | 0.523 | Type 2 Diabetes | DIAGRAM | ln(OR) |
| rs7623687 | RHOA | 3 | A | C | 0.86 | 0.001 | 0.004 | 0.713 | High Density | GLGC | Std Dev |
| | | | | | | | | | Lipoprotein | | |
| | | | | | | | | | Cholesterol | | |
| rs7623687 | RHOA | 3 | A | C | 0.86 | 0.001 | 0.005 | 0.799 | Triglycerides | GLGC | Std Dev |
| rs7623687 | RHOA | 3 | A | C | 0.86 | −0.010 | 0.005 | 0.064 | eGFRcrea | CKDGen | mL/min/ |
| | | | | | | | | | | | 1.73 m2 |
| rs7623687 | RHOA | 3 | A | C | 0.86 | 0.092 | 0.038 | 0.014 | Body Mass Index | GIANT | ln(OR) |
| rs7623687 | RHOA | 3 | A | C | 0.86 | 0.041 | 0.119 | 0.728 | Systolic BP | UK | mmHg |
| | | | | | | | | | | Biobank | |
| rs7623687 | RHOA | 3 | A | C | 0.86 | 0.000 | 0.067 | 0.997 | Diastolic BP | UK | mmHg |
| | | | | | | | | | | Biobank | |
| rs7623687 | RHOA | 3 | A | C | 0.86 | −0.058 | 0.081 | 0.475 | Peripheral Vascular | UK | ln(OR) |
| | | | | | | | | | Disease | Biobank | |
| rs7623687 | RHOA | 3 | A | C | 0.86 | 0.005 | 0.055 | 0.933 | Gout | UK | ln(OR) |
| | | | | | | | | | | Biobank | |
| rs7623687 | RHOA | 3 | A | C | 0.86 | −0.013 | 0.039 | 0.737 | Migraine | UK | ln(OR) |
| | | | | | | | | | | Biobank | |
| rs7623687 | RHOA | 3 | A | C | 0.86 | 0.057 | 0.046 | 0.219 | COPD | UK | ln(OR) |
| | | | | | | | | | | Biobank | |
| rs7623687 | RHOA | 3 | A | C | 0.86 | −0.039 | 0.197 | 0.845 | Lung Cancer | UK | ln(OR) |
| | | | | | | | | | | Biobank | |
| rs7623687 | RHOA | 3 | A | C | 0.86 | −0.022 | 0.045 | 0.624 | Breast Cancer | UK | ln(OR) |
| | | | | | | | | | | Biobank | |
| rs7623687 | RHOA | 3 | A | C | 0.86 | 0.057 | 0.089 | 0.521 | Colorectal Cancer | UK | ln(OR) |
| | | | | | | | | | | Biobank | |
| rs7623687 | RHOA | 3 | A | C | 0.86 | −0.026 | 0.023 | 0.255 | Any Cancer | UK | ln(OR) |
| | | | | | | | | | | Biobank | |
| rs12493885 | ARHGEF26 | 3 | C | G | 0.85 | 0.016 | 0.009 | 0.079 | Fasting Insulin Adj | MAGIC | Std Dev |
| | | | | | | | | | BMI | | |
| rs12493885 | ARHGEF26 | 3 | C | G | 0.85 | 0.003 | 0.006 | 0.640 | Body Fat Percentage | UK | Std Dev |
| | | | | | | | | | | Biobank | |
| rs12493885 | ARHGEF26 | 3 | C | G | 0.85 | −0.007 | 0.006 | 0.225 | Waist Hip Ratio Adj | UK | Std Dev |
| | | | | | | | | | BMI | Biobank | |
| rs12493885 | ARHGEF26 | 3 | C | G | 0.85 | 0.004 | 0.005 | 0.338 | Height | GIANT | Std Dev |
| rs12493885 | ARHGEF26 | 3 | C | G | 0.85 | 0.005 | 0.014 | 0.734 | Adiponectin | ADIPOGen | Std Dev |
| rs12493885 | ARHGEF26 | 3 | C | G | 0.85 | 0.028 | 0.014 | 0.046 | Insulin Secretion | MAGIC | Std Dev |
| rs12493885 | ARHGEF26 | 3 | C | G | 0.85 | 0.000 | 0.006 | 0.949 | Low Density | GLGC | Std Dev |
| | | | | | | | | | Lipoprotein | | |
| | | | | | | | | | Cholesterol | | |
| rs12493885 | ARHGEF26 | 3 | C | G | 0.85 | −0.007 | 0.025 | 0.773 | Inflammatory Bowel | IIBDGC | ln(OR) |
| | | | | | | | | | Disease | | |
| rs12493885 | ARHGEF26 | 3 | C | G | 0.85 | −0.007 | 0.012 | 0.544 | eGFRcys | CKDGen | mL/min/ |
| | | | | | | | | | | | 1.73 m2 |
| rs12493885 | ARHGEF26 | 3 | C | G | 0.85 | −0.009 | 0.005 | 0.099 | Total Cholesterol | GLGC | Std Dev |
| rs12493885 | ARHGEF26 | 3 | C | G | 0.85 | 0.033 | 0.027 | 0.228 | Type 2 Diabetes | DIAGRAM | ln(OR) |
| rs12493885 | ARHGEF26 | 3 | C | G | 0.85 | −0.014 | 0.006 | 0.013 | High Density | GLGC | Std Dev |
| | | | | | | | | | Lipoprotein | | |
| | | | | | | | | | Cholesterol | | |
| rs12493885 | ARHGEF26 | 3 | C | G | 0.85 | 0.001 | 0.006 | 0.830 | Triglycerides | GLGC | Std Dev |
| rs12493885 | ARHGEF26 | 3 | C | G | 0.85 | −0.019 | 0.006 | 0.001 | eGFRcrea | CKDGen | mL/min/ |
| | | | | | | | | | | | 1.73 m2 |
| rs12493885 | ARHGEF26 | 3 | C | G | 0.85 | 0.023 | 0.051 | 0.652 | Body Mass Index | GIANT | ln(OR) |
| rs12493885 | ARHGEF26 | 3 | C | G | 0.85 | −0.341 | 0.117 | 0.004 | Systolic BP | UK | mmHg |
| | | | | | | | | | | Biobank | |
| rs12493885 | ARHGEF26 | 3 | C | G | 0.85 | −0.228 | 0.065 | 0.0005 | Diastolic BP | UK | mmHg |
| | | | | | | | | | | Biobank | |
| rs12493885 | ARHGEF26 | 3 | C | G | 0.85 | −0.018 | 0.078 | 0.820 | Peripheral Vascular | UK | ln(OR) |
| | | | | | | | | | Disease | Biobank | |
| rs12493885 | ARHGEF26 | 3 | C | G | 0.85 | 0.107 | 0.054 | 0.046 | Gout | UK | ln(OR) |
| | | | | | | | | | | Biobank | |
| rs12493885 | ARHGEF26 | 3 | C | G | 0.85 | 0.019 | 0.038 | 0.612 | Migraine | UK | ln(OR) |
| | | | | | | | | | | Biobank | |
| rs12493885 | ARHGEF26 | 3 | C | G | 0.85 | −0.036 | 0.043 | 0.402 | COPD | UK | ln(OR) |
| | | | | | | | | | | Biobank | |
| rs12493885 | ARHGEF26 | 3 | C | G | 0.85 | −0.064 | 0.185 | 0.729 | Lung Cancer | UK | ln(OR) |
| | | | | | | | | | | Biobank | |
| rs12493885 | ARHGEF26 | 3 | C | G | 0.85 | −0.028 | 0.043 | 0.516 | Breast Cancer | UK | ln(OR) |
| | | | | | | | | | | Biobank | |
| rs12493885 | ARHGEF26 | 3 | C | G | 0.85 | −0.002 | 0.084 | 0.977 | Colorectal Cancer | UK | ln(OR) |
| | | | | | | | | | | Biobank | |
| rs12493885 | ARHGEF26 | 3 | C | G | 0.85 | 0.009 | 0.022 | 0.679 | Any Cancer | UK | ln(OR) |
| | | | | | | | | | | Biobank | |
| rs10857147 | (FGF5) | 4 | T | A | 0.29 | 0.007 | 0.009 | 0.470 | Fasting Insulin Adj | MAGIC | Std Dev |
| | | | | | | | | | BMI | | |
| rs10857147 | (FGF5) | 4 | T | A | 0.29 | −0.010 | 0.005 | 0.028 | Body Fat Percentage | UK | Std Dev |
| | | | | | | | | | | Biobank | |
| rs10857147 | (FGF5) | 4 | T | A | 0.29 | 0.000 | 0.005 | 0.984 | Waist Hip Ratio Adj | UK | Std Dev |
| | | | | | | | | | BMI | Biobank | |
| rs10857147 | (FGF5) | 4 | T | A | 0.29 | 0.007 | 0.004 | 0.056 | Height | GIANT | Std Dev |
| rs10857147 | (FGF5) | 4 | T | A | 0.29 | −0.024 | 0.011 | 0.027 | Adiponectin | ADIPOGen | Std Dev |
| rs10857147 | (FGF5) | 4 | T | A | 0.29 | 0.007 | 0.013 | 0.592 | Insulin Secretion | MAGIC | Std Dev |
| rs10857147 | (FGF5) | 4 | T | A | 0.29 | 0.003 | 0.005 | 0.551 | Low Density | GLGC | Std Dev |
| | | | | | | | | | Lipoprotein | | |
| | | | | | | | | | Cholesterol | | |
| rs10857147 | (FGF5) | 4 | T | A | 0.29 | 0.009 | 0.020 | 0.652 | Inflammatory Bowel | IIBDGC | ln(OR) |
| | | | | | | | | | Disease | | |
| rs10857147 | (FGF5) | 4 | T | A | 0.29 | 0.012 | 0.010 | 0.239 | eGFRcys | CKDGen | mL/min/ |
| | | | | | | | | | | | 1.73 m2 |
| rs10857147 | (FGF5) | 4 | T | A | 0.29 | 0.004 | 0.005 | 0.363 | Total Cholesterol | GLGC | Std Dev |
| rs10857147 | (FGF5) | 4 | T | A | 0.29 | 0.009 | 0.026 | 0.730 | Type 2 Diabetes | DIAGRAM | ln(OR) |
| rs10857147 | (FGF5) | 4 | T | A | 0.29 | 0.012 | 0.005 | 0.023 | High Density | GLGC | Std Dev |
| | | | | | | | | | Lipoprotein | | |
| | | | | | | | | | Cholesterol | | |
| rs10857147 | (FGF5) | 4 | T | A | 0.29 | −0.003 | 0.005 | 0.513 | Triglycerides | GLGC | Std Dev |
| rs10857147 | (FGF5) | 4 | T | A | 0.29 | 0.023 | 0.005 | 2.08E−06 | eGFRcrea | CKDGen | mL/min/ |
| | | | | | | | | | | | 1.73 m2 |
| rs10857147 | (FGF5) | 4 | T | A | 0.29 | −0.005 | 0.027 | 0.863 | Body Mass Index | GIANT | ln(OR) |
| rs10857147 | (FGF5) | 4 | T | A | 0.29 | 0.866 | 0.091 | 1.90E−21 | Systolic BP | UK | mmHg |
| | | | | | | | | | | Biobank | |
| rs10857147 | (FGF5) | 4 | T | A | 0.29 | 0.491 | 0.051 | 4.93E−22 | Diastolic BP | UK | mmHg |
| | | | | | | | | | | Biobank | |
| rs10857147 | (FGF5) | 4 | T | A | 0.29 | −0.087 | 0.065 | 0.179 | Peripheral Vascular | UK | ln(OR) |
| | | | | | | | | | Disease | Biobank | |
| rs10857147 | (FGF5) | 4 | T | A | 0.29 | −0.036 | 0.042 | 0.385 | Gout | UK | ln(OR) |
| | | | | | | | | | | Biobank | |
| rs10857147 | (FGF5) | 4 | T | A | 0.29 | −0.017 | 0.030 | 0.584 | Migraine | UK | ln(OR) |
| | | | | | | | | | | Biobank | |
| rs10857147 | (FGF5) | 4 | T | A | 0.29 | 0.066 | 0.034 | 0.052 | COPD | UK | ln(OR) |
| | | | | | | | | | | Biobank | |
| rs10857147 | (FGF5) | 4 | T | A | 0.29 | −0.089 | 0.157 | 0.571 | Lung Cancer | UK | ln(OR) |
| | | | | | | | | | | Biobank | |
| rs10857147 | (FGF5) | 4 | T | A | 0.29 | −0.014 | 0.035 | 0.694 | Breast Cancer | UK | ln(OR) |
| | | | | | | | | | | Biobank | |
| rs10857147 | (FGF5) | 4 | T | A | 0.29 | 0.024 | 0.067 | 0.714 | Colorectal Cancer | UK | ln(OR) |
| | | | | | | | | | | Biobank | |
| rs10857147 | (FGF5) | 4 | T | A | 0.29 | 0.005 | 0.018 | 0.786 | Any Cancer | UK | ln(OR) |
| | | | | | | | | | | Biobank | |
| rs7678555 | (MAD2L1) | 4 | C | A | 0.29 | −0.001 | 0.011 | 0.925 | Fasting Insulin Adj | MAGIC | Std Dev |
| | | | | | | | | | BMI | | |
| rs7678555 | (MAD2L1) | 4 | C | A | 0.29 | 0.008 | 0.005 | 0.092 | Body Fat Percentage | UK | Std Dev |
| | | | | | | | | | | Biobank | |
| rs7678555 | (MAD2L1) | 4 | C | A | 0.29 | −0.004 | 0.005 | 0.435 | Waist Hip Ratio Adj | UK | Std Dev |
| | | | | | | | | | BMI | Biobank | |
| rs7678555 | (MAD2L1) | 4 | C | A | 0.29 | 0.003 | 0.003 | 0.414 | Height | GIANT | Std Dev |
| rs7678555 | (MAD2L1) | 4 | C | A | 0.29 | 0.007 | 0.007 | 0.308 | Adiponectin | ADIPOGen | Std Dev |
| rs7678555 | (MAD2L1) | 4 | C | A | 0.29 | 0.018 | 0.010 | 0.060 | Insulin Secretion | MAGIC | Std Dev |
| rs7678555 | (MAD2L1) | 4 | C | A | 0.29 | 0.003 | 0.004 | 0.502 | Low Density | GLGC | Std Dev |
| | | | | | | | | | Lipoprotein | | |
| | | | | | | | | | Cholesterol | | |
| rs7678555 | (MAD2L1) | 4 | C | A | 0.29 | −0.001 | 0.019 | 0.962 | Inflammatory Bowel | IIBDGC | ln(OR) |
| | | | | | | | | | Disease | | |
| rs7678555 | (MAD2L1) | 4 | C | A | 0.29 | 0.010 | 0.009 | 0.261 | eGFRcys | CKDGen | mL/min/ |
| | | | | | | | | | | | 1.73 m2 |
| rs7678555 | (MAD2L1) | 4 | C | A | 0.29 | 0.004 | 0.004 | 0.397 | Total Cholesterol | GLGC | Std Dev |
| rs7678555 | (MAD2L1) | 4 | C | A | 0.29 | 0.002 | 0.012 | 0.836 | Type 2 Diabetes | DIAGRAM | ln(OR) |
| rs7678555 | (MAD2L1) | 4 | C | A | 0.29 | −0.005 | 0.004 | 0.207 | High Density | GLGC | Std Dev |
| | | | | | | | | | Lipoprotein | | |
| | | | | | | | | | Cholesterol | | |
| rs7678555 | (MAD2L1) | 4 | C | A | 0.29 | 0.002 | 0.004 | 0.695 | Triglycerides | GLGC | Std Dev |
| rs7678555 | (MAD2L1) | 4 | C | A | 0.29 | 0.008 | 0.004 | 0.070 | eGFRcrea | CKDGen | mL/min/ |
| | | | | | | | | | | | 1.73 m2 |
| rs7678555 | (MAD2L1) | 4 | C | A | 0.29 | −0.037 | 0.030 | 0.216 | Body Mass Index | GIANT | ln(OR) |
| rs7678555 | (MAD2L1) | 4 | C | A | 0.29 | 0.175 | 0.091 | 0.055 | Systolic BP | UK | mmHg |
| | | | | | | | | | | Biobank | |
| rs7678555 | (MAD2L1) | 4 | C | A | 0.29 | 0.046 | 0.051 | 0.366 | Diastolic BP | UK | mmHg |
| | | | | | | | | | | Biobank | |
| rs7678555 | (MAD2L1) | 4 | C | A | 0.29 | 0.115 | 0.062 | 0.063 | Peripheral Vascular | UK | ln(OR) |
| | | | | | | | | | Disease | Biobank | |
| rs7678555 | (MAD2L1) | 4 | C | A | 0.29 | 0.016 | 0.042 | 0.697 | Gout | UK | ln(OR) |
| | | | | | | | | | | Biobank | |
| rs7678555 | (MAD2L1) | 4 | C | A | 0.29 | −0.043 | 0.030 | 0.154 | Migraine | UK | ln(OR) |
| | | | | | | | | | | Biobank | |
| rs7678555 | (MAD2L1) | 4 | C | A | 0.29 | −0.019 | 0.035 | 0.577 | COPD | UK | ln(OR) |
| | | | | | | | | | | Biobank | |
| rs7678555 | (MAD2L1) | 4 | C | A | 0.29 | 0.006 | 0.153 | 0.968 | Lung Cancer | UK | ln(OR) |
| | | | | | | | | | | Biobank | |
| rs7678555 | (MAD2L1) | 4 | C | A | 0.29 | −0.046 | 0.035 | 0.188 | Breast Cancer | UK | ln(OR) |
| | | | | | | | | | | Biobank | |
| rs7678555 | (MAD2L1) | 4 | C | A | 0.29 | −0.105 | 0.068 | 0.126 | Colorectal Cancer | UK | ln(OR) |
| | | | | | | | | | | Biobank | |
| rs7678555 | (MAD2L1) | 4 | C | A | 0.29 | 0.000 | 0.018 | 0.997 | Any Cancer | UK | ln(OR) |
| | | | | | | | | | | Biobank | |
| rs1800449 | LOX | 5 | T | C | 0.17 | −0.013 | 0.009 | 0.157 | Fasting Insulin Adj | MAGIC | Std Dev |
| | | | | | | | | | BMI | | |
| rs1800449 | LOX | 5 | T | C | 0.17 | 0.008 | 0.006 | 0.155 | Body Fat Percentage | UK | Std Dev |
| | | | | | | | | | | Biobank | |
| rs1800449 | LOX | 5 | T | C | 0.17 | 0.007 | 0.006 | 0.199 | Waist Hip Ratio Adj | UK | Std Dev |
| | | | | | | | | | BMI | Biobank | |
| rs1800449 | LOX | 5 | T | C | 0.17 | 0.012 | 0.004 | 0.006 | Height | GIANT | Std Dev |
| rs1800449 | LOX | 5 | T | C | 0.17 | −0.005 | 0.013 | 0.698 | Adiponectin | ADIPOGen | Std Dev |
| rs1800449 | LOX | 5 | T | C | 0.17 | −0.006 | 0.015 | 0.668 | Insulin Secretion | MAGIC | Std Dev |
| rs1800449 | LOX | 5 | T | C | 0.17 | 0.011 | 0.006 | 0.090 | Low Density | GLGC | Std Dev |
| | | | | | | | | | Lipoprotein | | |
| | | | | | | | | | Cholesterol | | |
| rs1800449 | LOX | 5 | T | C | 0.17 | 0.015 | 0.023 | 0.524 | Inflammatory Bowel | IIBDGC | ln(OR) |
| | | | | | | | | | Disease | | |
| rs1800449 | LOX | 5 | T | C | 0.17 | −0.002 | 0.011 | 0.882 | eGFRcys | CKDGen | mL/min/ |
| | | | | | | | | | | | 1.73 m2 |
| rs1800449 | LOX | 5 | T | C | 0.17 | 0.014 | 0.006 | 0.027 | Total Cholesterol | GLGC | Std Dev |
| rs1800449 | LOX | 5 | T | C | 0.17 | 0.071 | 0.025 | 0.004 | Type 2 Diabetes | DIAGRAM | ln(OR) |
| rs1800449 | LOX | 5 | T | C | 0.17 | 0.005 | 0.007 | 0.426 | High Density | GLGC | Std Dev |
| | | | | | | | | | Lipoprotein | | |
| | | | | | | | | | Cholesterol | | |
| rs1800449 | LOX | 5 | T | C | 0.17 | 0.009 | 0.007 | 0.159 | Triglycerides | GLGC | Std Dev |
| rs1800449 | LOX | 5 | T | C | 0.17 | 0.000 | 0.005 | 0.934 | eGFRcrea | CKDGen | mL/min/ |
| | | | | | | | | | | | 1.73 m2 |
| rs1800449 | LOX | 5 | T | C | 0.17 | 0.028 | 0.046 | 0.543 | Body Mass Index | GIANT | ln(OR) |
| rs1800449 | LOX | 5 | T | C | 0.17 | 0.122 | 0.110 | 0.268 | Systolic BP | UK | mmHg |
| | | | | | | | | | | Biobank | |
| rs1800449 | LOX | 5 | T | C | 0.17 | −0.061 | 0.062 | 0.321 | Diastolic BP | UK | mmHg |
| | | | | | | | | | | Biobank | |
| rs1800449 | LOX | 5 | T | C | 0.17 | −0.048 | 0.075 | 0.522 | Peripheral Vascular | UK | ln(OR) |
| | | | | | | | | | Disease | Biobank | |
| rs1800449 | LOX | 5 | T | C | 0.17 | −0.017 | 0.049 | 0.736 | Gout | UK | ln(OR) |
| | | | | | | | | | | Biobank | |
| rs1800449 | LOX | 5 | T | C | 0.17 | −0.006 | 0.035 | 0.871 | Migraine | UK | ln(OR) |
| | | | | | | | | | | Biobank | |
| rs1800449 | LOX | 5 | T | C | 0.17 | 0.015 | 0.040 | 0.714 | COPD | UK | ln(OR) |
| | | | | | | | | | | Biobank | |
| rs1800449 | LOX | 5 | T | C | 0.17 | −0.110 | 0.185 | 0.550 | Lung Cancer | UK | ln(OR) |
| | | | | | | | | | | Biobank | |
| rs1800449 | LOX | 5 | T | C | 0.17 | −0.070 | 0.042 | 0.095 | Breast Cancer | UK | ln(OR) |
| | | | | | | | | | | Biobank | |
| rs1800449 | LOX | 5 | T | C | 0.17 | −0.064 | 0.081 | 0.428 | Colorectal Cancer | UK | ln(OR) |
| | | | | | | | | | | Biobank | |
| rs1800449 | LOX | 5 | T | C | 0.17 | −0.006 | 0.021 | 0.761 | Any Cancer | UK | ln(OR) |
| | | | | | | | | | | Biobank | |
| rs10841443 | RP11- | 12 | G | C | 0.67 | −0.001 | 0.008 | 0.888 | Fasting Insulin Adj | MAGIC | Std Dev |
| | 664H17.1 | | | | | | | | BMI | | |
| rs10841443 | RP11- | 12 | G | C | 0.67 | −0.006 | 0.005 | 0.188 | Body Fat Percentage | UK | Std Dev |
| | 664H17.1 | | | | | | | | | Biobank | |
| rs10841443 | RP11- | 12 | G | C | 0.67 | 0.001 | 0.005 | 0.845 | Waist Hip Ratio Adj | UK | Std Dev |
| | 664H17.1 | | | | | | | | BMI | Biobank | |
| rs10841443 | RP11- | 12 | G | C | 0.67 | −0.001 | 0.003 | 0.763 | Height | GIANT | Std Dev |
| | 664H17.1 | | | | | | | | | | |
| rs10841443 | RP11- | 12 | G | C | 0.67 | −0.006 | 0.095 | 0.948 | Adiponectin | ADIPOGen | Std Dev |
| | 664H17.1 | | | | | | | | | | |
| rs10841443 | RP11- | 12 | G | C | 0.67 | 0.002 | 0.013 | 0.904 | Insulin Secretion | MAGIC | Std Dev |
| | 664H17.1 | | | | | | | | | | |
| rs10841443 | RP11- | 12 | G | C | 0.67 | −0.009 | 0.005 | 0.081 | Low Density | GLGC | Std Dev |
| | 664H17.1 | | | | | | | | Lipoprotein | | |
| | | | | | | | | | Cholesterol | | |
| rs10841443 | RP11- | 12 | G | C | 0.67 | −0.014 | 0.018 | 0.437 | Inflammatory Bowel | IIBDGC | ln(OR) |
| | 664H17.1 | | | | | | | | Disease | | |
| rs10841443 | RP11- | 12 | G | C | 0.67 | 0.008 | 0.009 | 0.366 | eGFRcys | CKDGen | mL/min/ |
| | 664H17.1 | | | | | | | | | | 1.73 m2 |
| rs10841443 | RP11- | 12 | G | C | 0.67 | −0.005 | 0.005 | 0.246 | Total Cholesterol | GLGC | Std Dev |
| | 664H17.1 | | | | | | | | | | |
| rs10841443 | RP11- | 12 | G | C | 0.67 | 0.005 | 0.025 | 0.846 | Type 2 Diabetes | DIAGRAM | ln(OR) |
| | 664H17.1 | | | | | | | | | | |
| rs10841443 | RP11- | 12 | G | C | 0.67 | −0.007 | 0.005 | 0.159 | High Density | GLGC | Std Dev |
| | 664H17.1 | | | | | | | | Lipoprotein | | |
| | | | | | | | | | Cholesterol | | |
| rs10841443 | RP11- | 12 | G | C | 0.67 | 0.008 | 0.005 | 0.135 | Triglycerides | GLGC | Std Dev |
| | 664H17.1 | | | | | | | | | | |
| rs10841443 | RP11- | 12 | G | C | 0.67 | 0.007 | 0.005 | 0.143 | eGFRcrea | CKDGen | mL/min/ |
| | 664H17.1 | | | | | | | | | | 1.73 m2 |
| rs10841443 | RP11- | 12 | G | C | 0.67 | −0.020 | 0.028 | 0.482 | Body Mass Index | GIANT | ln(OR) |
| | 664H17.1 | | | | | | | | | | |
| rs10841443 | RP11- | 12 | G | C | 0.67 | 0.138 | 0.089 | 0.122 | Systolic BP | UK | mmHg |
| | 664H17.1 | | | | | | | | | Biobank | |
| rs10841443 | RP11- | 12 | G | C | 0.67 | 0.270 | 0.050 | 5.89E−08 | Diastolic BP | UK | mmHg |
| | 664H17.1 | | | | | | | | | Biobank | |
| rs10841443 | RP11- | 12 | G | C | 0.67 | 0.022 | 0.061 | 0.724 | Peripheral Vascular | UK | ln(OR) |
| | 64H17.1 | | | | | | | | Disease | Biobank | |
| rs10841443 | RP11- | 12 | G | C | 0.67 | −0.064 | 0.040 | 0.110 | Gout | UK | ln(OR) |
| | 664H17.1 | | | | | | | | | Biobank | |
| rs10841443 | RP11- | 12 | G | C | 0.67 | −0.008 | 0.029 | 0.795 | Migraine | UK | ln(OR) |
| | 664H17.1 | | | | | | | | | Biobank | |
| rs10841443 | RP11- | 12 | G | C | 0.67 | −0.005 | 0.033 | 0.892 | COPD | UK | ln(OR) |
| | 664H17.1 | | | | | | | | | Biobank | |
| rs10841443 | RP11- | 12 | G | C | 0.67 | 0.071 | 0.150 | 0.638 | Lung Cancer | UK | ln(OR) |
| | 664H17.1 | | | | | | | | | Biobank | |
| rs10841443 | RP11- | 12 | G | C | 0.67 | 0.051 | 0.034 | 0.134 | Breast Cancer | UK | ln(OR) |
| | 664H17.1 | | | | | | | | | Biobank | |
| rs10841443 | RP11- | 12 | G | C | 0.67 | −0.008 | 0.065 | 0.905 | Colorectal Cancer | UK | ln(OR) |
| | 664H17.1 | | | | | | | | | Biobank | |
| rs10841443 | RP11- | 12 | G | C | 0.67 | 0.005 | 0.017 | 0.753 | Any Cancer | UK | ln(OR) |
| | 664H17.1 | | | | | | | | | Biobank | |
| rs2244608 | HNF1A | 12 | G | A | 0.32 | −0.016 | 0.006 | 0.010 | Fasting Insulin Adj | MAGIC | Std Dev |
| | | | | | | | | | BMI | | |
| rs2244608 | HNF1A | 12 | G | A | 0.32 | −0.001 | 0.005 | 0.871 | Body Fat Percentage | UK | Std Dev |
| | | | | | | | | | | Biobank | |
| rs2244608 | HNF1A | 12 | G | A | 0.32 | 0.006 | 0.005 | 0.173 | Waist Hip Ratio Adj | UK | Std Dev |
| | | | | | | | | | BMI | Biobank | |
| rs2244608 | HNF1A | 12 | G | A | 0.32 | 0.003 | 0.003 | 0.399 | Height | GIANT | Std Dev |
| rs2244608 | HNF1A | 12 | G | A | 0.32 | −0.004 | 0.009 | 0.666 | Adiponectin | ADIPOGen | Std Dev |
| rs2244608 | HNF1A | 12 | G | A | 0.32 | −0.025 | 0.009 | 0.005 | Insulin Secretion | MAGIC | Std Dev |
| rs2244608 | HNF1A | 12 | G | A | 0.32 | 0.032 | 0.004 | 2.11E−20 | Low Density | GLGC | Std Dev |
| | | | | | | | | | Lipoprotein | | |
| | | | | | | | | | Cholesterol | | |
| rs2244608 | HNF1A | 12 | G | A | 0.32 | 0.030 | 0.018 | 0.102 | Inflammatory Bowel | IIBDGC | ln(OR) |
| | | | | | | | | | Disease | | |
| rs2244608 | HNF1A | 12 | G | A | 0.32 | −0.018 | 0.008 | 0.032 | eGFRcys | CKDGen | mL/min/ |
| | | | | | | | | | | | 1.73 m2 |
| rs2244608 | HNF1A | 12 | G | A | 0.32 | 0.028 | 0.003 | 2.71E−17 | Total Cholesterol | GLGC | Std Dev |
| rs2244608 | HNF1A | 12 | G | A | 0.32 | 0.058 | 0.019 | 0.002 | Type 2 Diabetes | DIAGRAM | ln(OR) |
| rs2244608 | HNF1A | 12 | G | A | 0.32 | 0.012 | 0.003 | 0.0003 | High Density | GLGC | Std Dev |
| | | | | | | | | | Lipoprotein | | |
| | | | | | | | | | Cholesterol | | |
| rs2244608 | HNF1A | 12 | G | A | 0.32 | 0.001 | 0.003 | 0.689 | Triglycerides | GLGC | Std Dev |
| rs2244608 | HNF1A | 12 | G | A | 0.32 | 0.003 | 0.004 | 0.447 | eGFRcrea | CKDGen | mL/min/ |
| | | | | | | | | | | | 1.73 m2 |
| rs2244608 | HNF1A | 12 | G | A | 0.32 | 0.005 | 0.028 | 0.853 | Body Mass Index | GIANT | ln(OR) |
| rs2244608 | HNF1A | 12 | G | A | 0.32 | 0.099 | 0.089 | 0.265 | Systolic BP | UK | mmHg |
| | | | | | | | | | | Biobank | |
| rs2244608 | HNF1A | 12 | G | A | 0.32 | 0.051 | 0.050 | 0.300 | Diastolic BP | UK | mmHg |
| | | | | | | | | | | Biobank | |
| rs2244608 | HNF1A | 12 | G | A | 0.32 | 0.080 | 0.059 | 0.170 | Peripheral Vascular | UK | ln(OR) |
| | | | | | | | | | Disease | Biobank | |
| rs2244608 | HNF1A | 12 | G | A | 0.32 | 0.042 | 0.039 | 0.290 | Gout | UK | ln(OR) |
| | | | | | | | | | | Biobank | |
| rs2244608 | HNF1A | 12 | G | A | 0.32 | 0.009 | 0.028 | 0.757 | Migraine | UK | ln(OR) |
| | | | | | | | | | | Biobank | |
| rs2244608 | HNF1A | 12 | G | A | 0.32 | 0.080 | 0.032 | 0.013 | COPD | UK | ln(OR) |
| | | | | | | | | | | Biobank | |
| rs2244608 | HNF1A | 12 | G | A | 0.32 | 0.270 | 0.138 | 0.050 | Lung Cancer | UK | ln(OR) |
| | | | | | | | | | | Biobank | |
| rs2244608 | HNF1A | 12 | G | A | 0.32 | 0.032 | 0.033 | 0.333 | Breast Cancer | UK | ln(OR) |
| | | | | | | | | | | Biobank | |
| rs2244608 | HNF1A | 12 | G | A | 0.32 | 0.007 | 0.064 | 0.910 | Colorectal Cancer | UK | ln(OR) |
| | | | | | | | | | | Biobank | |
| rs2244608 | HNF1A | 12 | G | A | 0.32 | 0.019 | 0.017 | 0.270 | Any Cancer | UK | ln(OR) |
| | | | | | | | | | | Biobank | |
| rs11057401 | CCDC92 | 12 | T | A | 0.69 | 0.014 | 0.006 | 0.027 | Fasting Insulin Adj | MAGIC | Std Dev |
| | | | | | | | | | BMI | | |
| rs11057401 | CCDC92 | 12 | T | A | 0.69 | −0.027 | 0.005 | 2.22E−09 | Body Fat Percentage | UK | Std Dev |
| | | | | | | | | | | Biobank | |
| rs11057401 | CCDC92 | 12 | T | A | 0.69 | 0.036 | 0.005 | 1.21E−15 | Waist Hip Ratio Adj | UK | Std Dev |
| | | | | | | | | | BMI | Biobank | |
| rs11057401 | CCDC92 | 12 | T | A | 0.69 | 0.008 | 0.003 | 0.010 | Height | GIANT | Std Dev |
| rs11057401 | CCDC92 | 12 | T | A | 0.69 | −0.052 | 0.009 | 2.24E−09 | Adiponectin | ADIPOGen | Std Dev |
| rs11057401 | CCDC92 | 12 | T | A | 0.69 | 0.018 | 0.009 | 0.046 | Insulin Secretion | MAGIC | Std Dev |
| rs11057401 | CCDC92 | 12 | T | A | 0.69 | 0.015 | 0.005 | 0.002 | Low Density | GLGC | Std Dev |
| | | | | | | | | | Lipoprotein | | |
| | | | | | | | | | Cholesterol | | |
| rs11057401 | CCDC92 | 12 | T | A | 0.69 | 0.057 | 0.018 | 0.002 | Inflammatory Bowel | IIBDGC | ln(OR) |
| | | | | | | | | | Disease | | |
| rs11057401 | CCDC92 | 12 | T | A | 0.69 | −0.006 | 0.008 | 0.453 | eGFRcys | CKDGen | mL/min/ |
| | | | | | | | | | | | 1.73 m2 |
| rs11057401 | CCDC92 | 12 | T | A | 0.69 | 0.015 | 0.005 | 0.003 | Total Cholesterol | GLGC | Std Dev |
| rs11057401 | CCDC92 | 12 | T | A | 0.69 | 0.039 | 0.020 | 0.046 | Type 2 Diabetes | DIAGRAM | ln(OR) |
| rs11057401 | CCDC92 | 12 | T | A | 0.69 | −0.028 | 0.005 | 1.03E−08 | High Density | GLGC | Std Dev |
| | | | | | | | | | Lipoprotein | | |
| | | | | | | | | | Cholesterol | | |
| rs11057401 | CCDC92 | 12 | T | A | 0.69 | 0.027 | 0.005 | 6.64E−08 | Triglycerides | GLGC | Std Dev |
| rs11057401 | CCDC92 | 12 | T | A | 0.69 | −0.010 | 0.004 | 0.012 | eGFRcrea | CKDGen | mL/min/ |
| | | | | | | | | | | | 1.73 m2 |
| rs11057401 | CCDC92 | 12 | T | A | 0.69 | −0.036 | 0.028 | 0.199 | Body Mass Index | GIANT | ln(OR) |
| rs11057401 | CCDC92 | 12 | T | A | 0.69 | −0.128 | 0.089 | 0.149 | Systolic BP | UK | mmHg |
| | | | | | | | | | | Biobank | |
| rs11057401 | CCDC92 | 12 | T | A | 0.69 | −0.080 | 0.050 | 0.107 | Diastolic BP | UK | mmHg |
| | | | | | | | | | | Biobank | |
| rs11057401 | CCDC92 | 12 | T | A | 0.69 | 0.111 | 0.061 | 0.068 | Peripheral Vascular | UK | ln(OR) |
| | | | | | | | | | Disease | Biobank | |
| rs11057401 | CCDC92 | 12 | T | A | 0.69 | 0.025 | 0.040 | 0.533 | Gout | UK | ln(OR) |
| | | | | | | | | | | Biobank | |
| rs11057401 | CCDC92 | 12 | T | A | 0.69 | −0.009 | 0.028 | 0.754 | Migraine | UK | ln(OR) |
| | | | | | | | | | | Biobank | |
| rs11057401 | CCDC92 | 12 | T | A | 0.69 | −0.005 | 0.033 | 0.874 | COPD | UK | ln(OR) |
| | | | | | | | | | | Biobank | |
| rs11057401 | CCDC92 | 12 | T | A | 0.69 | 0.090 | 0.146 | 0.539 | Lung Cancer | UK | ln(OR) |
| | | | | | | | | | | Biobank | |
| rs11057401 | CCDC92 | 12 | T | A | 0.69 | −0.043 | 0.033 | 0.191 | Breast Cancer | UK | ln(OR) |
| | | | | | | | | | | Biobank | |
| rs11057401 | CCDC92 | 12 | T | A | 0.69 | 0.168 | 0.066 | 0.011 | Colorectal Cancer | UK | ln(OR) |
| | | | | | | | | | | Biobank | |
| rs11057401 | CCDC92 | 12 | T | A | 0.69 | −0.005 | 0.017 | 0.770 | Any Cancer | UK | ln(OR) |
| | | | | | | | | | | Biobank | |
| rs3851738 | CFDP1 | 16 | C | G | 0.6 | −0.003 | 0.010 | 0.782 | Fasting Insulin Adj | MAGIC | Std Dev |
| | | | | | | | | | BMI | | |
| rs3851738 | CFDP1 | 16 | C | G | 0.6 | 0.001 | 0.004 | 0.772 | Body Fat Percentage | UK | Std Dev |
| | | | | | | | | | | Biobank | |
| rs3851738 | CFDP1 | 16 | C | G | 0.6 | 0.000 | 0.004 | 0.928 | Waist Hip Ratio Adj | UK | Std Dev |
| | | | | | | | | | BMI | Biobank | |
| rs3851738 | CFDP1 | 16 | C | G | 0.6 | 0.016 | 0.003 | 1.80E−07 | Height | GIANT | Std Dev |
| rs3851738 | CFDP1 | 16 | C | G | 0.6 | −0.009 | 0.009 | 0.293 | Adiponectin | ADIPOGen | Std Dev |
| rs3851738 | CFDP1 | 16 | C | G | 0.6 | 0.006 | 0.009 | 0.501 | Insulin Secretion | MAGIC | Std Dev |
| rs3851738 | CFDP1 | 16 | C | G | 0.6 | −0.009 | 0.005 | 0.070 | Low Density | GLGC | Std Dev |
| | | | | | | | | | Lipoprotein | | |
| | | | | | | | | | Cholesterol | | |
| rs3851738 | CFDP1 | 16 | C | G | 0.6 | −0.056 | 0.017 | 0.001 | Inflammatory Bowel | IIBDGC | ln(OR) |
| | | | | | | | | | Disease | | |
| rs3851738 | CFDP1 | 16 | C | G | 0.6 | −0.001 | 0.007 | 0.845 | eGFRcys | CKDGen | mL/min/ |
| | | | | | | | | | | | 1.73 m2 |
| rs3851738 | CFDP1 | 16 | C | G | 0.6 | −0.006 | 0.005 | 0.212 | Total Cholesterol | GLGC | Std Dev |
| rs3851738 | CFDP1 | 16 | C | G | 0.6 | 0.011 | 0.018 | 0.543 | Type 2 Diabetes | DIAGRAM | ln(OR) |
| rs3851738 | CFDP1 | 16 | C | G | 0.6 | 0.002 | 0.005 | 0.752 | High Density | GLGC | Std Dev |
| | | | | | | | | | Lipoprotein | | |
| | | | | | | | | | Cholesterol | | |
| rs3851738 | CFDP1 | 16 | C | G | 0.6 | −0.007 | 0.005 | 0.175 | Triglycerides | GLGC | Std Dev |
| rs3851738 | CFDP1 | 16 | C | G | 0.6 | 0.008 | 0.004 | 0.059 | eGFRcrea | CKDGen | mL/min/ |
| | | | | | | | | | | | 1.73 m2 |
| rs3851738 | CFDP1 | 16 | C | G | 0.6 | −0.042 | 0.026 | 0.103 | Body Mass Index | GIANT | ln(OR) |
| rs3851738 | CFDP1 | 16 | C | G | 0.6 | 0.414 | 0.084 | 8.08E−07 | Systolic BP | UK | mmHg |
| | | | | | | | | | | Biobank | |
| rs3851738 | CFDP1 | 16 | C | G | 0.6 | 0.116 | 0.047 | 0.013 | Diastolic BP | UK | mmHg |
| | | | | | | | | | | Biobank | |
| rs3851738 | CFDP1 | 16 | C | G | 0.6 | 0.077 | 0.059 | 0.192 | Peripheral Vascular | UK | ln(OR) |
| | | | | | | | | | Disease | Biobank | |
| rs3851738 | CFDP1 | 16 | C | G | 0.6 | 0.041 | 0.039 | 0.293 | Gout | UK | ln(OR) |
| | | | | | | | | | | Biobank | |
| rs3851738 | CFDP1 | 16 | C | G | 0.6 | 0.001 | 0.028 | 0.974 | Migraine | UK | ln(OR) |
| | | | | | | | | | | Biobank | |
| rs3851738 | CFDP1 | 16 | C | G | 0.6 | 0.051 | 0.032 | 0.111 | COPD | UK | ln(OR) |
| | | | | | | | | | | Biobank | |
| rs3851738 | CFDP1 | 16 | C | G | 0.6 | −0.124 | 0.140 | 0.378 | Lung Cancer | UK | ln(OR) |
| | | | | | | | | | | Biobank | |
| rs3851738 | CFDP1 | 16 | C | G | 0.6 | −0.028 | 0.032 | 0.386 | Breast Cancer | UK | ln(OR) |
| | | | | | | | | | | Biobank | |
| rs3851738 | CFDP1 | 16 | C | G | 0.6 | −0.198 | 0.061 | 0.001 | Colorectal Cancer | UK | ln(OR) |
| | | | | | | | | | | Biobank | |
| rs3851738 | CFDP1 | 16 | C | G | 0.6 | −0.018 | 0.017 | 0.288 | Any Cancer | UK | ln(OR) |
| | | | | | | | | | | Biobank | |
| rs7500448 | CDH13 | 16 | A | G | 0.75 | 0.000 | 0.007 | 0.953 | Fasting Insulin Adj | MAGIC | Std Dev |
| | | | | | | | | | BMI | | |
| rs7500448 | CDH13 | 16 | A | G | 0.75 | −0.001 | 0.005 | 0.909 | Body Fat Percentage | UK | Std Dev |
| | | | | | | | | | | Biobank | |
| rs7500448 | CDH13 | 16 | A | G | 0.75 | 0.012 | 0.005 | 0.013 | Waist Hip Ratio Adj | UK | Std Dev |
| | | | | | | | | | BMI | Biobank | |
| rs7500448 | CDH13 | 16 | A | G | 0.75 | 0.005 | 0.003 | 0.127 | Height | GIANT | Std Dev |
| rs7500448 | CDH13 | 16 | A | G | 0.75 | −0.050 | 0.010 | 6.57E−07 | Adiponectin | ADIPOGen | Std Dev |
| rs7500448 | CDH13 | 16 | A | G | 0.75 | 0.006 | 0.010 | 0.532 | Insulin Secretion | MAGIC | Std Dev |
| rs7500448 | CDH13 | 16 | A | G | 0.75 | 0.011 | 0.006 | 0.063 | Low Density | GLGC | Std Dev |
| | | | | | | | | | Lipoprotein | | |
| | | | | | | | | | Cholesterol | | |
| rs7500448 | CDH13 | 16 | A | G | 0.75 | 0.005 | 0.020 | 0.799 | Inflammatory Bowel | IIBDGC | ln(OR) |
| | | | | | | | | | Disease | | |
| rs7500448 | CDH13 | 16 | A | G | 0.75 | 0.002 | 0.010 | 0.794 | eGFRcys | CKDGen | mL/min/ |
| | | | | | | | | | | | 1.73 m2 |
| rs7500448 | CDH13 | 16 | A | G | 0.75 | 0.012 | 0.006 | 0.027 | Total Cholesterol | GLGC | Std Dev |
| rs7500448 | CDH13 | 16 | A | G | 0.75 | −0.039 | 0.022 | 0.074 | Type 2 Diabetes | DIAGRAM | ln(OR) |
| rs7500448 | CDH13 | 16 | A | G | 0.75 | 0.006 | 0.006 | 0.262 | High Density | GLGC | Std Dev |
| | | | | | | | | | Lipoprotein | | |
| | | | | | | | | | Cholesterol | | |
| rs7500448 | CDH13 | 16 | A | G | 0.75 | 0.001 | 0.006 | 0.833 | Triglycerides | GLGC | Std Dev |
| rs7500448 | CDH13 | 16 | A | G | 0.75 | −0.006 | 0.004 | 0.194 | eGFRcrea | CKDGen | mL/min/ |
| | | | | | | | | | | | 1.73 m2 |
| rs7500448 | CDH13 | 16 | A | G | 0.75 | 0.045 | 0.033 | 0.173 | Body Mass Index | GIANT | ln(OR) |
| rs7500448 | CDH13 | 16 | A | G | 0.75 | 0.223 | 0.097 | 0.022 | Systolic BP | UK | mmHg |
| | | | | | | | | | | Biobank | |
| rs7500448 | CDH13 | 16 | A | G | 0.75 | −0.198 | 0.054 | 0.0003 | Diastolic BP | UK | mmHg |
| | | | | | | | | | | Biobank | |
| rs7500448 | CDH13 | 16 | A | G | 0.75 | 0.047 | 0.065 | 0.465 | Peripheral Vascular | UK | ln(OR) |
| | | | | | | | | | Disease | Biobank | |
| rs7500448 | CDH13 | 16 | A | G | 0.75 | −0.001 | 0.042 | 0.972 | Gout | UK | ln(OR) |
| | | | | | | | | | | Biobank | |
| rs7500448 | CDH13 | 16 | A | G | 0.75 | 0.041 | 0.031 | 0.178 | Migraine | UK | ln(OR) |
| | | | | | | | | | | Biobank | |
| rs7500448 | CDH13 | 16 | A | G | 0.75 | 0.057 | 0.035 | 0.106 | COPD | UK | ln(OR) |
| | | | | | | | | | | Biobank | |
| rs7500448 | CDH13 | 16 | A | G | 0.75 | −0.019 | 0.153 | 0.901 | Lung Cancer | UK | ln(OR) |
| | | | | | | | | | | Biobank | |
| rs7500448 | CDH13 | 16 | A | G | 0.75 | −0.022 | 0.035 | 0.526 | Breast Cancer | UK | ln(OR) |
| | | | | | | | | | | Biobank | |
| rs7500448 | CDH13 | 16 | A | G | 0.75 | −0.073 | 0.067 | 0.276 | Colorectal Cancer | UK | ln(OR) |
| | | | | | | | | | | Biobank | |
| rs7500448 | CDH13 | 16 | A | G | 0.75 | −0.016 | 0.018 | 0.381 | Any Cancer | UK | ln(OR) |
| | | | | | | | | | | Biobank | |
| rs8108632 | TGFB1 | 19 | T | A | 0.41 | −0.011 | 0.005 | 0.023 | Fasting Insulin Adj | MAGIC | Std Dev |
| | | | | | | | | | BMI | | |
| rs8108632 | TGFB1 | 19 | T | A | 0.41 | 0.004 | 0.004 | 0.349 | Body Fat Percentage | UK | Std Dev |
| | | | | | | | | | | Biobank | |
| rs8108632 | TGFB1 | 19 | T | A | 0.41 | 0.002 | 0.004 | 0.606 | Waist Hip Ratio Adj | UK | Std Dev |
| | | | | | | | | | BMI | Biobank | |
| rs8108632 | TGFB1 | 19 | T | A | 0.41 | 0.004 | 0.002 | 0.103 | Height | GIANT | Std Dev |
| rs8108632 | TGFB1 | 19 | T | A | 0.41 | 0.005 | 0.049 | 0.916 | Adiponectin | ADIPOGen | Std Dev |
| rs8108632 | TGFB1 | 19 | T | A | 0.41 | 0.000 | 0.021 | 0.983 | Insulin Secretion | MAGIC | Std Dev |
| rs8108632 | TGFB1 | 19 | T | A | 0.41 | −0.007 | 0.003 | 0.036 | Low Density | GLGC | Std Dev |
| | | | | | | | | | Lipoprotein | | |
| | | | | | | | | | Cholesterol | | |
| rs8108632 | TGFB1 | 19 | T | A | 0.41 | 0.043 | 0.018 | 0.020 | Inflammatory Bowel | IIBDGC | ln(OR) |
| | | | | | | | | | Disease | | |
| rs8108632 | TGFB1 | 19 | T | A | 0.41 | −0.015 | 0.009 | 0.101 | eGFRcys | CKDGen | mL/min/ |
| | | | | | | | | | | | 1.73 m2 |
| rs8108632 | TGFB1 | 19 | T | A | 0.41 | −0.007 | 0.003 | 0.013 | Total Cholesterol | GLGC | Std Dev |
| rs8108632 | TGFB1 | 19 | T | A | 0.41 | −0.004 | 0.287 | 0.990 | Type 2 Diabetes | DIAGRAM | ln(OR) |
| rs8108632 | TGFB1 | 19 | T | A | 0.41 | −0.006 | 0.003 | 0.077 | High Density | GLGC | Std Dev |
| | | | | | | | | | Lipoprotein | | |
| | | | | | | | | | Cholesterol | | |
| rs8108632 | TGFB1 | 19 | T | A | 0.41 | −0.003 | 0.003 | 0.258 | Triglycerides | GLGC | Std Dev |
| rs8108632 | TGFB1 | 19 | T | A | 0.41 | 0.001 | 0.004 | 0.765 | eGFRcrea | CKDGen | mL/min/ |
| | | | | | | | | | | | 1.73 m2 |
| rs8108632 | TGFB1 | 19 | T | A | 0.41 | −0.007 | 0.029 | 0.805 | Body Mass Index | GIANT | ln(OR) |
| rs8108632 | TGFB1 | 19 | T | A | 0.41 | 0.217 | 0.087 | 0.013 | Systolic BP | UK | mmHg |
| | | | | | | | | | | Biobank | |
| rs8108632 | TGFB1 | 19 | T | A | 0.41 | 0.053 | 0.049 | 0.276 | Diastolic BP | UK | mmHg |
| | | | | | | | | | | Biobank | |
| rs8108632 | TGFB1 | 19 | T | A | 0.41 | 0.023 | 0.058 | 0.698 | Peripheral Vascular | UK | ln(OR) |
| | | | | | | | | | Disease | Biobank | |
| rs8108632 | TGFB1 | 19 | T | A | 0.41 | 0.053 | 0.038 | 0.169 | Gout | UK | ln(OR) |
| | | | | | | | | | | Biobank | |
| rs8108632 | TGFB1 | 19 | T | A | 0.41 | −0.053 | 0.028 | 0.056 | Migraine | UK | ln(OR) |
| | | | | | | | | | | Biobank | |
| rs8108632 | TGFB1 | 19 | T | A | 0.41 | 0.062 | 0.032 | 0.051 | COPD | UK | ln(OR) |
| | | | | | | | | | | Biobank | |
| rs8108632 | TGFB1 | 19 | T | A | 0.41 | 0.104 | 0.141 | 0.461 | Lung Cancer | UK | ln(OR) |
| | | | | | | | | | | Biobank | |
| rs8108632 | TGFB1 | 19 | T | A | 0.41 | 0.011 | 0.032 | 0.730 | Breast Cancer | UK | ln(OR) |
| | | | | | | | | | | Biobank | |
| rs8108632 | TGFB1 | 19 | T | A | 0.41 | 0.023 | 0.062 | 0.715 | Colorectal Cancer | UK | ln(OR) |
| | | | | | | | | | | Biobank | |
| rs8108632 | TGFB1 | 19 | T | A | 0.41 | 0.001 | 0.017 | 0.934 | Any Cancer | UK | ln(OR) |
| | | | | | | | | | | Biobank | |

Bolded phenotypes represent statistically significant pleiotropic associations. Abbreviations: Std Dev, Standard Deviation; OR, Odds Ratio; mmHg, millimeters of mercury; mL, milliliters; min, minutes; BMI, Body Mass Index; BP, Blood Pressure; COPD, Chronic Obstructive Pulmonary Disease; DIAGRAM, DIAbetes Genetics Replication And Meta-analysis; GIANT, Genetic Investigation of ANthropometric Traits; GLGC, Global Lipids Genetics Consortium; MAGIC, Meta-Analyses of Glucose and Insulin-related traits Consortium; CKDGen, Chronic Kidney Disease Genetics Consortium; IIBDGC, International Inflammatory Bowel Disease Genetics Consortium; eGFR, estimated glomerular filtration rate; crea, creatinine; cys, cystatin-c; Chr, chromosome; SE, standard error.

Compelling additional insights from the PheWAS emerged at the CCDC92 locus. Across 25 distinct traits and disorders, Applicants observed significant associations (P<0.00013) for CCDC92 p.Ser70Cys (rs11057401) with body fat percentage, waist-to-hip circumference ratio, as well as plasma high-density lipoprotein, triglyceride, and adiponectin levels. The directionality of these associations are hallmarks of insulin resistance and lipodystrophy (Manning, A. K. et al., Nat Genet 44, 659-69 (2012); Shungin, D. et al., Nature 518, 187-96 (2015)), and the association with plasma adiponectin levels localizes these genetic effects to adipose tissue. Recent work has highlighted two candidate genes at this locus, CCDC92 and DNAH10 (Lotta, L. A. et al., Nat Genet (2016)).

However, a few of the CAD loci (FN1, LOX, ITGB5, and ARHGEF26) did not associate with any of the studied risk factor traits and thus, appear to function through pathways beyond known CAD risk factors (FIG. 2, Tables 6-7). A common variant within an intron of FN1 (Sakai, T., Larsen, M. & Yamada, K. M., Nature 423, 876-81 (2003)) (encoding Fibronectin 1) and a missense variant in LOX (Erler, J. T. et al., Nature 440, 1222-6 (2006)) (encoding Lysyl Oxidase) suggest potential links to extracellular matrix biology. Of note, rare coding mutations in LOX were recently described to cause Mendelian forms of thoracic aortic aneurysm and dissection (Lee, V. S. et al., Proc Natl Acad Sci USA 113, 8759-64 (2016); Guo, D. C. et al., Circ Res 118, 928-34 (2016)), highlighting a potential common link between atherosclerosis and aortic disease, possibly through altered extracellular matrix biology. A variant downstream of ITGB5 (Hood, J. D. & Cheresh, D. A., Nat Rev Cancer 2, 91-100 (2002)) (encoding Integrin Subunit Beta 5) suggests pathways underlying cell adhesion and migration.

In aggregate, the analysis brings the total number of known CAD loci to 95 (Schunkert, H. et al., Nat Genet 43, 333-8 (2011); Deloukas, P. et al., Nat Genet 45, 25-33 (2013); CARDIoGRAMplusC4D Consortium. A comprehensive 1000 Genomes-based genome-wide association meta-analysis of coronary artery disease. Nat Genet 47, 1121-30 (2015); Myocardial Infarction Genetics and CARDIoGRAM Exome Consortia Investigators. Coding Variation in ANGPTL4, LPL, and SVEP1 and the Risk of Coronary Disease. N Engl J Med 374, 1134-44 (2016); Nioi, P. et al., N Engl J Med 374, 2131-41 (2016); Webb, T. R. et al., JAm Coil Cardiol 69, 823-836 (2017); Howson, J. M. M. et al., Nature Genetics (2017)), and in FIG. 3, Applicants organize these loci into plausible pathways. Of note, the causal variant, gene, cell type, and mechanism has been definitively identified at only a few of these loci and as such, additional experimental research will be required, particularly at >50% of loci without an apparent link to known risk factors.

At one of the new loci that did not relate to known risk factors, ARHGEF26 (encoding Rho Guanine Nucleotide Exchange Factor 26), Applicants performed functional studies. Prior experimental work had connected this gene with murine atherosclerosis (Samson, T. et al., PLoS One 8, e55202 (2013)). Earlier studies established a role for ARHGEF26 in facilitating the transendothelial migration of leukocytes, a key step in the initiation of atherosclerosis (van Rijssel, J. et al., Mol Biol Cell 23, 2831-44 (2012); van Buul, J. D. et al., J Cell Biol 178, 1279-93 (2007)). ARHGEF26 has been shown to activate RhoG GTPase by promoting the exchange of GDP by GTP and contributing to the formation of ICAM-1-induced endothelial docking structures that facilitate leukocyte transendothelial migration (van Rijssel, J. et al., Mol Biol Cell 23, 2831-44 (2012); van Buul, J. D. et al., J Cell Biol 178, 1279-93 (2007)). In addition, Arhgef26−/− mice, when crossed with atherosclerosis-prone Apoe null mice, displayed less aortic atherosclerosis (Samson, T. et al., PLoS One 8, e55202 (2013)).

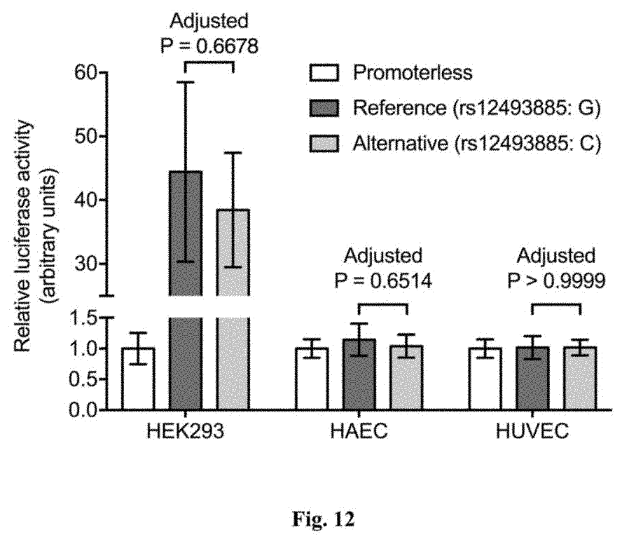

At ARHGEF26 p.Val29Leu (rs12493885), the 29Leu allele, observed in 85% of participants, is associated with increased risk for CAD. Applicants first examined the hypothesis that a haplotype block containing this variant may alter expression of ARHGEF26 in coronary artery. While this region demonstrates eQTL effects in a variety of tissues, there is no evidence of alteration of ARHGEF26 expression in coronary artery in both eQTL and allele specific expression analyses (FIG. 11). To further evaluate the possibility that a haplotype containing the 29Leu allele may affect gene expression, Applicants performed a luciferase reporter assay. Applicants cloned a 2.5 kb region immediately upstream of the ARHGEF26 start codon consisting of the promoter, 5′ untranslated region (5′ UTR), and regions with ENCODE annotations suggestive of potential cis-acting elements. Applicants obtained the reference (in LD with Val29 G allele) and alternative (in LD with 29Leu C allele) haplotypes of this region from human rs12493885 heterozygotes. Applicants coupled each haplotype with a luciferase reporter, and measured luciferase activity (FIG. 12). In HEK293, human aortic endothelial cells (HAEC), and human umbilical vein endothelial cells (HUVEC), there is no significant difference in luciferase activity between reference and alternative haplotypes. These data suggest that the ARHGEF26 29Leu allele may confer CAD risk via mechanisms other than affecting ARHGEF26 transcription or promoter activity in disease-relevant tissue.

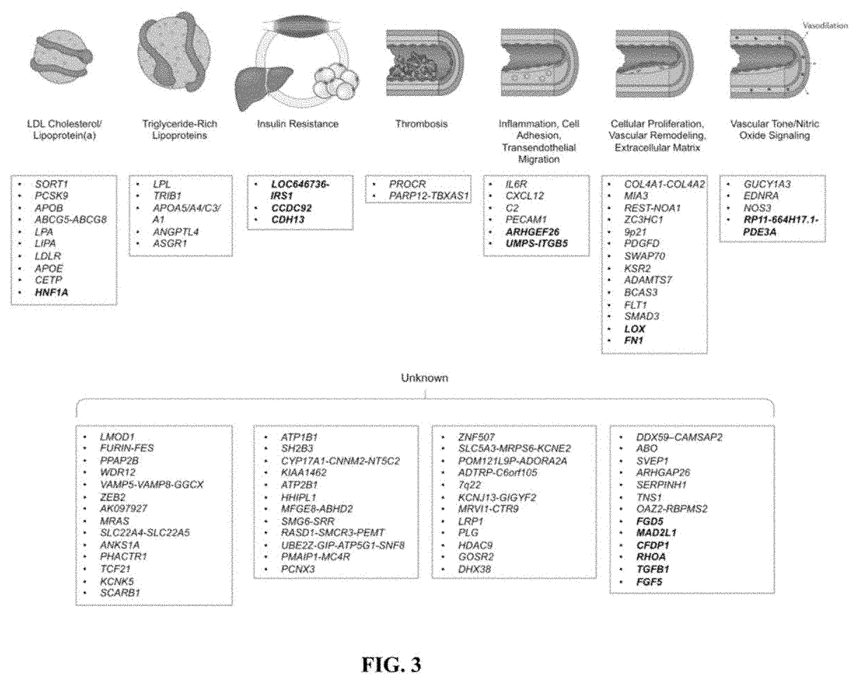

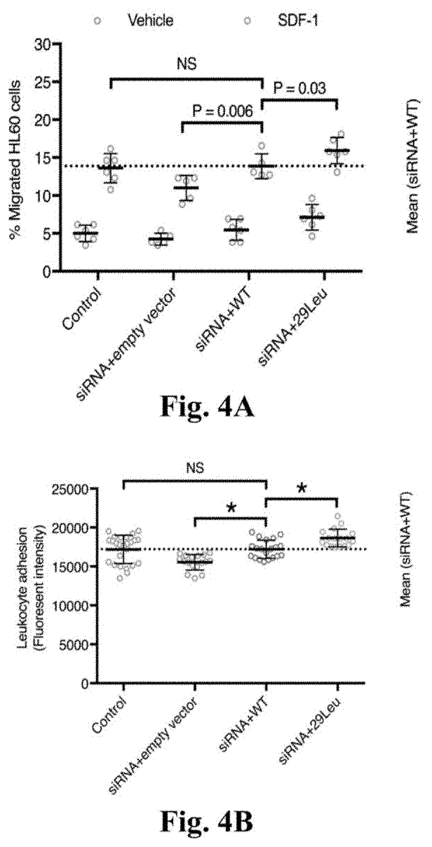

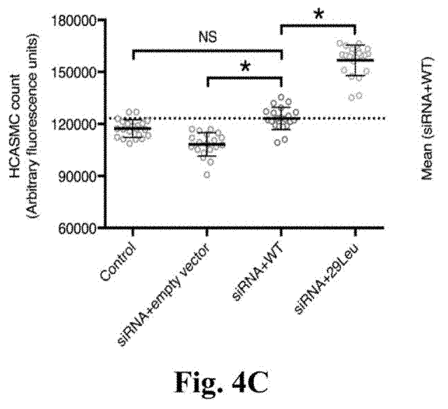

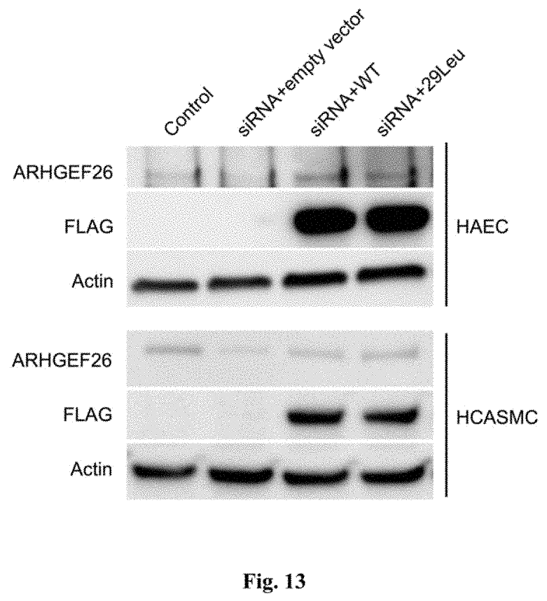

Next, Applicants examined whether ARHGEF26 p.Val29Leu may influence disease risk through its protein-altering consequence. Applicants knocked down endogenous ARHGEF26 through siRNA and observed decreased leukocyte transendothelial migration, leukocyte adhesion on endothelial cells, and vascular smooth cell proliferation (Zahedi, F. et al., Cell Mol Life Sci (2016)) (FIG. 4, FIG. 13). Overexpression of exogenous, wild-type ARHGEF26 rescued these phenotypes. However, ARHGEF26 29Leu mutant overexpression led to rescued phenotypes that consistently exceeded wild-type. These data support that the ARHGEF26 29Leu allele associated with increased CAD risk may lead to a gain-of-function ARHGEF26 protein.

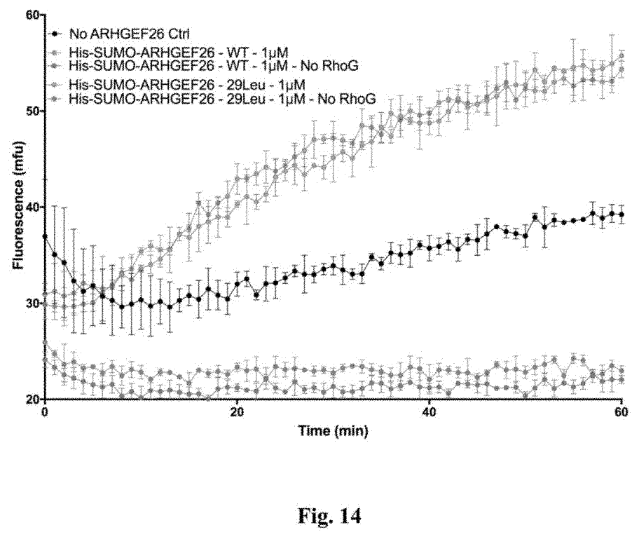

How could the ARHGEF26 29Leu mutation lead to a gain-of-function phenotype? Applicants evaluated its functional impact in two ways, addressing ARHGEF26 quality and quantity, respectively. First, could the 29Leu mutation alter ARHGEF26 nucleotide exchange activity on RhoG? To answer this question, Applicants developed a GTP-GDP nucleotide exchange assay using recombinant human full-length ARHGEF26 (wild-type or 29Leu) and RhoG proteins (Ellerbroek, S. M. et al., Mol Biol Cell 15, 3309-19 (2004)). In a cell-free system, equal amount of wild-type or 29Leu ARHGEF26 protein was incubated with RhoG pre-loaded with GDP. After 60 minutes, Applicants observed no significant difference in nucleotide exchange activity between wild-type and 29Leu mutant ARHGEF26 (FIG. 14).

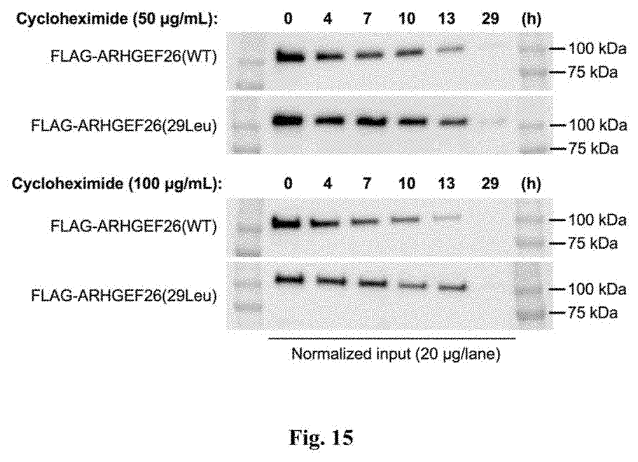

Second, could the 29Leu allele affect cellular abundance of ARHGEF26 protein? Applicants examined this possibility by treating cells expressing wild-type or 29Leu mutant ARHGEF26 with cycloheximide, a protein synthesis inhibitor, and compared ARHGEF26 degradation over time by Western blotting. Compared to wild-type ARHGEF26, the 29Leu mutant protein displayed a longer half-life (FIG. 15). While further work is needed to understand the mechanism in vivo, in vitro results suggest that the gain of function phenotype observed may be secondary to the 29Leu mutant protein's resistance to degradation.

In summary, Applicants performed a gene discovery study for CAD using a large population-based biobank, identified 15 new loci, and explored the phenotypic consequences of CAD risk variants through PheWAS and in vitro functional analysis. These findings permit several conclusions. First, CAD cases phenotyped via electronic health records and verbal interviews exhibit similar genetic architecture to those derived in epidemiologic cohorts and can prove useful in gene discovery efforts. Second, phenome-wide association studies with risk variants can provide initial clues on how DNA sequence variants may lead to disease. Lastly, considerable experimental evidence in cells and rodents has suggested that transendothelial migration of leukocytes is a key step in the formation of atherosclerosis (Gerhardt, T. & Ley, K., Cardiovasc Res 107, 321-30 (2015)); here, Applicants provide human genetic support for a role of this pathway in CAD.

### Study Design and Samples

Applicants performed a three-stage sequential analysis to identify novel genetic loci associated with CAD. In Stage 1, Applicants first tested the association of DNA sequence variants with CAD in UK Biobank. Beginning in 2006, individuals aged 45 to 69 years old were recruited from across the United Kingdom for participation in the UK Biobank Study (Collins, R. What makes UK Biobank special? The Lancet 379, 1173-1174 (2012)). At enrollment, a trained healthcare provider ascertained participants' medical histories through verbal interview. In addition, participants' electronic health records (EHR) including inpatient International Classification of Disease (ICD-10) diagnosis codes and Office of Population and Censuses Surveys (OPCS-4) procedure codes, were integrated into UK Biobank. Individuals were defined as having CAD based on at least one of the following criteria:
1) Myocardial infarction (MI), coronary artery bypass grafting, or coronary artery angioplasty documented in medical history at time of enrollment by a trained nurse
2) Hospitalization for ICD-10 code for acute myocardial infarction (121.0, 121.1, 121.2, 121.4, 121.9)
3) Hospitalization for OPCS-4 coded procedure: coronary artery bypass grafting (K40.1-40.4, K41.1-41.4, K45.1-45.5)
4) Hospitalization for OPCS-4 coded procedure: coronary angioplasty with or without stenting (K49.1-49.2, K49.8-49.9, K50.2, K75.1-75.4, K75.8-75.9)

All other individuals were defined as controls. In total, genotypes were available for 120,286 participants of European ancestry.

In Stage 2, Applicants took forward 2,190 variants that reached nominal significance in Stage 1 for meta-analysis in the Coronary ARtery DIsease (Genome wide Replication and Meta-analysis (CARDIoGRAM) Exome Consortia exome array analysis which incorporated 42,355 cases and 78,240 controls⁶ (Table 8). In Stage 3, Applicants took forward 387,174 variants that reached nominal significance in Stage 1 (and not available in Stage 2) for meta-analysis into the CARDIoGRAMplusC4D 1000 Genomes imputation study containing 60,801 cases and 123,504 controls⁵ (www.cardiogramplusc4d.org/). Informed consent was obtained for all participants, and UK Biobank received ethical approval from the Research Ethics Committee (reference number 11/NW/0382). Our study was approved by a local Institutional Review Board at Partners Healthcare (protocol 2013P001840).

TABLE 8
Table 8 - Sources of cases and controls in the CARDIoGRAM Exome Consortia Study for Stage 2. Samples for this study
were genotyped on the Illumina Human-Exome BeadChip array (version 1.0 or 1.1) or the Illumina OmniExome array.

| Study | Design | Case definition | Control definition | Cases | Controls | Reference |
|---|---|---|---|---|---|---|
| ATVB | Case- | MI in men or women ≤45 years of | No history of | 1,428 | 1,069 | PMID: |
| | control | age | thromboembolic | | | 12615 |
| | | | disease | | | 788 |
| BHF- | Case- | CAD cases were recruited from the | Controls were selected | 2,833 | 5,912 | PMID: |
| FHS | control | British Heart Foundation Family | from the UK 1958 Birth | | | 23202 |
| | | Heart Study and supplemented by | Cohort | | | 125, |
| | | additional cases from WTCCC- | | | | PMID: |
| | | CAD2 | | | | 17634 |
| | | | | | | 449 |
| BioVU | Case- | Cases with MI or CAD were | Controls were | 4,587 | 16,556 | PMID: |
| | control | ascertained from the Vanderbilt | individuals from the | | | 25410 |
| | | University Medical Center | Vanderbilt University | | | 959 |
| | | Biorepository by searching the | Biorepository who did | | | |
| | | electronic medical record for ≥2 | not have any record of | | | |
| | | instances of ICD-9 codes 410.x- | ICD-9 codes 410.x- | | | |
| | | 414.x | 414.x | | | |
| Duke | Case- | MI or coronary stenosis ≥50% | Controls were >50 | 660 | 515 | PMID: |
| | control | | years old without | | | 22319 |
| | | | coronary stenosis >30% | | | 020 |
| | | | and without | | | |
| | | | history of MI, coronary | | | |
| | | | artery bypass grafting, | | | |
| | | | percutaneous coronary | | | |
| | | | intervention, or heart | | | |
| | | | transplant | | | |
| EPIC | Nested | The EPIC (European Prospective | Controls were study | 1,386 | 7,037 | PMID: |
| CAD | case- | Study into Cancer and Nutrition) | participants who | | | 10466 |
| | control | study sub-cohorts from the UK | remained free of any | | | 767 |
| | | were used. Subjects were collected | cardiovascular disease | | | |
| | | in collaboration with general | during follow-up | | | |
| | | practitioners, mainly in | (defined as ICD-9 401- | | | |
| | | Cambridgeshire and Norfolk. | 448 and ICD-10 I10- | | | |
| | | Cases were individuals who | I79) | | | |
| | | developed fatal or non-fatal CAD | | | | |
| | | during an average follow-up of 11 | | | | |
| | | years ending June 2006. | | | | |
| | | Participants were identified if they | | | | |
| | | had a hospital admission and/or | | | | |
| | | died with CAD as the underlying | | | | |
| | | cause. CAD was defined as cause | | | | |
| | | of death codes ICD-9 410-414 or | | | | |
| | | ICD-10 I20-I25, and hospital | | | | |
| | | discharge codes ICD-10 I20.0, I21, | | | | |
| | | I22, or I23 according to the | | | | |
| | | International Classification of | | | | |
| | | Diseases, 9^{th} and 10^{th} revisions, | | | | |
| | | respectively. | | | | |
| FIA3 | Nested | Cases of MI occurring in | Individuals free of MI | 2,473 | 2,047 | PMID: |
| | case- | participants from Vasterbotten | from VIP and MSP | | | 23528 |
| | control | Intervention Program (VIP), | | | | 041, |
| | | WHO's Multinational Monitoring | | | | PMID: |
| | | of Trends and Determinants in | | | | 14660 |
| | | Cardiovascular Disease | | | | 242 |
| | | (MONICA) study in northern | | | | |
| | | Sweden and the Mammography | | | | |
| | | Screening Project (MSP) in | | | | |
| | | Vasterbotten | | | | |
| GoDARTS | Case- | The GoDARTS (Genetics of | Controls were free of | 1,568 | 2,772 | PMID: |
| CAD | control | Diabetes Audit and Research in | CAD, stroke, and | | | 93293 |
| | | Tayside Scotland) study is a joint | peripheral vascular | | | 09 |
| | | initiative of the Department of | disease | | | |
| | | Medicine and the Medicines | | | | |
| | | Monitoring Unit (MEMO) at the | | | | |
| | | University of Dundee, the diabetes | | | | |
| | | units at three Tayside healthcare | | | | |
| | | trusts (Ninewells Hospital and | | | | |
| | | Medical School, Dundee; Perth | | | | |
| | | Royal Infirmary; and Stracathro | | | | |
| | | Hospital, Brechin), and a large | | | | |
| | | group of Tayside general | | | | |
| | | practitioners with an interest in | | | | |
| | | diabetes care. Cases were first-ever | | | | |
| | | CAD event, defined as fatal and | | | | |
| | | non-fatal myocardial infarction, | | | | |
| | | unstable angina, or coronary | | | | |
| | | revascularization. | | | | |
| EGCUT | | CAD or MI cases were ascertained | Controls were selected | 392 | 777 | PMID: |
| | | from the Estonian Biobank | from the Estonian | | | 24518 |
| | | (Estonian Genome Center at the | Biobank (Estonian | | | 929 |
| | | University of Tartu) using the | Genome Center at the | | | |
| | | medical history and current health | University of Tartu) | | | |
| | | status that is recorded according to | who did not have any | | | |
| | | ICD-10 codes (CAD defined with | record of cardiovascular | | | |
| | | ICD-10 I20-I25). | diseases (ICD-10 I10- | | | |
| | | | I79). | | | |
| German CAD | | The German North cohort includes | Controls were derived | 4,464 | 2,886 | PMID: |
| North | | individuals from GerMIFS4, | from population-based | | | 16490 |
| | | PopGen, and HNR with MI or | studies in Germany. | | | 960, |
| | | CAD. | | | | PMID: |
| | | | | | | 12177 |
| | | | | | | 636 |
| German CAD | | The German South cohort includes | Controls were derived | 5,255 | 2,921 | PMID: |
| South | | samples from GerMIFS3 and | from population-based | | | 21088 |
| | | Munich-MI with MI or CAD. | studies in Germany. | | | 011, |
| | | | | | | PMID: |
| | | | | | | 21511 |
| | | | | | | 257 |
| HUNT | Case- | MI Cases were retrospectively | Controls were selected | 2,351 | 2,348 | PMID: |
| | control | identified as HUNT 2 and HUNT 3 | among HUNT 2 and | | | 22879 |
| | | participants diagnosed with acute | HUNT 3 participants | | | 362 |
| | | MI (ICD-10 I21 or ICD-9 410) in | with available DNA | | | |
| | | the medical departments at the two | (N = 70,300) after | | | |
| | | local hospitals in Nord-Trøndelag | excluding individuals | | | |
| | | County from December 1987 to | with the following | | | |
| | | June 2011. | hospital diagnosed or | | | |
| | | | self-reported conditions | | | |
| | | | in themselves or known | | | |
| | | | 1st and/or 2nd degree | | | |
| | | | family members: MI, | | | |
| | | | angina, heart failure, | | | |
| | | | stroke, aortic aneurysm, | | | |
| | | | atherosclerosis, | | | |
| | | | intermittent | | | |
| | | | claudication, and | | | |
| | | | registered percutaneous | | | |
| | | | coronary angioplasty | | | |
| | | | procedures or bypass | | | |
| | | | surgery. | | | |
| BioMe | Case- | CAD cases were ascertained from | Controls were | 704 | 1,729 | NIH |
| Biobank | control | the BioMe Biobank using the | individuals from the | | | dbGaP |
| | | electronic health record with ICD9 | BioMe Biobank who | | | Study |
| | | codes 410.xx to 414.xx and | did not meet the criteria | | | Acces- |
| | | abnormal stress test or abnormal | for cases | | | sion |
| | | coronary angiography | | | | phs000 |
| | | | | | | 388.v1.p1 |
| MDC | Prospective | Prevalent and incident nonfatal or | Participants free of | 2,283 | 4,511 | PMID: |
| | cohort | fatal MI | CHD at baseline and | | | 18354 |
| | | | during follow-up | | | 102 |
| MHI | Case- | Cases were ascertained from the | Controls were | 3,990 | 6,585 | PMID: |
| | control | Montreal Heart Institute Biobank. | individuals from the | | | 24777 |
| | | CAD was defined as the presence | Montreal Heart Institute | | | 453, |
| | | of MI, percutaneous coronary | Biobank who were free | | | PMID: |
| | | intervention, or coronary artery | of history of MI, | | | 25214 |
| | | bypass grafting | percutaneous coronary | | | 527 |
| | | | intervention, or | | | |
| | | | coronary artery bypass | | | |
| | | | grafting | | | |
| OHS | Case- | Cases had angiographically | Asymptomatic males >65, | 1,024 | 2,267 | PMID: |
| | control | confirmed coronary artery disease | females >70 | | | 17478 |
| | | (>1 coronary artery with >50% | | | | 681 |
| | | stenosis) and did not have type 2 | | | | |
| | | diabetes; ≤50 years old for males | | | | |
| | | and ≤50 years old for females | | | | |
| PAS- | Case- | Symptomatic CAD before 51 years | More than 95% of the | 728 | 808 | PMID: |
| AMC | control | of age, defined as MI, coronary | controls are from the | | | 12176 |
| | | revascularization, or evidence of at | same region as cases | | | 944 |
| | | least 70% stenosis in a major | | | | |
| | | epicardial coronary artery | | | | |
| PennCath | Case- | Cases had angiographically | Normal coronary | 683 | 156 | PMID: |
| | control | confirmed coronary artery disease | angiography in men >40 | | | 21239 |
| | | (>1 coronary artery with 50% | years old and | | | 051 |
| | | stenosis); ≤55 years old for males | women >45 years old | | | |
| | | and ≤60 years old for females | | | | |
| PROCARDIS | Case- | Symptomatic CAD before age 66. | No personal or sibling | 2,490 | 2,220 | PMID: |
| | control | CAD was defined as clinically | history of CAD before | | | 20032 |
| | | documented evidence of | age 66 | | | 323 |
| | | myocardial infarction, coronary | | | | |
| | | artery bypass grafting, acute | | | | |
| | | coronary syndrome, coronary | | | | |
| | | angioplasty, or stable angina | | | | |
| VHS | Case- | Documented MI, coronary artery | Normal coronary | 176 | 164 | PMID: |
| | control | bypass grafting, CAD (by | angiography in males >60 | | | 19198 |
| | | angiography) in males ≤45 years | years old or females >65 | | | 609 |
| | | old and females ≤50 years old | years old. | | | |
| WHI | Prospective | Cases were individuals from the | Participants free of | 2,860 | 14,960 | PMID: |
| | cohort | Women's Health Initiative who | CHD on follow-up | | | 94929 |
| | | had incident MI, coronary | | | | 70 |
| | | revascularization, hospitalized | | | | |
| | | angina or death due to coronary | | | | |
| | | disease | | | | |
| Stge 2 | | | | 42,335 | 78,240 | |
| Total | | | | | | |

ATVB: Italian Atherosclerosis, Thrombosis, and Vascular Biology Study; BHF-FHS: British Heart Foundation Family Heart Study; BioVU: Vanderbilt University Medical Center Biorepository; GoDARTS: Genetics of Diabetes Audit and Research Tayside; FIA3: First-time incidence of myocardial infarction in the AC county 3; EGCUT: Estonian Genome Centre, University of Tartu; EPIC: European Prospective Study into Cancer and Nutrition; HUNT: Nord-Trøndelag health study; IPM: Mt. Sinai Institute for Personalized Medicine Biobank; MDC: Malmo Diet and Cancer Study-Cardiovascular Cohort; MHI: Montreal Heart Institute Study; OHS: Ottawa Heart Study; PAS-AMC; Premature Atherosclerosis Study at Academic Medical Center Amsterdam; PennCath: University of Pennsylvania Catheterization Study; PROCARDIS: Precocious Coronary Artery Disease Study; VHS: Verona Heart Study; WHI: Women's Health Initiative. MI: myocardial infarction; CAD: coronary artery disease.

### Genotyping and Quality Control

UK Biobank samples were genotyped using either the UK Bileve (Wain, L. V. et al., Lancet Respir. Med. 3, 769-781 (2015)) or UK Biobank Axiom Arrays having been performed in 33 separate batches of samples by Affymetrix (High Wycombe, UK). A total of 806,466 directly genotyped DNA sequence variants were available after variant quality control (QC). The UK Biobank team then performed imputation from a combined 1000 Genomes/UK10K reference panel; phasing was performed using SHAPEIT-3 and imputation carried out via IMPUTE3. Variant level QC exclusion metrics applied to imputed data for GWAS included: call rate <95%, Hardy-Weinberg Equilibrium P-value <1×10⁻⁶, posterior call probability <0.9, imputation quality <0.4, and minor allele frequency (MAF)<0.005. Sex chromosome and mitochondrial genetic data were excluded from this analysis. In total, 9,061,845 imputed DNA sequence variants were included in our analysis. For sample QC, the UK Biobank analysis team removed individuals of relatedness 3^{rd} degree or higher, and an additional 480 samples with an excess of missing genotype calls or more heterozygosity than expected were excluded. In total, genotypes were available for 120,286 participants of European ancestry.

### Statistical Analysis

### Stage 1 Association Analysis

The BOLT-LMM software (Loh, P. R. et al., Nat Genet 47, 284-90 (2015)) was used to perform linear mixed models (LMMs) for association testing. CAD case status was analyzed while adjusting for age, gender, and chip array at run-time. This analysis was used to derive statistical significance. As effect estimates from BOLT-LMM software are unreliable due to the treatment of binary phenotype data as quantitative data, Applicants performed logistic regression to derive effect estimates for each variant that exceeded genome-wide significance. Effect estimates of top variants were derived from logistic regression using allelic dosages adjusting for age, sex, chip at run-time, and ten principal components under the assumption of additive effects utilizing the R v3.2.0 (www.R-project.org) and SNPTEST (mathgen.stats.ox.ac.uk/genetics_software/snptest/snptest.html) statistical software programs.

### Stage 2 and 3 Meta-Analysis

In stage 2, top variants (P<0.05) from UK Biobank were then meta-analyzed with exome chip data from the CARDIoGRAM Exome Consortium (Myocardial Infarction Genetics and CARDIoGRAM Exome Consortia Investigators. Coding Variation in ANGPTL4, LPL, and SVEP1 and the Risk of Coronary Disease. N Engl J Med 374, 1134-44 (2016)). Tested variants in the CARDIoGRAM exome array study were analyzed through logistic regression with an additive model adjusting for study specific covariates and principal components of ancestry as appropriate. Top variants from UK Biobank that were not available for analysis in the CARDIoGRAM exome array study were then meta-analyzed with data from the 1000 Genomes imputed CARDIoGRAMplusC4D GWAS (CARDIoGRAMplusC4D Consortium. A comprehensive 1000 Genomes-based genome-wide association meta-analysis of coronary artery disease. Nat Genet 47, 1121-30 (2015)) in Stage 3.

Given differences in effect size units between the UK Biobank Stage 1 data and the CARDIoGRAM Exome/1000 Genomes CARDIoGRAMplusC4D data, both Stage 2 and 3 meta-analyses were performed via a weighted z-score method, adjusting for an unbalanced ratio of cases to controls. To derive effect size estimates for variants exceeding genome-wide significance, Applicants meta-analyzed logistic regression results using inverse-variance weighting with fixed effects (METAL software) (Willer et al., Bioinformatics 26, 2190-1 (2010)). Applicants set a combined statistical threshold of P<5×10⁸ for genome wide significance. P values reported in analysis Stages 1, 2, and 3 are all two-sided.

### Phenome-Wide Association Study

For all 15 novel DNA sequence variants associated with CAD in our study, Applicants collaborated with Genomics plc to conduct a phenome-wide association study. This PheWAS used the Genomics plc Platform, UK Biobank, and GTEx Consortium eQTL data. The Genomics plc Platform includes PheWAS data across 545 distinct molecular and disease phenotypes, at an integrated set of over 14 million common variants, from 677 GWAS studies. UK Biobank analyses within the Genomics plc Platform were conducted under a separate research agreement. Applicants selected 25 phenotypes across a range of relevant diseases, metabolic and anthropometric traits from either previously published GWAS datasets or UK Biobank. Complete details of phenotype definitions, sample sizes, and GWAS data sources are shown in Tables 9 and 10. In the PheWAS, quantitative traits were standardized to have unit variance, imputation was performed to generate results for all variants within the 1000 Genomes reference panel, and P values were recalculated based on a Wald test statistic for uniformity.

TABLE 9
Definitions of diseases/traits for PheWAS in 112,338 individuals of European ancestry from
UK Biobank

| | | Sample | |
|---|---|---|---|
| Phenotype | Definition | Size | Covariates |
| Waist Hip | Waist-to-hip ratio measurement at | 112,159 | Age, Body Mass Index, Sex, Principal |
| Ratio Adj | enrollment was quantile-normalized | | Components, Genotyping Chip |
| BMI | separately in males and females, and then | | |
| | combined | | |
| Body Fat | Body fat percentage as measured by an | 110,365 | Age, Body Mass Index, Sex, Principal |
| Percentage | impedance device for body composition | | Components, Genotyping Chip |
| | at enrollment was quantile-normalized | | |
| | separately in males and females, and then | | |
| | combined | | |
| Systolic BP | Automated systolic BP measurement at | 104,611 | Age, Age², Body Mass Index, Sex, |
| | enrollment | | Principal Components, Genotyping Chip |
| Diastolic | Automated diastolic BP measurement at | 104,610 | Age, Age², Body Mass Index, Sex, |
| BP | enrollment | | Principal Components, Genotyping Chip |
| Peripheral | History of peripheral vascular disease or | 692 | Age, Sex, Principal Components, |
| Vascular | intermittent claudication during verbal | | Genotyping Chip |
| Disease | interview or hospitalization for ICD code | | |
| | I731, I738, I739, I743, I744, I745 | | |
| Gout | History of gout during verbal interview | 1612 | Age, Sex, Principal Components, |
| | | | Genotyping Chip |
| Migraine | History of migraine during verbal | 3161 | Age, Sex, Principal Components, |
| | interview | | Genotyping Chip |
| COPD | History of chronic obstructive airway | 2363 | Age, Sex, Principal Components, |
| | disease, emphysema/chronic bronchitis or | | Genotyping Chip |
| | emphysema during verbal interview | | |
| Lung | History of lung cancer, small cell lung | 115 | Age, Sex, Principal Components, |
| Cancer | cancer or non-small cell lung cancer | | Genotyping Chip |
| | during verbal interview | | |
| Breast | History of breast cancer during verbal | 2382 | Age, Sex, Principal Components, |
| Cancer | interview | | Genotyping Chip |
| Colorectal | History of large bowel cancer/colorectal | 616 | Age, Sex, Principal Components, |
| Cancer | cancer, colon cancer/sigmoid cancer or | | Genotyping Chip |
| | rectal cancer during verbal interview | | |
| Any | History of any cancer during verbal | 9530 | Age, Sex, Principal Components, |
| Cancer | interview | | Genotyping Chip |

Abbreviations:
Adj, adjusted;
COPD, chronic obstructive pulmonary disease;
ICD, international classification of disease;
BP, blood pressure

TABLE 10
Characteristics of publicly available GWAS included in phenome-wide association study.

| | Outcome/Trait | | |
|---|---|---|---|
| Consortium | (Units) | Sample Size | Genotyping |
| GLGC (Global Lipids Genetics | LDL cholesterol (SD) | Up to 188,587 | 37 studies |
| Consortium et al. Discovery and | HDL cholesterol | individuals | using |
| refinement of loci associated with | (SD) | | metabochip, 23 |
| lipid levels. Nat Genet 45, 1274-83 | Total cholesterol | | studies using |
| (2013)) | (SD) | | various arrays |
| | Triglycerides (SD) | | |
| MAGIC (Manning, A.K. et al. A | Fasting Insulin | Up to 96,496 | Various arrays, |
| genome-wide approach accounting | Adjusted for BMI | individuals | imputation to |
| for body mass index identifies | (SD) | | 2.5 million |
| genetic variants influencing fasting | | | SNPs using |
| glycemic traits and insulin | | | HapMap |
| resistance. Nat Genet 44, 659-69 | | | reference panel |
| (2012)) | | | |
| MAGIC (Prokopenko, I. et al. A | Insulin Secretion | Up to 5,318 | Various Arrays |
| central role for GRB10 in | (SD) | individuals | imputation to |
| regulation of islet function in man. | | | 2.4 million |
| PLoS Genet 10, e1004235 (2014)) | | | SNPs using |
| | | | HapMap |
| | | | reference panel |
| GIANT (Wood, A.R. et al. | Height (SD) | Up to 253,288 | Various arrays, |
| Defining the role of common | | individuals | imputation to |
| variation in the genomic and | | | 2.5 million |
| biological architecture of adult | | | SNPs using |
| human height. Nat Genet 46, 1173- | | | HapMap |
| 86 (2014)) | | | reference panel |
| GIANT(Berndt, S.I. et al. Genome- | Body Mass Index | Up to 263,407 | Various arrays, |
| wide meta-analysis identifies 11 | (OR) | individuals total, | imputation to |
| new loci for anthropometric traits | | focusing on the | 2.8 million |
| and provides insights into genetic | | upper 5^{th} | SNPs |
| architecture. Nat Genet 45, 501-12 | | percentile (cases) | |
| (2013)) | | and lower 5th | |
| | | percentile | |
| | | (controls) of BMI | |
| | | the distribution | |
| CKDGen (Pattaro, C. et al. Genetic | Cystatin C/Creatinine | Up to 133,413 | Various arrays, |
| associations at 53 loci highlight | Serum estimated | individuals | imputation to |
| cell types and biological pathways | Glomerular Filtration | | 2.5 million |
| relevant for kidney function. Nat | Rate | | SNPs using |
| Commun 7, 10023 (2016)) | (mL/min/1.73 m2) | | HapMap |
| | | | reference panel |
| IIBDGC (Liu, J.Z. et al. | Inflammatory Bowel | Up to 38,155 | Various arrays, |
| Association analyses identify 38 | Disease (OR) | cases and 48,485 | imputation to 9 |
| susceptibility loci for | | controls of | million SNPs |
| inflammatory bowel disease and | | European | using 1000 |
| highlight shared genetic risk across | | Ancestry | Genomes |
| populations. Nat Genet 47, 979-86 | | | reference panel |
| (2015)) | | | |
| ADIPOGen (Dastani, Z. et al. | Adiponectin (SD) | Up to 39,883 | Various arrays, |
| Novel loci for adiponectin levels | | individuals of | imputation to |
| and their influence on type 2 | | European | 2.7 million |
| diabetes and metabolic traits: a | | Ancestry | SNPs using |
| multi-ethnic meta-analysis of | | | HapMap |
| 45,891 individuals. PLoS Genet 8, | | | reference panel |
| e1002607 (2012)) | | | |
| DIAGRAM (Morris, A.P. et al. | Type 2 Diabetes | Meta-analysis of | Various arrays, |
| Large-scale association analysis | (OR) | up to 34,840 cases | imputation to |
| provides insights into the genetic | | and 114,981 | 2.5 million |
| architecture and pathophysiology | | controls in | SNPs using |
| of type 2 diabetes. Nat Genet 44, | | individuals of | HapMap |
| 981-90 (2012)) | | primarily | reference panel |
| | | European | |
| | | Ancestry | |

DIAGRAM, DIAbetes Genetics Replication And Meta-analysis; GIANT, Genetic Investigation of ANthropometric Traits; GLGC, Global Lipids Genetics Consortium; MAGIC, Meta-Analyses of Glucose and Insulin-related traits Consortium (data on glycemic traits have been contributed by MAGIC investigators and have been downloaded from www.magicinvestigators.org); CKDGen, Chronic Kidney Disease Genetics Consortium; IMDGC, International Inflammatory Bowel Disease Genetics Consortium; SNPs, single nucleotide polymorphism; LDL cholesterol, low-density lipoprotein cholesterol; HDL cholesterol, high-density lipoprotein cholesterol; SD, standard deviation; BMI, body mass index; OR, odds ratio.

Phenotypes were declared to be significantly associated with the risk variant if they met a Bonferroni corrected P value of <0.00013 [0.05/(25 traits x 15 DNA sequence variants)]. Phenome scan results were then depicted in a heatmap based on the Z-scores for all variant-disease/trait associations aligned to the CAD risk allele as implemented by the gplots package (cran.r-project.org/web/packages/gplots/gplots.pdf) in R v3.2.0 To identify loci that might influence gene expression, Applicants used previously published cis-expression quantitative trait locus (eQTL) mapping data from the Genotype-Tissue Expression (GTEx) Consortium Project across 44 tissues. Applicants queried the 15 novel variants identified in our study for overlap with genome-wide significant variant-gene pairs from the GTEx portal (gtexportal.org).

### Allele Specific Expression Analysis

Allele-specific expression (ASE) data from the GTEx project were obtained from dbGaP (accession phs000424.v6.p1). The generation of these data is summarized in Aguet et al., and relied on methods described earlier. In brief, only uniquely mapping reads with base quality ≥10 at the SNP were counted, and only SNPs with coverage of at least 8 reads were reported. For ARHGEF26 p.Val29Leu, ASE counts were available for 20 heterozygous individuals. A two-sided binomial test was used to identify SNPs with significant allelic imbalance in each individual, and Benjamini-Hochberg adjusted p-values were calculated across all sites measured in an individual.

### Luciferase Reporter Assay

HUVEC heterozygous for rs12493885 were identified from Caucasian donors by SNP genotyping. A 2.9 kb genomic fragment spanning from 5′ upstream of ARHGEF26 to exon 2 (rs12493885) was cloned into a pMiniT 2.0 vector (NEB) using the heterozygous HUVEC genomic DNA as a template, and sequenced for reference and alternative alleles. The −2516 to +2 reference and alternative haplotypes upstream of ARHGEF26 (NC_000003.12:154119477-154121994) were amplified from the 2.9 kb region by PCR with primers designed to create 5′ NheI and 3′ HindIII restriction sites in the PCR products. The amplified fragments were subcloned between the NheI and HindIII sites of a promoterless firefly luciferase (luc2) expression vector pGL4.10 (Promega), to create two plasmids: pGL4.10-Ref and pGL4.10-Alt. Promoterless pGL4.10-control, and pGL4.73[hRluc/SV40] vector containing the Renilla luciferase hRluc reporter gene and an SV40 early enhancer/promoter, were used as negative control and co-reporter, respectively. Cells were cotransfected with equal amounts of luc2 expression plasmid (pGL4.10-control, pGL4.10-Ref and pGL4.10-Alt) and pGL4.73 vector by Lipofectamine 2000. Cells were harvested at 48 h after transfection and followed by a Dual-Glo Luciferase Assay (Promega) to measure firefly and Renilla luciferase activities. The firefly luciferase activity was normalized to Renilla luciferase in the same sample, and expressed as fold change relative to pGL4.10-control group.

### Nucleotide Exchange Assay

Human full-length ARHGEF26 (wild-type or 29Leu) and RhoG (residues 1-188) proteins, both with N-terminal His-SUMO tags, were expressed in E. coli BL21(DE3) cells in TB media. Nucleotide exchange assay samples were prepared in buffer containing 10 mM HEPES pH 7.4, 150 mM NaCl, 1 mM MgCl₂, 0.5 uM MANT-GTP, 2 mM TCEP with 1 μM ARHGEF26. Just prior to reading, RhoG protein, pre-loaded with GDP, was added to a final concentration of 0.4 M. MANT-GTP fluorescence was monitored for 60 minutes on a SpectraMax M2 at 37° C. using an excitation wavelength of 280 nm and an emissions wavelength of 440 nm with a 435 nm cutoff. Fluorescence data was imported into Prism GraphPad for analysis.

Functional Characterization of ARHGEF26 p. Val29Leu in Arterial Tissue

To investigate the functional effects of ARHGEF26 p.Val29Leu (rs12493885), Applicants knocked-down the expression of endogenous ARHGEF26 in cultured human aortic endothelial cells (HAEC) and human coronary artery smooth muscle cells (HCASMC) by RNA interference. Applicants then overexpressed wild-type or mutant ARHGEF26 (29Leu) resistant to siRNA, and measured leukocyte transendothelial migration, leukocyte adhesion on endothelial cells, and HCASMC proliferation in vitro. Applicants also evaluated the degradation of wild-type or 29Leu mutant ARHGEF26 with a cycloheximide chase assay and Western blotting.

Cell Culture

Human Aortic Endothelial Cells (HAEC), Human Umbilical Vein Endothelial Cells (HUVEC), and Human Coronary Artery Smooth Muscle Cells (HCASMC) were purchased from Lifeline Cell Technology and maintained in VascuLife EnGS Endothelial Medium and SMC Medium (Lifeline Cell Technology) free of antibiotics at 37° C. and 5% CO₂. HAEC, HUVEC, and HCASMC at passages 2-6 were used for experiments. HL60 cell line was purchased from Sigma-Aldrich. HEK293 and THP-1 cell lines were purchased from ATCC. HEK293 was maintained in high-glucose Dulbecco's Modified Eagle Medium with GluaMA Supplement and 10% fetal bovine serum (Thermo Fisher Scientific). HL.60 and THP-1 cells were maintained in RPMI 1640 Medium supplemented with 10% non-heated-inactivated fetal bovine serum (Thermo Fisher Scientific). HL60 cells were differentiated for 5 days in medium containing 1.3% DMSO for leukocyte TEM assays. Cell line specificity was confirmed with tissue-specific markers: HAEC were von Willebrand Factor positive and smooth muscle a-actin negative, HCASMC were von Willebrand Factor negative and smooth muscle a-actin positive. Both cell types were confirmed to be mycoplasma negative.

siRNA and ARHGEF26 Constructs

Silencer Select siRNA against 3′UTR of human ARHGEF26 was customized from Thermo Fisher Scientific. Targeting efficiency of siRNA was confirmed by western blot of transfected cells. Non-targeting siRNA control was purchased from Thermo Fisher Scientific. The cDNA containing the complete open-reading frame of human ARHGEF26 (NM_015595.3) was obtained from the Mammalian Gene Collection (MGC) and cloned with an N-terminal FLAG-GGGS sequence onto a pcDNA3.4 mammalian expression vector (Thermo Fisher Scientific) using NEBuilder HiFi DNA Assembly Master Mix (NEB). Wild-type ARHGEF26 and 29Leu mutant was generated by site-directed mutagenesis (Q5 kit, NEB) and sanger-sequenced. Vector without FLAG-GGGS-ARHGEF26 insert is used as control vector.

Transfection

HAEC and HCASMC were transfected in 6-well format using Lipofectamine 2000 Transfection Reagent (Invitrogen) following manufacture's protocol. Briefly, cells were plated at 90% confluency the day prior to transfection. Then cells were washed and replenished with Opti-MEM I Reduced Serum Medium. Per well, cells were co-transfected with 50 nM siRNA with 1 ug/mL ARHGEF26 vector (final concentration). Medium was replaced at 4 hours post-transfection. Cells were trypsinized and re-plated one-day after transfection (HAEC), or re-plated and starved in serum-free medium (HCASMC).

Leukocyte TEM Assay

Leukocyte TEM assay was modified from previously described (van Buul, J. D. et al., J Cell Biol 178, 1279-93 (2007)). HAEC was plated on a HTS Transwell 96-well permeable insert with 5.0 μm pore size (Corning) in 40 μL/well medium and allowed to settle for 8 hours. Then the transwell was replaced with complete medium contain 10 ng/mL TNF-α (PeproTech) and cultured overnight. The next day, 235 μL/well serum-free endothelial cell medium containing 0.25% BSA with vehicle or 50 ng/mL SDF-1 (PeproTech) was placed on a 96-well white receiver plate. The medium in the transwell insert was removed and replaced with 75 μL/well serum-free endothelial cell medium containing 0.25% BSA and 200,000 differentiated HL60 cells. The insert was then gently placed in the receiver plate and incubated at 37° C. for 5 hours with lid on. The insert was removed and HL60 migrated into the receiver plate was quantified with a luminescent assay (CellTiter-Glo, Promega). Standard curve of HL60 cells was prepared by serial dilutions on an identical white receiver plate, with total HL60 cell input set as 100%. Differences in means of percentage of migrated cells per well were assessed by two-way ANOVA with uncorrected Fisher's LSD test within vehicle and SDF-1 subgroups, respectively, and significance threshold set as P<0.05.

Leukocyte Adhesion Assay

HAEC were transfected and re-plated on a black-wall, clear-bottom 96-well plate and cultured until 100% confluence (48-72-hour post-transfection). Prior to the assay, HAEC were treated with 10 ng/mL TNF-α overnight. THP-1 cells were labeled with Calcein-AM cell-permeant dye (Thermo Fisher Scientific), washed, and added to wells containing HAEC at 200,000/well in serum-free medium containing 0.25% BSA, and incubated at 37° C. for 1 hour. The wells were washed four times in 37° C. PBS. After the final wash, the plate was drained thoroughly and 100 μL TBS buffer containing 1% NP-40 was added to each well. The plate was agitated for 10 min protected from light, and the fluorescence was measured on a plate reader. Standard curve was generated on an identical, separate plate. Differences in means of fluorescent intensity were assessed by one-way ANOVA with Dunnett's multiple comparisons test, and a multiplicity adjusted P value set as 0.05 for statistical significance.

VSMC Proliferation

HCASMC were transfected and re-plated on a 96-well plate in serum-free medium and starved. After 48 hours, the plate was replaced with medium containing serum and cells are allowed to proliferate for 72 hours. To measure cell proliferation, the medium was removed and cell numbers in each well were counted with a luminescent assay (CellTiter-Glo, Promega). Differences in means of luminescence were assessed by one-way ANOVA with Dunnett's multiple comparisons test, and a multiplicity adjusted P value set as 0.05 for statistical significance.

Western Blot

Cells were harvested with lysis buffer (150 mM NaCl, 50 mM Tris HCl, 0.5% NP-40 and 0.1% sodium deoxycholate, pH 7.5) supplemented with fresh protease inhibitors (Pierce Protease Inhibitor Mini Tablet, EDTA free). Cell lysate was incubated for 15 min in rotation and centrifuged at 20,000 g for 15 min at 4° C. to remove insoluble materials. The protein concentration in the supernatant was measured by a bicinchoninic acid (BCA) assay kit (Thermo Fisher Scientific) and normalized with Laemmli sample buffer. Equal amount of protein was separated by sodium dodecyl sulfate polyacrylamide gel electrophoresis (SDS-PAGE) on 4-20% Mini-PROTEAN TGX precast gels (Bio-Rad Laboratories), transferred to nitrocellulose membrane, and blocked with 5% non-fat milk in Tris-buffered saline supplemented with 0.05% Tween-20 (TBST) at room temperature for 1 hour. The membrane was then probed with primary antibodies to ARHGEF26 (Sigma-Aldrich), FLAG (M2 HRP-conjugated, Sigma-Aldrich), or actin (HRP-conjugated, Santa Cruz Biotechnology), respectively, in 1% non-fat milk in TBST. The HRP-conjugated anti-rabbit secondary antibody was then incubated at room temperature for 1 hour for ARHGEF26 blots. After extensive washing, the membranes were imaged by an enhanced chemiluminescence substrate (EMD Millipore) and imaged on Amersham Imager 600 (GE Healthcare).

Cycloheximide Chase Assay

FLAG-tagged WT or 29Leu FLAG-ARHGEF26 was overexpressed in HEK293 cells for 48 hours. One day prior to the cycloheximide chase, WT and 29Leu ARHGEF26-transfected cells (12 wells each) were plated on the same 24-well plate at 150,000 cells per well in 500 μL medium. For the cycloheximide chase, 500 μL medium containing 100 μg/mL or 200 μg/mL cycloheximide (Enzo Life Sciences) was added to each well to achieve 50 μg/mL or 100 μg/mL final concentration. Cells were harvested in lysis buffer at indicated time points post chase, and BCA-normalized lysate (20 μg/time points) were probed for FLAG by Western blot. For each cycloheximide dose, 2 blot sections (WT and 29Leu) from the same treated plate were blotted on same membrane and simultaneously imaged.

Data Availability

Stage 2 and Stage 3 data contributed by CARDIoGRAM Exome and CARDIoGRAMplusC4D investigators is available at www.CARDIOGRAMPLUSC4D.ORG. The genetic and phenotypic UK Biobank data are available upon application to the UK Biobank (www.ukbiobank.ac.uk/).

TABLE 11
variants linked to risk of myocardial infarction at ‘genome-wide’
level of statistical significance from a literature-based survey.

| | | | pos | | | | | polygenic |
|---|---|---|---|---|---|---|---|---|
| | representative | | basepair | risk | nonrisk | risk allele | odds | score |
| locus | variant rsid | chromosome | b37 | allele | allele | frequency | ratio | weight |
| COL4A1- | rs4773144 | 13 | 110960712 | G | A | 0.44 | 1.07 | 0.029383778 |
| COL4A2 | | | | | | | | |
| MIA3 | rs17465637 | 1 | 222823529 | C | A | 0.51 | 1.2 | 0.079181246 |
| REST-NOA1 | rs17087335 | 4 | 57838583 | T | G | 0.21 | 1.06 | 0.025305865 |
| ZC3HC1 | rs11556924 | 7 | 129663496 | C | T | 0.62 | 1.09 | 0.037426498 |
| CDKN2A- | rs1333049 | 9 | 22125503 | C | G | 0.42 | 1.27 | 0.103803721 |
| CDKN2B | | | | | | | | |
| PDGFD | rs974819 | 11 | 103660567 | A | G | 0.29 | 1.07 | 0.029383778 |
| SWAP70 | rs10840293 | 11 | 9751196 | A | G | 0.55 | 1.06 | 0.025305865 |
| KSR2 | rs11830157 | 12 | 118265441 | G | T | 0.36 | 1.12 | 0.049218023 |
| ADAMTS7 | rs3825807 | 15 | 79089111 | A | G | 0.57 | 1.08 | 0.033423755 |
| BCAS3 | rs7212798 | 17 | 59013488 | C | T | 0.15 | 1.08 | 0.033423755 |
| FLT1 | rs9319428 | 13 | 28973621 | A | G | 0.32 | 1.05 | 0.021189299 |
| IL6R | rs4845625 | 1 | 154422067 | T | C | 0.47 | 1.04 | 0.017033339 |
| CXCL12 | rs501120 | 10 | 44753867 | T | C | 0.67 | 1.33 | 0.123851641 |
| SH2B3 | rs3184504 | 12 | 50792403 | T | C | 0.4 | 1.07 | 0.029383778 |
| SMAD3 | rs17228212 | 15 | 67458639 | C | T | 0.13 | 1.21 | 0.08278537 |
| SORT1 | rs599839 | 1 | 109822166 | A | G | 0.64 | 1.29 | 0.11058971 |
| PCSK9 | rs11206510 | 1 | 55496039 | T | C | 0.81 | 1.15 | 0.06069784 |
| APOB | rs515135 | 2 | 21286057 | G | A | 0.83 | 1.08 | 0.033423755 |
| ABCG5- | rs6544713 | 2 | 44073881 | T | C | 0.3 | 1.06 | 0.025305865 |
| ABCG8 | | | | | | | | |
| LIPA | rs2246833 | 10 | 91005854 | T | C | 0.38 | 1.06 | 0.025305865 |
| LDLR | rs1122608 | 19 | 11163601 | G | T | 0.75 | 1.15 | 0.06069784 |
| APOE-APOC1 | rs2075650 | 19 | 45395619 | G | A | 0.14 | 1.11 | 0.045322979 |
| SLC22A3- | rs3798220 | 6 | 160961137 | C | T | 0.02 | 1.51 | 0.178976947 |
| LPAL2-LPA | | | | | | | | |
| LPL | rs264 | 8 | 19813180 | G | A | 0.86 | 1.05 | 0.021189299 |
| TRIB1 | rs2954029 | 8 | 126490972 | A | T | 0.55 | 1.04 | 0.017033339 |
| ZNF259- | rs964184 | 11 | 116648917 | G | C | 0.13 | 1.13 | 0.053078443 |
| APOA5/A4/C3/A1 | | | | | | | | |
| ANGPTL4 | rs116843064 | 12 | 8429323 | G | A | 0.98 | 1.16 | 0.064457989 |
| PPAP2B | rs17114036 | 1 | 56962821 | A | G | 0.91 | 1.17 | 0.068185862 |
| WDR12 | rs6725887 | 2 | 203745885 | C | T | 0.14 | 1.17 | 0.068185862 |
| VAMP5- | rs1561198 | 2 | 85809989 | A | G | 0.45 | 1.05 | 0.021189299 |
| VAMP8- | | | | | | | | |
| GGCX | | | | | | | | |
| ZEB2- | rs2252641 | 2 | 145801461 | G | A | 0.46 | 1.04 | 0.017033339 |
| AC074093.1 | | | | | | | | |
| AK097927 | rs16986953 | 2 | 19942473 | A | G | 0.19 | 1.17 | 0.068185862 |
| MRAS | rs2306374 | 3 | 138119952 | C | T | 0.18 | 1.12 | 0.049218023 |
| SLC22A4- | rs273909 | 5 | 131667353 | C | T | 0.14 | 1.09 | 0.037426498 |
| SLC22A5 | | | | | | | | |
| ANKS1A | rs17609940 | 6 | 35034800 | G | C | 0.75 | 1.07 | 0.029383778 |
| PHACTR1 | rs12526453 | 6 | 12927544 | C | G | 0.65 | 1.12 | 0.049218023 |
| TCF21 | rs12190287 | 6 | 134214525 | C | G | 0.62 | 1.08 | 0.033423755 |
| KCNK5 | rs10947789 | 6 | 39174922 | T | C | 0.76 | 1.06 | 0.025305865 |
| PLG | rs4252120 | 6 | 161143608 | T | C | 0.73 | 1.06 | 0.025305865 |
| HDAC9 | rs2023938 | 7 | 19036775 | G | A | 0.1 | 1.07 | 0.029383778 |
| ABO | rs579459 | 9 | 136154168 | C | T | 0.21 | 1.1 | 0.041392685 |
| SVEP1 | rs111245230 | 9 | 113169775 | C | T | 0.036 | 1.14 | 0.056904851 |
| CYP17A1- | rs12413409 | 10 | 104719096 | G | A | 0.89 | 1.12 | 0.049218023 |
| CNNM2- | | | | | | | | |
| NT5C2 | | | | | | | | |
| KIAA1462 | rs2505083 | 10 | 30335122 | C | T | 0.42 | 1.06 | 0.025305865 |
| ATP2B1 | rs7136259 | 12 | 90081188 | T | C | 0.43 | 1.08 | 0.033423755 |
| HHIPL1 | rs2895811 | 14 | 100133942 | C | T | 0.43 | 1.07 | 0.029383778 |
| MFGE8- | rs8042271 | 15 | 89574218 | G | A | 0.9 | 1.1 | 0.041392685 |
| ABHD2 | | | | | | | | |
| SMG6-SRR | rs216172 | 17 | 2126504 | C | G | 0.37 | 1.07 | 0.029383778 |
| RASD1- | rs12936587 | 17 | 17543722 | G | A | 0.56 | 1.07 | 0.029383778 |
| SMCR3- | | | | | | | | |
| PEMT | | | | | | | | |
| UBE2Z-GIP- | rs46522 | 17 | 46988597 | T | C | 0.53 | 1.06 | 0.025305865 |
| ATP5G1- | | | | | | | | |
| SNF8 | | | | | | | | |
| PMAIP1- | rs663129 | 18 | 57838401 | A | G | 0.26 | 1.06 | 0.025305865 |
| MC4R | | | | | | | | |
| ZNF507- | rs12976411 | 19 | 32882020 | A | T | 0.91 | 1.49 | 0.173186268 |
| LOC400684 | | | | | | | | |
| SLC5A3- | rs9982601 | 21 | 35599128 | T | C | 0.13 | 1.2 | 0.079181246 |
| MRPS6- | | | | | | | | |
| KCNE2 | | | | | | | | |
| POM121L9P- | rs180803 | 22 | 24658858 | G | T | 0.97 | 1.2 | 0.079181246 |
| ADORA2A | | | | | | | | |
| GUCY1A3 | rs7692387 | 4 | 156635309 | G | A | 0.81 | 1.06 | 0.025305865 |
| EDNRA | rs1878406 | 4 | 148393664 | T | C | 0.15 | 1.06 | 0.025305865 |
| NOS3 | rs3918226 | 7 | 150690176 | T | C | 0.06 | 1.14 | 0.056904851 |
| FURIN-FES | rs17514846 | 15 | 91416550 | A | C | 0.44 | 1.05 | 0.021189299 |
| (LOC646736) | rs2972146 | 2 | 227100698 | T | G | 0.65 | 1.06 | 0.025305865 |
| ARHGEF26 | rs12493885 | 3 | 153839866 | C | G | 0.85 | 1.08 | 0.033423755 |
| LOX | rs1800449 | 5 | 121413208 | T | C | 0.17 | 1.07 | 0.029383778 |
| CCDC92 | rs11057401 | 12 | 124427306 | T | A | 0.69 | 1.06 | 0.025305865 |
| FN1 | rs17517928 | 2 | 216291359 | C | T | 0.75 | 1.06 | 0.025305865 |
| UMPS-ITGB5 | rs17843797 | 3 | 124453022 | G | T | 0.13 | 1.07 | 0.029383778 |
| FGD5 | rs748431 | 3 | 14928077 | G | T | 0.36 | 1.05 | 0.021189299 |
| RHOA | rs7623687 | 3 | 49448566 | A | C | 0.86 | 1.08 | 0.033423755 |
| (FGF5) | rs10857147 | 4 | 81181072 | T | A | 0.29 | 1.06 | 0.025305865 |
| (MAD2L1) | rs7678555 | 4 | 120909501 | C | A | 0.29 | 1.06 | 0.025305865 |
| RP11- | rs10841443 | 12 | 20220033 | G | C | 0.67 | 1.05 | 0.021189299 |
| 664H17.1 | | | | | | | | |
| HNF1A | rs2244608 | 12 | 121416988 | G | A | 0.32 | 1.05 | 0.021189299 |
| CFDP1 | rs3851738 | 16 | 75387533 | C | G | 0.6 | 1.05 | 0.021189299 |
| CDH13 | rs7500448 | 16 | 83045790 | A | G | 0.75 | 1.06 | 0.025305865 |
| TGFB1 | rs8108632 | 19 | 41854534 | T | A | 0.41 | 1.05 | 0.021189299 |
| KCNJ13- | rs1801251 | 2 | 233633460 | A | G | 0.35 | 1.05 | 0.021189299 |
| GIGYF2 | | | | | | | | |
| C2 | rs3130683 | 6 | 31888367 | T | C | 0.86 | 1.09 | 0.037426498 |
| MRVI1-CTR9 | rs11042937 | 11 | 10745394 | T | G | 0.49 | 1.04 | 0.017033339 |
| LRP1 | rs11172113 | 12 | 57527283 | C | T | 0.41 | 1.06 | 0.025305865 |
| SCARB1 | rs11057830 | 12 | 125307053 | A | G | 0.15 | 1.08 | 0.033423755 |
| CETP | rs1800775 | 16 | 56995236 | C | A | 0.51 | 1.05 | 0.021189299 |
| ATP1B1 | rs1892094 | 1 | 169094459 | C | T | 0.5 | 1.04 | 0.017033339 |
| DDX59- | rs6700559 | 1 | 200646073 | C | T | 0.53 | 1.04 | 0.017033339 |
| CAMSAP2 | | | | | | | | |
| LMOD1 | rs2820315 | 1 | 201872264 | T | C | 0.3 | 1.05 | 0.021189299 |
| TNS1 | rs2571445G | 2 | 218683154 | A | G | 0.39 | 1.05 | 0.021189299 |
| ARHGAP26 | rs246600 | 5 | 142516897 | T | C | 0.48 | 1.04 | 0.017033339 |
| PARP12 | rs10237377 | 7 | 139757136 | G | T | 0.65 | 1.05 | 0.021189299 |
| PCNX3 | rs12801636 | 11 | 65391317 | G | A | 0.77 | 1.05 | 0.021189299 |
| SERPINH1 | rs590121 | 11 | 75274150 | T | G | 0.65 | 1.05 | 0.021189299 |
| C12orf43- | rs2258287 | 12 | 121454313 | A | C | 0.34 | 1.04 | 0.017033339 |
| HNF1A | | | | | | | | |
| SCARB1 | rs11057830 | 12 | 125307053 | A | G | 0.16 | 1.06 | 0.025305865 |
| OAZ2, RBPMS2 | rs6494488 | 15 | 65024204 | A | G | 0.82 | 1.05 | 0.021189299 |
| DHX38 | rs1050362 | 16 | 72130815 | A | C | 0.38 | 1.04 | 0.017033339 |
| GOSR2 | rs17608766 | 17 | 45013271 | C | T | 0.14 | 1.07 | 0.029383778 |
| PECAM1 | rs1867624 | 17 | 62387091 | T | C | 0.61 | 1.04 | 0.017033339 |
| PROCR | rs867186 | 20 | 33764554 | A | G | 0.89 | 1.08 | 0.033423755 |

### Example 2—Genetic Risk, Adherence to a Healthy Lifestyle, and Coronary Disease

Both genetic and lifestyle factors are key drivers of coronary artery disease, a complex disorder that is the leading cause of death worldwide. (Lozano R, Naghavi M, Foreman K, et al. Global and regional mortality from 235 causes of death for 20 age groups in 1990 and 2010: a systematic analysis for the Global Burden of Disease Study 2010. Lancet 2012; 380:2095-2128) A familial pattern in the risk of coronary artery disease was first described in 1938 and was subsequently confirmed in large studies involving twins and prospective cohorts. www.nejm.org/doi/full/10.1056/NEJMoa1605086—ref2 (Müller C. Xanthomata, hypercholesterolemia, angina pectoris. Acta Med Scand 1938; 89:75-84; Gertler M M, Garn S M, White P D. Young candidates for coronary heart disease. J Am Med Assoc 1951; 147:621-625; Slack J, Evans K A. The increased risk of death from ischaemic heart disease in first degree relatives of 121 men and 96 women with ischaemic heart disease. J Med Genet 1966; 3:239-257; Marenberg M E, Risch N, Berkman L F, Floderus B, de Faire U. Genetic susceptibility to death from coronary heart disease in a study of twins. N Engl J Med 1994; 330:1041-1046; Lloyd-Jones D M, Nam B H, D'Agostino R B Sr, et al. Parental cardiovascular disease as a risk factor for cardiovascular disease in middle-aged adults: a prospective study of parents and offspring. JAMA 2004; 291:2204-2211). Since 2007, genomewide association analyses have identified more than 50 independent loci associated with the risk of coronary artery disease. (Sarnani N J, Erdmann J, Hall A S, et al. Genomewide association analysis of coronary artery disease. N Engl J Med 2007; 357:443-453; Helgadottir A, Thorleifsson G, Manolescu A, et al. A common variant on chromosome 9p21 affects the risk of myocardial infarction. Science 2007; 316:1491-1493; McPherson R, Pertsemlidis A, Kavaslar N, et al. A common allele on chromosome 9 associated with coronary heart disease. Science 2007; 316:1488-1491; Myocardial Infarction Genetics Consortium. Genome-wide association of early-onset myocardial infarction with single nucleotide polymorphisms and copy number variants. Nat Genet 2009; 41:334-341; Erdmann J, Grosshennig A, Braund P S, et al. New susceptibility locus for coronary artery disease on chromosome 3q22.3. Nat Genet 2009; 41:280-282; Coronary Artery Disease (C4D) Genetics Consortium. A genome-wide association study in Europeans and South Asians identifies five new loci for coronary artery disease. Nat Genet 2011; 43:339-344; IBC 50K CAD Consortium. Large-scale gene-centric analysis identifies novel variants for coronary artery disease. PLoS Genet 2011; 7:e1002260-e1002260; The CARDIoGRAMplusC4D Consortium. Large-scale association analysis identifies new risk loci for coronary artery disease. Nat Genet 2013; 45:25-33; Nikpay M, Goel A, Won H H, et al. A comprehensive 1,000 Genomes-based genome-wide association meta-analysis of coronary artery disease. Nat Genet 2015; 47:1121-1130). These risk alleles, when aggregated into a polygenic risk score, are predictive of incident coronary events and provide a continuous and quantitative measure of genetic susceptibility. (Kathiresan S, Melander O, Anevski D, et al. Polymorphisms associated with cholesterol and risk of cardiovascular events. N Engl J Med 2008; 358:1240-1249; Ripatti S, Tikkanen E, Orho-Melander M, et al. A multilocus genetic risk score for coronary heart disease: case-control and prospective cohort analyses. Lancet 2010; 376:1393-1400; Paynter N P, Chasman D I, Pare G, et al. Association between a literature-based genetic risk score and cardiovascular events in women. JAMA 2010; 303:631-637; Thanassoulis G, Peloso G M, Pencina M J, et al. A genetic risk score is associated with incident cardiovascular disease and coronary artery calcium: the Framingham Heart Study. Cire Cardiovasc Genet 2012; 5:113-121; Brautbar A, Pompeii L A, Dehghan A, et al. A genetic risk score based on direct associations with coronary heart disease improves coronary heart disease risk prediction in the Atherosclerosis Risk in Communities (ARIC), but not in the Rotterdam and Framingham Offspring, Studies. Atherosclerosis 2012; 223:421-426; Ganna A, Magnusson P K, Pedersen N L, et al. Multilocus genetic risk scores for coronary heart disease prediction. Arterioscler Thromb Vasc Biol 2013; 33:2267-2272; Mega J L, Stitziel N O, Smith J G, et al. Genetic risk, coronary heart disease events, and the clinical benefit of statin therapy: an analysis of primary and secondary prevention trials. Lancet 2015; 385:2264-2271; Tada H, Melander O, Louie J Z, et al. Risk prediction by genetic risk scores for coronary heart disease is independent of self-reported family history. Eur Heart J 2016; 37:561-567; Abraham G, Havulinna A S, Bhalala O G, et al. Genomic prediction of coronary heart disease. Eur Heart J. 2016 Nov. 14; 37(43):3267-3278).

Much evidence has also shown that persons who adhere to a healthy lifestyle have markedly reduced rates of incident cardiovascular events. (Starnpfer M J, Hu F B, Manson J F, Rimm E B, Willett W C. Primary prevention of coronary heart disease in women through diet and lifestyle. N Engl J Med 2000; 343:16-22; Folsom A R, Yatsuya H, Nettleton J A, Lutsey P L, Cushman M, Rosamond W D. Community prevalence of ideal cardiovascular health, by the American Heart Association definition, and relationship with cardiovascular disease incidence. J Am Coll Cardiol 2011; 57:1690-1696; Yang Q, Cogswell M E, Flanders W D, et al. Trends in cardiovascular health metrics and associations with all-cause and CVD mortality among US adults. JAMA 2012; 307:1273-1283; Xanthakis V, Elnserro D M, Murabito J M, et al. Ideal cardiovascular health: associations with biomarkers and subclinical disease and impact on incidence of cardiovascular disease in the Framingham Offspring Study. Circulation 2014; 130:1676-1683; Chomistek A K, Chiuve S E, Eliassen A H, Mukamal K J, Willett W C, Rimm E B. Healthy lifestyle in the primordial prevention of cardiovascular disease among young women. J Am Coll Cardiol 2015; 65:43-51; Akesson A, Larsson S C, Discacciati A, Wolk A. Low-risk diet and lifestyle habits in the primary prevention of myocardial infarction in men: a population-based prospective cohort study. 3 Am (Coll Cardiol 2014; 64:1299-1306). The promotion of healthy lifestyle behaviors, which include not smoking, avoiding obesity, regular physical activity, and a healthy diet pattern, underlies the current strategy to improve cardiovascular health in the general population. www.nejm.org/doi/full/10.1056/NEJMoa1605086—ref31 (Lloyd-Jones D M, Hong Y, Labarthe D, et al. Defining and setting national goals for cardiovascular health promotion and disease reduction: the American Heart Association's strategic Impact Goal through 2020 and beyond. Circulation 2010; 121:586-613).

Many observers assume that a genetic predisposition to coronary artery disease is deterministic. (White P D. Genes, the heart and destiny. N Engl J Med 1957; 256:965-969). However, genetic risk might be attenuated by a favorable lifestyle. Here, Applicant analyzed data for participants in three prospective cohorts and one cross-sectional study to test the hypothesis that both genetic factors and baseline adherence to a healthy lifestyle contribute independently to the risk of incident coronary events and the prevalent subclinical burden of atherosclerosis. Applicant then determined the extent to which a healthy lifestyle is associated with a reduced risk of coronary artery disease among participants with a high genetic risk.

### Methods

### Study Populations

The Atherosclerosis Risk in Communities (ARIC) study is a prospective cohort that enrolled white participants and black participants between the ages of 45 and 64 years, starting in 1987. (The Atherosclerosis Risk in Communities (ARIC) Study: design and objectives. Am J Epidemiol 1989; 129:687-702). For data from this study, Applicant retrieved genotype and clinical data from the National Center for Biotechnology Information dbGAP server (accession number, phs000280.v3.p1). The Women's Genome Health Study (WGHS) is a prospective cohort of female health professionals derived from the Women's Health Study, a clinical trial initiated in 1992 to evaluate the efficacy of aspirin and vitamin E in the primary prevention of cardiovascular disease. (Ridker P M, Chasman D I, Zee R Y, et al. Rationale, design, and methodology of the Women's Genome Health Study: a genome-wide association study of more than 25,000 initially healthy American women. Clin Chem 2008; 54:249-255). The Malmö Diet and Cancer Study (MDCS) is a prospective cohort that enrolled participants between the ages of 44 and 73 years in Malmö, Sweden, starting in 1991. (Bergiund G, Eimsthl S, Janzon L, Larsson S A. The Malmö Diet and Cancer Study: design and feasibility. J Intern Med 1993; 233:45-51). In this study, participants with prevalent coronary disease at baseline were excluded. The BioImage Study enrolled asymptomatic participants between the ages of 55 and 80 years who were at risk for cardiovascular disease, beginning in 2008. This study included quantification of subclinical coronary artery disease in Agatston units, a metric that combines the area and density of observed coronary-artery calcification. (Baber U, Mehran R, Sartori S, et al. Prevalence, impact, and predictive value of detecting subclinical coronary and carotid atherosclerosis in asymptomatic adults: the BioImage study. J Am Coll Cardiol 2015; 65:1065-1074).

### Polygenic Risk Score

Applicant derived a polygenic risk score from an analysis of up to 50 single-nucleotide polymorphisms (SNPs) that had achieved genomewide significance for association with coronary artery disease in previous studies. Details regarding the cohort-specific genotyping platform and risk scores are provided in Table 12 in the, available with the full text of this article at NEJM.org. www.nejm.org/doi/full/10.1056/NEJMoa1605086—ref11 (Erdmann 3, Grosshennig A, Braund P S, et al. New susceptibility locus for coronary artery disease on chromosome 3q22.3. Nat Genet 2009; 41:280-282; Coronary Artery Disease (C4D) Genetics Consortium. A genome-wide association study in Europeans and South Asians identifies five new loci for coronary artery disease. Nat Genet 2011; 43:339-344; IBC 50K CAD Consortium. Large-scale gene-centric analysis identifies novel variants for coronary artery disease. PLoS Genet 2011; 7:e1002260-e1002260; The CARDIoGRAMplusC4D Consortium. Large-scale association analysis identifies new risk loci for coronary artery disease. Nat Genet 2013; 45:25-33). An example of the calculation of the polygenic risk score is provided in Table 13. Individual participant scores were created by adding up the number of risk alleles at each SNP and then multiplying the sum by the literature-based effect size. www.nejm.org/doi/full/10.1056/NEJMoa1605086—ref17 (Ripatti S, Tikkanen E, Orho-Mielander M, et al. A multilocus genetic risk score for coronary heart disease: case-control and prospective cohort analyses. Lancet 2010; 376:1393-1400). The genetic substructure of the population was assessed by calculating the principal components of ancestry. (Price A L, Patterson N J, Plenge R M, Weinbiatt M E, Shadick N A, Reich D. Principal components analysis corrects for stratification in genome-wide association studies. Nat Genet 2006; 38:904-909).

TABLE 12
Components of the genetic risk score by study. For SNPs not available by direct genotyping, a proxy (r2) is displayed. If no adequate (r2. 0.8)
proxy was available, N/A is displayed. The risk allele refers to the positive strand genotype for the Women's Genome Health Study (WGHS)/BioImage
studies and Malmo Diet and Cancer Study (MDCS) for SNPs unavailable in these cohorts. Participants missing more than two SNPs were excluded from
analysis; for the remainder, missing values were imputed to the population mean. Genotyping was performed using the Affymetrix 6.0 array (Affymetrix,
Santa Clara, California) for the Atherosclerosis Risk in Communities (ARIC) study, the Illumina HumanExome BeadChip v1.0 (Illumina, San Diego,
California) in WGHS, a previously reported multiplex method in MDCS, and the Illumina HumanExome Bead-Chip Array v1.1.

| | | Lead | ARIC | WGHS | MDCS | BioImage | | Risk | |
|---|---|---|---|---|---|---|---|---|---|
| | | SNP | Proxy | Proxy | Proxy | Proxy | Risk | Estimate | |
| Locus | Gene | (Literature) | (r²) | (r²) | (r²) | (r²) | Allele | (published) | Reference |
| 1p13.3 | SORT1 | rs599839 | rs629301 | | rs646776 | | A | 1.11 | Cardiogram |
| | | | (0.90) | | (0.91) | | | | Consortium |
| | | | | | | | | | (2011) |
| 1p32.2 | PPAP2B | rs17114036 | rs6588635 | | | | T | 1.11 | CARDIoGRAMplusC4D |
| | | | (0.83) | | | | | | Consortium |
| | | | | | | | | | (2013) |
| 1p32.3 | PCSK9 | rs11206510 | | | | | A | 1.08 | Cardiogram |
| | | | | | | | | | Consortium |
| | | | | | | | | | (2011) |
| 1q21.3 | IL6R | rs4845625 | rs6694817 | | | | A | 1.04 | CARDIoGRAMplusC4D |
| | | | (0.81) | | | | | | Consortium |
| | | | | | | | | | (2013) |
| 1q41 | MEA3 | rs17465637 | | | | | G | 1.14 | Cardiogram |
| | | | | | | | | | Consortium |
| | | | | | | | | | (2011) |
| 2p11.2 | GGCX/ | rs1561198 | rs2028900 | | rs2028900 | | T | 1.05 | CARDIoGRAMplusC4D |
| | VAMP8 | | (0.93) | | (0.95) | | | | Consortium |
| | | | | | | | | | (2013) |
| 2p21 | ABCG8 | rs6544713 | N/A | | rs4299376 | | T | 1.06 | CARDIoGRAMplusC4D |
| | | | | | (1.0) | | | | Consortium |
| | | | | | | | | | (2013) |
| 2p24.1 | APOB | rs515135 | rs12714264 | | | | C | 1.08 | CARDIoGRAMplusC4D |
| | | | (0.80) | | | | | | Consortium |
| | | | | | | | | | (2013) |
| 2q22.3 | ZEB2- | rs2252641 | | | | | G | 1.04 | CARDIoGRAMplusC4D |
| | AC074093.1 | | | | | | | | Consortium |
| | | | | | | | | | (2013) |
| 2q33.1 | WDR12 | rs6725887 | | rs2351524 | | rs2351524 | T | 1.12 | CARDIoGRAMplusC4D |
| | | | | (0.95) | | (0.95) | | | Consortium |
| | | | | | | | | | (2013) |
| 3q22.3 | MRAS | rs9818870 | | | | | T | 1.07 | CARDIoGRAMplusC4D |
| | | | | | | | | | Consortium |
| | | | | | | | | | (2013) |
| 4q31.22 | EDNRA | rs1878406 | rs6841581 | N/A | | N/A | T | 1.06 | CARDIoGRAMplusC4D |
| | | | (0.94) | | | | | | Consortium |
| | | | | | | | | | (2013) |
| 4q32.1 | GUCY1A3 | rs7692387 | rs3796587 | | | | G | 1.06 | CARDIoGRAMplusC4D |
| | | | (1.00) | | | | | | Consortium |
| | | | | | | | | | (2013) |
| 5q31.1 | SLC22A4/ | rs273909 | N/A | | | | C | 1.09 | CARDIoGRAMplusC4D |
| | SLC22A5 | | | | | | | | Consortium |
| | | | | | | | | | (2013) |
| 6p21.2 | KCNK5 | rs10947789 | rs6918122 | | | | T | 1.06 | CARDIoGRAMplusC4D |
| | | | (0.90) | | | | | | Consortium |
| | | | | | | | | | (2013) |
| 6p21.31 | ANKS1A | rs17609940 | | rs12205331 | | rs12205331 | G | 1.07 | Cardiogram |
| | | | | (0.85) | | (0.85) | | | Consortium |
| | | | | | | | | | (2011) |
| 6p24.1 | PHACTR1 | rs12526453 | N/A | rs9369640 | | rs9369640 | A | 1.1 | Cardiogram |
| | | | | (0.90) | | (0.90) | | | Consortium |
| | | | | | | | | | (2011) |
| 6q23.2 | TCF21 | rs12190287 | | N/A | | N/A | C | 1.07 | CARDIoGRAMplusC4D |
| | | | | | | | | | Consortium |
| | | | | | | | | | (2013) |
| 6q25.3 | SLC22A3/ | rs2048327 | | | | | C | 1.06 | CARDIoGRAMplusC4D |
| | LPAL2/LPA | | | | | | | | Consortium |
| | | | | | | | | | (2013) |
| 6q25.3 | LPA | rs3798220 | N/A | | | | C | 1.51 | Cardiogram |
| | | | | | | | | | Consortium |
| | | | | | | | | | (2011) |
| 6q25.3 | LPA | rs10455872 | N/A | N/A | | N/A | C | 1.45 | IBC 50K |
| | | | | | | | | | CAD |
| | | | | | | | | | Consortium |
| | | | | | | | | | (2011) |
| 6q26 | PLG | rs4252120 | | | | | T | 1.06 | CARDIoGRAMplusC4D |
| | | | | | | | | | Consortium |
| | | | | | | | | | (2013) |
| 7p21.1 | HDAC9 | rs2023938 | rs10245779 | | rs11984041 | | C | 1.07 | CARDIoGRAMplusC4D |
| | | | (0.85) | | (0.86) | | | | Consortium |
| | | | | | | | | | (2013) |
| 7q22.3 | BCAP29 | rs10953541 | rs7785962 | | | | C | 1.08 | Coronary |
| | | | (1.00) | | | | | | Artery |
| | | | | | | | | | Disease |
| | | | | | | | | | (C40) |
| | | | | | | | | | Genetics |
| | | | | | | | | | Consortium |
| | | | | | | | | | (2011) |
| 7q32.2 | ZC3HC1 | rs11556924 | | | | | C | 1.09 | CARDIoGRAMplusC4D |
| | | | | | | | | | Consortium |
| | | | | | | | | | (2013) |
| 8q24.13 | TRIBl | rs2954029 | rs2980875 | | | | A | 1.04 | CARDIoGRAMplusC4D |
| | | | (1.00) | | | | | | Consortium |
| | | | | | | | | | (2013) |
| 9p21.3 | CDKN2BAS | rs3217992 | | | | | T | 1.16 | CARDIoGRAMplusC4D |
| | | | | | | | | | Consortium |
| | | | | | | | | | (2013) |
| 9p21.3 | CDKN2A | rs4977574 | | | | | G | 1.29 | Cardiogram |
| | | | | | | | | | Consortium |
| | | | | | | | | | (2011) |
| 9q34.2 | ABO | rs579459 | rs651007 | | | | G | 1.07 | CARDIoGRAMplusC4D |
| | | | (1.00) | | | | | | Consortium |
| | | | | | | | | | (2013) |
| 10p11.23 | KIAA1462 | rs2505083 | | | rs2487928 | | C | 1.06 | CARDIoGRAMplusC4D |
| | | | | | (0.88) | | | | Consortium |
| | | | | | | | | | (2013) |
| 10q11.21 | CXCL12 | rs2047009 | | N/A | | N/A | G | 1.05 | CARDIoGRAMplusC4D |
| | | | | | | | | | Consortium |
| | | | | | | | | | (2013) |
| 10q11.21 | CXCL12 | rs501120 | | | rs1746048 | | A | 1.07 | CARDIoGRAMplusC4D |
| | | | | | (1.0) | | | | Consortium |
| | | | | | | | | | (2013) |
| 10q23.31 | LIPA | rs2246833 | | rs2246942 | rs1412444 | rs2246942 | C | 1.06 | CARDIoGRAMplusC4D |
| | | | | (1.0) | (0.98) | (1.0) | | | Consortium |
| | | | | | | | | | (2013) |
| 10q24.32 | CYP17Al | rs12413409 | | | | | C | 1.12 | Cardiogram |
| | | | | | | | | | Consortium |
| | | | | | | | | | (2011) |
| 11q22.3 | PDGFD | rs974819 | rs2128739 | | rs11226029 | | T | 1.07 | CARDIoGRAMplusC4D |
| | | | (0.89) | | (1.0) | | | | Consortium |
| | | | | | | | | | (2013) |
| 11q23.3 | APOA5 | rs964184 | | | | | G | 1.13 | Cardiogram |
| | | | | | | | | | Consortium |
| | | | | | | | | | (2011) |
| 12q24.1 | HNF1A | rs2259816 | | | | | T | 1.08 | Erdmann et |
| | | | | | | | | | al. (2009) |
| 12q24.12 | SH2B3 | rs3184504 | N/A | | | | T | 1.07 | CARDIoGRAMplusC4D |
| | | | | | | | | | Consortium |
| | | | | | | | | | (2013) |
| 13ql2.3 | FLT1 | rs9319428 | N/A | | | | A | 1.05 | CARDIoGRAMplusC4D |
| | | | | | | | | | Consortium |
| | | | | | | | | | (2013) |
| 13q34 | COL4A1 | rs4773144 | | | | | C | 1.07 | CARDIoGRAMplusC4D |
| | | | | | | | | | Consortium |
| | | | | | | | | | (2013) |
| 13q34 | COL4A1/ | rs9515203 | | N/A | | N/A | T | 1.08 | CARDIoGRAMplusC4D |
| | COL4A2 | | | | | | | | Consortium |
| | | | | | | | | | (2013) |
| 14q32.2 | HHIPL1 | rs2895811 | N/A | | | | C | 1.06 | CARDIoGRAMplusC4D |
| | | | | | | | | | Consortium |
| | | | | | | | | | (2013) |
| 15q25.1 | ADAMTS7 | rs3825807 | rs1994016 | N/A | | | T | 1.08 | Cardiogram |
| | | | (0.87) | | | | | | Consortium |
| | | | | | | | | | (2011) |
| 15q25.1 | ADAMTS7 | rs7173743 | rs7168915 | | | | T | 1.07 | CARDIoGRAMplusC4D |
| | | | (0.93) | | | | | | Consortium |
| | | | | | | | | | (2013) |
| 15q26.1 | FURIN/ | rs17514846 | rs1894401 | | | | T | 1.05 | CARDIoGRAMplusC4D |
| | FES | | (0.90) | | | | | | Consortium |
| | | | | | | | | | (2013) |
| 17p.112 | RASDl | rs12936587 | rs12449964 | | | | G | 1.06 | CARDIoGRAMplusC4D |
| | | | (0.94) | | | | | | Consortium |
| | | | | | | | | | (2013) |
| 17p13.3 | SMG6 | rs216172 | rs7217226 | | | | C | 1.07 | Cardiogram |
| | | | (1.00) | | | | | | Consortium |
| | | | | | | | | | (2011) |
| 17q21.32 | UBE2Z | rs46522 | rs15563 | | rs318090 | | T | 1.06 | Cardiogram |
| | | | (0.94) | | (1.0) | | | | Consortium |
| | | | | | | | | | (2011) |
| 19p13.2 | LDLR | rs1122608 | | | | | C | 1.1 | CARDIoGRAMplusC4D |
| | | | | | | | | | Consortium |
| | | | | | | | | | (2013) |
| 21q22.11 | KCNE2 | rs9982601 | rs9305545 | | | | A | 1.13 | CARDIoGRAMplusC4D |
| | | | (0.87) | | | | | | Consortium |
| | | | | | | | | | (2013) |

TABLE 13
Example of genetic risk score calculation. The number of coronary artery disease risk alleles was multiplied by a weighted risk
estimate (natural logarithm of the published odds ratio) for each genetic variant. For example, the 2011 CARDIoGRAM Consortium
analysis noted that the ‘A’ allele of rs599839 at the SORT1 locus was associated with an odds ratio of 1.11 for coronary
artery disease. Th eweight of the variant is expressed as the natural logarithm of 1.11 (0.104) in calculated the genetic risk
score. The WGHS participant represented here harbored the risk allele on one of her two chromosomes. The contribution of this
variant to her risk score is thus 1*0.104 = 0.104. These values were summed across all variants. This WGHS study participant
harbored 48 of a possible 88 risk alleles, corresponding to a genetic risk score of 4.187 (90th percentile of the cohort).

| | | Load SNP | | Ln(Published Odds | | # of Risk Alleles * |
|---|---|---|---|---|---|---|
| Locus | Gene Locus | (Literature) | WGHS Proxy | Ratio) | # of Risk Alleles | Ln(OR) |
| 1p13.3 | SORT1 | rs599839 | | 0.104 | 1 | 0.104 |
| 1p32.2 | PPAP2B | rs17114036 | | 0.104 | 2 | 0.209 |
| 1p32.3 | PCSK9 | rs11206510 | | 0.077 | 2 | 0.154 |
| 1q21.3 | IL6R | rs4845625 | | 0.039 | 2 | 0.078 |
| 1q41 | MIA3 | rs17465637 | | 0.131 | 2 | 0.262 |
| 2p11.2 | GGCX/VAMP8 | rs1561198 | | 0.049 | 0 | 0 |
| 2p21 | ABCG8 | rs6544713 | | 0.058 | 0 | 0 |
| 2p24.1 | APOB | rs15135 | | 0.077 | 1 | 0.077 |
| 2q22.3 | ZEB2-AC074093.1 | rs2252641 | | 0.039 | 2 | 0.078 |
| 2q33.1 | WDR12 | rs6725887 | rs2351524 (0.95) | 0.113 | 2 | 0.227 |
| 3q22.3 | MRAS | rs9818870 | | 0.068 | 1 | 0.068 |
| 4q32.1 | GUCY1A3 | rs7692387 | | 0.058 | 2 | 0.117 |
| 5q31.1 | SLC22A4/SLC22A5 | rs273909 | | 0.086 | 0 | 0 |
| 6p21.2 | KCNK5 | rs10947789 | | 0.058 | 2 | 0.117 |
| 6p21.31 | ANKS1A | rs17609940 | rs12205331 (0.85) | 0.068 | 1 | 0.068 |
| 6p24.1 | PHACTR1 | rs12526453 | rs9369640 (0.90) | 0.095 | 0 | 0 |
| 6q25.3 | SLC22A3/LPAL2/LPA | rs2048327 | | 0.058 | 1 | 0.058 |
| 6q25.3 | LPA | rs3793220 | | 0.412 | 0 | 0 |
| 6q26 | PLG | rs4252120 | | 0.058 | 2 | 0.117 |
| 7p21.1 | HDAC9 | rs2023938 | | 0.068 | 0 | 0 |
| 7q22.3 | BCAP29 | rs10953541 | | 0.077 | 1 | 0.077 |
| 7q32.2 | ZC3HC1 | rs11556924 | | 0.086 | 1 | 0.086 |
| 8q24.13 | TRIB1 | rs2954029 | | 0.039 | 1 | 0.039 |
| 9p21.3 | CDKN2BAS | rs3217992 | | 0.148 | 2 | 0.297 |
| 9p21.3 | CDKN2A | rs4977574 | | 0.255 | 2 | 0.509 |
| 9q34.2 | ABO | rs579459 | | 0.068 | 0 | 0 |
| 10p11.23 | KIAA1462 | rs2505083 | | 0.058 | 0 | 0 |
| 10q11.21 | CXCL12 | rs501120 | | 0.068 | 1 | 0.068 |
| 10q23.31 | LIPA | rs2246833 | rs2246942 (1.0) | 0.058 | 0 | 0 |
| 10q24.32 | CYP17A1 | rs12413409 | | 0.113 | 2 | 0.227 |
| 11q22.3 | PDGFD | rs974819 | | 0.068 | 2 | 0.135 |
| 11q23.3 | APOA5 | rs964184 | | 0.122 | 2 | 0.244 |
| 12q24.1 | HNF1A | rs2259816 | | 0.077 | 1 | 0.077 |
| 12q24.12 | SH2B3 | rs3134504 | | 0.068 | 1 | 0.068 |
| 13q12.3 | FLT1 | rs9319428 | | 0.049 | 0 | 0 |
| 13q34 | COI4A1 | rs4773144 | | 0.068 | 0 | 0 |
| 14q32.2 | HHIPL1 | rs2895811 | | 0.058 | 0 | 0 |
| 15q25.1 | ADAMTS7 | rs7173743 | | 0.068 | 2 | 0.135 |
| 15q26.1 | FURIN/FES | rs17514846 | | 0.049 | 1 | 0.049 |
| 17p11.2 | RASD1 | rs12936587 | | 0.058 | 1 | 0.058 |
| 17p13.3 | SMG6 | rs216172 | | 0.068 | 2 | 0.135 |
| 17q21.32 | UBE2Z | rs46522 | | 0.058 | 1 | 0.058 |
| 19p13.2 | LDLR | rs1122608 | | 0.095 | 2 | 0.191 |
| 21q22.11 | KCNE2 | rs9932601 | | 0.122 | 0 | 0 |
| | | | | Total: | 48 | 4.187 |

Healthy Lifestyle Factors

Applicant adapted four healthy lifestyle factors from the strategic goals of the American Heart Association (AHA)—no current smoking, no obesity (body-mass index [the weight in kilograms divided by the square of the height in meters], <30), physical activity at least once weekly, and a healthy diet pattern. www.nejm.org/doi/full/10.1056/NEJMoa16O5O86—ref31 (Lloyd-Jones D M, H-long Y, Labarthe D, et al. Defining and setting national goals for cardiovascular health promotion and disease reduction: the American Heart Association's strategic Impact Goal through 2020 and beyond. Circulation 2010; 121:586-613). A healthy diet pattern was ascertained on the basis of adherence to at least half of the following recently endorsed characteristics (Mozaffarian D. Dietary and policy priorities for cardiovascular disease, diabetes, and obesity: a comprehensive review. Circulation 2016; 133:187-225): consumption of an increased amount of fruits, nuts, vegetables, whole grains, fish, and dairy products and a reduced amount of refined grains, processed meats, unprocessed red meats, sugar-sweetened beverages, trans fats (WGHS only), and sodium (WGHS only). Because a detailed food-frequency questionnaire was not performed in the BioImage Study, diet scores in that cohort focused on self-reported consumption of fruits, vegetables, and fish. Additional details regarding cohort-specific metrics for lifestyle factors are provided in Table S3.

TABLE 14
Healthy lifestyle factor criteria by study population

| | Atherosclerosis Risk in | Women's Genome | Maimö Diet and | |
|---|---|---|---|---|
| | Communities | Health Study | Cancer Study | Bioimage Study |
| Absence of | Baseline survey self-report | Baseline survey self-report | Baseline survey self-report | Baseline survey self-report |
| Current Smoking | | | | |
| Absence of Obesity | BMI <30 kg/m² at baseline | BMI <30 kg/m² via self- | BMI <30 kg/m² at baseline | BMI <30 kg/m² via self- |
| | examination | reported height and weight | examination | reported height and weight |
| Regular Physical Activity | Self-reported physical | Self-reported strenuous | Self-reported strenuous | Self-reported moderate |
| | activity ≥once/week | physical activity ≥once/week | physical activity ≥once/week | physical activity ≥5 times/ |
| | | | | week or vigorous |
| | | | | activity ≥once/week |
| Healthy Diet | At least 5 of the following | At least 6 of the following | At least 5 of the following | At least 2 of the following |
| | 10 characteristics, as | 12 characteristics, as | 10 charactertics, as | three characteristics |
| | assessed by food | assessed by food | assessed by food | assessed by baseline |
| | frequency questionnaire: | frequency questionnaire: | frequency questionnaire, | survey: |
| | 1. Fruits: ≥3 servings/day | 1. Fruits: ≥3 servings/day | diet record and structured | 1. Fruits: ≥3 servings/day |
| | 2. Nuts: ≥1 serving/week | 2. Nuts: ≥1 serving/week | interview: | 2. Vegetables: ≥5 times/ |
| | 3. Vegetables: ≥3 | 3. Vegetables: ≥3 | 1. Fruits: ≥3 servings/day | week |
| | servings/day | servings/day | 2. Nuts: ≥1 serving/week | 3. Fish: ≥3 times/week |
| | 4. Whole grains: ≥3 | 4. Whole grains: ≥3 | 3. Vegetables: ≥3 | |
| | servings/day | servings/day | servings/day | |
| | 5. Fish: ≥2 servings/week; | 5. Fish: ≥2 servings/week; | 4. Whole grains: ≥3 | |
| | 6. Dairy: ≥2.5 servings/day | 6. Dairy: ≥2.5 servings/day | servings/day | |
| | 7. Refined grains: ≤1.5 | 7. Refined grains: ≤1.5 | 5. Fish: ≥2 servings/week; | |
| | servings/day | servings/day | 6. Dairy: ≥2.5 servings/day | |
| | 8. Processed meats: ≤1 | 8. Processed meats: ≤1 | 7. Refined grains: ≤1.5 | |
| | serving/week | serving/week | servings/day | |
| | 9. Unprocessed red meats | 9. Unprocessed red meats | 8. Processed meats: ≤1 | |
| | ≤1.5 servings/week | ≤1.5 servings/week | serving/week | |
| | 10. Sugar-sweetened | 10. Trans fat: ≤cohort | 9. Unprocessed red meats | |
| | beverages: ≤1 | median | ≤1.5 servings/week | |
| | serving/week | 11. Sugar-sweetened | 10. Sugare-seetened | |
| | | beverages: ≤1 servings/week | beverages: ≤1 serving/week | |
| | | 12. Sodium: ≤2000 mg | | |

### Study End Points

The primary study end point for the prospective cohort populations was a composite of coronary artery disease events that included myocardial infarction, coronary revascularization, and death from coronary causes. End-point adjudication was performed by a committee review of medical records within each cohort. In the BioImage Study, a cross-sectional analysis of baseline scores for coronary-artery calcification was performed.

### Statistical Analysis

Applicant used Cox proportional-hazard models to test the association of genetic and lifestyle factors with incident coronary events. Applicant compared hazard ratios for participants at high genetic risk (i.e., highest quintile of polygenic scores) with those at intermediate risk (quintiles 2 to 4) or low risk (lowest quintile), as described previously. www.nejm.org/doi/full/10.1056/NEJMoa1605086—ref22 (Mega J L, Stitziei N O, Smith J G, et al. Genetic risk, coronary heart disease events, and the clinical benefit of statin therapy: an analysis of primary and secondary prevention trials. Lancet 2015; 385:2264-2271; Tada H, Melander O, Louie J Z, et al. Risk prediction by genetic risk scores for coronary heart disease is independent of self-reported family history. Eur Heart J 2016; 37:561-567). Similarly, Applicant compared a favorable lifestyle (which was defined as the presence of at least three of the four healthy lifestyle factors) with an intermediate lifestyle (two healthy lifestyle factors) or an unfavorable lifestyle (no or only one healthy lifestyle factor). The primary analyses included adjustment for age, sex, self-reported education level, and the first five principal components of ancestry (unavailable in MDCS). In addition, WGHS analyses were adjusted for initial trial randomization to aspirin versus placebo and vitamin E versus placebo. Applicant used Cox regression to calculate 10-year event rates, which were standardized to the mean of all predictor variables within each population. Because of a skewed distribution of scores for coronary-artery calcification in the BioImage Study, linear regression was performed on natural log-transformed calcification scores with an offset of 1. Predicted values were then reverse-transformed to calculate standardized scores, with higher values indicating an increased burden of coronary atherosclerosis. All the analyses were performed with the use of R software, version 3.1 (R Project for Statistical Computing).

### Results

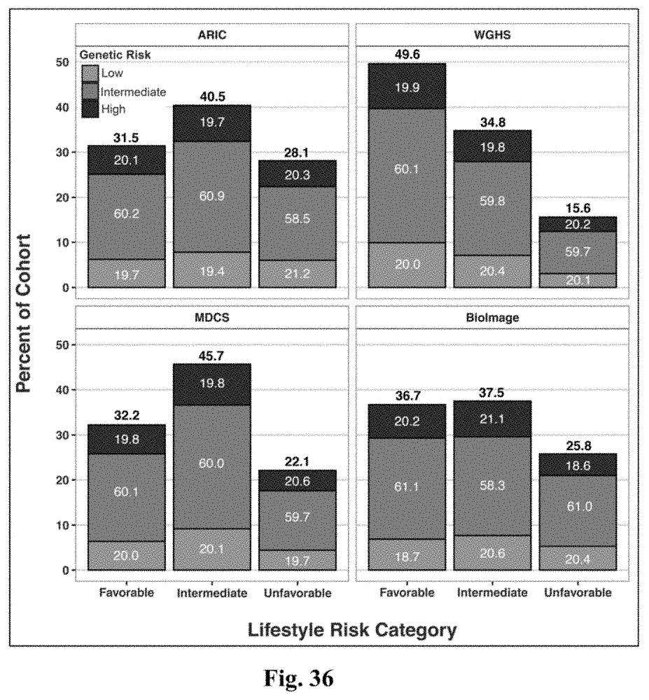

The populations in the prospective cohort studies included 7814 of 11,478 white participants in the ARIC cohort, 21,222 of 23,294 white women in the WGHS cohort, and 22,389 of 30,446 participants in the MDCS cohort for whom genotype and covariate data were available (Table 1) Characteristics of the Participants at Baseline.). During follow-up, 1230 coronary events were observed in the ARIC cohort (median follow-up, 18.8 years), 971 coronary events in the WGHS cohort (median follow-up, 20.5 years), and 2902 coronary events in the MDCS cohort (median follow-up, 19.4 years) (Table 15). Categories of genetic and lifestyle risk were mutually independent within each cohort (FIG. 36).

TABLE 15
Number of each component of the composite coronary endpoint
within the prospective cohorts

| | | Women's | Malmö Diet |
|---|---|---|---|
| | Atherosclerosis | Genome | and |
| | Risk in | Health | Cancer |
| | Communities | Study | Study |
| Composite Coronary Endpoint | 1,230 | 971 | 2,902 |
| Myocardial Infarction | 602 | 368 | 1,444 |
| Coronary Revascularization | 568 | 589 | 1,226 |
| Death From Coronary Causes | 60 | 14 | 232 |

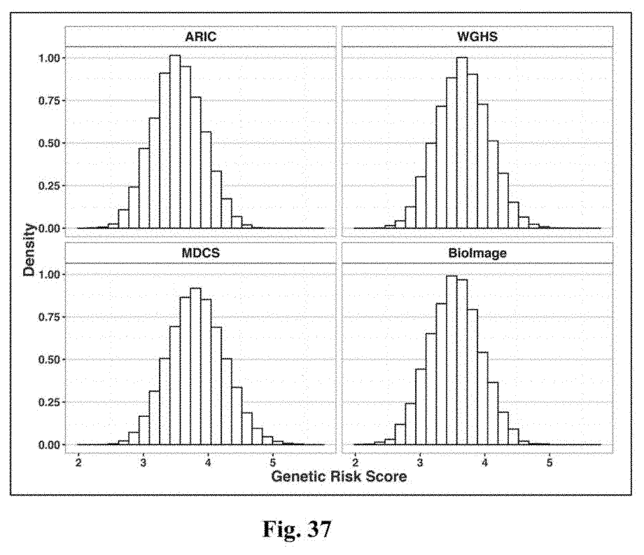

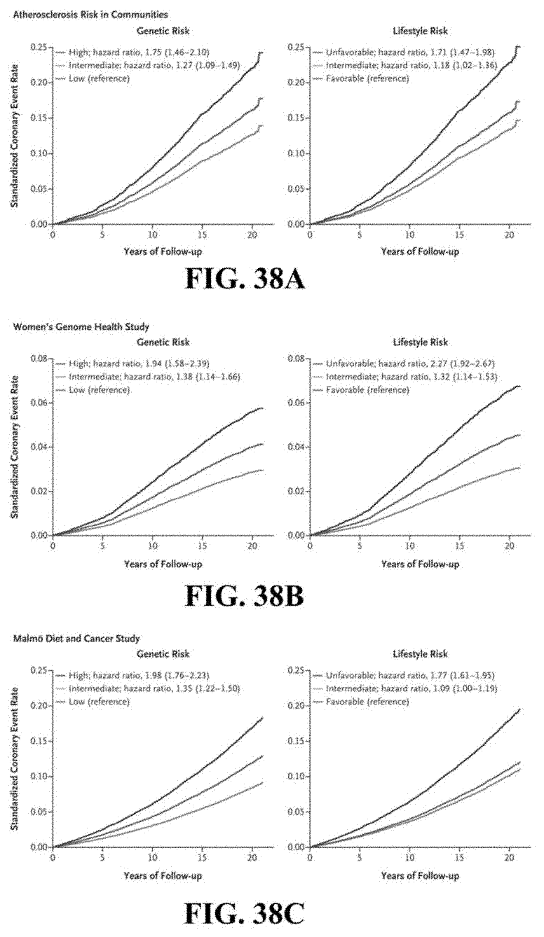

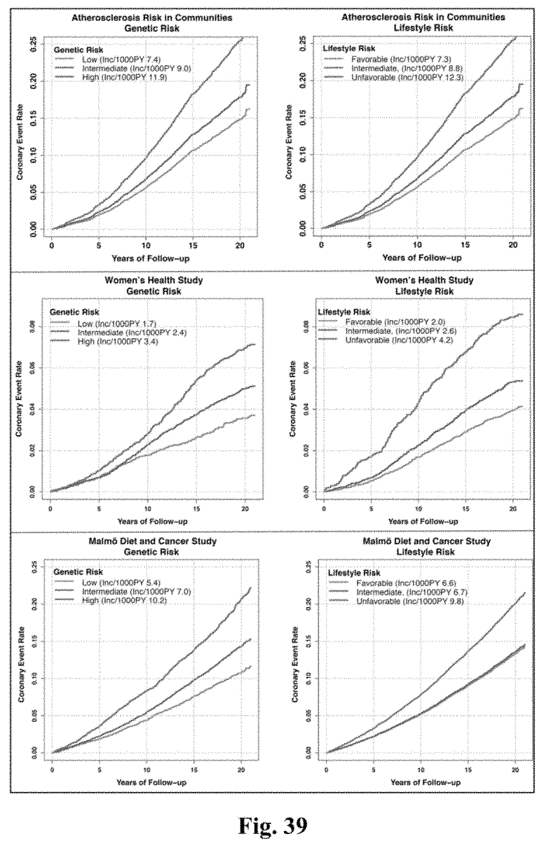

Polygenic risk scores approximated a normal distribution within each cohort (FIG. 37). A risk gradient was noted across quintiles of genetic risk such that the participants at high genetic risk (i.e., in the top quintile of the polygenic scores) were at significantly higher risk of coronary events than those at low genetic risk (i.e., in the lowest quintile), with adjusted hazard ratios of 1.75 (95% confidence interval [CI], 1.46 to 2.10) in the ARIC cohort, 1.94 (95% CI, 1.58 to 2.39) in the WGHS cohort, and 1.98 (95% CI, 1.76 to 2.23) in the MDCS cohort (FIG. 38) Standardized Coronary Events Rates. According to Genetic and Lifestyle Risk in the Prospective Cohorts., and Table 16 and FIG. 39). Across all three cohorts, the relative risk of incident coronary events was 91% higher among participants at high genetic risk than among those at low genetic risk (hazard ratio, 1.91; 95% CI, 1.75 to 2.09). A family history of coronary artery disease was an imperfect surrogate for genotype-defined risk, although the prevalence of such a self-reported family history tended to be higher among participants at high genetic risk than among those at low genetic risk. Levels of low-density lipoprotein (LDL) cholesterol were modestly increased across categories of genetic risk within each cohort. By contrast, genetic risk categories were independent of other cardiometabolic risk factors and 10-year cardiovascular risk as predicted by the pooled cohorts equation of the American College of Cardiology-AHA (Tables 17-20).

TABLE 16
Risk of coronary events according to genetic risk score quintiles. Cox
regression models were adjusted for age, gender (in ARIC and MDCS), randomization to
Vitamin E or aspirin (in WGHS), education level, and principal components of ancestry (in
ARIC and WGHS). Cohort-specific findings were combined using random effects meta-analysis.
Those in the lowest quintile of genetic risk serve as the reference group. Values displayed
represent hazard ratios and 95% confidence intervals.

| | Atherosclerosis | | | |
|---|---|---|---|---|
| | Risk in | Women's Genome | Malmo Diet and | |
| | Communities | Health Study | Cancer Study | Combined |
| Genetic risk | | | | |
| Category | | | | |
| Quintile 1 | Reference | Reference | Reference | Reference |
| Quintile 2 | 1.16 (0.96-1.40) | 1.20 (0.83-0.96) | 1.26 (1.11-1.43) | 1.22 (1.11-1.34) |
| Quintile 3 | 1.26 (1.04-1.52) | 1.40 (1.13-1.74) | 1.28 (1.13-1.45) | 1.30 (1.18-1.42) |
| Quintile 4 | 1.41 (1.17-1.69) | 1.53 (1.23-1.89) | 1.53 (1.35-1.73) | 1.50 (1.36-1.64) |
| Quintile 5 | 1.75 (1.46-2.10) | 1.94 (1.58-2.39) | 1.98 (1.76-2.23) | 1.91 (1.75-2.09) |
| P-Trend | 8.1 × 10⁻¹¹ | 7.4 × 10⁻¹² | 3.2 × 10⁻³³ | |

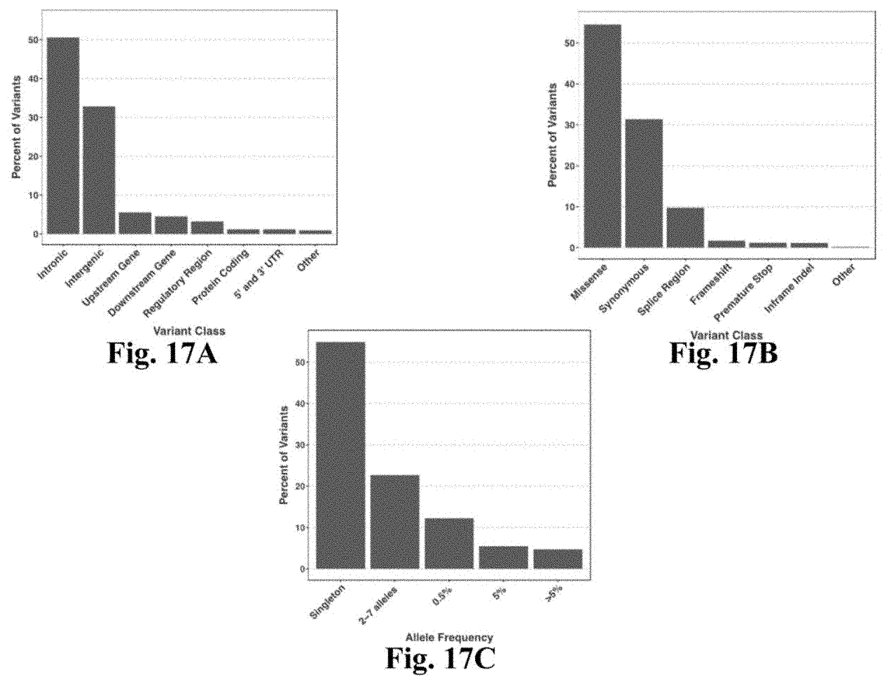

FIG. 17. Baseline characteristics by genetic risk category, ARIC. Values represent N (% with recorded values), mean (SD), or median (IQR). P-values computed via ANOVA for continuous variables (TG modeled using Kruskal-Wallis test) and chi-square test for categorical variables. FH (family history); CAD (coronary artery disease). Family history of premature coronary artery disease refers to self-reported parental history of myocardial infarction prior to age 60 years.

| | Low Risk | | Intermediate Risk | | High Risk | | |
|---|---|---|---|---|---|---|---|
| | N = 1,563 | | N = 4,688 | | N = 1,563 | | P-value |
| Age, years | 54 | (5.7) | 54 | (5.6) | 54 | (5.7) | 0.09 |
| Male Gender | 739 | (47%) | 2,105 | (45%) | 711 | (45%) | 0.26 |
| History of Hypertension | 405 | (26%) | 1,218 | (26%) | 397 | (25%) | 0.88 |
| History of Diabetes Mellitus | 140 | (9%) | 349 | (7%) | 143 | (9%) | 0.04 |
| Family History of Premature CAD | 143 | (11%) | 439 | (11%) | 169 | (13%) | 0.14 |
| Body-mass index, kg/m² | 27 | (5.0) | 27 | (4.8) | 27 | (4.8) | 0.21 |
| Lipid Levels | | | | | | | |
| LDL Cholesterol, mg/dl | 134 | (37) | 137 | (38) | 139 | (37) | <0.001 |
| HDL Cholesterol, mg/dl | 38 | (11) | 37 | (11) | 37 | (10) | 0.07 |
| Triglycerides, mg/dl | 112 | (80-59) | 113 | (81-162) | 117 | (82-165) | 0.11 |
| Lipid-lowering Medication | 6 | (0.4%) | 26 | (0.6%) | 13 | (0.8%) | 0.24 |
| Healthy Lifestyle Factors | | | | | | | |
| No Current Smoking | 1,156 | (74%) | 3,864 | (76%) | 1,163 | (74%) | 0.25 |
| Nonobese | 1,198 | (77%) | 3,665 | (78%) | 1,230 | (79%) | 0.33 |
| Regular Physical Activity | 547 | (35%) | 1,659 | (35%) | 537 | (34%) | 0.76 |
| Healthy Diet | 303 | (19%) | 901 | (19%) | 311 | (20%) | 0.84 |
| Lifestyle Risk Category | | | | | | | |
| 3-4 Healthy Lifestyle Factors | 484 | (31%) | 1,480 | (32%) | 495 | (32%) | |
| 2 Healthy Lifestyle Factors | 613 | (39%) | 1,926 | (41%) | 623 | (40%) | 0.41 |
| 0-1 Healthy Lifestyle Factors | 466 | (30%) | 1,282 | (27%) | 445 | (28%) | |

TABLE 18
Baseline characteristics by genetic risk category, WGHS. Values represent N (% with recorded values),
mean (SD), or median (IQR). P-values computed via ANOVA for continuous variables
(TG modeled using Kruskal-Wallis test) and chi-square test for categorical variables.
FH (family history); CAD (coronary artery disease). Family history of premature coronary
artery disease refers to self-reported parental history of myocardial infarction prior to age 60 years.

| | Low Risk | | Intermediate Risk | | High Risk | | |
|---|---|---|---|---|---|---|---|
| | N = 4,280 | | N = 12,716 | | N = 4,226 | | P-value |
| Age, years | 54.2 | (7.2) | 54.2 | (7.1) | 54.1 | (6.9) | 0.25 |
| History of Hypertension | 1,038 | (24%) | 3,080 | (24) | 1,046 | (25%) | 0.78 |
| History of Diabetes Mellitus | 105 | (3%) | 313 | (3%) | 101 | (2%) | 0.97 |
| FH of Premature CAD | 420 | (11%) | 1,472 | (13%) | 584 | (16%) | <0.001 |
| Body-mass Index, kg/m² | 25.9 | (4.8) | 25.9 | (5) | 25.9 | (5) | 0.83 |
| Lipid Levels | | | | | | | |
| LDL Cholesterol, mg/dl | 121 | (34) | 124 | (34) | 126 | (34) | <0.001 |
| HDL Cholesterol, mg/dl | 54 | (15) | 54 | (15) | 54 | (15) | 0.45 |
| Triglycerides, mg/dl | 118 | (84-172) | 120 | (84-176) | 119 | (84-177) | 0.85 |
| Lipid-lowering Medication | 129 | (3%) | 406 | (3%) | 155 | (3.7%) | 021 |
| C-Reactive Protein | 2.0 | (0.8-4.4) | 2.0 | (0.8-4.4) | 1.9 | (0.8-4.3) | 0.37 |
| Healthy Lifestyle Factors | | | | | | | |
| No Current Smoking | 3,751 | (88%) | 11,298 | (89%) | 3,735 | (88%) | 0.10 |
| Nonobese | 3,551 | (83%) | 10,535 | (83%) | 3,480 | (82%) | 0.70 |
| Regular Physical Activity | 1,872 | (44%) | 5,556 | (44%) | 1,828 | (43%) | 0.87 |
| Healthy Diet | 1,460 | (34%) | 4,328 | (34%) | 1,463 | (35%) | 0.78 |
| Lifestyle Risk Category | | | | | | | |
| 3-4 Healthy Lifestyle Factors | 2,103 | (49%) | 6,319 | (50%) | 2,094 | (50%) | |
| 2 Healthy Lifestyle Factors | 1,509 | (35%) | 4,414 | (35%) | 1,462 | (35%) | 0.95 |
| 0-1 Healthy Lifestyle Factors | 668 | (16%) | 1,983 | (16%) | 670 | (16%) | |

TABLE 19
Baseline characteristics by genetic risk category, MDCS. Values represent N
(% with recorded values), mean (SD), or median (IQR). P-values computed via ANOVA for
continuous variables (TG modeled using Kruskal-Wallis test) and chi-square test for categorical
variables. FH (family history); CAD (coronary artery disease). Family history of premature
coronary artery disease refers to self-reported parental history of myocardial infarction. *P-value
for test of linear trend = 0.12.

| | Low Risk | | Intermediate Risk | | High Risk | | |
|---|---|---|---|---|---|---|---|
| | N = 4,478 | | N = 13,434 | | N = 4,477 | | P-value |
| Age, years | 58.2 | (7.8) | 58.0 | (7.7) | 57.8 | (7.7) | 0.11 |
| Male gender | 1,733 | (39%) | 5,061 | (38%) | 1,721 | (38%) | 0.39 |
| History of Hypertension | 2,732 | (61%) | 8,018 | (60%) | 2,803 | (63%) | 0.002* |
| History of Diabetes Mellitus | 175 | (4%) | 557 | (4%) | 172 | (4%) | 0.59 |
| FH of CAD | 1,267 | (28%) | 4,352 | (32%) | 1,606 | (36%) | <0.0001 |
| Body-mass Index, kg/m² | 25.7 | (3.9) | 25.7 | (3.9) | 25.7 | (4.0) | 0.70 |
| Lipid Levels | | | | | | | |
| LDL Cholesterol, mg/dl | 157 | (38) | 161 | (38) | 167 | (39) | <0.0001 |
| HDL Cholesterol, mg/dl | 54 | (15) | 54 | (15) | 53 | (15) | 0.84 |
| Triglycerides, mg/dl | 101 | (76-143) | 102 | (75-1.39) | 105 | (79-152) | 0.08 |
| Lipid-lomiering Medication | 79 | (2%) | 290 | (2%) | 119 | (3%) | 0.02 |
| C-Reactive Protein, mg/L | 1.4 | (0.7-2.8) | 1.4 | (0.6-2.7) | 1.3 | (0.6-2.6) | 0.17 |
| Healthy Lifestyle Factors | | | | | | | |
| No Current Smoking | 3,214 | (72%) | 9,703 | (72%) | 3,245 | (72%) | 0.75 |
| Nonobese | 3,891 | (87%) | 11,716 | (87%) | 3,900 | (87%) | 0.86 |
| Regular Physical Activity | 1,861 | (42%) | 5,470 | (41%) | 1,762 | (39%) | 0.10 |
| Healthy Diet | 578 | (13%) | 1,660 | (12%) | 557 | (12%) | 0.62 |
| Lifestyle Risk Category | | | | | | | |
| 3-4 Healthy Lifestyle Factors | 1,444 | (32%) | 4,336 | (32%) | 1,430 | (32%) | |
| 2 Healthy Lifestyle Factors | 2,060 | (46%) | 6,145 | (46%) | 2,029 | (45%) | 0.82 |
| 0-1 Healthy lifestyle Factors | 974 | (22%) | 2,953 | (22%) | 1,018 | (23%) | |

TABLE 20
ACC/AHA 2013 Atherosclerotic Cardiovascular Disease Risk Score According to Genetic
Risk Categories. Ten-year predicted risk according to the ACC/AHA Pooled Cohorts
Equation was determined within each category of genetic risk. Indivisuals reporting baseline
use of lipid-lowering therapy were excluded from this analysis. The Malmo Diet and Cancer
Study calculations were restricted to individuals with baseline total and HDL cholesterol
values available (N = 4,172). Values displayed represent mean (standard deviation).

| | | | |
|---|---|---|---|
| Atherosclerosis Risk | Women's Genome | Malmö Diet and | |
| in Communities | Health Study | Cancer Study | Bioimage Study |

Genetic Risk Category

| | | | | |
|---|---|---|---|---|
| Low Risk | 9.9 (10.8) | 3.5 (4.2) | 9.8 (8.4) | 17.6 (11.7) |
| Intermediate Risk | 9.2 (10.6) | 3.6 (4.4) | 9.5 (8.0) | 18.7 (12.3) |
| High Risk | 9.8 (11.6) | 3.5 (4.2) | 10.2 (8.6) | 17.7 (10.9) |
| P-Trend | 0.62 | 0.91 | 0.12 | 0.91 |

TABLE 21
Association of healthy lifestyle factors with incident coronary events. Cox
regression models were adjusted for age, gender (in ARIC and MDCS), randomization to
Vitamin E or aspirin (in WGHS), education level, and principal components of ancestry (in
ARIC and WGHS). Cohort-specific findings were combined using random effects meta-analysis.
Hazard ratios, 95% confidence intervals and P-values are displayed within each cell.

| | Atherosclerosis Risk | Women's Genome | Malmö Diet and | |
|---|---|---|---|---|
| Heaithy Lifestyle Factor | in Communities | Health Study | Cancer Study | Combined |
| No Current Smoking | 0.64 | 0.45 | 0.58 | 0.56 |
| | (0.57-0.73) | (0.38-0.53) | (0.53-0.62) | (0.47-0.66) |
| | <0.001 | <0.001 | <0.001 | <0.001 |
| Non-obese | 0.67 | 0.58 | 0.74 | 0.66 |
| | (0.59-0.76) | (0.50-0.68) | (0.67-0.81) | (0.58-0.76) |
| | <0.001 | <0.001 | <0.001 | <0.001 |
| Regular Physical Activity | 0.91 | 0.78 | 0.92 | 0.88 |
| | (0.80-1.03) | (0.69-0.89) | (0.86-0.99) | (0.80-0.97) |
| | 0.12 | <0.001 | 0.035 | 0.007 |
| Healthy Diet | 0.93 | 0.83 | 0.96 | 0.91 |
| | (0.79-1.08) | (0.73-0.95) | (0.86-1.08) | (0.93-0.99) |
| | 0.34 | 0.008 | 0.54 | 0.036 |

TABLE 22
Risk of coronary events according to number of healthy lifestyle factors.
Cox regression models were adjusted for age, gender (in ARIC and MDCS), randomization to
Vitamin E or aspirin (in WGHS), education level, and principal components of ancestry (in
ARIC and WGHS). Cohort-specific findings were combined using random effects meta-analysis.
Those adherent to all four healthy lifestyle factors serve as the reference group. Values displayed
represent hazard ratios and 95% confidence intervals.

| | Atherosclerosis | Women's | | |
|---|---|---|---|---|
| | Risk in | Genome | Malmö Diet and | |
| | Communities | Health Study | Cancer Study | Combined |
| Lifestyle Risk Category | | | | |
| 4 Healthy Lifestyle Factors | Reference | Reference | Reference | Reference |
| 3 Healthy Lifestyle Factors | 1.42 (1.05-1.90) | 1.07 (0.86-1.33) | 0.96 (0.78-1.18) | 1.11 (0.78-1.18) |
| 2 Healthy Lifestyle Factors | 1.56 (1.17-2.08) | 1.39 (1.13-1.71) | 1.05 (0.86-1.29) | 1.29 (1.03-1.63) |
| 1 Healthy Lifestyle Factor | 2.17 (1.62-2.90) | 2.17 (1.73-2.72) | 1.62 (1.32-2.00) | 1.93 (1.57-2.38) |
| 0 Healthy Lifestyle Factors | 3.30 (2.25-4.82) | 5.32 (3.66-7.72) | 3.00 (2.25-4.00) | 3.40 (2.62-4.42) |
| P-Trend | 7.6 × 10⁻¹⁵ | 6.7 × 10⁻²¹ | 3.0 × 10⁻²⁹ | |

Each cohort was divided into three lifestyle risk categories: favorable (at least three of the four healthy lifestyle factors), intermediate (two healthy lifestyle factors), or unfavorable (no or only one healthy lifestyle factor). Participants with an unfavorable lifestyle had higher rates of baseline hypertension and diabetes, a higher body-mass index, and less favorable levels of circulating lipids than did those with a favorable lifestyle (Tables 23, 24, and 25). An unfavorable lifestyle was associated with a higher risk of coronary events than a favorable lifestyle, with an adjusted hazard ratio of 1.71 (95% CI, 1.47 to 1.98) in the ARIC cohort, 2.27 (95% CI, 1.92 to 2.67) in the WGHS cohort, and 1.77 (95% CI, 1.61 to 1.95) in the MDCS cohort (FIG. 38, and FIG. 38).

TABLE 23
Baseline characteristics by lifestyle risk category, AMC Values represent N (% with
recorded values), mean (SD), or median (IQR). P-values computed via ANOVA for continuous
variables (TG modeled using Kruskal-Wallis test) and chi-square test for categorical variables.
FH (family history); CAD (coronary artery disease). Family history of premature coronary
artery disease refers to self-reported parental history of myocardial infarction prior to age 60 years.

| | Favorable | | Intermediate | | Unfavorable | | |
|---|---|---|---|---|---|---|---|
| | Lifestyle | | Lifestyle | | Lifestyle | | |
| | N = 2,459 | | N = 3,162 | | N = 2,193 | | P-value |
| Age, years | 55 | (5.8) | 54 | (5.6) | 54 | (5.6) | <0.001 |
| Male Sex | 1,100 | (45%) | 1,453 | (46%) | 1,002 | (46%) | 0.65 |
| History of Hypertension | 548 | (22%) | 822 | (26%) | 650 | (30%) | <0.001 |
| History of Diabetes Mellitus | 148 | (6%) | 241 | (8%) | 243 | (11%) | <0.001 |
| Family History of Premature CAD | 228 | (11%) | 296 | (11%) | 227 | (12%) | 0.23 |
| Body-mass Index, kg/m² | 25.3 | (3.2) | 26.6 | (4.3) | 29.3 | (6.0) | <0.001 |
| Lipid Levels | | | | | | | |
| LDL Cholesterol, mg/dl | 134 | (37) | 136 | (37) | 140 | (38) | <0.001 |
| HDL Cholesterol, mg/dl | 39 | (11) | 37 | (11) | 34 | (10) | <0.001 |
| Triglycerides, mg/dl | 102 | (73-147) | 112 | (81-160) | 129 | (95-177) | <0.001 |
| Lipid-lowering Medication | 17 | (0.7%) | 18 | (0.6%) | 10 | (0.5%) | 0.57 |
| Healthy Lifestyle Factors | | | | | | | |
| No Current Smoking | 2,384 | (97%) | 2,661 | (84%) | 828 | (38%) | <0.001 |
| Non-obese | 2364 | (96%) | 2,657 | (84%) | 1,072 | (49%) | <0.001 |
| Regular Physical Activity | 2,003 | (81%) | 691 | (22%) | 49 | (2%) | <0.001 |
| Healthy Diet | 1,166 | (47%) | 315 | (10%) | 34 | (2%) | <0.001 |
| Genetic Risk Category | | | | | | | |
| Low Genetic Risk | 484 | (20%) | 613 | (19%) | 466 | (21%) | |
| Intermediate Genetic Risk | 1,480 | (60%) | 1,926 | (61%) | 1,282 | (58%) | 0.41 |
| High Genetic Risk | 495 | (20%) | 623 | (20%) | 445 | (20%) | |

TABLE 24
Baseline characteristics by lifestyle risk category, WGHS. Values represent
N (% with recorded values), mean (SD), or median (IQR). P-values computed via ANOVA for
continuous variables (T modeled using Kruskal-Wallis test) and chi-square test for categorical
variables. FH (family history); CAD (coronary artery disease). Family history of premature
coronary artery disease refers to self-reported parental history of myocardial infarction prior to
age 60 years.

| | Favorable | | Intermediate | | Unfavorable | | |
|---|---|---|---|---|---|---|---|
| | Lifestyle | | Lifestyle | | Lifestyle | | |
| | N = 10,516 | | N =7,385 | | N = 3,321 | | P-value |
| Age, years | 54.5 | (7.3) | 54.1 | (7.1) | 53.4 | (6.5%) | <0.001 |
| History of Hypertension | 2150 | (20%) | 1,850 | (25%) | 1464 | (35%) | <0.001 |
| History of Diabetes Mellitus | 178 | (2%) | 168 | (2%) | 173 | (5%) | <0.001 |
| FH of Premature CAD | 1194 | (13%) | 852 | (13%) | 430 | (15%) | 0.02 |
| Body-mass Index, kg/m² | 24.3 | (3.3) | 25.9 | (4.6) | 30.8 | (6.4) | <0.001 |
| Lipid Levels | | | | | | | |
| LDL Cholesterol, mg/dl | 122 | (34) | 125 | (34) | 129 | (35) | <0.001 |
| HDL Cholesterol, mg/dl | 57 | (15) | 53 | (15) | 47 | (13) | <0.001 |
| Triglycerides, mg/dl | 111 | (78-161) | 123 | (85-178) | 147 | (102-212) | <0.001 |
| Lipid-lowering Medication | 354 | (3%) | 232 | (3%) | 104 | (3%) | 0.63 |
| C-Reactive Protein | 1.6 | (0.6-3.4) | 2.1 | (0.9-4.4) | 3.8 | (1.8-6.8) | <0.001 |
| Healthy Lifestyle Factors | | | | | | | |
| No Current Smoking | 10,309 | (98%) | 6,674 | (90%) | 1,801 | (54%) | <0.001 |
| Nonobese | 10,164 | (97%) | 6,230 | (84%) | 1472 | (35%) | <0.001 |
| Regular Physical Activity | 8,148 | (78%) | 1,058 | (14%) | 50 | (2%) | <0.001 |
| Healthy Diet | 6,410 | (61%) | 808 | (11%) | 33 | (1%) | <0.001 |
| Genetic Risk Category | | | | | | | |
| Low Genetic Risk | 2,103 | (20%) | 1,509 | (20%) | 668 | (20%) | |
| Intermediate Genetic Risk | 6,319 | (60%) | 4,414 | (60%) | 1,983 | (60%) | 0.95 |
| High Genetic Risk | 2,094 | (20%) | 1,462 | (20%) | 670 | (20%) | |

TABLE 25
Baseline characteristics by lifestyle risk category, MDCS. Values represent
N (% with recorded values), mean (SD), or median (IQR). P-values computed via ANOVA for
continuous variables (TG modeled using Kruskal-Wallis test) and chi-square test for categorical
variables. FH (family history); CAD (coronary artery disease). Family history of premature
coronary artery disease refers to self-reported parental history of myocardial infarction.

| | Favorable | | Intermediate | | Unfavorable | | |
|---|---|---|---|---|---|---|---|
| | Lifestyle | | Lifestyle | | Lifestyle | | |
| | N = 7,210 | | N = 10,234 | | N = 4,945 | | P-value |
| Age, years | 58.2 | (7.7) | 58.1 | (7.8) | 57.4 | (7.5) | <0.0001 |
| Male Gender | 3,065 | (43%) | 3,722 | (36%) | 1,728 | (35%) | <0.0001 |
| History of Hypertension | 4,212 | (58%) | 6,149 | (60%) | 3,192 | (65%) | <0.0001 |
| History of Diabetes Mellitus | 279 | (4%) | 371 | (4%) | 254 | (5%) | <0.0001 |
| FH of CAD | 2,322 | (32%) | 3,350 | (33%) | 1,553 | (31%) | 0.26 |
| Body-mass Index, kg/m² | 24.9 | (2.9) | 25.4 | (3.6) | 27.4 | (5.2) | <0.0001 |
| Lipid Levels | | | | | | | |
| LDL Cholesterol, mg/dl | 160 | (38) | 161 | (38) | 164 | (40) | 0.06 |
| HDL Cholesterol, mg/dl | 55 | (15) | 54 | (15) | 50 | (13) | <0.0001 |
| Triglycerides, mg/dl | 97 | (72-134) | 102 | (76-141) | 117 | (86-162) | 0.0001 |
| Lipid-lowering Medication | 147 | (2.0%) | 227 | (2.2%) | 114 | (2.3%) | 0.58 |
| C-Reactive Protein, mg/L | 1.1 | (0.6-2.2) | 1.3 | (0.6-2.7) | 2.0 | (0.9-4.2) | 0.0001 |
| Healthy Lifestyle Factors | | | | | | | |
| No Current Smoking | 6,981 | (97%) | 7,924 | (77%) | 1,257 | (25%) | <0.0001 |
| Nonobese | 7,094 | (98%) | 9,316 | (91%) | 3,097 | (63%) | <0.0001 |
| Regular Physical Activity | 6,146 | (85%) | 2,747 | (27%) | 200 | (4%) | <0.0001 |
| Healthy Diet | 2,279 | (32%) | 481 | (5%) | 35 | (1%) | <0.0001 |
| Genetic Risk Category | | | | | | | |
| Low Genetic Risk | 1,444 | (20%) | 2,060 | (20%) | 974 | (20%) | |
| Intermediate Genetic Risk | 4,336 | (60%) | 6,145 | (60%) | 2,953 | (60%) | 0.82 |
| High Genetic Risk | 1,430 | (20%) | 2,029 | (20%) | 1,018 | (21%) | |

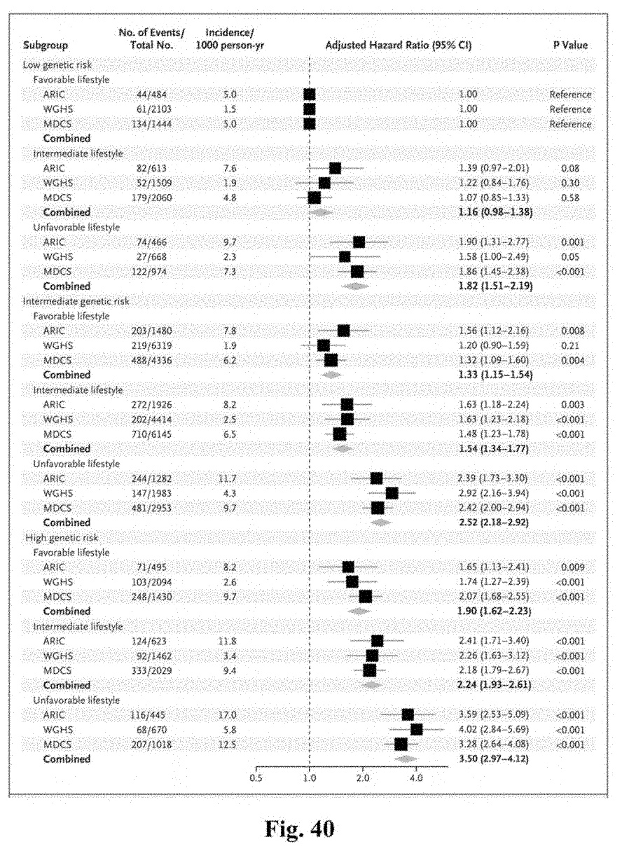

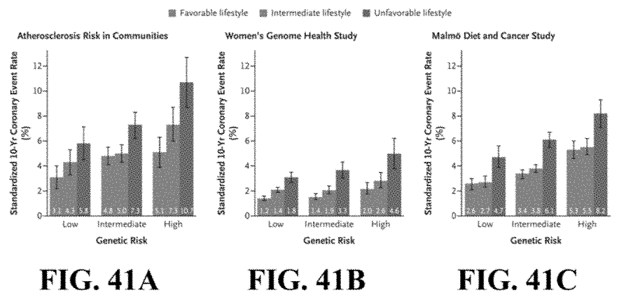

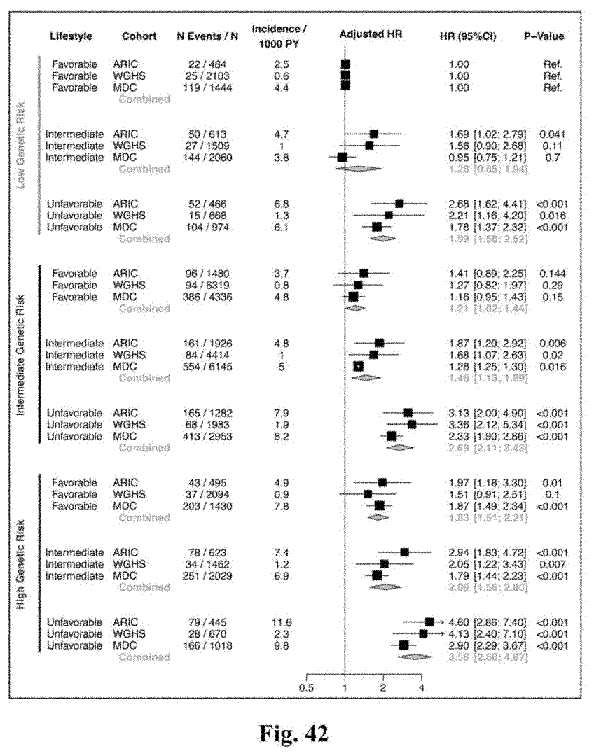

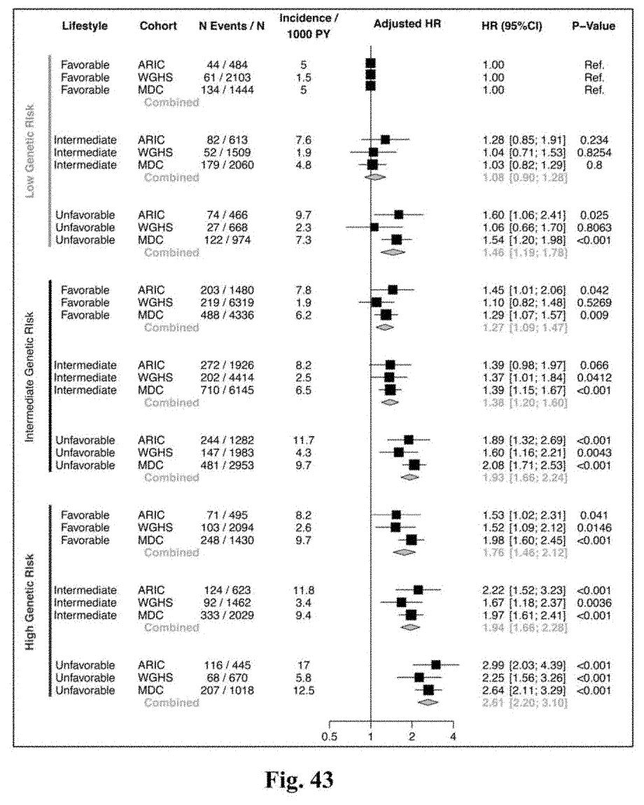

Within each category of genetic risk, lifestyle factors were strong predictors of coronary events (FIG. 40) Risk of Coronary Events, According to Genetic and Lifestyle Risk in the Prospective Cohorts). Adherence to a favorable lifestyle, as compared with an unfavorable lifestyle, was associated with a 45% lower relative risk among participants at low genetic risk, a 47% lower relative risk among those at intermediate genetic risk, and a 46% lower relative risk (hazard ratio, 0.54; 95% CI, 0.47 to 0.63) among those at high genetic risk. Among participants at high genetic risk, the standardized 10-year coronary event rates were 10.7% among those with an unfavorable lifestyle and 5.1% among those with a favorable lifestyle in the ARIC cohort, 4.6% and 2.0%, respectively, in the WGHS cohort, and 8.2% and 5.3% in the MDCS cohort (FIG. 41) 10-Year Coronary Event Rates, According to Lifestyle and Genetic Risk in the Prospective Cohorts.). Similarly, a low genetic risk was largely offset by an unfavorable lifestyle. Among participants at low genetic risk, standardized 10-year coronary event rates were 5.8% among those with an unfavorable lifestyle and 3.1% among those with a favorable lifestyle in the ARIC cohort, 1.8% and 1.2%, respectively, in the WGHS cohort, and 4.7% and 2.6% in the MDCS cohort. Similar patterns were noted after the exclusion of coronary revascularization from the composite end point (FIG. 42). Adjustment for traditional risk factors attenuated estimates, although the decreased risk among participants with a favorable lifestyle within each genetic risk category remained apparent (Table S15 and FIG. 43).

TABLE 26
Risk of coronary events according to genetic and lifestyle categories adjusted for
traditional risk factors. Cox regression models were adjusted for age, gender (in
ARIC and MDCS), randomization to VitaminE or aspirin (in WGHS), education level, and
principal components of ancestry (in ARIC and WGHS), presence of diabetes mellitus,
hypertension, family history of coronary artery disease, LDL cholesterol levels
(apolipoprotein B in MDCS), and HDL cholesterol levels (apolipoprotein A-I in MDCS).
Cohort-specific findings were combined using random effects meta-analysis.
Values displayed represent hazard ratios and 95% confidence intervals.

| | Atherosclerosis | Women's | | |
|---|---|---|---|---|
| | Risk in | Genome | Malmö Diet and | |
| | Communities | Health Study | Cancer Study | Combined |
| Genetic Risk Category | | | | |
| Low Risk | Reference | Reference | Reference | Reference |
| Intermediate Risk | 1.19 (1.00-1.41) | 1.25 (1.03-1.53) | 1.33 (1.20-1.48) | 1.28 (1.18-1.39) |
| High Risk | 1.70 (1.40-2.06) | 1.67 (1.35-2.08) | 1.88 (1.67-2.11) | 1.80 (1.64-1.97) |
| P-Trend | 3.4 × 10⁻⁸ | 1.6 × 10⁻⁶ | 6.4 × 10⁻²⁷ | |
| Lifestyle Risk Category | | | | |
| Favorable | Reference | Reference | Reference | Reference |
| Intermediate | 1.10 (0.94-1.28) | 1.17 (0.99-1.37) | 1.04 (0.96-1.14) | 1.08 (1.01-1.15) |
| Unfavorable | 1.46 (1.24-1.72) | 1.40 (1.17-1.69) | 1.52 (1.38-1.68) | 1.49 (1.38-1.61) |
| P-Trend | 4.1 × 10⁻⁶ | 0.0004 | 4.9 × 10⁻¹⁵ | |

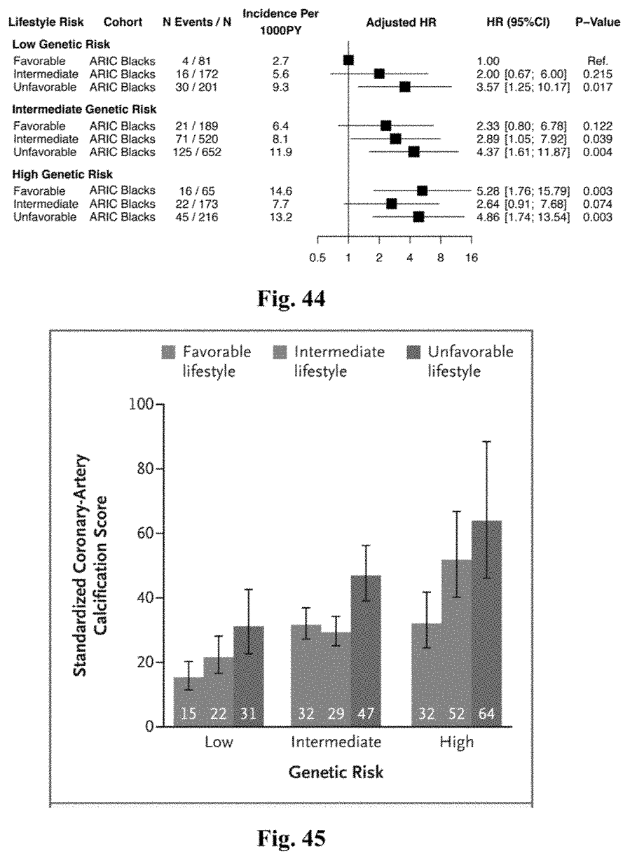

Despite a paucity of well-validated genetic loci in black populations, Applicant observed similar findings among black participants and white participants in the ARIC cohort (FIG. 44). However, additional data are needed to confirm the consistency of the effect in populations of African ancestry.

A cross-sectional analysis of 4260 of 4301 white participants with available data from the BioImage Study showed that both genetic and lifestyle factors were associated with coronary-artery calcification (stratified according to the baseline characteristics in Tables S16 and S17). The standardized calcification score was 46 Agatston units (95% CI, 39 to 54) among participants at high genetic risk, as compared with 21 Agatston units (95% CI, 18 to 25) among those at low genetic risk (P<0.001). The calcification score was similarly higher among participants with an unfavorable lifestyle than among those with a favorable lifestyle: 46 Agatston units (95% CI, 40 to 53) versus 28 Agatston units (95% CI, 25 to 31) (P<0.001). Within each subgroup of genetic risk, a significant trend was observed toward decreased coronary-artery calcification among participants who were more adherent to a healthy lifestyle (FIG. 45) Coronary-Artery Calcification Score in the BioImage Study, According to Lifestyle and Genetic Risk.).

TABLE 27
Baseline characteristics by genetic risk category, BioImage study. Values
represent N (% with recorded values), mean (SD), or median (IQR). P-values computed via
ANOVA for continuous variables (TG modeled using Kruskal-Wallis test) and chi-square test
for categorical variables. FH (family history); CAD (coronary artery disease). Family history
of premature coronary artery disease refers to self-reported parental history of myocardial
infarction.

| | | | Intermediate | | | | |
|---|---|---|---|---|---|---|---|
| | Low Risk | | Risk | | High Risk | | |
| | N = 846 | | N = 2,557 | | N = 857 | | P-value |
| Age, years | 68.9 | (6.1) | 69.1 | (6.1) | 69.1 | (5.7) | 0.69 |
| Male Gender | 405 | (48%) | 1,132 | (44%) | 341 | (40%) | 0.003 |
| History of Hypertension | 507 | (60%) | 1,553 | (61%) | 516 | (60%) | 0.90 |
| History of Diabetes Mellitus | 101 | (12%) | 329 | (13%) | 92 | (11%) | 0.25 |
| Family History of CAD | 312 | (37%) | 1,037 | (41%) | 368 | (43%) | 0.11 |
| Body-mass Index, kg/m² | 29.0 | (5.4) | 28.9 | (5.5) | 28.3 | (5.2) | 0.02 |
| Lipid Levels | | | | | | | |
| LDL Cholesterol, mg/dl | 111 | (33) | 114 | (33) | 114 | (32) | 0.08 |
| HDL Cholesterol, mg/dl | 57 | (16) | 56 | (16) | 56 | (15) | 0.29 |
| Triglycerides, mddl | 145 | (105-210) | 150 | (108-211) | 145 | (104-204) | 0.19 |
| Lipid-lowering Medication | 264 | (31%) | 893 | (35%) | 310 | (36%) | 0.07 |
| Healthy Lifestyle Factors | | | | | | | |
| No Current Smoking | 767 | (91%) | 2,333 | (91%) | 787 | (92%) | 0.69 |
| Non-obese | 518 | (61%) | 1,629 | (64%) | 582 | (68%) | 0.01 |
| Regular Physical Activity | 406 | (48%) | 1488 | (47%) | 373 | (44%) | 0.16 |
| Healthy Diet | 109 | (13%) | 377 | (15%) | 124 | (15%) | 0.40 |
| Lifestyle Risk Category | | | | | | | |
| 3-4 Healthy Lifestyle Factors | 293 | (35%) | 955 | (37%) | 316 | (37%) | |
| 2 Healthy Lifestyle Factors | 329 | (39%) | 932 | (36%) | 337 | (39%) | 0.30 |
| 0-1 Healthy Lifestyle Factors | 224 | (27%) | 670 | (26%) | 204 | (34%) | |

TABLE 28
Baseline characteristics by lifestyle risk category, BioImage study. Values
represent N (% with recorded values), mean (SD), or median (IQR). P-values computed via
ANOVA for continuous variables (TG modeled using Kruskal-Wallis test) and chi-square test
for categorical variables. FH (family history); CAD (coronary artery disease). Family history
of premature coronary artery disease refers to self-reported parental history of myocardial
infarction.

| | Favorable | | Intermediate | | Unfavorable | | |
|---|---|---|---|---|---|---|---|
| | Lifestyle | | Lifestyle | | Lifestyle | | |
| | N = 1,564 | | N = 1,598 | | N = 1,098 | | P-value |
| Age, years | 69.7 | (5.9) | 69.2 | (6.1) | 68.0 | (5.9) | <0.001 |
| Male Gender | 683 | (44%) | 687 | (43%) | 507 | (46%) | 0.17 |
| History of Hypertension | 870 | (56%) | 976 | (61%) | 730 | (67%) | <0.001 |
| History of Diabetes Mellitus | 107 | (7%) | 190 | (12%) | 225 | (21%) | <0.001 |
| Family History of CAD | 608 | (39%) | 652 | (41%) | 457 | (41%) | 0.11 |
| Body-mass Index, kg/m² | 26.0 | (3.3) | 28.5 | (5.1) | 33.2 | (5.6) | <0.001 |
| Lipid Levels | | | | | | | |
| LDL cholesterol, mg/dl | 115 | (31) | 114 | (33) | 110 | (34%) | <0.001 |
| HDL cholesterol, mg/dl | 60 | (16) | 56 | (15) | 51 | (14) | <0.001 |
| Triglycerides, mg/dl | 133 | (98-187) | 149 | (108-208) | 173 | (123-238) | <0.001 |
| Lipid-lowering Medication | 467 | (30%) | 550 | (34%) | 450 | (41%) | <0.001 |
| Healthy Lifestyle Factors | | | | | | | |
| No Current Smoking | 1,558 | (99.6%) | 1,497 | (94%) | 832 | (76%) | <0.001 |
| Non-obese | 1477 | (94%) | 1,080 | (68%) | 172 | (16%) | <0.001 |
| Regular Physical Activity | 1,423 | (91%) | 523 | (33%) | 21 | (2%) | <0.001 |
| Healthy Diet | 511 | (33%) | 96 | (6%) | 3 | (0.3%) | <0.001 |
| Genetic Risk Category | | | | | | | |
| Low Genetic Risk | 293 | (19%) | 329 | (21%) | 224 | (20%) | |
| Intermediate Genetic Risk | 955 | (61%) | 932 | (58%) | 670 | (61%) | 0.30 |
| High Genetic Risk | 316 | (20%) | 337 | (21%) | 204 | (19%) | |

### Discussion

In this study, Applicant have provided quantitative data about the interplay between genetic and lifestyle risk factors for coronary artery disease in three prospective cohorts and one cross-sectional study. High genetic risk was independent of healthy lifestyle behaviors and was associated with an increased risk (hazard ratio, 1.91) of coronary events and a substantially increased burden of coronary-artery calcification. However, within any genetic risk category, adherence to a healthy lifestyle was associated with a significantly decreased risk of both clinical coronary events and subclinical burden of coronary artery disease.

The results of this analysis support three noteworthy conclusions. First, our data indicate that inherited DNA variation and lifestyle factors contribute independently to a susceptibility to coronary artery disease. Our finding that a polygenic risk score has robust associations with incident coronary events is well aligned with previous studies of both primary and secondary prevention populations. www.nejm.org/doi/full/10.1056/NEJMoa1605086—ref16 (Kathiresan S, Melander O, Anevski D, et al. Polymorphisms associated with cholesterol and risk of cardiovascular events. N Engl J Med 2008; 358:1240-1249; Ripatti S, Tikkanen E, Orho-Melander M, et al. A multilocus genetic risk score for coronary heart disease: case-control and prospective cohort analyses. Lancet 2010; 376:1393-1400; Paynter N P, Chasman D I, Pare G, et al. Association between a literature-based genetic risk score and cardiovascular events in women. JAMA 2010; 303:631-637; Thanassoulis G, Peloso G M, Pencina M J, et al. A genetic risk score is associated with incident cardiovascular disease and coronary artery calcium: the Framingham Heart Study. Cire Cardiovasc Genet 2012; 5:113-121; Brautbar A, Pompeii L A, Dehghan A, et al. A genetic risk score based on direct associations with coronary heart disease improves coronary heart disease risk prediction in the Atherosclerosis Risk in Communities (ARIC), but not in the Rotterdam and Framingham Offspring, Studies. Atherosclerosis 2012; 223:421-426; Ganna A, Magnusson P K, Pedersen N L, et al. Multilocus genetic risk scores for coronary heart disease prediction. Arterioscler Thromb Vasc Biol 2013; 33:2267-2272; Mega J L, Stitziel N O, Smith J G, et al. Genetic risk, coronary heart disease events, and the clinical benefit of statin therapy: an analysis of primary and secondary prevention trials. Lancet 2015; 385:2264-2271; Tada H, Melander O, Louie J Z, et al. Risk prediction by genetic risk scores for coronary heart disease is independent of self-reported family history. Eur Heart J 2016; 37:561-567; Abraham G, Havulinna A S, Bhalala O G, et al. Genomic prediction of coronary heart disease. Eur Heart J 2016 Nov. 14; 37(43):3267-3278). Such findings support long-standing beliefs that genetic variants that are identifiable from birth alter coronary risk. (Müller C. Xanthomata, hypercholesterolemia, angina pectoris. Acta Med Scand 1938; 89:75-84; Gertier M M, Garn S M, White P D. Young candidates for coronary heart disease. J Am Med Assoc 1951; 147:621-625; Slack J, Evans K A. The increased risk of death from ischaemic heart disease in first degree relatives of 121 men and 96 women with ischaemic heart disease. J Med Genet 1966; 3:239-257). Aside from slight differences in LDL cholesterol levels and a family history of coronary artery disease, genetic risk was independent of traditionally measured risk factors.

Second, a healthy lifestyle was associated with similar relative risk reductions in event rates across each stratum of genetic risk. Although the absolute risk reduction that was associated with adherence to a healthy lifestyle was greatest in the group at high genetic risk, our results support public health efforts that emphasize a healthy lifestyle for everyone. An alternative approach is to target intensive lifestyle modification to those at high genetic risk, with the expectation that disclosure of genetic risk can motivate behavioral change. However, whether the provision of such information can improve cardiovascular outcomes remains to be determined.

Third, patients may equate DNA-based risk estimates with determinism, a perceived lack of control over the ability to improve outcomes. (White P D. Genes, the heart and destiny. N Engl J Med 1957; 256:965-969). However, our results provide evidence that lifestyle factors may powerfully modify risk regardless of the patient's genetic risk profile. Indeed, alternative analytic approaches that incorporate more stringent cutoffs or weight the relative effect for each healthy lifestyle factor may lead to an even more pronounced coronary risk gradient.

In conclusion, after quantifying both genetic and lifestyle risk among 55,685 participants in three prospective cohorts and one cross-sectional study, Applicant found that adherence to a healthy lifestyle was associated with a substantially reduced risk of coronary artery disease within each category of genetic risk.

### Example 3

Whole genome sequencing enables ascertainment of the complete spectrum of genetic variation—common and rare, coding and noncoding. Rapid declines in cost have led to substantial enthusiasm that such testing will further our understanding of complex trait genetics and permit DNA-based population stratification that could inform clinical management. (See Ashley E A., Towards precision medicine, Nat Rev Genet, 2016; 17(9):507-22). Here, Applicants test this hypothesis by performing high coverage whole genome sequencing in 2,369 individuals with myocardial infarction at an early age and compare their genome sequences with 4,218 coronary disease-free participants. Applicants determine the association of common single variants as well as rare variants in both coding and noncoding regions with disease risk and identify the prevalence and clinical impact of monogenic (single large-effect mutation) and polygenic (cumulative effect of many variants of small effect) risk pathways associated with myocardial infarction.

### Study Populations

The design of the VIRGO study has been previously described. (See Lichtman et al., Circ Cardiovasc Qual Outcomes, 2010; 3(6):684-93.) In brief, 3,501 participants hospitalized with an acute myocardial infarction, age 18 to 55 years, were enrolled between 2009 and 2012 from 103 United States and 24 Spanish hospitals using a 2:1 female-to-male enrollment design. Baseline patient data were collected by medical chart abstraction and standardized in-person patient interviews administered by trained personnel during the index acute myocardial infarction admission. Individuals with available DNA and who had provided written informed consent for genetic analysis were included in the present study.

The TAICHI cohort recruited Taiwanese Chinese individuals at four academic centers. (See Assimes et al., PLoS One, 2016; 11(3):e0138014). Individuals with coronary disease were identified as those with a history of myocardial infarction, coronary revascularization, or a stenosis of ≥50% in a major epicardial vessel demonstrated by angiography. All cases experienced an early-onset coronary event (men ≤50 years, women ≤60 years) in the context of normal circulating lipid levels (LDL cholesterol <130 mg/dl or total cholesterol <185 mg/dl). Controls were enrolled from an epidemiology study and from the several Hospital Endocrinology and Metabolism Departments either as outpatients or as their family members. Subjects with a history of CAD were excluded.

The design of the MESA study has been previously described and protocol available at www.mesa-nhlbi.org. (See Bild et al., Am J Epidemiol, 2002; 156:871-881). In brief, 6,181 men and women between the ages of 45 and 84 without prevalent cardiovascular disease were recruited between 2000-2002 from 6 United States communities. Individuals were excluded from the present study due if informed consent for genetic testing had not been obtained/was withdrawn, DNA was not available for sequencing, or incident cardiovascular disease (myocardial infarction, coronary revascularization, angina, peripheral arterial disease, stroke, resuscitated cardiac arrest, death due to cardiovascular causes) through the period of last available follow-up in December 2014. Fasting plasma triglyceride, total cholesterol, high density lipoprotein cholesterol (HDL-C) concentrations were measured as described previously. (See Tsai et al., Atherosclerosis, 2008; 200: 359-367). Low density lipoprotein-cholesterol (LDL-C) was calculated based on the Friedewald formula in participants with triglycerides <400 mg/dL. Lipoprotein(a) concentrations were available in 2,521 of 3,761 (67%) of sequenced individuals, measured via the a latex-enhanced turbidometric immunoassay (Denka Seiken, Tokyo, Japan) that is insensitive to Kringle 4 type 2 isoforms as reported previously. (See Guan et al., Arterioscler Thromb Vasc Biol, 2015 April; 35(4):996-1001).

Study participants with early-onset myocardial infarction were derived from the previously described Variation in Recovery: Role of Gender on Outcomes of Young AMI Patients (VIRGO) and TAICHI consortium and controls from the Multiethnic Study of Atherosclerosis (MESA) cohort and TAICHI consortium. The VIRGO study enrolled a multiethnic population of adult patients presenting to enrollment centers in the United States and Spain with a first myocardial infarction at age <55 years. (See Lichtman et al., Circ. Cardiovasc. Qual. Outcomes, 2010; 3(6):684-93). The TAICHI consortium enrolled patients with an early-onset coronary event (men ≤50 years, women ≤60 years) in the context of normal circulating lipid levels (LDL cholesterol <130 mg/dl or total cholesterol <185 mg/dl) and controls in academic centers in Taiwan. (See Assimes et al., PLoS One, 2016; 11(3):e0138014). The MESA study is a multiethnic prospective cohort that enrolled individuals in the United States free of cardiovascular disease between 2000 and 2002. (See Bild et al., Am. J. Epidemiol., 2002; 156:871-81). MESA participants were included as controls for this study if they remained free of incident cardiovascular disease through the end of 2014 (median follow-up 13.2 years).

TABLE 29
Baseline Demographics of Study Participants

| | Early-Onset MI | |
|---|---|---|
| | Cases | Controls |
| | N = 2369 | N = 4218 |
| Study | | |
| MESA | 0 | 3761 (89%) |
| VIRGO | 2081 (88%) | 0 |
| TAICHI | 288 (12%) | 457 (11%) |
| Race | | |
| White | 1537 (65%) | 1544 (37%) |
| Black | 336 (14%) | 962 (23%) |
| Asian | 328 (14%) | 961 (23%) |
| Hispanic | 168 (7%) | 751 (18%) |
| Male | 925 (39%) | 2019 (48%) |
| Age, years; Mean (SD) | 48 (6) | 61 (10) |
| Hypertension | 1415 (60%) | 1600 (38%) |
| Diabetes | 876 (37%) | 665 (16%) |
| Current Smoking | 1146 (49%) | 535 (13%) |
| Statin Use | 668 (29%) | 584 (14%) |
| Lipid Levels, Mean (SD) | | |
| LDL Cholesterol,* mg/dl | 122 (48) | 122 (35) |
| HDL Cholesterol, mg/dl | 41 (13) | 51 (15) |
| Triglycerides, mg/dl | 182 (205) | 132 (82) |
| Lipoprotein(a),^{†} mg/dl | N/A | 28 (31) |

* In order to estimate untreated levels of LDL cholesterol, values in those reporting statin use at time of ascertainment were divided by 0.7 as performed previously. (Khera et al.,J Am Coll Cardiol., 2016;67(22):2578-89; Dewey et al., N Engl J Med., 2016;374 (12):1123-1133; Stitziel et al., N Engl J Med., 2014;371(22):2072-2082).
^{†} Lipoprotein(a) concentrations available in 2,521 controls from the MESA cohort.

### Whole Genome Sequencing

Whole genome sequencing was performed using the Illumina HiSeqX platform at the Broad Institute of Harvard and MIT (Cambridge, Mass.). DNA samples were received into the Genomics Platform's Laboratory Information Management System via a scan of the tube barcodes using a Biosero flatbed scanner. This registers the samples and enables the linking of metadata based on well position. All samples are then weighed on a BioMicro Lab's XL20 to determine the volume of DNA present in sample tubes. Following this the samples are quantified in a process that uses PICO-green flourescent dye. Once volumes and concentrations are determined the samples are then handed off to the Sample Retrieval and Storage Team for storage in a −20° Celsius freezer.

Libraries were constructed and sequenced on the Illumina HiSeqX with the use of 151-bp paired-end reads for whole-genome sequencing. Output from Illumina software was processed by the Picard data-processing pipeline to yield BAM files containing well-calibrated, aligned reads. All sample information tracking was performed by automated LIMS messaging.

Samples undergo fragmentation by means of acoustic shearing using Covaris focused-ultrasonicator, targeting 385 bp fragments. Following fragmentation, additional size selection is performed using a SPRI cleanup. Library preparation is performed using a commercially available kit provided by KAPA Biosystems (product KK8202) and with palindromic forked adapters with unique 8 base index sequences embedded within the adapter (purchased from IDT). Following sample preparation, libraries were quantified using quantitative PCR (kit purchased from KAPA biosystems) with probes specific to the ends of the adapters. This assay was automated using Agilent's Bravo liquid handling platform. Based on qPCR quantification, libraries were normalized to 1.7 nM. Samples are then pooled into 24-plexes and the pools are once again qPCRed. Samples were then combined with HiSeq X Cluster Amp Mix 1, 2 and 3 into single wells on a strip tube using the Hamilton Starlet Liquid Handling system.

Cluster amplification of the templates was performed according to the manufacturer's protocol (Illumina) using the Illumina cBot. Flowcells were sequenced on Hi Seq X with sequencing software HiSeq Control Software (HCS) version 3.3.76, then analyzed using RTA2. The following versions were used for aggregation, and alignment to hg19_decoy reference: picard (latest version available at the time of the analysis), GATK (3.1-144-g00f68a3) and BwaMem (0.7.7-r441).

A sample was considered sequence complete when the mean coverage was ≥30× (for the MESA cohort) or ≥20× (for VIRGO and TAICHI cohorts). Two quality control metrics that are reviewed along with the coverage are the sample Fingerprint LOD score and % contamination. At aggregation, Applicants did an all-by-all comparison of the read group data and estimate the likelihood that each pair of read groups is from the same individual. If any pair had a LOD score <−20.00, the aggregation does not proceed and is investigated. FP LOD > or =3 is considered passing concordance with the sequence data (ideally Applicants see LOD >10). A sample will have an LOD of 0 when the sample failed to have a passing fingerprint. Fluidigm fingerprint is repeated once if failed. Read groups with fingerprints <−3.00 were blacklisted from the aggregation. Sample genotypes were determined via a joint callset using the Genome Analysis Toolkit Haplotype Caller.

Reads were aligned using to the human reference genome hg19.

Sample Quality Control.

6,809 individuals underwent whole genome sequencing, of whom 222 (3.3%) were excluded based on sequencing quality control metrics (Table 30). Sample exclusion criteria included:
1. DNA Contamination >5%
2. Mean coverage <20×
3. Sample duplicates/Identical Twins (as assessed by PI_HAT ≥0.95)
4. First or second degree relatives of another study participant (Kinship coefficient >0.0884)
5. Variant Call Rate <95%
6. Genotype/phenotype Sex Discordance or ambiguous sex (0.5<Fₛₜₐₜ<0.8)

TABLE 30
Sample Quality Control Criteria

| | Thresholds | MESA | VIRGO | TAICHI | Total |
|---|---|---|---|---|---|
| Initial Sample Size | | 3932 | 2101 | 776 | 6809 |
| Contamination | >5.0% | 19 | 3 | 0 | 22 |
| Raw Mean Coverage | <20X | 1 | 2 | 1 | 4 |
| Duplicates/Twins | PI-Hat ≥0.95 | 2 | 10 | 3 | 15 |
| 1^{st}/2^{nd} Degree | Kinship | | | | |
| Relatives | Coefficient | 148 | 2 | 2 | 152 |
| | >0.0884 | | | | |
| Post-QC Call Rate | <95% | 0 | 3 | 18 | 21 |
| Sex Check | 0.5 < Fstat < 0.8 | 1 | 0 | 7 | 8 |

| | | | | |
|---|---|---|---|---|
| Total Cases | 0 | 2081 | 288 | 2369 |
| Total Controls | 3761 | 0 | 457 | 4218 |

| | |
|---|---|
| Total Sample Size | 6587 |

Variant Quality Control.

After completion of sample level quality control, variant quality control was performed using the Hail software package (github.com/hail-is/hail). (Ganna et al., Nat Neurosci., 2016; 19(12):1563-1565). In total, 17.6 of 152.2 million (12%) of single nucleotide polymorphisms and 12.0 of 23.4 million (52%) of insertion-deletions variants were filtered from subsequent analysis (Table 30).

### Variant Exclusion Criteria Included:

1. Failure by the Genome Analysis Toolkit Variant Quality Score Recalibration metric, (McKenna et al., Genome Res., 2010; 20(9):1297-1303) a machine learning algorithm designed to balance sensitivity (calling genuine variants) and specificity (limit false positive variant calls)
2. Variants in low-complexity regions of the genome that preclude accurate read alignment as previously defined (Li H., Bioinformatics., 2014; 30(20):2843-51)
3. Variants in segmental duplications of the genome
4. Quality by depth score <2 (for single nucleotide polymorphisms) or <3 (for insertion-deletions)
5. Call rate <95%
6. Race specific Hardy-Weinberg dysequlibrium p-value <1×10⁻⁶ in control individuals.

TABLE 31
Variant Quality Control Criteria

| | | Single | |
|---|---|---|---|
| | | Nucleotide | Insertion/ |
| | All Variants | Polymorphisms | Deletions |
| Initial Variant Call File | 175,556,625 | 152,160,879 | 23,395,746 |
| Variant Quality Score | 9,084,291 | 7,964,813 | 1,119,478 |
| Recalibration | | | |
| Low-complexity Regions | 13,878,065 | 4,506,484 | 9,371,581 |
| Segmental Duplications | 2,605,056 | 2,298,904 | 306,152 |
| Call Rate <95% | 3,745,945 | 2,574,015 | 1,171,930 |
| Quality/Depth or Hardy | 345,720 | 269,578 | 76,142 |
| Weinberg p-value | | | |
| Final Variant Call File | 145,897,548 | 134,547,085 | 11,350,463 |

Race Subgroup Inference.

A panel of approximately 16,000 ancestry informative markers (Hoggart et al., Am J Hum Genet., 2003; 72(6):1492-1504) (AIMs) identified across six continental populations (Libiger O, Schork N J., Front Genet., 2012; 3:322) was chosen to derive principal components (PCs) of ancestry for all samples that passed quality control. Principal component analysis was performed using EIGENSTRAT. (See Price et al., Nat Genet., 2006; 38:904-909).

In order to assign a race to individuals without self-reported race or with discordant self-reported race and PC ancestry, a k-nearest neighbors (k-NN) classifier (Fix E, Hodges J L. Discriminatory analysis: Non-parametric discrimination: Consistency properties. Texas: USAF School of Aviation Medicine. 1951; pp 261-279; Cover T, Hart P., IEEE Trans Inf Theory, 1967; 13:21-27.) was applied using the first five PCs of ancestry. This analysis was done using the k-NN implementation from the Scikit-learn library in Python. (See Pedregosa et al., Journal of Machine Learning Research, 2011; 12:2825-2830). The classifier was built using MESA samples after removing 25 individuals with discordant self-reported race and PC ancestry as determined by visual inspection of PC1 and PC2. The remaining MESA samples were split into a training set (n=2490) and test set (n=1246). A k-NN (k=5) classifier was built using self-reported race as the dependent variable (1: White/Caucasian, 2: Chinese American, 3: Black/African-American, 4: Hispanic) and PC1 to PC5 as features. The classifier had a 98.1% reclassification rate in the test set, with misclassifications generally occurring for Hispanic individuals. This classifier was then applied to all 6,587 samples to generate inferred race. Inferred race and self-reported race were concordant in 6,383 of 6,576 (97%) of sample with nonmissing self-reported race.

### Genetic Association Testing

The relationship of common (allele frequency ≥0.01) biallelic individual single nucleotide polymorphisms or short insertion-deletion (<10 base pairs) variants with early-onset myocardial infarction was tested.

Single Variant Testing.

Single nucleotide polymorphisms and insertion-deletion variants with allele frequency ≥1% were tested for association with early-onset myocardial infarction using logistic regression with adjustment for the first four principal components of ancestry.

Coding Variant Gene Burden Testing.

The group of rare (allele frequency <1%) coding variants tested for each gene was composed of 1) loss-of function variants 2) missense variants predicted to be damaging by each 5 of 5 computer prediction algorithms 3) variants annotated to be pathogenic in the ClinVar online genetics database. Loss-of function variants were identified with LOFTEE (Loss-Of-Function Transcript Effect Estimator), a plugin for the Ensembl Variant Effect Predictor (VEP). (See McLaren et al., Genome Biol., 2016; 17(1):122; Lek et al., Nature, 2016; 536(7616):285-91). They were included when they were deemed as high confidence loss-of function. The LOFTEE assessment includes stop-gained, splice site disrupting and frameshift variants. Rare missense variants were included if they were annotated as damaging or possible damaging by each of 5 computer prediction algorithms (SIFT, PolyPhen2-HumDiv, Polyphen2-HumVar, LRT, MutationTaster) as previously performed. (See Purcell et al., Nature, 2014; 506:185-90; Khera et al., JAm Coll Cardiol., 2016; 67(22):2578-89; Khera et al., J Am Coll Cardiol., 2016; 67(22):2578-89). Pathogenic variants were identified with the February 2017 release of the ClinVar database [github.com/macarthur-lab/clinvar] using the ‘clinical significance’ annotation. (See Landrum et al., Nucleic Acids Res. 2014; 42(database issue):D980-D985). Variants were included if at least one entry was assigned a ‘pathogenic’ clinical significance and there were no conflicting interpretations (e.g. simultaneous annotation as ‘uncertain,’ ‘benign,’ or ‘protective’). Variants assigned as benign were excluded from subsequent analyses. A collapsed burden test was performed with EPACTS v3.2.6 (EPACTS: Efficient and Parallelizable Association Container Toolbox [Internet]. [cited 2017 Apr. 13]; Available from: genome.sph.umich.edu/wiki/EPACTS) using a logistic Wald test between the outcome and 0/1-collapsed variants, including the first four principal components of ancestry were as covariates. Genes were tested when at least two variants met the inclusion criteria and the cumulative allele frequency of the damaging variants was above 0.001.

Regulatory Variant Gene Burden Testing.

Rare (MAF<1%) regulatory non-coding variants for testing were identified based on their location within enhancers and promoters in aortic tissue. Enhancer and promoter regions were annotated based on the Roadmap Epigenomics project. (See Roadmap Epigenomics Consortium., Kundaje et al., Nature, 2015; 518(7539):317-30). These regions were defined based on a chromatin state model (imputed data, 25 states) using observed DNaseI data, (Reg2Map: HoneyBadger2-impute [Internet]. [cited 2017 Apr. 13]; Available from: personal.broadinstitute.org/meuleman/reg2map/HoneyBadger2-impute_release/) selecting DNaseI regions were with −log₁₀(p)≥10. The following states were included to define promoter regions: active TSS, promoter upstream TSS, promoter downstream TSS, promoter downstream TSS, poised promoter and bivalent promoter. The following states were included to define enhancer regions: transcribed 5′ preferential and enh, transcribed 3′ preferential and enh, transcribed and weak enhancer, active enhancer 1, active enhancer 2, active enhancer flank, weak enhancer 1, weak enhancer 2 and possible enhancer. For each tissue or cell line the variants in promoter or enhancer regions were grouped to a gene, based on their proximity to the TSS. The inclusion region for promoters was defined as TSS+/−5 kb or the end of the canonical transcript, if the canonical transcript was shorter than 5000 bases. The inclusion region for enhancers was defined as TSS+/−20 kb or the end of the canonical transcript, if the canonical transcript was shorter than 20000 bases. Variants that fell within the exon bounds+/−5 base pairs of the canonical transcript were excluded. A sequence kernel association test (SKAT-O) (Lee et al., Biostatistics., 2012 September; 13(4):762-75) was performed with EPACTS v3.2.6 for each regulatory non-coding gene group and tissue or cell line. The first four principal components of ancestry were included as covariates in the models. Genes were tested when at least two variants met the inclusion criteria and the cumulative allele frequency of the damaging variants was above 0.001.

Gene-based coding variant testing was performed by aggregating rare (minor allele frequency <0.01) variants that lead to loss-of-function, were annotated as ‘Pathogenic’ in the ClinVar clinical genetics database (see Landrum et al., Nucleic Acids Res., 2014 January; 42 (Database issue):D980-85), or missense variants classified as damaging or possibly damaging by each of five computer prediction algorithms. (See Khera et al., JAMA, 2017; 317(9):937-946; Do et al., Nature, 2015; 518(7537):102-6). Tissue-specific regulatory burden testing was performed by aggregating rare variants in promoter or enhancer regions and assigning them to genes based on chromosomal proximity to a gene's transcription start site (within 5 kilobases for promoters and 20 kilobases for enhancer regions). (See Roadmap Epigenomics Consortium, Kundaje et al., Nature, 2015; 518(7539):317-30). For both the coding and regulatory burden testing, genes were included in the analysis if the cumulative allele frequency in the study population was >0.001 and at least 2 variants were observed.

The association of the three established monogenic risk pathways for early-onset myocardial infarction included variants in LDLR, APOB, or PCSK9 linked with familial hypercholesterolemia, (See Do et al., Nature, 2015; 518(7537):102-6; Khera et al., J. Am. Coll. Cardiol., 2016; 67(22):2578-89). LPL or APOA5 associated with defective clearance of triglyceride rich lipoproteins, (see Do et al., Nature, 2015; 518(7537):102-6; Khera et al., JAMA, 2017; 317(9):937-946) or at least two risk variants associating with lipoprotein(a) as previously described. (See Clarke et al., N. Engl. J. Med., 2009; 361(26):2518-28).

### Polygenic Risk Score

A polygenic risk score (PRS) for CAD was built using a p-value and LD-driven clumping procedure in PLINK version 1.90b (--clump). (See Chang et al., GigaScience, 2015; 4). Input included summary CAD association statistics for 8.3 million SNPs from a large 1000 Genomes imputed GWAS of primarily European individuals (CARDIoGRAMplusC4D Consortium, A comprehensive 1000 Genomes-based genome-wide association meta-analysis of coronary artery disease. Nat Genet., 2015; 47:1121-1130) and a reference LD panel of 503 European samples from 1000 Genomes phase 3 version 1. (See The 1000 Genomes Project Consortium, A global reference for human genetic variation, Nature, 2015; 526(7571):68-74). In brief, the algorithm forms clumps around SNPs with association p-values less than a provided threshold. Each clump contains all SNPs within 250 kb of the index SNP that are also in LD with the index SNP as determined by a provided r2 threshold in the LD reference. The algorithm iteratively cycles through all index SNPs, beginning with the smallest p-value, only allowing each SNP to appear in one clump. The final output should contain the most significantly CAD associated SNP for each LD-based clump across the genome. A PRS was built containing the index SNPs of each clump with association estimate betas (log odds) as weights.

PRSs were created over a range of p-value (1, 0.5, 0.05, 5×10-4, 5×10-6, 5×10-8) and r2 (0.2, 0.4, 0.6, 0.8) thresholds. To determine the best score, Applicants applied each to an independent set of 4,831 European CAD cases and 115,455 European controls from the UK Biobank (Sudlow et al., PLoS Med., 2015; 12: e1001779) using PLINK 1.90b (Chang et al., GigaScience, 2015; 4) (--score). Scores were generated by multiplying the number of risk alleles for each variant by the respective weight, and then summing across all variants in the score. Missing values were imputed to the mean genotype of that variant estimated by inferred ancestry group.

Beginning in 2006, individuals aged 45 to 69 years old were recruited from across the United Kingdom for participation in the UK Biobank Study. (See Sudlow et al., PLoS Med., 2015; 12: e1001779). At enrollment, a trained healthcare provider ascertained participants' medical histories through verbal interview. In addition, participants' electronic health records (EHR) including inpatient International Classification of Disease (ICD-10) diagnosis codes and Office of Population and Censuses Surveys (OPCS-4) procedure codes, were integrated into UK Biobank. Individuals were defined as having CAD based on at least one of the following criteria:
1) Myocardial infarction (MI), coronary artery bypass grafting, or coronary artery angioplasty documented in medical history at time of enrollment by a trained nurse
2) Hospitalization for ICD-10 code for acute myocardial infarction (121.0, 121.1, 121.2, 121.4, 121.9)
3) Hospitalization for OPCS-4 coded procedure: coronary artery bypass grafting (K40.1-40.4, K41.1-41.4, K45.1-45.5)
4) Hospitalization for OPCS-4 coded procedure: coronary angioplasty with or without stenting (K49.1-49.2, K49.8-49.9, K50.2, K75.1-75.4, K75.8-75.9)
   Other Individuals were Defined as Controls.

A polygenic risk score provides a quantitative assessment of the cumulative risk associated with multiple common risk alleles for each individual. Scores for each individual participant are created by adding up the number of risk alleles at each variant and then multiplying the sum by the literature-based effect size. (See Tada et al., Eur Heart 1, 2016; 37(6):561-7; Khera et al., N Engl J Med., 2016; 375(24):2349-2358; Abraham et al., Eur Heart J, 2016; 37(43):3267-3278). Applicants previously demonstrated that a literature-based polygenic risk score comprised of 50 genetic variants that have exceeded genome-wide levels of significance is associated with incident coronary events. (See Tada et al., Eur Heart 1, 2016; 37(6):561-7; Khera et al., N. Engl. J. Med., 2016; 375(24):2349-2358). However, the inclusion of additional subthreshold variants in a polygenic risk score may confer additional predictive value. (See Abraham et al., Eur Heart J., 2016; 37(43):3267-3278). In order to test this hypothesis, Applicants derived 24 distinct polygenic risk scores using summary statistics for 8.3 million single nucleotide polymorphisms of a previously reported GWAS study and an independent reference panel of whole genome sequence data from 503 European individuals. (See The 1000 Genomes Project Consortium, A global reference for human genetic variation, Nature, 2015; 526(7571):68-74; Nikpay et al., Nat. Genet., 2015; 47(10):1121-30). These 24 scores varied with regard to inclusions thresholds for previously reported p-value for association with coronary disease and degree of independence from other variants in the score. In order to determine which of these scores had the best predictive capacity, an independent validation dataset from the UK Biobank was assembled. (See Sudlow et al., PLoS Med., 2015; 12:e1001779). Each of these 24 scores was tested for association with coronary artery disease in UK Biobank and the score with the highest area under the curve was selected. This score was then applied to the whole genome sequencing dataset in order to determine the association of this polygenic risk score with myocardial infarction.

### Statistical Analysis

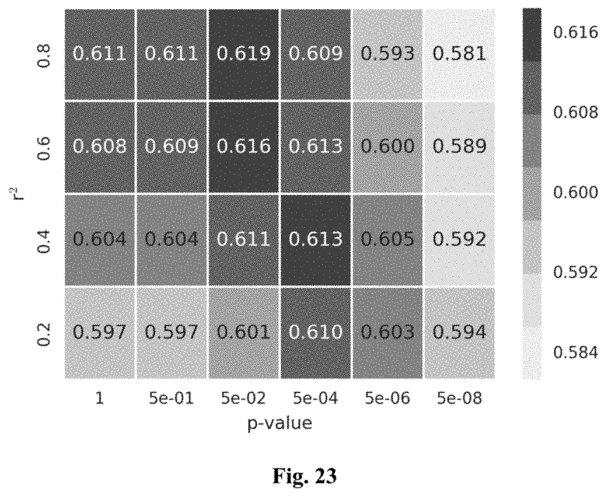

The association between each PRS with CAD status was determined using logistic regression adjusted for the first four principal components of ancestry. Area under the curve (AUC) was used to determine model discrimination. While each PRS showed a highly significant association with CAD status, the best PRS consisted of 116,859 SNPs and had an AUC of 0.619 (FIG. 23, Table 32). To account for potential strand flips, Applicants removed all C/G and A/T SNPs from the 116,859 SNP score and recalculated the PRS in the UK Biobank using the remaining 99,513 SNPs. This reduced score was strongly correlated with the full score (r²=0.99) and showed similar discrimination (AUC=0.618).

TABLE 32
Polygenic Risk Scores Evaluated in Testing Dataset from the UK Biobank

| | | | | | OR Top | |
|---|---|---|---|---|---|---|
| | | | # SNPs (%) | | vs. | OR Per |
| | | | in UKBB | | Bottom | SD |
| r² | p-value | # SNPs | (INFO >.3) | AUC | Quintile | Increment |
| 0.2 | 1 | 685,059 | 679,899 (99.3%) | 0.5967 | 2.65 | 1.42 |
| 0.2 | 5e−1 | 447,583 | 445,056 (99.4%) | 0.5972 | 2.62 | 1.42 |
| 0.2 | 5e−2 | 61,974 | 61,754 (99.6%) | 0.6012 | 2.64 | 1.45 |
| 0.2 | 5e−4 | 1,354 | 1,351 (99.8%) | 0.6100 | 3.18 | 1.48 |
| 0.2 | 5e−6 | 201 | 201 (100%) | 0.6034 | 2.72 | 1.44 |
| 0.2 | 5e−8 | 78 | 78 (100%) | 0.5938 | 2.59 | 1.38 |
| 0.4 | 1 | 1,057,321 | 1,052,079 (99.5%) | 0.6038 | 2.77 | 1.46 |
| 0.4 | 5e−1 | 643,673 | 641,107 (99.6%) | 0.6035 | 2.81 | 1.46 |
| 0.4 | 5e−2 | 77,045 | 76,823 (99.7%) | 0.6110 | 2.97 | 1.50 |
| 0.4 | 5e−4 | 1,695 | 1,692 (99.8%) | 0.6134 | 3.24 | 1.50 |
| 0.4 | 5e−6 | 268 | 268 (100%) | 0.6052 | 2.71 | 1.45 |
| 0.4 | 5e−8 | 106 | 106 (100%) | 0.5918 | 2.53 | 1.38 |
| 0.6 | 1 | 1,477,171 | 1,471,859 (99.6%) | 0.6085 | 2.96 | 1.48 |
| 0.6 | 5e−1 | 843,539 | 840,939 (99.7%) | 0.6086 | 2.95 | 1.49 |
| 0.6 | 5e−2 | 93,300 | 93,076 (99.8%) | 0.6160 | 3.10 | 1.53 |
| 0.6 | 5e−4 | 2,143 | 2,140 (99.9%) | 0.6128 | 3.13 | 1.50 |
| 0.6 | 5e−6 | 371 | 371 (100%) | 0.5996 | 2.67 | 1.43 |
| 0.6 | 5e−8 | 150 | 150 (100%) | 0.5888 | 2.42 | 1.38 |
| 0.8 | 1 | 2,043,188 | 2,037,808 (99.7%) | 0.6109 | 3.00 | 1.49 |
| 0.8 | 5e−1 | 1,103,850 | 1,101,216 (99.8%) | 0.6112 | 2.99 | 1.50 |
| 0.8 | 5e−2 | 116,859 | 116,632 (99.8%) | 0.6185 | 3.28 | 1.54 |
| 0.8 | 5e−4 | 2,919 | 2,916 (99.9%) | 0.6088 | 3.09 | 1.48 |
| 0.8 | 5e−6 | 541 | 541 (100%) | 0.5929 | 2.52 | 1.39 |
| 0.8 | 5e−8 | 218 | 218 (100%) | 0.5814 | 2.26 | 1.34 |

| | | | | | |
|---|---|---|---|---|---|
| Tada et al³¹ | 50 | 50 (100%) | 0.5841 | 2.21 | 1.34 |
| Abraham et al³² | 49,310 | 49,160 (99.7%) | 0.5906 | 2.49 | 1.38 |

The association of genetic variants with early-onset myocardial infarction, tested either individually or via burden testing, was tested using logistic regression, adjusted for four principal components of ancestry. Race-specific quintiles of the polygenic risk score were derived and risk estimates compared to previously published scores. (See Tada et al., Eur. Heart J., 2016; 37(6):561-7; Khera et al., N. Engl. J. Med., 2016; 375(24):2349-2358; Abraham et al., Eur. Heart J., 2016; 37(43):3267-3278). The relationship of monogenic risk pathway variants with intermediate phenotypes of circulating lipid values was determined using linear regression, adjusting for age, sex, cohort, and four principal components of ancestry.

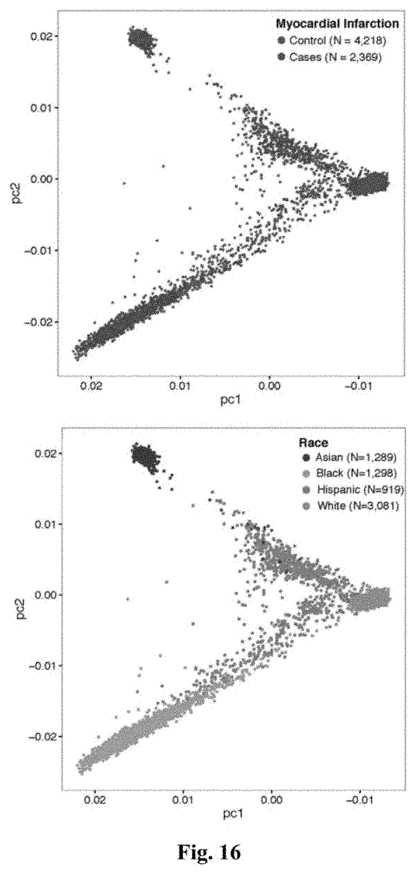

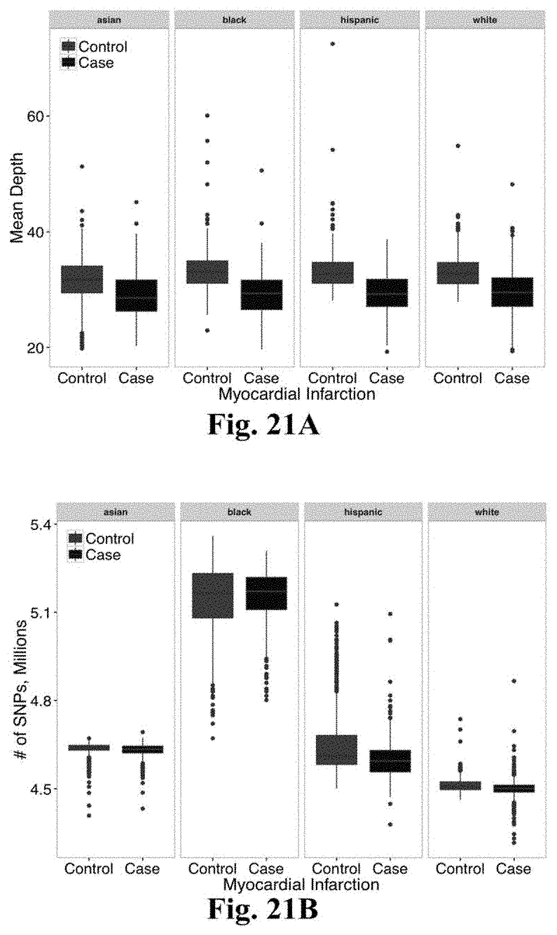

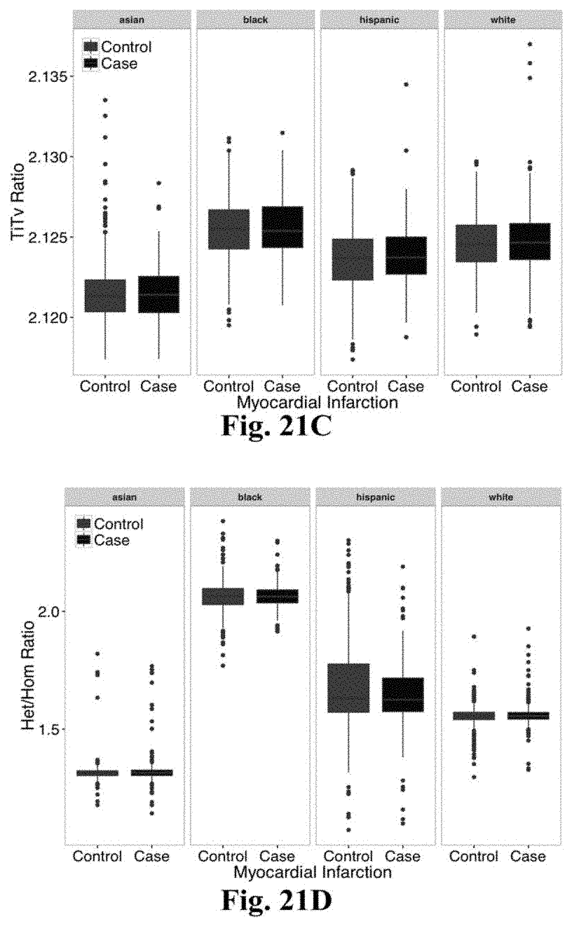

High-coverage whole genome sequencing was performed on 6,809 individuals. 222 (3.3%) of the original samples were excluded based on sequencing quality control metrics or relatedness, resulting in a final study population of 6,587 individuals—2,369 cases and 4,218 controls. This multiethnic population included 3,081 (47%) white, 1,298 black (20%), 1,289 Asian (20%) and 919 (14%) Hispanic participants Tables 11 & 12). Principal components analysis demonstrated that cases and controls were well-matched according to genetic ancestry (FIG. 16). Mean sequencing depth was 31.7× (SD 3.8) across the study cohorts with similar quality metrics observed across cases and controls (FIG. 21).

145,897,548 genetic variants were observed in sequenced individuals, of which the majority were in either intronic (50.6%) or intergenic (32.8%) regions of the genome (Table 14 & FIG. 17). 1,733,298 (1.2%) of variants were in the protein-coding region of the genome, of which the majority (55%) were missense variants leading to a single change in amino acid sequence. Furthermore, the majority of observed variants were rare in the population—55% were singletons (observed only once among sequenced individuals) and an additional 23% were observed in fewer than 7 of the 6,587 sequenced individuals (<1:1,000).

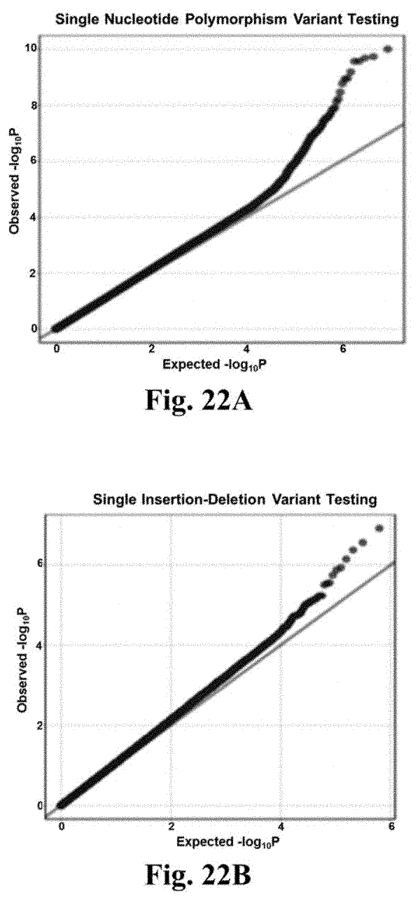

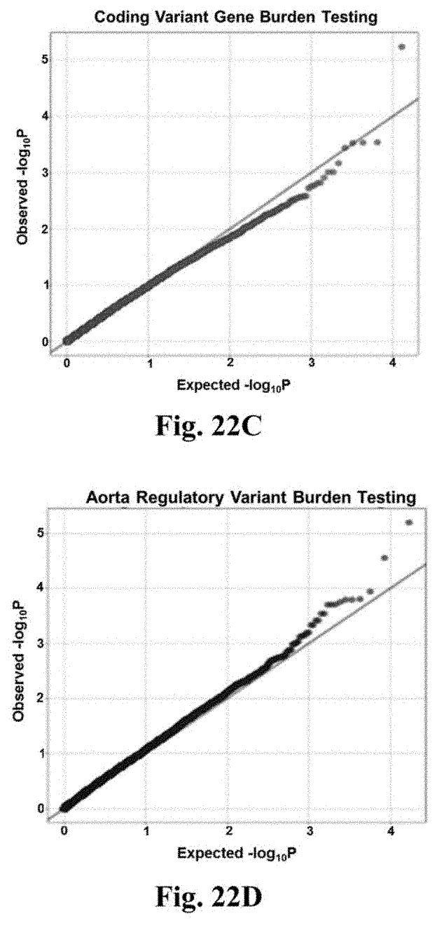

Single variant testing of 9,655,540 single nucleotide polymorphisms with allele frequency ≥1% was performed (genomic inflation factor [λ]=1.077), replicating two known associations at the recommended (see Pulit et al., The multiple testing burden in sequencing-based disease studies of global populations, bioRxiv 053264; doi: doi.org/10.1101/053264) genome-wide level of significance for sequencing studies of P<5×10-9 (FIG. 22). rs3798220, an intronic variant in the LPA gene (allele frequency=0.05) was associated with increased risk of myocardial infarction (odds ratio 1.77, P=9×10-11). Similarly, rs1333049, a common variant at the 9p21 locus (allele frequency=0.45) was associated with increased risk (odds ratio 1.29; p=1.8×10-10). 246 variants with suggestive evidence of association (P<1×10-5) were noted. Subsequent analysis of 621,476 insertion-deletion variants did not reveal statistically significant associations (genomic inflation factor [λ]=1.085), although 21 variants with suggestive evidence of association (P<1×10⁻⁵) were noted.

Applicants tested for an excess burden among cases of rare (allele frequency <1%) damaging coding variants across 12,989 genes. Consistent with previous results derived from exome sequencing, see Do et al., Exome sequencing identifies rare LDLR and APOA5 alleles conferring risk for myocardial infarction, Nature, 2015; 518(7537):102-6, the top signal was for damaging variants in LDLR, conferring an odds ratio of 3.47 (95% CI 2.02-5.95; p=5.8×10-6). Applicants also combined rare non-coding variants in aortic tissue-specific enhancer and promoter regions based on proximity to protein-coding genes, although no statistically significant associations were identified. For both coding and noncoding gene burden testing, genes with suggestive evidence of association (P<0.05) are provided (FIG. 22). Similarly null results were obtained when enhancer and promoter regions were annotated based on endothelial cell, liver, or monocyte tissues.

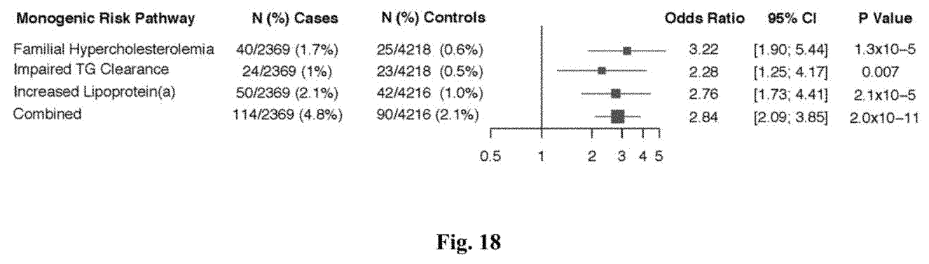

A mutation in a monogenic risk pathway for myocardial infarction was observed in 4.8% of sequenced individuals (FIG. 18). Mutations linked to familial hypercholesterolemia were identified in 1.7% of those with early-onset myocardial infarction and associated with a 53 mg/dl (95% CI 43-63) increase in circulating LDL cholesterol and odds ratio (OR) of 3.2 (95% CI 1.9-5.4) for myocardial infarction. This effect was most pronounced among heterozygous carriers of a fully inactivating mutation in LDLR (as compared to variants annotated as pathogenic in ClinVar or rare missense variants in LDLR predicted to be damaging), identified in 7 (0.3%) of myocardial infarction cases and 0 controls. These mutations were associated with a 176 mg/dl (95% CI 142-210) increase in circulating LDL cholesterol (Table 33).

TABLE 33
Association of Familial Hypercholesterolemia Mutations with LDL
Cholesterol and Risk of Myocardial Infarction

| | | | Impact on | |
|---|---|---|---|---|
| | | | LDL | |
| | N (%) of | N (%) of | Cholesterol, | Odds Ratio |
| Variant | 4,218 | 2,369 | mg/dl | for MI |
| Classification | Controls | MI Cases | (95% CI) | (95% CI) |
| Loss of Function, | 0 (0%) | 7 (0.3%) | +176 | N/A |
| LDLR | | | (142-210) | |
| | | | P < 0.001 | |
| Clinvar ‘Pathogenic’ | 7 (0.2%) | 13 (0.5%) | +49 | 3.60 |
| | | | (31-67) | (1.41-9.89) |
| | | | P < 0.001 | P = 0.009 |
| Predicted Damaging | 16 (0.4%) | 20 (0.8%) | +37 | 2.48 |
| Missense | | | (24-50) | (1.25-5.00) |
| | | | P < 0.001 | P = 0.01 |
| Combined | 25 (0.6%) | 40 (1.7%) | +53 | 3.22 |
| | | | (43-53) | (1.92-5.50) |
| | | | P < 0.001 | P < 0.001 |

Variants associated with defects in triglyceride lipolysis were noted in 24 (1.0%) of myocardial infarction cases and associated with 54 mg/dl (95% CI 15-93) higher circulating triglycerides and an odds ratio for myocardial infarction of 2.3 (95% CI 1.3-4.2). Furthermore, at least two variants associated with increased lipoprotein(a) were identified in 2.1% of myocardial infarction cases, with an odds ratio of 2.8 (95% CI 1.7-4.4) for myocardial infarction. Among 2,521 controls from the MESA cohort with lipoprotein(a) levels available, inheriting at least two variants known to increase lipoprotein(a) was associated with a 16.6 mg/dl (95% CI 4.7-29) higher circulating concentration.

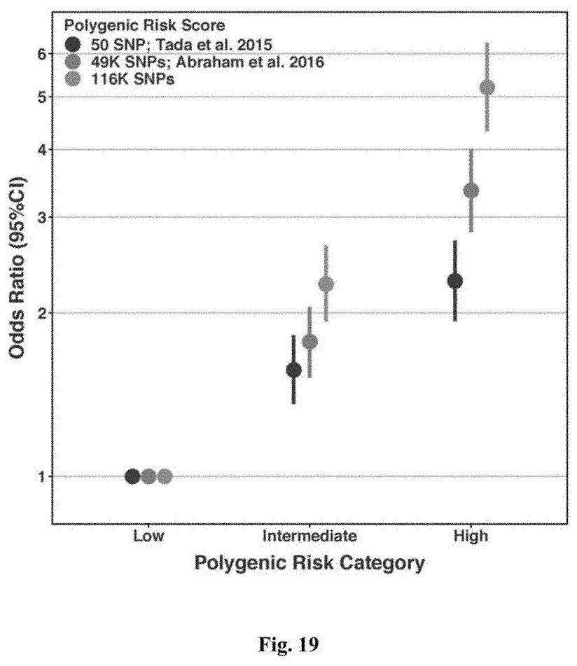

Applicants derived 24 distinct polygenic risk scores based on results from a previously published analysis with numbers of genetic variants in each score ranging from 78 to 2.04 million. Each of these scores was evaluated in an independent testing dataset of individuals from the UK Biobank (Table 32 & FIG. 23). A score based on 116,859 variants demonstrated the highest area under the curve for prediction of coronary artery disease in this testing dataset and this score was further evaluated in the whole genome sequencing dataset. This score was almost entirely independent of the 10-year risk of cardiovascular events as calculated by the ACC/AHA Pooled Cohorts Equations (Pearson's r=0.03 in MESA participants). Applicants considered individuals in the lowest race-specific quintile of the polygenic score as having low polygenic risk, quintiles 2-4 intermediate risk, and the top quintile as high risk as performed previously. (See Tada et al., Eur. Heart J., 2016; 37(6):561-7; Khera et al., N. Engl. J. Med., 2016; 375(24):2349-2358). Separation of the cohort into race-specific quintiles of this score noted a 5.20-fold (95% CI 4.32-6.28) risk gradient, significantly better than scores based on 50 variants (see Tada et al., Risk prediction by genetic risk scores for coronary heart disease is independent of self-reported family history, Eur. Heart J., 2016; 37(6):561-7; Khera et al., N. Engl. J. Med., 2016; 375(24):2349-2358) (risk gradient 2.30; 95% CI 1.93-2.73) or more than 49,000 variants (see Abraham et al., Eur. Heart J., 2016; 37(43):3267-3278) (risk gradient 3.38; 95% CI 2.83-4.02) (FIG. 19). In aggregate, 700 of 2369 (30%) of individuals with early-onset myocardial infarction were in the top quintile of the expanded polygenic risk score as compared to 617 of 4218 (15%) of controls.

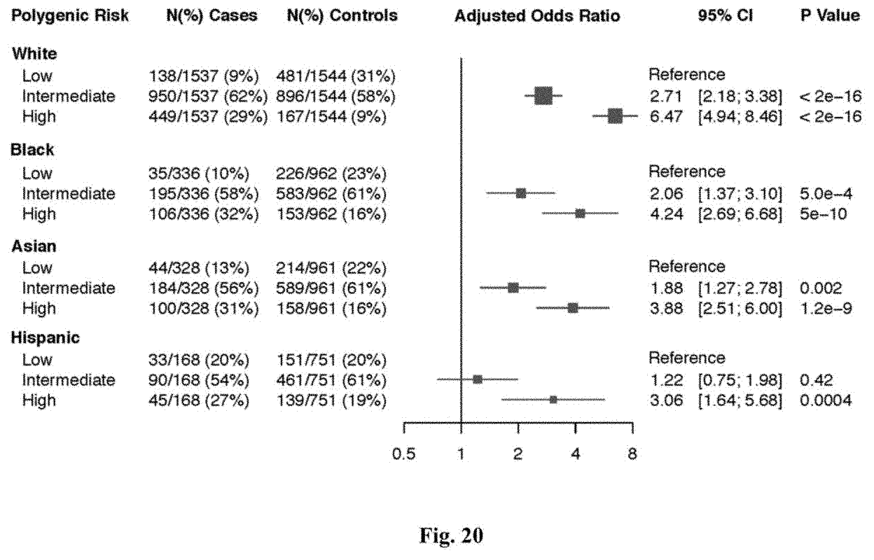

Importantly, the polygenic risk score was selected from 24 scores derived and validated based on a previously published GWAS and the UK Biobank, both of which were comprised primarily of participants of European ancestry. Applicants next tested the association of polygenic risk categories with myocardial infarction in subpopulations stratified by race. Although the score was robustly associated with risk within each group, the performance was best in white participants—6.5 fold (95% CI 5.0-8.5) risk gradient between those of low and high polygenic risk—as compared with gradients of 4.2 fold, 3.9 fold, and 3.1 fold in black, Asian, and Hispanic participants respectively (p-interaction=0.001; FIG. 20).

Applicants examined the quantitative importance and interplay of monogenic and polygenic risk pathways as they related to inherited risk of myocardial infarction. The risk associated with mutations in monogenic risk pathways was similar across strata of polygenic risk (p-interaction=0.08). Among the 2,369 individuals with myocardial infarction, 78 (3.3%) harbored a monogenic risk pathway mutation but were not in the top quintile of the polygenic risk score, 664 (28%) were in the top quintile of the polygenic risk score but did not harbor a monogenic risk pathway mutation, and 36 (1.5%) both harbored a monogenic pathway mutation and were in the top quintile of the polygenic score. As compared with those with no monogenic pathway mutation and low or intermediate polygenic risk, a monogenic risk pathway mutation or a high polygenic risk score each conferred a roughly three-fold increase in risk (OR 2.74 [95% CI 2.39-3.14] or 3.03 [95% CI 2.13-4.31], respectively). By contrast, those with both a monogenic pathway mutation and increased polygenic risk had a 5.88-fold (95% CI 3.20-11.09) increased risk of early-onset myocardial infarction.

### Discussion

In this study, Applicants compared the whole genome sequences of 2,369 individuals who suffered myocardial infarction at an early age with 4,218 control individuals free of cardiovascular disease. In a genetic association analysis, Applicants did not identify any new variants or genes associated with myocardial infarction. In a clinical interpretation framework integrating monogenic and polygenic risk pathways, Applicants observed a monogenic risk pathway mutation in 4.8% of individuals with early-onset myocardial infarction and these mutations conferred approximately three-fold increased risk. Applicants developed a new polygenic risk score of 116,859 genetic variants and this score demonstrated a 5.2-fold risk gradient across quintiles.

These results permit several conclusions of relevance to complex trait genetics. First, discovery of rare variant associations with disease in noncoding sequence is likely to require substantially increased sample sizes and improvements in the functional annotation of noncoding variants. Notably, the majority of observed variants reside in intergenic or intronic regions and are present in fewer than in 1 in 1,000 individuals. Our analysis of rare variation in regulatory sequences in tissues of known relevance to human atherosclerosis did not identify statistically significant associations.

Second, a mutation in a monogenic risk pathway was identified in 4.8% of sequenced individuals. These mutations are linked to impaired clearance of LDL cholesterol (familial hypercholesterolemia), defective triglyceride lipolysis, and increased lipoprotein(a). In aggregate, such mutations conferred a three-fold increased risk, broadly consistent with previous reports. (See Do et al., Exome sequencing identifies rare LDLR and APOA5 alleles conferring risk for myocardial infarction, Nature, 2015; 518(7537):102-6; Khera et al., Association of rare and common variation in the lipoprotein lipase gene With coronary artery disease, JAMA, 2017; 317(9):937-946; Khera et al., Diagnostic yield and clinical utility of sequencing familial hypercholesterolemia genes in patients with severe hypercholesterolemia, J. Am. Coll. Cardiol., 2016; 67(22):2578-89; Clarke et al., Genetic variants associated with Lp(a) lipoprotein level and coronary disease, N. Engl. J. Med., 2009; 361(26):2518-28; Abul-Husn et al., Genetic identification of familial hypercholesterolemia within a single U.S. health care system, Science, 2016; 354(6319)). Importantly, each of these driving pathways can be targeted using potent therapeutics currently available or in development—statins, ezetimibe, and drugs targeting PCSK9 (monoclonal antibodies or RNA interference) to reduce LDL cholesterol, an antisense oligonucleotide targeting apolipoprotein C-III to accelerate triglyceride clearance, and an antisense oligonucleotide to lower lipoprotein(a). (See Sabatine et al., Evolocumab and clinical outcomes in patients with cardiovascular disease, N. Engl. J. Med., 2017 May 4; 376(18):1713-1722; Gaudet et al., Antisense inhibition of apolipoprotein C-III in patients with hypertriglyceridemia, N. Engl. J. Med., 2015; 373(5):438-47; Viney et al., Antisense oligonucleotides targeting apolipoprotein(a) in people with raised lipoprotein(a): two randomised, double-blind, placebo-controlled, dose-ranging trials, Lancet, 2016; 388(10057):2239-2253).

Third, inheritance of a disproportionate number of common genetic risk variants, each with a modest impact, represents another mechanism underlying genetic predisposition. Monogenic risk pathways and this polygenic risk contributed to risk of myocardial infarction in an additive fashion. Applicants derived and validated a new polygenic risk score that includes 116,859 genetic variants scattered across the genome. This expanded score significantly outperformed previous such scores with a more than five-fold risk gradient observed across score quintiles. (See Tada et al., Risk prediction by genetic risk scores for coronary heart disease is independent of self-reported family history, Eur. Heart J., 2016; 37(6):561-7; Khera et al., Genetic risk, adherence to a healthy lifestyle, and coronary disease, N. Engl. J. Med., 2016; 375(24):2349-2358; Abraham et al., Genomic prediction of coronary heart disease, Eur. Heart J., 2016; 37(43):3267-3278). However, consistent with the development and validation of this and previous scores in individuals of European ancestry, significant heterogeneity in score performance was noted across racial subgroups. (See Martin et al., Human demographic history impacts genetic risk prediction across diverse populations, Am. J. Hum. Genet., 2017; 100(4):635-649). Evidence derived from randomized clinical trials suggests that those with increased polygenic risk derive increased absolute and relative coronary risk reduction with statin therapy. (See Mega et al., Genetic risk, coronary heart disease events, and the clinical benefit of statin therapy: an analysis of primary and secondary prevention trials, Lancet, 2015; 385(9984):2264-71; Natarajan et al., Polygenic risk score identifies subgroup with higher burden of atherosclerosis and greater relative benefit from statin therapy in the primary prevention setting, Circulation, 2017 Feb. 21. [Epub ahead of print]). Similarly, absolute risk reductions associated with adherence to a healthy lifestyle were highest in the high genetic risk subgroup. (See Khera et al., Genetic risk, adherence to a healthy lifestyle, and coronary disease, N. Engl. J. Med., 2016; 375(24):2349-2358). Ascertainment of polygenic risk for common diseases may thus facilitate intensive prevention efforts via lifestyle or pharmacotherapy.

In conclusion, after assessment of more than 145 million genetic variants in 6,587 individuals of a multiethnic case-control study, Applicants identify both mutations in monogenic risk pathways and polygenic risk as important contributors to the genetic underpinnings of early-onset myocardial infarction.

### REFERENCES

Gertler M M, Garn S M, White P D. Young candidates for coronary heart disease. J Am MedAssoc. 1951; 147(7):621-5.
Lehrman M A, Schneider W J, Sidhof T C, Brown M S, Goldstein J L, Russell D W. Mutation in LDL receptor: Alu-Alu recombination deletes exons encoding transmembrane and cytoplasmic domains. Science. 1985; 227(4683):140-6.
Samani N J, Erdmann J, Hall A S, et al. Genomewide association analysis of coronary artery disease. N Engl J Med. 2007; 357:443-53.
Helgadottir A, Thorliefsson G, Manolescu A, et al. A common variant on chromosome 9p21 affects the risk of myocardial infarction. Science. 2007; 316:1491-1493.
McPherson R, Pertsemlidis A, Kavaslar N, et al. A common allele on chromosome 9 associated with coronary heart disease. Science. 2007; 316:1488-1491.
Myocardial Infarction Genetics Consortium, Kathiresan S, Voight B F, et al. Genome-wide association of early-onset myocardial infarction with single nucleotide polymorphisms and copy number variants. Nat Genet. 2009; 41(3):334-41.
CARDIoGRAMplusC4D Consortium, Deloukas P, Kanoni S, et al. Large-scale association analysis identifies new risk loci for coronary artery disease. Nat Genet. 2013; 45:25-33.
Nikpay M, Goel A, Won H H, et al. A comprehensive 1,000 Genomes-based genome-wide association meta-analysis of coronary artery disease. Nat Genet. 2015; 47(10):1121-30.
Myocardial Infarction Genetics and CARDIoGRAM Exome Consortia Investigators, Stitziel N O, Stirrups K E, et al. Coding Variation in ANGPTL4, LPL, and SVEP1 and the Risk of Coronary Disease. N Engl J Med. 2016; 374(12):1134-44.
Webb T R, Erdmann J, Stirrups K E, et al. Systematic evaluation of pleiotropy identifies 6 further loci associated with coronary artery disease. J Am Coll Cardiol. 2017; 69(7):823-836.
Do R, Stitziel N O, Won H-H, et al. Exome sequencing identifies rare LDLR and APOA5 alleles conferring risk for myocardial infarction. Nature. 2015; 518(7537):102-6.
Cohen J C, Boerwinkle E, Mosley T H Jr, Hobbs H H. Sequence variations in PCSK9, low LDL, and protection against coronary heart disease. N Engl J Med. 2006; 354(12):1264-72.
Myocardial Infarction Genetics Consortium Investigators, Stitziel N O, Won H H, et al. Inactivating mutations in NPC1L1 and protection from coronary heart disease. N Engl J Med. 2014; 371(22):2072-82.
Nioi P, Sigurdsson A, Thorleifsson G, et al. Variant ASGR1 Associated with a Reduced Risk of Coronary Artery Disease. N Engl J Med 2016; 374(22):2131-41.
Jorgensen A B, Frikke-Schmidt R, Nordestgaard B G, Tybjerg-Hansen A. Loss-of-function mutations in APOC3 and risk of ischemic vascular disease. N Engl J Med 2014 Jul. 3; 371(1):32-41.
Crosby J, Peloso G M, Auer P L, et al. Loss-of-function mutations in APOC3, triglycerides, and coronary disease. N Engl J Med 2014; 371:22-31.
Dewey F E, Gusarova V, O'Dushlaine C O, et al. Inactivating variants in ANGPTL4 and risk of coronary artery disease. N Engl J Med 2016; 374(12):1123-33.
Khera A V, Won H H, Peloso G M, et al. Association of rare and common variation in the lipoprotein lipase gene With coronary artery disease. JAMA. 2017; 317(9):937-946.
Ashley E A. Towards precision medicine. Nat Rev Genet. 2016; 17(9):507-22.
Lichtman J H, Lorenze N P, D'Onofrio G, et al. Variation in recovery: Role of gender on outcomes of young AMI patients (VIRGO) study design. Circ Cardiovasc Qual Outcomes. 2010; 3(6):684-93.
Assimes T L, Lee I T, Juang J M, et al. Genetics of coronary artery disease in Taiwan: A cardiometabochip study by the Taichi Consortium. PLoS One. 2016; 11(3):e0138014.
Bild D E, Bluemke D A, Burke G L, et al. Multi-ethnic study of atherosclerosis: objectives and design. Am J Epidemiol. 2002; 156:871-881.
Landrum M J, Lee J M, Riley G R, et al. ClinVar: public archive of relationships among sequence variation and human phenotype. Nucleic Acids Res. 2014 January; 42(Database issue):D980-5.
Roadmap Epigenomics Consortium, Kundaje A, Meuleman W, et al. Integrative analysis of 111 reference human epigenomes. Nature. 2015; 518(7539):317-30.
Khera A V, Won H H, Peloso G M, et al. Diagnostic yield and clinical utility of sequencing familial hypercholesterolemia genes in patients with severe hypercholesterolemia. J Am Coll Cardiol. 2016; 67(22):2578-89.
Clarke R, Peden J F, Hopewell J C, et al. Genetic variants associated with Lp(a) lipoprotein level and coronary disease. N Engl J Med. 2009; 361(26):2518-28.
Tada H, Melander O, Louie J Z et al. Risk prediction by genetic risk scores for coronary heart disease is independent of self-reported family history. Eur Heart J. 2016; 37(6):561-7.
Khera A V, Emdin C A, Drake I, et al. Genetic risk, adherence to a healthy lifestyle, and coronary disease. N Engl J Med. 2016; 375(24):2349-2358.
Abraham G, Havulinna A S, Bhalala O G, et al. Genomic prediction of coronary heart disease. Eur Heart J. 2016; 37(43):3267-3278.
The 1000 Genomes Project Consortium. A global reference for human genetic variation. Nature. 2015; 526(7571):68-74.
Sudlow C, Gallacher J, Allen N, et al. U. K. Biobank: an open access resource for identifying the causes of a wide range of complex diseases of middle and old age. PLoS Med 2015; 12:e1001779.
Pulit S L, de With S A, de Bakker P I. The multiple testing burden in sequencing-based disease studies of global populations. bioRxiv 053264; doi: doi.org/10.1101/053264.
Abul-Husn N S, Manickam K, Jones L K, et al. Genetic identification of familial hypercholesterolemia within a single U.S. health care system. Science. 2016; 354(6319) doi: 10.1126/science.aaf7000.
Sabatine M S, Giugliano R P, Keech A C, et al. Evolocumab and clinical outcomes in patients with cardiovascular disease. N Engl J Med. 2017 May 4; 376(18):1713-1722.
Gaudet D, Alexander V J, Baker B F, et al. Antisense inhibition of apolipoprotein C-III in patients with hypertriglyceridemia. N Engl J Med. 2015; 373(5):438-47.
Viney N J, van Capelleveen J C, Geary R S, et al. Antisense oligonucleotides targeting apolipoprotein(a) in people with raised lipoprotein(a): two randomised, double-blind, placebo-controlled, dose-ranging trials. Lancet. 2016; 388(10057):2239-2253
Martin A R, Gignoux C R, Walters R K, et al. Human demographic history impacts genetic risk prediction across diverse populations. Am J Hum Genet. 2017; 100(4):635-649.
Mega J L, Stitziel N O, Smith J G, et al. Genetic risk, coronary heart disease events, and the clinical benefit of statin therapy: an analysis of primary and secondary prevention trials. Lancet. 2015; 385(9984):2264-71.
Natarajan P, Young R, Stitziel N O, et al. Polygenic risk score identifies subgroup with higher burden of atherosclerosis and greater relative benefit from statin therapy in the primary prevention setting. Circulation. 2017 Feb. 21. [Epub ahead of print]
McLaren W, Gil L, Hunt S E, Riat H S, et al. The Ensembl Variant Effect Predictor. Genome Biol. 2016; 17(1):122.

### Example 4

Polygenic risk scores provide a quantitative metric of an individuals inherited risk based on the cumulative impact of many variants. Weights are generally assigned to each genetic variant according to the strength of their association with disease risk (effect estimate). Individuals are scored based on how many risk alleles they have for each variant (e.g. 0, 1, 2 copies) included in the polygenic risk score.

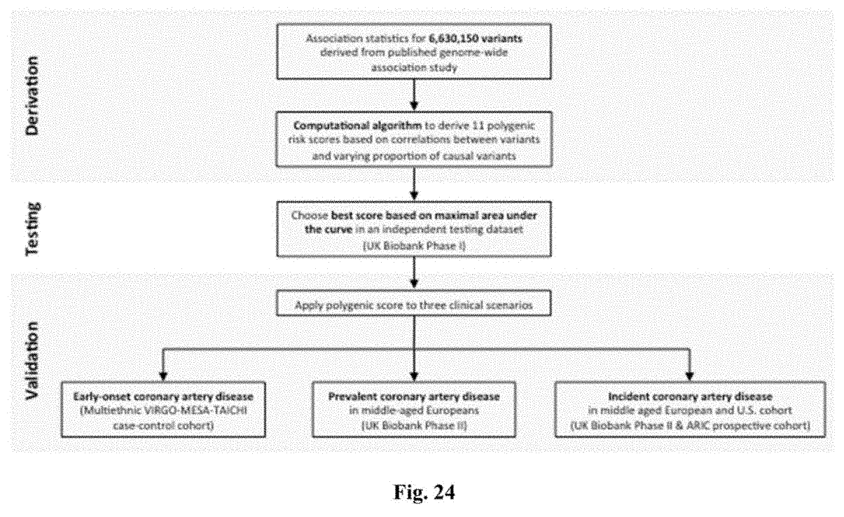

Polygenic risk can be quantified by assessing the number of risk variants in each individual, weighted by the impact of each variant on disease. Here, previously published data for the association of 6.6 million common genetic variants with coronary artery disease (CAD) were used to derive several polygenic scores (FIG. 24). Second, a testing dataset was used to choose the best score. Third, this score was applied to independent validation datasets representing three clinical scenarios—a multiethnic case-control cohort of early-onset CAD (age <60 years), prevalent CAD in a middle-aged European cohort, and incident CAD in middle-aged European and United States prospective cohorts.

### Polygenic Score Derivation and Testing:

A genome-wide polygenic score was derived based on the association statistics of all available common (minor allele frequency ≥0.01) single nucleotide polymorphisms with CAD, as determined by a published genome-wide association study of 60,801 individuals with CAD and 123,504 controls.¹⁶ The inter-relationship between these variants was assessed using a reference population of 503 Europeans from the 1000 Genomes study.¹⁷

The LDPred computational algorithm was then used to construct polygenic scores. Vilhjálmsson, B. J. et al. Am J Hum Genet. 2015; 97:576-92 (2015). LDpred creates a polygenic risk score using genome-wide variation with weights derived from a set of GWAS summary statistics. Unlike other methods that use variants most strongly associated with disease risk or a set of independent variants across the genome, LDpred includes all available variants in the derived risk score by shrinking effect estimate weights (log-odds) based on an external LD reference panel. This Bayesian approach calculates a posterior mean effect size for each variant based on a prior (association with CAD in a previously published study) and subsequent shrinkage based on the extent to which this variant is correlated with similarly associated variants in a reference population. The underlying Gaussian distribution additionally considers the fraction of causal (e.g. non-zero effect sizes) markers. Because this fraction is unknown for any given disease, LDpred uses a range of plausible values to construct eleven different polygenic scores. For score derivation, CAD summary statistics from a comprehensive 1000 Genomes imputed GWAS of primarily European individuals (CARDIoGRAMplusC4D Consortium, Am J Hum Genet. 97(4), 576-92 (2015)) and a linkage disequilibrium reference panel of 503 European samples from 1000 Genomes phase 3 version 5 (The 1000 Genomes Project Consortium, A global reference for human genetic variation, Nature, 526(7571):68-74 (2015)) were used. Single Nucleotide Polymorphisms (SNPs) with ambiguous strand (A/T or C/G) or minor allele frequency less than 1% were removed from the score derivation. This left 6,630,150 variants available for inclusion. In accordance with recommendations from the LDpred authors, a linkage dysequilibrium radius was set at 2210 variants, equivalent to the number of SNPs used as input divided by 3000. A range of p, the fraction of causal variants, was used—1, 0.5, 0.03, 0.01, 0.003, 0.001, 0.0003, 0.0001—along with an infinitesimal (See Visscher, P. M. et al, Nat Rev Genet. 9(4):255-66. (2008)) (each variant assumed to contribute to disease risk) and unweighted model (raw log-odds for all variants input) were considered.

Choosing Best Polygenic Risk Score Based on Testing Dataset Performance.

The best score was then determined based on maximal area under the curve from logistic regression models in a previously described CAD case-control cohort of 120,286 individuals (4,831 European CAD cases and 115,455 European controls) from the UK Biobank phase I cohort. (See Klarin, D. et al. Nat Genet. Jul. 17, 2017, doi: 10.1038/ng.3914 [Epub ahead of print]).

Scores were generated by multiplying the genotype dosage of each risk allele for each variant by its respective weight, and then summing across all variants in the score. Incorporating genotype dosages accounts for uncertainty in genotype imputation. All calculations were performed using Hail (github.com/hail-is/hail). Over 99.9% of variants in the LDpred-derived risk scores were available for scoring purposes in the UK Biobank phase I genotype release with sufficient imputation quality (INFO >0.3).

The association between each PRS and CAD status was determined using logistic regression, adjusted for the first four principal components of ancestry. Area under the curve (AUC) was used to determine model discrimination. While most PRS showed a highly significant association with CAD status, the PRS generated by LDpred with p=0.001 showed the best discrimination based on AUC (Table 34).

TABLE 34
Performance of LDpred-derived polygenic scores in the testing dataset.

| | | # SNPs (%) | | | |
|---|---|---|---|---|---|
| | #SNPs in | UK Biobank Phase I | | | |
| | Polygenic | Genotype Release | | | OR_{Q5}_v_Q1 |
| Polygenic Score | Score | INFO >.3 | | AUC | (95% CI) |
| Unweighted | 6,630,150 | 6,629,369 | (99.9%) | 0.597 | 2.59 (2.35-2.86) |
| LDpred-inf | 6,630,150 | 6,629,369 | (99.9%) | 0.599 | 2.67 (2.42-2.95) |
| LDpred ρ = 1 | 6,630,150 | 6,629,369 | (99.9%) | 0.608 | 2.98 (2.70-3.29) |
| LDpred ρ = 0.3 | 6,630,150 | 6,629,369 | (99.9%) | 0.608 | 2.99 (2.71-3.30) |
| LDpred ρ = 0.1 | 6,630,150 | 6,629,369 | (99.9%) | 0.610 | 3.05 (2.76-3.37) |
| LDpred ρ = 0.03 | 6,630,150 | 6,629,369 | (99.9%) | 0.615 | 3.19 (2.88-3.52) |
| LDpred ρ = 0.01 | 6,630,150 | 6,629,369 | (99.9%) | 0.623 | 3.42 (3.09-3.79) |
| LDpred ρ = 0.003 | 6,630,150 | 6,629,369 | (99.9%) | 0.635 | 3.92 (3.53-4.35) |
| LDpred ρ = 0.001 | 6,630,150 | 6,629,369 | (99.9%) | 0.640 | 4.09 (3.68-4.54) |
| LDpred ρ = 0.0003 | 6,630,150 | 6,629,369 | (99.9%) | 0.515 | 1.10 (1.00-1.20) |
| LDpred ρ = 0.0001 | 6,630,150 | 6,629,369) | (99.9% | 0.511 | 1.09 (0.99-1.19) |
| Khera et al.²⁷ | 50 | 50 | (100%) | 0.593 | 2.47 (2.24-2.72) |
| Abraham et al.²⁸ | 49,305 | 49,170 | (99.7%) | 0.590 | 2.48 (2.25-2.73) |

### Validation Study Populations:

A multiethnic early-onset (age <60 years) CAD case-control cohort was assembled using cases from the previously described Variation in Recovery: Role of Gender on Outcomes of Young AMI Patients (VIRGO) and TAICHI consortium and controls from the Multi-Ethnic Study of Atherosclerosis (MESA) cohort and TAICHI consortium. The design of the Variation in Recovery: Role of Gender on Outcomes of Young AMI Patients (VIRGO) study has been previously described.⁷ The VIRGO study enrolled a multiethnic population of adult patients in the United States and Spain with a first myocardial infarction at age ≤55 years. (See Lichtman, J. H. et al., Circ Cardiovasc Qual Outcomes, 3, 684-93 (2010)) In brief, 3,501 participants hospitalized with an acute myocardial infarction, age 18 to 55 years, were enrolled between 2009 and 2012 from 103 United States and 24 Spanish hospitals using a 2:1 female-to-male enrollment design. Baseline patient data were collected by medical chart abstraction and standardized in-person patient interviews administered by trained personnel during the index acute myocardial infarction admission. Individuals with available DNA, all of whom were derived from United States enrollment centers, and who had provided written informed consent for genetic analysis were included in the present study.

The TAICHI consortium enrolled patients with an early-onset coronary event (men ≤50 years, women ≤60 years) in the context of normal circulating lipid levels (LDL cholesterol <130 mg/dl or total cholesterol <185 mg/dl) and controls in Taiwan. (See Assimes, T. L. et al., PLoS One, 0.11, e01380142016 (2016)) Individuals with coronary disease were identified as those with a history of myocardial infarction, coronary revascularization, or a stenosis of ≥50% in a major epicardial vessel demonstrated by angiography. All cases experienced an early-onset coronary event (men ≤50 years, women ≤60 years) in the context of normal circulating lipid levels (LDL cholesterol <130 mg/dl or total cholesterol <185 mg/dl). Controls were enrolled from an epidemiology study and from the several Hospital Endocrinology and Metabolism Departments either as outpatients or as their family members. Subjects with a history of CAD were excluded.

The MESA study is a multiethnic prospective cohort that enrolled individuals in the United States free of cardiovascular disease between 2000 and 2002. The design of the MESA study has been previously described and protocol available at www.mesa-nhlbi.org. (See, Bild, D. E. et al., Am J. Epidemiol.; 156, 871-881 (2002). In brief, 6,181 men and women between the ages of 45 and 84 without prevalent cardiovascular disease were recruited between 2000-2002 from 6 United States communities. Individuals were excluded from the present study due if informed consent for genetic testing had not been obtained/was withdrawn, DNA was not available for sequencing, or incident cardiovascular disease (myocardial infarction, coronary revascularization, angina, peripheral arterial disease, stroke, resuscitated cardiac arrest, death due to cardiovascular causes) through the period of last available follow-up in December 2014. Fasting plasma triglyceride, total cholesterol, high density lipoprotein cholesterol (HDL-C) concentrations were measured as described previously. (See Tsai, M. Y. et al., Atherosclerosis 200, 359-67 (2008)). Low density lipoprotein-cholesterol (LDL-C) was calculated based on the Friedewald formula in participants with triglycerides <400 mg/dL. (See Friedewald, W. T. et al., Clin Chem 18(6), 499-502 (1972).

MESA participants were included as controls for this study if they remained free of incident cardiovascular disease through the end of 2014 (median follow-up 13.2 years). The polygenic score calculation was calculated based on whole genome sequencing data. Because the polygenic score was derived and tested based on studies comprised primarily of participants of European ancestry, Applicants determined whether the association of the polygenic score with early-onset CAD varied according to race or ethnicity.

Genotypes in the VIRGO-MESA-TAICHI were ascertained using whole genome sequencing, performed at the Broad Institute of Harvard and MIT (Cambridge, Mass., USA). Libraries were constructed and sequenced on the Illumina HiSeqX with the use of 151-bp paired-end reads for whole-genome sequencing. Output from Illumina software was processed by the Picard data-processing pipeline to yield BAM files containing well-calibrated, aligned reads. All sample information tracking was performed by automated LIMS messaging. A sample was considered sequence complete when the mean coverage was ≥30× (for the MESA cohort) or ≥20× (for VIRGO and TAICHI cohorts). Two quality control metrics that are reviewed along with the coverage are the sample Fingerprint LOD score and % contamination. At aggregation, an all-by-all comparison was done of the read group data and estimate the likelihood that each pair of read groups is from the same individual. If any pair had a LOD score <−20.00, the aggregation does not proceed and is investigated. FP LOD > or =3 is considered passing concordance with the sequence data (ideally LOD >10). A sample will have an LOD of 0 when the sample failed to have a passing fingerprint. Fluidigm fingerprint is repeated once if failed. Read groups with fingerprints <−3.00 were blacklisted from the aggregation. Sample genotypes were determined via a joint callset using the Genome Analysis Toolkit Haplotype Caller.

6,809 individuals underwent whole genome sequencing, of whom 222 (3.3%) were excluded based on sequencing quality control metrics (Table 35). Sample exclusion criteria included:
1. DNA Contamination >5%
2. Mean coverage <20×
3. Sample duplicates/Identical Twins (as assessed by PI_HAT≥0.95)
4. First or second degree relatives of another study participant (Kinship coefficient >0.0884)
5. Variant Call Rate <95%
6. Genotype/phenotype Sex Discordance or ambiguous sex (0.5<Fₛₜₐₜ<0.8)

TABLE 35
Sample Quality Control Criteria in the VIRGO-MESA-TAICHI Validation Cohort

| | Thresholds | MESA | VIRGO | TAICHI | Total |
|---|---|---|---|---|---|
| Initial Sample Size | | 3932 | 2101 | 776 | 6809 |
| Contamination | >5.0 % | 19 | 3 | 0 | 22 |
| Raw Mean | <20X | 1 | 2 | 1 | 4 |
| Coverage | | | | | |
| Duplicates/Twins | PI-Hat ≥0.95 | 2 | 10 | 3 | 15 |
| 1^{st}/2^{nd} Degree | Kinship | 148 | 2 | 2 | 152 |
| Relatives | Coefficient >0.0884 | | | | |
| Post-QC Call Rate | <95% | 0 | 3 | 18 | 21 |
| Sex Check | 0.5 < Fstat < 0.8 | 1 | 0 | 7 | 8 |

| | | | | |
|---|---|---|---|---|
| Total Cases | 0 | 2081 | 288 | 2369 |
| Total Controls | 3761 | 0 | 457 | 4218 |

| | |
|---|---|
| Total Sample Size | 6587 |

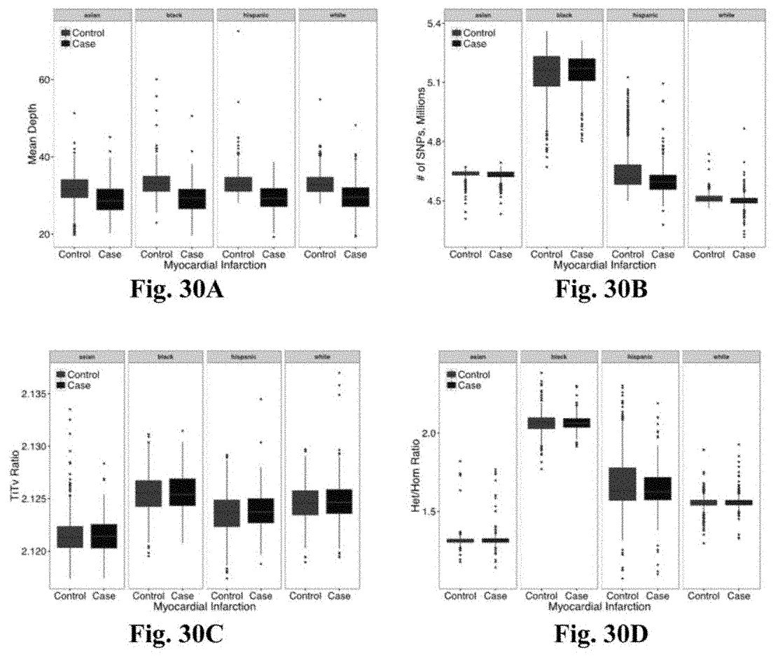

Baseline characteristics of the 6,587 remaining individuals, stratified by early-onset coronary artery disease case versus control status, are provided in Table 36. Principal components analysis demonstrated that cases and controls were well-matched according to genetic ancestry. Mean sequencing depth was 31.7× (SD 3.8) across the study cohorts with similar quality metrics observed across cases and controls (FIG. 30).

TABLE 36
Baseline Characteristics of Study Participants in the
VIRGO-MESA-TAICHI Early-onset
Coronary Artery Disease Validation Dataset

| | Early-Onset | |
|---|---|---|
| | CAD Cases | Controls |
| | N = 2369 | N = 4218 |
| Study | | |
| MESA | 0 | 3761 (89%) |
| VIRGO | 2081 (88%) | 0 |
| TAICHI | 288 (12%) | 457 (11%) |
| Race | | |
| White | 1537 (65%) | 1544 (37%) |
| Black | 336 (14%) | 962 (23%) |
| Asian | 328 (14%) | 961 (23%) |
| Hispanic | 168 (7%) | 751 (18%) |
| Male | 925 (39%) | 2019 (48%) |
| Age, years; Mean (SD) | 48 (6) | 61 (10) |
| Hypertension | 1415 (60%) | 1600 (38%) |
| Diabetes | 876 (37%) | 665 (16%) |
| Current Smoking | 1146 (49%) | 535 (13%) |
| Statin Use | 668 (29%) | 584 (14%) |
| Lipid Levels, Mean (SD) | | |
| LDL Cholesterol, | 110 (41) | 116 (38) |
| mg/dl | | |
| HDL Cholesterol, | 41 (13) | 51 (15) |
| mg/dl | | |
| Triglycerides, mg/dl | 182 (205) | 132 (82) |

In order to assign race within this cohort, A panel of approximately 16,000 ancestry informative markers (AIMs) (see Hoggart, C. J. et al., Am J Hum Genet 72(6), 1492-1504 (2003) identified across six continental populations was chosen to derive principal components (PCs) of ancestry for all samples that passed quality control. Principal component analysis was performed using EIGENSTRAT. (See Price, A. L. et al., Nat Genet 38, 904-9 (2006).

In order to assign a race to individuals without self-reported race or with discordant self-reported race and PC ancestry, a k-nearest neighbors (k-NN) classifier (see Fix, E. et al., Texas: USAF School of Aviation Medicine, pp 261-279 (1951); Cover, T. et al., IEEE Trans Inf Theory. 13, 21-27 (1967)) was applied using the first five PCs of ancestry. This analysis was done using the k-NN implementation from the Scikit-learn library in Python. (See Pedregosa, F. et al., Journal of Machine Learning Research.; 12, 2825-30 (2011)) The classifier was built using MESA samples after removing 25 individuals with discordant self-reported race and PC ancestry as determined by visual inspection of PC1 and PC2. The remaining MESA samples were split into a training set (n=2490) and test set (n=1246). A k-NN (k=5) classifier was built using self-reported race as the dependent variable (1: White/Caucasian, 2: Chinese American, 3: Black/African-American, 4: Hispanic) and PC1 to PC5 as features. The classifier had a 98.1% reclassification rate in the test set, with misclassifications generally occurring for Hispanic individuals. This classifier was then applied to all 6587 samples to generate inferred race.

A second validation set for prevalent and incident CAD was assembled from individuals of European ancestry from the UK Biobank phase II cohort. (See Sudlow, C. et al., PLos Med 12, e1001779 (2015)). The UK Biobank enrolled individuals aged 45 to 69 years old from across the United Kingdom beginning in 2006. Individuals who self-reported a history of myocardial infarction or coronary revascularization or were hospitalized for acute myocardial infarction or coronary revascularization in the electronic health record prior to enrollment were considered prevalent cases; all other individuals were considered controls. Incident coronary events were ascertained based on hospital admission for an acute myocardial infarction or coronary revascularization or fatal CAD as detected in the death registry.

Individuals in the UK Biobank underwent genotyping with one of two closely related custom arrays (UK BiLEVE Axiom Array or UK Biobank Axiom Array) consisting of over 800,000 genetic markers scattered across the genome. (See Bycroft et al., bioRxiv, doi.org/10.1101/166298 (2017)). Additional genotypes were imputed centrally using the Haplotype Reference Consortium and UK10K haplotype resource where available and the 1000 Genomes Phase 3 reference panel otherwise to generate imputation results. In order to analyze individuals with a relatively homogenous ancestry and owing to small percentages of non-British individuals, the present analysis was restricted to the white British ancestry individuals. This subpopulation was constructed centrally using a combination of self-reported ancestry and genetically confirmed ancestry using principal components. Additional exclusion criteria included outliers for heterozygosity or genotype missingness, discordant reported versus genotypic sex, putative sex chromosome aneuploidy, or withdrawal of informed consent. Each of these parameters was derived centrally as previously reported. (Bycroft, C. et al., 2017).

Baseline characteristics of the 288,980 remaining individuals for the prevalent coronary artery disease analysis are provided in Table 37. Current smoking, lipid lowering-medication, and parental history of heart disease was determined by self-report at the time of enrollment survey. Diabetes mellitus, hypertension, and dyslipidemia were assessed based on a combination of self-report or hospitalization diagnosis code prior to date of UK Biobank enrollment reflecting these conditions.

TABLE 37
Baseline Characteristics of the UK Biobank Phase II
Prevalent CAD Cohort

| | | | CAD-Free | | |
|---|---|---|---|---|---|
| | CAD Cases | | Controls | | |
| | N = 8,676 | | N = 280,304 | | P-value |
| Age, years | 62 | (6) | 57 | (8) | <0.001 |
| Male Gender | 6,953 | (80%) | 124,130 | (44%) | <0.001 |
| Hypertension | 5,701 | (66%) | 75,758 | (27%) | <0.001 |
| Diabetes Mellitus | 1,582 | (18.2%) | 12,406 | (4%) | <0.001 |
| Dyslipidemia | 5,601 | (65%) | 34,000 | (12%) | <0.001 |
| Current Smoking | 1,079 | (12%) | 25,520 | (9%) | <0.001 |
| Family History of Heart | 4,184 | (48%) | 100,036 | (36%) | <0.001 |
| Disease | | | | | |
| Body-mass Index, kg/m² | 29.3 | (4.8) | 27.3 | (4.7) | <0.001 |
| Lipid-lowering Medication | 7,724 | (90%) | 41,788 | (15%) | <0.001 |

Values represent N (% with non-missing values), mean (SD), or median (IQR). P-values computed via ANOVA for continuous variables (TG modeled using Kruskal-Wallis test) and chi-square test for categorical variables.

Diagnosis of prevalent coronary artery disease was based on a composite of myocardial infarction or coronary revascularization. Myocardial infarction was based on self-report or hospital admission diagnosis, as performed centrally. (See Schnier, C. et al., Definitions of acute myocardial infarction (MI) and main MI pathological types for UK Biobank phase 1 outcomes adjudication; Version 1, January 2017. Available at: biobank.ctsu.ox.ac.uk/crystal/refer.cgi?id=461). This included individuals with ICD-9 codes of 410.X, 411.0, 412.X, 429.79 or ICD-10 codes of I21.X, I22.X, I23.X, 124.1, 125.2 in hospitalization records. Among the 280,304 individuals free of prevalent coronary artery disease at baseline, incident events included myocardial infarction, fatal coronary event, and coronary revascularization. Myocardial infarction was ascertained using the above ICD-10 diagnoses codes in hospitalization records or the death registry as an underlying cause of death. Coronary revascularization, inclusive of percutaneous angioplasty or coronary artery bypass surgery, was extracted from OPCS (Office of Population, Censuses and Surveys: Classification of Interventions and Procedures) hospitalization procedure codes.

Individuals without evidence of an incident event were censored at the earlier of last hospitalization or death registry follow-up. This corresponded to February 2016 for England and Wales and October 2016 for Scotland participants.

The polygenic score calculation was calculated using array-based genotyping and imputation. (Bycroft, C. et al., 2017).

The third validation study for incident events involved white participants free of prevalent CAD from the Atherosclerosis Risk in Communities (ARIC) study, a prospective cohort that enrolled participants between the ages of 45 and 64 years starting in 1987. (Am J Epidemiol., 129, 687-702 (1989). The ARIC study is a prospective cohort with emphasis on the epidemiology of cardiovascular disease. Baseline lipid levels were measured in the ARIC central lipid laboratory using commercial reagents. (See Brown, S. A. et al. Arterioscler Thromb 13, 1139-58 (1993)). Genotype and clinical data were retrieved from the National Center for Biotechnology Information dbGAP server (accession: phs000280.v3.p1).

Genotyping was performed using the Affymetrix 6.0 array (Affymetrix, Santa Clara, Calif.) and subsequently imputed to the Haplotype Reference Consortium using the Michigan Imputation Server. (See Das, S. et al., Nat Genet 48, 1279-83 (2016)). Phasing was performed using the Eagle2 algorithm. (See Loh, P. R. et al., Nat Genet.; 48, 1443-8 (2016)). 4,954 variants were removed prior to imputation due to duplication, monomorphism or allele mismatch. Imputation was then performed on 799,246 variants using the minimac3 algorithm and the Haplotype Reference Consortium reference panel. (Loh, P. R. et al., 2016). Individuals were excluded if they had prevalent coronary artery disease at the time of enrollment, were outliers with respect to principal components of ancestry, or were related to another individual in the cohort. A composite CAD endpoint including myocardial infarction, coronary revascularization, and death from coronary causes was used in this study. Endpoint adjudication was performed by committee review of medical records for reported endpoints. (See ARIC manual of operations. No. 2. Cohort component procedures. Chapel Hill: University of North Carolina, ARIC Coordinating Center, School of Public Health, 1987). The polygenic score calculation was based on array-based genotyping data and subsequent imputation.

### Statistical Analysis

Within each cohort, individuals were categorized as having low (bottom quintile), intermediate (quintiles 2-4), or high (top quintile) polygenic risk. See Khera et al., N Engl J Med, 375, 2349-58 (2016)). The relationship of these categories to prevalent CAD was determined using logistic regression, adjusting for principal components of ancestry. Principal components of ancestry are based on observed genotypic differences across individuals; their inclusion as covariates in regression analyses minimizes confounding by ancestry. (Price, A. L. et al., 2006). All UK Biobank validation analyses additionally included genotyping array indicator variable in regression models. (Bycroft, C. et al., 2017). The association of the polygenic scores with incident events was determined by calculation of absolute incidence rates and subsequent Cox regression analyses adjusted for age, gender, traditional cardiovascular risk factors or scores, and principal components of ancestry as covariates. Discrimination was assessed using C-statistics and reclassification using the net reclassification index. (See, Pencina, M. J. et al., Stat Med, 27, 157-72 (2008). Tests of interaction between the polygenic score and traditional risk factors were performed within Cox regression analyses adjusted for age, gender, and principal components of ancestry.

Analyses were performed using R version 3.2.2 software (The R Foundation).

### Results.

### Polygenic Score Derivation & Selection

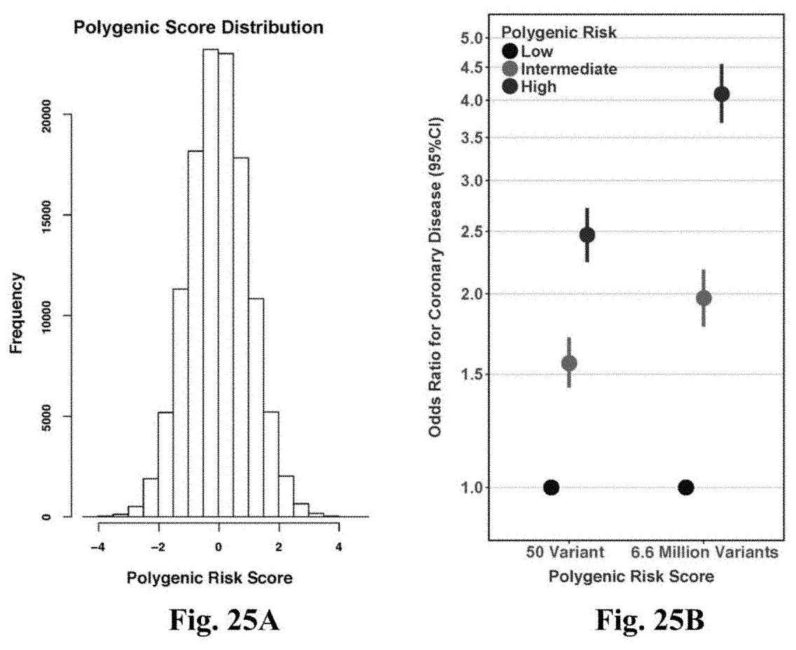

Using the association statistics of 6,630,150 genetic variants with CAD as input, the LDPred computational algorithm was implemented to derive eleven polygenic scores as previously recommended. (Vilhjálmsson, B. J. et al., 2015) These scores varied in the fraction of variants assumed to be causal for CAD. The relationship of each of the eleven polygenic scores with CAD was next assessed in the UK Biobank Phase I testing dataset comprised of 4,831 individuals with CAD and 115,455 controls. (Klarin, D. et al., 2017). The score assuming a fraction of causal variants of 0.001 (i.e., 0.1% of variants) achieved the highest area under the curve of 0.64 and was used in subsequent validation datasets (FIG. 25, Table 34). This achieved AUC for this score of 6.6 million variants was significantly higher than a previously implemented score (Khera, A. V. et al., 2016) containing only 50 variants that achieved genome-wide levels of statistical significance in previous studies (0.64 versus 0.59; p<0.001). The odds ratio for CAD among those with high (top quintile) versus low (bottom quintile) polygenic risk was 4.09 (95% CI 3.69-4.55) with the 6.6 million variant score as compared to 2.47 (95% CI 2.24-2.72) with the 50 variant score (FIG. 25B).

### Validation of the Polygenic Score in Three Clinical Scenarios

### Early-Onset CAD; VIRGO-MESA-TAICHI Cohort

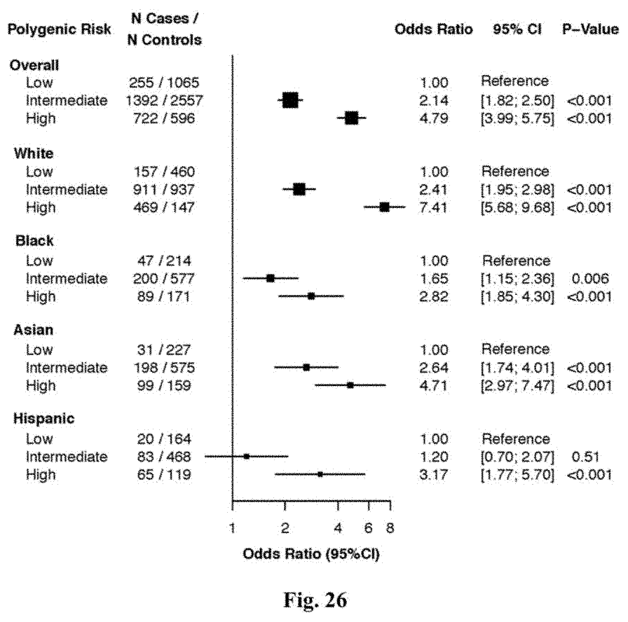

The relationship of the polygenic score to early-onset CAD was examined in the VIRGO-MESA-TAICHI case-control cohort of 6,587 individuals—2,369 cases and 4,218 controls. Mean age was 57 years and 55% of the participants were female. This multiethnic population included 3,081 (47%) white, 1,298 black (20%), 1,289 Asian (20%) and 919 (14%) Hispanic participants (eTables 2-3). As compared to those with low polygenic risk, an increased odds of early-onset CAD was noted for both the intermediate (odds ratio 2.14; 95% CI 1.82-2.50) and high (odds ratio 4.79; 95% CI 3.99-5.75) risk categories (FIG. 26).

The generalizability of the polygenic score was assessed by testing the association of polygenic risk categories with myocardial infarction in racial subpopulations. Although the score was associated with increased odds of early-onset CAD within each race (p<0.001 for each), the association was strongest in white participants (odds ratio for extreme quintiles 7.41; 95% CI 5.68-9.68) as compared with odds ratio for extreme quintiles of 2.82, 4.71, and 3.17 for Black, Asian, and Hispanic participants respectively (FIG. 26); p-value for heterogeneity by race <0.001.

### Prevalent and Incident CAD in Middle-Aged European Cohort—UK Biobank Phase II

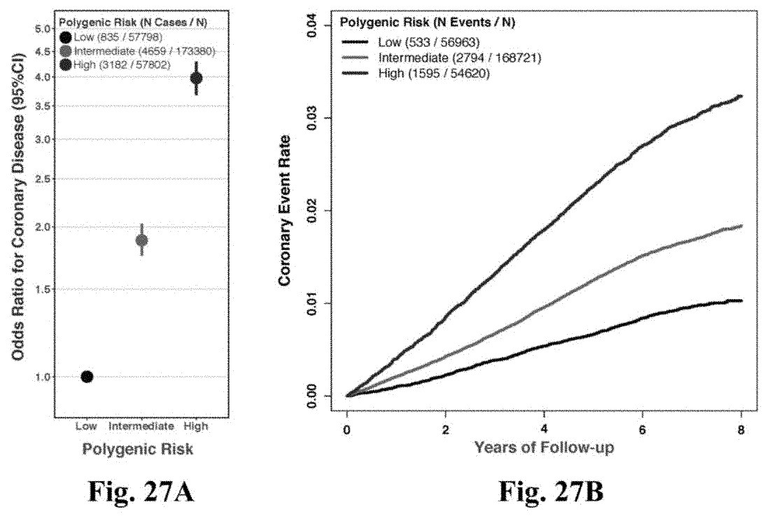

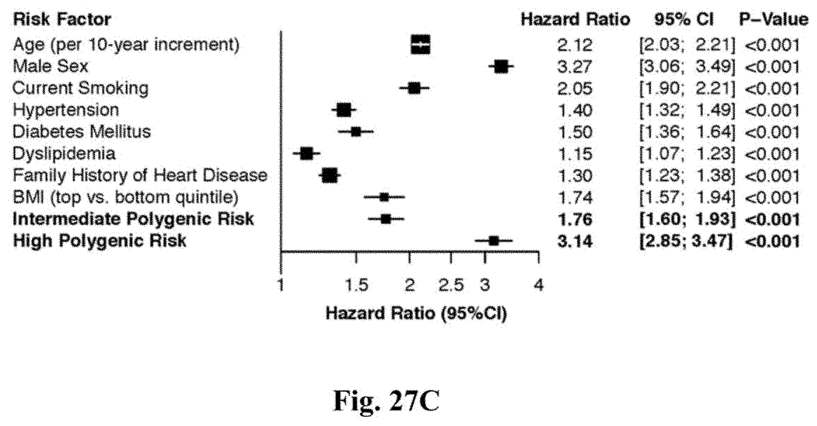

The association of the polygenic score with prevalent CAD in a middle-aged European cohort was assessed in the UK Biobank Phase II dataset (N=288,980), inclusive of 8,676 individuals with CAD and 280,304 controls (Table 37). Mean age was 57 years and 55% of the cohort was female Consistent with the observations noted in the testing dataset, an increased odds of CAD was noted for both the intermediate (odds ratio 1.88; 95% CI 1.75-2.03) and high (odds ratio 3.98; 95% CI 3.68-4.30) risk groups (FIG. 27A).

Among the 280,304 individuals free of CAD at baseline, 4,922 incident coronary events were observed over a median follow-up of 7.0 years (Table 38). Incident event rates were 1.3 (95% CI 1.2-1.5), 2.4 (2.3-2.5), and 4.3 (4.0-4.5) per 1000 person-years for individuals in the low, intermediate, and high polygenic risk categories (FIG. 27B). Compared with those in the low polygenic risk group, absolute event rates were 1.0 (95% CI 0.9-1.2; p<0.001) per 1000 person-years higher in those with intermediate risk and 2.9 (95% CI 2.7-3.1; p<0.001) higher in those with high risk. These absolute differences corresponded to hazard ratios of 1.81 (95% CI 1.65-1.99) and 3.36 (95% CI 3.04-3.77) for those with intermediate and high polygenic risk respectively in a Cox survival model with the low polygenic risk group serving as the reference group and including age, sex, and principal components of ancestry as covariates. Traditional risk factor burden tended to be higher in those with high versus low polygenic risk (Table 38). However, effect estimate attenuation was modest in a multivariable model that additionally included traditional cardiovascular risk factors—hypertension, diabetes, current smoking, dyslipidemia, family history of heart disease, and body-mass index (FIG. 27C).

TABLE 38
Baseline Characteristics of the UK Biobank Phase II Incident Events Cohort

| | | | Intermediate | | |
|---|---|---|---|---|---|
| | Overall Cohort | Low Risk | Risk | High Risk | |
| | N = 280,304 | N = 56,963 | N = 168,721 | N = 54,620 | P-value |
| Age, years | 56.75 | 56.90 | 56.75 | 56.57 | |
| | (8.03) | (8.03) | (8.03) | (8.02) | <0.001 |
| Male Gender | 124130 | 25587 | 74952 | 23591 | |
| | (44.3) | (44.9) | (44.4) | (43.2) | <0.001 |
| Hypertension | 75758 | 13763 | 45667 | 16328 | |
| | (27.0) | (24.2) | (27.1) | (29.9) | <0.001 |
| Diabetes Mellitus | 12406 | 2279 | 7475 | 2652 | |
| | (4.4) | (4.0) | (4.4) | (4.9) | <0.001 |
| Dyslipidemia | 34000 | 5438 | 20293 | 8269 | |
| | (12.1) | (9.5) | (12.0) | (15.1) | <0.001 |
| Current Smoking | 25520 | 5071 | 15266 | 5183 | 0.001 |
| | (9.1) | (8.9) | (9.1) | (9.5) | |
| FH of Heart Disease | 100036 | 17836 | 59813 | 22387 | |
| | (35.7) | (31.3) | (35.5) | (41.0) | <0.001 |
| Body-mass Index, kg/m² | 27.30 | 27.15 | 27.31 | 27.46 | |
| | (4.72) | (4.65) | (4.71) | (4.80) | <0.001 |
| Lipid-lowering | 41788 | 6748 | 25082 | 9958 | |
| Medication | (15.0) | (11.9) | (15.0) | (18.3) | <0.001 |

Values represent N (% with nonmissing values), mean (SD), or median (IQR). P-values computed via ANOVA for continuous variables (TG modeled using Kruskal-Wallis test) and chi-square test for categorical variables.
FH (family history).

Addition of the polygenic score to a baseline model containing age, sex, and principal components of ancestry led to an improvement in discrimination, increase in C-statistic from 0.733 to 0.759 (p<0.001) and reclassification, net reclassification index of 0.36 (95% CI 0.33-0.38; p<0.001). When the baseline model additionally included the traditional cardiovascular risk factors of hypertension, diabetes, current smoking, family history of heart disease, and body-mass index, addition of the polygenic score led to an increase in the C-statistic from 0.762 to 0.783 (p<0.001) and net reclassification index of 0.33 (95% CI 0.31-0.36); p<0.001.

An individual who is an extreme outlier in the polygenic score distribution may have a risk for CAD at least as great as a carrier of a familial hypercholesterolemia mutation (present in 0.5% of the population). Applicants compared the risk for CAD for those in the top 0.5% of the polygenic score distribution to the remaining 99.5% of the population, noting a substantially increased odds for prevalent CAD (odds ratio 4.46; 95% CI 3.79-5.22) and risk for incident CAD (hazard ratio 3.63; 95% CI 2.87-4.60).

An interaction of the polygenic score with age at baseline was noted (p-interaction <0.001), such that the risk gradient was more pronounced among younger individuals. For example, the hazard ratio for extreme quintiles of the polygenic score was 5.16 (3.45-7.74) among individuals <50 years of age, 4.02 (95% CI 3.28-4.92) in those 50 to <60 years, and 2.99 (95% CI 2.66-3.36) among those ≥60 years (Table 39). By contrast, no such interaction was observed based on sex (p=0.66), family history of heart disease (p=0.55), or other cardiovascular risk factors (p>0.05 for each).

TABLE 39
Association of the Polygenic Score with Incident Coronary Events according to Age

| Polygenic Risk | | | | | Incidence |
|---|---|---|---|---|---|
| Category | N Events/N | Hazard Ratio | 95% CI | P-Value | Rate^{a} |
| Age <50 years | 348/62,966 | | | | |
| Low | 28/12,519 | Reference | — | — | 0.3 |
| Intermediate | 176/37,829 | 2.10 | 1.41-3.12 | <0.001 | 0.7 |
| High | 144/12,618 | 5.16 | 3.45-7.74 | <0.001 | 1.6 |
| Age 50-60 years | 1,244/92,651 | | | | |
| Low | 119/18568 | Reference | — | — | 0.9 |
| Intermediate | 673/55,788 | 1.91 | 1.57-2.32 | <0.001 | 1.3 |
| High | 452/18,295 | 4.02 | 3.28-4.92 | <0.001 | 3.6 |
| Age ≥60 years | 3,330/124,687 | | | | |
| Low | 386/25,876 | Reference | — | — | 2.2 |
| Intermediate | 1945 75,104 | 1.77 | 1.58-1.97 | <0.001 | 3.8 |
| High | 999/23,707 | 2.99 | 2.66-3.36 | <0.001 | 6.3 |

Hazard ratios calculated using Cox regressions models with adjustment for age, sex, the first four principal components of ancestry, and a dummy variable for genotyping array used. Individuals with low polygenic risk served as the reference group.
^{a}Incidence rates are calculated per 1000 person-years of follow-up

### Incident CAD in a Middle-Aged United States Cohort—Atherosclerosis Risk in Communities

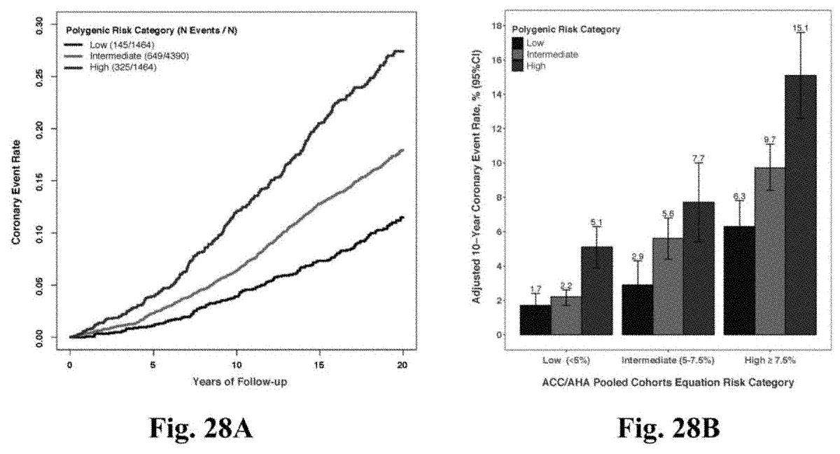

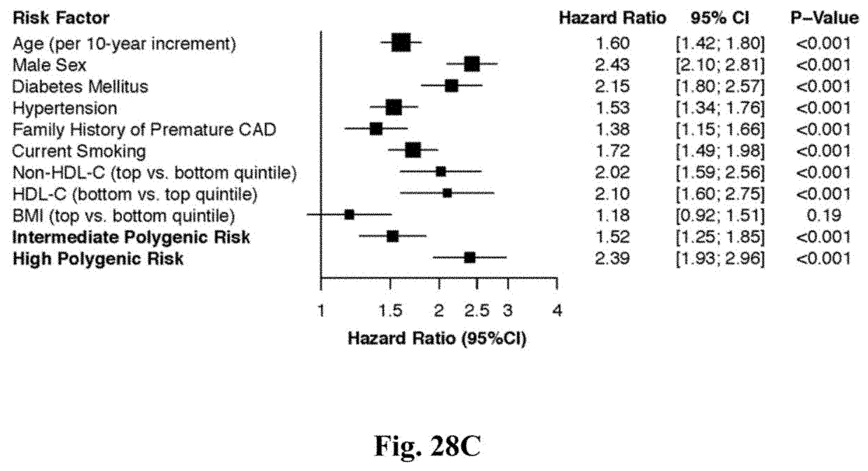

Additional validation of the association between the polygenic score and incident coronary events was provided in the ARIC prospective cohort—1,119 incident coronary events were observed in 7,318 white individuals over a median follow-up of 18.9 years. Mean age was 54 years and 54% of the participants were female (Table 40). Incident event rates were 5.6 (95% CI 4.7-6.5), 8.7 (95% CI 8.0-9.3), and 13.5 (95% CI 12.1-15.0) per 1000-person years for individuals in the low, intermediate, and high polygenic risk categories respectively (FIG. 28A). Compared with those in the low polygenic risk group, absolute event rates were 3.1 (95% CI 2.0-4.2; p<0.001) per 1000 person-years higher in those with intermediate risk and 8.0 (95% CI 6.2-9.7; p<0.001) higher in those with high risk. These absolute differences corresponded to hazard ratios of 1.62 (95% CI 1.35-1.94) and 2.78 (95% CI 2.29-3.39) for those with intermediate and high polygenic risk respectively in a Cox survival model with the low polygenic risk group serving as the reference group and including age, sex, and principal components of ancestry as covariates.

TABLE 40
Baseline Characteristics of the Atherosclerosis Risk in Communities Incident Events Cohort

| | Overall | | | | Intermediate | | | | |
|---|---|---|---|---|---|---|---|---|---|
| | Cohort | | Low Risk | | Risk | | High Risk | | |
| | N = 7,318 | | N = 1,464 | | N = 4,390 | | N = 1,464 | | P-value |
| Age, years | 54 | (5.7) | 54 | (5.8) | 54 | (5.7) | 54 | (5.7) | 0.003 |
| Male Gender | 3,330 | (46%) | 660 | (45%) | 2,025 | (46%) | 645 | (44) | 0.36 |
| Hypertension | 1,885 | (26%) | 315 | (22%) | 1,161 | (27%) | 409 | (28%) | <0.001 |
| Diabetes Mellitus | 580 | (8%) | 102 | (7%) | 346 | (8%) | 132 | (9%) | 0.12 |
| Current Smoking | 1,801 | (25%) | 356 | (24%) | 1,056 | (24%) | 389 | (27%) | 0.15 |
| FH of Premature CAD | 697 | (11%) | 103 | (8%) | 403 | (11%) | 191 | (15%) | <0.001 |
| Body-mass Index, kg/m² | 27 | (4.8) | 27 | (4.5) | 27 | (4.8) | 27 | (4.8) | 0.92 |
| Lipid Levels | | | | | | | | | |
| Total Cholesterol, | 214 | (41) | 209 | (39) | 214 | (41) | 220 | (40) | <0.001 |
| mg/dl | | | | | | | | | |
| LDL Cholesterol, | 137 | (38) | 132 | (37) | 136 | (38) | 142 | (47) | <0.001 |
| mg/dl | | | | | | | | | |
| HDL Cholesterol, | 37 | (11) | 38 | (11) | 37 | (11) | 37 | (11) | <0.001 |
| mg/dl | | | | | | | | | |
| Triglycerides, mg/dl | 113 | (81-161) | 108 | (78-156) | 113 | (81-161) | 118 | (85-166) | <0.001 |
| Statin Medication | 40 | (0.5%) | 8 | (0.6%) | 21 | (0.5%) | 11 | (0.8%) | 0.47 |

Values represent N (% with nonmissing values), mean (SD), or median (IQR). P-values computed via ANOVA for continuous variables (TG modeled using Kruskal-Wallis test) and chi-square test for categorical variables.
FH (family history); CAD (coronary artery disease). Family history of premature coronary artery disease refers to self-reported parental history of myocardial infarction prior to age 60 years.

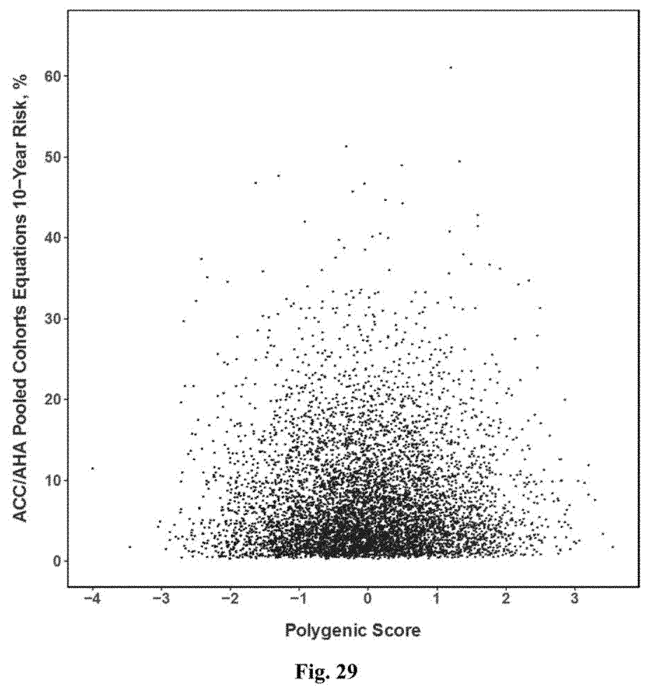

Minimal correlation between the polygenic score and predicted 10-year risk of atherosclerotic cardiovascular disease, as assessed by the ACC/AHA Pooled Cohorts Equations (see Goff, D. C. et al., Circulation. 129(25 Suppl 2), S49-73 (2014)), was observed (Spearman r=0.03; p=0.004; FIG. 29). Mean (SD) values of 7.0% (6.6), 7.3% (6.5), and 7.5% (6.8) were observed for low, intermediate, and high polygenic risk categories respectively. Consistent with the polygenic score as a largely orthogonal metric of risk, additional adjustment for the 10-year predicted risk, led to minimal attenuation of risk estimates—hazard ratios of 1.60 (95% CI 1.32-1.94) and 2.70 (2.19-3.33) for intermediate and high polygenic risk groups respectively. Furthermore, polygenic risk categories remained a significant predictor of 10-year risk in subgroups of participants with low (<5%), intermediate (≥5-7.5%), and high (≥7.5%) risk predicted by the Pooled Cohorts Equations (FIG. 28B). Similarly, polygenic risk categories remained associated with incident events in a multivariable model that included traditional cardiovascular risk factors and circulating lipid levels (FIG. 28C). Effect estimates for the polygenic score were consistent across age, sex, and 10-year risk (p-interaction >0.05 for each).

In the ARIC cohort, addition of the polygenic score to a baseline model containing age, sex, and principal components of ancestry led to an increase in the C-statistic from 0.672 to 0.697 (p<0.001) and a net reclassification index of 0.34 (95% CI 0.28-0.40). When the predicted risk as assessed by the Pooled Cohorts Equations was included in the baseline model containing age, sex, and principal components of ancestry, addition of the polygenic score led to an increase in the C-statistic from 0.726 to 0.739 (p<0.001) and net reclassification index of 0.34 (95% CI 0.28-0.41; p<0.001).

### Discussion

In this study, Applicants derived a new polygenic score for CAD inclusive of 6.6 million genetic variants. This score significantly and substantially improved prediction of CAD over previously published scores that included fewer variants. Individuals with high polygenic risk (top quintile of polygenic score), as compared to those with low polygenic risk (bottom quintile of polygenic score) had increased odds of early-onset CAD (odds ratio 4.79) and prevalent CAD in a middle-aged population-based cohort (odds ratio 3.98). Furthermore, such individuals were at significantly increased risk of incident CAD in both a large European (hazard ratio 3.36) cohort and United States (hazard ratio 2.78) prospective cohort. The polygenic score risk estimates remained significant after adjustment for traditional cardiovascular risk factors and led to an improvement in model discrimination and reclassification.

These results permit several conclusions. First, a polygenic score for CAD provides a continuous and quantitative metric for CAD that stratifies the population into varying trajectories of coronary risk. This stratification remained robust to adjustment for traditional cardiovascular risk factors, including family history of CAD (a product of shared DNA and shared environment), circulating biomarkers, and predicted 10-year risk based on the ACC/AHA Pooled Cohorts Equation. A key advantage of a DNA-based predictor is that the polygenic score can be assessed from the time of birth, well before the discriminative capacity of alternate risk prediction indices such as coronary artery calcification and circulating biomarkers becomes apparent.

Second, this finding reinforces the concept that heritable risk for complex disease may be driven by rare large-effect mutations or the cumulative impact of many small-effect variants. For example, three previous studies have identified a familial hypercholesterolemia mutation in about 0.5% of the population and noted that such individuals are at increased odd for prevalent CAD compared to non-carriers (reported odds ratios of 2.6, 3.3, and 4.2 respectively). (See, Benn, M. et al., Eur Heart J., 37, 1384-94, (2016); Abul-Husn, N. S. et al. Science 354, doi: 10.1126/science.aaf7000 (2016); Khera, A. V. et al., J Am Coll Cardiol. 67, 2578-89 (2016)). Applicants demonstrate that, compared to the remaining 99.5% of the population, individuals in the top 0.5% of the polygenic score distribution have an even higher odds ratio for prevalent CAD of 4.5.

Third, new evidence from a multiethnic cohort is provided that the polygenic score can discriminate risk across racial groups. However, consistent with the derivation and validation of this and previous scores in individuals of European ancestry, score performance was best in white individuals as compared to other racial groups. Similar findings were noted in a recent analysis of polygenic scores in predicting height, schizophrenia, and type 2 diabetes. (See Martin, A. R. et al., Am J Hum Genet., 100, 635-49 (2017)). This does not suggest that genetic risk is less important in non-white individuals. Rather, large-scale efforts to refine variant risk estimates in multiethnic populations are warranted and can help ensure that such scores would not propagate health disparities if integrated into clinical practice. (See Popejoy, A. B. et al., Nature. 538(7624), 161-64 (2016).

Ascertainment of individuals at increased polygenic risk for common diseases may facilitate intensive prevention efforts via lifestyle or pharmacotherapy. Evidence derived from randomized clinical trials suggests that those with increased polygenic risk derive increased absolute and relative coronary risk reduction with statin therapy. (See, Mega, J. L., et al., Lancet 385(9984), 2264-71 (2015), Natarajan, P. et al., Circulation 135, 2091-101 (2017)). Similarly, absolute risk reductions associated with adherence to a healthy lifestyle were highest in the high polygenic risk subgroup. (Khera et al., 2016). This potential utility must be weighed against possible untoward consequences, including increased cost of care, psychological distress or discrimination following genetic risk disclosure, and a sense of fatalism in those at high risk. Additional research is thus needed prior to widespread implementation. (See Green, E. D. et al., Nature 470(7333), 204-13 (2011)).

A key strength of this study involves the use of a recently developed computational approach to derive a comprehensive polygenic score of 6.6 million genetic variants for a complex disease and application to multiple independent datasets. Importantly, none of the CAD cases from the present validation studies were used in score derivation or testing, thus avoiding inflation of test statistics.

### REFERENCES

Gertler M M, Garn S M, White P D. Young candidates for coronary heart disease. J Am Med Assoc. 1951; 147(7):621-5.
Lehrman M A, Schneider W J, Stidhof T C, Brown M S, Goldstein J L, Russell D W. Mutation in LDL receptor: Alu-Alu recombination deletes exons encoding transmembrane and cytoplasmic domains. Science. 1985; 227(4683):140-6.
Benn M, Watts G F, Tybjerg-Hansen A, Nordestgaard B G. Mutations causative of familial hypercholesterolaemia: screening of 98 098 individuals from the Copenhagen General Population Study estimated a prevalence of 1 in 217. Eur Heart J. 2016 May 1; 37(17):1384-94.
Abul-Husn N S, Manickam K, Jones L K, et al. Genetic identification of familial hypercholesterolemia within a single U.S. health care system. Science. 2016; 354(6319) doi: 10.1126/science.aaf7000.
Khera A V, Won H H, Peloso G M, Lawson K S, Bartz T M, Deng X, van Leeuwen E M, Natarajan P, Emdin C A, et al. Diagnostic Yield and Clinical Utility of Sequencing Familial Hypercholesterolemia Genes in Patients With Severe Hypercholesterolemia. J Am Coll Cardiol. 2016 Jun. 7; 67(22):2578-89.
Kathiresan S, Melander O, Anevski D, et al. Polymorphisms associated with cholesterol and risk of cardiovascular events. N Engl J Med. 2008 Mar. 20; 358(12):1240-9.
Ripatti S, Tikkanen E, Orho-Melander M, et al. A multilocus genetic risk score for coronary heart disease: case-control and prospective cohort analyses. Lancet. 2010; 376:1393-400.
Brautbar A, Pompeii L A, Dehghan A, et al. A genetic risk score based on direct associations with coronary heart disease improves coronary heart disease risk prediction in the Atherosclerosis Risk in Communities (ARIC), but not in the Rotterdam and Framingham Offspring, Studies. Atherosclerosis. 2012; 223:421-6.
Ganna A, Magnusson P K, Pedersen N L, et al. Multilocus genetic risk scores for coronary heart disease prediction. Arterioscler Thromb Vasc Biol. 2013; 33:2267-72.
Abraham G, Havulinna A S, Bhalala O G, et al. Genomic prediction of coronary heart disease. Eur Heart J. 2016; 37(43):3267-3278.
Khera A V, Emdin C A, Drake I, et al. Genetic risk, adherence to a healthy lifestyle, and coronary disease. N Engl J Med. 2016; 375(24):2349-2358.
International Schizophrenia Consortium, Purcell S M, Wray N R, et al. Common polygenic variation contributes to risk of schizophrenia and bipolar disorder. Nature. 2009; 460(7256):748-52.
Yang J, Benyamin B, McEvoy B P, et al. Common SNPs explain a large proportion of the heritability for human height. Nat Genet. 2010; 42(7):565-9.
Locke A E, Kahali B, Berndt S I, et al. Genetic studies of body mass index yield new insights for obesity biology. Nature. 2015; 518(7538):197-206.
Vilhjálmsson B J, Yang J, Finucane H K, et al. Modeling linkage disequilibrium increases accuracy of polygenic scores. Am J Hum Genet. 2015; 97(4):576-92.
CARDIoGRAMplusC4D Consortium. Large-scale association analysis identifies new risk loci for coronary artery disease. Nat. Genet. 45, 25-33 (2013).
The 1000 Genomes Project Consortium. A global reference for human genetic variation. Nature. 2015; 526(7571):68-74.
Klarin D, Zhu Q M, Emdin C A, et al. Genetic analysis in U K Biobank links insulin resistance and transendothelial migration pathways to coronary artery disease. Nat Genet. 2017 Jul. 17. doi: 10.1038/ng.3914. [Epub ahead of print]
Lichtman J H, Lorenze N P, D'Onofrio G, et al. Variation in recovery: Role of gender on outcomes of young AMI patients (VIRGO) study design. Circ Cardiovasc Qual Outcomes. 2010; 3(6):684-93.
Assimes T L, Lee I T, Juang J M, et al. Genetics of coronary artery disease in Taiwan: A cardiometabochip study by the Taichi Consortium. PLoS One. 2016; 11(3):e0138014.
Bild D E, Bluemke D A, Burke G L, et al. Multi-ethnic study of atherosclerosis: objectives and design. Am J Epidemiol. 2002; 156:871-881.
Sudlow C, Gallacher J, Allen N, et al. U. K. Biobank: an open access resource for identifying the causes of a wide range of complex diseases of middle and old age. PLoS Med 2015; 12:e1001779.
Bycroft C, Freeman C, Petkova D, et al. Genome-wide genetic data on ˜500,000 UK Biobank participants. bioRxiv; 2017, doi: doi.org/10.110/166298.
The atherosclerosis risk in communities (aric) study: Design and objectives. The ARIC Investigators. Am J Epidemiol. 1989; 129:687-702.
Price A L, Patterson N J, Plenge R M, Weinblatt M E, Shadick N A, Reich D. Principal components analysis corrects for stratification in genome-wide association studies. Nat Genet. 2006; 38(8):904-9.
Pencina M J, D'Agostino R B Sr, D'Agostino R B Jr, Vasan R S. Evaluating the added predictive ability of a new marker: from area under the ROC curve to reclassification and beyond. Stat Med. 2008; 27(2):157-72.
Goff D C Jr, Lloyd-Jones D M, Bennett G, et al. 2013 ACC/AHA guideline on the assessment of cardiovascular risk: a report of the American College of Cardiology/American Heart Association Task Force on Practice Guidelines. Circulation. 2014; 129(25 Suppl 2):S49-73.
Martin A R, Gignoux C R, Walters R K, et al. Human demographic history impacts genetic risk prediction across diverse populations. Am J Hum Genet. 2017; 100(4):635-649.
Popejoy A B, Fullerton S M. Genomics is failing on diversity. Nature. 2016 Oct. 13; 538(7624):161-164.
Mega J L, Stitziel N O, Smith J G, et al. Genetic risk, coronary heart disease events, and the clinical benefit of statin therapy: an analysis of primary and secondary prevention trials. Lancet. 2015; 385(9984):2264-71.
Natarajan P, Young R, Stitziel N O, et al. Polygenic score identifies subgroup with higher burden of atherosclerosis and greater relative benefit from statin therapy in the primary prevention setting. Circulation. 2017; 135(22):2091-2101.
Green E D, Guyer M S; National Human Genome Research Institute. Charting a course for genomic medicine from base pairs to bedside. Nature. 2011; 470(7333):204-13.
Fry A, Littlejohns T J, Sudlow C, et al. Comparison of sociodemographic and health-related characteristics of UK Biobank participants with the general population. Am J Epidemiol. 2017 Jun. 21. doi: 10.1093/aje/kwx246. [Epub ahead of print]
CARDIoGRAMplusC4D Consortium. A comprehensive 1000 Genomes-based genome-wide association meta-analysis of coronary artery disease. Nat Genet 2015; 47:1121-1130.
Visscher P M, Hill W G, Wray N R. Heritability in the genomics era—concepts and misconceptions. Nat Rev Genet. 2008; 9(4):255-66.
Chang C C, Chow C C, Tellier L C A M, Vattikuti S, Purcell S M, Lee J J. Second-generation PLINK: rising to the challenge of larger and richer datasets. GigaScience. 2015; 4.
Tsai, M Y, Johnson C, Kao W H, et al. Cholesteryl ester transfer protein genetic polymorphisms, HDL cholesterol, and subclinical cardiovascular disease in the Multi-Ethnic Study of Atherosclerosis. Atherosclerosis. 2008; 200: 359-367.
Friedewald W T, Levy R I, Fredrickson D S. Estimation of the concentration of low-density lipoprotein cholesterol in plasma, without use of the preparative ultracentrifuge. Clin Chem. 1972; 18(6):499-502.
Hoggart C J, Parra E J, Shriver M D, Bonilla C, Kittles R A, Clayton D F, McKeigue P M. Control of confounding of genetic associations in stratified populations. Am J Hum Genet. 2003; 72(6):1492-1504.
Fix E, Hodges J L. Discriminatory analysis: Non-parametric discrimination: Consistency properties. Texas: USAF School of Aviation Medicine. 1951; pp 261-279.
Cover T, Hart P. Nearest neighbor pattern classification. IEEE Trans Inf Theory. 1967; 13:21-27.
Pedregosa F, Varoquaux G, Gramfort A, et al. Scikit-learn: Machine Learning in Python. Journal of Machine Learning Research. 2011; 12:2825-2830
Schnier C, Sudlow C, & UK Biobank Cardiac Outcomes Adjudication Group.
Definitions of acute myocardial infarction (MI) and main MI pathological types for UK Biobank phase 1 outcomes adjudication; Version 1, January 2017. Available at: biobank.ctsu.ox.ac.uk/crystal/refer.cgi?id=461.
Brown S A, Hutchinson R, Morrisett J, et al. Plasma lipid, lipoprotein cholesterol, and apoprotein distributions in selected US communities: the Atherosclerosis Risk in Communities (ARIC) Study. Arterioscler Thromb 1993; 13:1139-1158
Das S, Forer L, Schonherr S, et al. Next-generation genotype imputation service and methods. Nat Genet. 2016; 48(10):1284-1287.
Loh P R, Danecek P, Palamara P F, et al. Reference-based phasing using the Haplotype Reference Consortium panel. Nat Genet. 2016; 48(11):1443-1448.
McCarthy S, Das S, Kretzschmar W, et al. A reference panel of 64,976 haplotypes for genotype imputation. Nat Genet. 2016; 48(10):1279-83.
ARIC manual of operations. No. 2. Cohort component procedures. Chapel Hill: University of North Carolina, ARIC Coordinating Center, School of Public Health, 1987.
Howie B N, Donnelly P, Marchini J. A flexible and accurate genotype imputation method for the next generation of genome-wide association studies. PLoS Genet. 2009; 5(6):e1000529.

### Example 5

The identification of individuals at increased genetic risk for a common, complex disease can facilitate treatment or enhanced screening strategies to prevent disease manifestation. For example, with respect to coronary disease, ˜1:250 individuals carry a rare, large-effect genetic mutation causal for increased low-density lipoprotein cholesterol (N. S. Abul-Husn, et al. Genetic identification of familial hypercholesterolemia within a single U.S. health care system. Science. 354 (2016); A. V. Khera, et al. Diagnostic yield and clinical utility of sequencing familial hypercholesterolemia genes in patients with severe hypercholesterolemia. J Am Coll Cardiol. 67, 2578-2589 (2016); M. Benn, et al. Mutations causative of familial hypercholesterolaemia: screening of 98 098 individuals from the Copenhagen General Population Study estimated a prevalence of 1 in 217. Eur Heart J. 37, 1384-1394 (2016)). A recent analysis in a large U.S. health care system demonstrated that such individuals have an odds ratio for coronary disease of 2.6 when compared to non-carriers and an odds ratio of 3.7 for early-onset disease (N. S. Abul-Husn, et al. Genetic identification of familial hypercholesterolemia within a single U.S. health care system. Science. 354 (2016)). Aggressive treatment to reduce circulating low-density lipoprotein cholesterol levels among carriers of such mutations can reduce coronary disease risk (Nordestgaard B G, et al. Familial hypercholesterolaemia is underdiagnosed and undertreated in the general population: guidance for clinicians to prevent coronary heart disease: consensus statement of the European Atherosclerosis Society. Eur Heart J. 34, 3478-90a (2013)).

Beyond rare monogenic mutations, a decade of genome-wide association studies (GWAS) has demonstrated that common single nucleotide polymorphisms contribute to a range of complex diseases (P. M. Visscher, et al. 10 Years of GWAS discovery: biology, function, and translation. Am J Hum Genet. 101, 5-22 (2017)). However, because the effect size of such polymorphisms tends to be modest, any individual polymorphism has limited utility for risk prediction. Polygenic scores (PS) provide a mechanism for aggregating the cumulative impact of common polymorphisms by summing the number of risk variant alleles in each individual weighted by the impact of each allele on risk of disease (International Schizophrenia Consortium, et al. Common polygenic variation contributes to risk of schizophrenia and bipolar disorder. Nature. 460, 748-752 (2009)). Applicants recently demonstrated that a coronary disease PS consisting of 50 common variants that had achieved genome-wide levels of statistical significance in previous studies can stratify the population into varying trajectories of risk (H. Tada, et al. Risk prediction by genetic risk scores for coronary heart disease is independent of self-reported family history. Eur Heart J. 37, 561-567 (2016); A. V. Khera, et al. Genetic risk, adherence to a healthy lifestyle, and coronary disease. N Engl J Med. 375, 2349-2358 (2016)).

Simulated analyses based on GWAS effect size distributions suggest that the predictive power of such PSs may be markedly improved by considering a genome-wide set of common polymorphisms (N. Chatterjee, et al. Projecting the performance of risk prediction based on polygenic analyses of genome-wide association studies. Nat Genet. 45, 400-405 (2013); F. Dudbridge. Power and predictive accuracy of polygenic risk scores. PLoS Genet. 9, e1003348 (2013); Zhang, et al. doi.org/10.1101/175406 (2017)). But, it remains uncertain whether the extreme of a PS distribution can confer risk equivalent to a monogenic mutation (e.g., 4-fold increased risk). Here, Applicants demonstrate that a PS comprised of a genome-wide set of common variants permits identification of individuals with 4-fold increased risk for coronary disease and subsequently generalize this approach to two additional complex diseases, breast cancer and severe obesity.

In order to develop an optimized polygenic score for coronary disease, Applicants derived two new PSs and compared them with two previously published scores in a testing dataset of 120,286 individuals of European ancestry from the UK Biobank—4,831 with coronary disease and 115,455 controls (H. Tada, et al. Risk prediction by genetic risk scores for coronary heart disease is independent of self-reported family history. Eur Heart J. 37, 561-567 (2016); G. Abraham, et al. Genomic prediction of coronary heart disease. Eur Heart J. 37, 3267-3278 (2016); D. Klarin, et al. Genetic analysis in UK Biobank links insulin resistance and transendothelial migration pathways to coronary artery disease. Nat Genet. 49, 1392-1397 (2017)). The UK Biobank is a large observational study that enrolled individuals aged 45 to 69 years of age from across the United Kingdom beginning in 2006 (C. Sudlow, et al. UK biobank: an open access resource for identifying the causes of a wide range of complex diseases of middle and old age. PLoS Med. 12, e1001779 (2015)).

Applicants derived the two new PSs using summary association statistics from our earlier GWAS as a starting point for the relationship of millions of common polymorphisms to risk for coronary disease (Supp. Methods; M. Nikpay, et al. A comprehensive 1,000 Genomes-based genome-wide association meta-analysis of coronary artery disease. Nat Genet. 47, 1121-1130 (2015)). A reference population of 503 Europeans from the 1000 Genomes study was used to assess the correlation of a given polymorphism with others nearby (‘linkage disequlibrium’) (The 1000 Genomes Project Consortium. A global reference for human genetic variation. Nature. 526, 68-74 (2015)). For the first score, Applicants implemented a ‘pruning and thresholding’ strategy (PS_{P&T}) to combine independent variants (r²<0.8 with other nearby variants) that exceeded nominal significance (p-value <0.05) in the previous GWAS. For the second score, Applicants used the recently developed LDPred computational algorithm (B. J. Vilhjálmsson, et al. Modeling linkage disequilibrium increases accuracy of polygenic scores. Am J Hum Genet. 97, 576-592 (2015)). This involves a Bayesian approach to calculate a posterior mean effect for all variants based on a prior (effect size in the prior GWAS) and subsequent shrinkage based on linkage disequilibrium.

All four scores demonstrated robust association with coronary disease in the testing dataset. But, the newly-derived genome-wide polygenic score of 6.6 million common single nucleotide polymorphisms (PS_{GW}) demonstrated the maximal area-under-the-curve of 0.64 and was selected for use in subsequent analyses (Table 41).

Next, Applicants sought to validate this score in an independent dataset of the remaining 288,890 individuals of European ancestry in the UK Biobank. Mean age was 57 years and 55% of the cohort was female. 8676 (3.0%) of the participants had been diagnosed with coronary disease, as defined based on verbal interview with a trained nurse or hospitalization for myocardial infarction or coronary revascularization in the electronic health record prior to enrollment.

TABLE 41
Association of 4 polygenic scores with coronary disease in testing dataset of 120,286 individuals.
Area-under-the curve and odds ratios determined via logistic regression adjusting for the first
four principal components of ancestry. GWAS = genome-wide association study;
SD = standard deviation; P & T = pruning and thresholding; GW = genome-wide.

| | | | Area-under | Odds ratio |
|---|---|---|---|---|
| Polygenic score | Derivation strategy | N Variants | the curve | (per SD increment) |
| Tada et al. (7) | Variants that had | 50 | 0.59 | 1.38 |
| | achieved genome- | | | |
| | wide levels of | | | |
| | statistical | | | |
| | significance in prior | | | |
| | GWAS (p < 5 × 10⁻⁸) | | | |
| Abraham et al. (8) | Linkage- | 49,310 | 0.59 | 1.38 |
| | disequilibrium based | | | |
| | thinning of variants | | | |
| | from prior GWAS | | | |
| PS_{P&T} | Pruning based on | 116,859 | 0.62 | 1.54 |
| | statistical | | | |
| | significance (p < 0.05) | | | |
| | and linkage | | | |
| | disequilibrium (r² < 0.8) | | | |
| | of variants from | | | |
| | prior GWAS | | | |
| PS_{GW} | LDP red | 6,630,150 | 0.64 | 1.67 |
| | computational | | | |
| | algorithm to assign | | | |
| | weights to all | | | |
| | available variants | | | |
| | from prior GWAS | | | |
| | via explicit | | | |
| | modeling of linkage | | | |
| | disequilibrium | | | |

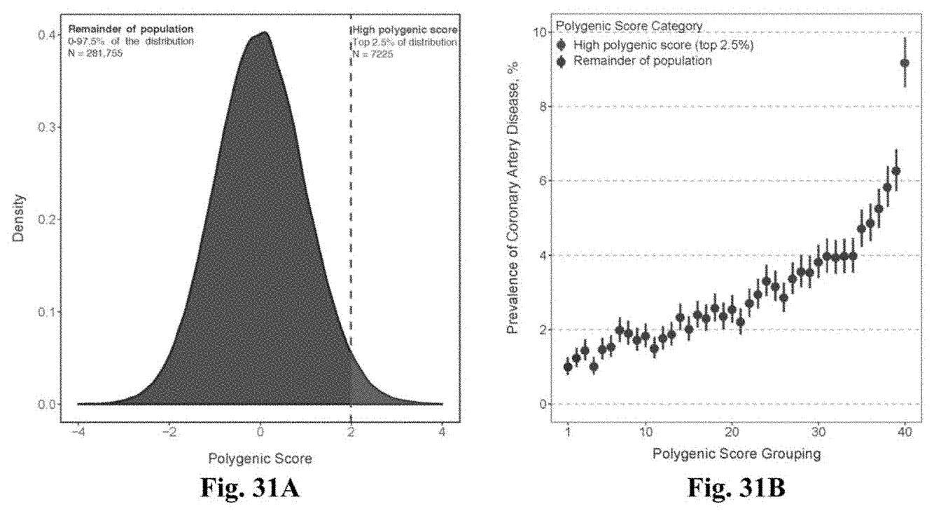

Applicants tested the hypothesis that individuals with high PS_{GW} might have risk equivalent to a monogenic coronary disease mutation (e.g., four-fold increased risk) by assessing progressively more extreme tails of the PS_{GW} distribution and comparing risk with the remainder of the population (Table 42; FIG. 31A). Across UK Biobank participants, PS_{GW} conformed to a normal distribution and individuals in the top 2.5% of the PS_{GW} distribution had a four-fold increased coronary disease risk (odds ratio 3.96) when compared with the remaining 97.5% of the population in a logistic regression model adjusted for age, sex, genotyping array, and the first four principal components of ancestry. Applicants defined those individuals in the top 2.5% of the distribution as having high PS_{GW} in subsequent analyses.

TABLE 42
Prevalence and clinical impact of high polygenic score for coronary artery disease.
Odds ratio for coronary disease calculated by comparing those with high polygenic score
to the remainder of the population in a logistic regression model adjusted for age, sex,
genotyping array, and the first four principal components of ancestry.

| | | Odds ratio | | |
|---|---|---|---|---|
| High polygenic | | for coronary | 95% Confidence | |
| score definition | Reference group | disease | interval | P-value |
| Top 20% of | Remaining 80% | 2.53 | 2.42-2.65 | <1 × 10⁻³⁰⁰ |
| distribution | | | | |
| Top 10% of | Remaining 90% | 2.89 | 2.73-3.05 | <1 × 10⁻³⁰⁰ |
| distribution | | | | |
| Top 5% of | Remaining 95% | 3.32 | 3.10-3.56 | 8.4 × 10⁻²⁶¹ |
| distribution | | | | |
| Top 2.5% of | Remaining 97.5% | 3.96 | 3.62-4.31 | 9.4 × 10⁻²⁰⁹ |
| distribution | | | | |
| Top 1% of | Remaining 99% | 4.67 | 4.11-5.30 | 3.4 × 10⁻¹²⁵ |
| distribution | | | | |
| Top 0.25% of | Remaining 99.75% | 6.34 | 5.01-7.94 | 4.7 × 10⁻⁵⁶ |
| distribution | | | | |

Coronary disease was noted in 663 of 7225 (9.2%) individuals with high PS_{GW} as compared to 8013 of 281,755 (2.8%) of those in the remainder of the distribution (FIG. 31B). Of the 8676 individuals with coronary disease, 663 (7.6%) were predisposed on the basis of high PS_{GW}. Several traditional coronary disease risk factors including family history of heart disease were enriched in those with high PS_{GW} (Table 43). However, attenuation in the risk estimate for high PS_{GW} was modest after additional adjustment for history of hypertension, type 2 diabetes, hypercholesterolemia, current smoking, and family history of heart disease (adjusted odds ratio 3.15; 95% confidence interval 2.86-3.46).

TABLE 43
Baseline characteristics according to high coronary disease polygenic score status.
Values displayed are mean (standard deviation) for continuous variables and N (%) for
categorical variables.

| | Remainder of population | | High polygenic score | | |
|---|---|---|---|---|---|
| | (0-97.5% of | | (top 2.5% of | | |
| | distribution) | | distribution) | | P-value |
| Number of individuals | 281,755 | | 7225 | | |
| Age, years | 56.9 | (8.0) | 56.7 | (8.1) | 0.01 |
| Male sex | 127,894 | (45.4%) | 3189 | (44.1%) | 0.04 |
| Hypertension | 78,999 | (28.0%) | 2460 | (34.0%) | <0.001 |
| Type 2 diabetes | 13,547 | (4.8%) | 441 | (6.1%) | <0.001 |
| Hypercholesterolemia | 38,001 | (13.5%) | 1600 | (22.1%) | <0.001 |
| Current smoking | 25,908 | (9.2%) | 691 | (9.6%) | 0.29 |
| Family history of heart | 100,856 | (35.8%) | 3364 | (46.6%) | <0.001 |
| disease | | | | | |
| Body mass index, kg/m² | 27.4 | (4.7) | 27.7 | (4.8) | <0.001 |
| Systolic blood pressure, | 140 | (19.7) | 141 | (19.6) | <0.001 |
| mmHg | | | | | |
| Lipid-lowering therapy | 47,550 | (17.0%) | 1962 | (27.3%) | <0.001 |

In order to assess the generalizability of these observations, Applicants used a similar approach to construct separate PSs for two additional complex diseases with major public health implications—breast cancer and severe obesity. As for coronary disease, Applicants used summary association statistics from large prior GWASs as a starting point for the relationship of common polymorphisms to breast cancer or body-mass index (K. Michailidou, et al. Association analysis identifies 65 new breast cancer risk loci. Nature. 551, 92-94 (2017); A. E. Locke, et al. Genetic studies of body mass index yield new insights for obesity biology. Nature. 518, 197-206 (2015)).

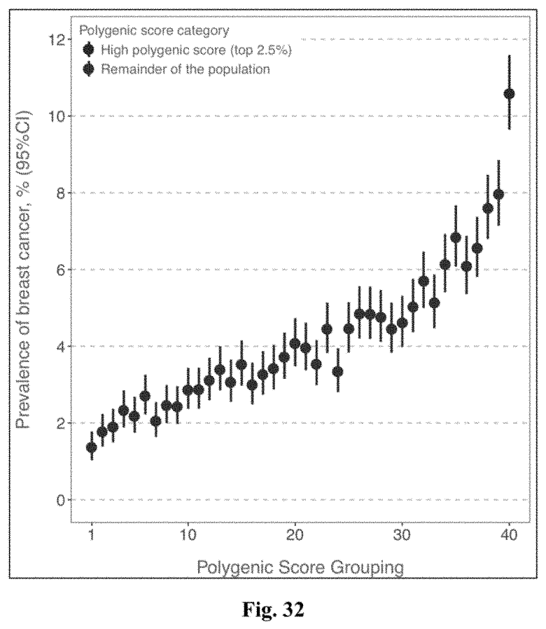

Among 157,897 females of the UK Biobank validation dataset, 6567 (4.2%) had been diagnosed with breast cancer at the time of enrollment. Individuals with high PS for breast cancer had a 2.9-fold increased risk when compared with the remaining 97.5% of the population (Table 44). Breast cancer was noted in 10.5% of individuals with high PS as compared to 4.0% of those in the remainder of the distribution (FIG. 32). Of individuals with breast cancer, 6.4% were predisposed on the basis of high PS. Attenuation in the risk estimate for high PS was modest after additional adjustment for family history of breast cancer, age at menarche, current smoking, body-mass index, and previous use of hormonal replacement therapy (adjusted odds ratio 2.78 95% confidence interval 2.49-3.09; Table 44).

TABLE 44
Baseline characteristics according to high breast cancer polygenic score
status. Values displayed are mean (standard deviation) for continuous variables and
N (%) for categorical variables. HRT—hormone replacement therapy.

| | Remainder of population | | High polygenic score | | |
|---|---|---|---|---|---|
| | (0-97.5% of | | (top 2.5% of | | |
| | distribution) | | distribution) | | P-value |
| Number of individuals | 153,949 | | 3948 | | |
| Age, years | 56.8 | (8.0) | 56.7 | (8.0) | 0.802 |
| Current smoking | 11,654 | (7.6%) | 320 | (8.1%) | 0.22 |
| Body mass index, kg/m² | 27.0 | (5.1) | 27.1 | (5.2) | 0.26 |
| Age at menarche | 13.0 | (1.6) | 13.0 | (1.6) | 0.80 |
| Number of live births | 1.8 | (1.2) | 1.8 | (1.2) | 0.65 |
| Age at first birth | 25.3 | (4.5) | 25.3 | (4.5) | 0.783 |
| Prior use of HRT | 60,716 | (40%) | 1,502 | (38%) | 0.076 |
| Fam. history of breast | 17,272 | (11.2%) | 668 | (16.9%) | <0.001 |
| cancer | | | | | |
| Had mammogram | 124,743 | (81%) | 3,261 | (83%) | .01 |
| screening | | | | | |

TABLE 45
Prevalence and clinical impact of high polygenic score for breast cancer
and severe obesity (body-mass index ≥40 kg/m²). Breast cancer analysis was restricted to
females. Odds ratios calculated by comparing those with high polygenic score to the remainder
of the population in a logistic regression model adjusted for age, sex (for severe obesity only),
genotyping array, and the first four principal components of ancestry.

| High polygenic | | | | 95% Confidence | |
|---|---|---|---|---|---|
| score definition | Reference group | | Odds ratio | interval | P-value |
| Breast cancer | | | | | |
| Top 20% of | Remaining | 80% | 2.19 | 2.08-2.31 | 3.6 × 10⁻¹⁸⁵ |
| distribution | | | | | |
| Top 10% of | Remaining | 90% | 2.34 | 2.19-2.49 | 1.7 × 10⁻¹⁵⁰ |
| distribution | | | | | |
| Top 5% of | Remaining | 95% | 2.57 | 2.36-2.78 | 1.3 × 10⁻¹¹⁴ |
| distribution | | | | | |
| Top 2.5% of | Remaining | 97.5% | 2.89 | 2.60-3.21 | 1.8 × 10⁻⁸⁶ |
| distribution | | | | | |
| Top 1% of | Remaining | 99% | 3.62 | 3.11-4.20 | 1.3 × 10⁻⁶³ |
| distribution | | | | | |
| Top 0.25% of | Remaining | 99.75% | 4.43 | 3.33-5.79 | 4.6 × 10⁻²⁶ |
| distribution | | | | | |
| Severe obesity | | | | | |
| Top 20% of | Remaining | 80% | 3.88 | 3.67-4.10 | <1 × 10⁻³⁰⁰ |
| distribution | | | | | |
| Top 10% of | Remaining | 90% | 4.29 | 4.05-4.55 | <1 × 10⁻³⁰⁰ |
| distribution | | | | | |
| Top 5% of | Remaining | 95% | 4.82 | 4.49-5.17 | <1 × 10⁻³⁰⁰ |
| distribution | | | | | |
| Top 2.5% of | Remaining | 97.5% | 5.54 | 5.07-6.05 | <1 × 10⁻³⁰⁰ |
| distribution | | | | | |
| Top 1% of | Remaining | 99% | 6.15 | 5.41-6.97 | 5.8 × 10⁻¹⁷⁴ |
| distribution | | | | | |
| Top 0.25% of | Remaining | 99.75% | 6.77 | 5.31-8.52 | 1.5 × 10⁻⁵⁶ |
| distribution | | | | | |

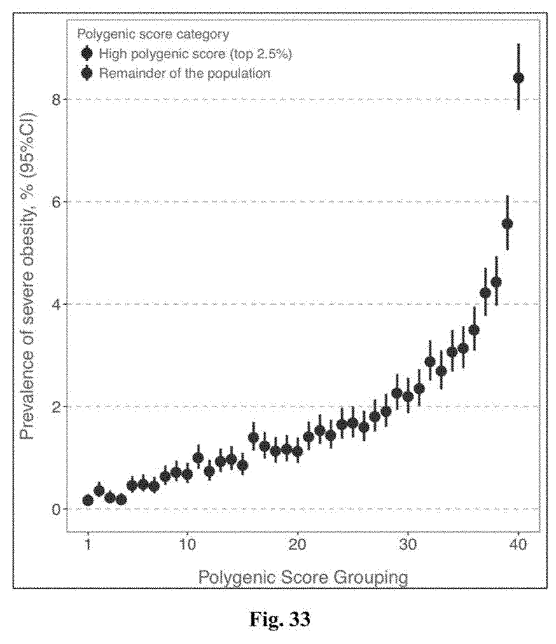

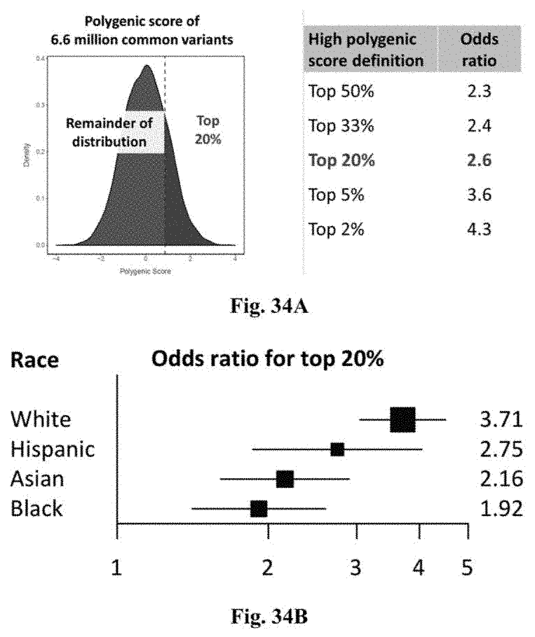

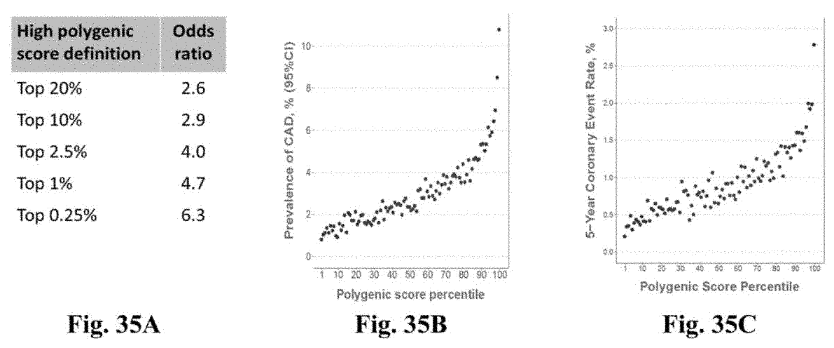

Among 288,018 individuals of the UK Biobank validation dataset with body-mass index available, 5232 (1.8%) were severely obese at the time of enrollment, defined as body-mass index ≥40 kg/m². Individuals with high PS had a 5.5-fold increased risk of severe obesity when compared with the remaining 97.5% of the population (Table 45). Severe obesity was noted in 8.4% of individuals with high body-mass index PS as compared to 1.6% of those in the remainder of the distribution (FIG. 33). Of individuals with severe obesity, 11.6% were predisposed on the basis of high PS. Results were similar when considering a less stringent definition for obesity of body-mass index ≥30 kg/m² (Table 46).

TABLE 46
Prevalence and clinical impact of high polygenic score for obesity (body-
mass index ≥ 30 kg/m²). Odds ratios calculated by comparing those with high polygenic score to
the remainder of the population in a logistic regression model adjusted for age, sex, genotyping
array, and the first four principal components of ancestry.

| High polygenic | | | | 95% Confidence | |
|---|---|---|---|---|---|
| score definition | Reference group | | Odds ratio | interval | P-value |
| Obesity | | | | | |
| Top 20% of | Remaining | 80% | 2.56 | 2.51-2.61 | <1 × 10⁻³⁰⁰ |
| distribution | | | | | |
| Top 10% of | Remaining | 90% | 2.74 | 2.68-2.81 | <1 × 10⁻³⁰⁰ |
| distribution | | | | | |
| Top 5% of | Remaining | 95% | 3.01 | 2.91-3.11 | <1 × 10⁻³⁰⁰ |
| distribution | | | | | |
| Top 2.5% of | Remaining | 97.5% | 3.42 | 3.26-3.58 | <1 × 10⁻³⁰⁰ |
| distribution | | | | | |
| Top 1% of | Remaining | 99% | 4.00 | 3.72-4.31 | 9.8 × 10⁻²⁹⁵ |
| distribution | | | | | |
| Top 0.25% of | Remaining | 99.75% | 4.47 | 2.86-5.19 | 5.0 × 10⁻⁸⁷ |
| distribution | | | | | |

For three common diseases, Applicants demonstrate that the incorporation of a genome-wide set of common polymorphisms into a PS can identify subsets of the population at substantially increased risk.

These results permit several conclusions. First, Applicants provide empiric evidence that the cumulative impact of common polymorphisms on risk of disease can approach that of rare, monogenic mutations. The predictive capacity of PSs will likely continue to improve as larger discovery GWAS studies more precisely define the effect sizes for common polymorphisms across the genome (N. Chatterjee, et al. Projecting the performance of risk prediction based on polygenic analyses of genome-wide association studies. Nat Genet. 45, 400-405 (2013); F. Dudbridge. Power and predictive accuracy of polygenic risk scores. PLoS Genet. 9, e1003348 (2013); Y. Zhang, et al. doi.org/10.1101/175406 (2017)). Second, high PS_{GW} seems operable in a much larger fraction of the population as compared to rare monogenic mutations. For coronary disease, the largest gene-sequencing study to date identified a monogenic driver mutation related to increased low-density lipoprotein cholesterol in 94 of 12,298 (0.76%) afflicted individuals (N. S. Abul-Husn, et al. Genetic identification of familial hypercholesterolemia within a single U.S. health care system. Science. 354 (2016)). Here, Applicants identify high PS_{GW} in 7.6% of individuals with coronary disease, a prevalence an order of magnitude higher. Third, traditional risk factor differences of high PS_{GW} individuals versus the remainder of the distribution are modest and these individuals would thus be difficult to identify without direct genotyping. Fourth, a key advantage of a DNA-based diagnostic such as PS_{GW} is that it can be assessed from the time of birth, well before the discriminative capacity of most traditional risk factors emerges, and may thus facilitate intensive prevention efforts. For example, Applicants recently demonstrated that high polygenic risk for coronary disease may be offset by adherence to a healthy lifestyle or cholesterol-lowering therapy with statin medications (A. V. Khera, et al. Genetic risk, adherence to a healthy lifestyle, and coronary disease. N Engl J Med. 375, 2349-2358 (2016); J. L. Mega, et al. Genetic risk, coronary heart disease events, and the clinical benefit of statin therapy: an analysis of primary and secondary prevention trials. Lancet. 385, 2264-2271 (2015); P. Natarajan, et al. Polygenic risk score identifies subgroup with higher burden of atherosclerosis and greater relative benefit from statin therapy in the primary prevention setting. Circulation. 135, 2091-2101 (2017)). Finally, Applicants demonstrate similar patterns for two additional heritable diseases—breast cancer and severe obesity—suggesting that this approach will provide a generalizable framework for risk stratification across a range of common, complex diseases.

### REFERENCES

N. S. Abul-Husn, et al. Genetic identification of familial hypercholesterolemia within a single U.S. health care system. Science. 354 (2016).
A. V. Khera, et al. Diagnostic yield and clinical utility of sequencing familial hypercholesterolemia genes in patients with severe hypercholesterolemia. J Am Coll Cardiol. 67, 2578-2589 (2016).
M. Benn, et al. Mutations causative of familial hypercholesterolaemia: screening of 98 098 individuals from the Copenhagen General Population Study estimated a prevalence of 1 in 217. Eur Heart J. 37, 1384-1394 (2016).
Nordestgaard B G, et al. Familial hypercholesterolaemia is underdiagnosed and undertreated in the general population: guidance for clinicians to prevent coronary heart disease: consensus statement of the European Atherosclerosis Society. Eur Heart J. 34, 3478-90a (2013).
P. M. Visscher, et al. 10 Years of GWAS discovery: biology, function, and translation. Am J Hum Genet. 101, 5-22 (2017).
International Schizophrenia Consortium, et al. Common polygenic variation contributes to risk of schizophrenia and bipolar disorder. Nature. 460, 748-752 (2009).
H. Tada, et al. Risk prediction by genetic risk scores for coronary heart disease is independent of self-reported family history. Eur Heart J. 37, 561-567 (2016).
A. V. Khera, et al. Genetic risk, adherence to a healthy lifestyle, and coronary disease. N Engl J Med. 375, 2349-2358 (2016).
N. Chatterjee, et al. Projecting the performance of risk prediction based on polygenic analyses of genome-wide association studies. Nat Genet. 45, 400-405 (2013).
F. Dudbridge. Power and predictive accuracy of polygenic risk scores. PLoS Genet. 9, e1003348 (2013).
Y. Zhang, et al. doi.org/10.1101/175406 (2017).
G. Abraham, et al. Genomic prediction of coronary heart disease. Eur Heart J. 37, 3267-3278 (2016).
D. Klarin, et al. Genetic analysis in UK Biobank links insulin resistance and transendothelial migration pathways to coronary artery disease. Nat Genet. 49, 1392-1397 (2017).
C. Sudlow, et al. UK biobank: an open access resource for identifying the causes of a wide range of complex diseases of middle and old age. PLoS Med. 12, e1001779 (2015).
M. Nikpay, et al. A comprehensive 1,000 Genomes-based genome-wide association meta-analysis of coronary artery disease. Nat Genet. 47, 1121-1130 (2015).
The 1000 Genomes Project Consortium. A global reference for human genetic variation. Nature. 526, 68-74 (2015).
B. J. Vilhjálmsson, et al. Modeling linkage disequilibrium increases accuracy of polygenic scores. Am J Hum Genet. 97, 576-592 (2015).
K. Michailidou, et al. Association analysis identifies 65 new breast cancer risk loci. Nature. 551, 92-94 (2017)
A. E. Locke, et al. Genetic studies of body mass index yield new insights for obesity biology. Nature. 518, 197-206 (2015).
J. L. Mega, et al. Genetic risk, coronary heart disease events, and the clinical benefit of statin therapy: an analysis of primary and secondary prevention trials. Lancet. 385, 2264-2271 (2015).

P. Natarajan, et al. Polygenic risk score identifies subgroup with higher burden of atherosclerosis and greater relative benefit from statin therapy in the primary prevention setting. Circulation. 135, 2091-2101 (2017).
P. Kühnen, et al. Proopiomelanocortin deficiency treated with a melanocortin-4 receptor agonist. N Engl J Med. 375, 240-246 (2016).
M. Lek, et al. Analysis of protein-coding genetic variation in 60,706 humans. Nature. 536, 285-91 (2016).
A. R. Martin, et al. Human demographic history impacts genetic risk prediction across diverse populations. Am J Hum Genet. 100, 635-649 (2017).
C. C. Chang, et al. Second-generation PLINK: rising to the challenge of larger and richer datasets. GigaScience. 4, 7 (2015).
C. Bycroft C, et al. Genome-wide genetic data on ˜500,000 UK Biobank participants. doi.org/10.1101/166298 (2017).

### Materials and Methods

### Testing Dataset

In order to determine which of several polygenic risk score (PS) approaches yielded the maximal coronary disease risk discrimination, Applicants applied various PS to a testing dataset from the UK Biobank (D. Klarin, et al. Genetic analysis in UK Biobank links insulin resistance and transendothelial migration pathways to coronary artery disease. Nat Genet. 49, 1392-1397 (2017)). The UK Biobank is a large prospective cohort study that enrolled individuals from across the United Kingdom, aged 40-69 years at time of recruitment, starting in 2006 (C. Sudlow, et al. UK biobank: an open access resource for identifying the causes of a wide range of complex diseases of middle and old age. PLoS Med. 12, e1001779 (2015)). Individuals underwent a series of anthropometric measurements and surveys, including medical history review with a trained nurse. The testing dataset was comprised of 120,286 individuals of European ancestry, including 4,831 participants with prevalent coronary disease and 115,455 controls.

### Coronary Disease Polygenic Score Derivation

Polygenic scores provide a quantitative metric of an individuals inherited risk based on the cumulative impact of many variants. Weights are generally assigned to each genetic variant according to the strength of their association with disease risk (effect estimate). Individuals are scored based on how many risk alleles they have for each variant (e.g. 0, 1, 2 copies) included in the polygenic score.

Applicants tested four distinct approaches to PS derivation, ultimately choosing the best score in an independent testing dataset for subsequent analysis in the validation cohort.

First, Applicants applied a previously reported PS of 50 common genetic variants that had achieved genome-wide levels of statistical significance in earlier studies (H. Tada, et al. Risk prediction by genetic risk scores for coronary heart disease is independent of self-reported family history. Eur Heart J. 37, 561-567 (2016); A. V. Khera, et al. Genetic risk, adherence to a healthy lifestyle, and coronary disease. N Engl J Med. 375, 2349-2358 (2016)). Our prior work demonstrated that this score was predictive of incident coronary disease events in prospective cohort studies of >50,000 individuals.

Second, Applicants applied a PS comprised of 49,310 genetic variants that was derived from a 2013 CARDIoGRAMplusC4D genome-wide association study (GWAS) based on the Metabochip genotyping array (G. Abraham, et al. Genomic prediction of coronary heart disease. Eur Heart J. 37, 3267-3278 (2016)). To avoid redundancy due to linkage disequilibrium (LD), the correlation in inheritance pattern of nearby variants, the reported summary association statistics were thinned based on various LD r² values. An r² value of 0.7 was determined to be the optimal threshold via empiric testing of a range of values in an independent dataset. This score was previously shown to predict incident coronary disease events in multiple distinct cohorts (G. Abraham, et al. Genomic prediction of coronary heart disease. Eur Heart J. 37, 3267-3278 (2016)).

Third, Applicants computed a new score using a p-value and LD-driven clumping procedure in PLINK version 1.90b (C. C. Chang, et al. Second-generation PLINK: rising to the challenge of larger and richer datasets. GigaScience. 4, 7 (2015)). Input included summary coronary disease association statistics for 8.3 million SNPs from the 2015 CARDIoGRAMplusC4D 1000 Genomes imputed GWAS of primarily European individuals and a reference LD panel of 503 European samples from 1000 Genomes phase 3 version 5 (M. Nikpay, et al. A comprehensive 1,000 Genomes-based genome-wide association meta-analysis of coronary artery disease. Nat Genet. 47, 1121-1130 (2015); The 1000 Genomes Project Consortium. A global reference for human genetic variation. Nature. 526, 68-74 (2015)). In brief, the algorithm forms clumps around SNPs with association p-values less than a provided threshold. Each clump contains all SNPs within 250 kb of the index SNP that are also in LD with the index SNP as determined by a provided r² threshold in the LD reference population. The algorithm iteratively cycles through all index SNPs, beginning with the smallest p-value, only allowing each SNP to appear in one clump. The final output contains the most significantly coronary disease associated SNP for each LD-based clump across the genome. A PS was built containing the index SNPs of each clump with association estimate betas (log odds) as weights. PSs were created over a range of p-value (1, 0.5, 0.05, 5×10-4, 5×10-6, 5×10-8) and r² (0.2, 0.4, 0.6, 0.8) thresholds. The best score for this approach was chosen based on maximal area-under-the curve (AUC) in the testing dataset. This score was based on a p-value for statistical significance in the original GWAS of <0.05 and r² value of <0.8.

Fourth, Applicants computed another new score using the using the recently developed LDpred computational algorithm (B. J. Vilhjálmsson, et al. Modeling linkage disequilibrium increases accuracy of polygenic scores. Am J Hum Genet. 97, 576-592 (2015)). LDpred creates a polygenic score using genome-wide variation with weights derived from a set of GWAS summary statistics. Unlike other methods that use variants most strongly associated with disease risk or a set of independent variants across the genome, LDpred includes all available variants in the derived risk score by shrinking effect estimate weights (log-odds) based on an external LD reference panel. This Bayesian approach calculates a posterior mean effect size for each variant based on a prior (association with coronary disease in the 2015 CARDIoGRAMplusC4D GWAS) and subsequent shrinkage based on the extent to which this variant is correlated with similarly associated variants in a reference population of 503 European samples from 1000 Genomes phase 3 version 5 (M. Nikpay, et al. A comprehensive 1,000 Genomes-based genome-wide association meta-analysis of coronary artery disease. Nat Genet. 47, 1121-1130 (2015); The 1000 Genomes Project Consortium. A global reference for human genetic variation. Nature. 526, 68-74 (2015)). The underlying Gaussian distribution additionally considers the fraction of causal (e.g. non-zero effect sizes) markers, referred to as ρ. Because this fraction is unknown for any given disease, a range of 7 plausible values was trialed in the testing dataset. Single nucleotide polymorphisms (SNPs) with ambiguous strand (A/T or C/G) or minor allele frequency less than 1% were removed from the score derivation. This left 6,630,150 variants available for inclusion. In accordance with recommendations from the LDpred authors, a linkage disequilibrium radius was set at 2210 variants, equivalent to the number of SNPs used as input divided by 3000. A range of p, the fraction of causal variants, was used—1, 0.3, 0.1, 0.03, 0.01, 0.003, 0.001—along with an infinitesimal (each variant assumed to contribute to disease risk) and unweighted model (raw log-odds for all variants input). The score with maximal AUC in the testing dataset (p=0.001) was carried forward in subsequent analysis.

### Polygenic Score Calculation

Scores were generated by multiplying the genotype dosage of each risk allele for each variant by its respective weight, and then summing across all variants in the score. Incorporating genotype dosages accounts for uncertainty in genotype imputation. All calculations were performed using the Hail software platform (github.com/hail-is/hail). Over 99.9% of variants in the LDpred-derived polygenic scores were available for scoring purposes in the testing dataset with sufficient imputation quality (INFO >0.3).

### Validation Cohort

The validation cohort was comprised of 288,980 UK Biobank participants distinct from those in the testing dataset described above. Individuals in the UK Biobank underwent genotyping with one of two closely related custom arrays (UK BiLEVE Axiom Array or UK Biobank Axiom Array) consisting of over 800,000 genetic markers scattered across the genome. Additional genotypes were imputed centrally using the Haplotype Reference Consortium resource as previously reported (C. Bycroft C, et al. Genome-wide genetic data on ˜500,000 UK Biobank participants. doi.org/10.1101/166298 (2017)). In order to analyze individuals with a relatively homogenous ancestry and owing to small percentages of non-British individuals, the present analysis was restricted to the white British ancestry individuals. This subpopulation was constructed centrally using a combination of self-reported ancestry and genetically confirmed ancestry using principal components. Additional exclusion criteria included outliers for heterozygosity or genotype missingness, discordant reported versus genotypic sex, putative sex chromosome aneuploidy, or withdrawal of informed consent. Each of these parameters was derived centrally as previously reported (C. Bycroft C, et al. Genome-wide genetic data on ˜500,000 UK Biobank participants. doi.org/10.1101/166298 (2017)).

The 288,980 remaining participants served as the validation dataset for the prevalent coronary disease analysis. Current smoking, lipid lowering-medication, and parental history of heart disease were determined by self-report at the time of enrollment survey. Diabetes mellitus, hypertension, and dyslipidemia were assessed based on a combination of self-report or hospitalization diagnosis code prior to date of UK Biobank enrollment reflecting these conditions.

Diagnosis of prevalent coronary disease was based on a composite of myocardial infarction or coronary revascularization. Data from hospital admissions was available via the Hospital Episode Statistics for England, Scottish Morbidity Record, and Patient Episode Database for Wales. Myocardial infarction was based on self-report or hospital admission diagnosis, as performed centrally. This included individuals with ICD-9 codes of 410.X, 411.0, 412.X, 429.79 or ICD-10 codes of I21.X, I22.X, I23.X, 124.1, 125.2 in hospitalization records.

### Assessment of Generalizability to Additional Complex Diseases

Applicants sought to generalize the approach to polygenic score derivation, testing, and validation for two additional complex traits—breast cancer and severe obesity. Polygenic scores for breast cancer were creating using the pruning and thresholding approach noted above. Input included summary association statistics from the 2017 OncoArray Consortium GWAS and a reference LD panel of 503 European samples from 1000 Genomes phase 3 version 5 (The 1000 Genomes Project Consortium. A global reference for human genetic variation. Nature. 526, 68-74 (2015); K. Michailidou, et al. Association analysis identifies 65 new breast cancer risk loci. Nature. 551, 92-94 (2017)). Owing to few male participants with breast cancer, analyses were restricted to female participants for both the testing and validation datasets. Prevalent breast cancer was based on self-report in interview with a trained nurse or a hospitalization for breast cancer prior to enrollment. The testing dataset was comprised of 63,349 individuals, of whom 2576 (4.1%) had been diagnosed with breast cancer. A PS based on variant pruning (r²<0.2) and a p-value for statistical significance in the original GWAS of <0.0005 obtained the highest AUC of 0.62 (odds ratio per standard deviation increment 1.54, 95% confidence interval 1.48-1.61) and was used in subsequent validation dataset analyses. 157,897 participants in the UK validation dataset were female (54.7%), of whom 6,567 (4.2%) had been diagnosed with breast cancer.

Polygenic scores for obesity were created using the pruning and thresholding and LDpred approaches as noted above. Input included summary association statistics from the 2015 Genome-Wide Investigation of Anthropometric Traits (GIANT) GWAS and a reference LD panel of 503 European samples from 1000 Genomes phase 3 version 5 (The 1000 Genomes Project Consortium. A global reference for human genetic variation. Nature. 526, 68-74 (2015); A. E. Locke, et al. Genetic studies of body mass index yield new insights for obesity biology. Nature. 518, 197-206 (2015)). As for coronary disease, the relationship of each score to severe obesity was determined in the testing dataset of 120,286 individuals, of whom 2,417 were diagnosed with severe obesity on the basis of body-mass index ≥40 kg/m². The best score was chosen based on maximal AUC in this testing dataset. A score of 2,100,303 variants based on the LDPred algorithm (p=0.03) obtained the highest AUC of 0.72 (odds ratio per standard deviation increment of 2.27; 95% confidence interval 2.17-2.36) and was used in the subsequent validation dataset analyses. Body-mass index was available in 288,018 of 288,980 (99.7%) of the validation dataset used for coronary disease, and these individuals served as the validation cohort for the severe obesity analysis.

### Statistical Analysis

Multiple PSs were generated using the approaches generated above and scores extracted in the UK Biobank testing dataset. The discriminative capacity of each score was tested by calculating the AUC of a logistic regression model predicting coronary disease status with additional adjustment for the first four principal components of ancestry. Odds ratio per standard deviation increment was additionally determined to facilitate comparison across scores and to previous studies.

In the validation cohort, Applicants tested the hypothesis that individuals in the extreme of the PS distribution might have a four-fold increased risk of coronary disease as compared to the remainder of the population. Starting with the top 20% of the PS distribution versus all others, Applicants tested progressively more extreme segments of the distribution until a four-fold risk increase was noted. This assessment was performed via a logistic regression model that adjusted for age, sex, genotyping array, and the first four principal components of ancestry. Baseline characteristics between those with high PS versus the remainder of the population were tabulated and tests for statistical significance compared via t-test for continuous and chi-square test for categorical variable. A second model adjusting for traditional cardiovascular risk factors—diabetes mellitus, hypertension, smoking status, hypercholesterolemia, family history of heart disease, and body mass index—was then constructed.

To assess for a gradient of risk for prevalent disease across the PS distribution, individuals were binned into groupings of 2.5% of the population and prevalence of coronary disease tabulated. Analyses for severe obesity and breast cancer were conducted in a similar fashion.

### Example 6

A key public health need is to identify individuals at high risk for a given disease to enable enhanced screening or preventive therapies. Because most common diseases have a genetic component, one important approach is to stratify individuals based on inherited DNA variation. Proposed clinical applications have largely focused on finding carriers of rare monogenic mutations at several-fold increased risk. Although most disease risk is polygenic in nature, it has not yet been possible to use polygenic predictors to identify individuals at risk comparable to monogenic mutations. This example shows exemplary methods for developing and validating genome-wide polygenic scores for five common diseases. The approach identified 8.0%, 6.1%, 3.5%, 3.2% and 1.5% of the population at greater than three-fold increased risk for coronary artery disease (CAD), atrial fibrillation, type 2 diabetes, inflammatory bowel disease, and breast cancer, respectively. For CAD, this prevalence was 20-fold higher than the carrier frequency of rare monogenic mutations conferring comparable risk.

For various common diseases, genes have been identified in which rare mutations confer several-fold increased risk in heterozygous carriers. An important example is the presence of a familial hypercholesterolemia mutation in 0.4% of the population, which confers an up to 3-fold increased risk for coronary artery disease (CAD). Aggressive treatment to lower circulating cholesterol levels among such carriers can significantly reduce risk. Another example is the p.E508K missense mutation in HNFA, with carrier frequency of 0.1% of the general population and 0.7% of Latinos,⁸ which confers up to 5-fold increased risk for type 2 diabetes. Although ascertainment of monogenic mutations can be highly relevant for carriers and their families, the vast majority of disease occurs in those without such mutations.

For most common diseases, polygenic inheritance, involving many common genetic variants of small effect, plays a greater role than rare monogenic mutations. Previous studies to create GPS had only limited success, providing insufficient risk stratification for clinical utility (for example, identifying 20% of a population at 1.4-fold increased risk relative to the rest of the population).¹² These initial efforts were hampered by three challenges: (i) the small size of initial genome-wide association studies (GWAS), which affected the precision of the estimated impact of individual variants on disease risk; (ii) limited computational methods for creating GPS; and (iii) lack of large datasets needed to validate and test GPS.

Using much larger studies and improved algorithms, this example shows that a GPS can identify subgroups of the population with risk approaching or exceeding that of a monogenic mutation. Applicant studied five common diseases with major public health impact—CAD, atrial fibrillation, type 2 diabetes, inflammatory bowel disease, and breast cancer.

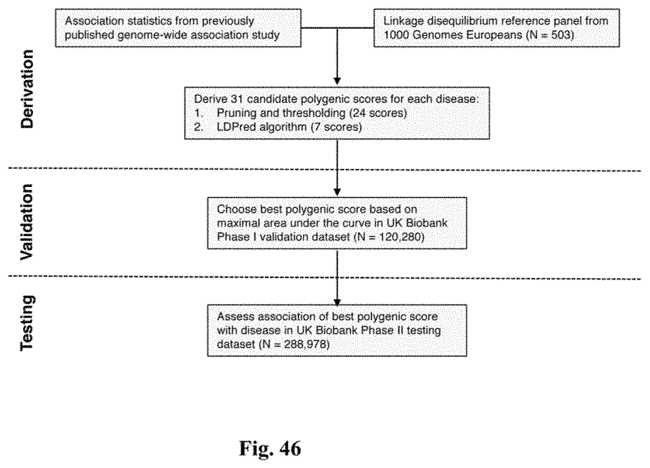

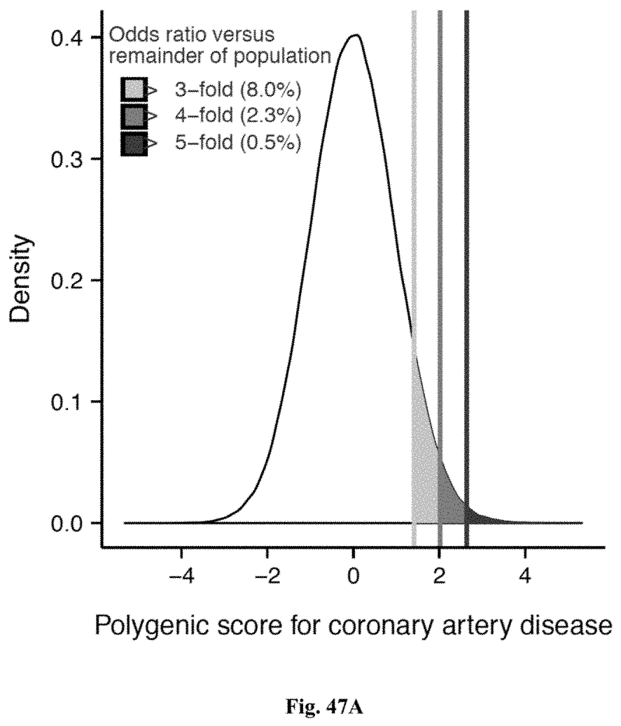

For each of the diseases, Applicant created several candidate GPS based on summary statistics and imputation from recent large GWAS in participants of primarily European ancestry (Table 47). Specifically, Applicant derived 24 predictors based on a pruning and thresholding method and 7 additional predictors using the recently described LDPred algorithm (FIG. 46; Tables 48-53). The UK Biobank has genotype data and extensive phenotypic information on 409,258 participants of British ancestry (average age 57 years; 55% female).

TABLE 47
Genome-wide polygenic score derivation and testing for five common, complex diseases. GWAS - genome-
wide association study; AUC - area under the receiver-operator curve; GPS - genome-wide polygenic
score AUC was determined using a logistic regression model adjusted for age, sex, genotyping array,
the first four principal components of ancestry. Breast cancer analysis was restricted to female participants.
For the LDPred algorithm, the tuning parameter ρ reflects the proportion of polymorphisms assumed
to be causal for the disease. For the pruning and thresholding strategy, r² reflects degree of
independence from other variants in the linkage disequilibrium reference panel and p reflects the
p-value noted for a given variant in the discovery GWAS.

| | | Prevalence | | | | AUC | AUC |
|---|---|---|---|---|---|---|---|
| | N in | in | Prevalence | | | (95% CI) in | (95% CI) in |
| | discovery | validation | in testing | Polymorphisms | Tuning | validation | testing |
| Disease | GWAS^{Refereuce} | dataset | dataset | in GPS | parameter | dataset | dataset |
| Coronary | 60,801 cases/ | 3,963/ | 8,676/ | 6,630,150 | LDPred (p = | 0.81 | 0.81 |
| artery disease | 123,504 | 120,280 | 288,978 | | 0.001) | (0.80- | (0.81- |
| | controls¹⁶ | (3.4%) | (3.0%) | | | 0.81) | 0.81) |
| Atrial | 17,931 cases/ | 2,024/ | 4,576/ | 6,730,541 | LDPred (p = | 0.77 | 0.77 |
| fibrillation | 115,142 | 120,280 | 288,978 | | 0.003) | (0.76- | (0.76- |
| | controls³⁰ | (1.7%) | (1.6%) | | | 0.78) | 0.77) |
| Type 2 | 26,676 cases/ | 2,785/ | 5,853/ | 6,917,436 | LDPred (p = | 0.72 | 0.73 |
| diabetes | 132,532 | 120,280 | 288,978 | | 0.01) | (0.72- | (0.72- |
| | controls³¹ | (2.4%) | (2.0%) | | | 0.73) | 0.73) |
| Inflammatory | 12,882 cases/ | 1,360/ | 3,102/ | 6,907,112 | LDPred (p = | 0.63 | 0.63 |
| bowel | 21,770 | 120,280 | 288,978 | | 0.1) | (0.62- | (0.62- |
| disease | controls³² | (1.1%) | (1.1%) | | | 0.65) | 0.64) |
| Breast cancer | 122,977 cases/ | 2,576/ | 6,586/ | 5,218 | Pruning and | 0.68 | 0.69 |
| | 105,974 | 63,347 | 157,895 | | thresholding | (0.67- | (0.68- |
| | controls³³ | (4.1%) | (4.2%) | | (r² < 0.2, | 0.69) | 0.69) |
| | | | | | p < 5 × 10⁻⁴) | | |

TABLE 48
Association of candidate polygenic scores with prevalent coronary artery
disease. Odds ratio (OR) per standard deviation (SD) and area under the receiver-operator curve
(AUC) were calculated using logistic regression in a validation dataset of 120,280 participants in
the UK Biobank (adjusted for age, sex, the first four principal components of ancestry and
genotyping array) of which 3,963 had been diagnosed with having coronary artery disease. p—p-
value in discovery GWAS study; r2—linkage disequilibrium pruning threshold; ρ—tuning
parameter to model the proportion of variants assumed to be causal; OR per SD—odds ratio per
standard deviation increment; AUC—area under the receiver operator curve.

| | | N Variants Available/ | | OR per SD | |
|---|---|---|---|---|---|
| Derivation Strategy | Tuning Parameter | N Variants in Score (%) | | (95% CI) | AUC |
| Genome-wide Significant | p < 5 × 10⁻⁸ and r² < 0.2 | 74/74 | (100.0%) | 1.39 (1.35-1.44) | 0.791 |
| Pruning & Thresholding | p < 5 × 10⁻⁸ and r² < 0.4 | 100/100 | (100.0%) | 1.39 (1.35-1.44) | 0.791 |
| Pruning & Thresholding | p < 5 × 10⁻⁸ and r² < 0.6 | 137/137 | (100.0%) | 1.39 (1.35-1.44) | 0.790 |
| Pruning & Thresholding | p < 5 × 10⁻⁸ and r² < 0.8 | 204/204 | (100.0%) | 1.37 (1.33-1.42) | 0.789 |
| Pruning & Thresholding | p < 5 × 10⁻⁶ and r² < 0.2 | 192/192 | (100.0%) | 1.46 (1.42-1.51) | 0.794 |
| Pruning & Thresholding | p < 5 × 10⁻⁶ and r² < 0.4 | 257/257 | (100.0%) | 1.47 (1.42-1.52) | 0.794 |
| Pruning & Thresholding | p < 5 × 10⁻⁶ and r² < 0.6 | 345/345 | (100.0%) | 1.45 (1.41-1.50) | 0.793 |
| Pruning & Thresholding | p < 5 × 10⁻⁶ and r² < 0.8 | 505/505 | (100.0%) | 1.43 (1.38-1.48) | 0.792 |
| Pruning & Thresholding | p < 5 × 10⁻⁴ and r² < 0.2 | 1269/1273 | (99.7%) | 1.53 (1.48-1.58) | 0.797 |
| Pruning & Thresholding | p < 5 × 10⁻⁴ and r² < 0.4 | 1590/1594 | (99.7%) | 1.56 (1.51-1.61) | 0.798 |
| Pruning & Thresholding | p < 5 × 10⁻⁴ and r² < 0.6 | 1997/2001 | (99.8%) | 1.55 (1.50-1.60) | 0.797 |
| Pruning & Thresholding | p < 5 × 10⁻⁴ and r² < 0.8 | 2706/2710 | (99.9%) | 1.53 (1.48-1.58) | 0.797 |
| Pruning & Thresholding | p < 5 × 10⁻² and r² < 0.2 | 56941/57276 | (99.4%) | 1.48 (1.44-1.53) | 0.794 |
| Pruning & Thresholding | p < 5 × 10⁻² and r² < 0.4 | 70491/70831 | (99.5%) | 1.54 (1.49-1.60) | 0.797 |
| Pruning & Thresholding | p < 5 × 10⁻² and r² < 0.6 | 84921/85264 | (99.6%) | 1.57 (1.52-1.63) | 0.798 |
| Pruning & Thresholding | p < 5 × 10⁻² and r² < 0.8 | 105595/105942 | (99.7%) | 1.59 (1.54-1.64) | 0.799 |
| Pruning & Thresholding | p < 5 × 10⁻¹ and r² < 0.2 | 413921/417670 | (99.1%) | 1.44 (1.39-1.49) | 0.792 |
| Pruning & Thresholding | p < 5 × 10⁻¹ and r² < 0.4 | 590581/594406 | (99.4%) | 1.48 (1.43-1.53) | 0.794 |
| Pruning & Thresholding | p < 5 × 10⁻¹ and r² < 0.6 | 768415/772288 | (99.5%) | 1.51 (1.46-1.56) | 0.795 |
| Pruning & Thresholding | p < 5 × 10⁻¹ and r² < 0.8 | 996630/1000544 | (99.6%) | 1.53 (1.48-1.58) | 0.796 |
| Pruning & Thresholding | p < 1 and r² < 0.2 | 634268/641894 | (98.8%) | 1.44 (1.39-1.48) | 0.792 |
| Pruning & Thresholding | p < 1 and r² < 0.4 | 973234/981023 | (99.2%) | 1.48 (1.43-1.52) | 0.794 |
| Pruning & Thresholding | p < 1 and r² < 0.6 | 1349381/1357303 | (99.4%) | 1.50 (1.46-1.55) | 0.795 |
| Pruning & Thresholding | p < 1 and r² < 0.8 | 1848045/1856048 | (99.6%) | 1.52 (1.47-1.57) | 0.796 |
| LDPred Algorithm | ρ = 1 | 6629369/6630150 | (>99.9%) | 1.52 (1.47-1.58) | 0.796 |
| LDPred Algorithm | ρ = 0.3 | 6629369/6630150 | (>99.9%) | 1.53 (1.48-1.58) | 0.796 |
| LDPred Algorithm | ρ = 0.1 | 6629369/6630150 | (>99.9%) | 1.54 (1.49-1.59) | 0.796 |
| LDPred Algorithm | ρ = 0.03 | 6629369/6630150 | (>99.9%) | 1.57 (1.52-1.62) | 0.798 |
| LDPred Algorithm | ρ = 0.01 | 6629369/6630150 | (>99.9%) | 1.62 (1.57-1.68) | 0.801 |
| LDPred Algorithm | ρ = 0.003 | 6629369/6630150 | (>99.9%) | 1.69 (1.63-1.75) | 0.805 |
| LDPred Algorithm | ρ = 0.001 | 6629369/6630150 | (>99.9%) | 1.72 (1.67-1.78) | 0.806 |

TABLE 49
Association of candidate polygenic scores with prevalent atrial fibrillation.
Odds ratio (OR) per standard deviation (SD) and area under the receiver operator curve (AUC)
were calculated using logistic regression in a validation dataset of 120,280 participants in the UK
Biobank (adjusted for age, sex, the first four principal components of ancestry and genotyping
array) of which 2,024 had been diagnosed with atrial fibrillation. p—p-value in discovery
GWAS study; r2—linkage disequilibrium pruning threshold; ρ—tuning parameter to model the
proportion of variants assumed to be causal; OR per SD—odds ratio per standard deviation
increment; AUC—area under the receiver-operator curve.

| | | N Variants Available/ | | OR per SD | |
|---|---|---|---|---|---|
| Derivation Strategy | Tuning Parameter | N Variants in Score (%) | | (95% CI) | AUC |
| Genome-wide Significant | p < 5 × 10⁻⁸ and | 55/55 | (100.0%) | 1.48 (1.43-1.54) | 0.766 |
| | r² < 0.2 | | | | |
| Pruning & Thresholding | p < 5 × 10⁻⁸ and | 78/78 | (100.0%) | 1.52 (1.46-1.58) | 0.768 |
| | r² < 0.4 | | | | |
| Pruning & Thresholding | p < 5 × 10⁻⁸ and | 106/106 | (100.0%) | 1.53 (1.47-1.60) | 0.768 |
| | r² < 0.6 | | | | |
| Pruning & Thresholding | p < 5 × 10⁻⁸ and | 149/149 | (100.0%) | 1.55 (1.49-1.62) | 0.768 |
| | r² < 0.8 | | | | |
| Pruning & Thresholding | p < 5 × 10⁻⁶ and | 161/161 | (100.0%) | 1.51 (1.45-1.58) | 0.767 |
| | r² < 0.2 | | | | |
| Pruning & Thresholding | p < 5 × 10⁻⁶ and | 218/218 | (100.0%) | 1.56 (1.50-1.62) | 0.769 |
| | r² < 0.4 | | | | |
| Pruning & Thresholding | p < 5 × 10⁻⁶ and | 288/288 | (100.0%) | 1.58 (1.51-1.64) | 0.770 |
| | r² < 0.6 | | | | |
| Pruning & Thresholding | p < 5 × 10⁻⁶ and | 383/383 | (100.0%) | 1.60 (1.53-1.67) | 0.770 |
| | r² < 0.8 | | | | |
| Pruning & Thresholding | p < 5 × 10⁻⁴ and | 2304/2327 | (99.0%) | 1.35 (1.29-1.41) | 0.754 |
| | r² < 0.2 | | | | |
| Pruning & Thresholding | p < 5 × 10⁻⁴ and | 2558/2580 | (99.1%) | 1.45 (1.38-1.51) | 0.759 |
| | r² < 0.4 | | | | |
| Pruning & Thresholding | p < 5 × 10⁻⁴ and | 2919/2941 | (99.3%) | 1.51 (1.44-1.58) | 0.763 |
| | r² < 0.6 | | | | |
| Pruning & Thresholding | p < 5 × 10⁻⁴ and | 3445/3474 | (99.2%) | 1.54 (1.47-1.61) | 0.765 |
| | r² < 0.8 | | | | |
| Pruning & Thresholding | p < 5 × 10⁻² and | 122196/123113 | (99.3%) | 1.20 (1.15-1.26) | 0.748 |
| | r² < 0.2 | | | | |
| Pruning & Thresholding | p < 5 × 10⁻² and | 138395/139383 | (99.3%) | 1.26 (1.20-1.31) | 0.750 |
| | r² < 0.4 | | | | |
| Pruning & Thresholding | p < 5 × 10⁻² and | 156473/157515 | (99.3%) | 1.31 (1.25-1.37) | 0.753 |
| | r² < 0.6 | | | | |
| Pruning & Thresholding | p < 5 × 10⁻² and | 180571/181743 | (99.4%) | 1.33 (1.27-1.39) | 0.754 |
| | r² < 0.8 | | | | |
| Pruning & Thresholding | p < 5 × 10⁻¹ and | 872572/880291 | (99.1%) | 1.18 (1.13-1.23) | 0.747 |
| | r² < 0.2 | | | | |
| Pruning & Thresholding | p < 5 × 10⁻¹ and | 1067307/1075829 | (99.2%) | 1.23 (1.17-1.28) | 0.749 |
| | r² < 0.4 | | | | |
| Pruning & Thresholding | p < 5 × 10⁻¹ and | 1272661/1282064 | (99.3%) | 1.26 (1.21-1.32) | 0.750 |
| | r² < 0.6 | | | | |
| Pruning & Thresholding | p < 5 × 10⁻¹ and | 1522420/1532899 | (99.3%) | 1.28 (1.22-1.33) | 0.751 |
| | r² < 0.8 | | | | |
| Pruning & Thresholding | p < 1 and r² < 0.2 | 1491900/1506103 | (99.1%) | 1.17 (1.12-1.23) | 0.747 |
| Pruning & Thresholding | p < 1 and r² < 0.4 | 1842010/1857685 | (99.2%) | 1.22 (1.17-1.28) | 0.749 |
| Pruning & Thresholding | p < 1 and r² < 0.6 | 2246065/2263436 | (99.2%) | 1.26 (1.20-1.32) | 0.750 |
| Pruning & Thresholding | p < 1 and r² < 0.8 | 2765175/2784693 | (99.3%) | 1.27 (1.22-1.33) | 0.751 |
| LDPred Algorithm | ρ = 1 | 6705798/6730541 | (99.6%) | 1.33 (1.27-1.39) | 0.754 |
| LDPred Algorithm | ρ = 0.3 | 6705798/6730541 | (99.6%) | 1.34 (1.28-1.40) | 0.755 |
| LDPred Algorithm | ρ = 0.1 | 6705798/6730541 | (99.6%) | 1.39 (1.32-1.45) | 0.757 |
| LDPred Algorithm | ρ = 0.03 | 6705798/6730541 | (99.6%) | 1.45 (1.39-1.51) | 0.761 |
| LDPred Algorithm | ρ = 0.01 | 6705798/6730541 | (99.6%) | 1.53 (1.47-1.60) | 0.767 |
| LDPred Algorithm | ρ = 0.003 | 6705798/6730541 | (99.6%) | 1.63 (1.56-1.70) | 0.773 |
| LDPred Algorithm* | ρ = 0.001 | 6705798/6730541 | (99.6%) | 1.04 (0.99-1.08) | 0.743 |

TABLE 50
Association of candidate polygenic scores with prevalent type 2 diabetes.
Odds ratio (OR) per standard deviation (SD) and area under the receiver-operator curve (AUC)
were calculated using logistic regression in a validation dataset of 120,280 participants in the UK
Biobank (adjusted for age, sex, the first four principal components of ancestry and genotyping
array) of which 2,785 had been diagnosed with type 2 diabetes. p—p-value in discovery GWAS
study; r2—linkage disequilibrium pruning threshold; ρ—tuning parameter to model the
proportion of variants assumed to be causal. OR per SD—odds ratio per standard deviation
increment; AUC—area under the receiver-operator curve.

| | | N Variants Available/ | | OR per SD | |
|---|---|---|---|---|---|
| Derivation Strategy | Tuning Parameter | N Variants in Score (%) | | (95% CI) | AUC |
| Genome-wide Significant | p < 5 × 10⁻⁸ and r² < 0.2 | 72/72 | (100.0%) | 1.34 (1.30-1.39) | 0.700 |
| Pruning & Thresholding | p < 5 × 10⁻⁸ and r² < 0.4 | 98/98 | (100.0%) | 1.33 (1.28-1.38) | 0.698 |
| Pruning & Thresholding | p < 5 × 10⁻⁸ and r² < 0.6 | 133/133 | (100.0%) | 1.31 (1.26-1.36) | 0.697 |
| Pruning & Thresholding | p < 5 × 10⁻⁸ and r² < 0.8 | 201/201 | (100.0%) | 1.29 (1.25-1.34) | 0.695 |
| Pruning & Thresholding | p < 5 × 10⁻⁶ and r² < 0.2 | 209/209 | (100.0%) | 1.40 (1.35-1.46) | 0.704 |
| Pruning & Thresholding | p < 5 × 10⁻⁶ and r² < 0.4 | 274/274 | (100.0%) | 1.40 (1.34-1.45) | 0.703 |
| Pruning & Thresholding | p < 5 × 10⁻⁶ and r² < 0.6 | 388/388 | (100.0%) | 1.37 (1.32-1.42) | 0.701 |
| Pruning & Thresholding | p < 5 × 10⁻⁶ and r² < 0.8 | 550/551 | (99.8%) | 1.36 (1.31-1.41) | 0.700 |
| Pruning & Thresholding | p < 5 × 10⁻⁴ and r² < 0.2 | 2838/2913 | (97.4%) | 1.36 (1.31-1.41) | 0.701 |
| Pruning & Thresholding | p < 5 × 10⁻⁴ and r² < 0.4 | 3269/3346 | (97.7%) | 1.40 (1.34-1.45) | 0.704 |
| Pruning & Thresholding | p < 5 × 10⁻⁴ and r² < 0.6 | 3858/3937 | (98.0%) | 1.43 (1.37-1.48) | 0.706 |
| Pruning & Thresholding | p < 5 × 10⁻⁴ and r² < 0.8 | 4832/4912 | (98.4%) | 1.43 (1.37-1.48) | 0.705 |
| Pruning & Thresholding | p < 5 × 10⁻² and r² < 0.2 | 145622/151854 | (95.9%) | 1.37 (1.32-1.42) | 0.701 |
| Pruning & Thresholding | p < 5 × 10⁻² and r² < 0.4 | 169289/175728 | (96.3%) | 1.43 (1.38-1.49) | 0.705 |
| Pruning & Thresholding | p < 5 × 10⁻² and r² < 0.6 | 193703/200323 | (96.7%) | 1.48 (1.42-1.53) | 0.708 |
| Pruning & Thresholding | p < 5 × 10⁻² and r² < 0.8 | 226545/233313 | (97.1%) | 1.47 (1.41-1.53) | 0.707 |
| Pruning & Thresholding | p < 5 × 10⁻¹ and r² < 0.2 | 1049001/1107833 | (94.7%) | 1.32 (1.27-1.37) | 0.697 |
| Pruning & Thresholding | p < 5 × 10⁻¹ and r² < 0.4 | 1353005/1414886 | (95.6%) | 1.38 (1.33-1.44) | 0.701 |
| Pruning & Thresholding | p < 5 × 10⁻¹ and r² < 0.6 | 1634296/1698631 | (96.2%) | 1.42 (1.37-1.48) | 0.704 |
| Pruning & Thresholding | p < 5 × 10⁻¹ and r² < 0.8 | 1959214/2025081 | (96.7%) | 1.45 (1.39-1.50) | 0.705 |
| Pruning & Thresholding | p < 1 and r² < 0.2 | 1682488/1794860 | (93.7%) | 1.31 (1.26-1.36) | 0.696 |
| Pruning & Thresholding | p < 1 and r² < 0.4 | 2280565/2399906 | (95.0%) | 1.37 (1.32-1.42) | 0.700 |
| Pruning & Thresholding | p < 1 and r² < 0.6 | 2881225/3006278 | (95.8%) | 1.42 (1.36-1.47) | 0.703 |
| Pruning & Thresholding | p < 1 and r² < 0.8 | 3575137/3703499 | (96.5%) | 1.44 (1.39-1.50) | 0.706 |
| LDPred Algorithm | ρ = 1 | 6893037/6917436 | (99.6%) | 1.52 (1.47-1.58) | 0.714 |
| LDPred Algorithm | ρ = 0.3 | 6893037/6917436 | (99.6%) | 1.53 (1.47-1.59) | 0.714 |
| LDPred Algorithm | ρ = 0.1 | 6893037/6917436 | (99.6%) | 1.55 (1.49-1.61) | 0.716 |
| LDPred Algorithm | ρ = 0.03 | 6893037/6917436 | (99.6%) | 1.59 (1.53-1.65) | 0.720 |
| LDPred Algorithm | ρ = 0.01 | 6893037/6917436 | (99.6%) | 1.65 (1.59-1.71) | 0.725 |
| LDPred Algorithm | ρ = 0.003 | 6893037/6917436 | (99.6%) | 1.15 (1.11-1.20) | 0.687 |
| LDPred Algorithm* | ρ = 0.001 | 6893037/6917436 | (99.6%) | 1.05 (1.02-1.10) | 0.683 |

TABLE 51
Association of candidate polygenic scores with prevalent inflammatory
bowel disease. Odds ratio (OR) per standard deviation (SD) and area under the receiver-operator
curve (AUC) were calculated using logistic regression in a validation dataset of 120,280
participants in the UK Biobank (adjusted for age, sex, the first four principal components of
ancestry and genotyping array) of which 1,360 had been diagnosed with inflammatory bowel
disease. p—p-value in discovery GWAS study; r2—linkage disequilibrium pruning threshold; ρ—
tuning parameter to model the proportion of variants assumed to be causal; OR per SD—odds
ratio per standard deviation increment; AUC—area under the receiver-operator curve.

| | | N Variants Available/ | | OR per SD | |
|---|---|---|---|---|---|
| Derivation Strategy | Tuning Parameter | N Variants in Score (%) | | (95% CI) | AUC |
| Genome-wide Significant | p < 5 × 10⁻⁸ and r² < 0.2 | 288/292 | (98.6%) | 1.40 (1.34-1.47) | 0.614 |
| Pruning & Thresholding | p < 5 × 10⁻⁸ and r² < 0.4 | 475/484 | (98.1%) | 1.31 (1.24-1.38) | 0.582 |
| Pruning & Thresholding | p < 5 × 10⁻⁸ and r² < 0.6 | 800/812 | (98.5%) | 1.23 (1.17-1.30) | 0.567 |
| Pruning & Thresholding | p < 5 × 10⁻⁸ and r² < 0.8 | 1529/1545 | (99.0%) | 1.18 (1.11-1.24) | 0.557 |
| Pruning & Thresholding | p < 5 × 10⁻⁶ and r² < 0.2 | 520/533 | (97.6%) | 1.43 (1.37-1.50) | 0.625 |
| Pruning & Thresholding | p < 5 × 10⁻⁶ and r² < 0.4 | 857/875 | (97.9%) | 1.36 (1.29-1.43) | 0.591 |
| Pruning & Thresholding | p < 5 × 10⁻⁶ and r² < 0.6 | 1334/1356 | (98.4%) | 1.26 (1.19-1.33) | 0.572 |
| Pruning & Thresholding | p < 5 × 10⁻⁶ and r² < 0.8 | 2391/2418 | (98.9%) | 1.19 (1.13-1.26) | 0.560 |
| Pruning & Thresholding | p < 5 × 10⁻⁴ and r² < 0.2 | 2979/3028 | (98.4%) | 1.54 (1.46-1.62) | 0.631 |
| Pruning & Thresholding | p < 5 × 10⁻⁴ and r² < 0.4 | 3817/3875 | (98.5%) | 1.45 (1.38-1.53) | 0.610 |
| Pruning & Thresholding | p < 5 × 10⁻⁴ and r² < 0.6 | 4949/5013 | (98.7%) | 1.34 (1.27-1.42) | 0.587 |
| Pruning & Thresholding | p < 5 × 10⁻⁴ and r² < 0.8 | 7111/7185 | (99.0%) | 1.24 (1.17-1.30) | 0.569 |
| Pruning & Thresholding | p < 5 × 10⁻² and r² < 0.2 | 118775/121914 | (97.4%) | 1.53 (1.44-1.61) | 0.616 |
| Pruning & Thresholding | p < 5 × 10⁻² and r² < 0.4 | 140825/144087 | (97.7%) | 1.58 (1.50-1.67) | 0.629 |
| Pruning & Thresholding | p < 5 × 10⁻² and r² < 0.6 | 163967/167349 | (98.0%) | 1.54 (1.46-1.63) | 0.623 |
| Pruning & Thresholding | p < 5 × 10⁻² and r² < 0.8 | 195815/199334 | (98.2%) | 1.39 (1.31-1.46 | 0.597 |
| Pruning & Thresholding | p < 5 × 10⁻¹ and r² < 0.2 | 812741/842603 | (96.5%) | 1.46 (1.37-1.55) | 0.598 |
| Pruning & Thresholding | p < 5 × 10⁻¹ and r² < 0.4 | 1066545/1098071 | (97.1%) | 1.50 (1.42-1.59) | 0.608 |
| Pruning & Thresholding | p < 5 × 10⁻¹ and r² < 0.6 | 1308728/1341631 | (97.5%) | 1.53 (1.44-1.61) | 0.616 |
| Pruning & Thresholding | p < 5 × 10⁻¹ and r² < 0.8 | 1602425/1636580 | (97.9%) | 1.46 (1.39-1.55) | 0.610 |
| Pruning & Thresholding | p < 1 and r² < 0.2 | 1291770/1349599 | (95.7%) | 1.45 (1.36-1.54) | 0.597 |
| Pruning & Thresholding | p < 1 and r² < 0.4 | 1783031/1844513 | (96.7%) | 1.49 (1.41-1.58) | 0.607 |
| Pruning & Thresholding | p < 1 and r² < 0.6 | 2291513/2356075 | (97.3%) | 1.52 (1.44-1.61) | 0.615 |
| Pruning & Thresholding | p < 1 and r² < 0.8 | 2917090/2984351 | (97.7%) | 1.47 (1.39-1.55) | 0.610 |
| LDPred Algorithm | ρ = 1 | 6882324/6907112 | (99.6%) | 1.58 (1.49-1.66) | 0.628 |
| LDPred Algorithm | ρ = 0.3 | 6882324/6907112 | (99.6%) | 1.58 (1.50-1.67) | 0.629 |
| LDPred Algorithm | ρ = 0.1 | 6882324/6907112 | (99.6%) | 1.61 (1.52-1.70) | 0.633 |
| LDPred Algorithm | ρ = 0.03 | 6882324/6907112 | (99.6%) | 1.55 (1.47-1.64) | 0.625 |
| LDPred Algorithm | ρ = 0.01 | 6882324/6907112 | (99.6%) | 1.28 (1.22-1.35) | 0.580 |
| LDPred Algorithm | ρ = 0.003 | 6882324/6907112 | (99.6%) | 1.21 (1.15-1.27) | 0.563 |
| LDPred Algorithm* | ρ = 0.001 | 6882324/6907112 | (99.6%) | 1.16 (1.10-1.23) | 0.556 |

TABLE 52
Association of candidate polygenic scores with prevalent breast cancer.
Odds ratio (OR) per standard deviation (SD) and area under the curve (AUC) were calculated
using logistic regression in a validation dataset of 63,347 female participants in the UK Biobank
(adjusted for age, the first four principal components of ancestry and genotyping array) of which
2,576 had been diagnosed with having breast cancer. p—p-value in discovery GWAS study; r2—
linkage disequilibrium pruning threshold; ρ—tuning parameter to model the proportion of
variants assumed to be causal; OR per SD—odds ratio per standard deviation increment; AUC—
area under the receiver-operator curve.

| | | N Variants Available/ | | OR per SD | |
|---|---|---|---|---|---|
| Derivation Strategy | Tuning Parameter | N Variants in Score (%) | | (95% CI) | AUC |
| Genome-wide Significant | p < 5 × 10⁻⁸ and r² < 0.2 | 572/577 | (99.1%) | 1.47 (1.42-1.53) | 0.677 |
| Pruning & Thresholding | p < 5 × 10⁻⁸ and r² < 0.4 | 878/884 | (99.3%) | 1.44 (1.39-1.50) | 0.673 |
| Pruning & Thresholding | p < 5 × 10⁻⁸ and r² < 0.6 | 1284/1292 | (99.4%) | 1.39 (1.34-1.45) | 0.666 |
| Pruning & Thresholding | p < 5 × 10⁻⁸ and r² < 0.8 | 1959/1971 | (99.4%) | 1.39 (1.33-1.45) | 0.666 |
| Pruning & Thresholding | p < 5 × 10⁻⁶ and r² < 0.2 | 1151/1165 | (98.8%) | 1.51 (1.45-1.57) | 0.681 |
| Pruning & Thresholding | p < 5 × 10⁻⁶ and r² < 0.4 | 1692/1712 | (98.8%) | 1.48 (1.42-1.54) | 0.677 |
| Pruning & Thresholding | p < 5 × 10⁻⁶ and r² < 0.6 | 2382/2411 | (98.8%) | 1.43 (1.38-1.49) | 0.671 |
| Pruning & Thresholding | p < 5 × 10⁻⁶ and r² < 0.8 | 3588/3624 | (99.0%) | 1.43 (1.37-1.49) | 0.671 |
| Pruning & Thresholding | p < 5 × 10⁻⁴ and r² < 0.2 | 5158/5218 | (98.9%) | 1.56 (1.49-1.62) | 0.685 |
| Pruning & Thresholding | p < 5 × 10⁻⁴ and r² < 0.4 | 6868/6942 | (98.9%) | 1.55 (1.49-1.61) | 0.684 |
| Pruning & Thresholding | p < 5 × 10⁻⁴ and r² < 0.6 | 8945/9036 | (99.0%) | 1.51 (1.45-1.57) | 0.679 |
| Pruning & Thresholding | p < 5 × 10⁻⁴ and r² < 0.8 | 12352/12461 | (99.1%) | 1.50 (1.44-1.56) | 0.678 |
| Pruning & Thresholding | p < 5 × 10⁻² and r² < 0.2 | 114421/115503 | (99.1%) | 1.45 (1.39-1.50) | 0.672 |
| Pruning & Thresholding | p < 5 × 10⁻² and r² < 0.4 | 143235/144508 | (99.1%) | 1.49 (1.43-1.55) | 0.677 |
| Pruning & Thresholding | p < 5 × 10⁻² and r² < 0.6 | 173750/175238 | (99.2%) | 1.50 (1.44-1.56) | 0.678 |
| Pruning & Thresholding | p < 5 × 10⁻² and r² < 0.8 | 217554/219334 | (99.2%) | 1.51 (1.45-1.57) | 0.678 |
| Pruning & Thresholding | p < 5 × 10⁻¹ and r² < 0.2 | 657758/663879 | (99.1%) | 1.38 (1.33-1.44) | 0.665 |
| Pruning & Thresholding | p < 5 × 10⁻¹ and r² < 0.4 | 910344/918115 | (99.2%) | 1.41 (1.36-1.47) | 0.668 |
| Pruning & Thresholding | p < 5 × 10⁻¹ and r² < 0.6 | 1157487/1166909 | (99.2%) | 1.43 (1.38-1.49) | 0.670 |
| Pruning & Thresholding | p < 5 × 10⁻¹ and r² < 0.8 | 1471670/1483324 | (99.2%) | 1.45 (1.39-1.51) | 0.671 |
| Pruning & Thresholding | p < 1 and r² < 0.2 | 997491/1007125 | (99.0%) | 1.38 (1.32-1.43) | 0.664 |
| Pruning & Thresholding | p < 1 and r² < 0.4 | 1469656/1482406 | (99.1%) | 1.41 (1.35-1.47) | 0.668 |
| Pruning & Thresholding | p < 1 and r² < 0.6 | 1968975/1984988 | (99.2%) | 1.43 (1.37-1.49) | 0.669 |
| Pruning & Thresholding | p < 1 and r² < 0.8 | 2612769/2633156 | (99.2%) | 1.44 (1.38-1.50) | 0.670 |
| LDPred Algorithm | ρ = 1 | 7227160/7261712 | (99.5%) | 1.47 (1.41-1.53) | 0.674 |
| LDPred Algorithm | ρ = 0.3 | 7227160/7261712 | (99.5%) | 1.51 (1.45-1.57) | 0.678 |
| LDPred Algorithm | ρ = 0.1 | 7227160/7261712 | (99.5%) | 1.52 (1.46-1.59) | 0.679 |
| LDPred Algorithm | ρ = 0.03 | 7227160/7261712 | (99.5%) | 1.30 (1.25-1.35) | 0.657 |
| LDPred Algorithm* | ρ = 0.01 | 7227160/7261712 | (99.5%) | 1.18 (1.14-1.23) | 0.646 |
| LDPred Algorithm* | ρ = 0.003 | 7227160/7261712 | (99.5%) | 1.12 (1.08-1.17) | 0.642 |
| LDPred Algorithm* | ρ = 0.001 | 7227160/7261712 | (99.5%) | 1.13 (1.08-1.17) | 0.642 |

TABLE 53
Genome-wide polygenic score characteristics for five diseases across
derivation strategies. For each disease, characteristics of genome-wide polygenic scores (GPSs)
are displayed according to derivation strategy of GWAS significant variants only (pruning and
thresholding with p < 5 × 10-8 and r2 < 0.2), the best of the remaining 23 pruning and
thresholding GPSs, and the best of 7 LDPred GPSs. The score with the highest area under the
receiver-operator curve (denoted by bolded font) was carried forward to the testing dataset.

| | | N variants | | |
|---|---|---|---|---|
| | | available/ | | |
| | Derivation | N variants in | Tuning | AUC |
| Disease | strategy | score (%) | parameters | (95% CI) |
| Coronary artery disease | GWAS significant | 74/74 | p < 5 × 10⁻⁸, | 0.791 |
| | variants | (100%) | r² < 0.2 | (0.785- |
| | | | | 0.798) |
| Coronary artery disease | Pruning and | 105,942/105,595 | p < 0.05, | 0.799 |
| | thresholding | (99.67%) | r² < 0.8 | (0.793- |
| | | | | 0.806) |
| Coronary artery disease | LDPred | 6,629,369/ | p = 0.001 | 0.806 |
| | | 6,630,150 | | (0.800- |
| | | (99.99%) | | 0.813) |
| Atrial fibrillation | GWAS significant | 55/55 | p < 5 × 10⁻⁸, | 0.766 |
| | variants | (100%) | r² < 0.2 | (0.757- |
| | | | | 0.776) |
| Atrial fibrillation | Pruning and | 383/383 | p < 5 × 10⁻⁶, | 0.770 |
| | thresholding | (100%) | r² < 0.8 | (0.760- |
| | | | | 0.780) |
| Atrial fibrillation | LDPred | 6,705,798/ | p = 0.003 | 0.773 |
| | | 6,730,541 | | (0.763- |
| | | (99.63%) | | 0.782) |
| Type 2 diabetes | GWAS significant | 72/72 | p < 5 × 10⁻⁸, | 0.700 |
| | variants | (100%) | r² < 0.2 | (0.690- |
| | | | | 0.709) |
| Type 2 diabetes | Pruning and | 193,703/200,323 | p < 0.05, | 0.708 |
| | thresholding | (96.7%) | r² < 0.6 | (0.699- |
| | | | | 0.717) |
| Type 2 diabetes | LDPred | 6,893,037/ | p = 001 | 0.725 |
| | | 6,917,436 | | (0.716- |
| | | (99.65%) | | 0.734) |
| Inflammatory bowel | GWAS significant | 288/292 | p < 5 × 10⁻⁸, | 0.614 |
| disease | variants | (98.6%) | r² < 0.2 | (0.600- |
| | | | | 0.629) |
| Inflammatory bowel | Pruning and | 2979/3028 | p < 5 × 10⁻⁴, | 0.631 |
| disease | thresholding | (98.4%) | r² < 0.2 | (0.619- |
| | | | | 0.645) |
| Inflammatory bowel | LDPred | 6,882,324/ | p = 01 | 0.633 |
| disease | | 6,907,112 | | (0.619- |
| | | (99.64%) | | 0.648) |
| Breast cancer | GWAS significant | 572/577 | p < 5 × 10⁻⁸, | 0.677 |
| | variants | (99.1%) | r² < 0.2 | (0.667- |
| | | | | 0.687) |
| Breast cancer | Pruning and | 5158/5218 | p < 5 × [symbol] < | 0.685 |
| | thresholding | (98.85%) | 0.2 | (0.675- |
| | | | | 0.695) |
| Breast cancer | LDPred | 7,227,160/ | p = 0.1 | 0.679 |
| | | 7,261,712 | | (0.669- |
| | | (99.5%) | | 0.689) |

Applicant used an initial validation dataset of the 120,280 participants in the UK Biobank Phase 1 genotype data release to select the GPS with the best performance, defined as the maximum area under the receiver-operator curve (AUC). Applicant then assessed the performance in an independent testing set comprised of the 288,978 participants in the UK Biobank Phase 2 genotype data release. For each disease, the discriminative capacity within the testing dataset was nearly identical to that observed in the validation dataset.

Taking CAD as an example, our polygenic predictors were derived from a GWAS involving 184,305 participants¹⁶ and evaluated based on their ability to detect the participants in the UK Biobank validation dataset diagnosed with CAD (Table 47). The predictors had AUC ranging from 0.79-0.81 in the validation set, with the best predictor (GPS_{CAD}) involving 6,630,150 variants (Table 48). This predictor performed equivalently well in the testing dataset, with AUC of 0.81.

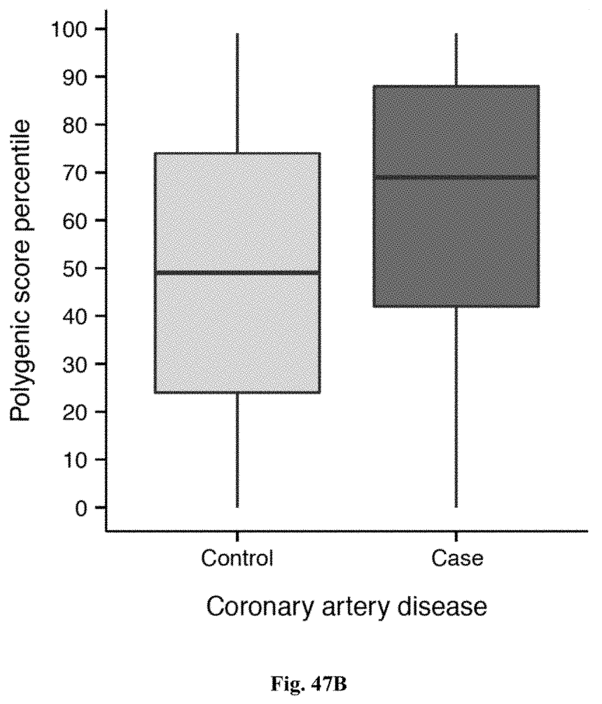

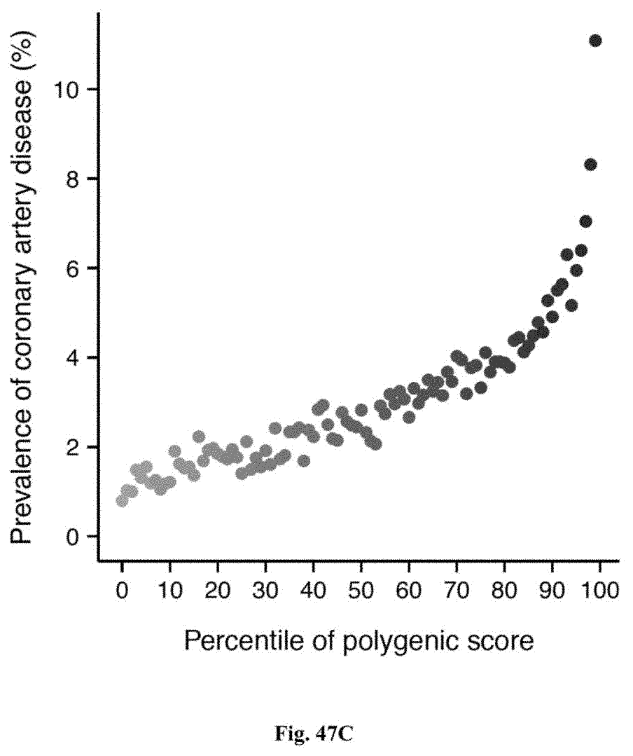

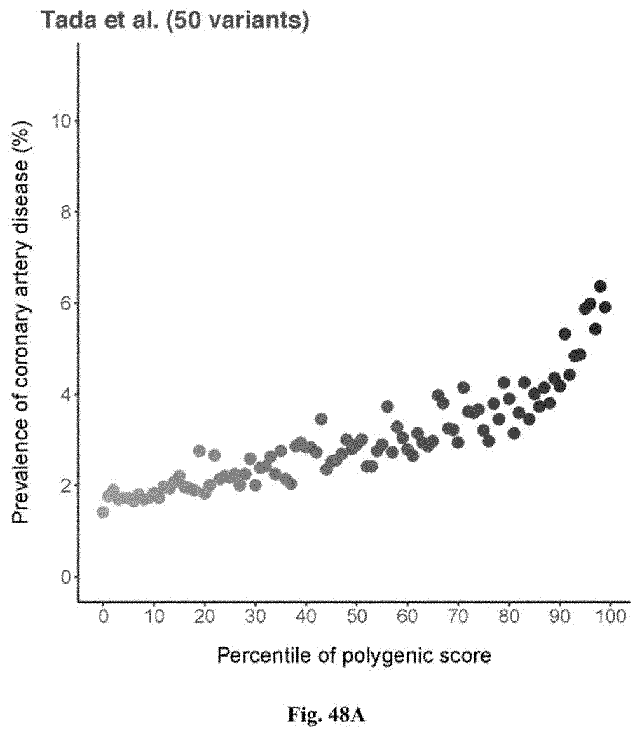

Applicant then investigated whether our polygenic predictor, GPS_{CAD}, could identify individuals at similar risk to the 3-fold increased risk conferred by a familial hypercholesterolemia mutation. Across the population, GPS_{CAD} is normally distributed with the empirical risk of CAD rising sharply in the right tail of the distribution, from 0.8% in the lowest percentile to 11.1% in the highest percentile (FIG. 47). The median GPS_{CAD} percentile score was 69 for individuals with CAD vs. 49 for individuals without CAD. By analogy to the traditional analytic strategy for monogenic mutations, Applicant defined ‘carriers’ as individuals with GPS_{CAD} above a given threshold and ‘non-carriers’ as all others.

Applicant found that 8% of the population had inherited a genetic predisposition that conferred ≥3-fold increased risk for CAD (Table 54).

TABLE 54
Proportion of population at 3, 4, and 5-fold increased risk for each of five
common diseases. For each disease, progressively more extreme tails of the GPS distribution
were compared to the remainder of the population in a logistic regression model with disease
status as the outcome and age, sex, the first four principal components of ancestry, and
genotyping array as predictors. Breast cancer analysis was restricted to female participants.

| High GPS definition | N individuals in population | % of population |
|---|---|---|
| Odds ratio ≥ 3.0 | | |
| Coronary artery disease | 23,119/288,978 | 8.0% |
| Atrial fibrillation | 17,627/288,978 | 6.1% |
| Type 2 diabetes | 10,099/288,978 | 3.5% |
| Inflammatory bowel disease | 9209/288,978 | 3.2% |
| Breast cancer | 2,369/157,895 | 1.5% |
| Any of five diseases | 57,115/288,978 | 19.8% |
| Odds ratio ≥ 4.0 | | |
| Coronary artery disease | 6631/288,978 | 2.3% |
| Atrial fibrillation | 4335/288,978 | 1.5% |
| Type 2 diabetes | 578/288,978 | 0.2% |
| Inflammatory bowel disease | 2297/288,978 | 0.8% |
| Breast cancer | 474/157,895 | 0.3% |
| Any of five diseases | 14,029/288,978 | 4.9% |
| Odds ratio ≥ 5.0 | | |
| Coronary artery disease | 1443/288,978 | 0.5% |
| Atrial fibrillation | 2020/288,978 | 0.7% |
| Type 2 diabetes | 144/288,978 | 0.05% |
| Inflammatory bowel disease | 571/288,978 | 0.2% |
| Breast cancer | 158/157,895 | 0.1% |
| Any of five diseases | 4305/288,978 | 1.5% |

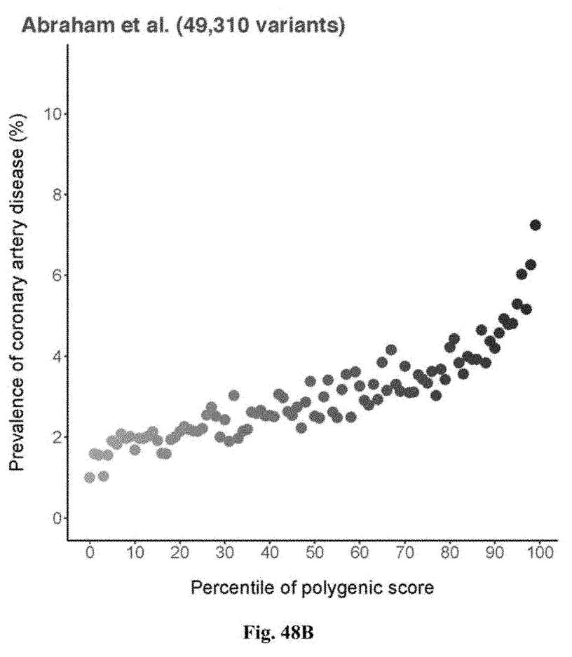

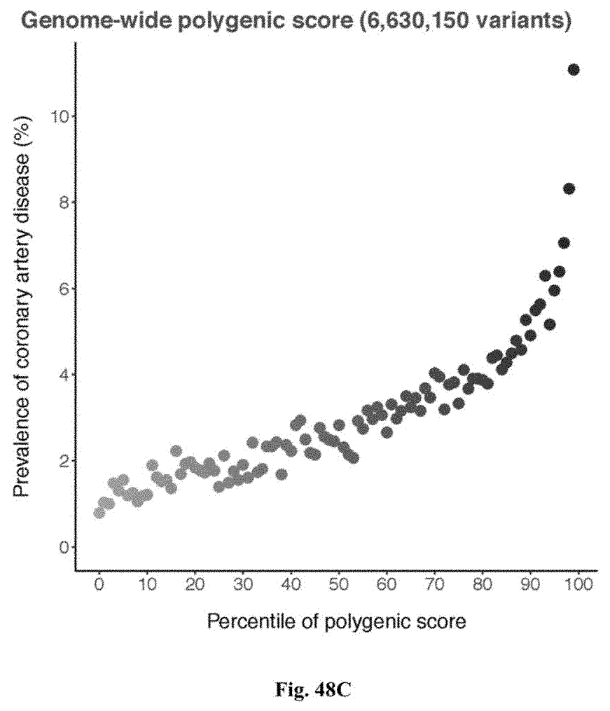

Strikingly, the polygenic score identified 20-fold more people than found by familial hypercholesterolemia mutations in previous studies,^{6,7} at comparable or greater risk. Moreover, 2.3% of the population (‘carriers’) inherited ≥4-fold increased risk for CAD and 0.5% (‘carriers’) had inherited ≥5-fold increased risk. GPS_{CAD} performed substantially better than two previously published polygenic scores for coronary artery disease that included 50 and 49,310 variants, respectively (Table 55 and FIG. 48).

TABLE 55
Comparison of GPSCAD to two previously published polygenic scores for
coronary artery disease. 50 of 50 (100%) of the variants included in the Tada et al. score were
available in the UK Biobank validation dataset. 49,297 of 49,310 (99.97%) of the variants
included in the Abraham et al. score were available in the UK Biobank validation dataset.
6,630,100/6,630,150 (>99.9%) of the variants included in the GPS were available in the UK
Biobank validation dataset. Odds ratios calculated by comparing those with high GPS to the
remainder of the population in a logistic regression model adjusted for age, sex, genotyping
array, and the first four principal components of ancestry.

| | | | | 95% Confidence | |
|---|---|---|---|---|---|
| High GPS definition | Reference group | | Odds ratio | interval | P-value |
| Tada et al.¹ (50 variants) | | | | | |
| Top 20% of distribution | Remaining | 80% | 1.86 | 1.78-1.95 | 2.1 × 10⁻¹⁴³ |
| Top 10% of distribution | Remaining | 90% | 2.09 | 1.97-2.22 | 4.5 × 10⁻¹³⁶ |
| Top 5% of distribution | Remaining | 95% | 2.26 | 2.09-2.43 | 8.6 × 10⁻¹⁰⁰ |
| Top 1% of distribution | Remaining | 99% | 2.24 | 1.90-2.62 | 1.7 × 10⁻²² |
| Top 0.5% of distribution | Remaining | 99.5% | 2.31 | 1.83-2.88 | 3.7 × 10⁻¹³ |
| Abraham et al.² (49,310 variants) | | | | | |
| Top 20% of distribution | Remaining | 80% | 1.94 | 1.85-2.03 | 3.2 × 10⁻¹⁶³ |
| Top 10% of distribution | Remaining | 90% | 2.07 | 1.95-2.19 | 4.5 × 10⁻¹³² |
| Top 5% of distribution | Remaining | 95% | 2.28 | 2.12-2.46 | 1.8 × 10⁻¹⁰³ |
| Top 1% of distribution | Remaining | 99% | 2.71 | 2.33-3.14 | 2.1 × 10⁻³⁹ |
| Top 0.5% of distribution | Remaining | 99.5% | 2.55 | 2.04-3.14 | 1.7 × 10⁻¹⁷ |
| GPS (6,630,150 variants) | | | | | |
| Top 20% of distribution | Remaining | 80% | 2.55 | 2.43-2.67 | <1 × 10⁻³⁰⁰ |
| Top 10% of distribution | Remaining | 90% | 2.89 | 2.74-3.05 | <1 × 10⁻³⁰⁰ |
| Top 5% of distribution | Remaining | 95% | 3.34 | 3.12-3.58 | 6.5 × 10⁻²⁶⁴ |
| Top 1% of distribution | Remaining | 99% | 4.83 | 4.25-5.46 | 1.0 × 10⁻¹³² |
| Top 0.5% of distribution | Remaining | 99.5% | 5.17 | 4.34-6.12 | 7.9 × 10⁻⁷⁸ |

GPS_{CAD} has the advantage that it can be assessed from the time of birth, well before the discriminative capacity emerges for risk factors (for example, hypertension or type 2 diabetes) used in clinical practice to predict CAD. Moreover, even for our middle-aged study population, practicing clinicians could not identify the 8% of individuals at ≥3-fold risk based on GPS_{CAD} in the absence of genotype information (Table 56).

TABLE 56
Baseline characteristics according to high genome-wide polygenic score for
coronary artery disease. Baseline characteristics according to high coronary artery disease
polygenic score status, defined as the top 8% of the distribution empirically shown to be at ≥3-
fold risk of CAD. Values displayed are mean (standard deviation) for continuous variables and N
(%) for categorical variables. GPSCAD—genome-wide polygenic score for coronary artery
disease.

| | Remainder of | | Top 8% of GPS_{CAD} | | |
|---|---|---|---|---|---|
| | population | | distribution | | P-value |
| Number of individuals | 265,859 | | 23,119 | | |
| Coronary artery disease | 7,061 | (2.7%) | 1,615 | (7.0%) | <0.001 |
| Age, years | 56.9 | (8.0) | 56.7 | (8.1) | <0.001 |
| Male sex | 120,673 | (45%) | 10,410 | (45%) | 0.29 |
| Hypertension | 73,982 | (28%) | 7,477 | (32%) | <0.001 |
| Type 2 diabetes | 5,240 | (2.0%) | 613 | (2.7%) | <0.001 |
| Hypercholesterolemia | 35,042 | (13%) | 4,559 | (20%) | <0.001 |
| Current smoking | 24,399 | (9.2%) | 2,200 | (9.5%) | 0.09 |
| Family history of heart disease | 94,117 | (35%) | 10,101 | (44%) | <0.001 |
| Body mass index, kg/m² | 27.3 | (4.7) | 27.6 | (4.8) | <0.001 |
| Lipid-lowering therapy | 43,923 | (17%) | 5,589 | (24%) | <0.001 |

For example, conventional risk factors such as hypercholesterolemia was present in 20% of those with ≥3-fold risk based on GPS_{CAD} versus 13% of those in the remainder of the distribution, hypertension in 32% versus 28%, and family history of heart disease in 44% versus 35%. Making high GPS_{CAD} individuals aware of their inherited susceptibility may facilitate intensive prevention efforts. For example, Applicant previously showed that a high polygenic risk for CAD may be offset by either of two interventions: adherence to a healthy lifestyle or cholesterol-lowering therapy with statin medications.

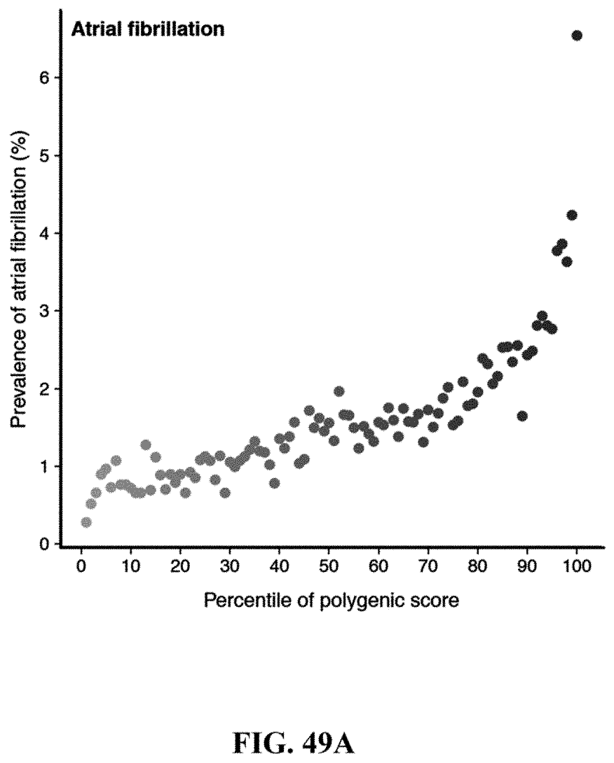

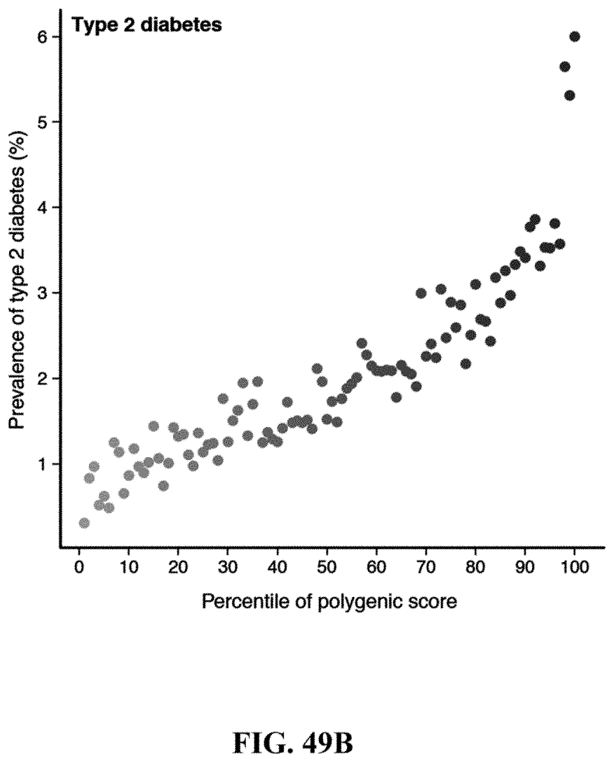

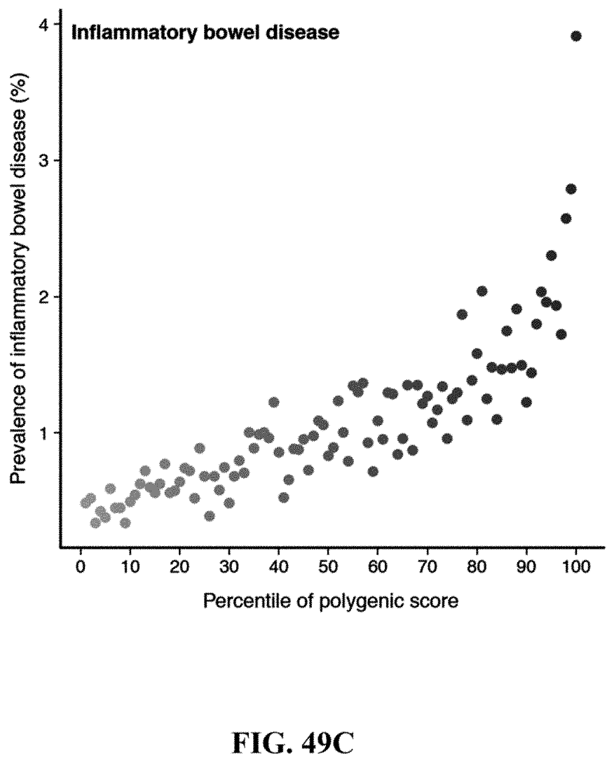

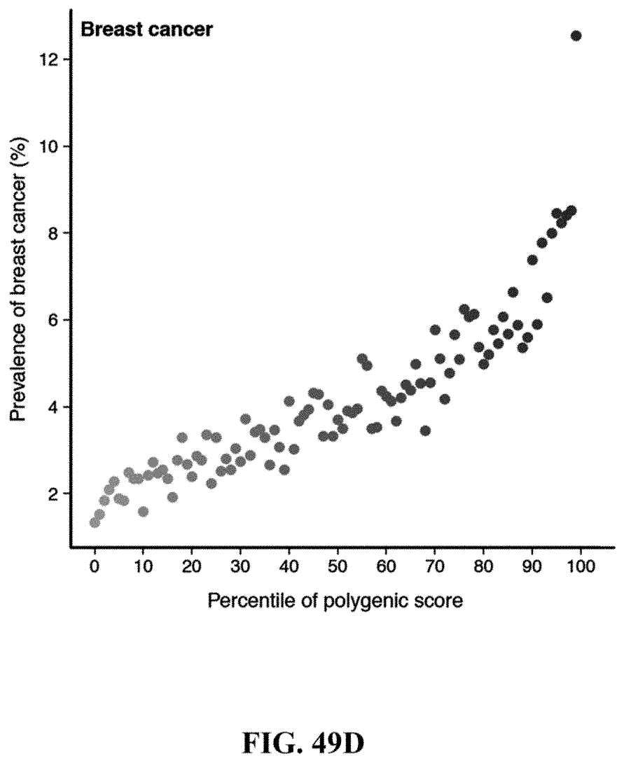

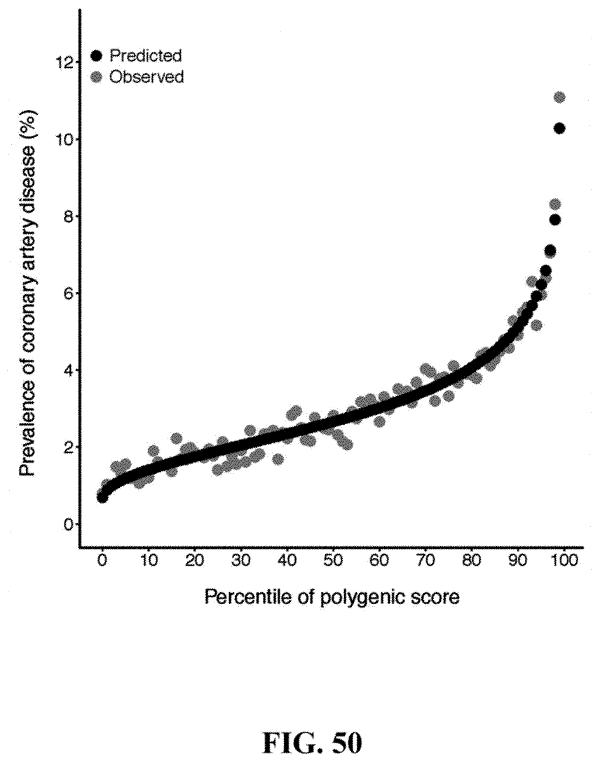

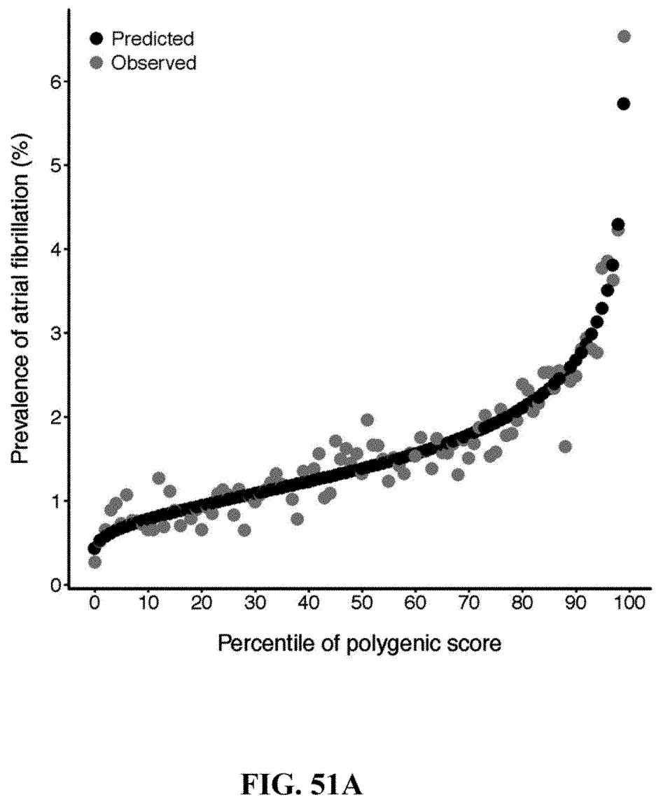

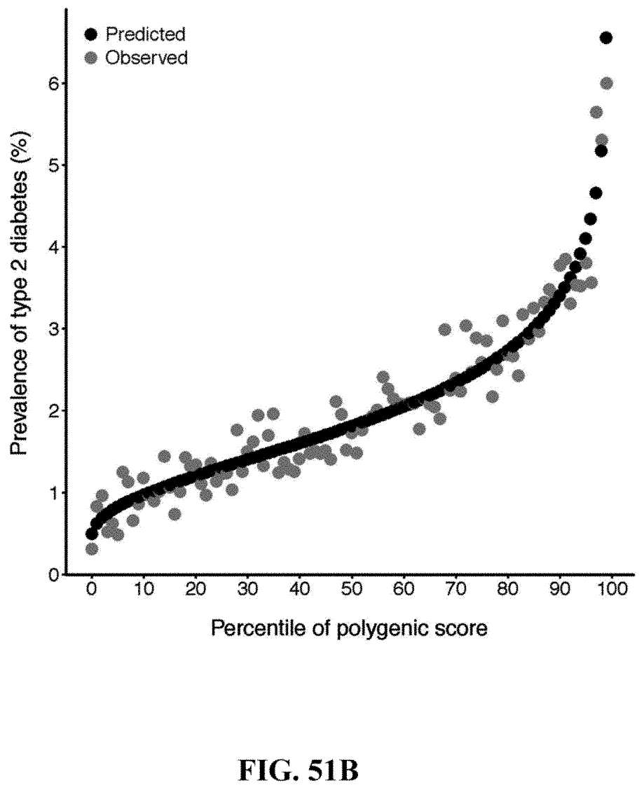

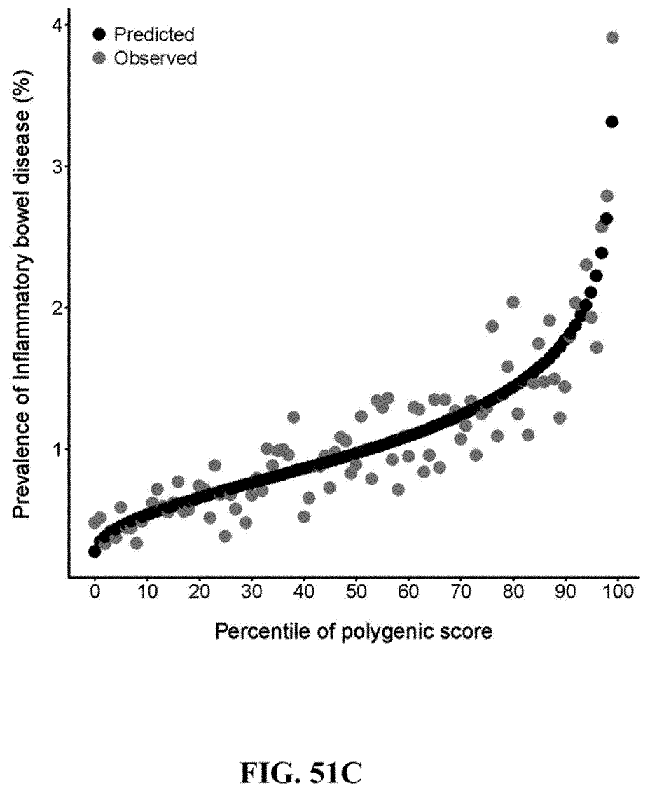

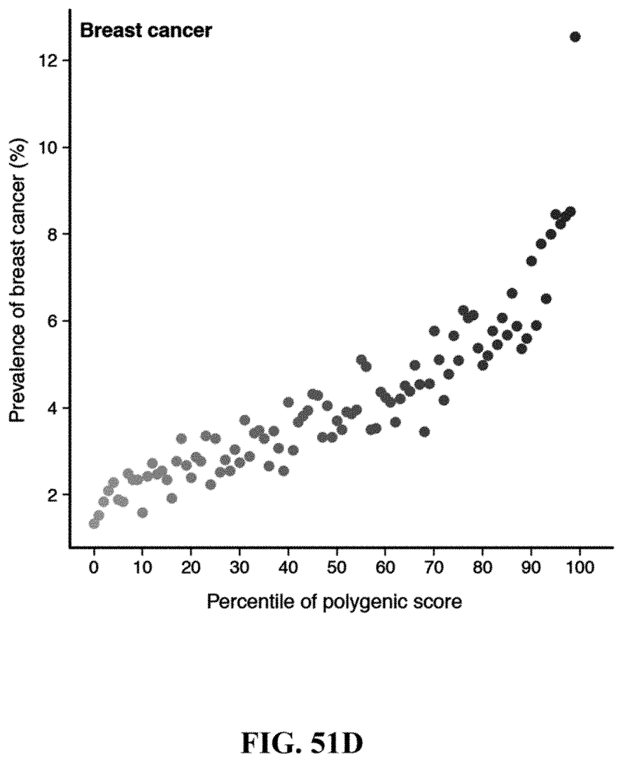

Our results for CAD generalized to four other diseases: risk increased sharply in the right tail of the GPS distribution (FIG. 49). For each disease, the shape of the observed risk gradient was consistent with predicted risk based only on the GPS (FIGS. 50-51).

Atrial fibrillation is an underdiagnosed and often asymptomatic disorder in which an irregular heart rhythm predisposes to blood clots and is a leading cause of ischemic stroke.²² The polygenic predictor identified 6.1% of the population at ≥3-fold risk and the top 1% had 4.63-fold risk (Tables 37 and 40). Screening for atrial fibrillation has become increasingly feasible owing to the development of ‘wearable’ device technology; these efforts to increase detection may have maximal utility in those with high GPS_{AF}.

TABLE 57
Prevalence and clinical impact of a high genome-wide polygenic score.
GPS—genome-wide polygenic score. Odds ratios calculated by comparing those with high GPS
to the remainder of the population in a logistic regression model adjusted for age, sex,
genotyping array, and the first four principal components of ancestry. Breast cancer analysis was
restricted to female participants.

| | | | 95% Confidence | |
|---|---|---|---|---|
| High GPS definition | Reference group | Odds ratio | interval | P-value |
| Coronary artery | | | | |
| disease | | | | |
| Top 20% of | Remaining 80% | 2.55 | 2.43-2.67 | <1 × 10⁻³⁰⁰ |
| distribution | | | | |
| Top 10% of | Remaining 90% | 2.89 | 2.74-3.05 | <1 × 10⁻³⁰⁰ |
| distribution | | | | |
| Top 5% of distribution | Remaining 95% | 3.34 | 3.12-3.58 | 6.5 × 10⁻²⁶⁴ |
| Top 1% of distribution | Remaining 99% | 4.83 | 4.25-5.46 | 1.0 × 10⁻¹³² |
| Top 0.5% of | Remaining 99.5% | 5.17 | 4.34-6.12 | 7.9 × 10⁻⁷⁸ |
| distribution | | | | |
| Atrial fibrillation | | | | |
| Top 20% of | Remaining 80% | 2.43 | 2.29-2.59 | 2.1 × 10⁻¹⁷⁷ |
| distribution | | | | |
| Top 10% of | Remaining 90% | 2.74 | 2.55-2.94 | 7.0 × 10⁻¹⁶⁹ |
| distribution | | | | |
| Top 5% of distribution | Remaining 95% | 3.22 | 2.9-3.51 | 1.1 × 10⁻¹⁵² |
| Top 1% of distribution | Remaining 99% | 4.63 | 3.96-5.39 | 2.9 × 10⁻⁸⁴ |
| Top 0.5% of | Remaining 99.5% | 5.23 | 4.24-6.39 | 3.5 × 10⁻⁵⁶ |
| distribution | | | | |
| Type 2 diabetes | | | | |
| Top 20% of | Remaining 80% | 2.33 | 2.20-2.46 | 3.1 × 10⁻²⁰¹ |
| distribution | | | | |
| Top 10% of | Remaining 90% | 2.49 | 2.34-2.66 | 1.2 × 10⁻¹⁶⁷ |
| distribution | | | | |
| Top 5% of distribution | Remaining 95% | 2.75 | 2.53-2.98 | 1.7 × 10⁻¹³⁰ |
| Top 1% of distribution | Remaining 99% | 3.30 | 2.81-3.85 | 1.4 × 10⁻⁴⁹ |
| Top 0.5% of | Remaining 99.5% | 3.48 | 2.79-4.29 | 4.3 × 10⁻³⁰ |
| distribution | | | | |
| Inflammatory bowel | | | | |
| disease | | | | |
| Top 20% of | Remaining 80% | 2.19 | 2.03-2.36 | 7.7 × 10⁻⁹⁵ |
| distribution | | | | |
| Top 10% of | Remaining 90% | 2.43 | 2.22-2.65 | 8.8 × 10⁻⁸⁸ |
| distribution | | | | |
| Top 5% of distribution | Remaining 95% | 2.66 | 2.38-2.96 | 3.0 × 10⁻⁶⁸ |
| Top 1% of distribution | Remaining 99% | 3.87 | 3.18-4.66 | 1.4 × 10⁻⁴³ |
| Top 0.5% of | Remaining 99.5% | 4.81 | 3.74-6.08 | 9.0 × 10⁻³⁷ |
| distribution | | | | |
| Breast cancer | | | | |
| Top 20% of | Remaining 80% | 2.07 | 1.97-2.19 | 3.4 × 10⁻¹⁵⁹ |
| distribution | | | | |
| Top 10% of | Remaining 90% | 2.32 | 2.18-2.48 | 2.3 × 10⁻¹⁴⁸ |
| distribution | | | | |
| Top 5% of distribution | Remaining 95% | 2.55 | 2.35-2.76 | 2.1 × 10⁻¹¹² |
| Top 1% of distribution | Remaining 99% | 3.36 | 2.88-3.91 | 1.3 × 10⁻⁵⁴ |
| Top 0.5% of | Remaining 99.5% | 3.83 | 3.11-4.68 | 8.2 × 10⁻³⁸ |
| distribution | | | | |

Type 2 diabetes is a key driver of cardiovascular and renal disease, with rapidly increasing global prevalence.²³ The polygenic predictor identified 3.5% of the population at ≥3-fold risk and the top 1% had 3.30-fold risk. (Tables 37 and 40). Both medications and an intensive lifestyle intervention have been proven to prevent progression to type 2 diabetes,²⁴ but widespread implementation has been limited by side effects and cost, respectively. Ascertainment of those with high GPS_{T2D} may provide an opportunity to target such interventions with increased precision.

Inflammatory bowel disease involves chronic intestinal inflammation and often requires lifelong anti-inflammatory medications or surgery to remove afflicted segments of the intestines.²⁵ The polygenic predictor identified 3.2% of the population at ≥3-fold risk and the top 1% had 3.87-fold risk (Tables 37 and 40). Although no therapies to prevent inflammatory bowel disease are currently available, ascertainment of those with increased GPS_{IBD} may enable enrichment of a clinical trial population to assess a novel preventive therapy.

Breast cancer is the leading cause of malignancy-related death in women. The polygenic predictor identified 1.5% of the population at ≥3-fold risk (Tables 37 and 40). Moreover, 0.1% of women had ≥5-fold risk of breast cancer-corresponding to a breast cancer prevalence of 19.0% in this group versus 4.2% in the remaining 99.9% of the distribution. The role of screening mammograms for asymptomatic middle-aged women has remained controversial owing to a low-incidence of breast cancer in this age group and a high false positive rate. Knowledge of GPS_{BC} may inform clinical decision making about the appropriate age to recommend screening.

The results above show that, for a number of common diseases, polygenic risk scores can now identify a substantially larger fraction of the population than found by rare monogenic mutations, at comparable or greater disease risk. Our validation and testing were performed in the UK Biobank population. Individuals who volunteered for the UK Biobank tended to be more healthy than the general population; although this nonrandom ascertainment is likely to deflate disease prevalence, the relative impact of genetic risk strata can be generalizable across study populations. Additional studies are warranted to develop polygenic risk scores for many other common diseases with large GWAS data and validate risk estimates within population biobanks and clinical health systems.

Polygenic risk scores differ in important ways from the identification of rare monogenic risk factors. Whereas identifying carriers of rare monogenic mutations requires sequencing of specific genes and careful interpretation of the functional effects of mutations found, polygenic scores can be readily calculated for many diseases simultaneously, based on data from a single genotyping array. In our testing dataset, 19.8% of participants were at ≥3-fold increased risk for at least one of the five diseases studied (Table 37).

The potential to identify individuals at significantly higher genetic risk, across a wide range of common diseases and at any age, poses a number of opportunities for clinical medicine. Prevention and detection strategies may have utility regardless of underlying mechanism—as is the case for statin therapy for CAD, blood thinning-medications to prevent stroke in those with atrial fibrillation, or intensified mammography screening for breast cancer.

### Methods

### Polygenic Score Derivation

Polygenic scores provide a quantitative metric of an individuals inherited risk based on the cumulative impact of many common polymorphisms. Weights are generally assigned to each genetic variant according to the strength of their association with disease risk (effect estimate). Individuals are scored based on how many risk alleles they have for each variant (for example, 0, 1, or 2 copies) included in the polygenic score.

For our score derivation, Applicant used summary statistics from recent GWAS studies conducted primarily among participants of European ancestry for five diseases and a linkage disequilibrium reference panel of 503 European samples from 1000 Genomes phase 3 version 5. UK Biobank samples were not included in any of the five discovery GWAS studies. DNA polymorphisms with ambiguous strand (A/T or C/G) were removed from the score derivation. For each disease, Applicant computed a set of candidate genome-wide polygenic scores (GPS) using the LDPred algorithm and a pruning and threshold derivation strategies.

The LDPred computational algorithm was used to generate seven candidate GPSs for each disease. This Bayesian approach calculates a posterior mean effect size for each variant based on a prior and subsequent shrinkage based on the extent to which this variant is correlated with similarly associated variants in the reference population. The underlying Gaussian distribution additionally considers the fraction of causal (e.g. non-zero effect sizes) markers via a tuning parameter, ρ. Because p is unknown for any given disease, a range of ρ, the fraction of causal variants, was used—1, 0.3, 0.1, 0.03, 0.01, 0.003, 0.001.

A second approach, pruning and thresholding, was used to build an additional 24 candidate GPSs. Pruning and thresholding scores were built using a p-value and LD-driven clumping procedure in PLINK version 1.90b (clump). In brief, the algorithm forms clumps around SNPs with association p-values less than a provided threshold. Each clump contains all SNPs within 250 kb of the index SNP that are also in LD with the index SNP as determined by a provided r² threshold in the LD reference. The algorithm iteratively cycles through all index SNPs, beginning with the smallest p-value, only allowing each SNP to appear in one clump. The final output should contain the most significantly disease-associated SNP for each LD-based clump across the genome. A GPS was built containing the index SNPs of each clump with association estimate betas (log odds) as weights. GPSs were created over a range of p-value (1, 0.5, 0.05, 5×10⁻⁴, 5×10⁻⁶, 5×10⁻⁸) and r² (0.2, 0.4, 0.6, 0.8) thresholds, for a total of 24 pruning and thresholding-based candidate scores for each disease. The resulting GPS for a p-value threshold of 5×10⁻⁸ and r² of <0.2 was denoted the ‘GWAS significant variant’ derivation strategy.

### Polygenic Score Calculation in the Validation Dataset

For each disease, the thirty-one candidate GPSs were calculated in a validation dataset of 120,280 participants of European ancestry derived from the UK Biobank Phase I release. The UK Biobank is a large prospective cohort study that enrolled individuals from across the United Kingdom, aged 40-69 years at time of recruitment, starting in 2006.¹⁴ Individuals underwent a series of anthropometric measurements and surveys, including medical history review with a trained nurse.

Scores were generated by multiplying the genotype dosage of each risk allele for each variant by its respective weight, and then summing across all variants in the score using PLINK2 software.³⁵ Incorporating genotype dosages accounts for uncertainty in genotype imputation. The vast majority of variants in the GPSs were available for scoring purposes in the validation dataset with sufficient imputation quality (INFO >0.3) (Tables 31-36).

For each of the five diseases, the score with the best discriminative capacity was determined based on maximal area under the receiver-operator curve (AUC) in a logistic regression model with the disease as the outcome and the disease-specific candidate GPS, age, sex, first four principal components of ancestry, and an indicator variable for genotyping array used (Tables 31-36). AUC confidence intervals were calculated using the “pROC” package within R.

### Testing Cohort

The testing dataset was comprised of 288,978 UK Biobank Phase 2 participants distinct from those in the validation dataset described above. Individuals in the UK Biobank underwent genotyping with one of two closely related custom arrays (UK BiLEVE Axiom Array or UK Biobank Axiom Array) consisting of over 800,000 genetic markers scattered across the genome. Additional genotypes were imputed centrally using the Haplotype Reference Consortium resource, the UK10K panel, and the 1000 Genomes panel. In order to analyze individuals with a relatively homogenous ancestry and owing to small percentages of non-British individuals, the present analysis was restricted to the white British ancestry individuals. This subpopulation was constructed centrally using a combination of self-reported ancestry and genetically confirmed ancestry using principal components. Additional exclusion criteria included outliers for heterozygosity or genotype missing rates, discordant reported versus genotypic sex, putative sex chromosome aneuploidy, or withdrawal of informed consent, derived centrally as previously reported.

For each of the five diseases, proportion of variance explained was calculated for each disease using the Nagelkerke's pseudo-R2 metric (Table 58). The R2 was calculated for the full model inclusive of the genome-wide polygenic score plus the covariates minus R² for the covariates alone, thus yielding an estimate of the explained variance. Covariates in the model included age, gender, genotyping array, and the first four principal components of ancestry.

TABLE 58
Assessment of genome-wide polygenic scores in the testing dataset.
Proportion of variance explained was calculated for each disease using the Nagelkerke's pseudo-
R2 metric. The R2 was calculated for the full model inclusive of the genome-wide polygenic
score plus the covariates minus R2 for the covariates alone, thus yielding an estimate of the
explained variance attributable to the polygenic score. Covariates in the model included age,
gender, genotyping array, and the first four principal components of ancestry.

| | N variants available/ | Proportion of variance |
|---|---|---|
| Disease | variants in score (%) | explained (%) |
| Coronary artery disease | 6,630,100/6,630,150 | 4.0% |
| | (>99.9%) | |
| Atrial fibrillation | 6,722,280/6,730,541 | 2.9% |
| | (99.9%) | |
| Type 2 diabetes | 6,909,367/6,917,436 | 2.9% |
| | (99.9%) | |
| Inflammatory bowel disease | 6,899,007/6,907,112 | 2.1% |
| | (99.9%) | |
| Breast cancer | 5,186/5,218 | 2.7% |
| | (99.4%) | |

A sensitivity analysis was performed by removing one individual from each pair of related individuals (third-degree or closer; kinship coefficient >0.0442), confirming similar results within this subpopulation comprised of 222,529 of the 288,978 (77%) testing dataset participants (Table 59).

TABLE 59
Prevalence and clinical impact of a high genome-wide polygenic score in
unrelated individuals. GPS—genome-wide polygenic score. A sensitivity analysis was
performed in 222,529 of 288,978 (77%) of the validation cohort after excluding one of each pair
of related individuals (third-degree or closer). Odds ratios calculated by comparing those with
high GPS to the remainder of the population in a logistic regression model adjusted for age, sex,
genotyping array, and the first four principal components of ancestry. Breast cancer analysis was
restricted to female participants.

| | | | 95% Confidence | |
|---|---|---|---|---|
| High GPS definition | Reference group | Odds ratio | interval | P-value |
| Coronary artery disease | | | | |
| Top 20% of distribution | Remaining 80% | 2.53 | 2.42-2.66 | <1 × 10⁻³⁰⁰ |
| Top 10% of distribution | Remaining 90% | 2.90 | 2.74-3.07 | <1 × 10⁻³⁰⁰ |
| Top 5% of distribution | Remaining 95% | 3.34 | 3.11-3.58 | 1.6 × 10⁻²⁴⁴ |
| Top 1% of distribution | Remaining 99% | 4.53 | 3.95-5.17 | 5.2 × 10⁻¹⁰⁸ |
| Top 0.5% of distribution | Remaining 99.5% | 5.18 | 4.31-6.20 | 1.6 × 10⁻⁷⁰ |
| Atrial fibrillation | | | | |
| Top 20% of distribution | Remaining 80% | 2.47 | 2.31-2.65 | 6.7 × 10⁻¹⁵⁹ |
| Top 10% of distribution | Remaining 90% | 2.74 | 2.52-2.96 | 7.2 × 10⁻¹³⁶ |
| Top 5% of distribution | Remaining 95% | 3.17 | 2.87-3.49 | 5.4 × 10⁻¹¹⁹ |
| Top 1% of distribution | Remaining 99% | 4.42 | 3.78-5.36 | 1.4 × 10⁻⁶⁴ |
| Top 0.5% of distribution | Remaining 99.5% | 5.27 | 4.15-6.60 | 4.4 × 10⁻⁴⁵ |
| Type 2 diabetes | | | | |
| Top 20% of distribution | Remaining 80% | 2.37 | 2.23-2.52 | 4.2 × 10⁻¹⁶⁸ |
| Top 10% of distribution | Remaining 90% | 2.52 | 2.35-2.71 | 2.3 × 10⁻¹³⁸ |
| Top 5% of distribution | Remaining 95% | 2.77 | 2.53-3.03 | 1.5 × 10⁻¹⁰⁶ |
| Top 1% of distribution | Remaining 99% | 3.36 | 2.81-3.99 | 1.8 × 10⁻⁴¹ |
| Top 0.5% of distribution | Remaining 99.5% | 3.42 | 2.67-4.33 | 2.5 × 10⁻²³ |
| Inflammatory bowel disease | | | | |
| Top 20% of distribution | Remaining 80% | 2.19 | 2.01-2.38 | 9.1 × 10⁻⁷³ |
| Top 10% of distribution | Remaining 90% | 2.51 | 2.27-2.77 | 4.1 × 10⁻⁷⁴ |
| Top 5% of distribution | Remaining 95% | 2.75 | 2.42-3.10 | 1.9 × 10⁻⁵⁷ |
| Top 1% of distribution | Remaining 99% | 3.72 | 2.96-4.62 | 8.4 × 10⁻³¹ |
| Top 0.5% of distribution | Remaining 99.5% | 4.47 | 3.31-5.89 | 1.4 × 10⁻²⁴ |
| Breast cancer | | | | |
| Top 20% of distribution | Remaining 80% | 2.08 | 1.96-2.21 | 3.2 × 10⁻¹²² |
| Top 10% of distribution | Remaining 90% | 2.36 | 2.20-2.54 | 6.8 × 10⁻¹¹⁸ |
| Top 5% of distribution | Remaining 95% | 2.59 | 2.36-2.84 | 1.5 × 10⁻⁸⁹ |
| Top 1% of distribution | Remaining 99% | 3.47 | 2.91-4.12 | 4.4 × 10⁻⁴⁵ |
| Top 0.5% of distribution | Remaining 99.5% | 3.78 | 2.97-4.75 | 9.7 × 10⁻²⁹ |

Diagnosis of prevalent disease was based on a composite of data from self-report in an interview with a trained nurse, electronic health record (EHR) information including inpatient International Classification of Disease (ICD-10) diagnosis codes and Office of Population and Censuses Surveys (OPCS-4) procedure codes.

Coronary artery disease ascertainment was based on a composite of myocardial infarction or coronary revascularization. Myocardial infarction was based on self-report or hospital admission diagnosis, as performed centrally. This included individuals with ICD-9 codes of 410.X, 411.0, 412.X, 429.79 or ICD-10 codes of I21.X, I22.X, I23.X, 124.1, 125.2 in hospitalization records. Coronary revascularization was assessed based on an OPCS-4 coded procedure for coronary artery bypass grafting (K40.1-40.4, K41.1-41.4, K45.1-45.5) or coronary angioplasty with or without stenting (K49.1-49.2, K49.8-49.9, K50.2, K75.1-75.4, K75.8-75.9).

Atrial fibrillation ascertainment was based on self-report of atrial fibrillation, atrial flutter, or cardioversion in an interview with a trained nurse, ICD-9 codes of 427.3 or ICD-10 codes of I48.X in hospitalization records, or history of a percutaneous ablation or cardioversion based on OPCS-4 coded procedure (K57.1, K62.1, K62.2, K62.3, K 62.4) as performed previously.

Type 2 diabetes ascertainment was based on self-report in an interview with a trained nurse or ICD-10 codes of E11.X in hospitalization records. Inflammatory bowel disease ascertainment was based on report in an interview with a trained nurse, ICD-9 codes of 555.X or ICD-10 codes of K51.X in hospitalization records.

Breast cancer ascertainment was based on self-report in an interview with a trained nurse, ICD-9 codes (174, 174.9) or ICD-10 codes (C50.X) in hospitalization records, or a breast cancer diagnosis reported to the national registry prior to date of enrollment.

Statistical Analysis within the Testing Dataset

For each disease, the GPS with the best discriminative capacity in the testing dataset was calculated in the testing dataset of 288,278 participants using genotyped and imputed variants using the Hail software package.³⁶ The proportion of the population and of diseased individuals with a given magnitude of increased risk was determined by comparing progressively more extreme tails of the distribution to the remainder of the population in a logistic regression model predicting disease status and adjusted for age, gender, four principal components of ancestry, and genotyping array. Individuals were next binned into 100 groupings according to percentile of the GPS and unadjusted prevalence of disease within each bin determined. Applicant next compared the observed risk gradient across percentile bins to that which would be predicted by the GPS. For each individual, the predicted probability of disease was calculated using a logistic regression model with only the genome-wide polygenic score (GPS) as a predictor. The predicted prevalence of disease within each percentile bin of the GPS distribution was calculated as the average predicted probability of all individuals within that bin. The shape of the predicted risk gradient was consistent with the empirically observed risk gradient for each of the five diseases (FIGS. 50-51). Statistical analyses were conducted using R version 3.4.3 software (The R Foundation).

### REFERENCES

Green E D, Guyer M S; National Human Genome Research Institute. Charting a course for genomic medicine from base pairs to bedside. Nature. 470, 204-213 (2011).
Fisher, R. A. The correlation between relatives on the supposition of Mendelian inheritance. Proc. Roy. Soc. Edinburgh 52, 99-433 (1918).
Gibson G. Rare and common variants: twenty arguments. Nat Rev Genet. 18, 135-45 (2012).
Golan D, Lander E S, Rosset S. Measuring missing heritability: inferring the contribution of common variants. Proc Natl Acad Sci USA. 111, E5272-81 (2014).
Fuchsberger C, et al. The genetic architecture of type 2 diabetes. Nature. 536, 41-47 (2016).
Abul-Husn N. S., et al. Genetic identification of familial hypercholesterolemia within a single U.S. health care system. Science. 354 (2016).
Nordestgaard, B. G., et al. Familial hypercholesterolaemia is underdiagnosed and undertreated in the general population: guidance for clinicians to prevent coronary heart disease: consensus statement of the European Atherosclerosis Society. Eur Heart J. 34, 3478-90a (2013).
Lek M, et al. Analysis of protein-coding genetic variation in 60,706 humans. Nature. 536, 285-91 (2016).
Estrada K, et al. Association of a low-frequency variant in HNF1A with type 2 diabetes in a Latino population. JAMA. 311, 2305-14 (2014).
Chatterjee, N. et al. Projecting the performance of risk prediction based on polygenic analyses of genome-wide association studies. Nat Genet. 45, 400-405 (2013).
Zhang Y., et al. Estimation of complex effect-size distributions using summary-level statistics from genome-wide association studies across 32 complex traits and implications for the future. Preprint at: www.biorxiv.org/content/early/2017/08/11/175406 (2017).
Ripatti S, et al. A multilocus genetic risk score for coronary heart disease: case-control and prospective cohort analyses. Lancet. 327, 1393-400 (2010).
Vilhjálmsson, B. J. et al. Modeling linkage disequilibrium increases accuracy of polygenic scores. Am J Hum Genet. 97, 576-592 (2015).
Sudlow, C. et al. UK biobank: an open access resource for identifying the causes of a wide range of complex diseases of middle and old age. PLoS Med. 12, e1001779 (2015).
Bycroft C, et al. Genome-wide genetic data on ˜500,000 UK Biobank participants. Preprint at: www.biorxiv.org/content/early/2017/07/20/166298 (2017).
Nikpay, M. et al. A comprehensive 1,000 Genomes-based genome-wide association meta-analysis of coronary artery disease. Nat Genet. 47, 1121-1130 (2015).
Tada H, et al. Risk prediction by genetic risk scores for coronary heart disease is independent of self-reported family history. Eur Heart J. 37, 561-7 (2016).
Abraham G., et al. Genomic prediction of coronary heart disease. Eur Heart J. 37, 3267-3278 (2016).
Khera, A. V., et al. Genetic risk, adherence to a healthy lifestyle, and coronary disease. N Engl J Med. 375, 2349-2358 (2016).
Mega, J. L., et al. Genetic risk, coronary heart disease events, and the clinical benefit of statin therapy: an analysis of primary and secondary prevention trials. Lancet. 385, 2264-2271 (2015).
Natarajan, P., et al. Polygenic risk score identifies subgroup with higher burden of atherosclerosis and greater relative benefit from statin therapy in the primary prevention setting. Circulation. 135, 2091-2101 (2017).
January, C. T., et al. 2014 AHA/ACC/HRS guideline for the management of patients with atrial fibrillation: a report of the American College of Cardiology/American Heart Association Task Force on practice guidelines and the Heart Rhythm Society. Circulation. 130, e199-267 (2014).
GBD 2015 Disease and Injury Incidence and Prevalence Collaborators. Global, regional, and national incidence, prevalence, and years live with disability for 310 diseases and injuries, 1990-2015: a systematic analysis for the Global Burden of Disease Study 2015. Lancet. 388, 1545-1602 (2016).
Knowler W. C., et al. Reduction in the incidence of type 2 diabetes with lifestyle intervention or metformin. N Engl J Med. 346, 393-403 (2002).
Abraham, C. & Cho, J. H. Inflammatory bowel disease. N Engl J Med. 361, 2066-78 (2009).
Pharoah P D, Antoniou A C, Easton D F, Ponder B A. Polygenes, risk prediction, and targeted prevention of breast cancer. N Engl J Med. 358, 2796-803 (2008).
Fry A., et al. Comparison of sociodemographic and health-related characteristics of UK Biobank participants with those of the general population. Am J Epidemiol. 186, 1026-34 (2017).
Khera A. V. & Kathiresan S. Is coronary atherosclerosis one disease or many? Setting realistic expectations for precision medicine. Circulation. 135, 1005-07 (2017).
Martin, A. R. et al. Human demographic history impacts genetic risk prediction across diverse populations. Am J Hum Genet. 100, 635-649 (2017).
Christophersen, I. E., et al. Large-scale analyses of common and rare variants identify 12 new loci associated with atrial fibrillation. Nat Genet. 49, 946-952 (2017).
Scott, R. A., et al. An Expanded Genome-Wide Association Study of Type 2 Diabetes in Europeans. Diabetes. 66, 2888-2902 (2017).
Liu J Z, et al. Association analyses identify 38 susceptibility loci for inflammatory bowel disease and highlight shared genetic risk across populations. Nat Genet. 47, 979-986 (2015).
Michailidou K, et al. Association analysis identifies 65 new breast cancer risk loci. Nature. 551, 92-94 (2017).
The 1000 Genomes Project Consortium. A global reference for human genetic variation. Nature. 526, 68-74 (2015).
Chang C C, et al. Second-generation PLINK: rising to the challenge of larger and richer datasets. GigaScience. 4, 7 (2015).
Ganna A, et al. Ultra-rare disruptive and damaging mutations influence educational attainment in the general population. Nat Neurosci. 19, 1563-65 (2016).

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A method of determining a risk of developing atrial fibrillation in a subject, the method comprising:
identifying whether at least 95 single nucleotide polymorphisms (SNPs) from Table A are present in a biological sample from the subject;
wherein the presence of a risk allele of a SNP from Table A indicates that the subject has an increased risk of atrial fibrillation, and wherein the presence of an alternative allele indicates that the subject has a decreased risk of atrial fibrillation.

2. The method of claim 1, further comprising calculating a polygenic risk score (PRS).

3. The method of claim 2, wherein the PRS is calculated by summing the weighted risk score associated with each SNP identified.

4. The method of claim 1, wherein identifying comprises measuring the presence of the at least 95 SNPs in the biological sample.

5. The method of claim 2, further comprising assigning the subject to a risk group based on the PRS.

6. The method of claim 1, further comprising an initial step of obtaining a biological sample from the subject.

7. The method of claim 1, wherein at least 100 SNPs are identified.

8. The method of claim 1, wherein at least 200 SNPs, or at least 500 SNPs, or at least 1000 SNPs, or at least 2000 SNPs, or at least 5000 SNPs, or at least 10,000 SNPs, or at least 20,000 SNPs, or at least 50,000 SNPs, or at least 75,000 SNPs, or at least 100,000 SNPs, or at least 500,000 SNPs, or at least 1,000,000 SNPs, or at least 2,000,000 SNPs, or at least 3,000,000 SNPs, or at least 4,000,000 SNPs, or at least 5,000,000 SNPs, or at least 6,000,000 SNPs are identified.

9. The method of claim 1, wherein the identified SNPs comprise the highest risk SNPs.

10. The method of claim 1, wherein the identified SNPs comprise one or more of rs10841443, rs2244608, rs7500448, rs2972146, rs2972146, and rs11057401.

11. The method of claim 1, further comprising initiating a treatment to the subject.

12. The method of claim 11, wherein the treatment is determined or adjusted according to the risk of atrial fibrillation.

13. The method of claim 1, wherein the treatment comprises statins, ezetimibe, beta-blocking agents, angiotensin-converting-enzyme inhibitors, aspirin, anticoagulants, antiplatelet agents, angiotensin II receptor blockers, angiotensin receptor neprilysin inhibitors, calcium channel blockers, cholesterol-lowering medications, vasodilators, antidiuretics, renin-angiotensin system agents, lipid-modifying medicines, anti-inflammatory agents, nitrates, antiarrhythmic medicines, steroidal or non-steroidal anti-inflammatory drugs, DNA methyltransferase inhibitors and/or histone deacetylase inhibitors.

14. The method of claim 1, wherein identifying whether the SNP is present comprises sequencing at least part of a genome of one or more cells from the subject.

15. The method of claim 13, wherein the DNA methyltransferase inhibitors comprise 5-aza-2′-deoxycytidine or 5-azacytidine.

16. The method of claim 13, wherein the histone deacetylase inhibitors comprise varinostat, romidepsin, panobinostat, belinostat or entinostat.

17. The method of claim 13, wherein the lipid-modifying medicines comprise an antagonist of PCSK9, an antisense oligonucleotide targeting apolipoprotein C-III, and an antisense oligonucleotide to lower lipoprotein(a).

18. The method of claim 13, wherein the statins comprise atorvastatin, fluvastatin, lovastatin, pravastatin, rosuvastatin, and simvastatin.

19. The method of claim 1, wherein the subject is a human.

20. The method of claim 13, wherein sequencing comprises whole genome sequencing.

21. A method of identifying a risk of developing atrial fibrillation in a subject and providing a treatment to the subject, the method comprising:
obtaining a biological sample from the subject; and
identifying whether at least one single nucleotide polymorphism (SNP) from Table A is present in the biological sample;
wherein the presence of a risk allele of a SNP from Table A indicates that the subject has an increased risk of atrial fibrillation; and
initiating a treatment to the subject, wherein the treatment comprises statins, ezetimibe, beta-blocking agents, angiotensin-converting-enzyme inhibitors, aspirin, anticoagulants, antiplatelet agents, angiotensin II receptor blockers, angiotensin receptor neprilysin inhibitors, calcium channel blockers, cholesterol-lowering medications, vasodilators, antidiuretics, renin-angiotensin system agents, lipid-modifying medicines, anti-inflammatory agents, nitrates, antiarrhythmic medicines, steroidal or non-steroidal anti-inflammatory drugs, DNA methyltransferase inhibitors and/or histone deacetylase inhibitors.

22. A method of detecting single nucleotide polymorphisms in a subject, said method comprising:
detecting whether at least 95 single nucleotide polymorphisms (SNPS) from Table A are present in a biological sample from a subject by contacting the biological sample with a set of probes to each SNP and detecting binding of the probes, by amplifying genome regions comprising the SNPs using a set of amplification primers, or by sequencing genomic regions comprising or enriched for the SNPs.

23. The method of claim 22, wherein at least 100 SNPs are detected.

24. The method of claim 22, wherein at least 200 SNPs, or at least 500 SNPs, or at least 1000 SNPs, or at least 2000 SNPs, or at least 5000 SNPs, or at least 10,000 SNPs, or at least 20,000 SNPs, or at least 50,000 SNPs, or at least 75,000 SNPs, or at least 100,000 SNPs, or at least 500,000 SNPs, or at least 1,000,000 SNPs, or at least 2,000,000 SNPs, or at least 3,000,000 SNPs, or at least 4,000,000 SNPs, or at least 5,000,000 SNPs, or at least 6,000,000 SNPs are detected.

25. A method of determining a polygenic risk score for (PRS) developing atrial fibrillation in a subject, the method comprising:
selecting at least 95 single nucleotide polymorphisms (SNPs) from Table A;
identifying whether the at least 95 SNPs are present in a biological sample from the subject; and calculating the polygenic risk score (PRS) based on the presence of the SNPs.

26. A method of reducing a risk of atrial fibrillation in a subject comprising administering to the subject a treatment which comprises one or more statins, beta-blocking agents, angiotensin-converting-enzyme inhibitors, aspirin, anticoagulants, antiplatelet agents, angiotensin II receptor blockers, angiotensin receptor neprilysin inhibitors, calcium channel blockers, cholesterol-lowering medications, vasodilators, antidiuretics, renin-angiotensin system agents, lipid-modifying medicines, anti-inflammatory agents, nitrates, antiarrhythmic medicines, steroidal or non-steroidal anti-inflammatory drugs, DNA methyltransferase inhibitors and/or histone deacetylase inhibitors,
wherein the subject has a polygenic risk score that corresponds to a high risk group, and
wherein the polygenic risk score is calculated by a method according to claim 25.


SEQUENCE LISTING (2 sequences)

SEQ ID NO: 1 - protein, 254 aa, Artificial Sequence
  Synthetic Peptide
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQ

SEQ ID NO: 2 - protein, 183 aa, Artificial Sequence
  Synthetic Peptide
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS